(19) United States
(12) Patent Application Publication
(10) Pub. No.: US 2025/0360137 A1
(43) Pub. Date: Nov. 27, 2025

(54) ENHANCEMENT OF THE EFFICACY OF SEROTONERGIC PSYCHEDELIC DRUGS IN THE TREATMENT OR PREVENTION OF CERTAIN NEUROPSYCHIATRIC DISORDERS THROUGH INHIBITION OF TYPE-9 PHOSPHODIESTERASES

(71) Applicant: Freedom Biosciences, Inc., Tiburon, CA (US)

(72) Inventors: Christopher John Turner Pittenger, Bethany, CT (US); Frank Samuel Menniti, Mystic, CT (US); Jeffrey Sprouse, New York, NY (US)

(21) Appl. No.: 19/206,221

(22) Filed: May 13, 2025

Related U.S. Application Data
(63) Continuation of application No. PCT/US23/37272, filed on Nov. 14, 2023.
(60) Provisional application No. 63/383,732, filed on Nov. 15, 2022.

(51) Int. Cl. A61K 31/519; A61K 31/4045; A61K 31/4745; A61K 31/675; A61K 45/06; A61P 25/18

(52) U.S. Cl. CPC A61K 31/519; A61K 31/4045; A61K 31/4745; A61K 31/675; A61K 45/06; A61P 25/18

(57) ABSTRACT

The present application provides compositions and methods for treating diseases or disorders using a therapeutically effective amount of a serotonergic psychedelic drug and a therapeutically effective amount of a phosphodiesterase type 9 inhibitor.

### CROSS-REFERENCE

This application is a continuation of International Patent Application No. PCT/US2023/37272, filed Nov. 14, 2023, which claims the benefit of U.S. Provisional Application No. 63/383,732, filed Nov. 15, 2022, each of which is incorporated herein by reference in its entirety.

### BACKGROUND

Major depressive disorder (MDD), also known colloquially as depression, is a mental disorder characterized by at least two weeks of continuously low mood or lack of pleasure, interest, and motivation, often accompanied by low self-esteem, low energy, changes in sleep, appetite, and ability to concentrate, guilty ruminations, preoccupation with death, and unexplained pain. MDD negatively affects sufferers' social lives, performance in work and other roles, and general health, and it leads to suicide in 2-15% of affected adults. Up to 60% of suicide victims have experienced MDD or another mood disorder. Patients with MDD are often treated with counseling, structured psychotherapy, or antidepressants. Unfortunately, the efficacy of currently available treatments is modest, and many patients continue to suffer despite aggressive deployment of available treatments. In addition, many patients do not receive evidence-based treatment, due to limited availability and other failings of our healthcare system or to patient reluctance to seek help.

Numerous other psychiatric conditions are often comorbid with major depressive disorder and/or overlap in symptomatology, and perhaps in pathophysiology. For example, other primary mood disorders include bipolar disorder type 1 and 2, cyclothymic disorder, and persistent depressive disorder (formerly known as dysthymia). Other relevant disorders include obsessive-compulsive disorder (OCD) and OCD-related disorders (hoarding disorder, body dysmorphic disorder, trichotillomania, excoriation, illness anxiety disorder, Tourette syndrome) and post-traumatic stress disorder (PTSD). Certain antidepressants and psychotherapies are used for the treatment of these and related conditions; as in the case of MDD, efficacy is limited for many patients, and new treatments are urgently needed.

Traditional antidepressants (ADs), e.g. SSRI antidepressants such as fluoxetine, are used to treat MDD as well as a range of other psychiatric conditions, including posttraumatic stress disorder (PTSD), obsessive-compulsive disorder (OCD), borderline personality disorder, and several other mood and anxiety disorders. However, these drugs can take weeks to months to exert full therapeutic effects; they are herein described as ‘slowly acting antidepressant drugs’, or SAADs. SAADs require daily treatment and are often accompanied by troubling side effects. Furthermore, up to 50% of treated patients do not achieve clinically significant improvement in their treatments and continue to suffer. There is an urgent need for improved therapies for treatment of these psychiatric conditions.

A new class of rapidly acting antidepressant drugs (RAADs) promises to fundamentally change the treatment of MDD, OCD, PTSD, and related psychiatric conditions. The paradigmatic RAAD, ketamine, was found in the late 1990s to produce antidepressant effects in many patients within an hour, persisting for days out to 1-2 weeks. Ketamine is now used in clinical practice, with a typical protocol entailing twice-weekly infusions for several weeks, followed by a gradual tapering of frequency. Esketamine, the purified S-enantiomer of racemic ketamine, has been approved by the US Food and Drug Administration for the treatment of MDD and of suicidality; like ketamine, it is typically administered repeatedly, but not daily. More recently, serotonergic psychedelic drugs such as psilocybin have been found, in small controlled studies and developing pilot work, to have beneficial effects in MDD, addictive disorders, OCD, and other psychiatric conditions. Similarly, 3,4-methoxy-diaminodextroamphetamine (MDMA) and other drugs of the entactogen/empathogen class have been found to provide benefit in PTSD and other conditions. The psychedelics and the entactogens/empathogens are not FDA-approved, and many of them are regulated as Schedule 1 substances by the U.S. Drug Enforcement Agency; they are not available for widespread clinical use but are being vigorously investigated for a range of indications.

While these RAADs are beginning to change psychiatric practice, they remain limited. The benefits of ketamine and esketamine, while rapid, can be short-lived (days to weeks), making repeated treatment necessary in some instances. Early evidence suggests that the psilocybin and other psychedelics may produce longer-lived benefits, though this is not yet rigorously established; as a result current practice with these drugs (in research settings) entails lengthy, labor-intensive engagement with teams of skilled clinicians, increasing costs and complicating their dissemination (if and when they receive regulatory approval).

RAAD in general, and serotonergic psychedelic drugs in particular, are exciting new additions to the therapeutic arsenal available for the treatment of MDD, other mood disorders, and other neuropsychiatric conditions such as PTSD, OCD, and addictive disorders; ketamine and esketamine have entered into clinical practice, and serotonergic psychedelic drugs are being vigorously researched. Nevertheless, these agents have important limitations. The effects of RAADs can be time-limited, and treatment is costly and burdensome, especially for serotonergic psychedelics, given the duration of acute psychological effects and requirement for labor-intensive monitoring and support, as these agents are currently administered in the research setting. These observations highlight the need for new strategies to extend the duration of benefit after treatment with serotonergic psychedelic drugs, to reduce burdensome side effects (non-limiting examples of which include psychedelic effects, dissociation, and cardiovascular effects), and in other ways to reduce the cost and burden of treatment such that it can be more widely disseminated.

### BRIEF SUMMARY

Provided herein is a method for treating or preventing a disorder in a subject comprising: administering to the subject a therapeutically effective amount of a serotonergic psychedelic drug in conjunction with an inhibitor of one or more brain phosphodiesterase type 9 (PDE9s). In some embodiments, the disorder comprises a neuropsychiatric disorder, cluster headache, or migraine headache. In some embodiments, the PDE9 inhibitor enhances a therapeutic effect of the serotonergic psychedelic drug on the disorder to be treated. In some embodiments, the PDE9 inhibitor prolongs at least one biological activity of the serotonergic psychedelic drug in the subject. In some embodiments, the PDE9 inhibitor mitigates at least one undesirable biological activity of the serotonergic psychedelic drug in the subject. In some embodiments, the PDE9 inhibitor reduces the abuse potential of the serotonergic psychedelic drug in the subject.

In some embodiments, the neuropsychiatric disorder is selected from the group consisting of a major depressive disorder (MDD, including major depressive episode), a major depressive episode in bipolar disorder (bipolar depression), depressive episodes associated with bipolar I or II disorder (bipolar depression), a persistent depressive disorder (dysthymia), a disruptive mood dysregulation disorder, a premenstrual dysphoric disorder, a substance/medication-induced depressive disorder, a depressive disorder due to another medical condition, other specified depressive disorder, unspecified depressive disorder, an anxiety disorder, an obsessive-compulsive disorder, a posttraumatic stress disorder, an addictive disorder, treatment resistant depression (TRD), a generalized anxiety disorder (GAD), a post-traumatic stress disorder (PTSD), adjunctive treatment of major depression, eating disorders including anorexia and bulimia, depression associated with neurodegenerative disorders such as Parkinson's Disease, Alzheimer's Disease, dementias, and ALS, traumatic brain injury, suicidal thoughts and behaviors, chronic pain, a persistent depressive disorder, seasonal affective disorder (SAD), psychotic depression, peripartum (postpartum) depression, a premenstrual dysphoric disorder (PMDD), situational depression, and any combinations thereof.

In some embodiments, the serotonergic psychedelic drug is selected from the group of 1) Classical psychedelics, non-limiting examples of which include psilocybin, dimethyltryptamine (DMT), 5-methoxydimethyltryptamine (5-MeO-DMT), lysergic acid diamide (LSD), and lysergic acid amide (LSA); 2) Entactogens/Empathogens, a non-limiting example of which is 3′4′-methylenedioxy-methamphetamine (MDMA); 3) Other phenethylamines, non-limiting examples of which include mescaline, 2,5-dimethoxy-4-iodoamphetamine (DOI), 2,5-dimethoxy-4-methylamphetamine (DOM), and 2,5-dimethoxy-4-bromophenethylamine (2C-B).

In some embodiments, the serotonergic psychedelic drug is a tryptamine or an ergoline. In some embodiments, the serotonergic psychedelic drug comprises a tryptamine, or a salt, solvate, enantiomer, or diastereomer thereof. In some embodiments, the serotonergic psychedelic drug comprises an ergoline, or a salt, solvate, enantiomer, or diastereomer thereof. In some embodiments, the serotonergic psychedelic drug is psilocybin, or a salt, solvate, enantiomer, or diastereomer thereof.

In some embodiments, the phosphodiesterase inhibitor is selected from the group of: 1) Nonselective phosphodiesterase inhibitors, non-limiting examples of which include aminophylline, pentoxifylline, and theobromine; 2) Phosphodiesterase type 9 inhibitors, non-limiting examples of which include paraxanthine, PF-04447913, PF-04447943, PF-04447953, BAY 73-6691, E 2027, CRD 773, BI 409306, CRD 740, TT 00920, BAY 7081, FRM 16606, HUC1-288, WYQ-C36D, and any combinations thereof. In some embodiments, the PDE9 inhibitor is PF-04447943, or a derivative thereof, or a salt, solvate, enantiomer or diastereomer thereof.

In some embodiments, the psychedelic and the PDE9 inhibitor are co-administered to the subject. In some embodiments, the PDE9 inhibitor is administered to the subject before the psychedelic is administered to the subject. In some embodiments, the psychedelic is administered to the subject before the PDE9 inhibitor is administered to the subject. In some embodiments, the psychedelic and the PDE9 inhibitor are co-formulated as a pharmaceutical composition. In some embodiments, the psychedelic and the PDE9 inhibitor are independently administered to the subject by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, intravenous, and any combinations thereof. In some embodiments, the subject is administered with a therapeutically effective amount of psilocybin and PF-04447943. In some embodiments, the subject is administered with a therapeutically effective amount of psilocybin, or a derivative thereof, or a salt, solvate, enantiomer or diastereomer thereof and PF-04447943, or a derivative thereof, or a salt, solvate, enantiomer or diastereomer thereof.

Provided herein is a pharmaceutical composition comprising a serotonergic psychedelic drug and a PDE9 inhibitor, wherein the serotonergic psychedelic drug and the PDE9 inhibitor are present in amounts whereby administration of the pharmaceutical composition to a subject treats or prevents a neuropsychiatric disorder; and wherein the PDE9 inhibitor mitigates at least one side effect of the psychedelic on the subject. In some embodiments, the pharmaceutical composition is formulated for administration by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous.

In some embodiments, the PDE9 inhibitor enhances a therapeutic effect of the serotonergic psychedelic drug on the disorder to be treated. In some embodiments, the PDE9 inhibitor prolongs at least one biological activity of the serotonergic psychedelic drug in the subject. In some embodiments, the PDE9 inhibitor mitigates at least one undesirable biological activity of the serotonergic psychedelic drug in the subject. In some embodiments, the PDE9 inhibitor reduces the abuse potential of the serotonergic psychedelic drug in the subject.

In some embodiments, the serotonergic psychedelic drug is selected from the group consisting of Psilocin, Psilocybin, Bufotenin, Baeocystin, Aeruginascin, 5-MeO-DMT, N,N-Dimethyltryptamine (DMT), 5-Bromo-DMT, N-Methyl-N-ethyltryptamine (MET), N-Methyl-N-isopropyltryptamine (MiPT), N-Methyl-N-propyltryptamine (MPT), N,N-Diethyltryptamine (DET), N-Ethyl-N-isopropyltryptamine (EiPT), N-Methyl-N-butyltryptamine (MBT), N-Propyl-N-isopropyltryptamine (PiPT), N,N-Dipropyltryptamine (DPT), N,N-Diisopropyltryptamine (DiPT), N,N-Diallyltryptamine (DALT), N,N-Dibutyltryptamine (DBT), N-Ethyltryptamine (NET), N-Methyltryptamine (NMT), Trimethyltryptamine (TMT), α-Methyltryptamine, α-Ethyltryptamine, α,N-DMT, α,N,N-Trimethyltryptamine, Ethocybin, 4-HO-MET, 4-HO-DET, 4-HO-MPT, 4-HO-MiPT, 4-HO-MALT, 4-HO-DPT, 4-HO-DiPT, 4-HO-DALT, 4-HO-DBT, 4-HO-DSBT, 4-HO-uMT, 4-HO-MPMI, 4-HO-TMT, 4-HO-1,N,N-TMT, 4-HO-5-MeO-DMT, 4-AcO-DMT, 4-AcO-MET, 4-AcO-MALT, 4-AcO-DET, 4-AcO-EiPT, 4-AcO-DPT, 4-AcO-DiPT, 4-AcO-DALT, 4-MeO-DMT, 4-MeO-MiPT, 5-MeO-N MT, 5-MeO-MET, 5-MeO-MPT, 5-MeO-MiPT, 5-MeO-DET, 5-MeO-Ei PT, 5-MeO-EPT, 5-MeO-Pi PT, 5-MeO-DPT, 5-MeO-DiPT, 5-MeO-DALT, 5-MeO-uMT, 5-MeO-2,N,N-TMT, 5-MeO-7,N,N-TMT, 5-MeO-α,N-DMT, 4-F-5-MeO-DMT, 5-Me-MiPT, 5-HO-DiPT, 2-α-DMT, 4-Me-uMT, 4-Me-αET, 7-Me-αET, 4,5-DHP-AMT, 4,5-DH P-DMT, 4,5-MDO-DMT, 4,5-MDO-DiPT, 5,6-MDO-DiPT, 5,6-MDO-MiPT, 5-Fluoro-uMT, 6-Fluoro-uMT, 6-Fluoro-DMT, N,N-Tetramethyl enetryptamine (Pyr-T), 4-HO-pyr-T, 5-MeO-pyr-T, RU-28306 (4,a-Methylene-N,N-DMT), O-4310 (6-Fluoro-1-Isopropyl-4-HO-DMT), CP-132,484 (4,5-DHP-1-Methyltryptamine), Dimemebfe (5-MeO-BFE), 5-MeO-DiBF, Ibogaine, Voacangine, Lysergic acid diethylamide (LSD), Lysergic acid amide (LSA), Lysergic acid diamide, N1-Methyl-lysergic acid diethylamide, 1-Propionyl-lysergic acid diethylamide, 1-cyclopropanoyl-d-lysergic acid diethylamide, 1-valeryl-D-lysergic acid diethylamide, 6-Allyl-6-nor-lysergic acid diethylamide, 6-Butyl-6-nor-lysergic acid diethylamide, 6-Ethyl-6-nor-lysergic acid diethylamide, 1-Propionyl-6-Ethyl-6-nor-lysergic acid diethylamide, 6-Propyl-6-nor-lysergic acid diethylamide, 6-Cyclopropyl-6-nor-lysergic acid diethylamide, 6-nor-Lysergic acid diethylamide, Lysergic acid ethylamide, Lysergic acid α-hydroxyethylamide, Lysergic acid 2-butyl amide, Lysergic acid 3-pentyl amide, Lysergic acid methyl ester, Lysergic acid 2,4-dimethylazetidide, Lysergic acid piperidine, N,N-Dimethyl-lysergamide, Methylisopropyllysergamide, N,N-Diallyllysergamide, N-Pyrrolidyllysergamide, N-Morpholinyllysergamide, 1-methyl-lysergic acid butanolamide, Lysergic acid 0-propanolamide, Lysergic acid 1-butanolamide, Mescaline, Lophophine, Isomescaline, Cyclopropylmescaline, Thioisomescaline (2-TIM, 3-TIM, and 4-TIM), 4-Desoxymescaline, Jimscaline, Escaline, Metaescaline, Thiometaescaline (3-TME, 4-TME, and 5-TME), Trisescaline, Thiotrisescaline (3-T-TRIS and 4-T-TRIS), Symbescaline, Asymbescaline, Thiosymbescaline (3-TSB and 4-TSB), Phenescaline, Allylescaline, Methallylescaline, Proscaline, Isoproscaline, Metaproscaline, Thioproscaline, Buscaline, Thiobuscaline, α-ethylmescaline, Ariadne, Macromerine, MEPEA, TOM (2-TOM and 5-TOM), Bis-TOM, TOMSO (2-methoxy-4-methyl-5-methylsulfinylamphetamine), TOET (2-TOET and 5-TOET), BOH, BOM (13-Methoxy-mescaline), beta-D, 4-D, DME, F-2, F-22, FLEA, MDPH, MDMP, Propynyl, 2C family (2,5-dimethoxy, 4-substituted phenethylamines), βk-2C-B, 2C-B, 2CB-2EtO, 2CB-5EtO, 2CB-diEtO, 2C-B-FLY, 2C-B-BUTTERFLY, 2C—C, 2C-D, 2CD-2EtO, 2CD-diEtO, 2CD-5EtO, 2C-E, 2C-EF, 2C-F, 2C-G (2C-G-1, 2C-G-2, 2C-G-3, 2C-G-4, 2C-G-5, 2C-G-6, and 2C-G-N), 2C—H, 2C—I, 2CI-2EtO, 2C-iP, 2C-N, 2C-O, 2C-O-4, 2C-P, 2C-SE, 2C-T, 2CT-5EtO, 2C-T-2, 2CT-2-2EtO, 2CT-2-5EtO, 2CT-2-diEtO, 2C-T-4 (2C-T-4 and Ψ-2C-T-4), 2CT-4-2EtO, 2C-T-7, 2CT-7-2EtO, 2C-T-8, 2C-T-9, 2C-T-13, 2C-T-15, 2C-T-16, 2C-T-17, 2C-T-19, 2C-T-21, 2C-TFM, 2C-YN, BOB (I3-Methoxy-2C-B), BOD (I3-Methoxy-2C-D), BOHD (I3-Hydroxy-2C-D), HOT-2, HOT-7, HOT-17, Indane derivatives comprising 2CB-Ind, Benzocyclobutene derivatives comprising 2C-BCB (TCB-2), NBOMe derivatives comprising NBOMe-mescaline, 2C-H—NBOMe, 2C—C—NBOMe, 2CBCB—NBOMe, 2CBFly-NBOMe, 2C—B—NBOMe, 2C—I—NBOMe, 2C-TFM-NBOMe, 2C-D-NBOMe, 2C-G-NBOMe, 2C-E-NBOMe, 2C—P—NBOMe, 2C-iP-NBOMe, 2C—CN—NBOMe, 2C—N—NBOMe, 2C-T-NBOMe, 2C-T-4-NBOMe, 2C-T-7-NBOMe, and DMBMPP, NBOH derivatives comprising 2C—C—NBOH, 2C—B—NBOH, 2C—I—NBOH, and 2C—CN—NBOH, NBMD derivatives comprising 2C—I-NBMD, NBF derivatives comprising 2C—C—NBF, 2C—B—NBF, and 2C—I—NBF, 3C family (3,5-dimethoxy, 4-substituted amphetamines) comprising 3C-E, 3C—P, 3C-DFE, and 3C—BZ, DOx family (2,5-dimethoxy, 4-substituted amphetamines) comprising DOAM, DOB, Meta-DOB, Methyl-DOB, DOBU, DOC, DOEF, DOET, DOI, DOM, Ψ-DOM, DON, DOPR, SOiPR, DOT, Meta-DOT, Ortho-DOT, and DOTFM, DMCPA, DMMDA, DMMDA-2, 2,5-dimethoxy-3,4-dimethylamphetamine, 4-methyl-2,5-dimethoxymethamphetamine, 2,N-dimethyl-4,5-methylenedioxyamphetamine, Dimethoxy amphetamine (2,4-DMA, 2,5-DMA, and 3,4-DMA), Trimethoxyamphetamine (TMA-2, TMA-6), Tetramethoxyamphetamine, Br-DragonFLY, TFMFly, 2-Bromo-4,5-methylenedioxyamphetamine, 4-Bromo-3,5-dimethoxyamphetamine, EEE, EEM, EME, EMM, EDMA, EIDA, Ethyl-J, Methyl-J, Ethyl-K, Mehtyl-K, IDNNA, Iris, MDAI, MDMAI, MDAT, MDMAT, MDAL, MDBU, MDBZ, MDDM, MDIP, MDMEOET, MDMEO, MDOH, MDHOET, MDPL, MDCPK, MDPR, MEDA, MEM, Mehtyl-DMA, MMDA, MMDA-2, 5-Mehtyl-MDA, MEE, MME, MPM, DiFMDA, 5-APB, 6-APB, 5-APDB, 6-APDB, 5-MAPB, 5-MAPDB, 6-MAPDB, 6-MAPB, 6-EAPB, 5-EAPB, Para-Methoxyamphetamine, Paramethoxymethamphetamine, 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Benzoxazine, Efavirenz, Substituted methylenedioxy-phenethylamines (MDxx) comprising 3,4-methylenedioxymethamphetamine (MDMA), MDA, 2,3-MDA, 5-Methyl-MDA, MMDA, MDEA, MBDB, MDAL, MDBU, MDBZ, MDDM, MDIP, MDMEOET, MDMEO, MDOH, MDHOET, MDPL, MDCPM, MDPR, BDB, MMDA-2, DiFMDA, EIDA, Ethyl-K, Lophophine, Substituted amphetamines, EDMA, Para-Methoxyamphetamine (PMA), Paramethoxymethamphetamine (PMMA), 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Substituted cathinones, Methylone, Ethylone, Eutylone, Butylone, Pentylone, 4-Ethyl methcathinone, 3-Methylmetheathinone, Substituted benzofurans, 5-AP B, 6-AP B, 5-APDB, 6-APDB, 5-MAPB, 5-MAPDB, 6-MAPDB, 6-MAPB, 5-EAPB, 6-EAPB, 5-MBPB, MDAT, MDMAT, 6-CAT, Tetralinylaminopropane, Trifluoromethylaminoindane, Ethyltrifluoromethylaminoindane, 5-Iodo-2-aminoindane, MMAI, MDAI, MDMAI, Indanylaminopropane, Naphthylaminopropane, 4-chlorophenylisobutylamine, 4-Methylphenylisobutylamine, Ariadne, α-methyltryptamine, 5-MeO-αMT, α-ethyltryptamine, 4-Me-αET, 7-Me-αET, 5-MeO-αET, 5-MeO-MiPT, Δ⁹-THC, CBD, CBN, THCV, (C6)-CP 47,497, (C9)-CP 47,497, 1-Butyl-3-(2-methoxybenzoyl)indole, 1-Butyl-3-(4-methoxybenzoyl)indole, 1-Pentyl-3-(2-methoxybenzoyl)indole, 2-Isopropyl-5-methyl-1-(2,6-di hydroxy-4-nonylphenyl)cyclohex-1-ene, 4-HTMPIPO, 4-Nonylphenylboronic acid, 5Br-U R-144, 5Cl-APINACA, 5C1-U R-144, 5F-3-pyridinoylindole, 5F-AB-FUPPYCA, 5F-ADB-PINACA, 5F-ADBICA, 5F-ADB, 5F-AMB, 5F-APINACA, 5F-CUMYL-PINACA, 5F-EMB-PINACA, 5F-NNE1, 5F-PB-22, 5F—PCN, 5F—PY-PICA, 5F—PY—P INACA, 5F-SDB-006, HHC, A-796,260, A-834,735, A-836,339, A-955,840, A-40174, A-41988, A-42574, AB-001, AB-CH FU PYCA, AB-CHMFUPPYCA, AB-CHMINACA, AB-FUBICA, AB-FUBINACA 2-fluorobenzyl isomer, AB-FUBINACA, AB-PICA, AB-PINACA, Abnormal cannabidiol, ADAMANTYL-THPINACA, ADB-CHMINACA, ADB-FUBICA, ADB-FUBINACA, ADB-PINACA, ADBICA, ADS B—FU B-187, Ajulemic acid, AM-087, AM-411, AM-630, AM-679, AM-694, AM-855, AM-883, AM-905, AM-906, AM-919, AM-926, AM-938, AM-1220, AM-1221, AM-1235, AM-1241, AM-1248, AM-1346, AM-1387, AM-1714, AM-2201, AM-2232, AM-2233, AM-2389, AM-4030, AM-4113, AM-6527, AM-6545, AM-251, AM-281, AM-404, AMB-CHMINACA, AMB-FUBINACA, AMG-1, AMG-3, AMG-36, AMG-41, APICA, APINACA, APP-FUBINACA, Arachidonoyl SEROTONIN, ACEA, ACPA, Arvanil, AZ-11713908, BAY 38-7271, BAY 59-3074, BIM-018, Biochanin A, BML-190, Nabidrox (Canbisol), Cannabicyclohexanol, Cannabipiperidiethanone, CAY-10401, CAY-10429, CAY-10508, CB-13, CB-25, CB-52, CB-86, CB-86, CBS-0550, CP 47,497, CP 55,244, CP 55,940, CUMYL-5F-PICA, CUMYL-BICA, CUMYL-PICA, CUMYL-PINACA, CUMYL-THPINACA, Dexanabinol, Dimethylheptylpyran, Drinabant, Dronabinol, EAM-2201, EMB-FUBINACA, FAB-144, FDU-NNE1, FDU-PB-22, FUB-144, FUB-APINACA, FUB-JWH-018, FUB—PB-22, FUBIMINA, Genistein, GW-405,833, GW-842,166X, Hemopressin, HU-210, HU-243, HU-308, HU-320, HU-331, HU-336, HU-345, HU-910, Ibipinabant, IDFP, JNJ 1661010, JNJ 1661010, JTE-907, JTE 7-31, JWH-007, JWH-015, JWH-018, JWH-019, JWH-030, JWH-051, JWH-073, JWH-081, JWH-098, JWH-116, JWH-122, JWH-133, JWH-139, JWH-147, JWH-149, JWH-161, JWH-164, JWH-167, JWH-175, JWH-176, JWH-182, JWH-184, JWH-185, JWH-192, JWH-193, JWH-194, JWH-195, JWH-196, JWH-197, JWH-198, JWH-199, JWH-200, JWH-203, JWH-210, JWH-229, JWH-249, JWH-250, JWH-251, JWH-302, JWH-307, JWH-359, JWH-369, JWH-370, JWH-398, JWH-424, JZL184, JZL195, Kaempferol, KM-233, L-759,633, L-759,656, LASSBio-881, LBP-1, Leelamine, Levonantradol, LH-21, LY-320,135, LY-2183240, NAM-2201, MDM-2201, MDA-7, MDA-19, MDA-77, MDMB-CHMICA, MDMB-CHMINACA, MDMB-FUBINACA, Menabitan, MEPIRAPIM, Methanandamide, MJ-15, MK-9470, MMB-2201, MN-18, MN-25, Nabazenil, Nabilone, Nabitan, Naboctate, NESS-0327, NESS-040C5, NIDA-41020, NM-2201, NMP-7, NNE1, Nonabine O-224, O-581, O-585, O-606, O-689, O-774, O-806, O-823, O-889, O-1057, O-1125, O-1184, O-1191, O-1238, O-1248, O-1269, O-1270, O-1376, O-1399, O-1422, O-1601, O-1602, O-1624, O-1656, O-1657, O-1660, O-1812, O-1860, O-1861, O-1871, O-1918, O-2048, O-2050, O-2093, O-2113, O-2220, O-2365, O-2372, O-2373, O-2383, O-2426, O-2484, O-2545, O-2654, O-2694, O-2715, O-2716, O-3223, O-3226, Oleoylethanolamide, Olvanil, Org 27569, Org 27759, Org 2831, Org 28611, Org 29647, Otenabant, Palmitoylethanolamide, Parahexyl, PF-03550096, PF-04457845, PF-622, PF-750, PF-3845, PF-514273, PHOP, PipISB, Pimabine, Pravadoline, Pregnenolone, PSB—SB-487, PSB—SB-1202, PTI-1, PTI-2, PX-1, PX-2, PX-3, QUCHIC, QUPIC, RCS-4, RCS-8, Rimonabant, Rosonabant, RTI-371, S-444,823, SDB-006, SER-601, SPA-229, SR-144,528, STS-135, Surinabant, Taranabant, Tedalinab, THC-O-acetate, THC-O-phosphate, THJ-018, THJ-2201, Tinabinol, TM-38837, UR-144, URB-447, URB-447, URB-597, URB-602, URB-754, VCHSR, VDM-11, VSN-16, WIN 54,461, WIN 55,212-2, WIN 56,098, XLR-11, Yangonin, Harmaline, harmala alkaloids, other beta-carbolines, active constituents of ayahuasca, Salvinorin A, Salvinorin B methoxymethyl ether, Salvinorin B ethoxymethyl ether, Piperazines, pFPP, TFMPP, Myristicin, Elemicin, Cryogenine (Vertine), Atropine, Scopolamine, Hyoscyamine, Ibotenic acid, Muscimol, TiHKAL, 2-Me-DET, 4-HO-DBT, 4-HO-DET, 4-HO-DiPT, 4-HO-EPT, 4-HO-McPT, 4-HO-MET, 4-HO-MiPT, 4-HO-MPMI, 4-HO-MPT, 4-HO-uMT, 4-Me-uMT, 4-MeO-MiPT, 4-PrO-DMT, 4,5-MDO-DiPT, 4,5-MDO-DMT, 5-Ethoxy-uMT, 5-Fluoro-AET, 5-Fluoro-DET, 5-Fluoro-DMT, 5-Fluoro-EPT, 5-Fluoro-MET, 5-MeO-AET, 5-MeO-aMT, 5-MeO-DALT, 5-MeO-DET, 5-MeO-DPT, 5-MeO-EiPT, 5-MeO-MALT, 5-MeO-MiPT, 5-MeO-MPMI, 5-MeO-pyr-T, 5-MeS-DMT, 5-MeO-2-TMT, 5-TFM-DMT, 5-TFMO-DMT, 5,6-MDO-DiPT, 5,6-MDO-DMT, 5,6-MDO-MiPT, 5,6-MeO-MiPT, 5,N,N-TMT, 5F-MPMI, 6-Fluoro-DET, 6-Fluoro-DMT, 6-MeO-THH, 7-Chloro-AMT, 7-F-5-MeO-MET, 4-Acetoxy-DET, 4-Acetoxy-DiPT, 4-Acetoxy-MET, 4-Acetoxy-MiPT, 0-Acetylbufotenine, 0-Acetylpsilocin, Aeruginascin, Alpha,N-DMT, Alpha,N,O-TMS, Baeocystin, 5-Bromo-DMT, Bufotenin, 5-Chloro-uMT, DALT, Dibutyltryptamine, Diethyltryptamine, Diisopropyltryptamine, 5,N-Dimethyl-N-isopropyltryptamine, Dimethyltryptamine, N,N-Dimethyltryptamine, Dipropyltryptamine, 2,α-Dimethyltryptamine, Ethocybin, Ethylisopropyltryptamine, A-Ethyltryptamine, 5-Fluoro-AMT, 4-Fluoro-5-methoxy-DMT, FT-104, 5-MeO-DMT, 5-Methoxy-N,N-diisopropyltryptamine, 4-Methyl-α-ethyltryptamine, N-Methyl-N-ethyltryptamine, Methylbutyltryptamine, Methylisopropyltryptamine, Alpha-Methylserotonin, Alpha-Methyltryptamine, N-Methyltryptamine, MPMI, Norbaeocystin, Propylisopropyltryptamine, 2,N,N-TMT, A,N,N-Trimethyltryptamine, Ketamine, Esketamine, Arketamine, Mitragynine, Yohimbine, and any combinations thereof.

In some embodiments, the serotonergic psychedelic drug is a tryptamine or an ergoline. In some embodiments, the serotonergic psychedelic drug comprises a tryptamine, or a salt, solvate, enantiomer, or diastereomer thereof. In some embodiments, the serotonergic psychedelic drug comprises an ergoline, or a salt, solvate, enantiomer, or diastereomer thereof. In some embodiments, the serotonergic psychedelic drug is psilocybin, or a salt, solvate, enantiomer, or diastereomer thereof.

In some embodiments, the PDE9 inhibitor is selected from the group consisting of aminophylline, pentoxifylline, theobromine, paraxanthine, PF-04447913, PF-04447943, PF-04447953, BAY 73-6691, E 2027, CRD 773, BI 409306, CRD 740, TT 00920, BAY 7081, FRM 16606, HUC1-288, WYQ-C36D, and any combinations thereof. In some embodiments, the PDE9 inhibitor is PF-04447943, or a derivative thereof, or a salt, solvate, enantiomer or diastereomer thereof.

The present disclosure also provides a pharmaceutical composition comprising a serotonergic psychedelic drug and PDE9 inhibitor, wherein the serotonergic psychedelic drug is not mescaline. In some embodiments, the serotonergic psychedelic drug and the PDE9 inhibitor are present in amounts sufficient to treat or prevent a neuropsychiatric disorder in a subject. In some embodiments, the PDE9 inhibitor prolongs at least one biological activity of the serotonergic psychedelic drug in the subject. In some embodiments, the PDE9 inhibitor mitigates at least one undesirable biological activity of the serotonergic psychedelic drug in the subject. In some embodiments, the PDE9 inhibitor reduces the abuse potential of the serotonergic psychedelic drug in the subject. In some embodiments, the pharmaceutical composition is formulated for administration by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous. In some embodiments, the serotonergic psychedelic drug is selected from the group consisting of Psilocin, Psilocybin, Bufotenin, Baeocystin, Aeruginascin, 5-MeO-DMT, N,N-Dimethyltryptamine (DMT), 5-Bromo-DMT, N-Methyl-N-ethyltryptamine (MET), N-Methyl-N-isopropyltryptamine (MiPT), N-Methyl-N-propyltryptamine (MPT), N,N-Diethyltryptamine (DET), N-Ethyl-N-isopropyltryptamine (EiPT), N-Methyl-N-butyltryptamine (MBT), N-Propyl-N-isopropyltryptamine (PiPT), N,N-Dipropyltryptamine (DPT), N,N-Diisopropyltryptamine (DiPT), N,N-Diallyltryptamine (DALT), N,N-Dibutyltryptamine (DBT), N-Ethyltryptamine (NET), N-Methyltryptamine (NMT), Trimethyltryptamine (TMT), α-Methyltryptamine, α-Ethyltryptamine, α,N-DMT, α,N,N-Trimethyltryptamine, Ethocybin, 4-HO-MET, 4-HO-DET, 4-HO-MPT, 4-HO-MiPT, 4-HO-MALT, 4-HO-DPT, 4-HO-DiPT, 4-HO-DALT, 4-HO-DBT, 4-HO-DSBT, 4-HO-uMT, 4-HO-MPMI, 4-HO-TMT, 4-HO-1,N,N-TMT, 4-HO-5-MeO-DMT, 4-AcO-DMT, 4-AcO-MET, 4-AcO-MALT, 4-AcO-DET, 4-AcO-EiPT, 4-AcO-DPT, 4-AcO-DiPT, 4-AcO-DALT, 4-MeO-DMT, 4-MeO-MiPT, 5-MeO—N MT, 5-MeO-MET, 5-MeO-MPT, 5-MeO-MiPT, 5-MeO-DET, 5-MeO-Ei PT, 5-MeO-EPT, 5-MeO-Pi PT, 5-MeO-DPT, 5-MeO-DiPT, 5-MeO-DALT, 5-MeO-uMT, 5-MeO-2,N,N-TMT, 5-MeO-7,N,N-TMT, 5-MeO-α,N-DMT, 4-F-5-MeO-DMT, 5-Me-MiPT, 5-HO-DiPT, 2-α-DMT, 4-Me-uMT, 4-Me-αET, 7-Me-αET, 4,5-DHP-AMT, 4,5-DH P-DMT, 4,5-MDO-DMT, 4,5-MDO-DiPT, 5,6-MDO-DiPT, 5,6-MDO-MiPT, 5-Fluoro-uMT, 6-Fluoro-uMT, 6-Fluoro-DMT, N,N-Tetramethyl enetryptamine (Pyr-T), 4-HO-pyr-T, 5-MeO-pyr-T, RU-28306 (4,α-Methylene-N,N-DMT), O-4310 (6-Fluoro-1-Isopropyl-4-HO-DMT), CP-132,484 (4,5-DHP-1-Methyltryptamine), Dimemebfe (5-MeO-BFE), 5-MeO-DiBF, Ibogaine, Voacangine, Lysergic acid diethylamide (LSD), Lysergic acid amide (LSA), Lysergic acid diamide, N1-Methyl-lysergic acid diethylamide, 1-Propionyl-lysergic acid diethylamide, 1-cyclopropanoyl-d-lysergic acid diethylamide, 1-valeryl-D-lysergic acid diethylamide, 6-Allyl-6-nor-lysergic acid diethylamide, 6-Butyl-6-nor-lysergic acid diethylamide, 6-Ethyl-6-nor-lysergic acid diethylamide, 1-Propionyl-6-Ethyl-6-nor-lysergic acid diethylamide, 6-Propyl-6-nor-lysergic acid diethylamide, 6-Cyclopropyl-6-nor-lysergic acid diethylamide, 6-nor-Lysergic acid diethylamide, Lysergic acid ethylamide, Lysergic acid α-hydroxyethylamide, Lysergic acid 2-butyl amide, Lysergic acid 3-pentyl amide, Lysergic acid methyl ester, Lysergic acid 2,4-dimethylazetidide, Lysergic acid piperidine, N,N-Dimethyl-lysergamide, Methylisopropyllysergamide, N,N-Diallyllysergamide, N-Pyrrolidyllysergamide, N-Morpholinyllysergamide, 1-methyl-lysergic acid butanolamide, Lysergic acid 0-propanolamide, Lysergic acid 1-butanolamide, Mescaline, Lophophine, Isomescaline, Cyclopropylmescaline, Thioisomescaline (2-TIM, 3-TIM, and 4-TIM), 4-Desoxymescaline, Jimscaline, Escaline, Metaescaline, Thiometaescaline (3-TME, 4-TME, and 5-TME), Trisescaline, Thiotrisescaline (3-T-TRIS and 4-T-TRIS), Symbescaline, Asymbescaline, Thiosymbescaline (3-TSB and 4-TSB), Phenescaline, Allylescaline, Methallylescaline, Proscaline, Isoproscaline, Metaproscaline, Thioproscaline, Buscaline, Thiobuscaline, α-ethylmescaline, Ariadne, Macromerine, MEPEA, TOM (2-TOM and 5-TOM), Bis-TOM, TOMSO (2-methoxy-4-methyl-5-methylsulfinylamphetamine), TOET (2-TOET and 5-TOET), BOH, BOM (13-Methoxy-mescaline), beta-D, 4-D, DME, F-2, F-22, FLEA, MDPH, MDMP, Propynyl, 2C family (2,5-dimethoxy, 4-substituted phenethylamines), Pk-2C-B, 2C—B, 2CB-2EtO, 2CB-5EtO, 2CB-diEtO, 2C—B-FLY, 2C—B-BUTTERFLY, 2C—C, 2C-D, 2CD-2EtO, 2CD-diEtO, 2CD-5EtO, 2C-E, 2C-EF, 2C—F, 2C-G (2C-G-1, 2C-G-2, 2C-G-3, 2C-G-4, 2C-G-5, 2C-G-6, and 2C-G-N), 2C—H, 2C—I, 2CI-2EtO, 2C-iP, 2C—N, 2C—O, 2C—O-4, 2C—P, 2C-SE, 2C-T, 2CT-5EtO, 2C-T-2, 2CT-2-2EtO, 2CT-2-5EtO, 2CT-2-diEtO, 2C-T-4 (2C-T-4 and Ψ-2C-T-4), 2CT-4-2EtO, 2C-T-7, 2CT-7-2EtO, 2C-T-8, 2C-T-9, 2C-T-13, 2C-T-15, 2C-T-16, 2C-T-17, 2C-T-19, 2C-T-21, 2C-TFM, 2C—YN, BOB (13-Methoxy-2C-B), BOD (13-Methoxy-2C-D), BOHD (13-Hydroxy-2C-D), HOT-2, HOT-7, HOT-17, Indane derivatives comprising 2CB-Ind, Benzocyclobutene derivatives comprising 2C—BCB (TCB-2), NBOMe derivatives comprising NBOMe-mescaline, 2C—H—NBOMe, 2C—C—NBOMe, 2CBCB—NBOMe, 2CBFly-NBOMe, 2C—B—NBOMe, 2C—I—NBOMe, 2C-TFM-NBOMe, 2C-D-NBOMe, 2C-G-NBOMe, 2C-E-NBOMe, 2C—P—NBOMe, 2C-iP-NBOMe, 2C—CN—NBOMe, 2C—N—NBOMe, 2C-T-NBOMe, 2C-T-4-NBOMe, 2C-T-7-NBOMe, and DMBMPP, NBOH derivatives comprising 2C—C—NBOH, 2C—B—NBOH, 2C—I—NBOH, and 2C—CN—NBOH, NBMD derivatives comprising 2C—I-NBMD, NBF derivatives comprising 2C—C—NBF, 2C—B—NBF, and 2C—I—NBF, 3C family (3,5-dimethoxy, 4-substituted amphetamines) comprising 3C-E, 3C—P, 3C-DFE, and 3C—BZ, DOx family (2,5-dimethoxy, 4-substituted amphetamines) comprising DOAM, DOB, Meta-DOB, Methyl-DOB, DOBU, DOC, DOEF, DOET, DOI, DOM, Ψ-DOM, DON, DOPR, SOiPR, DOT, Meta-DOT, Ortho-DOT, and DOTFM, DMCPA, DMMDA, DMMDA-2, 2,5-dimethoxy-3,4-dimethylamphetamine, 4-methyl-2,5-dimethoxymethamphetamine, 2,N-dimethyl-4,5-methylenedioxy amphetamine, Dimethoxyamphetamine (2,4-DMA, 2,5-DMA, and 3,4-DMA), Trimethoxy amphetamine (TMA-2, TMA-6), Tetramethoxyamphetamine, Br-DragonFLY, TFMFly, 2-Bromo-4,5-methylenedioxyamphetamine, 4-Bromo-3,5-dimethoxyamphetamine, EEE, EEM, EME, EMM, EDMA, EIDA, Ethyl-J, Methyl-J, Ethyl-K, Mehtyl-K, IDNNA, Iris, MDAI, MDMAI, MDAT, MDMAT, MDAL, MDBU, MDBZ, MDDM, MDIP, MDMEOET, MDMEO, MDOH, MDHOET, MDPL, MDCPK, MDPR, MEDA, MEM, Mehtyl-DMA, MMDA, MMDA-2, 5-Mehtyl-MDA, MEE, MME, MPM, DiFMDA, 5-APB, 6-APB, 5-APDB, 6-APDB, 5-MAPB, 5-MAPDB, 6-MAPDB, 6-MAPB, 6-EAPB, 5-EAPB, Para-Methoxyamphetamine, Paramethoxymethamphetamine, 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Benzoxazine, Efavirenz, Substituted methylenedioxy-phenethylamines (MDxx) comprising 3,4-methylenedioxymethamphetamine (MDMA), MDA, 2,3-MDA, 5-Methyl-MDA, MMDA, MDEA, MBDB, MDAL, MDBU, MDBZ, MDDM, MDIP, MDMEOET, MDMEO, MDOH, MDHOET, MDPL, MDCPM, MDPR, BDB, MMDA-2, DiFMDA, EIDA, Ethyl-K, Lophophine, Substituted amphetamines, EDMA, Para-Methoxyamphetamine (PMA), Paramethoxymethamphetamine (PMMA), 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Substituted cathinones, Methylone, Ethylone, Eutylone, Butylone, Pentylone, 4-Ethyl methcathinone, 3-Methylmethcathinone, Substituted benzofurans, 5-AP B, 6-AP B, 5-APDB, 6-APDB, 5-MAPB, 5-MAPDB, 6-MAPDB, 6-MAPB, 5-EAPB, 6-EAPB, 5-MBPB, MDAT, MDMAT, 6-CAT, Tetralinylaminopropane, Trifluoromethylaminoindane, Ethyltrifluoromethylaminoindane, 5-Iodo-2-aminoindane, MMAI, MDAI, MDMAI, Indanylaminopropane, Naphthylaminopropane, 4-chlorophenylisobutylamine, 4-Methylphenylisobutylamine, Ariadne, α-methyltryptamine, 5-MeO-uMT, α-ethyltryptamine, 4-Me-αET, 7-Me-αET, 5-MeO-αET, 5-MeO-MiPT, Δ⁹-THC, CBD, CBN, THCV, (C6)-CP 47,497, (C9)-CP 47,497, 1-Butyl-3-(2-methoxybenzoyl)indole, 1-Butyl-3-(4-methoxybenzoyl)indole, 1-Pentyl-3-(2-methoxybenzoyl)indole, 2-Isopropyl-5-methyl-1-(2,6-di hydroxy-4-nonylphenyl)cyclohex-1-ene, 4-HTMPIPO, 4-Nonylphenylboronic acid, 5Br—U R-144, 5Cl-APINACA, 5Cl—U R-144, 5F-3-pyridinoylindole, 5F-AB-FUPPYCA, 5F-ADB-PINACA, 5F-ADBICA, 5F-ADB, 5F-AMB, 5F-APINACA, 5F-CUMYL-PINACA, 5F-EMB-PINACA, 5F-NNE1, 5F—PB-22, 5F—PCN, 5F—PY-PICA, 5F—PY—P INACA, 5F-SDB-006, HHC, A-796,260, A-834,735, A-836,339, A-955,840, A-40174, A-41988, A-42574, AB-001, AB-CH FU PYCA, AB-CHMFUPPYCA, AB-CHMINACA, AB-FUBICA, AB-FUBINACA 2-fluorobenzyl isomer, AB-FUBINACA, AB-PICA, AB-PINACA, Abnormal cannabidiol, ADAMANTYL-THPINACA, ADB-CHMINACA, ADB-FUBICA, ADB-FUBINACA, ADB-PINACA, ADBICA, ADS B—FU B-187, Ajulemic acid, AM-087, AM-411, AM-630, AM-679, AM-694, AM-855, AM-883, AM-905, AM-906, AM-919, AM-926, AM-938, AM-1220, AM-1221, AM-1235, AM-1241, AM-1248, AM-1346, AM-1387, AM-1714, AM-2201, AM-2232, AM-2233, AM-2389, AM-4030, AM-4113, AM-6527, AM-6545, AM-251, AM-281, AM-404, AMB-CHMINACA, AMB-FUBINACA, AMG-1, AMG-3, AMG-36, AMG-41, APICA, APINACA, APP-FUBINACA, Arachidonoyl SEROTONIN, ACEA, ACPA, Arvanil, AZ-11713908, BAY 38-7271, BAY 59-3074, BIM-018, Biochanin A, BML-190, Nabidrox (Canbisol), Cannabicyclohexanol, Cannabipiperidiethanone, CAY-10401, CAY-10429, CAY-10508, CB-13, CB-25, CB-52, CB-86, CB-86, CBS-0550, CP 47,497, CP 55,244, CP 55,940, CUMYL-5F-PICA, CUMYL-BICA, CUMYL-PICA, CUMYL-PINACA, CUMYL-THPINACA, Dexanabinol, Dimethylheptylpyran, Drinabant, Dronabinol, EAM-2201, EMB-FUBINACA, FAB-144, FDU-NNE1, FDU-PB-22, FUB-144, FUB-APINACA, FUB-JWH-018, FUB—PB-22, FUBIMINA, Genistein, GW-405,833, GW-842,166X, Hemopressin, HU-210, HU-243, HU-308, HU-320, HU-331, HU-336, HU-345, HU-910, Ibipinabant, IDFP, JNJ 1661010, JNJ 1661010, JTE-907, JTE 7-31, JWH-007, JWH-015, JWH-018, JWH-019, JWH-030, JWH-051, JWH-073, JWH-081, JWH-098, JWH-116, JWH-122, JWH-133, JWH-139, JWH-147, JWH-149, JWH-161, JWH-164, JWH-167, JWH-175, JWH-176, JWH-182, JWH-184, JWH-185, JWH-192, JWH-193, JWH-194, JWH-195, JWH-196, JWH-197, JWH-198, JWH-199, JWH-200, JWH-203, JWH-210, JWH-229, JWH-249, JWH-250, JWH-251, JWH-302, JWH-307, JWH-359, JWH-369, JWH-370, JWH-398, JWH-424, JZL184, JZL195, Kaempferol, KM-233, L-759,633, L-759,656, LASSBio-881, LBP-1, Leelamine, Levonantradol, LH-21, LY-320,135, LY-2183240, NAM-2201, MDM-2201, MDA-7, MDA-19, MDA-77, MDMB-CHMICA, MDMB-CHMINACA, MDMB-FUBINACA, Menabitan, MEPIRAPIM, Methanandamide, MJ-15, MK-9470, MMB-2201, MN-18, MN-25, Nabazenil, Nabilone, Nabitan, Naboctate, NESS-0327, NESS-040C5, NIDA-41020, NM-2201, NMP-7, NNE1, Nonabine O-224, O-581, O-585, O-606, O-689, O-774, O-806, O-823, O-889, O-1057, O-1125, O-1184, O-1191, O-1238, O-1248, O-1269, O-1270, O-1376, O-1399, O-1422, O-1601, O-1602, O-1624, O-1656, O-1657, O-1660, O-1812, O-1860, O-1861, O-1871, O-1918, O-2048, O-2050, O-2093, O-2113, O-2220, O-2365, O-2372, O-2373, O-2383, O-2426, O-2484, O-2545, O-2654, O-2694, O-2715, O-2716, O-3223, O-3226, Oleoylethanolamide, Olvanil, Org 27569, Org 27759, Org 2831, Org 28611, Org 29647, Otenabant, Palmitoylethanolamide, Parahexyl, PF-03550096, PF-04457845, PF-622, PF-750, PF-3845, PF-514273, PHOP, PipISB, Pimabine, Pravadoline, Pregnenolone, PSB—SB-487, PSB—SB-1202, PTI-1, PTI-2, PX-1, PX-2, PX-3, QUCHIC, QUPIC, RCS-4, RCS-8, Rimonabant, Rosonabant, RTI-371, S-444,823, SDB-006, SER-601, SPA-229, SR-144,528, STS-135, Surinabant, Taranabant, Tedalinab, THC-O-acetate, THC-O-phosphate, THJ-018, THJ-2201, Tinabinol, TM-38837, UR-144, URB-447, URB-447, URB-597, URB-602, URB-754, VCHSR, VDM-11, VSN-16, WIN 54,461, WIN 55,212-2, WIN 56,098, XLR-11, Yangonin, Harmaline, harmala alkaloids, other beta-carbolines, active constituents of ayahuasca, Salvinorin A, Salvinorin B methoxymethyl ether, Salvinorin B ethoxymethyl ether, Piperazines, pFPP, TFMPP, Myristicin, Elemicin, Cryogenine (Vertine), Atropine, Scopolamine, Hyoscyamine, Ibotenic acid, Muscimol, TiHKAL, 2-Me-DET, 4-HO-DBT, 4-HO-DET, 4-HO-DiPT, 4-HO-EPT, 4-HO-McPT, 4-HO-MET, 4-HO-MiPT, 4-HO-MPMI, 4-HO-MPT, 4-HO-uMT, 4-Me-uMT, 4-MeO-MiPT, 4-PrO-DMT, 4,5-MDO-DiPT, 4,5-MDO-DMT, 5-Ethoxy-uMT, 5-Fluoro-AET, 5-Fluoro-DET, 5-Fluoro-DMT, 5-Fluoro-EPT, 5-Fluoro-MET, 5-MeO-AET, 5-MeO-aMT, 5-MeO-DALT, 5-MeO-DET, 5-MeO-DPT, 5-MeO-EiPT, 5-MeO-MALT, 5-MeO-MiPT, 5-MeO-MPMI, 5-MeO-pyr-T, 5-MeS-DMT, 5-MeO-2-TMT, 5-TFM-DMT, 5-TFMO-DMT, 5,6-MDO-DiPT, 5,6-MDO-DMT, 5,6-MDO-MiPT, 5,6-MeO-MiPT, 5,N,N-TMT, 5F-MPMI, 6-Fluoro-DET, 6-Fluoro-DMT, 6-MeO-THH, 7-Chloro-AMT, 7-F-5-MeO-MET, 4-Acetoxy-DET, 4-Acetoxy-DiPT, 4-Acetoxy-MET, 4-Acetoxy-MiPT, 0-Acetylbufotenine, O-Acetylpsilocin, Aeruginascin, Alpha,N-DMT, Alpha,N,O-TMS, Baeocystin, 5-Bromo-DMT, Bufotenin, 5-Chloro-uMT, DALT, Dibutyltryptamine, Diethyltryptamine, Diisopropyltryptamine, 5,N-Dimethyl-N-isopropyltryptamine, Dimethyltryptamine, N,N-Dimethyltryptamine, Dipropyltryptamine, 2,α-Dimethyltryptamine, Ethocybin, Ethylisopropyltryptamine, A-Ethyltryptamine, 5-Fluoro-AMT, 4-Fluoro-5-methoxy-DMT, FT-104, 5-MeO-DMT, 5-Methoxy-N,N-diisopropyltryptamine, 4-Methyl-α-ethyltryptamine, N-Methyl-N-ethyltryptamine, Methylbutyltryptamine, Methylisopropyltryptamine, Alpha-Methylserotonin, Alpha-Methyltryptamine, N-Methyltryptamine, MPMI, Norbaeocystin, Propylisopropyltryptamine, 2,N,N-TMT, A,N,N-Trimethyltryptamine, Ketamine, Esketamine, Arketamine, Mitragynine, Yohimbine, and any combinations thereof.

In some embodiments, the serotonergic psychedelic drug is a tryptamine or an ergoline. In some embodiments, the serotonergic psychedelic drug comprises a tryptamine, derivatives or salts thereof. In some embodiments, the serotonergic psychedelic drug comprises an ergoline, derivatives or salts thereof. In some embodiments, the serotonergic psychedelic drug is psilocybin, derivatives or salts thereof.

In some embodiments, the PDE9 inhibitor is selected from the group consisting of aminophylline, pentoxifylline, theobromine, paraxanthine, PF-04447913, PF-04447943, PF-04447953, BAY 73-6691, E 2027, CRD 773, BI 409306, CRD 740, TT 00920, BAY 7081, FRM 16606, HUC1-288, WYQ-C36D, and any combinations thereof. In some embodiments, the PDE9 inhibitor is PF-04447943, or a derivative thereof, or a salt, solvate, enantiomer or diastereomer thereof.

Provided herein is a kit comprising the pharmaceutical composition described hereof, wherein the kit further comprises an applicator and an instructional material for use thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (“Figure.” or “Figures.” herein), of which:

FIG. 1 presents Psilocybin head twitch response (HTR) reduction by PDE9 inhibitor in a dose-dependent manner.

FIG. 2 presents HTR reduction by PDE9 inhibitor in a psilocybin dose-dependent manner.

### DETAILED DESCRIPTION

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, illustrative methods and materials are described. As used herein, each of the following terms has the meaning associated with it in this section.

Generally, the nomenclature used herein and the laboratory procedures in molecular biology, pharmacology and organic chemistry are those well-known and commonly employed in the art.

Standard techniques are used for biochemical and/or biological manipulations. The techniques and procedures are generally performed according to conventional methods in the art and various general references (e.g., Sambrook & Russell, 2012, Molecular Cloning, A Laboratory Approach, Cold Spring Harbor Press, Cold Spring Harbor, NY, and Ausubel, et al, 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or +10%, more preferably ±5%, even more preferably +1%, and still more preferably +0.1% from the specified value, as such variations are appropriate to perform the disclosed methods.

The term “exemplary” as used herein means “serving as an example, instance, or illustration.” Any embodiment described herein as “exemplary” is not to be construed as preferred or advantageous over other embodiments.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

A disease or disorder is “alleviated” if the severity or frequency of at least one sign or symptom of the disease or disorder experienced by a patient is reduced.

“MDD” refers to major depressive disorder. “OCD” refers to obsessive-compulsive disorder. “PTSD” refers to post-traumatic stress disorder.

An “effective amount” or “therapeutically effective amount” of a compound or composition is that amount of compound or composition that is sufficient to provide a beneficial effect to the subject to which the compound or composition is administered, e.g., to achieve a therapeutic result, e.g., treatment of a certain disease or disorder. An “effective amount” of a delivery vehicle is that amount sufficient to effectively bind or deliver a compound or composition.

A “therapeutic effect” can be reduction and/or alleviation of the signs, symptoms, or causes of a disease or disorder, or any other desired alteration of a biological system.

The phrase “inhibit,” as used herein, means to reduce a molecule, a reaction, an interaction, a gene, an mRNA, and/or a protein's expression, stability, function, or activity by a measurable amount. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Naturally occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or composition useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound or composition useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound or composition useful within the present disclosure, and are physiologically acceptable to the patient. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example, in Remington's Pharmaceutical Sciences (Genaro, Ed., Mack Publishing Co., 1985, Easton, PA), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

A “therapeutic” treatment is a treatment administered to a subject who exhibits signs of pathology, for the purpose of diminishing or eliminating those signs.

As used herein, “treating a disease or disorder” means reducing the frequency and/or intensity with which a symptom of the disease or disorder is experienced by a patient. Disease and disorder are used interchangeably herein. Treatment encompasses prophylaxis and/or therapy. Accordingly the compositions and methods of the present disclosure are not limited to therapeutic applications and can be used in prophylaxis ones. Thus “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “psychedelic” or “psychedelic drug” or “psychedelic agent” refers to any of several pharmacological agents that cause acute and dramatic alterations in sensation and consciousness when administered to a human. The term “serotonergic psychedelic” refers to a psychedelic drug that acts at least in part by binding to serotonin receptors, especially the 5-HT2A serotonin receptor. Non-limiting examples of serotonergic psychedelics include 5-MeO-DMT and psilocybin.

As used herein, the term “NMDA” refers to N-methyl-D-aspartate.

As used herein, the term “NMDAR” or “NMDA-R” refers to an NMDA receptor. The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

As used herein, “serotonin”, “5-hydroxytryptamine”, and “5-HT”, which are synonymous and used interchangeably, refers to the molecule 5-hydroxytryptamine, which is, among other characteristics, a major modulatory neurotransmitter in the brain.

As used herein, “5-HT receptors” or “5-HTR” refers to any of several proteins in the brain to which serotonin (5-HT) binds to exert modulatory effects on neurons, and the corresponding genes that encode these proteins. 5-HT receptors may be further specified as, for example, “5-HT1A” for the serotonin type TA receptor, “5-HT1B” for the serotonin type 1B receptor, “5-HT2A” for the serotonin type 2A receptor, and so forth. The 5-HT2A receptor is thought to play a particular role in the action of serotonergic psychedelics.

As used herein, “LSD” or “lysergic acid diamide”, which are synonymous and used interchangeably, refers to the paradigmatic psychedelic drug lysergic acid diamide. “DMT” or “dimethyltryptamine”, which are synonymous and used interchangeably, refers to the psychedelic drug N,N-dimethyltryptamine, a relatively short-acting serotonergic psychedelic. “5-MeO-DMT” or “5-methoxydimethyltryptamine”, which are synonymous and used interchangeably, refers to the psychedelic drug 5-methoxy-N,N-dimethyltryptamine, a relatively short-acting serotonergic psychedelic.

As used herein, “RAAD” refers to a rapidly acting antidepressant.

As used herein, “cyclic adenosine monophosphate”, “cyclic AMP”, and “cAMP”, which are synonymous and used interchangeably, refer to cyclic adenosine monophosphate, a small molecule often used to mediate signaling processes within neurons and other cells in the body. As used herein, “cyclic guanine monophosphate”, “cyclic GMP”, and “cGMP”, which are synonymous and used interchangeably, refer to cyclic guanine monophosphate, another small molecule often used to mediate signaling processes within neurons and other cells in the body.

As used herein, “phosphodiesterase” or “PDE”, which are synonymous and used interchangeably, refer to any of several proteins whose primary function is to break down cAMP and/or cGMP, thereby modulating signaling processes within neurons or other cells in the body. These may be further specified as “phosphodiesterase type 1” or “PDE1”, “phosphodiesterase TA” or “PDE1A”, “phosphodiesterase type 9” or “PDE9”, “phosphodiesterase type 9A” or “PDE9A”, and so forth to indicate specific subtypes of phosphodiesterase protein, or the genes that encode them.

### Combination Treatment Therapy

Provided herein is the use of serotonergic psychedelic drugs as RAAD. Non-limiting examples of serotonergic psychedelics include psilocybin, mescaline, lysergic acid diamide (LSD), dimethyl tryptamine (DMT), 3,4-methylenedioxymethamphetamine (MDMA), and 5-methoxy-dimethyltryptamine (5-MeO-DMT). In some cases, these serotonergic psychedelic drugs can act as RAADs, and may be of benefit in other neuropsychiatric conditions, including (as non-limiting examples) PTSD, OCD, and addictive disorders. In some cases, these serotonergic psychedelic drugs produce profound changes in perception, thought, and emotion (‘psychedelic effects’) that last much longer than those of ketamine.

Inhibition of type-9 phosphodiesterase (PDE9) can reduce certain markers of psychedelic effects of serotonergic psychedelic drugs in animal models. In some embodiments of the present disclosure, PDE9 inhibition blocks psychedelic effects of these drugs without attenuating their therapeutic effects. In certain embodiments, the PDE9 inhibitor reduces undesirable psychedelic effects of serotonergic psychedelic drug, rendering it more appropriate for widespread use. In other embodiments, the PDE9 inhibitor prolongs the clinical effect(s) of the serotonergic psychedelic drug. In other embodiments, the phosphodiesterase inhibitor reduces the abuse liability of the serotonergic psychedelic drug.

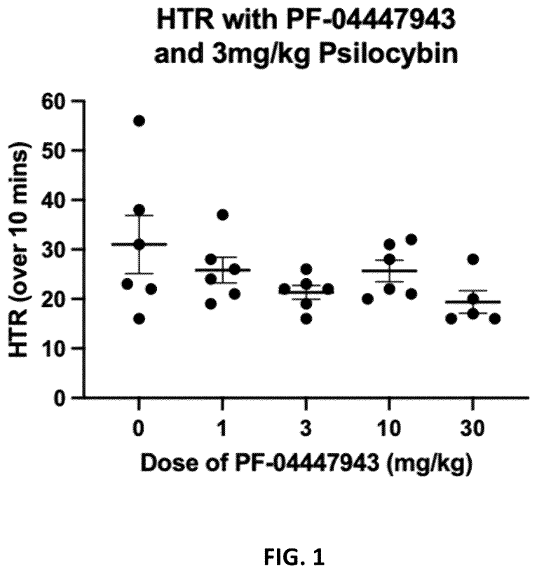

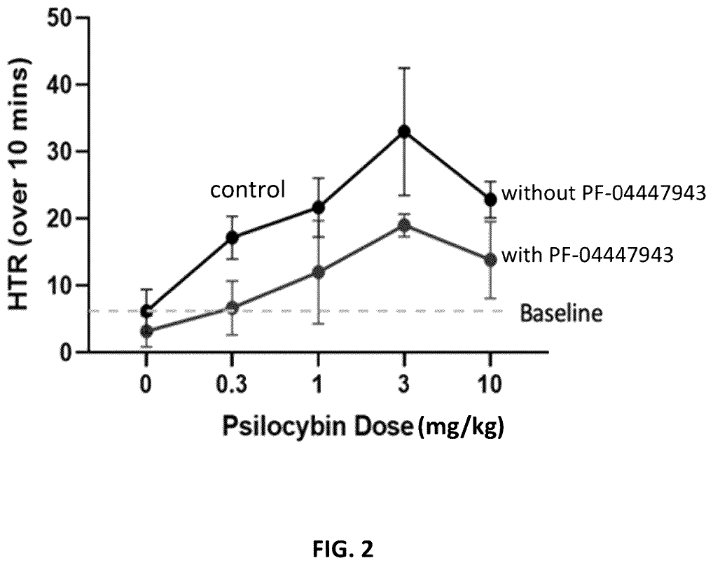

The studies presented herein (e.g., Example 1, FIG. 1, and FIG. 2) reveal multiple modes whereby PDE inhibition can mitigate undesirable side effects of RAADs, and produce synergistic beneficial effects on behavior and neurophysiology. Without wishing to be bound by a certain theory, by mitigating the undesirable side effects of serotonergic psychedelic drug treatment without disrupting benefit for the symptoms of MDD, PTSD, OCD, or other psychiatric conditions, and even enhancing aspects of them, concurrent PDE9 inhibition can enhance tolerability of serotonergic psychedelic drug treatment, permit treatment with reduced oversight and monitoring, reduce abuse potential for those serotonergic psychedelic drugs in which abuse potential is a limiting factor, and perhaps reduce frequency of dosing, thereby reducing cost, increasing the number of patients who can be treated, and reducing toxicity.

### PDE9 Inhibitors

Any PDE9 inhibitor known in the art is contemplated within the present application.

In some cases, non-limiting examples of PDE9 inhibitors include AKP-002 (ASP-4901), CRD-733, BAY-73-6691, Osoresnontrine (BI-409306), Irsenontrine (E-2027), Tovinontrine (IMR-687), and TT00920, and stereoisomers, pharmaceutically acceptable salts, solvates or prodrugs thereof.

In some cases, as disclosed in WO2020/076752, examples of PDE9 inhibitors include, but are not limited to, 1-{[2-(3-isopropyl-7-oxo-6,7-dihydro-1H-pyrazolo[4,3-d]pyrimidin-5-ylmethyl)-phenoxy]-acetyl}-pyrrolidine-2-carbo-xylic acid; 1-{[2-(1-cyclopentyl-4-oxo-4,5-dihydro-1H-pyrazolo[3,4-d]pyrim-idin-6-ylmethyl)-phenoxy]-acetyl}-pynOlidine-2(S)-carboxylic acid 3-isopropyl-5-[2-(2-oxo-2-piperazin-1-yl-ethoxy)-benzyl]-1,6-dihydro-pyrazolo[4,3-d]pyrimidin-7-one; 1-cyclopentyl-6-[2-(2-oxo-2-piperazin-1-yl-eth-oxy)-benzyl]-1,5-dihydro-pyrazolo[3,4-d]pyrimidin-4-one 3-isopropyl-5-[2-(2-morpholin-4-yl-2-oxo-ethoxy)-benzyl]-1,6-dihydro-pyra-zolo[4,3-d]pyrimidin-7-one; 3-isopropyl-5-[2-(2-oxo-2-pyrrolidin-1-yl-etho-xy)-benzyl]-1,6-dihydro-pyrazolo[4,3-d]pyrimidin-7-one; 5-{2-[2-(4-ethyl-piperazin-1-yl)-2-oxo-ethoxy]-benzyl}-3-isopropyl-1,6-di-hydro-pyrazolo[4,3-d]pyrimidin-7-one; N,N-diethyl-2-[2-(3-isopropyl-7-oxo˜6,7-dihydro-1H-pyrazolo[4,3-d]pyrimidin-5-ylmethyl)-phenoxy]-acetamide; 1-{[2-(3-isopropyl-7-oxo-6,7-dihydro-1H-pyrazolo[4,3-d]pyrimidin-5-ylmeth-yl)-phenoxy]-acetyl}-pyrrolidine-2-carboxy lie acid methyl ester; 4-{[2-(3-isopropyl-7-oxo-6,7-dihydro-1H-pyrazolo[4,3-d]pyrimidin-5-ylmeth-yl)-phenoxy]-acetyl}-piperazine-1-carboxylic acid tert-butyl ester; N-(2-dimethylamino-ethyl)-2-[2-(3-isopropyl-7-oxo-6,7-dihydro-1H-pyrazolo-[4,3-d]pyrimidin-5-ylmethyl)-phenoxy]-acetamide; 1-{[2-(1-cyclopentyl-4-ox-o-4,5-dihydro-1H-pyrazolo[3,4-d]pyrimidin-6-ylmethyl)-phenoxy]-acetyl}-pyr-rolidine-2-carboxylic acid methyl ester; 4-{[2-(1-cyclopentyl-4-oxo-4,5-di-hydro-1H-pyrazolo[3,4-d]pyrimidin-6-ylmethyl)-phenoxy]-acetyl}-piperazine-1-carboxylic acid tert-butyl ester; l-cyclopentyl-6-[2-(2-oxo-2-pyrrolidin-1-yl-ethoxy)-benzyl]-1,5-dihydro-pyrazolo[3,4-d]pyrimidin-4-one; 1-cyclopentyl-6-[2-(2-morpholin-4-yl-2-oxo-ethoxy)-benzyl]-1,5-dihydro-pyrazolo[3,4-d]pyrimidin-4-one; 2-[2-(1-cyclopentyl-4-oxo-4,5-dihydro-1H-pyr-azolo[3,4-d]pyrimidin-6-ylmethyl)-phenoxy]-N-(2-dimethylamino-ethyl)-aceta-mide; 1-cyclopentyl-6-{2-[2-(4-ethyl-piperazin-1-yl)-2-oxo-ethoxy]-benzyl}-1,5-dihydro-pyrazolo[3,4-d]pyrimidin-4-one; 2-[2-(1-cyclopentyl-4-oxo-4,5-dihydro-1H-pyrazolo[3,4-d]pyrimidin-6-ylmethyl)-phenoxy]-N,N-diethyl-acet-amide; [2-(3-isopropyl-7-oxo-6, 7-dihydro-1H-pyrazolo[4,3-d]pyrimidin-5-ylm-ethyl)-phenoxy]-acetic acid; [2-(1-cyclopentyl-4-oxo-4,5-dihydro-1H-pyrazo-lo[3,4-d]pyrimidin-6-ylmethyl)-phenoxy]-acetic acid; 3-isopropyl-5-[2-(5-chloro-2-morpholin-4-yl-ethoxy)-benzyl]-1,6-dihydro-p-yrazolo[4,3-d]pyrimidin-7-one; 3-isopropyl-5-[2-(2-pyrrolidin-1-yl-ethoxy)-benzyl]-1,6-dihydro-pyrazolo[4,3-d]pyrimidin-7-one; 3-isopropyl-5-[2-(2-morpholin-4-yl-ethoxy)-cyclohexylmethyl]-1, 6-dihydro-pyrazolo[4,3-d]pyrimidin-7-one; 5-[5-fluoro-2-(2-morpholin-4-yl-ethoxy)-be-nzyl]-3-isopropyl-1,6-dihydro-pyrazolo[4,3-d]pyrimidin-7-one; 3-cyclopentyl-5-[5-fluoro-2-(2-morpholin-4-yl-ethoxy)-benzyl]-1, 6-dihydro-1-pyrazolo[4,3-d]pyrimidin-7-one; 3-isopropyl-5-[2-(2-morpholin-4-yl-ethoxy-)-benzyl]-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 2-[2-(2-morpholin-4-yl-ethoxy)-benzyl]-9-(tetrahydro-furan-3-yl)-1,9-dihydro-purin-6-one; 5-[2-(2-diethylamino-ethoxy)-benzyl]-3-isop-ropyl-1,6-dihydro-pyrazolo[4,3-d]pyrimidin-7-one; 3-cyclopentyl-5-[2-(2-mo-rpholin-4-yl-ethoxy)-benzyl]-1,6-dihydro-pyrazolo[4,3-d]pyrimidin-7-one; 3-cyclobutyl-5-[2-(2-morpholin-4-yl-ethoxy)-benzyl]-1,6-dihydro-pyrazolo[-4,3-d]pyrimidin-7-one; 9-(1(R),2-dimethyl propyl)-[2-(2-morpholin-4-yl-eth-oxy)-benzyl]-1,9-dihydro-purin-6-one; 9-(2-methyl-butyl)-2-[2-(2-morpholin-4-yl-ethoxy)-benzyl]-1,9-dihydro-purin-6-one; 9-cyclopentyl-2-[2-(2-morph-olin-4-yl-ethoxy)-benzyl]-1,9-dihydro-purin-6-one; 5-[2-(2-morpholin-4-yl-ethoxy)-benzyl]-3-pyridin-3-yl-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 9-isopropyl-2-[2-(2-morpholin-4-yl-ethoxy)-benzyl]-1,9-dihy-dro-purin-6-one; 2-[2-(2-morpholin-4-yl-ethoxy)-benzyl]-9-(tetrahy dro-fura-n-2-ylmethyl)-1,9-dihydro-purin-6-one; 9-(1-isopropyl-2-methyl-propyl)-2-[-2-(2-morpholin-4-yl-ethoxy)-benzyl]-1,9-dihydro-purin-6-one; 9-(1-ethyl-propyl)-2-[2-(2-morphobn-4-yl-ethoxy)-benzyl]-1, 9-dihydro-pur-in-6-one; 9-cyclopentyl-8-methyl-2-[2-(2-morphobn-4-yl-ethoxy)-benzyl]-1,-9-dihydro-purin-6-one; 3-cyclopentyl-5-[2-(2-morphobn-4-yl-ethoxy)-benzyl-]-3,6-dihydro-[1,2,3]triazolo[4,5-d]pyrimidin-7-one; 1-cyclopentyl-6-[2-(2-morpholin-4-yl-ethoxy)-benzyl]-1,5-dihydro-pyrazolo-[3,4-d]pyrimidin-4-one; 9-cyclopentyl-2-[2-(3-morpholin-4-yl-propoxy)-benz-yl]-1,9-dihydro-purin-6-one; N-[(lR,2S)₂-(3-isopropyl-7-oxo-6,7-dihydro-1H-pyrazolo[4,3-d]pyrimidin-5-ylmethyl)-cyclohex-1-yl]-2-pyrrobdin-1-yl-acetamide; N-[(1R,2S)2-(3-isopropyl-7-oxo-6,7-dihydro-1H-pyrazolo[4,3-d]pyrim-idin-5-ylmethyl)-cyclohex-1-yl]-2-morphobn-4-yl-acetamide; 2-diethylamino-N-[(lR,2S)2-(3-isopropyl-7-oxo-6,7-dihydro-1H-pyrazolo[4,3-d]pyrimidin-5-ylmethyl)-cyclohex-1-yl]-acetamide; 1-{[(lR,2S)2-(3-isoprop-yl-7-oxo-6,7-dihydro-1H-pyrazolo[4,3-d]pyrimidin-5-ylmethyl)-cyclohex-1-yl-carbamoyl]-methyl}-pyrrobdine-2(S)-carboxy lie acid methyl ester; 2-cyclobutylamino-N-[(lR,2S)2-(3-isopropyl-7-oxo-6,7-dihydro-1H-pyrazolo[-4,3-d]pyrimidin-5-ylmethyl)-cyclohex-1-yl]-acetamide; or 2-cyclopropylamino-N-[(lR,2S)2-(3-isopropyl-7-oxo-6,7-dihydro-1H-pyrazolo-[4,3-d]pyrimidin-5-ylmethyl)-cyclohex-1-yl]-acetamide; IMR-687, (a potent inhibitor of PDE9A), BAY73-6691, PF-04447943, PF—4181366, and stereoisomers, pharmaceutically acceptable salts, solvates and prodrugs thereof, and any combinations thereof.

In some cases, as disclosed in U.S. Pat. No. 9,573,947 and WO2014163147, PDE9 inhibitors include one or more of salts of pyrazoloquinoline derivatives as below:
[1] a salt of (S)-7-(2-methoxy-3,5-dimethylpyridin-4-yl)-1-(tetrahydrofuran-3-yl)-1H-pyrazolo[4,3-c]quinolin-4(5H)-one and an acid selected from the group consisting of hydrochloric acid, hydrobromic acid, sulfuric acid, nitric acid, phosphoric acid, malonic acid, maleic acid, tartaric acid, methanesulfonic acid, benzenesulfonic acid and toluenesulfonic acid;
[2] (S)-7-(2-methoxy-3,5-dimethylpyridin-4-yl)-1-(tetrahydrofuran-3-yl)-1H-pyrazolo[4,3-c]quinolin-4(5H)-one monomaleate salt;
[3] (S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt;
[4] a crystal of the salt according to [1];
[5] a crystal of (S)-7-(2-methoxy-3,5-dimethylpyridin-4-yl)-1-(tetrahydrofuran-3-yl)-1H-pyrazolo[4,3-c]quinolin-4(5H)-one monomaleate salt, having a diffraction peak at a diffraction angle (2θ±0.2°) of 10.1° in powder X-ray diffraction;
[6] a crystal of (S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt, having a diffraction peak at a diffraction angle (2θ±0.2°) of 9.9° in powder X-ray diffraction.

In some cases, as disclosed in WO2017/070293, PDE9 inhibitor is selected from the group consisting of:

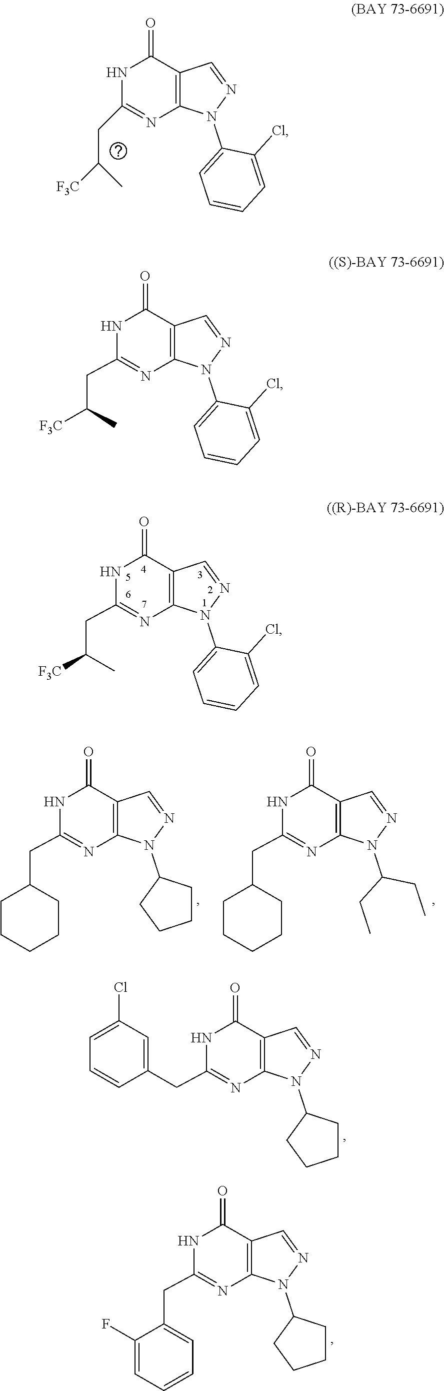

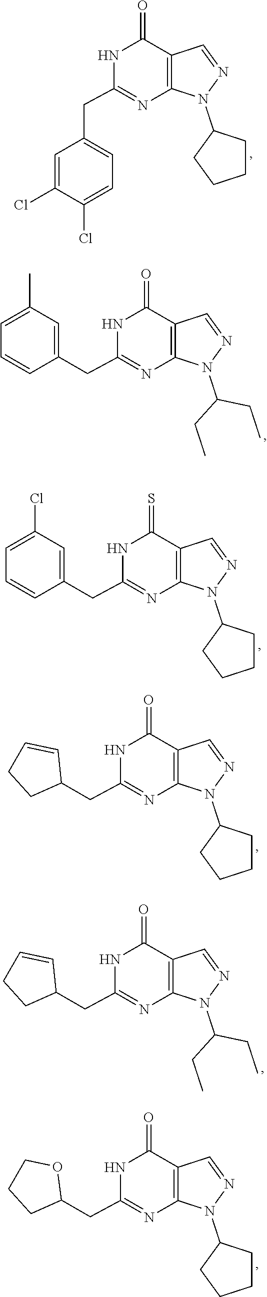 [SMILES: O=c1[nH]c(Cc2ccc(Cl)c(Cl)c2)nc2c1cnn2C1CCCC1 ; CCC(CC)n1ncc2c(=O)[nH]c(Cc3cccc(C)c3)nc21 ; S=c1[nH]c(Cc2cccc(Cl)c2)nc2c1cnn2C1CCCC1 ; O=c1[nH]c(CC2C=CCC2)nc2c1cnn2C1CCCC1 ; CCC(CC)n1ncc2c(=O)[nH]c(CC3C=CCC3)nc21 ; O=c1[nH]c(CC2CCCO2)nc2c1cnn2C1CCCC1]

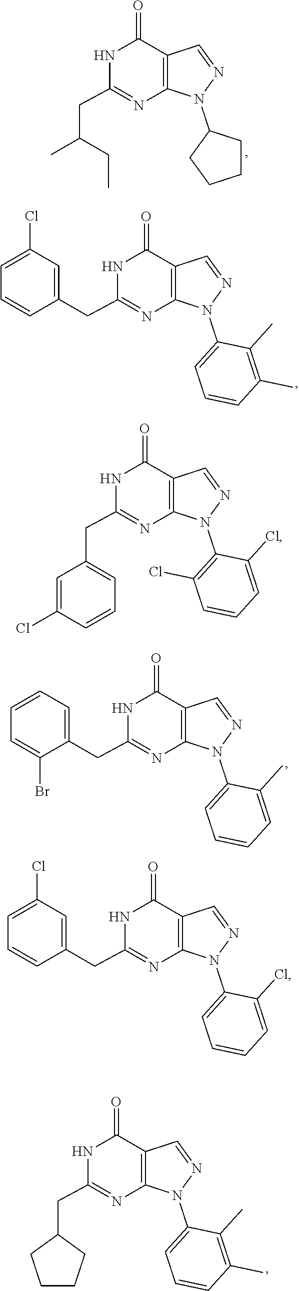 [SMILES: CCC(C)Cc1nc2c(cnn2C2CCCC2)c(=O)[nH]1 ; Cc1cccc(-n2ncc3c(=O)[nH]c(Cc4cccc(Cl)c4)nc32)c1C ; O=c1[nH]c(Cc2cccc(Cl)c2)nc2c1cnn2-c1c(Cl)cccc1Cl ; Cc1ccccc1-n1ncc2c(=O)[nH]c(Cc3ccccc3Br)nc21 ; O=c1[nH]c(Cc2cccc(Cl)c2)nc2c1cnn2-c1ccccc1Cl ; Cc1cccc(-n2ncc3c(=O)[nH]c(CC4CCCC4)nc32)c1C]

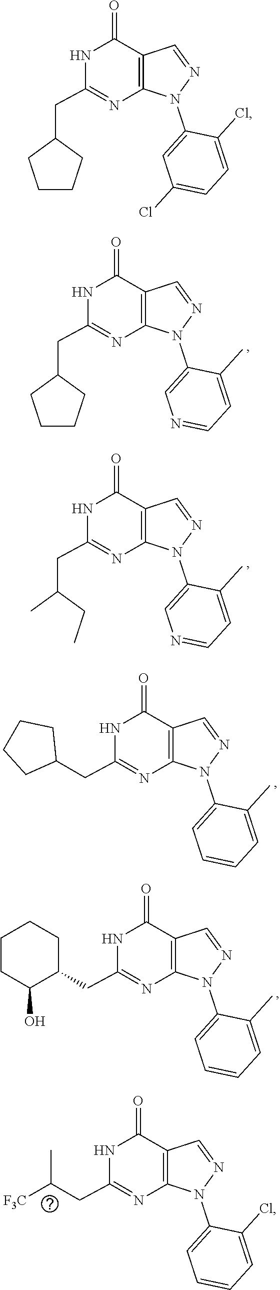

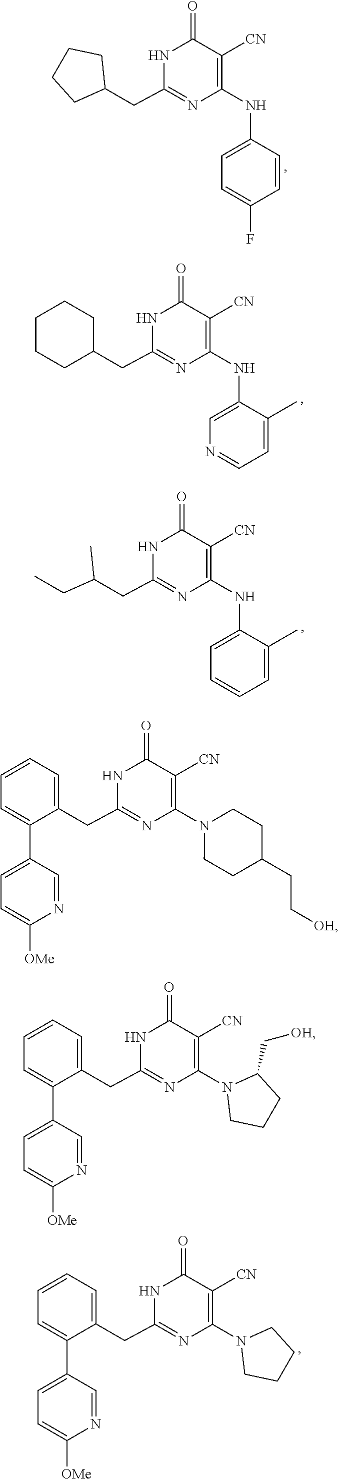

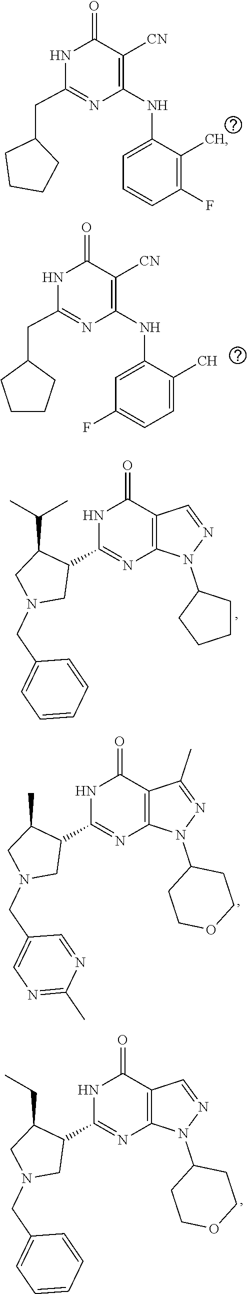

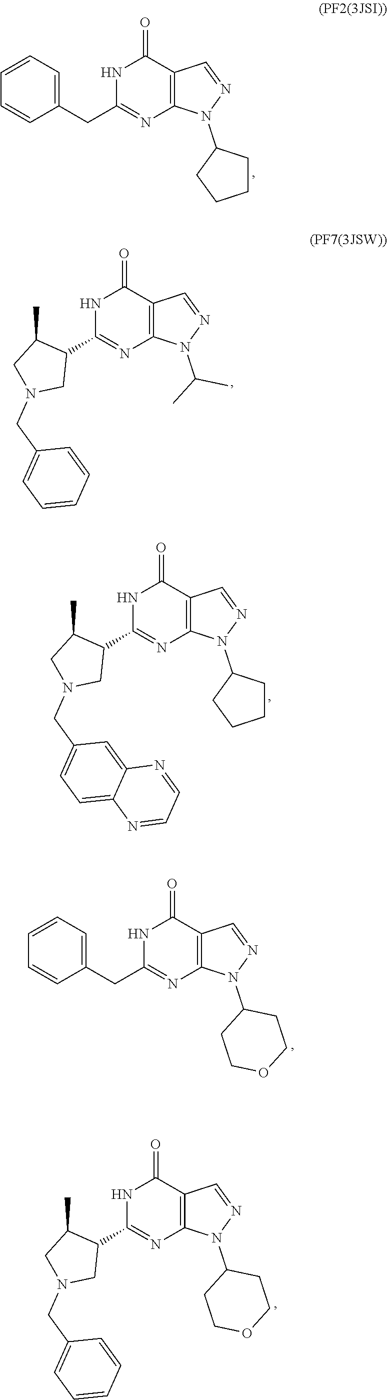

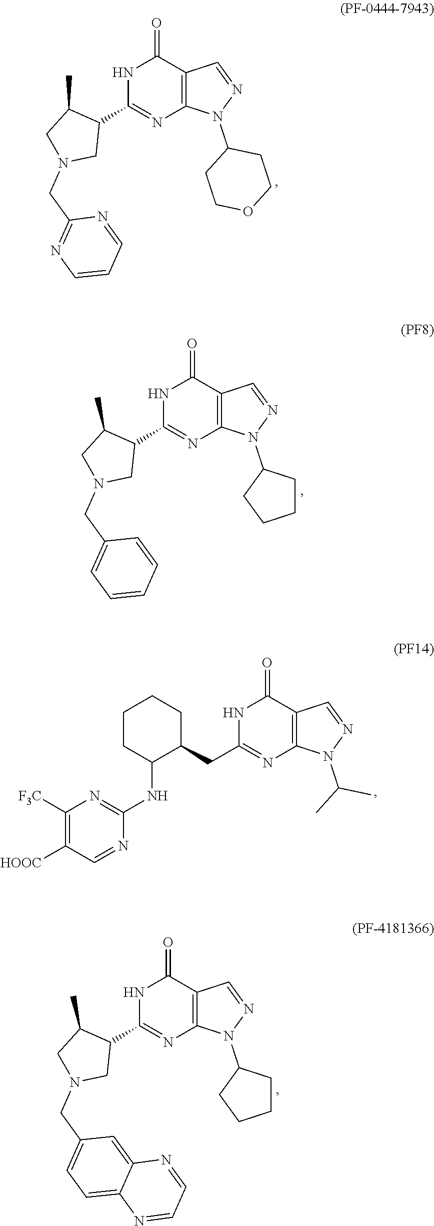

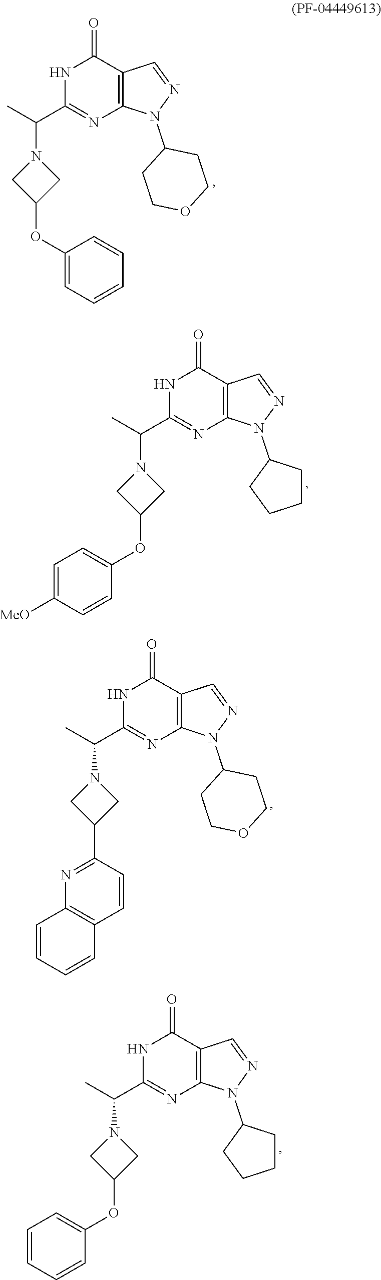 [SMILES: CC(c1nc2c(cnn2C2CCOCC2)c(=O)[nH]1)N1CC(Oc2ccccc2)C1 ; COc1ccc(OC2CN(C(C)c3nc4c(cnn4C4CCCC4)c(=O)[nH]3)C2)cc1 ; C[C@H](c1nc2c(cnn2C2CCOCC2)c(=O)[nH]1)N1CC(c2ccc3ccccc3n2)C1 ; C[C@H](c1nc2c(cnn2C2CCCC2)c(=O)[nH]1)N1CC(Oc2ccccc2)C1]

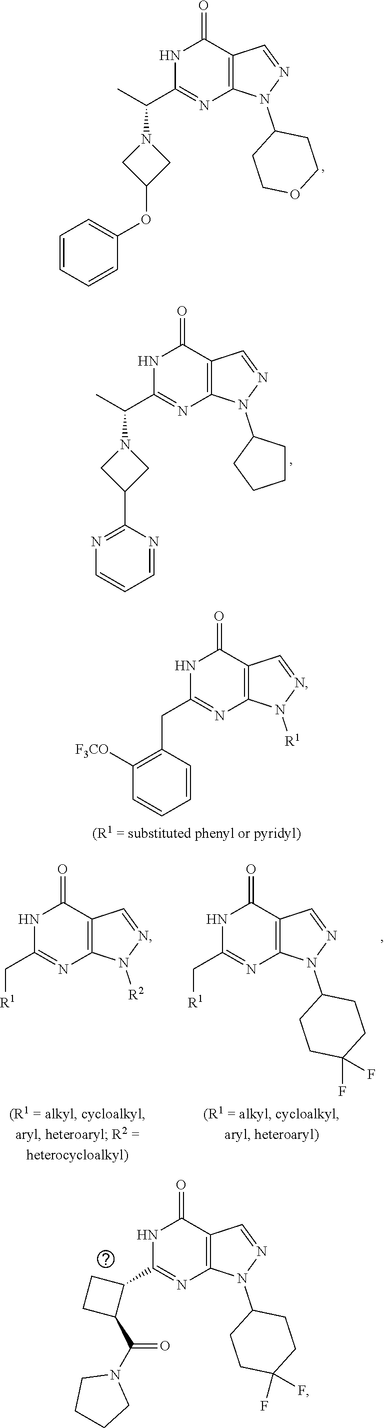

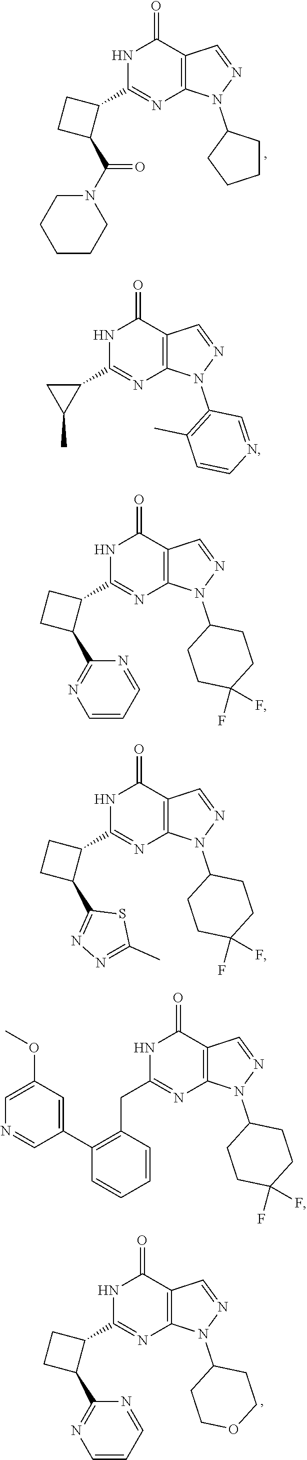

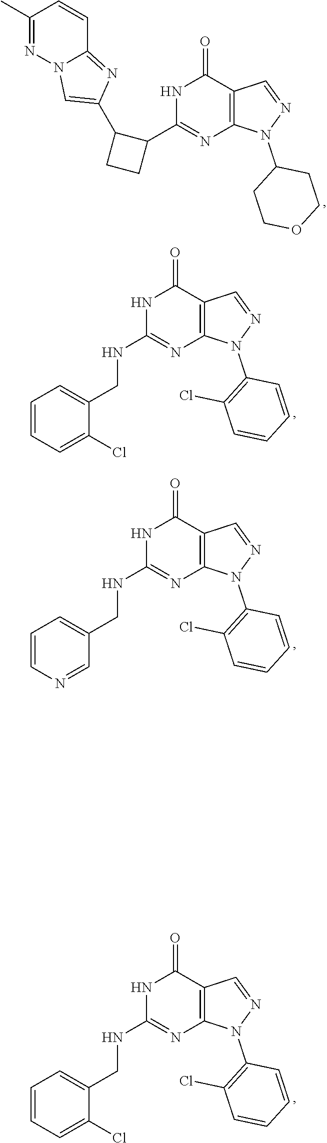 [SMILES: Cc1ccc2nc(C3CCC3c3nc4c(cnn4C4CCOCC4)c(=O)[nH]3)cn2n1 ; O=c1[nH]c(NCc2ccccc2Cl)nc2c1cnn2-c1ccccc1Cl ; O=c1[nH]c(NCc2cccnc2)nc2c1cnn2-c1ccccc1Cl ; O=c1[nH]c(NCc2ccccc2Cl)nc2c1cnn2-c1ccccc1Cl]

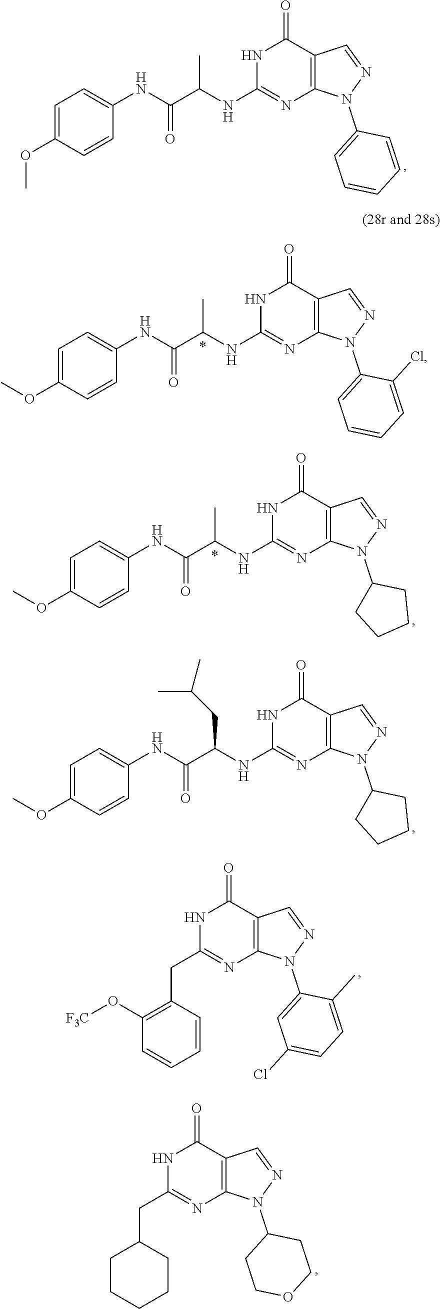 [SMILES: COc1ccc(NC(=O)C(C)Nc2nc3c(cnn3-c3ccccc3)c(=O)[nH]2)cc1 ; COc1ccc(NC(=O)C(C)Nc2nc3c(cnn3-c3ccccc3Cl)c(=O)[nH]2)cc1 ; COc1ccc(NC(=O)C(C)Nc2nc3c(cnn3C3CCCC3)c(=O)[nH]2)cc1 ; COc1ccc(NC(=O)[C@@H](CC(C)C)Nc2nc3c(cnn3C3CCCC3)c(=O)[nH]2)cc1 ; Cc1ccc(Cl)cc1-n1ncc2c(=O)[nH]c(Cc3ccccc3OC(F)(F)F)nc21 ; O=c1[nH]c(CC2CCCCC2)nc2c1cnn2C1CCOCC1]

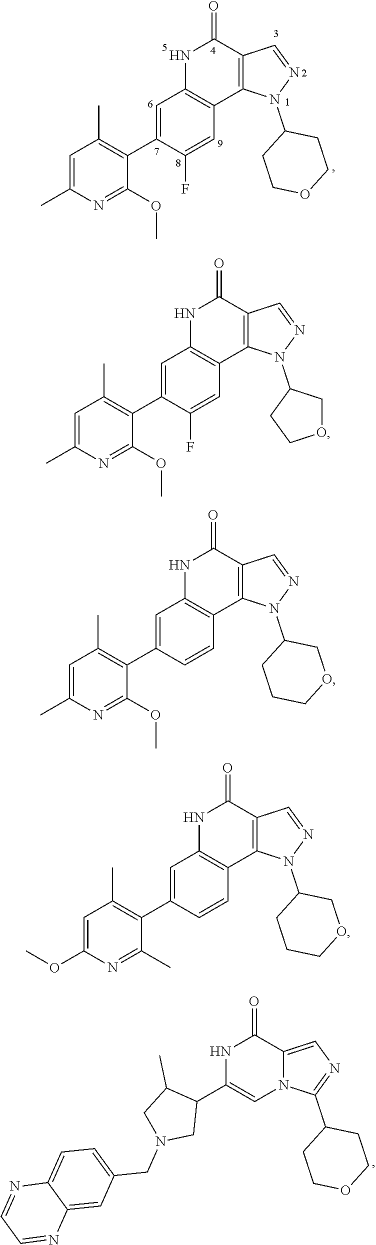 [SMILES: COc1nc(C)cc(C)c1-c1cc2[nH]c(=O)c3cnn(C4CCOCC4)c3c2cc1F ; COc1nc(C)cc(C)c1-c1cc2[nH]c(=O)c3cnn(C4CCOC4)c3c2cc1F ; COc1nc(C)cc(C)c1-c1ccc2c(c1)[nH]c(=O)c1cnn(C3CCCOC3)c12 ; COc1cc(C)c(-c2ccc3c(c2)[nH]c(=O)c2cnn(C4CCCOC4)c23)c(C)n1 ; CC1CN(Cc2ccc3nccnc3c2)CC1c1cn2c(C3CCOCC3)ncc2c(=O)[nH]1]

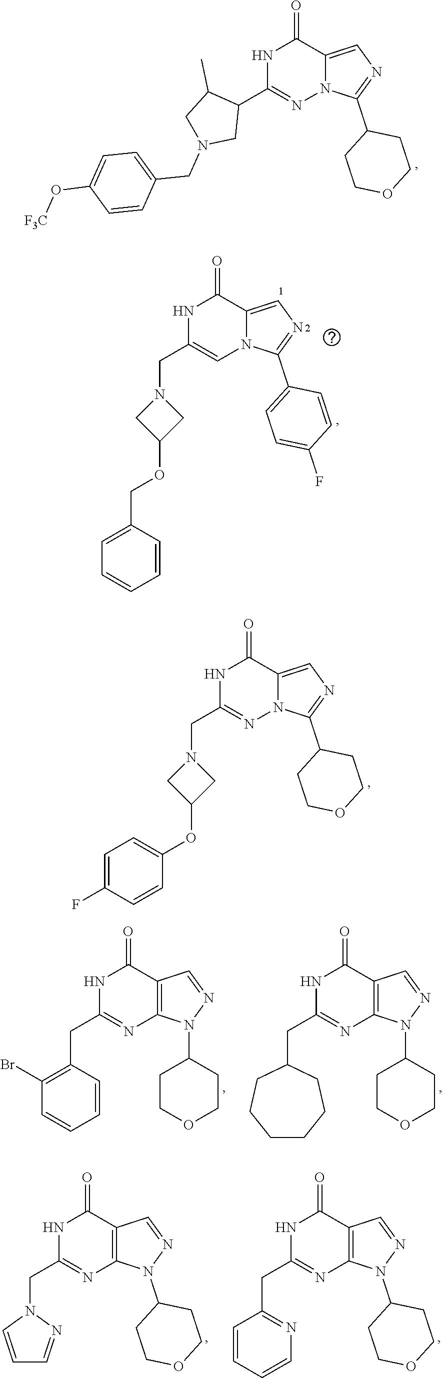

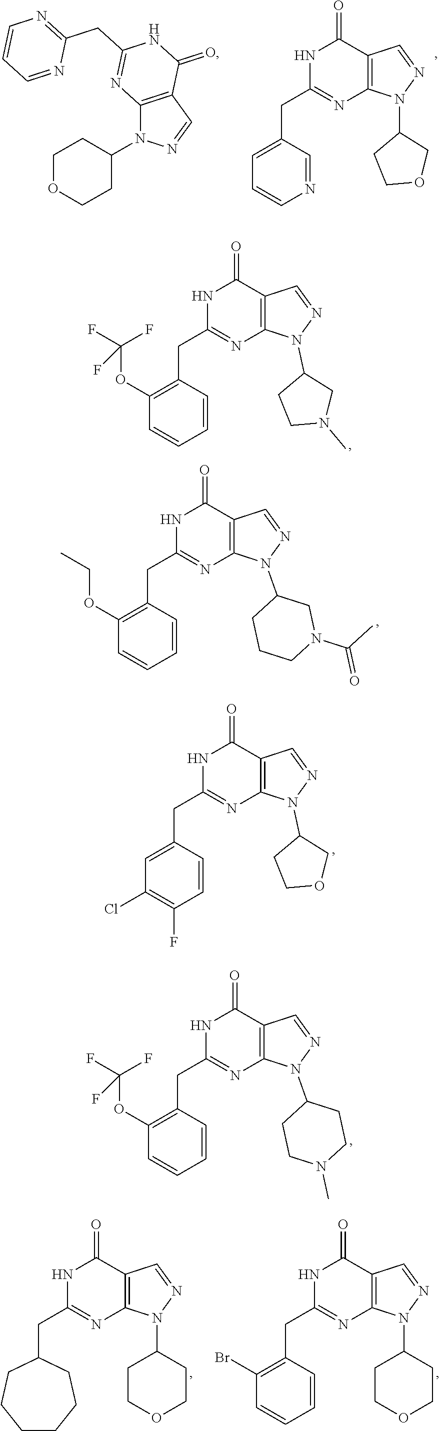

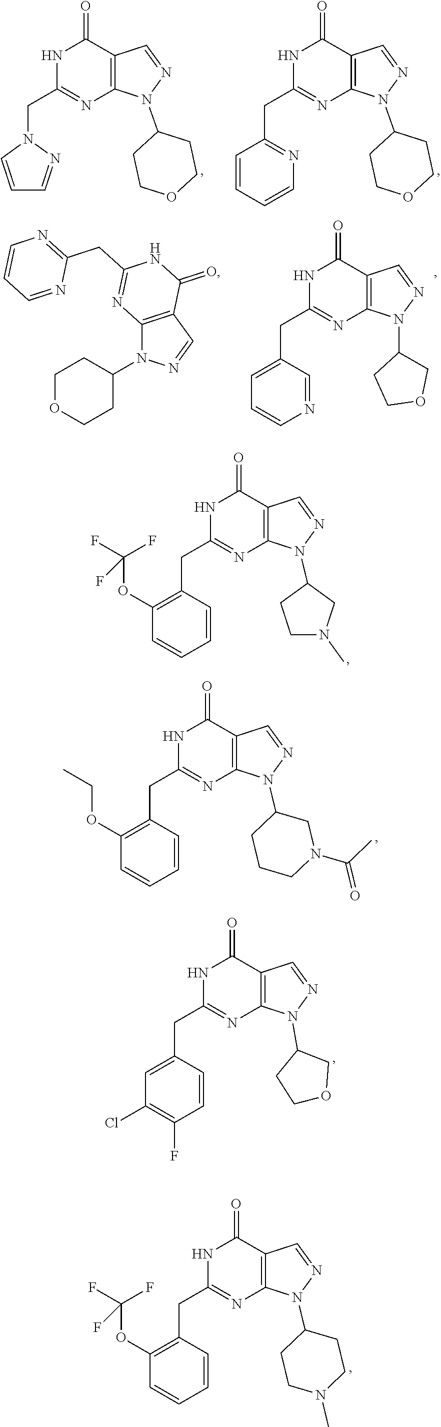

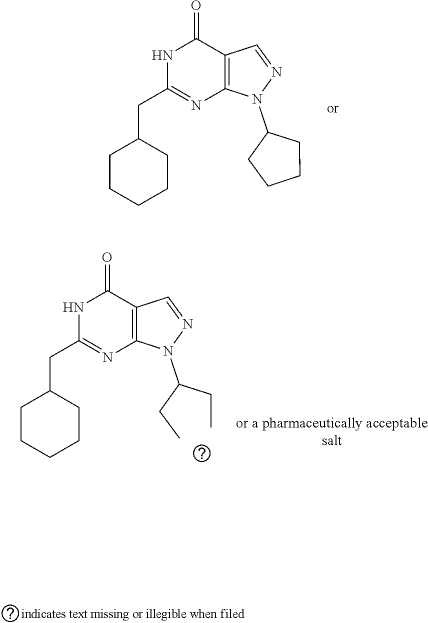 [SMILES: O=c1[nH]c(CC2CCCCC2)nc2c1cnn2C1CCCC1 ; CCC(CC)n1ncc2c(=O)[nH]c(CC3CCCCC3)nc21]

In some cases, as disclosed in WO2013051639 and U.S. Pat. No. 8,563,565, PDE9 inhibitors are represented by a compound of formula (I),

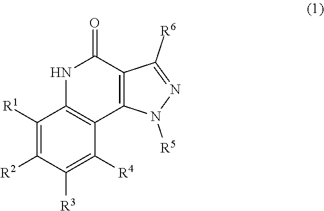

wherein
R¹ is a hydrogen atom;
R² is an aromatic ring group selected from the group consisting of a phenyl group, a pyridinyl group, and a pyrimidinyl group, where the two atoms on the aromatic ring which are adjacent to the carbon atom attached to the pyrazolo[4,3-c]quinoline ring each independently has a substituent selected from Group A1, and the other atoms on the aromatic ring independently optionally have a substituent selected from Group B1;
R³ is a hydrogen atom, or a fluorine atom;
R⁴ is a hydrogen atom;
R⁵ is an oxepanyl group, a dioxepanyl group, a tetrahydropyranyl group, or a tetrahydrofuranyl group optionally having a methoxy group;
R⁶ is a hydrogen atom;
Group A1 consists of a halogen atom, a C₁-6 alkyl group optionally having 1 to 3 halogen atoms, and a C₁-6 alkoxy group; and
Group B1 consists of a halogen atom, a cyano group, a C₁-6 alkyl group optionally having 1 to 3 halogen atoms, a C₁-6 alkoxy-C₁-6 alkyl group, a C₁-6 alkoxy group optionally having 1 to 3 halogen atoms, and a tetrahydropyranyl group,
with the proviso that when R² is a 3-pyridinyl group, the substituent at the 4-position is a halogen atom, or a C₁-6 alkyl group optionally having 1 to 3 halogen atoms.

In some embodiments, the PDE9 inhibitor is selected from the group consisting of:

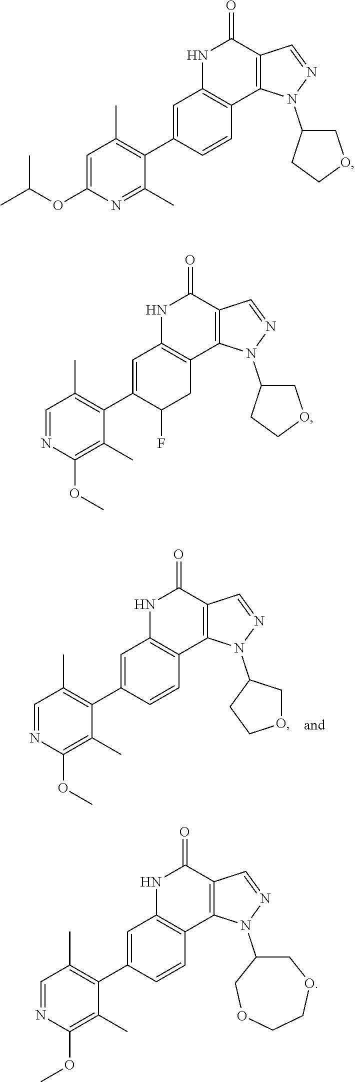 [SMILES: Cc1cc(OC(C)C)nc(C)c1-c1ccc2c(c1)[nH]c(=O)c1cnn(C3CCOC3)c12 ; COc1ncc(C)c(C2=Cc3[nH]c(=O)c4cnn(C5CCOC5)c4c3CC2F)c1C ; COc1ncc(C)c(-c2ccc3c(c2)[nH]c(=O)c2cnn(C4CCOC4)c23)c1C ; COc1ncc(C)c(-c2ccc3c(c2)[nH]c(=O)c2cnn(C4COCCOC4)c23)c1C]

In some embodiments, the PDE9 inhibitor is

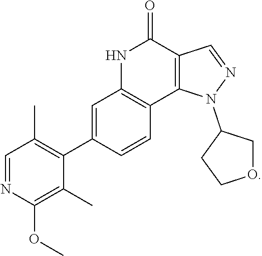 [SMILES: COc1ncc(C)c(-c2ccc3c(c2)[nH]c(=O)c2cnn(C4CCOC4)c23)c1C]

In some cases, as disclosed in CN106977518, PDE9 inhibitor is an N-substituted pyrazolo[3,4-d]pyrimidinone compound, with structure of formula (II),

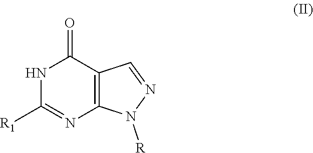

wherein, R is a cyclic or acyclic aliphatic alkyl group, a heterocyclic group, an acyl group, a hydroxyl group, or a mercapto group; R is methoxy, halogen, trifluoromethyl, ethoxy acetyl, cyano, nitro, N, N-dimethyl, chloromethyl, benzyloxy, substituted or unsubstituted amino, substituted guanidino, substituted or unsubstituted phosphoric acid, substituted or unsubstituted sulfonic acid Base, long-chain fatty alkyl, long-chain fatty amino; when R is cyclopentyl, R₁ is not substituted or unsubstituted amino.

In some cases, as disclosed in WO2003037899, PDE9 inhibitors are represented by a crystal form of compound of formula (III),

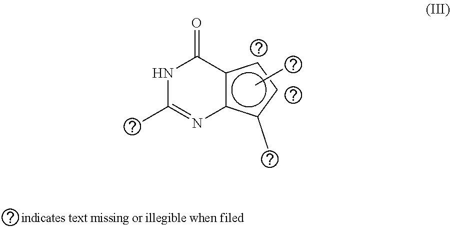

wherein
R¹ is H or C₁-6 alkyl, wherein R¹ is attached to either N¹ or N²; R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹; R³ is C₁₋₆ alkyl optionally substituted by 1 or 2 groups independently selected from: Ar²; C₃₋₇cycloalkyl optionally substituted by C₁-6 alkyl; OAr²; SAr²; NHC(O) C₁₋₆ alkyl; het²; xanthene; and naphthalene; wherein Ar¹ and Ar² are independently groups of formula,

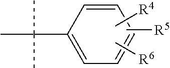

wherein R⁴, R⁵ and R⁶ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from: C₁₋₆ alkyl, C₁₋₆ alkoxy, halo and phenyl optionally substituted by 1, 2 or 3 groups independently selected from halo and C₁₋₆ alkyl; with the provisos that when a) R¹ is attached to N¹, R¹ is C₁₋₃ alkyl and R² is propyl then R³ is not methyl substituted by Ar¹, and b) R¹ is attached to N¹, R¹ is C₁₋₆ alkyl and R² is methyl then R³ is not C₁₋₄ alkyl substituted by Ar¹.

In some cases, as disclosed in WO2020187165 and US20220194934, PDE9 inhibitors are represented by a crystal form of compound of formula (IV), 6-ethyl-4-(4-methoxy-4-methylpiperidin-1-yl)-2-oxo-1,2-dihydro-1,7-two,

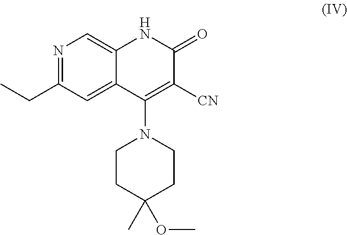 [SMILES: CCc1cc2c(N3CCC(C)(OC)CC3)c(C#N)c(=O)[nH]c2cn1]

wherein the crystalline form I of naphthalene-3-carbonitrile is characterized in that it uses Cu-Ku radiation and X-ray powder diffraction expressed in 20 angles at 7.3±0.2°, 13.6±0.2°, 14.5±0.2°, There are characteristic peaks at 18.0±0.2°, 19.1±0.2°, 22.0±0.2°, 23.4±0.2°.

In some cases, as disclosed in U.S. Pat. No. 7,964,607, PDE9 inhibitors are represented by a compound of formula (V),

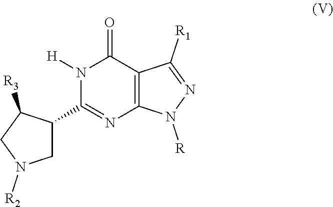

and pharmaceutically acceptable salts thereof, wherein:
R is selected from the group consisting of (C₁-C₆)alkyl, (C₂-C₆)alkenyl, (C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl;
R₁ is selected from the group consisting of hydrogen, (C₁-C₄)alkyl, (C₂-C₄)alkenyl, (C₂-C₄)alkynyl, (C₁-C₄)haloalkyl, and cyclopropyl;
R₂ is selected from the group consisting of (C₁-C₆)alkyl, (C₂-C₆)alkenyl, (C₂-C₆)alkynyl, (C₁-C₆)haloalkyl, heteroaryl selected from the group consisting of pyridinyl, pyridazinyl, pyrimidinyl, and pyrazinyl, and ER₅, wherein the heteroaryl optionally may be substituted with one to three substituents independently selected from the group consisting of (C₁-C₄)alkyl and (C₁-C₄)haloalkyl;
R₃ is selected from the group consisting of hydrogen, (C₁-C₄)alkyl, (C₁-C₄)alkenyl, (C₂-C₄)alkynyl, (C₃-C₆)cycloalkyl, and (C₁-C₄)haloalkyl;
E is selected from the group consisting of —CH₂—, —CH₂CH₂—, —CH₂CH₂CH₂—, and —C(O)—;
R₅ is selected from the group consisting of (C₃-C₈)cycloalkyl, heterocycloalkyl, aryl, aryloxy, and heteroaryl, any of which optionally may be substituted with one to three substituents, such substituents being independently selected from the group consisting of (C₁-C₄)alkyl, (C₂-C₄)alkenyl, (C₂-C₄)alkynyl, (C₁-C₄)hydroxyalkyl, (C₁-C₄)haloalkyl, (C₁-C₄)alkoxy, (C₁-C₄)haloalkoxy, (C₃-C₈)cycloalkyl, halo, cyano, phenyl, morpholinyl, (C₁-C₄)alkylamino, pyrazolyl, triazolyl, and imidazolyl.

In some embodiments, PDE9 inhibitor is selected from the group consisting of;
6-[(3,4-trans)-1-benzyl-4-methylpyrrolidin-3-yl]-1-(2-methoxyphenyl)-1,5-dihydro-4H-pyrazolo [3,4-d]pyrimidin-4-one;
6-[(3,4-trans)-1-benzyl-4-methylpyrrolidin-3-yl]-1-cyclopentyl-1,5-dihydro-4H-pyrazolo [3,4-d]pyrimidin-4-one;
6-[(3S,4S)-1-benzyl-4-methylpyrrolidin-3-yl]-1-cyclopentyl-1,5-dihydro-4H-pyrazolo [3,4-d]pyrimidin-4-one;
1-cyclopentyl-6-[(3,4-trans)-4-methyl-1-pyrimidin-2-ylpyrrolidin-3-yl]-1,5-dihydro-4H-pyrazolo [3,4-d]pyrimidin-4-one;
1-cyclopentyl-6-{(3,4-trans)-4-methyl-1-[4-(trifluoromethyl)pyrimidin-2-yl]pyrrolidin-3-yl}-1,5-dihydro-4H-pyrazolo[3,4-d]pyrimidin-4-one;
6-[(3,4-trans)-1-benzoyl-4-methylpyrrolidin-3-yl]-1-cyclopentyl-1,5-dihydro-4H-pyrazolo [3,4-d]pyrimidin-4-one;
1-cyclopentyl-6-[(3,4-trans)-4-methyl-1-(pyridin-3-ylmethyl)pyrrolidin-3-yl]-1,5-dihydro-4H-4H-pyrazolo [3,4-d]pyrimidin-4-one;
1-cyclopentyl-6-[(3,4-trans)-4-methyl-1-[3-(trifluoromethyl)benzyl]pyrrolidin-3-yl]-1,5-dihydro-4H-pyrazolo [3,4-d]pyrimidin-4-one;
1-cyclopentyl-6-[(3,4-trans)-4-methyl-1-(quinolin-2-ylmethyl)pyrrolidin-3-yl]-1,5-dihydro-4H-pyrazolo [3,4-d]pyrimidin-4-one;
1-cyclopentyl-6-[(3,4-trans)-4-methyl-1-(quinolin-4-ylmethyl)pyrrolidin-3-yl]-1,5-dihydro-4H-pyrazolo [3,4-d]pyrimidin-4-one;
1-cyclopentyl-6-[(3,4-trans)-4-methyl-1-[6-(trifluoromethyl)pyridin-3-yl]methyl pyrrolidin-3-yl]-1,5-dihydro-4H-pyrazolo[3,4-d]pyrimidin-4-one;
1-cyclopentyl-6-[(3,4-trans)-4-methyl-1-1-(quinoxalin-2-ylmethyl)pyrrolidin-3-yl]-1,5-dihydro-4H-pyrazolo[3,4-d]pyrimidin-4-one;
1-cyclopentyl-6-[(3,4-trans)-4-methyl-1-(quinoxalin-6-ylmethyl)pyrrolidin-3-yl]-1,5-dihydro-4H-pyrazolo[3,4-d]pyrimidin-4-one;
1-cyclopentyl-6-[(3,4-trans)-4-methyl-1-(pyrimidin-5-ylmethyl)pyrrolidin-3-yl]-1,5-dihydro-4H-pyrazolo[3,4-d]pyrimidin-4-one;
1-cyclopentyl-6-[(3,4-trans)-1,4-dimethylpyrrolidin-3-yl]-1,5-dihydro-4H-pyrazolo[3,4-d]pyrimidin-4-one;
1-cyclopentyl-6-[(3,4-trans)-4-methyl-1-(2,2,2-trifluoroethyl)pyrrolidin-3-yl]-1,5-dihydro-4H-pyrazolo[3,4-d]pyrimidin-4-one;
1-cyclopentyl-6-(3,4-trans)-4-methyl-1-[(2-methylpyridin-3-yl)methyl]pyrrolidin-3-yl)-1,5-dihydro-4H-pyrazolo[3,4-d]pyrimidin-4-one;
1-cyclopentyl-6-[(3,4-trans)-4-methyl-1-(quinolin-8-ylmethyl)pyrrolidin-3-yl]-1,5-dihydro-4H-pyrazolo[3,4-d]pyrimidin-4-one;
1-cyclopentyl-6-[(3,4-trans)-4-methyl-1-(quinolin-3-ylmethyl)pyrrolidin-3-yl]-1,5-dihydro-4H-pyrazolo[3,4-d]pyrimidin-4-one;
1-cyclopentyl-6-{(3,4-trans)-4-methyl-1-[(6-methylpyridin-3-yl)methyl]pyrrolidin-3-yl}-1,5-dihydro-4H-pyrazolo[3,4-d]pyrimidin-4-one;
6-[(3,4-trans)-1-benzyl-4-isopropylpyrrolidin-3-yl]-1-cyclopentyl-1,5-dihydro-4H-pyrazolo[3,4-d]pyrimidin-4-one;
6-[(3,4-trans)-1-benzyl-4-methylpyrrolidin-3-yl]-1-cyclopentyl-3-methyl-1,5-dihydro-4H-pyrazolo[3,4-d]pyrimidin-4-one;
1-cyclopentyl-6-[(3S,4S)-4-methyl-1-(quinoxalin-6-ylmethyl)pyrrolidin-3-yl]-1,5-dihydro-4H-pyrazolo[3,4-d]pyrimidin-4-one;
1-cyclopentyl-6-[(3,4-trans)-4-methyl-1-(2-phenylethyl)pyrrolidin-3-yl]-1,5-dihydro-4H-pyrazolo[3,4-d]pyrimidin-4-one;
1-cyclopentyl-6-{(3,4-trans)-1-[(6-methoxypyridin-3-yl)methyl]-4-methylpyrrolidin-3-yl}-1,5-dihydro-4H-pyrazolo[3,4-d]pyrimidin-4-one;
1-cyclopentyl-6-[(3,4-trans)-4-methyl-1-(pyridin-2-ylmethyl)pyrrolidin-3-yl]-1,5-dihydro-4H-pyrazolo[3,4-d]pyrimidin-4-one;
1-cyclopentyl-6-{(3,4-trans)-4-methyl-1-[(3-methylpyridin-2-yl)methyl]pyrrolidin-3-yl}-1,5-dihydro-4H-pyrazolo[3,4-d]pyrimidin-4-one;
6-[(3,4-trans)-1-benzyl-4-ethylpyrrolidin-3-yl]-1-cyclopentyl-1,5-dihydro-4H-pyrazolo[3,4-d]pyrimidin-4-one;
6-[(3,4-trans)-1-benzyl-4-cyclopropylpyrrolidin-3-yl]-1-cyclopentyl-1,5-dihydro-4H-pyrazolo[3,4-d]pyrimidin-4-one;
6-[(3,4-trans)-1-benzyl-4-methylpyrrolidin-3-yl]-1-isopropyl-.5-dihydro-4H-pyrazolo[3,4-d]pyrimidin-4-one;
6-[(3S,4S)-1-benzyl-4-methylpyrrolidin-3-yl]-1-isopropyl-,5-dihydro-4H-pyrazolo[3,4-d]pyrimidin-4-one;
1-isopropyl-6-[(3,4-trans)-4-methyl-1-(quinolin-2-ylmethyl)pyrrolidin-3-yl]-1,5-dihydro-4H-pyrazolo[3,4-d]pyrimidin-4-one;
1-isopropyl-6-[(3,4-trans)-4-methyl-1-(quinoxalin-6-ylmethyl)pyrrolidin-3-yl]-1,5-dihydro-4H-pyrazolo[3,4-d]pyrimidin-4-one;
1-isopropyl-6-[(3,4-trans)-4-methyl-1-(quinolin-3-ylmethyl)pyrrolidin-3-yl]-1,5-dihydro-4H-pyrazolo[3,4-d]pyrimidin-4-one;
6-[(3,4-trans)-1-benzyl-4-(trifluoromethyl)pyrrolidin-3-yl]-1-cyclopentyl-1,5-dihydro-4H-pyrazolo [3,4-d]pyrimidin-4-one;
1-isopropyl-6-[(3S,4S)-4-methyl-1-(quinoxalin-6-ylmethyl)pyrrolidin-3-yl-]-1,5-dihydro-4H-pyrazolo[3,4-d]pyrimidin-4-one;
1-isopropyl-6-[(3S,4S)-4-methyl-1-(quinolin-3-ylmethyl)pyrrolidin-3-yl]-1,5-dihydro-4H-pyrazolo[3,4-d]pyrimidin-4-one;
1-cyclopentyl-6-{(3S,4S)-4-methyl-1-[(5-methylpyrazin-2-yl)methyl]pyrrolidin-3-yl}-1,5-dihydro-4H-pyrazolo[3,4-d]pyrimidin-4-one;
6-[(3,4-trans)-1-benzyl-4-ethylpyrrolidin-3-yl]-1-isopropyl-1,5-dihydro-4H-pyrazolo[3,4-d]pyrimidin-4-one;
6-[(3S,4S)-1-benzyl-4-ethylpyrrolidin-3-yl]-1-isopropyl-1,5-dihydro-4H-pyrazolo[3,4-d]pyrimidin-4-one;
1-isopropyl-6-{(3S,4S)-4-methyl-1-[(2-methylpyrimidin-5-yl)methyl]pyrrolidin-3-yl}-1,5-dihydro-4H-pyrazolo[3,4-d]pyrimidin-4-one;
6-(1-benzylpyrrolidin-3-yl)-1-cyclopentyl-1,5-dihydro-4H-pyrazolo[3,4-d]pyrimidin-4-one;
1-isopropyl-6-(3S,4S)-1-[(6-methoxypyridin-3-yl)methyl]-4-methylpyrrolidin-3-yl-1,5-dihydro-4H-pyrazolo[3,4-d]pyrimidin-4-one;
6-[(3,4-trans)-4-ethyl-1-(quinolin-3-ylmethyl)pyrrolidin-3-yl]-1-isopropyl-1,5-dihydro-4H-pyrazolo[3,4-d]pyrimidin-4-one;
6-{(3,4-trans)-4-ethyl-1-[(6-methoxypyridin-3-yl)methyl]pyrrolidin-3-yl)}-1-isopropyl-1,5-dihydro-4H-pyrazolo[3,4-d]pyrimidin-4-one;
6-[(3,4-trans)-4-ethyl-1-(quinoxalin-6-ylmethyl)pyrrolidin-3-yl]-1-isopropyl-1,5-dihydro-4H-pyrazolo[3,4-d]pyrimidin-4-one;
1-cyclopentyl-6-{(3S,4S)-[1,3-dimethyl-1H-pyrazol-5-yl)methyl]-4-methylpyrrolidin-3-yl}-1,5-dihydro-4H-pyrazolo[3,4-d]pyrimidin-4-one;
1-cyclopentyl-6-[(3S,4S)-4-methyl-1-(4,5,6,7-tetrahydropyrazolo[1,5-a]pyridin-3-ylmethyl) pyrrolidin-3-yl]-1,5-dihydro-4H-pyrazolo[3,4-d]pyrimidin-4-one;
1-cyclopentyl-6-{(3S,4S)-4-methyl-1-[(1-methyl-1H-benzimidazol-2-yl) methyl]pyrrolidin-3-yl}-1,5-dihydro-4H-pyrazolo[3,4-d]pyrimidin-4-one;
1-isopropyl-6-{(3S,4S)-4-methyl-1-[(5-methylpyrazin-2-yl)methyl]pyrrolidin-3-yl}-1,5-dihydro-4H-pyrazolo[3,4-d]pyrimidin-4-one;
6-[(3S,4S)-1-(cinnolin-3-ylmethyl)-4-methylpyrrolidin-3-yl]-1-cyclopentyl-1,5-dihydro-4H-pyrazolo[3,4-d]pyrimidin-4-one;
1-cyclopentyl-6-[(3,4-trans)-1-(quinoxalin-6-ylmethyl)-4-(trifluoromethyl)pyrrolidin-3-yl]-1,5-dihydro-4H-pyrazolo[3,4-d]pyrimidin-4-one;
1-cyclopentyl-6-{(3S,4S)-4-methyl-1-[(2-methylpyrimidin-4-yl)methyl]pyrrolidin-3-yl}-1,5-dihydro-4H-pyrazolo[3,4-d]pyrimidin-4-one;
1-cyclopentyl-6-[(3S,4S)-1-{[2-(dimethylamino)pyrimidin-4-yl]methyl}-4-methylpyrrolidin-3-yl]-1,5-dihydro-4H-pyrazolo[3,4-d]pyrimidin-4-one;
1-cyclopentyl-6-{(3,4-trans)-4-cyclopropyl-1-[(5-methylpyrazin-2-yl)methyl]pyrrolidin-3-yl}-1,5-dihydro-4H-pyrazolo[3,4-d]pyrimidin-4-one;
1-cyclopentyl-6-[(3,4-trans)-4-cyclopropyl-1-(quinoxalin-6-ylmethyl)pyrrolidin-3-yl]-1,5-dihydro-4H-pyrazolo [3,4-d]pyrimidin-4-one;
1-cyclopentyl-6-[(3,4-trans)-4-cyclopropyl-1-methylpyrrolidin-3-yl]-1,5-dihydro-4H-pyrazolo [3,4-d]pyrimidin-4-one;
6-[(3,4-trans)-1-benzyl-4-methylpyrrolidin-3-yl]-1-ethyl-1,5-dihydro-4H-pyrazolo [3,4-d]pyrimidin-4-one;
6-{(3,4-trans)-4-ethyl-1-[(2-methylpyrimidin-5-yl)methyl]pyrrolidin-3-yl}-1-isopropyl-1,5-dihydro-4H-pyrazolo[3,4-d]pyrimidin-4-one;
6-[(3,4-trans)-1-benzyl-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-benzyl-4-methylpyrrolidin-3-yl]-1-(tetrahydro-2H-1-pyran-4-yl)-1,5-dihydro-4H-pyrazolo[3,4-d]pyrimidin-4-one;
6-[(3,4-trans)-1-benzyl-4-(2,2,2-trifluoroethyl)pyrrolidin-3-yl]-1-cyclopentyl-1,5-dihydro-4H-pyrazolo[3,4-d]pyrimidin-4-one;
6-[(3,4-trans)-1-benzyl-4-methylpyrrolidin-3-yl]-1-(4,4-difluorocyclohexyl)-1,5-dihydro-4H-pyrazolo[3,4-d]pyrimidin-4-one;
6-[(3,4-trans)-1-benzyl-4-methylpyrrolidin-3-yl]-1-(2,2,2-trifluoroethyl)-1,5-dihydro-4H-pyrazolo[3,4-d]pyrimidin-4-one;
1-isopropyl-6-[(3S,4S)-4-methyl-1-(1,5-naphthyridin-4-ylmethyl)pyrrolidin-3-yl]-1,5-dihydro-4H-pyrazolo[3,4-d]pyrimidin-4-one;
1-isopropyl-6-[(3S,4S)-4-methyl-1-(1,8-naphthyridin-4-ylmethyl)pyrrolidin-3-yl]-1,5-dihydro-4H-pyrazolo[3,4-d]pyrimidin-4-one;
1-isopropyl-6-[3S,4S)-4-methyl-1-(quinolin-4-ylmethyl)pyrrolidin-3-yl]-1,5-dihydro-4H-pyrazolo[3,4-d]pyrimidin-4-one;
1-isopropyl-6-[(3S,4S)-4-methyl-1-(pyrido[2,3-b]pyrazin-8-ylmethyl)pyrrolidin-3-yl]-1,5-dihydro-4H-pyrazolo[3,4-d]pyrimidin-4-one;
1-isopropyl-6-{(3,4-trans)-1-[(6-methoxy-1,5-naphthyridin-4-yl)methyl]-4-methylpyrrolidin-3-yl}-1,5-dihydro-4H-pyrazolo[3,4-d]pyrimidin-4-one;
6-{(3,4-trans)-1-[(8-fluoroquinolin-2-yl)methyl]-4-methylpyrrolidin-3-yl}-1-isopropyl-1,5-dihydro-4H-pyrazolo[3,4-d]pyrimidin-4-one;
1-isopropyl-6-{(v)-1-[(6-methoxyquinolin-4-yl)methyl]-4-methylpyrrolidin-3-yl}-1,5-dihydro-4H-pyrazolo[3,4-d]pyrimidin-4-one;
6-[(3,4-trans)-1-benzyl-4-methylpyrrolidin-3-yl]-1-cyclobutyl-1,5-dihydro-4H-pyrazolo[3,4-d]pyrimidin-4-one;
6-{(3S,4S)-4-methyl-1-[(2-methylpyrimidin-5-yl)methyl]pyrrolidin-3-yl}-1-(tetrahydro-2H-py ran-4-yl)-1,5-dihydro-4-t-pyrazolo[3,4-d]pyrimidin-4-one;
6-{(3,4-trans)-4-ethyl-1-[(2-methylpyrimidin-5-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-4-methyl-1-[(5-methylpyrazin-2-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-1-[(6-methoxypyridin-3-yl)methyl]-4-methylpyrrolidin-3-yl}-1-(tetrahydro-2H-pyran-4-yl)-1,5-dihydro-4H-pyrazolo[3,4-d]pyrimidin-4-one;
6-[(3S,4S)-4-methyl-1-(quinolin-3-ylmethyl)pyrrolidin-3-yl]-1-(tetrahydro-2H-pyran-4-yl)-1,5-dihydro-4H-pyrazolo[3,4-d]pyrimidin-4-one;
6-{(3S,4S)-4-methyl-1-[(2-methylpyrimidin-4-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-4-methyl-1-[(6-methylpyridin-3-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-4-methyl-1-}([6-(trifluoromethyl)pyridin-3-yl]methyl}pyrrolidin-3-yl]-1-(tetrahydro-2H-pyran-4-yl)-1,5-dihydro-4H-pyrazolo[3,4-d]pyrimidin-4-one;
6-{(3S,4S)-4-methyl-1-1[(1-methyl-1H-imidazo[4,5-c]pyridin-2-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-1-[(1,3-dimethyl-1H-pyrazol-5-yl)methyl]-4-methylpyrrolidin-3-yl}-1-(tetrahydro-2H-pyran-4-yl)-1,5-dihydro-4H-pyrazolo[3,4-d]pyrimidin-4-one;
1-cyclobutyl-6-{(3,4-trans)-4-methyl-1-[(2-methylpyrimidin-5-yl)methyl]pyrrolidin-3-yl}-1,5-dihydro-4H-pyrazolo[3,4-d]pyrimidin-4-one;
6-[(3S,4S)-1-(2,1,3-benzothiadiazol-5-ylmethyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-4-methyl-1-(quinoxalin-2-ylmethyl)pyrrolidin-3-yl]-1-(tetrahydro-2H-pyran-4-yl)-1,5-dihydro-4H-pyrazolo[3,4-d]pyrimidin-4-one;
6-[(3S,4S)-4-methyl-1-(quinolin-4-ylmethyl)pyrrolidin-3-yl]-1-tetrahydro-2H-pyran-4-yl)-1,5-dihydro-4H-pyrazolo[3,4-d]pyrimidin-4-one;
6-[(3S,4S)-4-methyl-1-(pyridin-2-ylmethyl)pyrrolidin-3-yl]-1-(tetrahydro-2H-pyran-4-yl)-1,5-dihydro-4H-pyrazolo[3,4-d]pyrimidin-4-one;
6-[(3S,4S)-1-benzyl-4-methylpyrrolidin-3-yl]-3-methyl-1-(tetrahydro-2H-pyran-4-yl)-1,5-dihydro-4H-pyrazolo[3,4-d]pyrimidin-4-one;
6-[(3S,4S)-1-(3-fluorobenzyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-(35-difluorobenzyl)-4-methylpyrrolidin-3-yl]-1-(tetrahydro-2H-pyran-4-yl)-1,5-dihydro-4H-pyrazolo[3,4-d]pyrimidin-4-one;
6-{(3S,4S)-4-methyl-1-[4-(trifluoromethyl)benzyl]pyrrolidin-3-yl}-1-(tetrahydro-2H-pyran-4-yl)-1,5-dihydro-4H-pyrazolo[3,4-d]pyrimidin-4-one;
6-[(3,4-trans)-1-benzyl-4-ethylpyrrolidin-3-yl]-1-(tetrahydro-2H-pyran-4-yl)-1,5-dihydro-4H-pyrazolo[3,4-d]pyrimidin-4-one;
6-[(3S,4S)-1-benzyl-4-ethylpyrrolidin-3-yl]-1-(tetrahydro-2H-pyran-4-yl)-1,5-dihydro-4H-pyrazolo[3,4-d]pyrimidin-4-one;
3-methyl-6-[(3S,4S)-4-methyl-1-(pyridin-3-ylmethyl)pyrrolidin-3-yl]-1-(tetrahydro-2H-pyran-4-yl)-1,5-dihydro-4H-pyrazolo[3,4-d]pyrimidin-4-one;
3-methyl-6-{(3S,4S)-4-methyl-1-[(2-methylpyrimidin-5-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-1-[(6-methoxypyridin-3-yl)methyl]-4-methylpyrrolidin-3-yl}-3-methyl-1-(tetrahydro-2H-pyran-4-yl)-1,5-dihydro-4H-pyrazolo[3,4-d]pyrimidin-4-one;
6-{(3S,4S)-4-methyl-1-[(6-methylpyridin-2-yl)methyl]pyrrolidin-3-yl}-1-(tetrahydro-2H-pyran-4-yl)-1,5-dihydro-4H-pyrazolo[3,4-d]pyrimidin-4-one;
6-[(3S,4S)-1-(4-fluorobenzyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-(2-fluorobenzyl)-4-methylpyrrolidin-3-yl]-1-(tetrahydro-2H-pyran-4-yl)-1,5-dihydro-4H-pyrazolo[3,4-d]pyrimidin-4-one;
6-{(3S,4S)-4-methyl-1-[2-(trifluoromethyl)benzyl]pyrrolidin-3-yl}-1-(tetrahydro-2H-pyran-4-yl)-1,5-dihydro-4H-pyrazolo[3,4-d]pyrimidin-4-one;
6-[(3S,4S)-1-(2,4-difluorobenzyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-(4-methoxybenzyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-benzyl-4-methylpyrrolidin-3-yl]-1-(tetrahydro-2H-thiopyran-4-yl)-1,5-dihydro-4H-pyrazolo[3,4-d]pyrimidin-4-one;
6-[(3S,4S)-1-(2-methoxybenzyl)-4-methylpyrrolidin-3-yl]-1-(tetrahydro-2H-pyran-4-yl)-1,5-dihydro-4H-pyrazolo[3,4-d]pyrimidin-4-one;
6-[(3S,4S)-1-(3-methoxybenzyl)-4-methylpyrrolidin-3-yl]-1-(tetrahydro-2H-pyran-4-yl)-1,5-dihydro-4H-pyrazolo[3,4-d]pyrimidin-4-one;
6-{(3S,4S)-4-methyl-1-[3-(trifluoromethyl)benzyl]pyrrolidin-3-yl}-1-(tetrahydro-2H-pyran-4-yl)-1,5-dihydro-4H-pyrazolo[3,4-d]pyrimidin-4-one;
6-[(3S,4S)-1-(26-difluorobenzyl)-4-methylpyrrolidin-3-yl]-1-(tetrahydro-2H-pyran-4-yl)-1,5-dihydro-4H-pyrazolo[3,4-d]pyrimidin-4-one;
6-{(3S,4S)-4-ethyl-[(5-methylpyrazin-2-yl)methyl]pyrrolidin-3-yl}-1-(tetrahydro-2H-pyran-4-yl)-1,5-dihydro-4H-pyrazolo[3,4-d]pyrimidin-4-one;
6-{(3S,4S)-4-ethyl-1-[(6-methoxypyridin-3-yl)methyl]pyrrolidin-3-yl}-1-(tetrahydro-2H-pyran-4-yl)-1,5-dihydro-4H-pyrazolo[3,4-d]pyrimidin-4-one;
6-[(3S,4S)-4-ethyl-1-(pyridin-2-ylmethyl)pyrrolidin-3-yl]-1-(tetrahydro-2H-pyran-4-yl)-1,5-dihydro-4H-pyrazolo[3,4-d]pyrimidin-4-one;
6-[(3S,4S)-4-ethyl-1-(quinoxalin-2-ylcarbonyl)pyrrolidin-3-yl]-1-(tetrahydro-2H-pyran-4-yl)-1,5-dihydro-4H-pyrazolo[3,4-d]pyrimidin-4-one;
6-[(3S,4S)-4-methyl-1-(pyrimidin-2-ylmethyl)pyrrolidin-3-yl]-1-(tetrahydro-2H-pyran-4-yl)-1,5-dihydro-4H-pyrazolo[3,4-d]pyrimidin-4-one;
2-({(3S,4S)-3-ethyl-4-[4-oxo-1-(tetrahydro-2H-pyran-4-yl)-45-dihydro-1H-pyrazolo[3,4-d]pyrimidin-6-yl]pyrrolidin-1-yl}methyl)benzonitrile;
3-({(3S,4S)-3-ethyl-4-[4-oxo-1-(tetrahydro-2H-pyran-4-yl)-45-dihydro-1H-pyrazolo[3,4-d]pyrimidin-6-yl]pyrrolidin-1-yl}methyl)benzonitrile;
4-({(3S,4S)-3-ethyl-4-[4-oxo-1-(tetrahydro-2H-pyran-4-yl)-45-dihydro-1H-pyrazolo[3,4-d]pyrimidin-6-yl]pyrrolidin-1-yl}methyl)benzonitrile;
1-cyclopentyl-6-(3,4-trans)-4-methyl-1-[3-(1H-pyrazol-1-yl)benzyl]pyrrolidin-3-yl}-1,5-dihydro-4H-pyrazolo[3,4-d]pyrimidin-4-one;
1-cyclopentyl-6-{(3,4-trans)-4-methyl-1-[(2-methylpyridin-4-yl)methyl]pyrrolidin-3-yl}-1,5-dihydro-4H-pyrazolo[3,4-d]pyrimidin-4-one;
6-[(3,4-trans)-1-(2-chloro-6-fluorobenzyl)-4-methylpyrrolidin-3-yl]-1-cyclopentyl-1,5-dihydro-4H-pyrazolo[3,4-d]pyrimidin-4-one;
1-cyclopentyl-6-[(3,4-trans)-1-(2,3-dimethylbenzyl)-4-methylpyrrolidin-3-yl]-1,5-dihydro-4H-pyrazolo[3,4-d]pyrimidin-4-one;
1-cyclopentyl-6-{(3,4-trans)-1-[2-(difluoromethoxy)benzyl]-4-methylpyrrolidin-3-yl}-1,5-dihydro-4H-pyrazolo[3,4-]pyrimidin-4-one;
1-cyclopentyl-6-{(3,4-trans)-1-[(2-ethoxypyridin-3-yl)methyl]-4-methylpyrrolidin-3-yl}-1,5-dihydro-4H-pyrazolo[3,4-d]pyrimidin-4-one;
1-cyclopentyl-6-[(3,4-trans)-4-methyl-1-(4,5,67-tetrahydropyrazolo[1,5-a]pyridin-3-ylmethyl)pyrrolidin-3-yl]-1,5-dihydro-4H-pyrazolo[3,4-d]pyrimidin-4-one;
1-cyclopentyl-6-[(3,4-trans)-1-(2,3-dihydro-1,4-benzodioxin-6-ylmethyl)-4-methylpyrrolidin-3-yl]-1,5-dihydro-4H-pyrazolo[3,4-d]pyrimidin-4-one;
1-cyclopentyl-6-{(3,4-trans)-1-[4-(1H-imidazol-1-yl)benzyl]-4-methylpyrrolidin-3-yl}-1,5-dihydro-4H-pyrazolo[3,4-d]pyrimidin-4-one;
1-cyclopentyl-6-[(3,4-trans)-1-(2,5-dichlorobenzyl)-4-methylpyrrolidin-3-yl]-1,5-dihydro-4H-pyrazolo[3,4-d]pyrimidin-4-one;
1-cyclopentyl-6-[(3,4-trans)-1-(4-methoxy-3-methylbenzyl)-4-methylpyrrolidin-3-yl]-1,5-dihydro-4H-pyrazolo[3,4-d]pyrimidin-4-one;
1-cyclopentyl-6-[(3,4-trans)-1-(2,3-dihydro-1-benzofuran-7-ylmethyl)-4-methylpyrrolidin-3-yl]-1,5-dihydro-4H-pyrazolo[3,4-d]pyrimidin-4-one;
1-cyclopentyl-6-[(3,4-trans)-1-(2,3-difluorobenzyl)-4-methylpyrrolidin-3-yl]-1,5-dihydro-4H-pyrazolo[3,4-d]pyrimidin-4-one;
1-cyclopentyl-6-[(3,4-trans)-1-(5-fluoro-2-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(2-fluoro-4-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(3-fluoro-4-methylbenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(2-methyl-1,3-thiazol-5-yl)methyl]pyrrolidin-3-yl}-1,5-dihydro-4H-pyrazolo[3,4-d]pyrimidin-4-one;
1-cyclopentyl-6-{(3,4-trans)-1-[(4-isopropyl-1,3-thiazol-2-yl)methyl]-4-methylpyrrolidin-3-yl}-1,5-dihydro-4H-pyrazolo[3,4-d]pyrimidin-4-one;
1-cyclopentyl-6-{(3,4-trans)-1-[(1,3-dimethyl-1H-pyrazol-5-yl)methyl]-4-methylpyrrolidin-3-yl}-1,5-dihydro-4H-pyrazolo[3,4-d]pyrimidin-4-one;
1-cyclopentyl-6-[(3,4-trans)-1-(2,3-difluoro-4-methylbenzyl)-4-methylpyrrolidin-3-yl]-1,5-dihydro-4H-pyrazolo[3,4-d]pyrimidin-4-one;
1-cyclopentyl-6[-(3,4-trans)-4-methyl-1-{[6-(1H-pyrazol-1-yl)pyridin-2-yl]methyl}pyrrolidin-3-yl]-1,5-dihydro-4H-pyrazolo[3,4-d]pyrimidin-4-one;
1-cyclopentyl-6-[(3,4-trans)-4-methyl-1-(4-methylbenzyl)pyrrolidin-3-yl]-1,5-dihydro-4H-pyrazolo[3,4-d]pyrimidin-4-one;
1-cyclopentyl-6-[(3,4-trans)-4-methyl-1-(2-naphthylmethyl)pyrrolidin-3-yl]-1,5-dihydro-4H-pyrazolo[3,4-d]pyrimidin-4-one;
1-cyclopentyl-6-{(3,4-trans)-1-[(2-methoxypyridin-3-yl)methyl]-4-methylpyrrolidin-3-yl}-1,5-dihydro-4H-pyrazolo[3,4-d]pyrimidin-4-one;
1-cyclopentyl-6-[(3,4-trans)-1-(2-ethoxybenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[4-(1H-1,2,4-triazol-1-yl)benzyl]pyrrolidin-3-yl}-1,5-dihydro-4H-pyrazolo[3,4-d]pyrimidin-4-one;
1-cyclopentyl-6-[(3,4-trans)-1-(3-methoxy-4-methylbenzyl)-4-methylpyrrolidin-3-yl]-1,5-dihydro-4H-pyrazolo[3,4-d]pyrimidin-4-one;
1-cyclopentyl-6-[(3,4-trans)-4-methyl-1-(1-naphthylmethyl)pyrrolidin-3-yl]-1,5-dihydro-4H-pyrazolo[3,4-d]pyrimidin-4-one;
1-cyclopentyl-6-[(3,4-trans)-1-(3-fluoro-4-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(2,5-dimethoxybenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(5-methylisoxazol-3-yl)methyl]pyrrolidin-3-yl}-1,5-dihydro-4H-pyrazolo[3,4-d]pyrimidin-4-one;
1-cyclopentyl-6-[(3,4-trans)-1-(2-fluoro-6-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(2,4-difluorobenzyl)-4-methylpyrrolidin-3-yl]-1,5-dihydro-4H-pyrazolo[3,4-d]pyrimidin-4-one;
1-cyclopentyl-6-[(3,4-trans)-1-(4-fluoro-3-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(2,3-dihydro-1,4-benzodioxin-5-ylmethyl)-4-methylpyrrolidin-3-yl]-1,5-dihydro-4H-pyrazolo[3,4-d]pyrimidin-4-one;
6-[(3,4-trans)-1-(2-chloro-4-fluorobenzyl)-4-methylpyrrolidin-3-yl]-1-cyclopentyl-1,5-dihydro-4H-pyrazolo[3,4-d]pyrimidin-4-one;
1-cyclopentyl-6-[(3,4-trans)-1-(2,4-dimethylbenzyl)-4-methylpyrrolidin-3-yl]-1,5-dihydro-4H-pyrazolo[3,4-d]pyrimidin-4-one;
1-cyclopentyl-6-[(3,4-trans)-1-(3,5-dimethoxybenzyl)-4-methylpyrrolidin-3-yl]-1,5-dihydro-4H-pyrazolo[3,4-d]pyrimidin-4-one;
1-cyclopentyl-6-[(3,4-trans)-1-(3-ethoxybenzyl)-4-methylpyrrolidin-3-yl]-1,5-dihydro-4H-pyrazolo[3,4-d]pyrimidin-4-one;
6-[(3,4-trans)-1-(4-chloro-2-fluorobenzyl)-4-methylpyrrolidin-3-yl]-1-cyclopentyl-1,5-dihydro-4H-pyrazolo[3,4-d]pyrimidin-4-one;
3-{[(3,4-trans)-3-(1-cyclopentyl-4-oxo-4,5-dihydro-1H-pyrazolo[3,4-d]pyrimidin-6-yl)-4-methylpyrrolidin-1-yl]methyl}benzonitrile;
1-cyclopentyl-6-[(3,4-trans)-1-(2,5-difluorobenzyl)-4-methylpyrrolidin-3-yl]-1,5-dihydro-4H-pyrazolo[3,4-d]pyrimidin-4-one;
2-{[(3,4-trans)-3-(1-cyclopentyl-4-oxo-4,5-dihydro-1H-pyrazolo[3,4-d]pyrimidin-6-yl)-4-methylpyrrolidin-1-yl]methyl}benzonitrile;
6-[(3,4-trans)-1-(3-chloro-4-fluorobenzyl)-4-methylpyrrolidin-3-yl]-1-cyclopentyl-1,5-dihydro-4H-pyrazolo[3,4-d]pyrimidin-4-one;
1-cyclopentyl-6-{(3,4-trans)-1-[4-(difluoromethoxy)benzyl]-4-methylpyrrolidin-3-yl}-1,5-dihydro-4H-pyrazolo[3,4-d]pyrimidin-4-one;
1-cyclopentyl-6-[(3,4-trans)-4-methyl-1-(3-methylbenzyl)pyrrolidin-3-yl]-1,5-dihydro-4H-pyrazolo[3,4-d]pyrimidin-4-one;
1-cyclopentyl-6-[(3,4-trans)-1-(3,4-difluorobenzyl)-4-methylpyrrolidin-3-yl]-1,5-dihydro-4H-pyrazolo[3,4-d]pyrimidin-4-one;
1-cyclopentyl-6-[(3,4-trans)-1-(2,5-dimethylbenzyl)-4-methylpyrrolidin-3-yl]-1,5-dihydro-4H-pyrazolo[3,4-d]pyrimidin-4-one;
6-[(3,4-trans)-1-(3-chloro-2-fluorobenzyl)-4-methylpyrrolidin-3-yl]-1-cyclopentyl-1,5-dihydro-4H-pyrazolo[3,4-d]pyrimidin-4-one;
1-cyclopentyl-6-[(3,4-trans)-1-(2,3-dichlorobenzyl)-4-methylpyrrolidin-3-yl]-1,5-dihydro-4H-pyrazolo[3,4-d]pyrimidin-4-one;
1-cyclopentyl-6-[(3,4-trans)-4-methyl-1-(1,3-thiazol-2-ylmethyl)pyrrolidin-3-yl]-1,5-dihydro-4H-pyrazolo[3,4-d]pyrimidin-4-one;
1-cyclopentyl-6-[(3,4-trans)-1-(3-fluoro-2-methylbenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(2-methylpyrimidin-5-yl)methyl]pyrrolidin-3-yl}-1,5-dihydro-4H-pyrazolo[3,4-d]pyrimidin-4-one;
1-cyclopentyl-6-{(3,4-trans)-1-[(2-ethylpyrimidin-5-yl)methyl]-4-methylpyrrolidin-3-yl}-1,5-dihydro-4H-pyrazolo[3,4-d]pyrimidin-4-one;
1-cyclopentyl-6-[(3,4-trans)-1-(4-isopropylbenzyl)-4-methylpyrrolidin-3-yl]-1,5-dihydro-4H-pyrazolo[3,4-d]pyrimidin-4-one;
1-cyclopentyl-6-{(3,4-trans-1-[(1-ethyl-1H-pyrazol-4-yl)methyl]-4-methylpyrrolidin-3-yl}-1,5-dihydro-4H-pyrazolo[3,4-d]pyrimidin-4-one;
1-cyclopentyl-6-{(3,4-trans)-1-[(4-methoxypyridin-3-yl)methyl]-4-methylpyrrolidin-3-yl}-1,5-dihydro-4H-pyrazolo[3,4-d]pyrimidin-4-one;
1-cyclopentyl-6-[(3,4-trans)-1-(isoxazol-5-ylmethyl)-4-methylpyrrolidin-3-yl]-1,5-dihydro-4H-pyrazolo[3,4-d]pyrimidin-4-one;
1-cyclopentyl-6-[(3,4-trans)-1-(4-ethoxybenzyl)-4-methylpyrrolidin-3-yl]-1,5-dihydro-4H-pyrazolo[3,4-d]pyrimidin-4-one;
1-cyclopentyl-6-[(3,4-trans)-1-{[6-(1-hydroxy-1-methylethyl)pyridin-3-yl]methyl}-4-methylpyrrolidin-3-yl]-1,5-dihydro-4H-pyrazolo[3,4-d]pyrimidin-4-one;
1-cyclopentyl-6-{(3,4-trans)-1-[(2,2-dimethyl-2,3-dihydro-1-benzofuran-5-yl)methyl]-4-methylpyrrolidin-3-yl}-1,5-dihydro-4H-pyrazolo[3,4-d]pyrimidin-4-one;
1-cyclopentyl-6-[(3,4-trans)-1-(3,4-dimethoxybenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(5-methylpyrazin-2-yl)methyl]pyrrolidin-3-yl}-1,5-dihydro-4H-pyrazolo[3,4-d]pyrimidin-4-one;
1-cyclopentyl-6-[(3,4-trans)-1-(imidazo[1,2-a]pyridin-2-ylmethyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(2-phenyl-1,3-oxazol-4-yl)methyl]pyrrolidin-3-yl}-1,5-dihydro-4H-pyrazolo[3,4-d]pyrimidin-4-one;
1-cyclopentyl-6-[(3,4-trans)-4-methyl-1-(2-methylbenzyl)pyrrolidin-3-yl]-1,5-dihydro-4H-pyrazolo[3,4-d]pyrimidin-4-one;
1-cyclopentyl-6-[(3,4-trans)-1-(2-isopropoxybenzyl)-4-methylpyrrolidin-3-yl]-1,5-dihydro-4H-pyrazolo[3,4-d]pyrimidin-4-one;
6-[(3,4-trans)-1-(cinnolin-3-ylmethyl)-4-methylpyrrolidin-3-yl]-1-cyclopentyl-1,5-dihydro-4H-pyrazolo[3,4-d]pyrimidin-4-one;
1-cyclopentyl-6-{(3,4-trans)-1-[3-(difluoromethoxy)benzyl]-4-methylpyrrolidin-3-yl}-1,5-dihydro-4H-pyrazolo[3,4-d]pyrimidin-4-one;
1-cyclopentyl-6-[(3,4-trans)-1-(4-fluoro-3-methylbenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[4-(1H-1-pyrazol-1-yl)benzyl]pyrrolidin-3-yl}-1,5-dihydro-4H-pyrazolo[3,4-d]pyrimidin-4-one;
1-cyclopentyl-6-{(3,4-trans)-1-[(2,7-dimethylimidazo[1,2-a]pyridin-3-yl)methyl]-4-methylpyrrolidin-3-yl}-1,5-dihydro-4H-pyrazolo[3,4-d]pyrimidin-4-one;
1-cyclopentyl-6-[(3,4-trans)-1-(3,5-dichlorobenzyl)-4-methylpyrrolidin-3-yl]-1,5-dihydro-4H-pyrazolo[3,4-d]pyrimidin-4-one;
1-cyclopentyl-6-[(3,4-trans)-1-(4-isopropoxy benzyl)-4-methylpyrrolidin-3-yl]-1,5-dihydro-4H-pyrazolo[3,4-d]pyrimidin-4-one;
1-cyclopentyl-6-[(3,4-trans)-1-{[2-(1-hydroxy-1-methylethyl)pyridin-4-yl]methyl}-4-methylpyrrolidin-3-yl]-1,5-dihydro-4H-pyrazolo[3,4-d]pyrimidin-4-one;
1-cyclopentyl-6-[(3,4-trans)-4-methyl-1-(4,5,6,7-tetrahydro-1,3-benzothiazol-2-ylmethyl)pyrrolidin-3-yl]-1,5-dihydro-4H-pyrazolo[3,4-d]pyrimidin-4-one;
1-cyclopentyl-6-[(3,4-trans)-1-(mesitylmethyl)-4-methylpyrrolidin-3-yl]-1,5-dihydro-4H-pyrazolo[3,4-d]pyrimidin-4-one;
1-cyclopentyl-6-[(3,4-trans)-1-(2,6-dichlorobenzyl)-4-methylpyrrolidin-3-yl]-1,5-dihydro-4H-pyrazolo[3,4-d]pyrimidin-4-one;
4-{[(3,4-trans)-3-(1-cyclopentyl-4-oxo-4,5-dihydro-1H-pyrazolo[3,4-d]pyrimidin-6-yl)-4-methylpyrrolidin-1-yl]methyl}benzonitrile;
1-cyclopentyl-6-[(3,4-trans)-1-(2-fluoro-5-methoxybenzyl)-4-methylpyrrolidin-3-yl]-1,5-dihydro-4H-pyrazolo[3,4-d]pyrimidin-4-one;
1-cyclopentyl-6-[(3,4-trans)-1-(2,6-dimethylbenzyl)-4-methylpyrrolidin-3-yl]-1,5-dihydro-4H-pyrazolo[3,4-d]pyrimidin-4-one;
1-cyclopentyl-6-{(3,4-trans)-1-[(4-methoxy-3,5-dimethylpyridin-2-yl)methyl]-4-methylpyrrolidin-3-yl}-1,5-dihydro-4H-pyrazolo[3,4-d]pyrimidin-4-one;
1-cyclopentyl-6-[(3,4-trans)-1-(3,5-dimethylbenzyl)-4-methylpyrrolidin-3-yl]-1,5-dihydro-4H-pyrazolo[3,4-d]pyrimidin-4-one;
1-cyclopentyl-6-[(3,4-trans)-1-(3,4-dimethylbenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(1-methyl-1H-benzimidazol-2-yl) methyl]pyrrolidin-3-yl})-1,5-dihydro-4H-pyrazolo[3,4-d]pyrimidin-4-one;
1-cyclopentyl-6-{(3,4-trans)-4-methyl-1-[(4-methyl-3,4-dihydro-2H-1,4-benzoxazin-7-yl)methyl]pyrrolidin-3-yl}-1,5-dihydro-4H-pyrazolo[3,4-d]pyrimidin-4-one;
1-cyclopentyl-6-[(3,4-trans)-4-methyl-1-(3-phenylpropyl)pyrrolidin-3-yl]-1,5-dihydro-4H-pyrazolo[3,4-d]pyrimidin-4-one;
1-cyclopentyl-6-{(3,4-trans)-4-methyl-1-[2-(trifluoromethyl)benzyl]pyrrolidin-3-yl}-1,5-dihydro-4H-pyrazolo[3,4-d]pyrimidin-4-one;
1-cyclopentyl-6-[(3,4-trans)-4-methyl-1-(4,4,4-trifluorobutyl)pyrrolidin-3-yl]-1,5-dihydro-4H-pyrazolo[3,4-d]pyrimidin-4-one;
1-cyclopentyl-6-[(3,4-trans)-1-(3-methoxy benzyl)-4-methylpyrrolidin-3-yl]-1,5-dihydro-4H-pyrazolo[3,4-d]pyrimidin-4-one;
1-cyclopentyl-6-[(3,4-trans)-1-(cyclopentylmethyl)-4-methylpyrrolidin-3-yl]-1,5-dihydro-4H-pyrazolo[3,4-d]pyrimidin-4-one;
1-cyclopentyl-6-[(3,4-trans)-1-(2,4-dimethoxybenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[4-(morpholin-4-ylmethyl)benzyl]pyrrolidin-3-yl}-1,5-dihydro-4H-pyrazolo[3,4-d]pyrimidin-4-one;
6-[(3,4-trans)-1-(2,1,3-benzothiadiazol-5-ylmethyl)-4-methylpyrrolidin-3-yl]-1-cyclopentyl-1,5-dihydro-4H-pyrazolo[3,4-d]pyrimidin-4-one;
6-{(3,4-trans)-1-[2-(benzyloxy)ethyl]-4-methylpyrrolidin-3-yl}-1-cyclopentyl-1,5-dihydro-4H-pyrazolo[3,4-d]pyrimidin-4-one;
1-cyclopentyl-6-[(3,4-trans)-1-(2,6-difluorobenzyl)-4-methylpyrrolidin-3-yl]-1,5-dihydro-4H-pyrazolo[3,4-d]pyrimidin-4-one;
1-cyclopentyl-6-[(3,4-trans)-1-(2-methoxybenzyl)-4-methylpyrrolidin-3-yl]-1,5-dihydro-4H-pyrazolo[3,4-d]pyrimidin-4-one;
1-cyclopentyl-6-{(3,4-trans)-4-methyl-1-[(3,5,6-trimethylpyrazin-2-yl)methyl]pyrrolidin-3-yl}-1,5-dihydro-4H-pyrazolo[3,4-d]pyrimidin-4-one;
1-cyclopentyl-6-[(3,4-trans)-1-(2,4-dichlorobenzyl)-4-methylpyrrolidin-3-yl]-1,5-dihydro-4H-pyrazolo[3,4-d]pyrimidin-4-one;
1-cyclopentyl-6-[(3,4-trans)-4-methyl-1-(5,6,7,8-tetrahydro-4H-pyrazolo[1,5-a]azepin-3-ylmethyl)pyrrolidin-3-yl]-1,5-dihydro-4H-pyrazolo[3,4-d]pyrimidin-4-one;
1-cyclopentyl-6-[(3,4-trans)-1-(3-fluorobenzyl)-4-methylpyrrolidin-3-yl]-1,5-dihydro-4H-pyrazolo[3,4-d]pyrimidin-4-one;
1-cyclopentyl-6-[(3,4-trans)-1-(2,3-dihydro-1-benzofuran-5-ylmethyl)-4-methylpyrrolidin-3-yl]-1,5-dihydro-4H-pyrazolo[3,4-d]pyrimidin-4-one;
1-cyclopentyl-6-[(3,4-trans)-1-(2-methoxy-5-methylbenzyl)-4-methylpyrrolidin-3-yl]-1,5-dihydro-4H-pyrazolo[3,4-d]pyrimidin-4-one;
1-cyclopentyl-6-[(3,4-trans)-1-(2-fluorobenzyl)-4-methylpyrrolidin-3-yl]-1,5-dihydro-4H-pyrazolo[3,4-d]pyrimidin-4-one;
6-[(3,4-trans)-1-(2-chlorobenzyl)-4-methylpyrrolidin-3-yl]-1-cyclopentyl-1,5-dihydro-4H-pyrazolo[3,4-d]pyrimidin-4-one;
1-cyclopentyl-6-[(3,4-trans)-1-(3,4-dichlorobenzyl)-4-methylpyrrolidin-3-yl]-1,5-dihydro-4H-pyrazolo[3,4-d]pyrimidin-4-one;
6-[(3,4-trans)-1-(2,1,3-benzothiadiazol-4-ylmethyl)-4-methylpyrrolidin-3-yl]-1-cyclopentyl-1,5-dihydro-4H-pyrazolo[3,4-d]pyrimidin-4-one;
1-cyclopentyl-6-{(3,4-trans)-4-methyl-1-[(2-propylpyrimidin-5-yl)methyl]pyrrolidin-3-yl}-1,5-dihydro-4H-pyrazolo[3,4-d]pyrimidin-4-one;
1-cyclopentyl-6-{(3,4-trans)-1-[(1-ethyl-1H-pyrazol-5-yl)methyl]-4-methylpyrrolidin-3-yl}-1,5-dihydro-4H-pyrazolo[3,4-d]pyrimidin-4-one;
1-cyclopentyl-6-{(3,4-trans)-4-methyl-1-[2-(trifluoromethoxy)benzyl]pyrrolidin-3-yl}-1,5-dihydro-4H-pyrazolo[3,4-d]pyrimidin-4-one;
1-cyclopentyl-6-{(3,4-trans)-4-methyl-1-[4-(trifluoromethyl)benzyl]pyrrolidin-3-yl}-1,5-dihydro-4H-pyrazolo[3,4-d]pyrimidin-4-one;
1-cyclopentyl-6-{(3,4-trans)-4-methyl-1-1[(1-methyl-1H-imidazo[4,5-c]pyridin-2-yl)methyl]pyrrolidin-3-yl}-1,5-dihydro-4H-pyrazolo[3,4-d]pyrimidin-4-one;
1-cyclopentyl-6-[(3,4-trans)-1-(3,5-difluorobenzyl)-4-methylpyrrolidin-3-yl]-1,5-dihydro-4H-pyrazolo[3,4-d]pyrimidin-4-one; and
1-cyclopentyl-6-[(3,4-trans)-1-(5,6-dihydro-4H-pyrrolo[1,2-b]pyrazol-3-ylmethyl)-4-methylpyrrolidin-3-yl]-1,5-dihydro-4H-pyrazolo[3,4-d]pyrimidin-4-one;
and pharmaceutically acceptable salts thereof.

In some embodiments, PDE9 inhibitor is 6-[(3S,4S)-4-methyl-1-(pyrimidin-2-ylmethyl)pyrrolidin-3-yl]-1-(tetrahydro-2H-pyran-4-yl)-1,5-dihydro-4H-pyrazolo[3,4-d]pyrimidin-4-one,

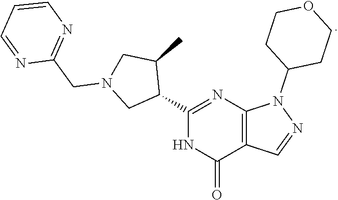 [SMILES: C[C@@H]1CN(Cc2ncccn2)C[C@H]1c1nc2c(cnn2C2CCOCC2)c(=O)[nH]1]

In some cases, as disclosed in CN108218874, PDE9 inhibitors are represented by a compound of formula (VI), or its pharmaceutically acceptable salt, solvate, polymorph and isomer,

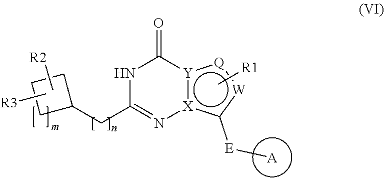

wherein,
m is arbitrarily selected from 0, 1, 2, 3 or 4;
n is arbitrarily selected from 0, 1 or 2;
X, Y, are independently selected from C, CH, and N;
Q, W are independently selected from C, CH, N, NH, S and O;
E is arbitrarily selected from —(CH₂)_{z}—, —O—, —S—, —NH—, —N(CH₃)—, wherein, z is independently selected from 0, 1 and 2;
R1 is independently selected from hydrogen, C₁₋₄ alkyl, C₂₋₄ alkenyl, C₂₋₄ alkynyl, C₃₋₆ cycloalkyl, halogenated C₁₋₄ alkyl, —(CH₂)m′-C₃₋₆ cycloalkyl, m′=1 or 2;
R2 and R3 are independently selected from hydrogen, hydroxyl, amino, carboxyl, cyano, nitro, halogen atom, C₁₋₆ alkyl, C₃₋₆ cycloalkyl, C₁₋₆ alkoxy, C₃₋₆ cycloalkyloxy, halogenated C₁₋₆ alkoxy, halogenated C₁₋₆ alkyl, halogenated C₃₋₆ cycloalkyl, C₂₋₈ alkenyl, C₂₋₈ alkyne group, amino C₁₋₆ alkyl, C₃₋₆ cycloalkylamino, C₁₋₆ alkylsulfonyl, 3-8 membered cycloalkylsulfonyl, C₁₋₆ alkylcarbonyl, C₃₋₆ Cycloalkylcarbonyl, C₁₋₆ alkylthio, —(CH₂)n′-5˜14 membered cycloalkyl, —(CH₂)n′-5˜14 membered aromatic ring, —(CH₂) n′-5-10 membered heterocyclic group containing 1 to 3 O, S and/or N atoms, —(CH₂)n′-5-10 membered heterocyclic group containing 1 to 3 O, S and/or N atoms Member heteroaryl, wherein, n′=0, 1 or 2, cycloalkyl, aromatic ring, heteroaryl ring, heterocyclyl can be optionally substituted by 1-3 R4;
R4 is independently selected from hydrogen, hydroxyl, amino, carboxyl, cyano, nitro, halogen atom, C₁₋₄ alkyl, trifluoromethyl, methoxy, cyclopropyl, —(CH₂)m′-C₃₋₆ cycloalkyl, m′=1 or 2;
Ring A is selected from optional 5-10 membered cycloalkyl, 5-10 membered aromatic ring, 5-10 membered heterocycloalkyl containing 1-3 O, S and/or N atoms, or 1-3 A 5-10 membered heteroaromatic ring of O, S and/or N atoms, wherein, ring A is optionally substituted by 1-3 R5, any ring atom S can be optionally oxidized to S(O) or S(O)₂, any ring atom carbon can be optionally oxidized to C(O);
R is independently selected from hydrogen, hydroxyl, amino, carboxyl, cyano, nitro, halogen atom, C₁₋₆ alkyl, C₁₋₆ alkoxy, methoxy, trifluoromethyl, cyclopropyl, —(CH₂)m′—C₃₋₆ cycloalkyl, m′=1 or 2.

In some cases, as disclosed in US20220160696 and WO2020182076, PDE9 inhibitors are represented by a compound of formula (VII),

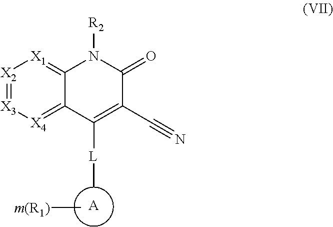

wherein, X₁, X₂, X₃, and X₄ are independently selected from CR₃ or N, and N heteroatoms can be optionally oxidized to

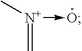

R₃ at each occurrence is independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halo C₁₋₆ alkyl, halo C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂ amino, halo C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy are not substituted or optionally substituted with one or more groups independently selected from hydroxyl, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₂₋₈ alkynyl, halo C₁₋₆ alkyl, C₂₋₈ alkenyl, halo C₁₋₆ alkoxy, 4-6 membered heterocyclyl unsubstituted or optionally substituted with a substituent, and heteroaryl unsubstituted or optionally substituted with a substituent;
the substituents of the above-mentioned 4-6 membered heterocyclyl optionally substituted with a substituent and heteroaryl optionally substituted with a substituent are selected from hydroxyl, amino, carboxyl, cyano, nitro, halogen, C₁₋₆ alkyl and C₁₋₆ alkoxy;
L is a bond and —NH—(CH₂)t-, and t is 0, 1, 2 or 3;
ring A is 3-12 membered heterocyclyl, aryl, 5-10 membered heteroaryl, 3-12 membered cycloalkyl, and 3-12 membered cycloalkenyl, wherein the heteroatom of the 3-12 membered heterocyclyl is selected from one of O, S, and N or any combination thereof, the S atom may be optionally oxidized to S(O) or S(O)₂, the C atom may be optionally oxidized to C(O), and the N heteroatom may be optionally oxidized to

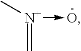

and the heteroatom of the 5-10 membered heteroaryl is selected from one of O, S and N or any combination thereof;
each R₁ is independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halo C₁₋₆ alkyl, halo C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halo C₁₋₆ alkyl, halo C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are not substituted or optionally substituted with groups selected from hydroxyl, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ alkylsulfonylamino;
m is 0, 1, 2 or 3; and
R² is selected from hydrogen, C₁₋₆ alkyl, C₂₋₈ alkenyl, C₂₋₈ alkynyl, and halo C₁₋₆ alkyl.

In some cases, as disclosed in CN108341819, PDE9 inhibitors are represented by a compound of formula (VIII) or its is pharmaceutically acceptable salt, solvated compounds, polymorph and isomers;

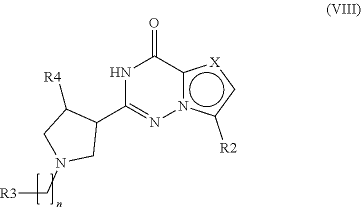

wherein,
n is selected from 0, 1 or 2;
X is selected from N or CR₁;
R₁ is independently selected from hydrogen, C₁₋₆Alkyl, C₂₋₈Alkenyl, C₂₋₈Alkynyl, C₃₋₆ naphthenic base, halogenated C₁₋₆Alkyl, —(CH₂)m′-C₃₋₆naphthenic base, m′=1 or 2;
R₂ is independently selected from hydrogen, C₁₋₆Alkyl, C₁₋₆Alkoxy, halogenated C₁₋₆Alkyl, halogenated C₁₋₆Alkoxy, C₃₋₆Naphthenic base Oxygroup, C₃₋₆Cycloalkyl amino, halogenated C₃₋₆Naphthenic base, C₂₋₈Alkenyl, C₂₋₈Alkynyl, C₁₋₆Alkyl sulphonyl, C₃₋₈First naphthenic base sulphur Acyl group, C₁₋₆Alkyl sulfenyl, C₂₋₈Alkenyl (C₃₋₆) naphthenic base, C₂₋₈Alkynyl (C₃₋₆) naphthenic base, C₁₋₆Alkyl-carbonyl, C₃₋₆Naphthenic base Carbonyl, —(CH₂)_{n′}-3-14 members naphthenic base, —(CH₂)_{n′}—5-14 members aromatic ring, —(CH₂)_{n′}—5-10 circle heterocyclic rings base, —(CH₂)_{n′}-5-10 unit's heteroaryls, the heterocycle, heteroaryl contain 1-3 O, S and/or N atom, wherein the arbitrary annular atom of heterocycle, heteroaryl Sulphur can optionally be oxidized to S (O) or S(O)₂, arbitrary annular atom carbon is optionally oxidized to C (O), n′=0, 1 or 2, alkyl ring, heterocycle, aromatic ring, hetero-aromatic ring can be optionally by 1-3 R₅;
wherein, when X is selected from CR1, and R1 is hydrogen, R2 cannot be

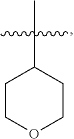 [SMILES: *C1CCOCC1]

R³ is independently selected from
hydrogen, hydroxyl, amino, carboxyl, cyano, nitro, halogen atom, C₁₋₆ alkyl, C₃₋₆ cycloalkyl, C₁₋₆ alkoxy, C₃₋₆ Cycloalkyloxy, halogenated C₁₋₆ alkoxy, halogenated C₁₋₆ alkyl, halogenated C₃₋₆ cycloalkyl, C₂₋₈ alkenyl, C₂₋₈ alkynyl, amino C₁₋₆ alkyl, C₃₋₆ cycloalkylamino, C₁₋₆ alkylsulfonyl, 3-8 membered cycloalkylsulfonyl, C₁₋₆ alkylcarbonyl, C₃₋₆ cycloalkyl Carbonyl, C₁₋₆ alkylthio, 3-14 membered cycloalkyl, 5-14 membered aromatic ring, -5-10 membered heterocyclic group, -5-10 membered heteroaryl group, said heterocyclic group, the heteroaryl group contains 1-3 O, S and/or N atoms, wherein, any ring atom sulfur of the heterocyclic group and the heteroaryl group can be optionally oxidized to S(O) or S(O)₂, any ring atomic carbon can be optionally oxidized to C(O), cycloalkyl, aromatic ring, heteroaryl ring, heterocyclyl can be optionally substituted by 1-3 R₆;
R₄ is independently selected from hydrogen, hydroxyl, amino, carboxyl, cyano, nitro, halogen atom, C₁₋₆alkyl, C₁₋₆alkoxy, C₃₋₆cycloalkyl oxy, halogenated C₁₋₆alkoxy, halogenated C₁₋₆alkyl, halogenated C₃₋₆naphthenic base, C₂₋₆alkenyl, C₂₋₆alkynyl, amino C₁₋₆ alkyl, C₃₋₆cycloalkyl amino, C₁₋₆alkyl sulphonyl, 3-8 membered rings alkyl sulphonyl, C₁₋₆alkyl-carbonyl or C₃₋₆Naphthenic base carbonyl Base, C₁₋₆alkyl sulfenyl or —(CH₂)_{n′}—3-7 members naphthenic base, —(CH₂)_{n′}—5-6 members aromatic ring, —(CH₂)_{n′}-5-7 circle heterocyclic rings base, —(CH₂)_{n′}-5-6 unit's heteroaryls, the heterocycle, heteroaryl contain 1-3 O, S and/or N atom, wherein heterocycle, heteroaryl Arbitrary ring S can optionally be oxidized to S(O) or S(O)₂, arbitrary ring carbon is optionally oxidized to C(O), n′=0, 1 or 2, ring Alkyl, aromatic ring, hetero-aromatic ring, heterocycle can be optionally by 1-3 R₇;
R₅ is independently selected from hydrogen, hydroxyl, amino, carboxyl, cyano, nitro, halogen atom, trifluoromethyl, methoxyl group, —(CH₂)_{n′}-3-14 members naphthenic base, —(CH₂)_{n′}—5-14 members aromatic ring, —(CH₂)_{n′}-5-10 circle heterocyclic rings base, —(CH₂)_{n′}—5-10 members Heteroaryl, the heterocycle, heteroaryl contain 0-3 O, S and/or N atom, wherein n′=0, 1 or 2;
R₆ and R₇ are independently selected from hydrogen, hydroxyl, amino, carboxyl, cyano, nitro, halogen atom, trifluoromethyl, C₁₋₆Alkyl, C₁₋₄Alkoxy, C₁₋₆Alkyl sulphonyl, C₁₋₆Alkyl sulphonyl C₁₋₆Alkyl, C₁₋₆Alkyl sulphonyl C₁₋₆Alkoxy, aminosulfonyl Amino C₁₋₆Alkyl, methoxyl group, cyclopropyl, —(CH₂)m′—C₃₋₆Naphthenic base, m′=1 or 2.

In some cases, as disclosed in U.S. Ser. No. 10/858,362, U.S. Ser. No. 11/472,810, and WO2016174188, PDE9 inhibitors are represented by a compound of formula (X),

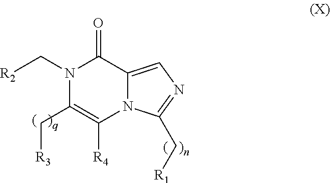

wherein:
   n is 0 or 1;
   g is 0 or 1;
   R₁ is selected from the group consisting of benzyl, indanyl, indoline and 5-membered heteroaryls; all of which can be substituted with a substituent selected from the group consisting of halogen and C₁-C₃ alkyl; or
   R₁ is selected from the group consisting of saturated monocyclic rings containing 4-6 carbon atoms and 1-2 nitrogen atoms; all of which can be substituted one or more times with one or more substituents selected from the group consisting of methyl, fluorine and sulfonamide; or
   R₁ is selected from the group consisting of lactams containing 4-6 carbon atoms; all of which can be substituted one or more times with one or more substituents selected from the group consisting of methyl and fluorine; or
   R₁ is selected from the group consisting of bicyclic ethers such as, 7-oxabicyclo[2.2.1]heptane; all of which can be substituted one or more times with one or more substituents selected from the group consisting of methyl and fluorine; or
   R₁ is selected from the group consisting of linear or branched C₁-C₈ alkyl, saturated monocyclic C₃-C₅ cycloalkyl, oxetanyl, tetrahydrofuranyl and tetrahydropyranyl; all of which can be substituted one or more times with one or more substituents selected from the group consisting of methyl, fluorine, hydroxy, cyano or methoxy; or
   R₁ is a linear or branched C₁-C₃ alkyl, which is substituted with a substituent selected from phenyl and 5-membered heteroaryl, wherein said 5-membered heteroaryl can be substituted with one or more C₁-C₃ alkyls; or
   R₁ is selected from the group consisting of morpholine, tetrahydrofuran-3-amine, hexahydro-2H-furo[3,2-b]pyrrole and homomorpholine; all of which can be subsituted with one or more substituents selected from the group consisting of C₁-C₃ alkyl;
   R₂ is selected from the group consisting of hydrogen, linear or branched C₁-C₈ alkyl, phenyl, saturated monocyclic C₃-C₈ cycloalkyl, oxetanyl, benzo[d][1,3]dioxolyl, tetrahydrofuranyl and tetrahydropyranyl; or
   R₂ is phenyl or pyridyl substituted with one or more substituents selected from the group consisting of hydroxyl, amino, cyano, halogen, C₁-C₃ alkyl, C₁-C₃ alkoxy, C₃-C₅ cycloalkoxy, C₃-C₅ cycloalkyl-methoxy, C₁-C₃ fluoroalkoxy, and —NC(O)CH₃; or
   R₂ is a 5-membered heteroaryl which can be substituted one or more times with C₁-C₃ alkyl;
   R₃ is selected from the group consisting of hydrogen, halogen, C₁—C alkyl, C₃-C₅ cycloalkyl and phenyl; or
   R₃ is selected from the group consisting of phenyl substituted one or more times with C₁-C₃ alkyl; methyl substituted one, two or three times with fluorine; ethyl substituted one, two or three times with fluorine;
   R₄ is hydrogen;
and tautomers and pharmaceutically acceptable addition salts thereof;
with the proviso that R2 and R3 cannot be hydrogen at the same time;
with the proviso, that the compound of formula (I) is not one of the three following compounds:
3-methyl-7-(4-(trifluoromethoxy)benzyl)imidazo[1,5-a]pyrazin-8(7H)-one;
7-butyl-3-methylimidazo[1,5-a]pyrazin-8(7H)-one; and
7-(4-methoxybenzyl)-3-methylimidazo[1,5-a]pyrazin-8(7H)-one.

In some cases, as disclosed in U.S. Pat. Nos. 9,725,453, 9,969,742, and WO2013142269, PDE9 inhibitors are represented by a compound of formula (XI),

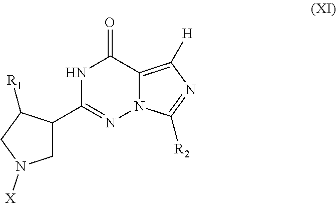

wherein:
X is selected from the group consisting of a bond, C(O) and S(O)₂;
R₁ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and
heterocycloalkyloxy, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl];
R₂ is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl]; and
R₃ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl].

In some cases, as disclosed in US20110212960 and WO2011018495, PDE9 inhibitors are represented by a compound of formula (XII),

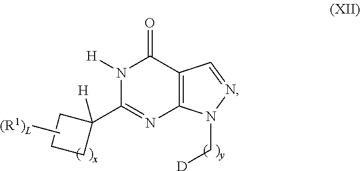

wherein
   R¹ is selected independently for each R¹ from the group R^{1a} consisting of Hydrogen, fluorine, chlorine, bromine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, heterocyclyl-CO—, R¹⁰—O—, R¹⁰—O—C₁₋₃-alkyl-, (R¹⁰)₂N—, R¹⁰—CO—, (R⁹)₂N—CO—, R¹⁰—CO—(R¹⁰)N—, R¹⁰—CO—, (R⁹)₂N—CO—(R¹⁰)N—, (R⁹)₂N—CO—O—, R¹⁰—O—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, heterocyclyl-CO—, and C₁₋₆-alkyl-SO₂— may optionally be substituted independently of one another by one or more substituents selected independently of one another from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, C₃₋₆-cycloalkyl-C₁₋₄-alkyl-, and C₁₋₆-alkyl-, preferably from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, and C₃₋₆-cycloalkyl-C₁₋₄-alkyl-,
L is selected from the integers 0, 1, 2 and 3,
x is selected from the integers 0, 1, 2, 3 and 4,
y is selected from the integers 0, 1 and 2,
D is selected from the group D^{1a} consisting of heterocyclyl,
   wherein the above-mentioned members of the group D^{1a} may optionally be substituted by one or more substituents selected independently of one another from the group R² and/or optionally substituted by one group R³ or
D is selected from the group D^{2a} consisting of cyclopropyl, cyclobutyl, cyclopentyl, cyclohexyl, cycloheptyl cyclooctyl, cyclobutenyl, cyclopentenyl, cyclohexenyl, cycloheptenyl, cyclooctenyl, cyclopentadienyl, cyclohexadienyl, cycloheptadienyl, cyclooctadienyl, cycloheptatrienyl, cyclooctatrienyl and cyclooctatetraenyl,
   wherein the above-mentioned members of the group D^{2a} may optionally be substituted by one or more substituents selected independently of one another from the group R⁴,
   or
D is selected from the group D^{3a} consisting of C₁₋₈-alkyl
   wherein the above-mentioned C₁₋₈-alkyl-group D^{3a} may optionally be substituted by one or more substituents selected independently of one another from the group R⁵.
   or
D is selected from the group D⁴a consisting of aryl
   wherein the above-mentioned aryl group D^{4a} may optionally be substituted by one or more substituents selected independently of one another from the group consisting of R⁶. Preferred are such compounds wherein D^{4a} is substituted by not more than one R⁶.
   or
D is selected from the group D^{5a} consisting of heteroaryl
   wherein the above-mentioned members of the group D^{5a} may optionally be substituted by one or more substituents selected independently of one another from the group R⁶. Preferred are such compounds wherein D⁵a is substituted by not more than one R⁶.
R² is selected from the group R^{2a} consisting of
   H—, fluorine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, C₁₋₆-alkyl-SO₂—, and oxo,
   where the above-mentioned members HF₂C—, FH₂C—, F₃C—CH₂—, C₁₋₆-alkyl-(Preferably C₂₋₆-alkyl), C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—C₁₋₃-alkyl-, and C₁₋₆-alkyl-SO₂— may optionally be substituted independently of one another by one or more substituents selected independently of one another from the group consisting of
   fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰₂N—, (R¹⁰)₂N—C₁₋₃-alkyl-, and (R¹⁰)₂N—CO—, and in cases in that D¹ is a heterocyclyl group with NR² as ring member, R² shall be
      independently of any other R²: H—, F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, R¹⁰—CO—, (R¹⁰)₂N—CO—, R¹⁰—CO—, R¹⁰—SO₂—, and C₁₋₆-alkyl-SO₂—, where the above-mentioned members F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, and C₁₋₆-alkyl-SO₂— may optionally be substituted independently of one another by one or more substituents selected independently of one another from the group consisting of
      fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and (R¹⁰)₂N—CO—,
R³ is selected from the group R^{3a} consisting of
   H—, HO— and R¹⁰—O—,
R⁴ is selected from the group R^{4a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁-6-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
   or
   two substituents R^{4a} together form a C₂₋₆-alkylene bridge, wherein one or two CH₂ groups of the C₂₋₆-alkylene bridge may be replaced independently of one another by O, S, SO, SO₂, N(R¹⁰) or N—C(O)—R¹⁰ in such a way that in each case two O or S atoms or an 0 and an S atom are not joined together directly.
   R⁵ is selected from the group R^{5a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—, where the above-mentioned members HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂—, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁶ is selected from the group R^{6a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁-6-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl- and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁹ is selected independently for each R⁹ from the group R^{9a} consisting of H—F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, and
   where the above-mentioned members F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, may optionally be substituted independently of one another by one or more substituents selected independently of one another from the group consisting of
   fluorine, chlorine, bromine, HO—, NC—, O₂N—, F₃C—, HF₂C—, FH₂C—, F₃C—CH₂—, HO—C₁₋₆-alkyl-, CH₃—O—C-6-alkyl-, C₁₋₆-alkyl-O— and C₁₋₆-alkyl-,
R¹⁰ is selected independently for each R¹⁰ from the group R^{10a} consisting of H— (but not in case is part of a group being selected from R¹⁰O—CO—, R¹⁰—SO₂— or R¹⁰—CO—), F₃C—CH₂—, C₁₋₆-alkyl-, C₂₋₆-alkenyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, and heteroaryl-C₁₋₃-alkyl-,
   and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —S—, —NH—, —N(C₃₋₆-cycloalkyl)-, —N(C₃₋₆-cycloalkyl-C₁₋₄-alkyl)- or —N(C₁₋₄-alkyl)- and
   where the above-mentioned members may optionally be substituted independently of one another by one or more substituents selected from the group consisting of
   fluorine, chlorine, bromine, HO—, NC—, O₂N—, F₃C—, HF₂C—, FH₂C—, F₃C—CH₂—, HO—C₁₋₆-alkyl-, CH₃—O—C-6-alkyl-, C₁₋₆-alkyl- and C₁₋₆-alkyl-O—,
   and pharmaceutically acceptable salt forms or solvates thereof.

In some cases, as disclosed in U.S. Pat. No. 8,809,345 and WO2012110441, PDE9 inhibitors are represented by a compound of formula (XIII),

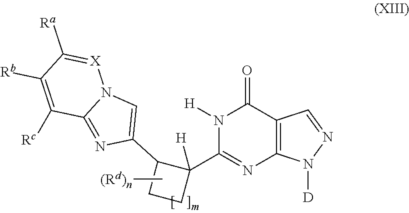

wherein
X is N or CR^{c},
R^{a}, R^{b}, R^{c}, R^{e} are selected independently of each other from the group consisting of H, C₁₋₆alkyl-, C₁₋₆-alkyl-O—, CF₃—, CHF₂—, CH₂F—, NC— and halogen; whereby C₁₋₆alkyl- and C₁₋₆-alkyl-O—
optionally may be substituted with halogen, preferably with fluoro
R^{d} is selected from the group consisting of fluorine, NC—, —CF₃, —CHF₂, —CH₂F, and methyl; D is selected from the group consisting of cyclopentyl, cyclohexyl, tetrahydrofuranyl, tetrahydropyranyl, 2-, 3- and 4-pyridyl,
whereby cyclopentyl and cyclohexyl optionally may be substituted by 1 or 2 substituents, whereby said substituents may be selected independently of one another from the group consisting of fluorine, NC—, F₃C—, HF₂C— and FH₂C—;
whereby tetrahydrofuranyl and tetrahydropyranyl optionally may be substituted by 1 or 2 substituents, whereby said substituents may be selected independently of one another from the group consisting of fluorine, NC—, F₃C—, FIF₂C— and FH₂C—;
whereby pyridyl optionally may be substituted by 1, 2, 3 or 4 substituents, whereby said substituents may be selected independently of one another from the group consisting of fluorine, chlorine, bromine, NC—, F₃C—, HF₂C—, FH₂C—; F₃C—CH₂—, C₁₋₆-alkyl- and C₃₋₇-cycloalkyl;
m: is selected from 1 or 2, preferably 1;
n: is selected from 0, 1 or 2, preferably, 0 or 1, more preferably 0,
whereby if n=2, the two groups R^{d} are selected independently of one another; and salts, preferably pharmaceutically acceptable salts thereof, solvates thereof and the solvates of the aforementioned salts thereof.

In some cases, as disclosed in U.S. Pat. Nos. 7,615,558, 8,809,348, and WO2004099210, PDE9 inhibitors are represented by a compound of formula (XIV),

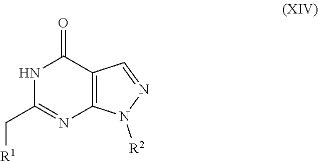

in which
R¹ is phenyl, pyridyl or thiophenyl, which are optionally selected with up to 3 substituents independently of one another from the group of C₁-C₆-alkyl, C₁-C₆-alkoxy, hydroxycarbonyl, cyano, trifluoromethyl, amino, nitro, hydroxy, C₁-C₆Alkylamino, halogen,
C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-Akylsulfonylamino, C₁-C₆ Alkylsulfonyl, C₁-C₆ alkylthio, wherein C₁-C₆-alkyl, C₁-C₆-alkoxy, C₆-C₁₀ alkylamino, C₆-C₁₀-Axylcarbonylamino, C₁-C₆-Alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulfonylamino, C₁-C₆-alkylsulfonyl and C₁-C₆-alkylthio optionally with a residue Group hydroxy, cyano, halogen, hydroxy, carbonyl and a group of the formula —NR³R⁴,
in which
R³ and R⁴ independently of one another are hydrogen or C₁-C₆-alkyl,
or
R³ and R⁴ together with the nitrogen atom to which they are attached, 5- to 8-membered heterocyclyl,
R² is phenyl or heteroaryl, phenyl having 1 to 3 residues and heteroaryl optionally having 1 to 3 residues, each independently selected from the group C₁-C₆ alkyl, C₁-C₆ alkoxy, hydroxycarbonyl, cyano, trifluoromethyl, amino, nitro, hydroxy, C₁-C₆ alkylamino, halogen, C₆-C₁₀ arylcarbonylamino, C₁-C₆ alkylcarbonylamino, C₁-C₆ alkylaminocarbonyl, C₁-C₆ alkoxycarbonyl, C₆-C₁₀ arylaminocarbonyl, heteroarylaminocarbonyl, heteroarylcarbonylamino, C₁-C₆ alkylsulfonylamino, and C₁-C₆-alkylthio, wherein C₁-C₆-alkyl, C₁-C₆-alkoxy, C₁-C₆ alkylamino, C₆-C₁₀-arylcarbonylamino, C₁-C₆-alkylcarbonylamino, C₁-C₆ alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulfonylamino, C₁-C₆ alkylsulfonyl and C₁-C₆ alkylthio optionally with a radical selected from the group consisting of hydroxy, cyano, halogen, hydroxycarbonyl and a group of the formula —NR³R⁴,
where R³ and R⁴ have the meanings given above,
or their salts, solvates and/or solvates of the salts.

In some cases, as disclosed in U.S. Pat. No. 8,158,633, US20120165349, and WO2004018474, PDE9 inhibitors are represented by a compound of formula (XV),

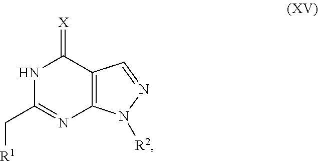

in which
   R¹ is phenyl which is substituted by 1 to 5 substituents independently of one another selected from the group of halogen, C₁-C₆-alkyl, trifluoromethyl, trifluoromethoxy, cyano, hydroxy, nitro and C₁-C₆-alkoxy,
   R² is pentan-3-yl, C₄-C₆-cycloalkyl,
   X is oxygen or sulfur,
   and the salts, solvates and/or solvates of the salts thereof.

In some cases, as disclosed in U.S. Pat. Nos. 8,039,477, 8,741,907, and WO2004026876, PDE9 inhibitors are represented by a compound of formula (XVI),

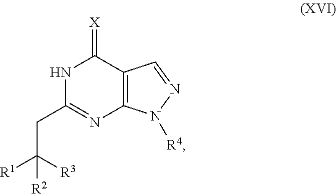

in which
   R¹ is C₁-C₆-alkyl, trifluoromethyl, hydroxy, C₁-C₆-alkoxy, —C(═O)OR⁵ or —C(═O)NR⁶R⁷, where C₁-C₆-Alkyl is optionally substituted by 1 to 3 radicals independently of one another selected from the group of hydroxy, C₁-C₆-alkoxy, halogen, trifluoromethyl, trifluoromethoxy, —C(═O)OR⁵ or —C(═O)NR⁶R⁷, and
      R⁵ is C₁-C₆-alkyl,
      R⁶ and R⁷ are independently of one another hydrogen, C₆-C₁₀-aryl, C₁-C₆-alkyl, or
         together with the nitrogen atom to which they are bonded form a 4- to 10-membered heterocyclyl,
   R² is hydrogen, C₁-C₆-alkyl, trifluoromethyl, C₁-C₆-alkoxy,
   or
   R¹ and R² together with the carbon atom to which they are bonded form C₃-C₈-cycloalkyl, C₃-C₈-cycloalkenyl or 4- to 10-membered heterocyclyl, which are optionally substituted by up to 2 substituents from the group of C₁-C₆-alkyl, C₁-C₆-alkoxy, hydroxy, oxo, —C(═O)OR^{B}, and R⁸ is C₁-C₆-alkyl or benzyl,
   R³ is hydrogen or C₁-C₆-alkyl,
   R⁴ is pentan-3-yl, C₃-C₆-cycloalkyl,
   X is oxygen or sulfur,
   and the salts, solvates and/or solvates of the salts thereof.

In some cases, as disclosed in U.S. Pat. No. 8,648,085 and WO2009068617, PDE9 inhibitors are represented by a compound of formula (XVII),

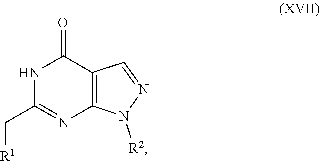

with:
   R¹
   being phenyl or pyridyl, any of which is substituted with 1 to 4 substituents X;
   and with the option that each of phenyl or pyridyl in addition may be substituted by up to 3 radicals independently of one another selected from the group of C₁-C₆-alkyl, C₁-C₆-alkoxy, hydroxycarbonyl, cyano, trifluoromethyl, amino, nitro, hydroxy, C₁-C₆-alkylamino, halogen, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylamino-carbonyl, heteroarylcarbonylamino, C₁-C₆-alkylsulphonylamino, C₁-C₆-alkylsulphonyl, or C₁-C₆-alkylthio,
   where each of C₁-C₆-alkyl, C₁-C₆-alkoxy, C₁-C₆-alkylamino, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio are optionally substituted by 1 to 3 radicals independently of one another selected from the group of hydroxy, cyano, halogen, hydroxycarbonyl and a group of the formula —NR³R⁴,
   X
   independently of each other being selected from C₁-C₆-alkoxy, where the C₁-C₆-alkoxy is substituted with 2 to 6 halogen substituents, and the halogen substituents are selected from the group of fluoro, chloro and bromo, whereby the C-atom directly attached to the 0-atom, which constitutes the beta position with respect to the link to the phenyl or pyridyl, is substituted with at least one halogen atom;
   R²
   being phenyl or heteroaryl, where phenyl is substituted by 1 to 3 radicals and heteroaryl is optionally substituted by 1 to 3 radicals in each case independently of one another selected from the group of C₁-C₆-alkyl, C₁-C₆-alkoxy, hydroxycarbonyl, cyano, trifluoromethyl, amino, nitro, hydroxy, C₁-C₆-alkylamino, halogen, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio,
   where each of C₁-C₆-alkyl, C₁-C₆-alkoxy, C₁-C₆-alkylamino, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio are optionally substituted by one to three radicals independently of one another selected from the group of hydroxy, cyano, halogen, hydroxycarbonyl and a group of the formula —NR³R⁴,
   R³
   being hydrogen or C₁-C₆-alkyl,
   and R⁴
   being hydrogen or C₁-C₆-alkyl,
   or R³ and R⁴ together with the nitrogen atom to which they are bonded are 5- to 8-membered heterocyclyl.

In some cases, as disclosed in U.S. Pat. Nos. 9,096,603, 8,623,879, and WO2009121919, PDE9 inhibitors are represented by a compound of formula (XVIII),

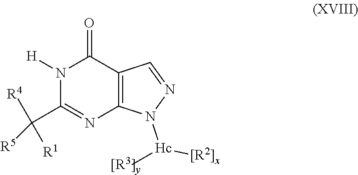

Hc is selected from the group of tetrahydropyranyl, tetrahydrofuranyl, piperidinyl and pyrrolidinyl;
R¹ is selected from the group of phenyl, 2-, 3- and 4-pyridyl-, pyrimidinyl, pyrazolyl, thiazolyl, cyclopropyl, cyclobutyl, cyclopentyl, cycloheptyl, cyclopentylmethyl, cyclohexyl, ethyl, 1- and 2-propyl, 1- and 2-butyl-, 1-, 2- and 3-pentyl-, tetrahydrofuranyl and tetrahydropyranyl,
where these R¹ groups may optionally be substituted by one or more substituents independently of one another selected from the group consisting of fluorine, chlorine, bromine, iodine, HO—, oxo, NC—, C₁₋₆-alkyl-O—, C₁₋₆-alkyl-, C₃₋₇-cycloalkyl-O—, C₃₋₇cycloalkyl-C₁₋₃alkyl-O—, CF₃O— and CF₃;
R² independently of any other R² is selected from the group of H—, C₁₋₆-alkyl- and oxo, and in cases wherein R² is attached to a nitrogen which is a ring member of Hc, this R² shall be independently of any other R² selected from: H—, C₁₋₆-alkyl-CO—, C₁₋₆-alkyl-O—CO—, C₁₋₆-alkyl-, phenyl-CO—, phenyl-O—CO— and (C₁₋₆-alkyl)₂N—CO—, where the above-mentioned R² members may optionally be substituted independently of one another by one or more fluorine substituents;
R³ is selected from the group of H—, hydroxy and C₁₋₆-alkyl-O—, whereby C₁₋₆-alkyl-O— may optionally be substituted by one or more fluorine, chlorine, bromine and HO—;
R⁴ and R⁵ independently of one another are selected from the group of H—, fluorine, and methyl;
x is 0, 1, 2, 3 or 4;
y is 0 or 1;
or a pharmaceutically acceptable salt, a tautomer or a stereoisomer thereof.

In some embodiments, the PDE 9 inhibitor is selected from the group consisting of

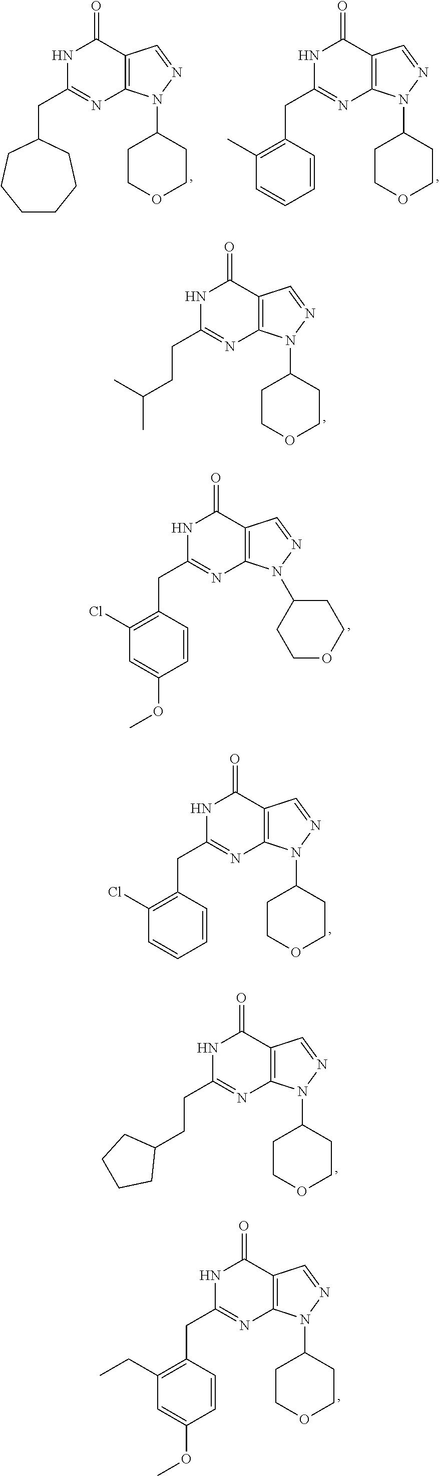

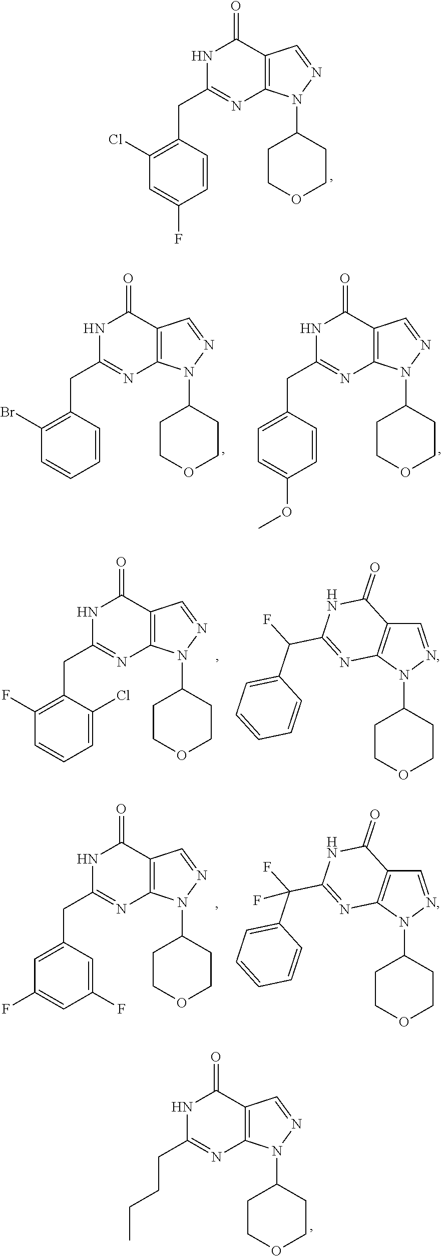

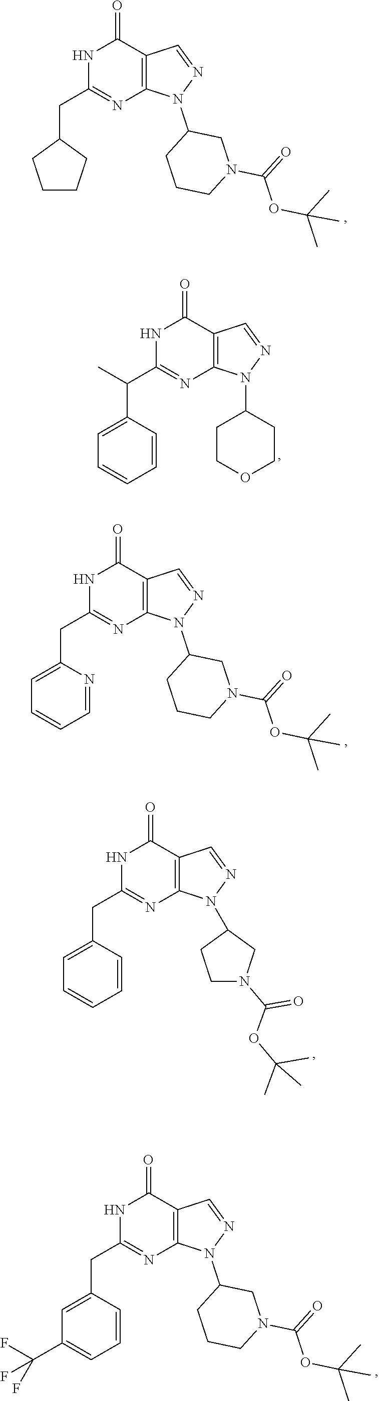

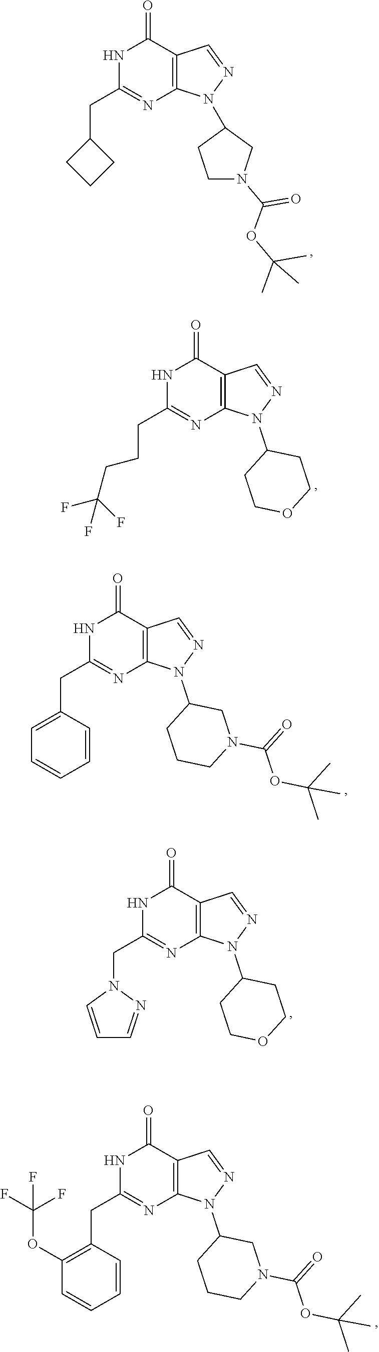 [SMILES: CC(C)(C)OC(=O)N1CCC(n2ncc3c(=O)[nH]c(CC4CCC4)nc32)C1 ; O=c1[nH]c(CCCC(F)(F)F)nc2c1cnn2C1CCOCC1 ; CC(C)(C)OC(=O)N1CCCC(n2ncc3c(=O)[nH]c(Cc4ccccc4)nc32)C1 ; O=c1[nH]c(Cn2cccn2)nc2c1cnn2C1CCOCC1 ; CC(C)(C)OC(=O)N1CCCC(n2ncc3c(=O)[nH]c(Cc4ccccc4OC(F)(F)F)nc32)C1]

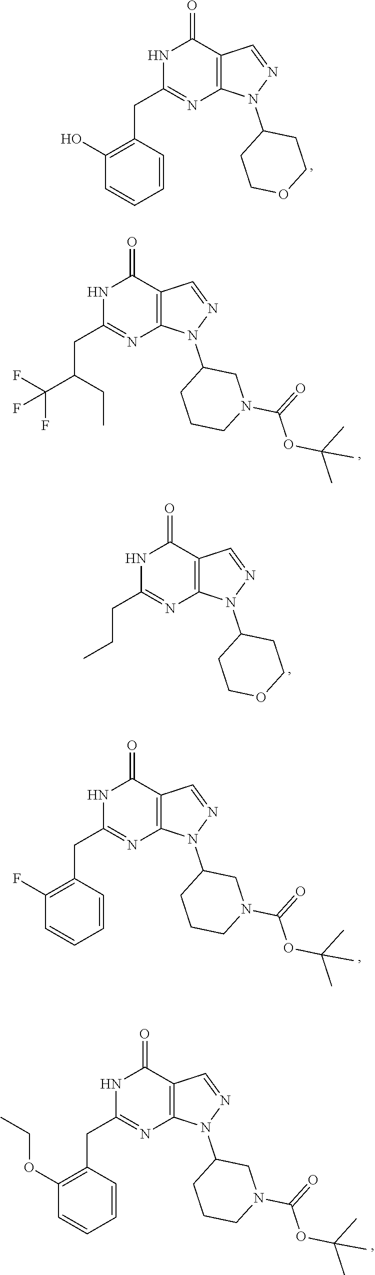

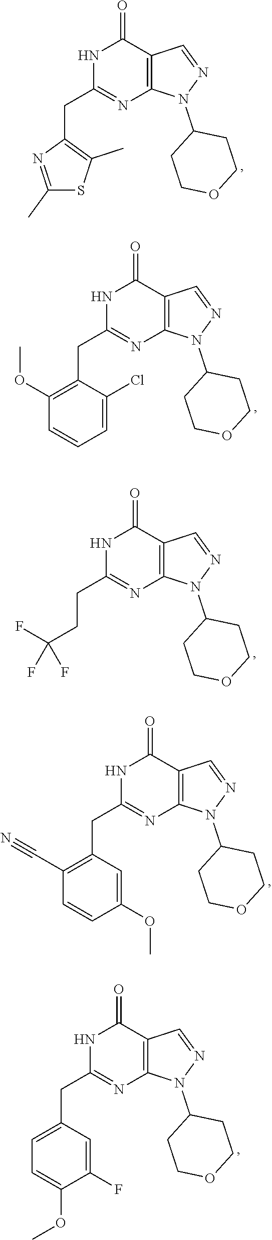 [SMILES: Cc1nc(Cc2nc3c(cnn3C3CCOCC3)c(=O)[nH]2)c(C)s1 ; COc1cccc(Cl)c1Cc1nc2c(cnn2C2CCOCC2)c(=O)[nH]1 ; O=c1[nH]c(CCC(F)(F)F)nc2c1cnn2C1CCOCC1 ; COc1ccc(C#N)c(Cc2nc3c(cnn3C3CCOCC3)c(=O)[nH]2)c1 ; COc1ccc(Cc2nc3c(cnn3C3CCOCC3)c(=O)[nH]2)cc1F]

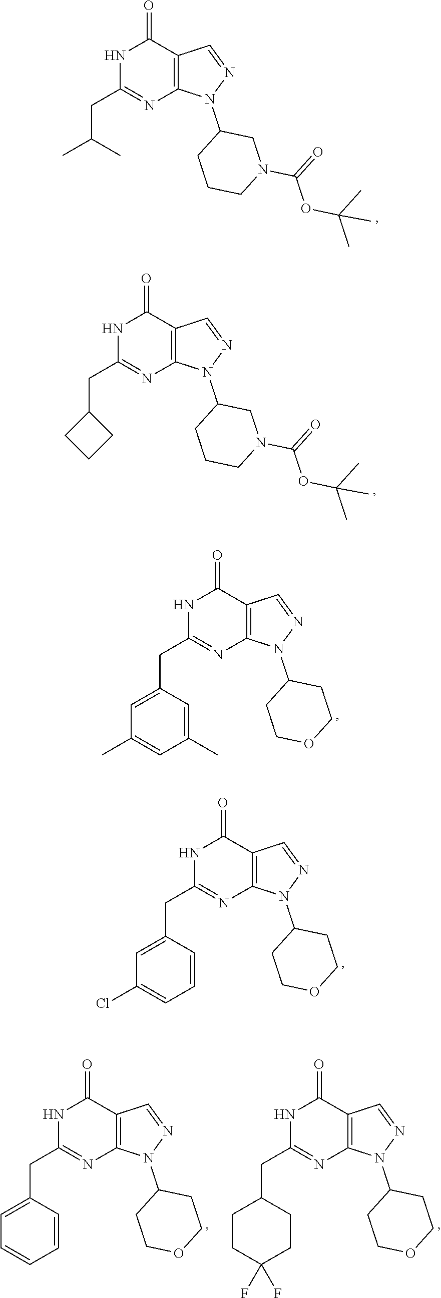

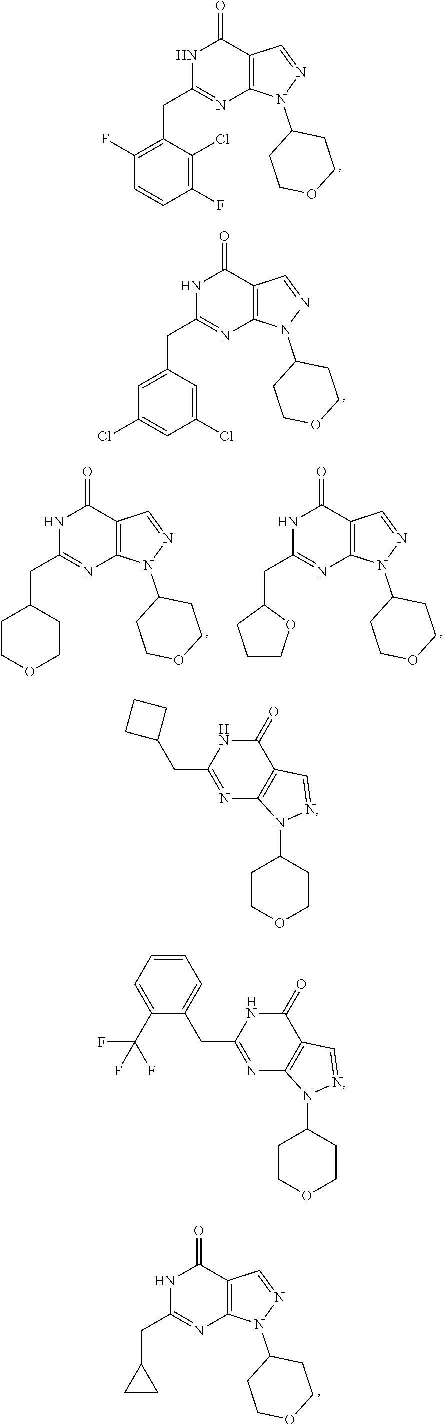

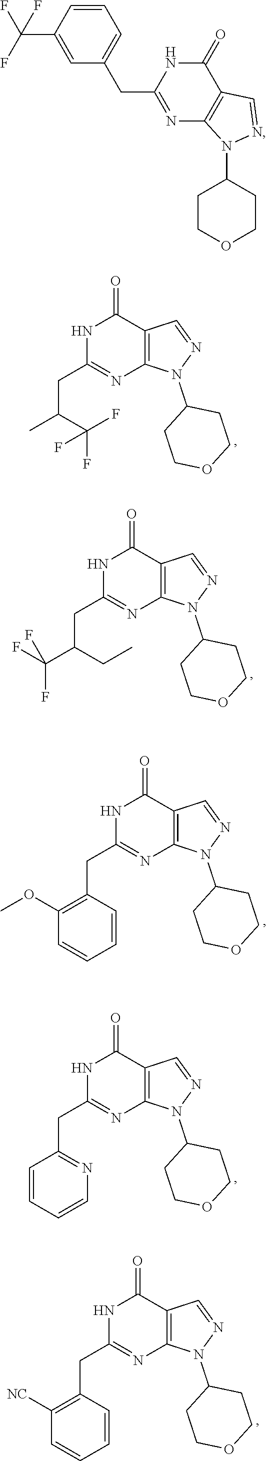 [SMILES: O=c1[nH]c(Cc2cccc(C(F)(F)F)c2)nc2c1cnn2C1CCOCC1 ; CC(Cc1nc2c(cnn2C2CCOCC2)c(=O)[nH]1)C(F)(F)F ; CCC(Cc1nc2c(cnn2C2CCOCC2)c(=O)[nH]1)C(F)(F)F ; COc1ccccc1Cc1nc2c(cnn2C2CCOCC2)c(=O)[nH]1 ; O=c1[nH]c(Cc2ccccn2)nc2c1cnn2C1CCOCC1 ; N#Cc1ccccc1Cc1nc2c(cnn2C2CCOCC2)c(=O)[nH]1]

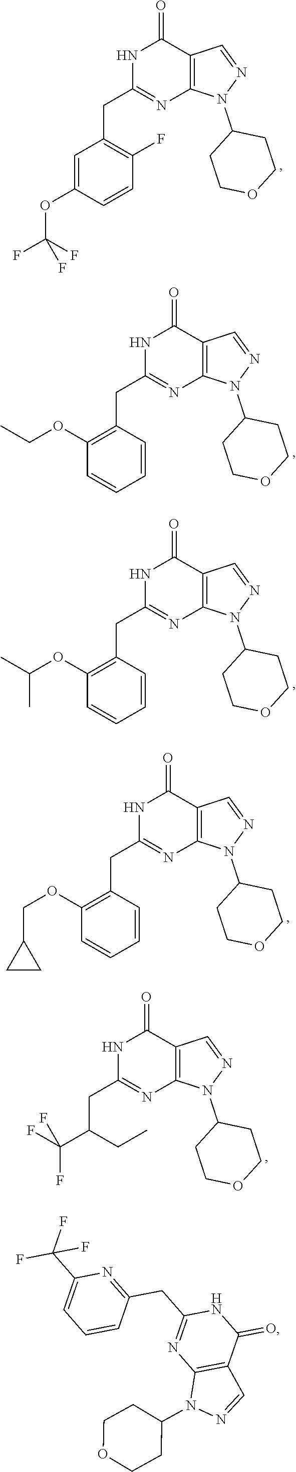

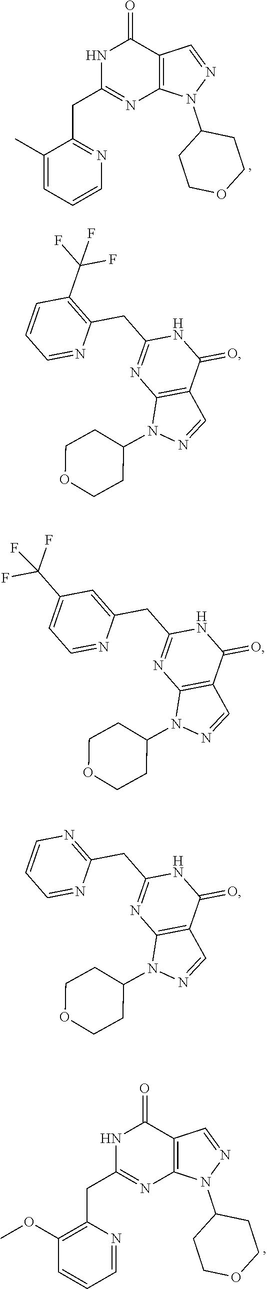

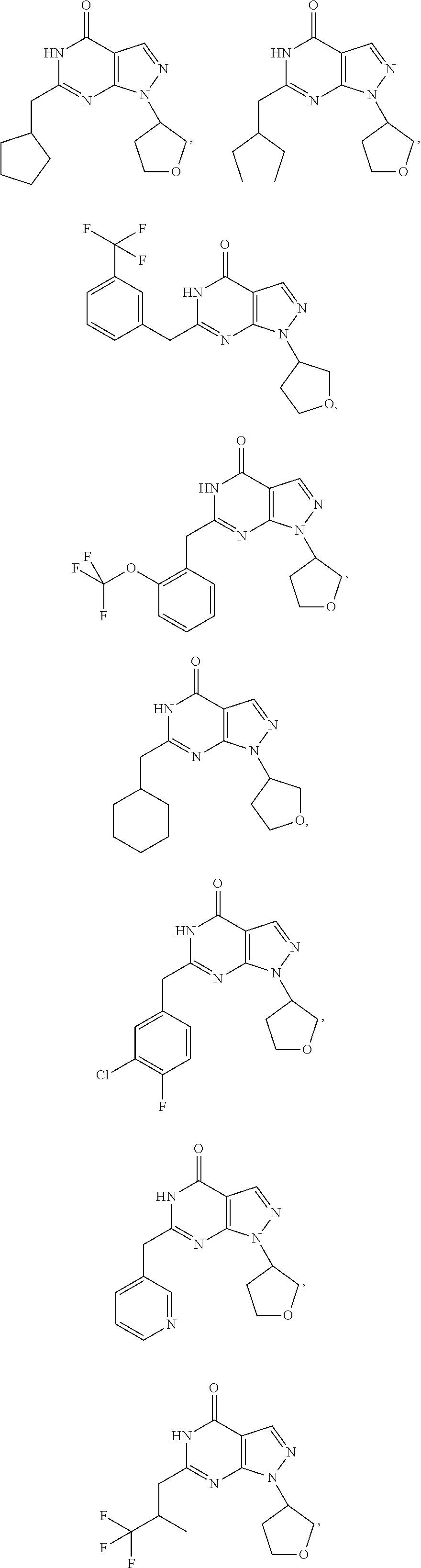

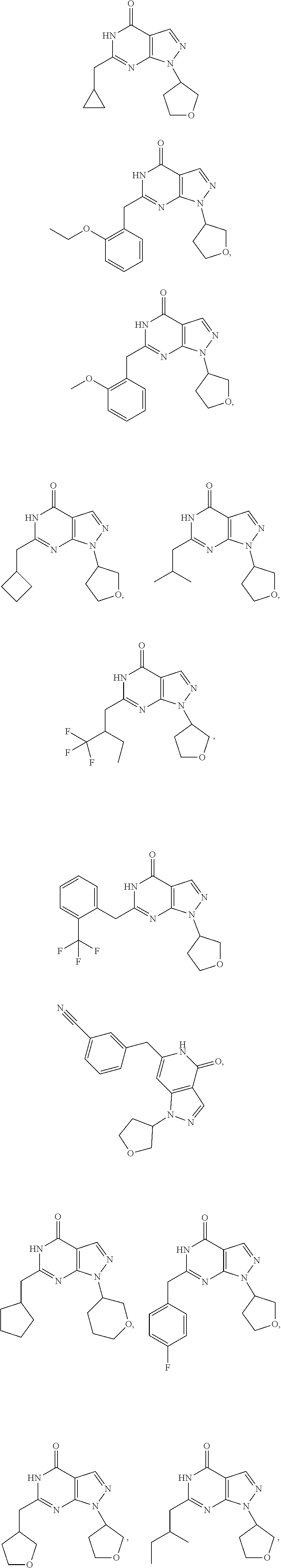

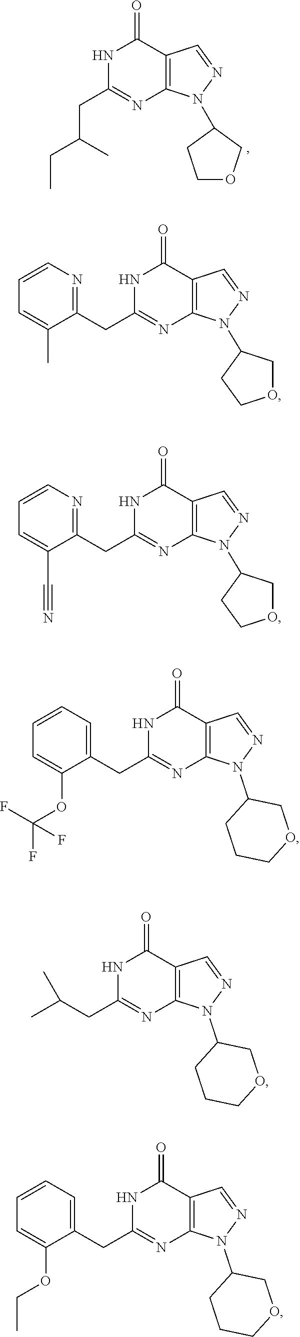 [SMILES: CCC(C)Cc1nc2c(cnn2C2CCOC2)c(=O)[nH]1 ; Cc1cccnc1Cc1nc2c(cnn2C2CCOC2)c(=O)[nH]1 ; N#Cc1cccnc1Cc1nc2c(cnn2C2CCOC2)c(=O)[nH]1 ; O=c1[nH]c(Cc2ccccc2OC(F)(F)F)nc2c1cnn2C1CCCOC1 ; CC(C)Cc1nc2c(cnn2C2CCCOC2)c(=O)[nH]1 ; CCOc1ccccc1Cc1nc2c(cnn2C2CCCOC2)c(=O)[nH]1]

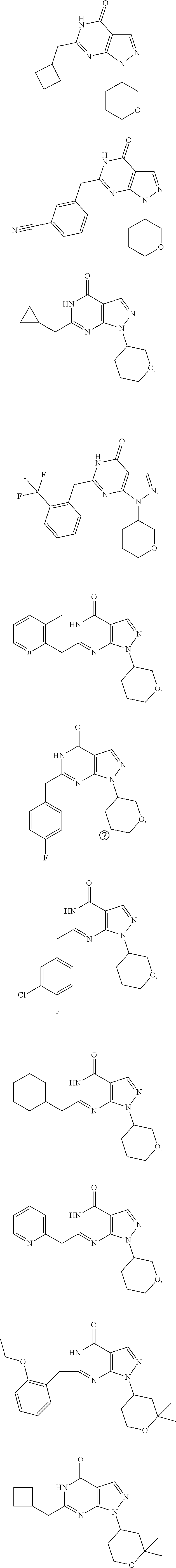

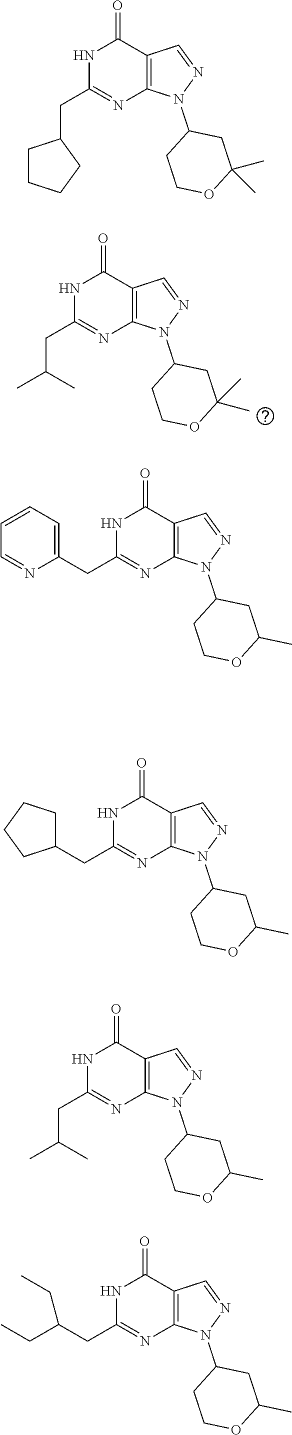

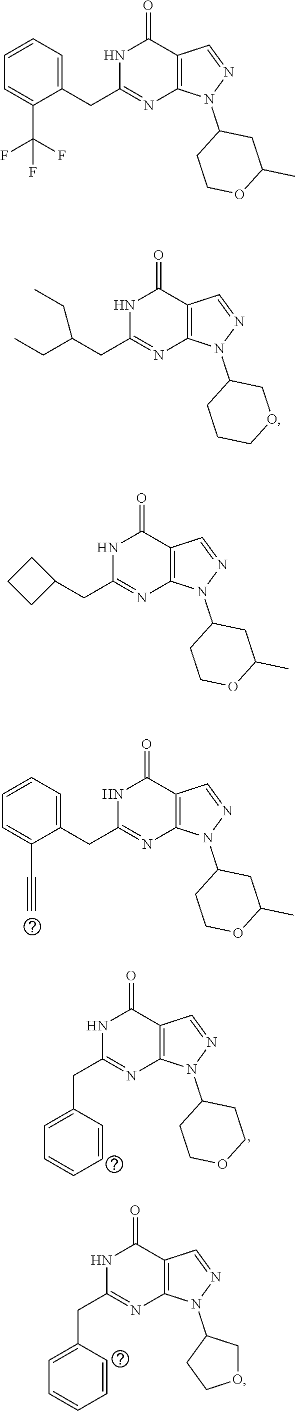

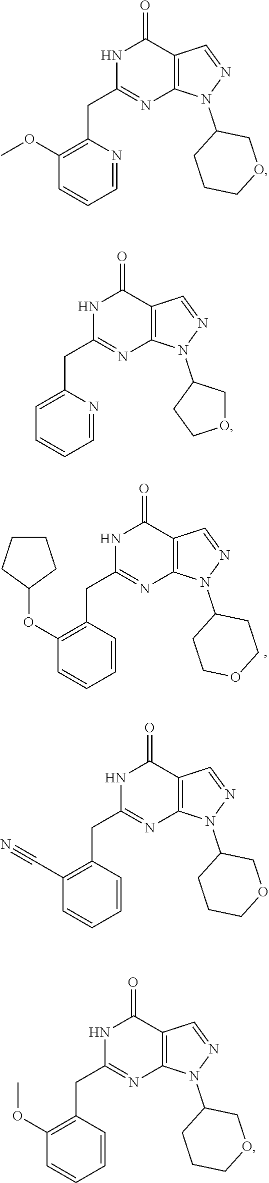 [SMILES: COc1cccnc1Cc1nc2c(cnn2C2CCCOC2)c(=O)[nH]1 ; O=c1[nH]c(Cc2ccccn2)nc2c1cnn2C1CCOC1 ; O=c1[nH]c(Cc2ccccc2OC2CCCC2)nc2c1cnn2C1CCOCC1 ; N#Cc1ccccc1Cc1nc2c(cnn2C2CCCOC2)c(=O)[nH]1 ; COc1ccccc1Cc1nc2c(cnn2C2CCCOC2)c(=O)[nH]1]

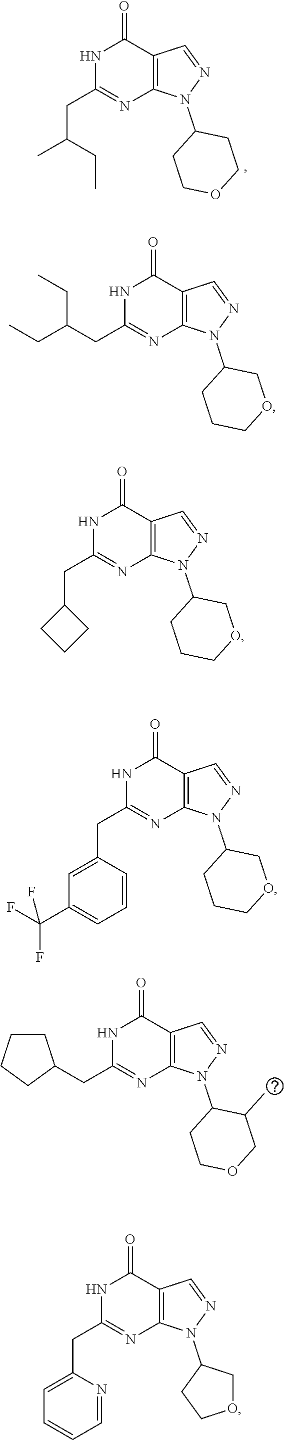 [SMILES: CCC(C)Cc1nc2c(cnn2C2CCOCC2)c(=O)[nH]1 ; CCC(CC)Cc1nc2c(cnn2C2CCCOC2)c(=O)[nH]1 ; O=c1[nH]c(CC2CCC2)nc2c1cnn2C1CCCOC1 ; O=c1[nH]c(Cc2cccc(C(F)(F)F)c2)nc2c1cnn2C1CCCOC1 ; CC1COCCC1n1ncc2c(=O)[nH]c(CC3CCCC3)nc21 ; O=c1[nH]c(Cc2ccccn2)nc2c1cnn2C1CCOC1]

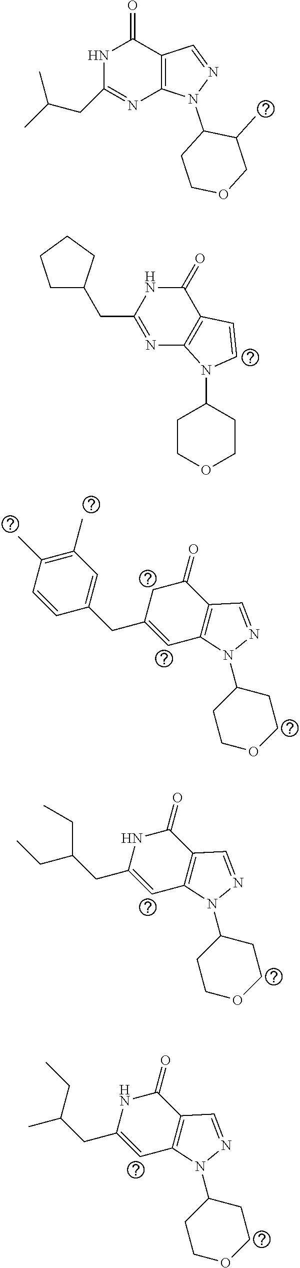

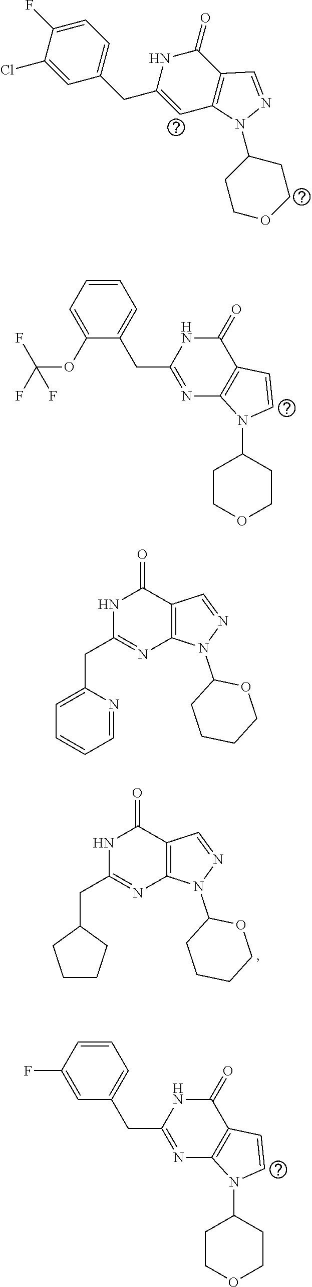 [SMILES: O=c1[nH]c(Cc2ccc(F)c(Cl)c2)cc2c1cnn2C1CCOCC1 ; O=c1[nH]c(Cc2ccccc2OC(F)(F)F)nc2c1ccn2C1CCOCC1 ; O=c1[nH]c(Cc2ccccn2)nc2c1cnn2C1CCCCO1 ; O=c1[nH]c(CC2CCCC2)nc2c1cnn2C1CCCCO1 ; O=c1[nH]c(Cc2cccc(F)c2)nc2c1ccn2C1CCOCC1]

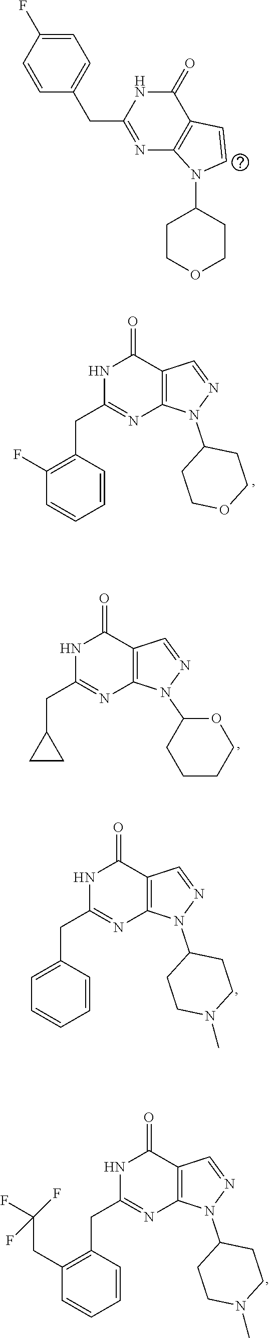 [SMILES: O=c1[nH]c(Cc2ccc(F)cc2)nc2c1ccn2C1CCOCC1 ; O=c1[nH]c(Cc2ccccc2F)nc2c1cnn2C1CCOCC1 ; O=c1[nH]c(CC2CC2)nc2c1cnn2C1CCCCO1 ; CN1CCC(n2ncc3c(=O)[nH]c(Cc4ccccc4)nc32)CC1 ; CN1CCC(n2ncc3c(=O)[nH]c(Cc4ccccc4CC(F)(F)F)nc32)CC1]

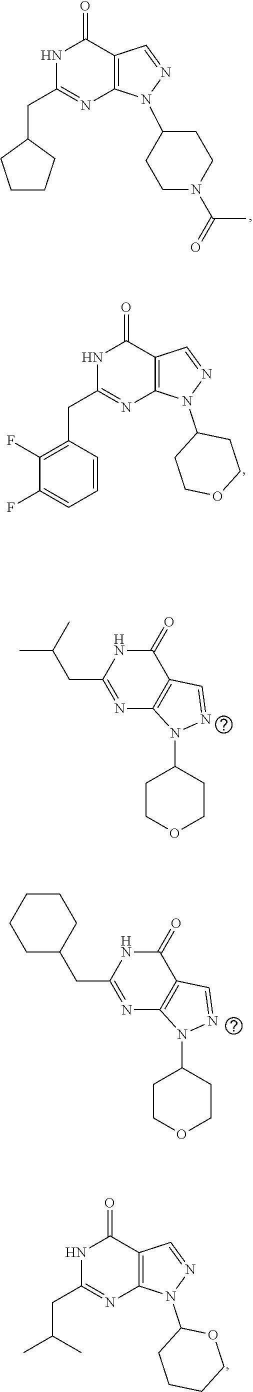

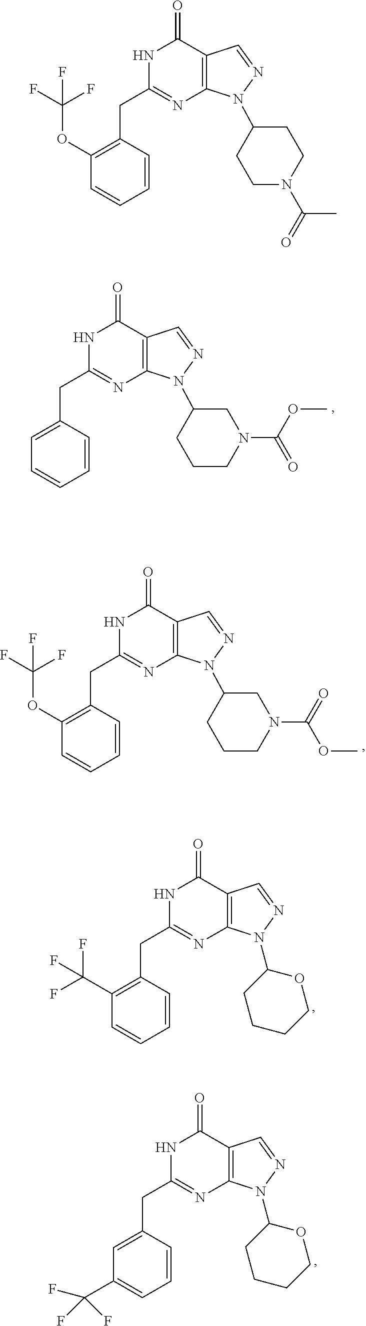 [SMILES: CC(=O)N1CCC(n2ncc3c(=O)[nH]c(Cc4ccccc4OC(F)(F)F)nc32)CC1 ; COC(=O)N1CCCC(n2ncc3c(=O)[nH]c(Cc4ccccc4)nc32)C1 ; COC(=O)N1CCCC(n2ncc3c(=O)[nH]c(Cc4ccccc4OC(F)(F)F)nc32)C1 ; O=c1[nH]c(Cc2ccccc2C(F)(F)F)nc2c1cnn2C1CCCCO1 ; O=c1[nH]c(Cc2cccc(C(F)(F)F)c2)nc2c1cnn2C1CCCCO1]

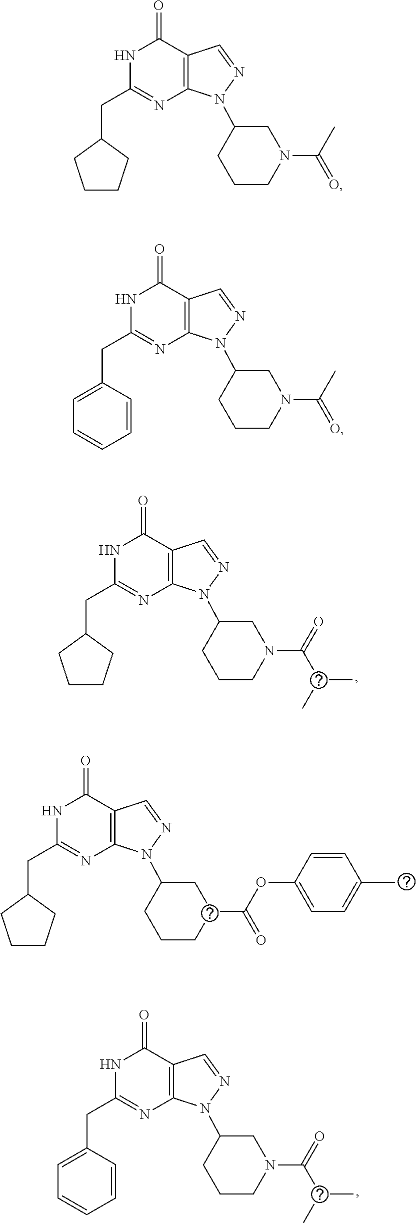 [SMILES: CC(=O)N1CCCC(n2ncc3c(=O)[nH]c(CC4CCCC4)nc32)C1 ; CC(=O)N1CCCC(n2ncc3c(=O)[nH]c(Cc4ccccc4)nc32)C1 ; CC(C)C(=O)N1CCCC(n2ncc3c(=O)[nH]c(CC4CCCC4)nc32)C1 ; Cc1ccc(OC(=O)C2CCCC(n3ncc4c(=O)[nH]c(CC5CCCC5)nc43)C2)cc1 ; CC(C)C(=O)N1CCCC(n2ncc3c(=O)[nH]c(Cc4ccccc4)nc32)C1]

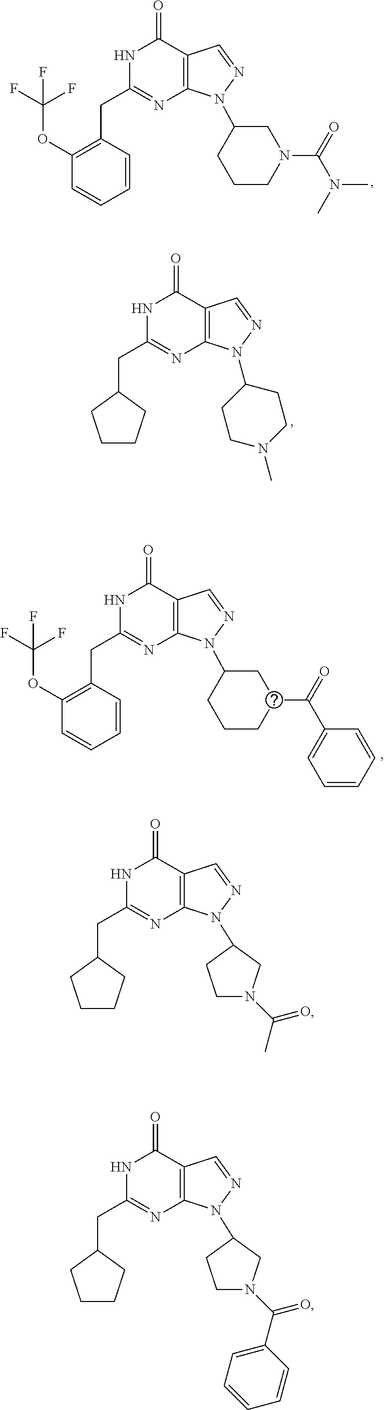 [SMILES: CN(C)C(=O)N1CCCC(n2ncc3c(=O)[nH]c(Cc4ccccc4OC(F)(F)F)nc32)C1 ; CN1CCC(n2ncc3c(=O)[nH]c(CC4CCCC4)nc32)CC1 ; O=C(c1ccccc1)C1CCCC(n2ncc3c(=O)[nH]c(Cc4ccccc4OC(F)(F)F)nc32)C1 ; CC(=O)N1CCC(n2ncc3c(=O)[nH]c(CC4CCCC4)nc32)C1 ; O=C(c1ccccc1)N1CCC(n2ncc3c(=O)[nH]c(CC4CCCC4)nc32)C1]

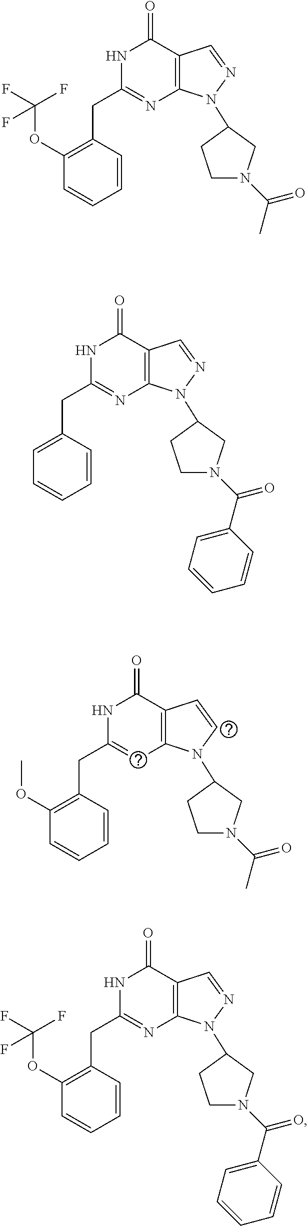 [SMILES: CC(=O)N1CCC(n2ncc3c(=O)[nH]c(Cc4ccccc4OC(F)(F)F)nc32)C1 ; O=C(c1ccccc1)N1CCC(n2ncc3c(=O)[nH]c(Cc4ccccc4)nc32)C1 ; COc1ccccc1Cc1cc2c(ccn2C2CCN(C(C)=O)C2)c(=O)[nH]1 ; O=C(c1ccccc1)N1CCC(n2ncc3c(=O)[nH]c(Cc4ccccc4OC(F)(F)F)nc32)C1]

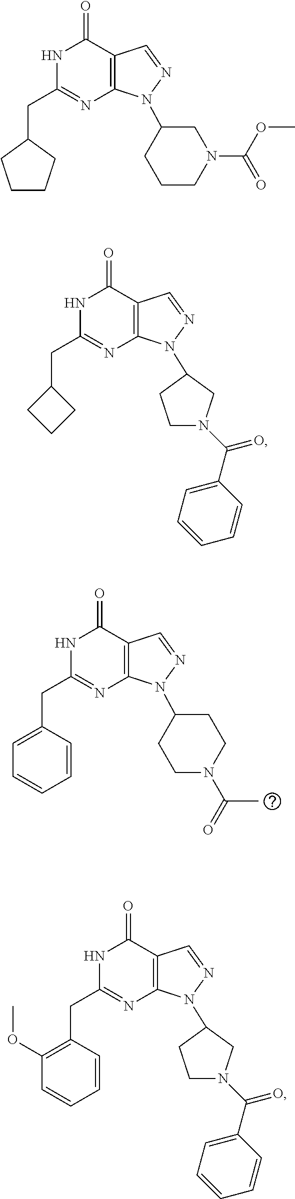 [SMILES: COC(=O)N1CCCC(n2ncc3c(=O)[nH]c(CC4CCCC4)nc32)C1 ; O=C(c1ccccc1)N1CCC(n2ncc3c(=O)[nH]c(CC4CCC4)nc32)C1 ; CC(=O)N1CCC(n2ncc3c(=O)[nH]c(Cc4ccccc4)nc32)CC1 ; COc1ccccc1Cc1nc2c(cnn2C2CCN(C(=O)c3ccccc3)C2)c(=O)[nH]1]

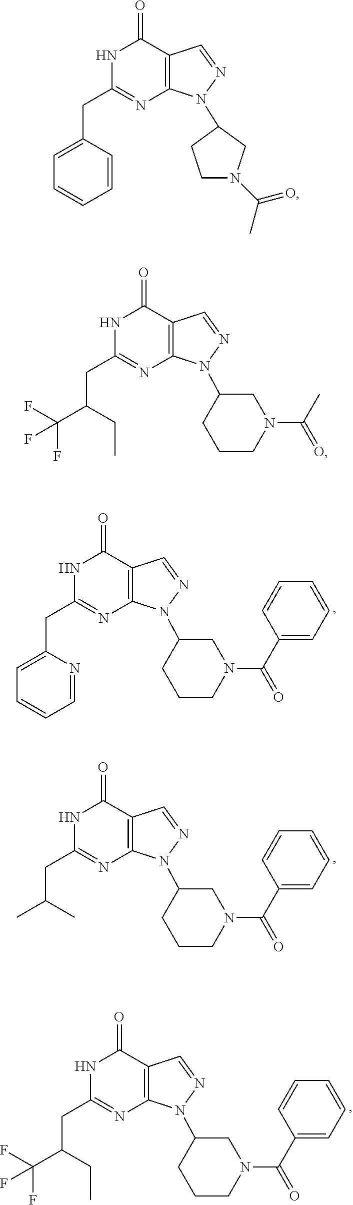 [SMILES: CC(=O)N1CCC(n2ncc3c(=O)[nH]c(Cc4ccccc4)nc32)C1 ; CCC(Cc1nc2c(cnn2C2CCCN(C(C)=O)C2)c(=O)[nH]1)C(F)(F)F ; O=C(c1ccccc1)N1CCCC(n2ncc3c(=O)[nH]c(Cc4ccccn4)nc32)C1 ; CC(C)Cc1nc2c(cnn2C2CCCN(C(=O)c3ccccc3)C2)c(=O)[nH]1 ; CCC(Cc1nc2c(cnn2C2CCCN(C(=O)c3ccccc3)C2)c(=O)[nH]1)C(F)(F)F]

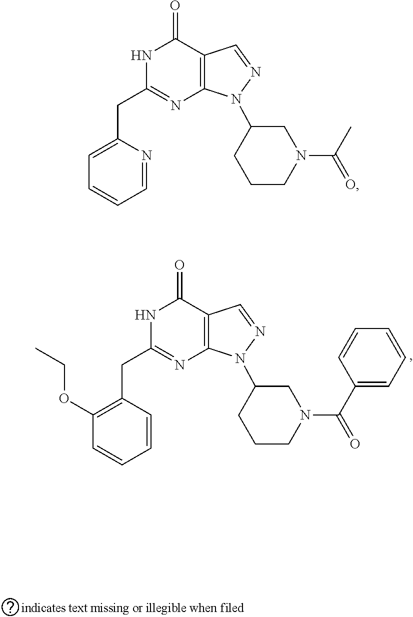 [SMILES: CC(=O)N1CCCC(n2ncc3c(=O)[nH]c(Cc4ccccn4)nc32)C1 ; CCOc1ccccc1Cc1nc2c(cnn2C2CCCN(C(=O)c3ccccc3)C2)c(=O)[nH]1]

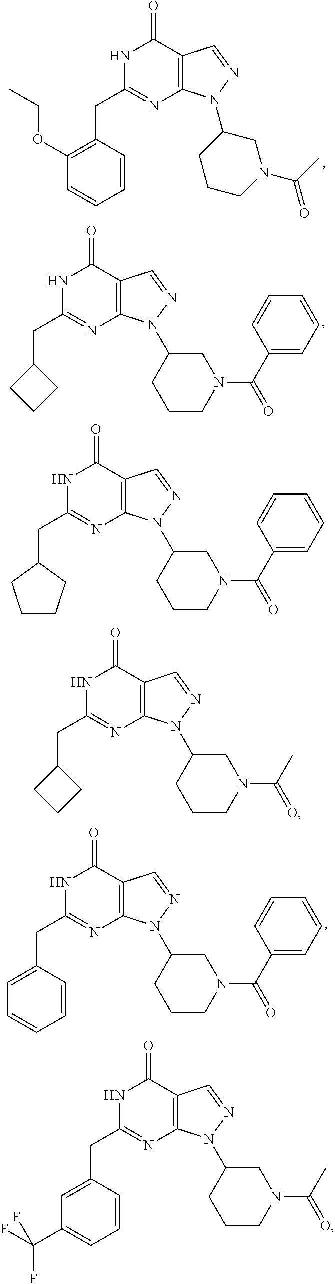

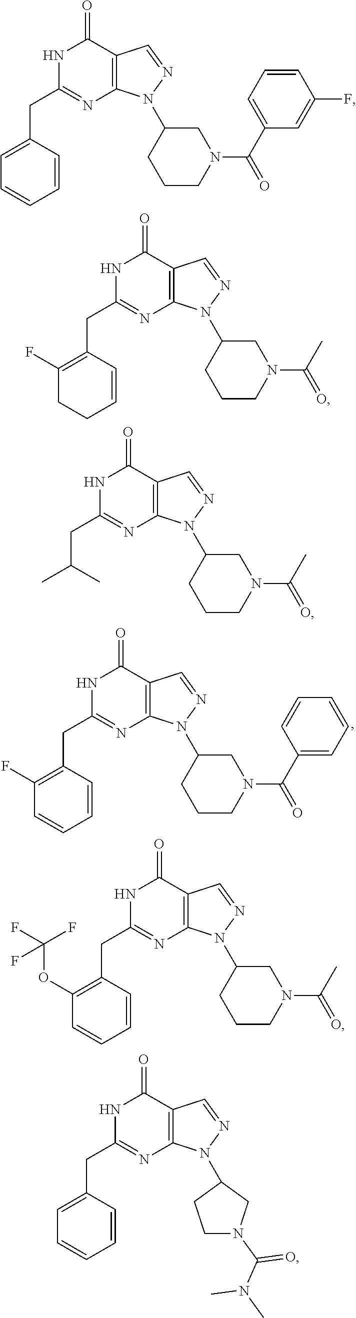

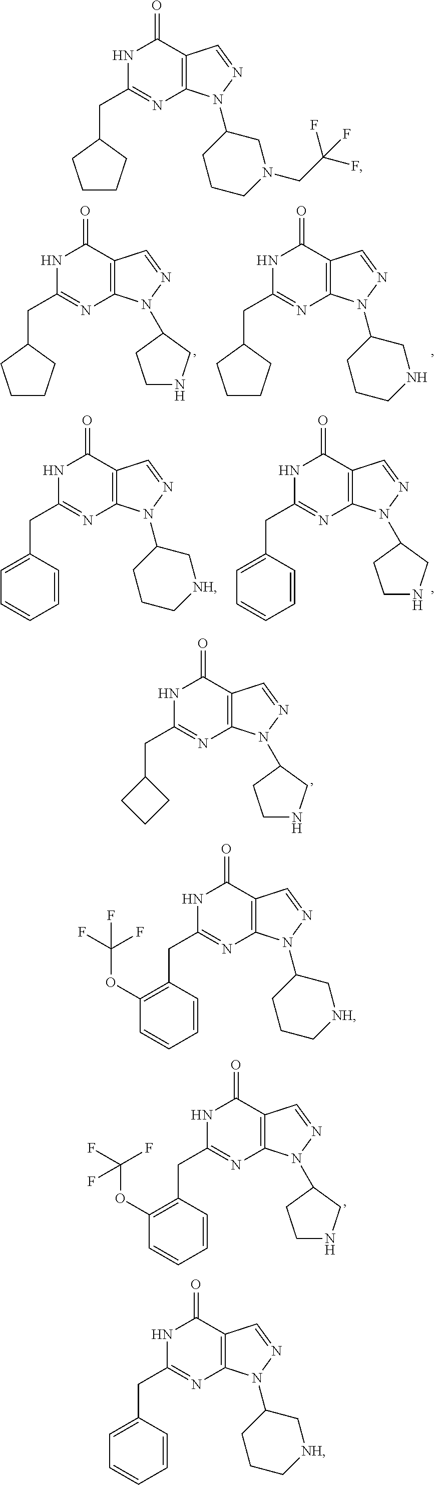

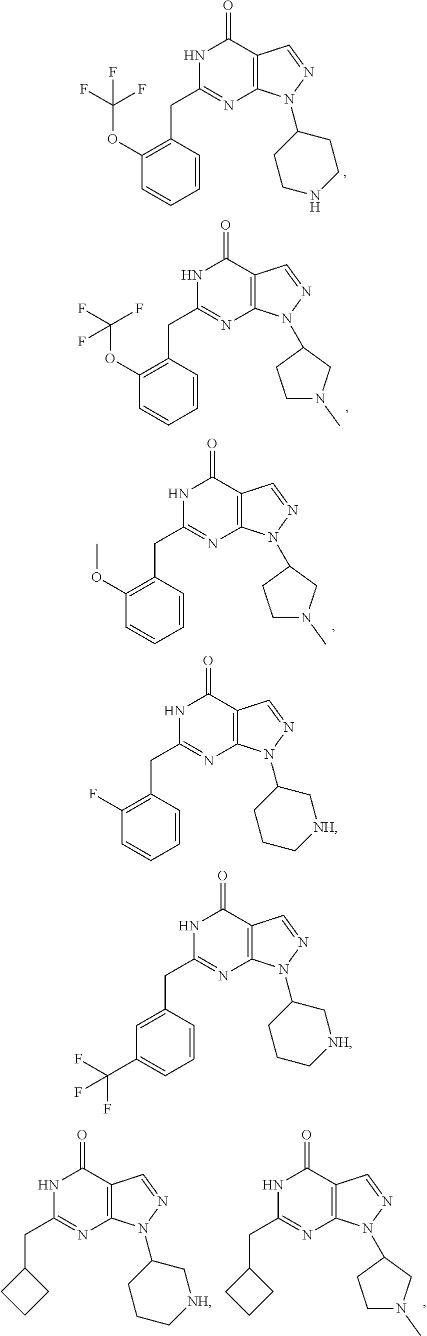

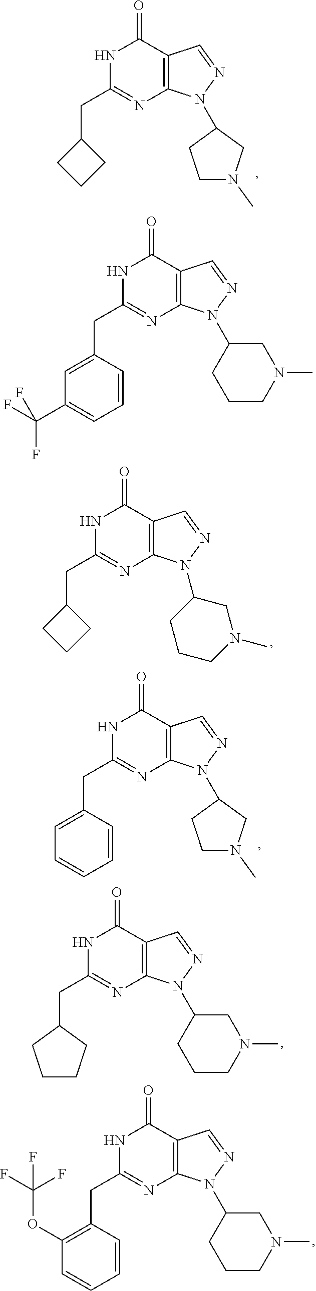

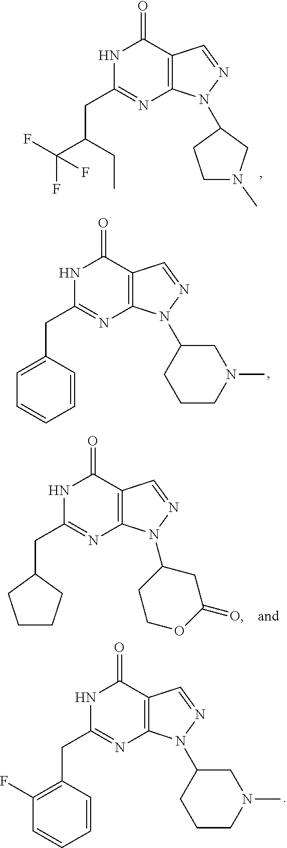

In some embodiments the PDE9 inhibitor is

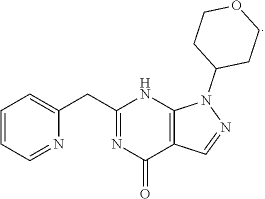 [SMILES: O=c1nc(Cc2ccccn2)[nH]c2c1cnn2C1CCOCC1]

In some cases, as disclosed in U.S. Pat. No. 9,079,905 and WO2010026214, PDE9 inhibitors are represented by a compound of formula (XIX),

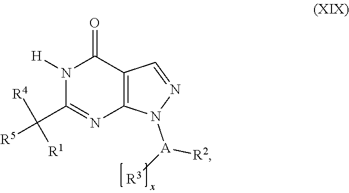

wherein
A is cyclohexyl;
R¹ is selected from the group consisting of phenyl, 2-, 3- and 4-pyridyl, cyclopropyl, cyclobutyl, cyclopentyl, cyclohexyl, ethyl, 1- and 2-propyl, 1- and 2-butyl, 1-, 2- and 3-pentyl, tetrahydrofuranyl and tetrahydropyranyl,
wherein these R¹ groups may optionally be substituted by one or more substituents selected from the group R¹¹ which consists of fluorine, chlorine, bromine, iodine, NC—, C₁₋₆-alkyl-O—, C₁₋₆-alkyl-, CF₃₀—, F₃C—, pyridyl, (R¹⁰)₂N—CO—CH₂—, N-morpholinyl-C₁₋₆-alkyl-, pyrazolyl and phenyl,
and wherein if R¹ is tetrahydrofuranyl or tetrahydropyranyl, it may also be substituted with oxo,
and wherein the pyridyl, pyrazolyl and phenyl group of the aforementioned group R¹ may optionally be substituted with a group selected from fluorine, chlorine, H₃C—, F₃C—CH₃O—, H₂NCO— and NC—;
R² is fluorine;
R³ is fluorine;
R⁴ and R⁵ are independently selected from H and fluorine;
R¹⁰ independently of any other R¹⁰ is selected from H—, C₁₋₆-alkyl-, phenyl and pyridyl; and x is 1;
and salts thereof.

In some cases, as disclosed in U.S. Pat. Nos. 8,912,201 and 9,328,120 and WO2012020022 and WO2012/110440, PDE9 inhibitors are represented by a compound of formula (XX),

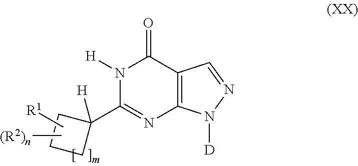

wherein
R¹: is a 5 or 6 membered heteroaryl-group whereby 1, 2, 3 or 4, preferably 1, 2 or 3, of the ring atoms are heteroatoms that are selected independently of each other from N, O or S, whereby said 5 or 6 membered aromatic heteroaryl-group optionally may be substituted by 1, 2, 3 or 4, preferably 1 or 2 substituents, whereby said substituents may be selected independently of one another from the group consisting of fluorine, chlorine, bromine, HO—, NC—, F₃C—, HF₂C—, FH₂C—, methyl, H₂N- and (CH₃)₂N—;
R²: is selected from the group consisting of fluorine, NC—, F₃C—, HF₂C—, FH₂C— and methyl, preferably fluorine, NC—, F₃C— and methyl;
D: is selected from the group consisting of cyclopentyl, cyclohexyl, tetrahydrofuranyl, tetrahydropyranyl, 2-, 3- and 4-pyridyl, whereby cyclopentyl and cyclohexyl optionally may be substituted by 1 or 2 substituents, whereby said substituents may be selected independently of one another from the group consisting of fluorine, NC—, F₃C—, HF₂C— and FH₂C—;
whereby tetrahydrofuranyl and tetrahydropyranyl optionally may be substituted by 1 or 2 substituents, whereby said substituents may be selected independently of one another from the group consisting of fluorine, NC—, F₃C—, FIF₂C— and FH₂C—;
whereby pyridyl optionally may be substituted by 1, 2, 3 or 4 substituents, whereby said substituents may be selected independently of one another from the group consisting of fluorine, chlorine, bromine, NC—, F₃C—, HF₂C—, FH₂C—; F₃C—CH₂—, C₁₋₆-alkyl- and C₃₋₇-cycloalkyl;
m: is selected from 1 or 2, preferably 1;
n: is selected from 0, 1 or 2, preferably, 0 or 1, more preferably 0,
whereby if n=2, these two groups R² are selected independently of one another; and salts, preferably pharmaceutically acceptable salts thereof, solvates thereof and the solvates of the aforementioned salts thereof; with the roviso that the compound is not the following oxadiazolyl-derivative

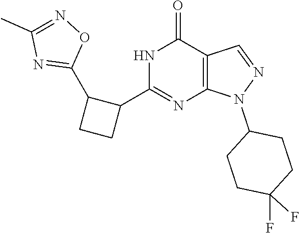 [SMILES: Cc1noc(C2CCC2c2nc3c(cnn3C3CCC(F)(F)CC3)c(=O)[nH]2)n1]

be it in the form of any possible stereoisomer or a mixture of all or some thereof or salt thereof or solvate thereof or a solvate of a salt thereof.

In some cases, as disclosed in U.S. Pat. Nos. 8,623,879 and 9,096,603, PDE9 inhibitors are represented by a compound of formula (XXI),

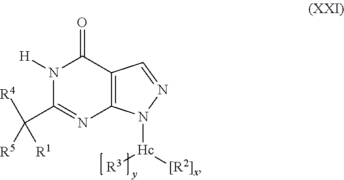

wherein
Hc is selected from the group of tetrahydropyranyl, tetrahydrofuranyl, piperidinyl and pyrrolidinyl;
R¹ is selected from the group of
phenyl, 2-, 3- and 4-pyridyl-, pyrimidinyl, pyrazolyl, thiazolyl, cyclopropyl, cyclobutyl, cyclopentyl, cycloheptyl, cyclopentylmethyl, cyclohexyl, ethyl, 1- and 2-propyl, 1- and 2-butyl-, 1-, 2- and 3-pentyl-, tetrahydrofuranyl and tetrahydropyranyl,
where these R¹ groups may optionally be substituted by one or more substituents independently of one another selected from the group consisting of fluorine, chlorine, bromine, iodine, HO—, oxo, NC—, C₁₋₆-alkyl-O—, C₁₋₆-alkyl-, C₃₋₇-cycloalkyl-O—, C₃₋₇cycloalkyl-C₁₋₃alkyl-O—, CF₃O— and CF₃;
R² independently of any other R² is selected from the group of H—, C₁₋₆-alkyl and oxo, and in cases wherein R² is attached to a nitrogen which is a ring member of Hc, this R² shall be independently of any other R² selected from: H—, C₁₋₆-alkyl-CO—, C₁₋₆-alkyl-O—CO—, C₁₋₆-alkyl-, phenyl-CO—, phenyl-O—CO— and (C₁₋₆-alkyl)₂N—CO—,
where the above-mentioned R² members may optionally be substituted independently of one another by one or more fluorine substituents;
R³ is selected from the group of
H—, hydroxy and C₁₋₆-alkyl-O—, whereby C₁₋₆-alkyl-O— may optionally be substituted by one or more fluorine, chlorine, bromine and HO—;
R⁴ and R⁵ independently of one another are selected from the group of H—, fluorine, and methyl;
x is 0, 1, 2, 3 or 4;
y is 0 or 1;
or a pharmaceutically acceptable salt, a tautomer or a stereoisomer thereof.

In some cases, as disclosed in U.S. Pat. Nos. 8,623,901 and 9,102,679, PDE9 inhibitors are represented by a compound of formula (XXII),

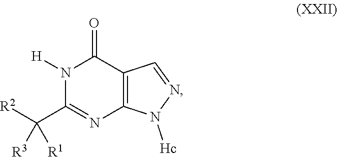

wherein
Hc is tetrahydropyranyl,
wherein one or more carbon ring atom(s) thereof optionally may be substituted by one or by two substituents independently selected from the group of fluorine, NC—, F₃C—, HF₂C—, FH₂C—, F₃C—CH₂—, C₁₋₆-alkyl-, C₁₋₆-alkyl-O— and up to one carbon ring atom may be substituted with oxo;
R¹ is the group, V-W-*
wherein
   W is phenyl;
   V is selected from the group of phenyl or heteroaryl, said heteroaryl being selected from the group of oxadiazolyl, triazolyl, pyrazolyl, pyrrolyl, furanyl, pyridyl, pyrimidyl and pyridazinyl;
   -* is the binding point by which W is attached to the CR²R³ group in formula (I); wherein W and V independently of each other optionally may be substituted by one or more substituents selected from the group of fluorine, chlorine, bromine, C₁₋₆-alkyl-, F₃C—, HF₂C—, FH₂C—, F₃C—CH₂—, F₃C—O—, HF₂C—O—, C₃₋₇-heterocycloalkyl-, H—O—C₁₋₆-alkyl-, C₁₋₆-alkyl-O—C₁₋₆-alkyl-, C₃₋₇-cycloalkyl-O—C₁₋₆-alkyl-, C₃₋₇-cycloalkyl-C₁₋₃-alkyl-O—C₁₋₆-alkyl-, phenyl-O—C₁₋₆-alkyl-, benzyl-O—C₁₋₆-alkyl-, H—O—, C₁₋₆-alkyl-O—, C₃₋₇-cycloalkyl-O—, C₃₋₇-cycloalkyl-C₁₋₃-alkyl-O—, phenyl-O—, benzyl-O—, N-morpholinyl, and NC—;
R² is selected from the group of H—, fluorine, F₃C—, HF₂C—, FH₂C—, and C₁₋₃-alkyl;
R³ is selected from the group of H—, fluorine, F₃C—, HF₂C—, FH₂C—, and C₁₋₃-alkyl.

In some cases, as disclosed in PCT International Publication WO2019204298 and U.S. patent Ser. No. 10/822,346, PDE9 inhibitors are represented by a compound of formula (XXIII),

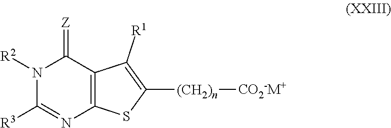

or stereoisomers, tautomers or hydrates thereof,
wherein:
R¹ is hydrogen, (C₁-C₆)alkyl, (C₁-C₆)alkoxy or (C₁-C₆) haloalkyl containing 1-6 halogen atoms;
R² is hydrogen, (C₁-C₆)alkyl, [(C₁-C₆)alkylene]aryl or amino;
R³ is hydrogen, (C₁-C₆)alkyl, (C₁-C₆)alkenyl, [(C₁-C₆)alkylene]N(R⁴)(R⁵), [(C₁-C₆)alkylene]S(R⁴) or —X—Y, or R² and R³ together with the atoms to which they are attached form a heterocyclyl;
   R⁴ and R⁵ are independently H or (C₁-C₆)alkyl;
X is a chemical bond, —CH₂—CH(OH)—, —CH(C₆H₅)—, —CO— —CH₂CH₂—CH₂CO—, —COCH2-,
S, O or NH;
Y is cycloalkyl, heterocyclyl, aryl or heteroaryl;
Z is S or O;
N is 0, 1, 2, 3 or 4;
M⁺ is

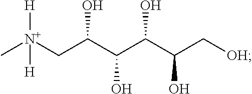

and
wherein any alkyl, alkylene, cycloalkyl, heterocyclyl, heteroaryl or aryl is optionally substituted with 1, 2 or 3 groups selected from OH, CN, halogen, (C₁-C₆)alkyl, O(C₁-C₆)alkyl, (C₂-C₆)alkenyl, haloalkyl, amino, oxo and nitro.

In some cases, as disclosed in WO2018009899, PDE9 inhibitors are represented by a compound of formula (XXIV),

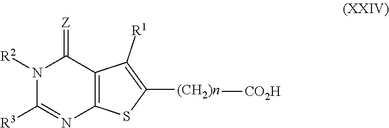

wherein:
R¹ is hydrogen, C₁₋₆ alkyl, C₁₋₆ alkoxyC₁₋₆ alkyl or C₁₋₆ haloalkyl containing 1-6 halogen atoms,
R² is hydrogen, C₁₋₆ alkyl, phenylC₁₋₆ alkyl or amino,
R³ is C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X group, or R² and R³ may together form tetramethylene,
X is a chemical bond, or CH₂, CH(OH), CH(C₆H₅), CO, CH₂CH₂, CH₂CO, COCH₂, S, O or NH and
Y is an aromatic carbocyclic group, benzyl, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from halogen atom, C₁₋₆ alkyl, C₁₋₆ haloalkyl containing 1-6 halogen atoms, C₁₋₆ haloalkyloxy containing 1-6 halogen atoms, C₁₋₆ haloalkylthio containing 1-6 halogen atoms, C₁₋₆ alkoxy, C₁₋₆ alkylthio, C₁₋₄ alkylenedioxy, carboxyl, C₁₋₆ alkoxycarbonyl, oxo, amino, nitro and phenyl,
Z is S or 0, and n is 0 or an integer of 1-4; and pharmaceutically acceptable salts thereof.

In some cases, as disclosed in WO2006135080, U.S. Pat. Nos. 8,293,754, and 8,377,944, PDE9 inhibitors are represented by a compound of formula (XXV),

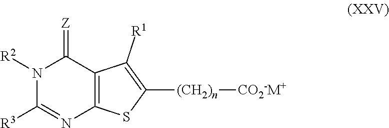

in which
   R¹ is hydrogen, C₁₋₆ alkyl, C₁₋₆ alkoxyC₁₋₆ alkyl or C₁₋₆ haloalkyl containing 1-6 halogen atoms,
   R² is hydrogen, C₁₋₆ alkyl, phenylC₁₋₆ alkyl or amino,
   R³ is C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X— group, or
   R² and R³ may together form tetramethylene,
   X is a direct bond, or CH₂, CH(OH), CH(C₆H₅), CO, CH₂CH₂, CH₂CO, COCH₂, S, O or NH and
   Y is an aromatic carbocyclic group, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from the group consisting of halogen atom, C₁₋₆ alkyl, C₁₋₆ haloalkyl containing 1-6 halogen atoms, C₁₋₆ haloalkyloxy containing 1-6 halogen atoms, C₁₋₆ haloalkylthio containing 1-6 halogen atoms, C₁₋₆ alkoxy, C₁₋₆ alkylthio, C₁₋₄ alkylenedioxy, carboxyl, C₁₋₆ alkoxycarbonyl, oxo, amino, nitro and phenyl,
   Z is S or O, and
   n is 0 or an integer of 1-4,
   with the proviso that a case wherein R¹ is methyl, R² is hydrogen, R³ is benzyl, Z is O and n is 0 is excluded,
or a salt thereof.

In some embodiments, the PDE9 inhibitor selected from the group consisting of a compound having a structure of:

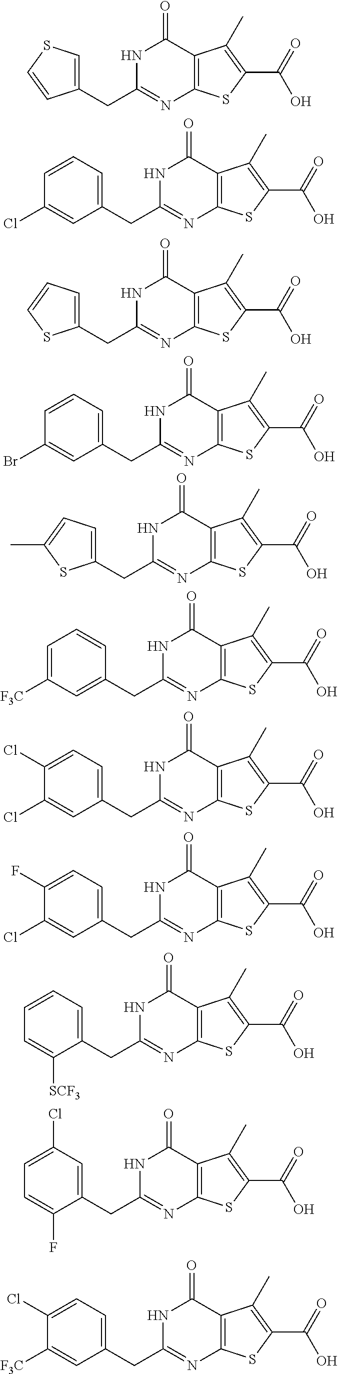

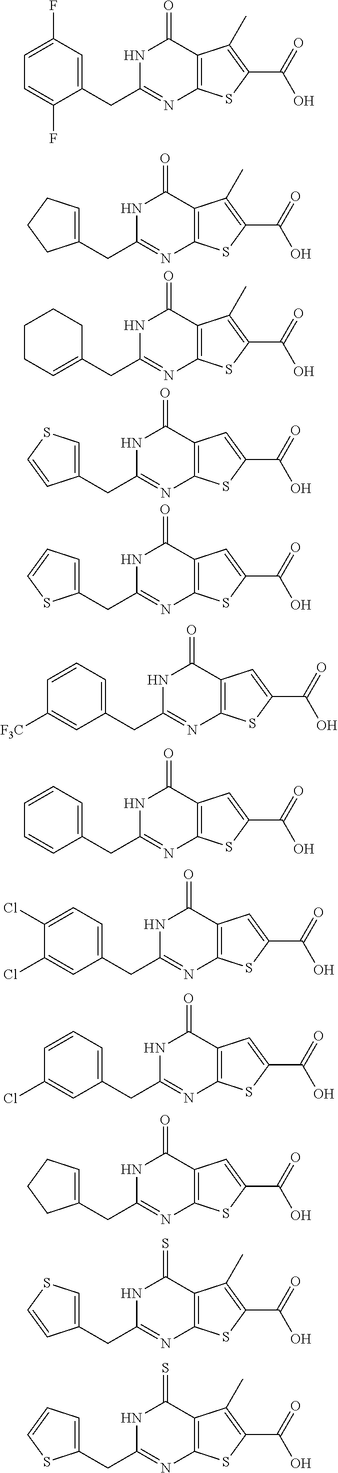

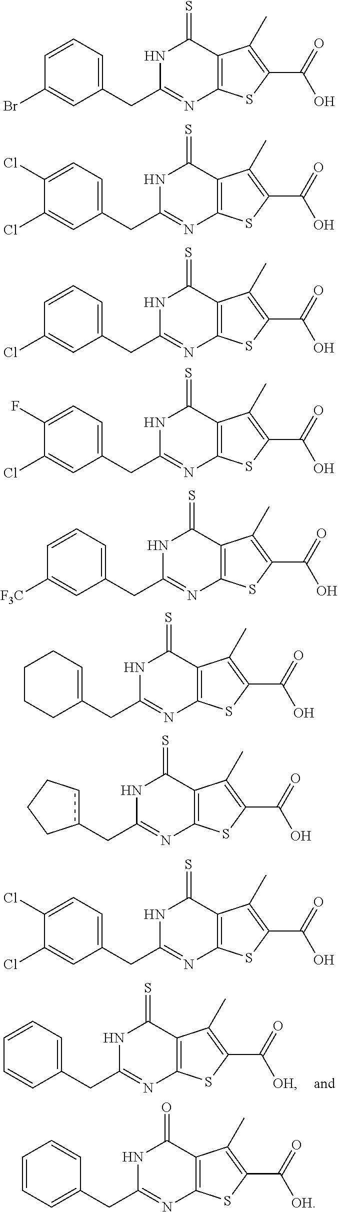

In some embodiments the PDE9 inhibitor is

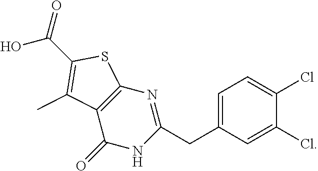 [SMILES: Cc1c(C(=O)O)sc2nc(Cc3ccc(Cl)c(Cl)c3)[nH]c(=O)c12]

In some cases, as disclosed in WO2012004900, PDE9 inhibitors are represented by a compound of formula (XXVI),

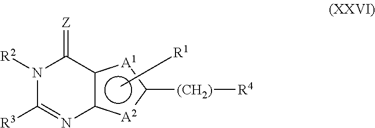

in which
R¹ stands for hydrogen, C₁₋₆ alkyl, C₁₋₆ alkoxy C₁₋₆ alkyl or C₁₋₆ haloalkyl containing 1-6 halogen atoms,
R² stands for hydrogen, C₁₋₆ alkyl, phenyl C₁₋₆ alkyl or amino,
R³ stands for C₁₋₆ alkyl, C₂₋₆ alkenyl, carbamoyl C₁₋₆ alkyl, amino C₁₋₆ alkyl, C₁₋₆ alkylamino C₁₋₆ alkyl, di-(C₁₋₆ alkyl)aminoC₁₋₆ alkyl, C₁₋₆ alkylthio or Y—X— group, or
R² and R³ may together form tetramethylene,
R⁴ stands for carboxylic acid or its equivalent,
X standing for a direct bond, or CH₂, CH(OH), CH(C₆H₅), CO, CH₂CH₂, CH₂CO, COCH₂, S, O or NH,
Y standing for aromatic carbocyclic group, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from halogen atom,
C₁₋₆ alkyl, C₁₋₆ haloalkyl containing 1-6 halogen atoms, C₁₋₆ haloalkyloxy containing 1-6 halogen atoms, C₁₋₆ haloalkylthio containing 1-6 halogen atoms, C₁₋₆ alkoxy, C₁₋₆ alkylthio, C₁₋₄alkylenedioxy, carboxyl, C₁₋₆ alkoxycarbonyl, oxo, amino, nitro and phenyl,
Z stands for S or O,
n is 0 or an integer of 1-4, and
one of A¹ and A² stands for carbon atom and the other stands for sulfur atom,
R¹ is attached to the carbon atom represented by either A¹ or A²,
with the proviso that there is excluded a case wherein A¹ is carbon atom, A² is sulfur atom and R⁴ is hydroxy 1,
or salts of the compounds are provided.

In some cases, as disclosed in JPWO2008072778 and US20100113484, PDE9 inhibitors are represented by a compound of the following formulae (XXVII)—(XXXII) can be named:
thienopyrimidine derivatives represented by Formula (XXVII),

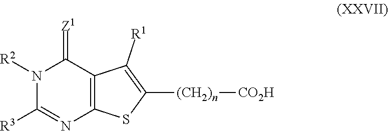

in the formula,
   R¹ stands for hydrogen, C₁₋₆ alkyl, C₁₋₆ alkoxyC₁₋₆ alkyl, or C₁₋₆ haloalkyl having 1-9 halogen atoms,
   R² stands for hydrogen, C₁₋₆ alkyl, phenylC₁₋₆ alkyl or amino,
   R³ either stands for C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X—, or
   R² and R³ may together form tetramethylene,
   X stands for a direct bond or CH₂, CH(OH), CH(C₆H₅), CO, CH₂CH₂, CH₂CO, COCH₂, S, O, or NH,
   Y stands for an aromatic carbocyclic group, aromatic heterocyclic group, C₄₋₇ cycloalkyl, C₄₋₇ cycloalkenyl, 5- to 7-membered saturated heterocyclic group containing 1 or 2 nitrogen atoms, or 5- to 7-membered saturated heterocyclic group forming a condensed ring with a 5- to 6-membered saturated cyclic group and containing 1 or 2 nitrogen atoms, each of which may be optionally substituted with 1-3 substituents selected from halogen, C₁₋₆ alkyl, C₁₋₆ haloalkyl having 1-9 halogen atoms, C₁₋₆ alkoxy, C₁₋₆ haloalkoxy having 1-9 halogen atoms, C₁₋₆ alkylthio, C₁₋₆haloalkylthio having 1-9 halogen atoms,
   C₁₋₄ alkylenedioxy, carboxy, C₁₋₆ alkoxycarbonyl, oxo, amino, nitro and phenyl,
   Z¹ stands for S or 0, and
   n is 0 or an integer of 1-4,
or salts thereof;
   quinazoline derivatives represented by Formula (XXVIII),

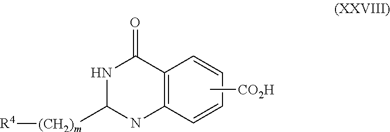

in the formula,
   R⁴ stands for phenyl or aromatic heterocyclic group which may be optionally substituted with 1-3 substituents selected from halogen, C₁₋₆ alkyl, C₁₋₆ haloalkyl having 1-9 halogen atoms and C₁₋₆ alkoxy, and
   m is an integer of 1-3,
or salts thereof;
   quinoxaline derivatives represented by Formula (XXIX),

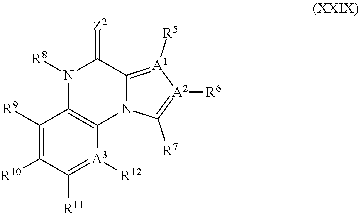

in the formula,
   R⁵ and R⁶ each independently stands for hydrogen; halogen; C₁₋₆ alkyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group; acyl which is optionally substituted with hydroxy, C₁₋₆ alkyl, C₂₋₆ alkenyl, C₂₋₆ alkynyl, C₁₋₆ haloalkyl having 1-9 halogen atoms, C₁₋₆ alkoxy, C₁₋₆ haloalkoxy having 1-9 halogen atoms, amino, carbocyclic group or heterocyclic group; amino which is optionally substituted with 1-2 substituents selected from C₁₋₆ alkyl, C₂₋₆ alkenyl, C₂₋₆ alkynyl, alkanoyl, carbocyclic group and heterocyclic group; hydroxy; or pyrimidinyl which is optionally substituted with halogen, C₁₋₆ alkyl, C₁₋₆ alkoxy, C₁₋₆ haloalkoxy having 1-9 halogen atoms, nitro or amino,
   R⁷ stands for C₁₋₉ alkyl, C₂₋₉ alkenyl or C₂₋₉ alkynyl which are optionally substituted with hydroxy, halogen, C₁₋₆ alkoxy, C₁₋₆ haloalkoxy having 1-9 halogen atoms, carboxy,
   C₁₋₆ alkoxycarbonyl, alkanoyl, amino (here the amino group may further be substituted with 1-2 substituents selected from C₁₋₆ alkyl, C₂₋₆ alkenyl, C₂₋₆ alkynyl, C₁₋₆haloalkyl having 1-9 halogen atoms, alkanoyl, carbocyclic group and heterocyclic group), amido, carbamoyl, oxo, carbocyclic or heterocyclic group (here the carbocyclic group and heterocyclic group each may further be substituted with hydroxy, halogen, C₁₋₆ alkyl,
   C₂₋₆ alkenyl, C₂₋₆ alkynyl, C₁₋₆ alkoxy, carboxy, C₁₋₆ alkoxycarbonyl, amino, amido or carbamoyl); aryl, saturated carbocyclic group or saturated heterocyclic group, each of which is optionally substituted with halogen, hydroxy, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with halogen, hydroxy, C₁₋₆ alkoxy, C₁₋₆ haloalkoxy having 1-9 halogen atoms, carboxy, C₁₋₆ alkoxycarbonyl, alkanoyl, amino, amido, carbamoyl, carbocyclic group or heterocyclic group, independently of each other), C₁₋₆haloalkoxy having 1-9 halogen atoms, carboxy, C₁₋₆ alkoxycarbonyl, alkanoyl, amino, amido, carbamoyl, carbocyclic group or heterocyclic group; carboxy; C₁₋₆ alkoxycarbonyl (here the C₁₋₆ alkoxy moiety in the C₁₋₆ alkoxycarbonyl may further be substituted with hydroxy, halogen, C₁₋₆ alkoxy, C₁₋₆haloalkoxy having 1-9 halogen atoms, carboxy,
   C₁₋₆ alkoxycarbonyl, amino, amido, carbamoyl, carbocyclic group or heterocyclic group); amido (here the amino moiety in the amido may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group); or carbamoyl (here the amino moiety in the carbamoyl may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), R⁸ stands for hydrogen; hydroxy; C₁₋₆ alkyl which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; or amino which is optionally substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group,
   R⁹ and R¹² each independently stands for hydrogen; halogen; C₁₋₆ alkyl, C₂₋₆ alkenyl or
   C₁₋₆ alkoxy each of which is optionally substituted with hydroxy, halogen, C₁₋₆ alkoxy,
   C₁₋₆haloalkoxy having 1-9 halogen atoms, carboxy, C₁₋₆ alkoxycarbonyl, alkanoyl, amino, amido, carbamoyl or oxo; cyano; or nitro,
   R¹⁰ and R¹¹ each independently stands for hydrogen; halogen; C₁₋₆ alkyl, C₂₋₆ alkenyl, C₂₋₆ alkynyl or C₁₋₆ alkoxy each of which is optionally substituted with hydroxy, halogen,
   C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); cyano; amino which is optionally substituted with 1-2 substituents selected from C₁₋₆ alkyl, C₂₋₆ alkenyl,
   C₂₋₆ alkynyl (here the C₁₋₆ alkyl, C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group and heterocyclic group), alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); carbocyclic group or heterocyclic group each of which is optionally substituted with hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆haloalkoxy having 1-9 halogen atoms, carboxy,
   C₁₋₆ alkoxycarbonyl, C₁₋₆ alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, amino, amido or carbamoyl; COR¹³; or SO₂R¹³
   R¹³ stands for hydrogen; hydroxy; C₁₋₆ alkyl, C₂₋₆ alkenyl, C₂₋₆ alkynyl or C₁₋₆ alkoxy, each of which is optionally substituted with hydroxy, halogen, C₁₋₆ alkoxy, C₁₋₆haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy,
   C₁₋₆ alkoxycarbonyl, amino, amido or carbamoyl); amino which may be substituted with 1-2 substituents selected from C₁₋₆ alkyl, C₂₋₆ alkenyl, C₂₋₆ alkynyl, (here the C₁₋₆ alkyl,
   C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy,
   C₁₋₆ alkoxycarbonyl, alkanoyl, amino, amido, carbamoyl, oxo, carbocyclic group or heterocyclic group), C₁₋₆haloalkyl having 1-9 halogen atoms, alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); or aziridin-1-yl, azetidin-1-yl, pyrrolidin-1-yl, piperidin-1-yl, piperazin-1-yl, morpholin-1-yl or pyrazol-1-yl, each of which may be substituted with 1-2 substituents selected from hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆haloalkoxy having 1-9 halogen atoms, carboxy,
   C₁₋₆ alkoxycarbonyl, alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group and heterocyclic group (here the carbocyclic group and heterocyclic group each may further be substituted with hydroxy, halogen, C₁₋₆ alkyl,
   C₂₋₆ alkenyl, C₂₋₆ alkynyl, C₁₋₆ alkoxy, carboxy, C₁₋₆ alkoxycarbonyl, amino, amido or carbamoyl),
   Z² stands for S or O,
   A¹, A² and A³ stand for N or C, independently of each other, with the proviso that R⁵, R⁶ and R¹² are respectively absent where A¹, A² and A³ respectively stand for N, or salts thereof;
   pyrazolopyrimidine derivatives represented by Formula (XXX),

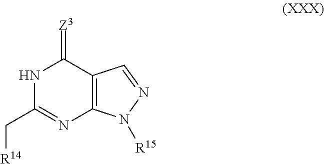

in the formula,
   R¹⁴ stands for phenyl which is substituted with 1-5 substituents selected from halogen, C₁₋₆ alkyl, trifluoromethyl, trifluoromethoxy, cyano, hydroxy, nitro and C₁₋₆ alkoxy,
   R¹⁵ stands for pentan-3-yl or C₄₋₆ cycloalkyl, and
   Z³ stands for S or O,
or salts thereof; and
   pyrazolopyrimidine derivatives represented by Formula (XXXI)

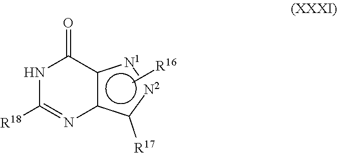

in the formula,
   R¹⁶ stands for hydrogen or C₁₋₆ alkyl, here the R¹⁶ binding to N¹ or N²,
   R¹⁷ stands for C₁₋₆ alkyl which is optionally substituted with hydroxy or alkoxy; C₃₋₇ cycloalkyl which is optionally substituted with alkyl, hydroxy or alkoxy; saturated 5- to 6-membered heterocyclic ring which is optionally substituted with alkyl, hydroxy or alkoxy; het¹; or Ar¹,
   R¹⁸ stands for C₁₋₆ alkyl which is optionally substituted with 1-2 substituents selected from optionally Ar²-substituted or C₁₋₆ alkyl-substituted C₃₋₇ cycloalkyl, OAr², SAr², NHC(O)C₁₋₆ alkyl, het², xanthine and naphthalene,
   here Ar¹ and Ar² standing for the group represented by the following formula (XXXII), independently of each other,

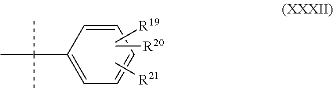

[in the formula, R¹⁹, R²⁰ and R²¹ are either selected from hydrogen, halogen, phenoxy, phenyl, CF₃, OCF₃, R²², SR²² and OR²² (here R²² standing for het³ or C₁₋₆ alkyl which is optionally substituted with phenyl, which phenyl being optionally further substituted with 1-3 substituents selected from halogen, CF₃, OCF₃, C₁₋₆ alkyl and C₁₋₆ alkoxy), or R¹⁹ and R²⁰ together forming 3- or 4-atomic linker optionally containing 1-2 hetero atoms selected from O, S and N],
here het¹, het² and het³ may be the same or different, standing for aromatic 5- to 6-membered heterocyclic ring containing 1-3 hetero atoms selected from O, S and N, the heterocyclic ring being optionally substituted with 1-3 substituents selected from C₁₋₆ alkyl, C₁₋₆ alkoxy, halogen, and phenyl which may further be substituted with 1-3 substituents selected from halogen and C₁₋₆ alkyl, or salts thereof.

In some cases, as disclosed in WO2004096811, PDE9 inhibitors are represented by a compound of formula (XXXIII),

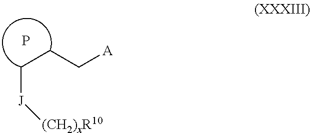

a stereoisomer or prodrug thereof, or a pharmaceutically acceptable salt of said compound, stereoisomer, or prodrug, wherein: A is

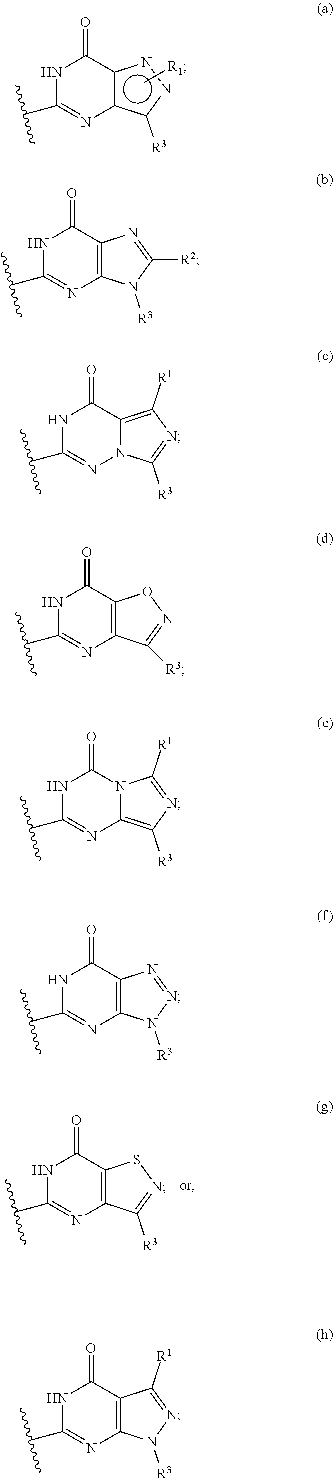

P, including the carbon atoms to which it is attached, is (C₃-C₈)cycloalkyl, (C₃-C₈)heterocycloalkyl, aryl, or heteroaryl; optionally and independently substituted with from 1 to 3 substituents independently selected from halogen, (C₁-C₅)alkyl, (C C₅)alkoxy, and trifluoromethyl;
J is O, S, —N(R¹⁵)—, —N(R¹⁵)CO—, —CON(R¹⁵)—, —SO₂ N(R¹⁵)—, or —N(R¹⁵) SO₂—; x is 0, 1, 2, 3, 4, 5, or 6;
R¹⁰ is —CO₂H, —CONR³⁰R³¹, —NR³⁰R³¹, or —N(R¹⁵)SO₂R⁴⁰;
R and R² are independently H or (CrC₃)alkyl; R³ is (CrC₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C C₅)alkyl, and (C C₅)alkoxy; R⁵ is H or (C₁-C₅)alkyl;
R³⁰ and R³¹ are taken separately and are independently H, (C_{ι}-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C C₅)alkyl, —CO₂R⁴⁰, —COR⁴⁰, —OR⁴⁰, —CONR⁵⁰R⁵¹, —NR⁵⁰R⁵¹, and —SO₂R⁴⁰; or
R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C C₅)alkyl, —CO₂R⁴⁰, —COR⁴⁰, —OR⁴⁰, —CONR⁵⁰R⁵¹, —NR⁵⁰R⁵¹, and —SO₂R⁴⁰;
R⁴⁰ is H, (C C₅)alkyl, (C₃-C₈)cycloalkyl, (C₃-C₈)heterocycloalkyl, aryl, or heteroaryl;
R⁵⁰ and R⁵¹ are taken separately and are independently H, (C-ι-C₅)alkyl, (C₃-C₈)cycloalkyl, (C₃-C₈)heterocycloalkyl, aryl, or heteroaryl; or
R⁵⁰ and R⁵¹ are taken together with the nitrogen atom to which they are attached to form a 5- to 8-membered heterocycloalkyl ring, said ring optionally having 1 additional heteroatom independently selected from N, O, and S.

In some cases, as disclosed in U.S. Pat. No. 8,901,126 and WO2012/033101, PDE9 inhibitors are represented by a compound of formula (XXXIV) or a salt thereof,

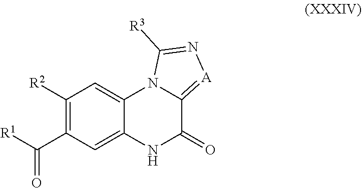

wherein
A is N or CH,
R¹ is 2,3-dihydroindolyl, 1,3-dihydroisoindolyl or dihydropyrrolopyridyl, each of which may be substituted,
R² is halogen, or lower alkyl, —O-lower alkyl or cycloalkyl, each of which may be substituted,
R³ is lower alkyl, cycloalkyl or a saturated heteroring, each of which may be substituted.

In some cases, as disclosed in US patent publication No. 20030195205, PDE9 inhibitors are represented by a compound of formula (XXXV) or a pharmaceutically acceptable salt, solvate or prodrug thereof,

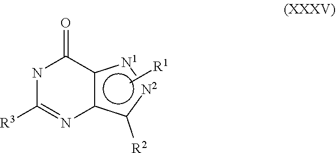

wherein
R¹ is H or C₁₋₆ alkyl, wherein R¹ is attached to either N¹ or N²;
R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹;
R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;
wherein Ar¹ and Ar² are independently groups of formula

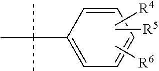

wherein R₄, R₅ and R₆ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and
wherein het¹, het² and het³, which may be the same or different, are aromatic 5-6 membered heterocycles containing 1, 2 or 3 heteroatoms, independently selected from 0, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;
with the provisos that when
   a) R¹ is attached to N¹, R¹ is C₁₋₃ alkyl and R² is propyl then R³ is not methyl substituted by Ar¹, and
   b) R¹ is attached to N¹, R¹ is C₁₋₆ alkyl and R² is methyl then R³ is not C₁₋₄alkyl substituted by Ar¹.

In some cases, as disclosed in WO2003037432, PDE9 inhibitors are represented by a compound of formula (XXXVI),

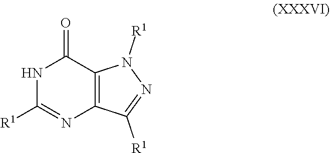

or a pharmaceutically acceptable salt, solvate or prodrug thereof, wherein:
R¹ is H or (C₁-C₆)alkyl;
R² is (C₁-C₆)alkyl, straight chain or branched chain, (C₃-C₇)cycloalkyl or heteroaryl; R³ is (C₁-C₆)alkyl, straight chain or branched chain, optionally substituted by 1-2 groups each independently selected from Ar, (C₃-C₇)cycloalkyl, OAr, SAr, NC(O)(C₁-C₆)alkyl, heteroaryl, xanthene, and naphthalene;
Ar is a group of formula

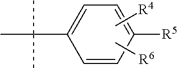

wherein R⁴, R⁵ and R⁶ are each independently selected from H, halo, phenoxy, phenyl, CF₃, OCF₃, S(C₁-C₆)alkyl, (C₁-C₆)alkyl, O(C₁-C₆)alkyl, said alkyl optionally substituted by a heteroaryl group or by a phenyl group, wherein said phenyl group is optionally substituted by 1-3 groups selected from halo, CF₃, OCF₃ and (C₁-C₆)alkyl; or wherein R⁴ and R⁵ may combine to form a (C₂-C₃)alkyl link, wherein said link may optionally incorporate a heteroatom selected from O, S and N; and heteroaryl is aromatic 5-6 membered heterocycle containing 1-3 heteroatoms, each independently selected from O, S and N, said heterocycle optionally substituted by 1-3 substituents, each independently selected from (C₁-C₆)alkyl, halo and phenyl, said phenyl optionally substituted by 1-3 groups selected from halo and (C₁-C₆)alkyl; with the proviso that when R¹ is —CH₃, R² cannot be —CH₂CH₂CH₃.

In some cases, as disclosed in US patent Nos. 10889591 and 11434248, PDE9 inhibitors are represented by a compound of formula (XXXVII),

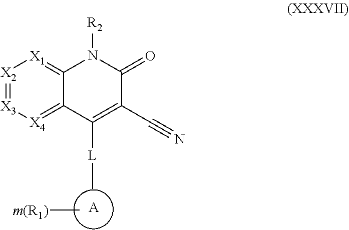

wherein,
   X₁, X₂, X₃, and X₄ are each independently CR₃ or N, and X₁, X₂, X₃, and X₄ are not simultaneously CR₃;
   R³, at each occurrence, is independently selected from the group consisting of hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryloxy, wherein C₁₋₆ alkyl, C₁₋₆ alkoxy, C₁₋₆ alkylamino, (C₁₋₆ alkyl)₂ amino, halogenated C₁₋₆ alkoxy, C₂₋₈ alkenyl, C₂₋₈ alkynyl, C₁₋₆ alkylsulfonyl, C₁₋₆ alkylthio, C₃-6 cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy are unsubstituted or optionally substituted with one or more groups independently selected from the group consisting of hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃-6 cycloalkyl, C₂₋₈ alkynyl, halogenated C₁₋₆ alkyl, C₂₋₈ alkenyl, halogenated C₁₋₆ alkoxy, 4-6 membered heterocyclyl which is unsubstituted or optionally substituted with a substituent, and heteroaryl which is unsubstituted or optionally substituted with a substituent; the substituent in the above 4-6 membered heterocyclyl optionally substituted with a substituent and heteroaryl optionally substituted with a substituent is selected from the group consisting of hydroxy, amino, carboxyl, cyano, nitro, halogen, C₁₋₆ alkyl and C₁₋₆ alkoxy;
L is a bond, —NH—(CH₂)t-, and t is 0, 1, 2 or 3;
   ring A is selected from the group consisting of 3-12 membered heterocyclyl, aryl, 5-10 membered heteroaryl, 3-12 membered cycloalkyl, and 3-12 membered cycloalkenyl, wherein 3-12 membered heterocyclyl has an hetero atom selected from one of O, S, N or any combination thereof, and the S atom may be optionally oxidized to S(O) or S(O)₂, and the 5-10 membered heteroaryl has an hetero atom selected from one of O, S, N or any combination thereof;
each R₁ is independently selected from the group consisting of hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected form the group consisting of hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, and C₁-6 alkylsulfonylamino;
m is 0, 1, 2 or 3; and
   R² is selected from the group consisting of hydrogen, C₁₋₆ alkyl, C₂₋₈ alkenyl, C₂₋₈ alkynyl, and halogenated C₁₋₆ alkyl.

In some embodiments PDE9 inhibitor is selected from the group consisting of:

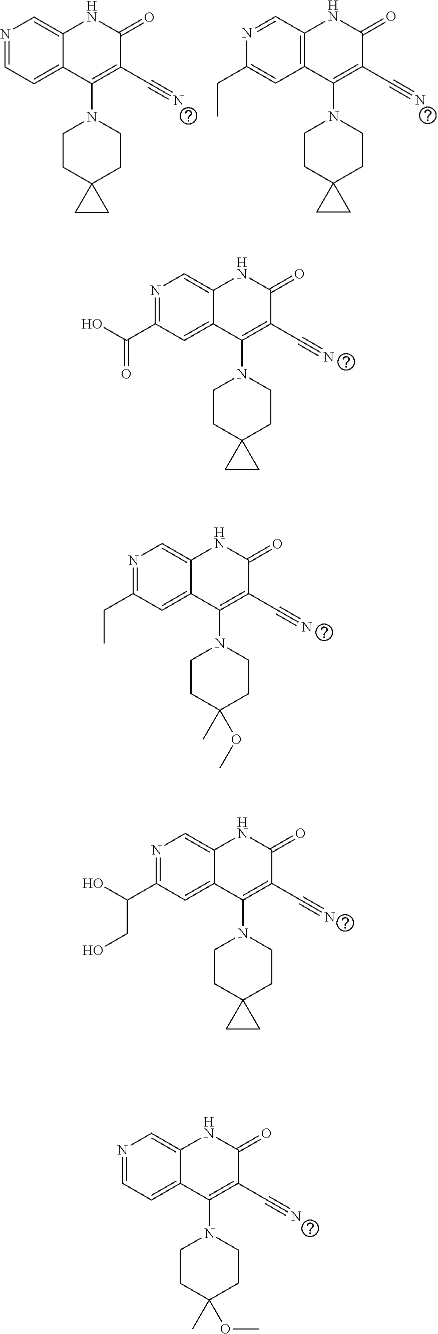

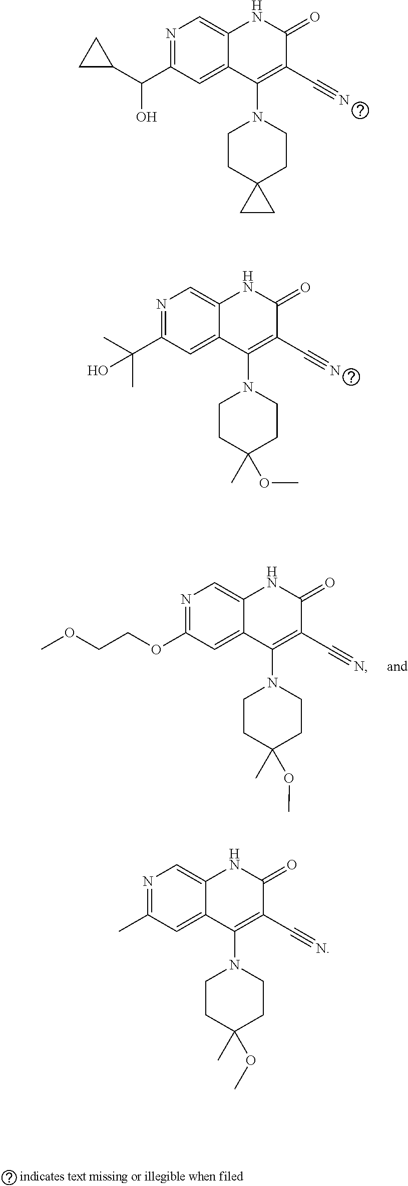 [SMILES: N#Cc1c(N2CCC3(CC2)CC3)c2cc(C(O)C3CC3)ncc2[nH]c1=O ; COC1(C)CCN(c2c(C#N)c(=O)[nH]c3cnc(C(C)(C)O)cc23)CC1 ; COCCOc1cc2c(N3CCC(C)(OC)CC3)c(C#N)c(=O)[nH]c2cn1 ; COC1(C)CCN(c2c(C#N)c(=O)[nH]c3cnc(C)cc23)CC1]

In some embodiments, PDE9 inhibitor is selected from the group consisting of:

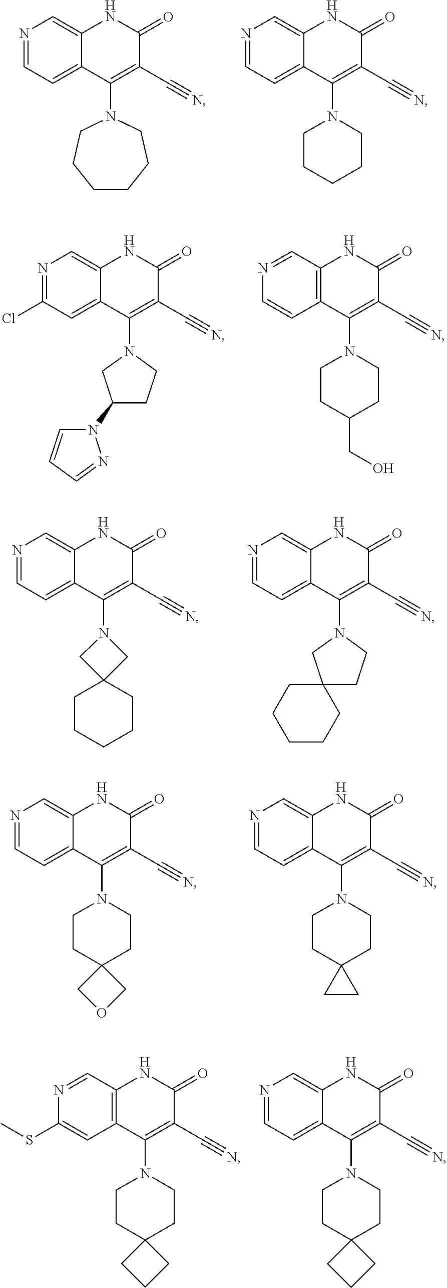

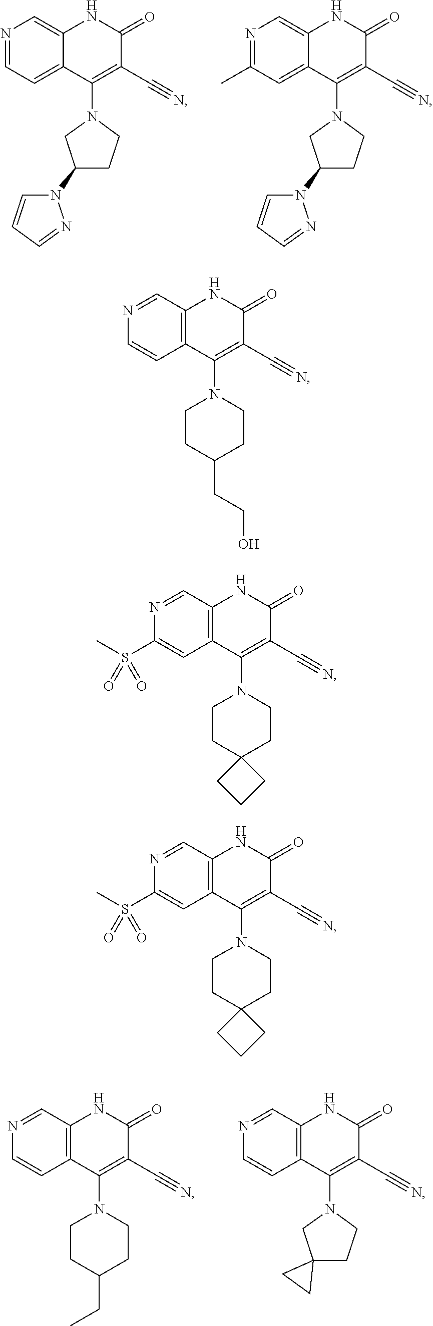

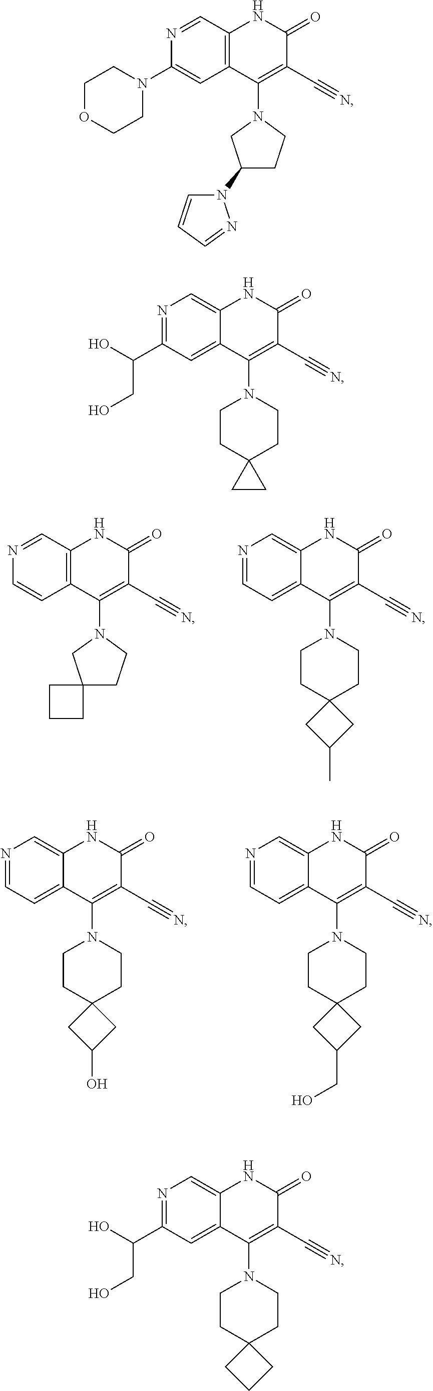

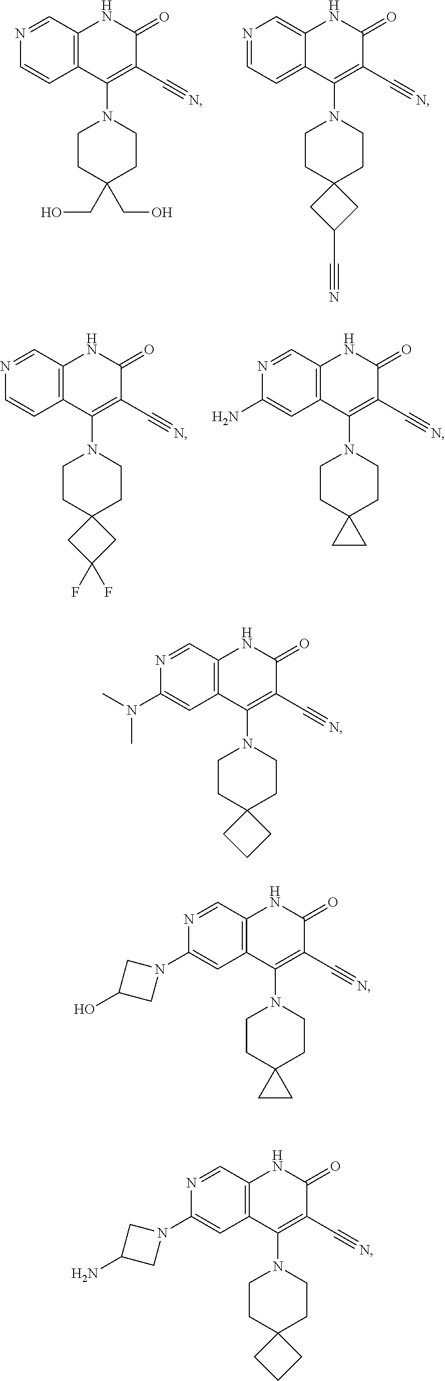

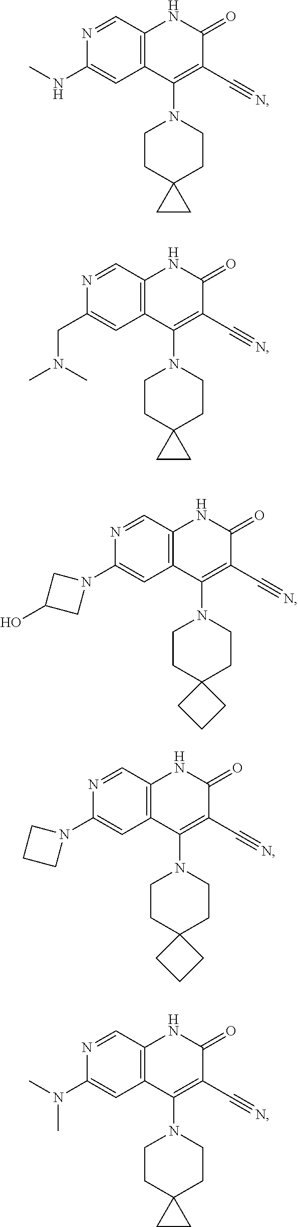

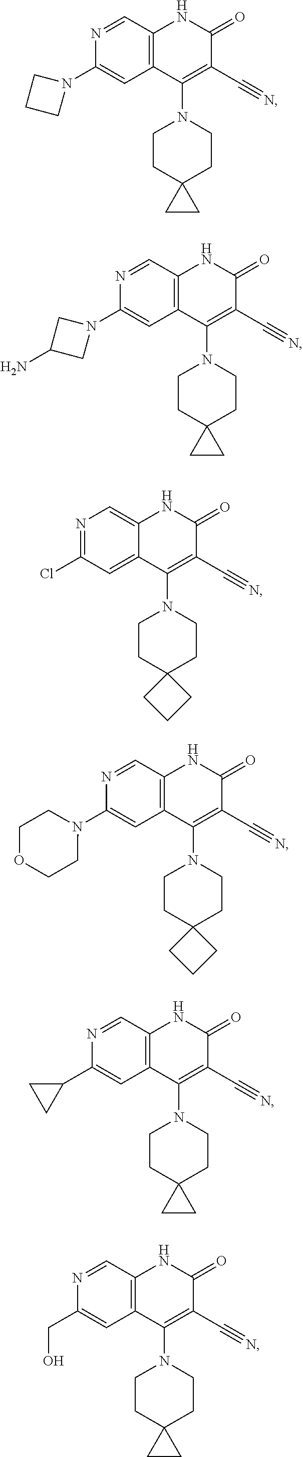

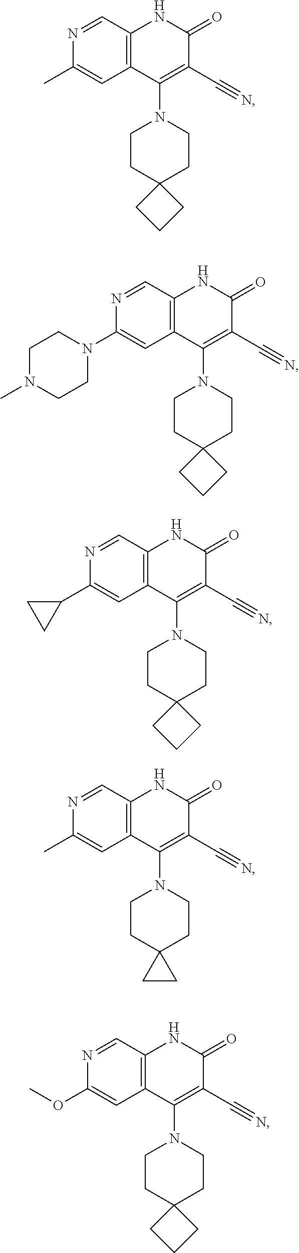

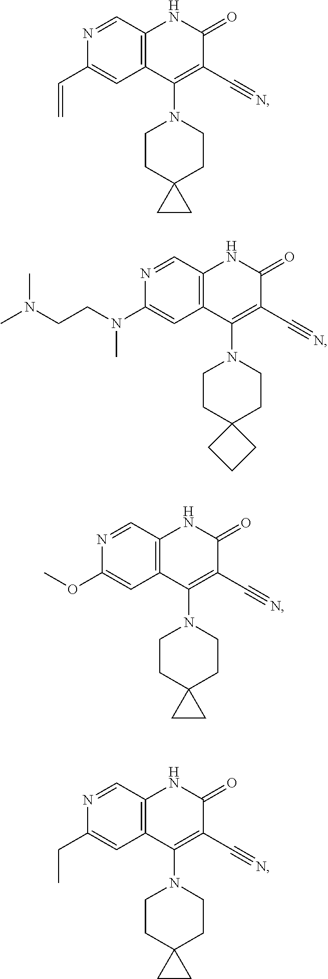 [SMILES: C=Cc1cc2c(N3CCC4(CC3)CC4)c(C#N)c(=O)[nH]c2cn1 ; CN(C)CCN(C)c1cc2c(N3CCC4(CCC4)CC3)c(C#N)c(=O)[nH]c2cn1 ; COc1cc2c(N3CCC4(CC3)CC4)c(C#N)c(=O)[nH]c2cn1 ; CCc1cc2c(N3CCC4(CC3)CC4)c(C#N)c(=O)[nH]c2cn1]

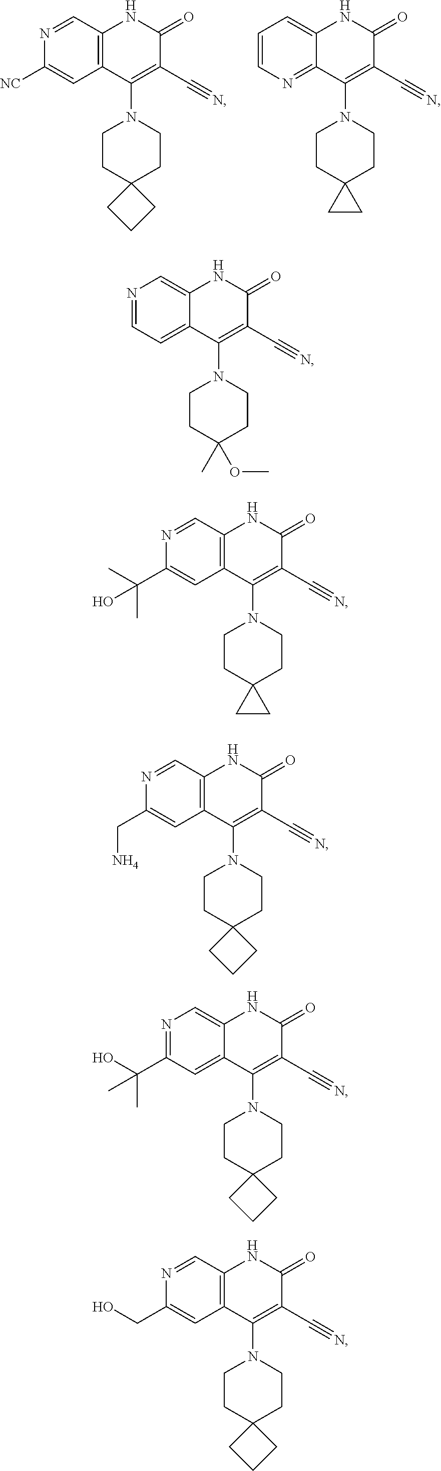

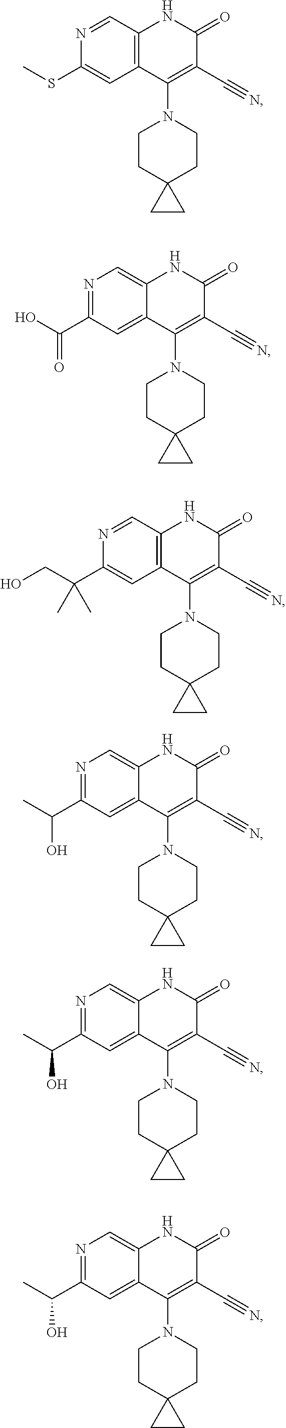

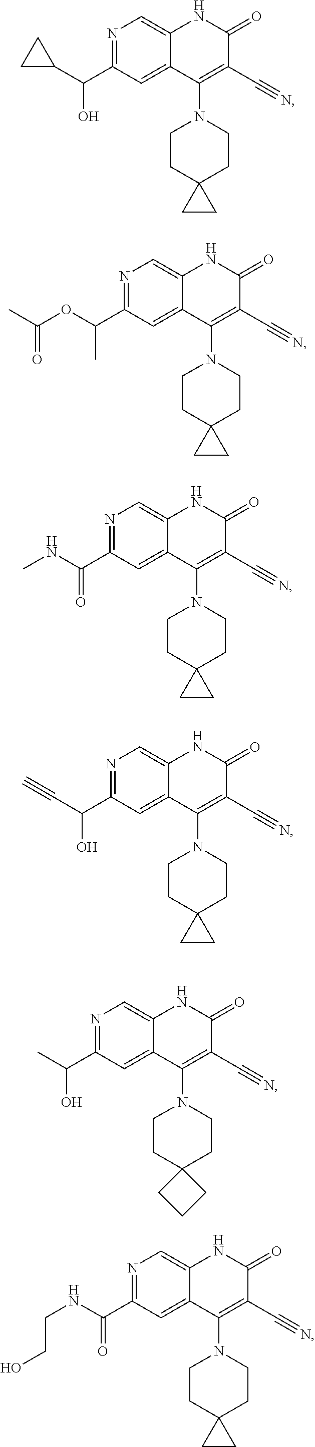

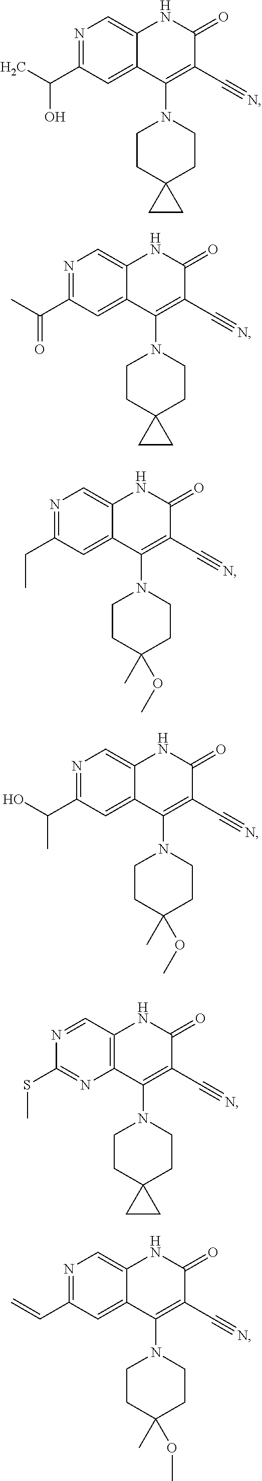

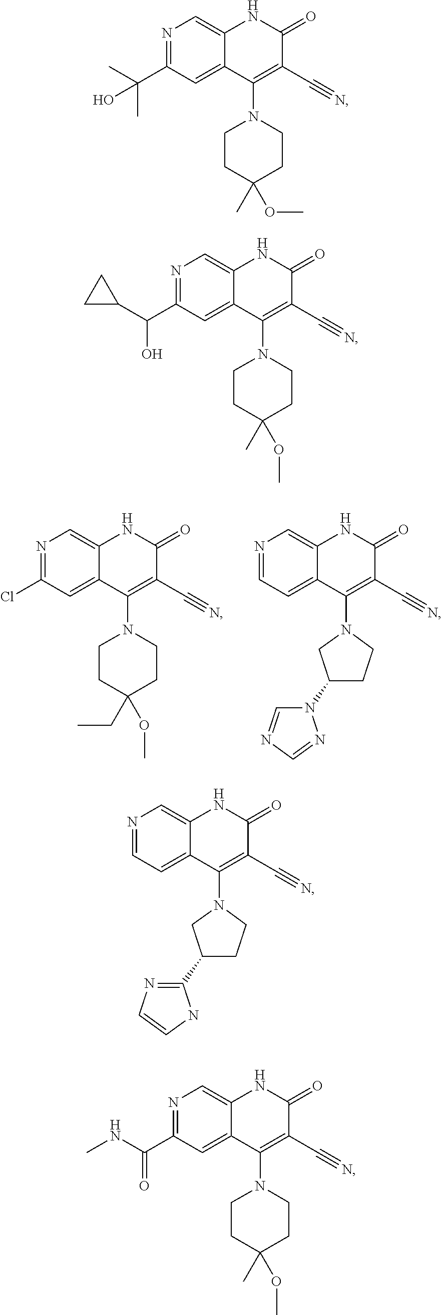

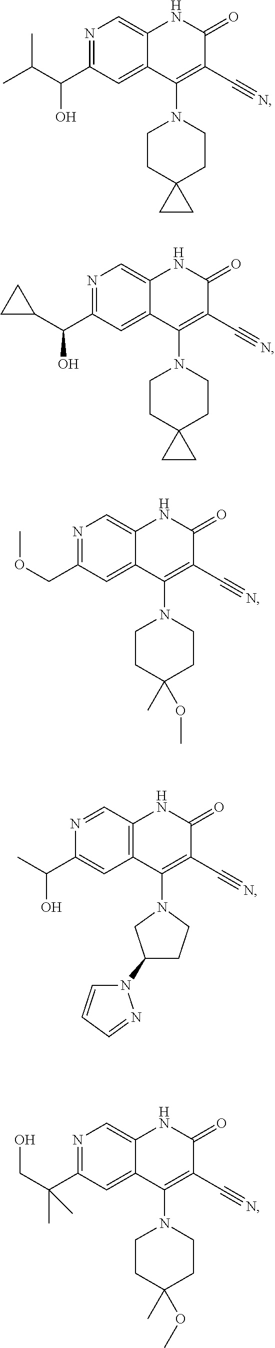 [SMILES: CC(C)C(O)c1cc2c(N3CCC4(CC3)CC4)c(C#N)c(=O)[nH]c2cn1 ; N#Cc1c(N2CCC3(CC2)CC3)c2cc([C@@H](O)C3CC3)ncc2[nH]c1=O ; COCc1cc2c(N3CCC(C)(OC)CC3)c(C#N)c(=O)[nH]c2cn1 ; CC(O)c1cc2c(N3CC[C@@H](n4cccn4)C3)c(C#N)c(=O)[nH]c2cn1 ; COC1(C)CCN(c2c(C#N)c(=O)[nH]c3cnc(C(C)(C)CO)cc23)CC1]

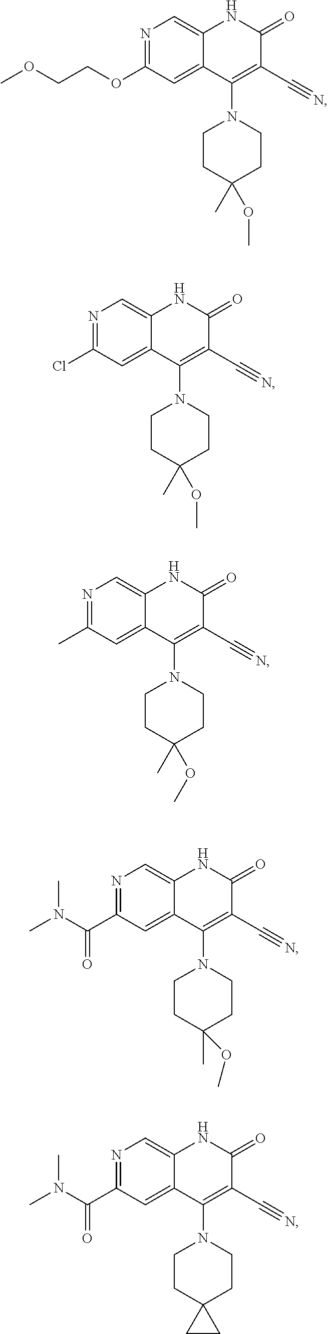 [SMILES: COCCOc1cc2c(N3CCC(C)(OC)CC3)c(C#N)c(=O)[nH]c2cn1 ; COC1(C)CCN(c2c(C#N)c(=O)[nH]c3cnc(Cl)cc23)CC1 ; COC1(C)CCN(c2c(C#N)c(=O)[nH]c3cnc(C)cc23)CC1 ; COC1(C)CCN(c2c(C#N)c(=O)[nH]c3cnc(C(=O)N(C)C)cc23)CC1 ; CN(C)C(=O)c1cc2c(N3CCC4(CC3)CC4)c(C#N)c(=O)[nH]c2cn1]

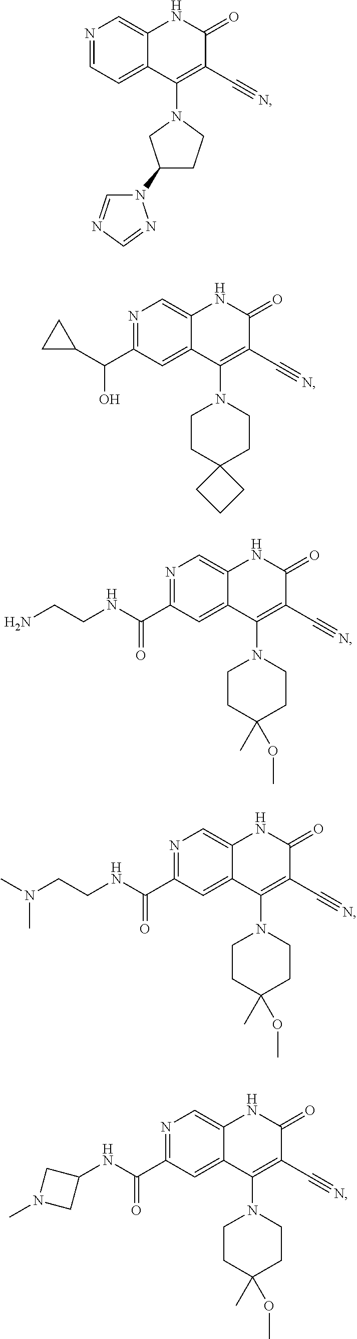

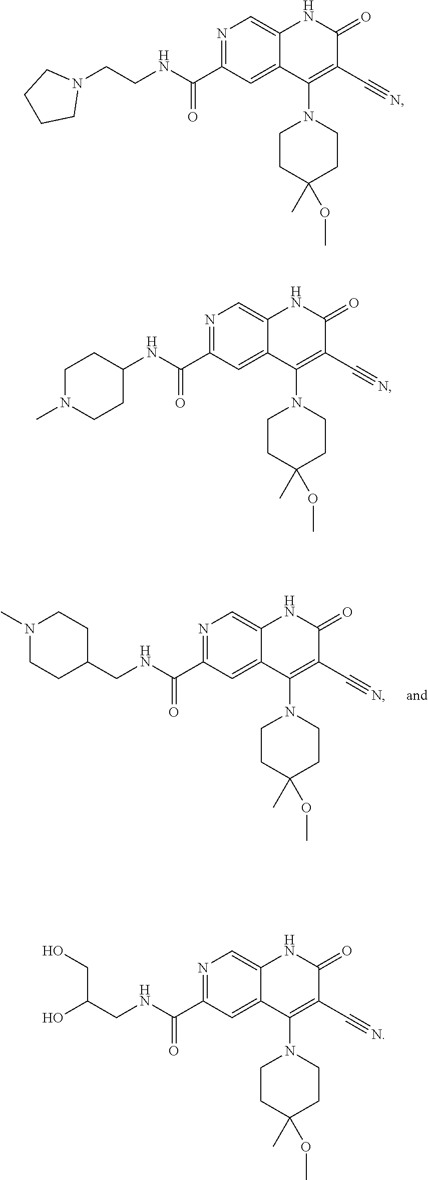 [SMILES: COC1(C)CCN(c2c(C#N)c(=O)[nH]c3cnc(C(=O)NCCN4CCCC4)cc23)CC1 ; COC1(C)CCN(c2c(C#N)c(=O)[nH]c3cnc(C(=O)NC4CCN(C)CC4)cc23)CC1 ; COC1(C)CCN(c2c(C#N)c(=O)[nH]c3cnc(C(=O)NCC4CCN(C)CC4)cc23)CC1 ; "and" ; COC1(C)CCN(c2c(C#N)c(=O)[nH]c3cnc(C(=O)NCC(O)CO)cc23)CC1]

In some embodiments, PDE9 inhibitor is

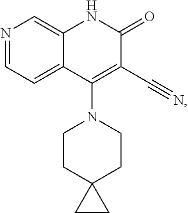 [SMILES: N#Cc1c(N2CCC3(CC2)CC3)c2ccncc2[nH]c1=O]

In some embodiments PDE9 inhibitor is

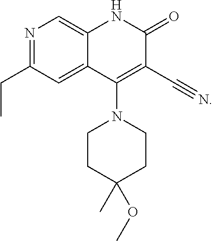 [SMILES: CCc1cc2c(N3CCC(C)(OC)CC3)c(C#N)c(=O)[nH]c2cn1]

In some embodiments, PDE9 inhibitor is

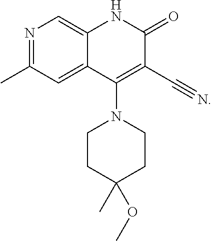 [SMILES: COC1(C)CCN(c2c(C#N)c(=O)[nH]c3cnc(C)cc23)CC1]

In some cases, as disclosed in WO2020/151636, PDE9 inhibitors are represented by a compound of formula (XXXVIII) or its pharmaceutically acceptable salts, isomers, deuterated compounds, metabolites and prodrugs:

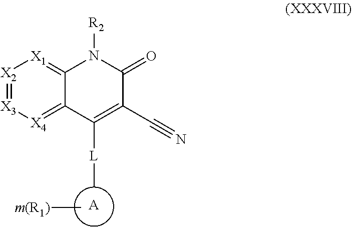

wherein, X₁, X₂, X₄ are independently selected from CR′ or N, X₃ is selected from CR₃ or N, and X₁, X₂, X₃, X₄ are at least one of the N heteroatoms can be optionally oxidized to

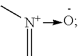

R′ and R₃ are independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclic group, 5-6 membered heteroaryl, aryl, C₁₋₆ alkane Carbonyl, aminocarbonyl, C₁₋₆ alkylaminocarbonyl, (C₁₋₆ alkyl)₂ aminocarbonyl, 4-6 membered heterocyclic carbonyl and 5-6 membered heteroaryl-oxy, wherein said C₁₋₆ alkyl, C₁₋₆ alkoxy, C₁₋₆ alkylamino, (C₁₋₆ alkyl)₂ amino, halogenated C₁₋₆ alkoxy, C₂₋₈ alkenyl, C₂₋₈ alkynyl, C₁₋₆ alkylsulfonyl, C₁₋₆ alkylthio, C₃-6 cycloalkyl, 4-6 membered heterocyclic group, 5-6 membered heteroaryl, aryl, C₁₋₆ alkylcarbonyl, aminocarbonyl, C₁₋₆ alkylaminocarbonyl, (C₁₋₆ alkyl)₂ aminocarbonyl, 4-6 membered heterocyclic carbonyl and 5-6 membered heteroaryl-oxy Unsubstituted or optionally by one or more independently selected from hydroxyl, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyl Oxy, C₃₋₆ cycloalkyl, C₃₋₆ cycloalkylcarbonyloxy, C₂₋₈ alkynyl, halogenated C₁₋₆ alkyl, C₂₋₈ alkenyl, halogenated C₁₋₆ alkoxy,

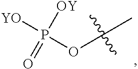

a 4-6 membered heterocyclic group that is unsubstituted or optionally substituted by one or more independent substituents, or a heteroaryl group that is unsubstituted or optionally substituted by one or more independent substituents;
The above-mentioned 4-6 membered heterocyclic group optionally substituted by one or more independent substituents, and the substituent of the heteroaryl group optionally substituted by one or more independent substituents are selected from hydroxyl, amino, carboxyl, cyano, Nitro, halogen, C₁₋₆ alkyl and C₁₋₆ alkoxy;
Y is selected from metal ions or organic ammonium ions; preferably Na⁺, K⁺, NH₄⁺;
L is a bond, —NH—(CH₂)t-, t is 0, 1, 2 or 3;
Ring A is a 3-12 membered heterocyclic group, a 5-10 membered heteroaryl group, a 3-12 membered cycloalkyl group, a 3-12 membered cycloalkenyl group, wherein the heteroatom of the 3-12 membered heterocyclic group is selected from One of O, S, and N or any combination thereof, S atoms can be optionally oxidized to S(O) or S(O)₂, C atoms can be optionally oxidized to C(O), and N heteroatoms can be Is optionally oxidized to

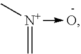

the heteroatom of the 5-10 membered heteroaryl group is selected from one of O, S, and N or any combination thereof;
each R_{1 is} independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkyl Sulfonyl, C₁₋₆ alkylthio, 3-12 membered cycloalkyl, 3-12 membered cycloalkenyl, 3-12 membered heterocyclic group, aryl and 5-10 membered heteroaryl, wherein C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkane Oxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally selected from hydroxyl, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C Substitution of ₁₋₆ alkylsulfonylamino groups;
m is 0, 1, 2 or 3;
R₂ is selected from hydrogen, C₁₋₆ alkyl, C₂₋₈ alkenyl, C₂₋₈ alkynyl, halogenated C₁₋₆ alkyl;
requirement is,
(1) When X 2 is N, X 1 is CH, X₃ is CR₃, and X 4 is CH, ring A is

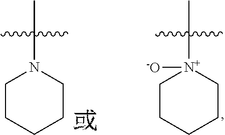

(i) When

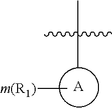

for

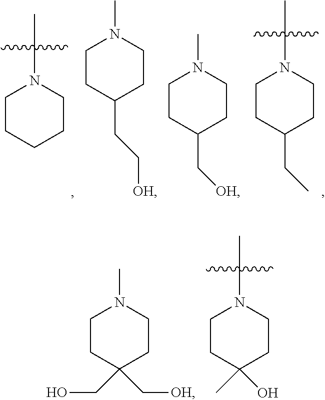

When, R ₃ is not H;
(ii) When

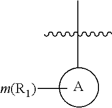

for

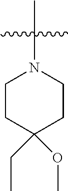 [SMILES: *N1CCC(CC)(OC)CC1]

When, R ₃ is not Cl;
(iii) when

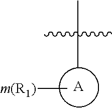

for

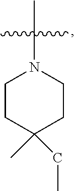 [SMILES: *N1CCC(C)(CC)CC1]

R₃ is not H,

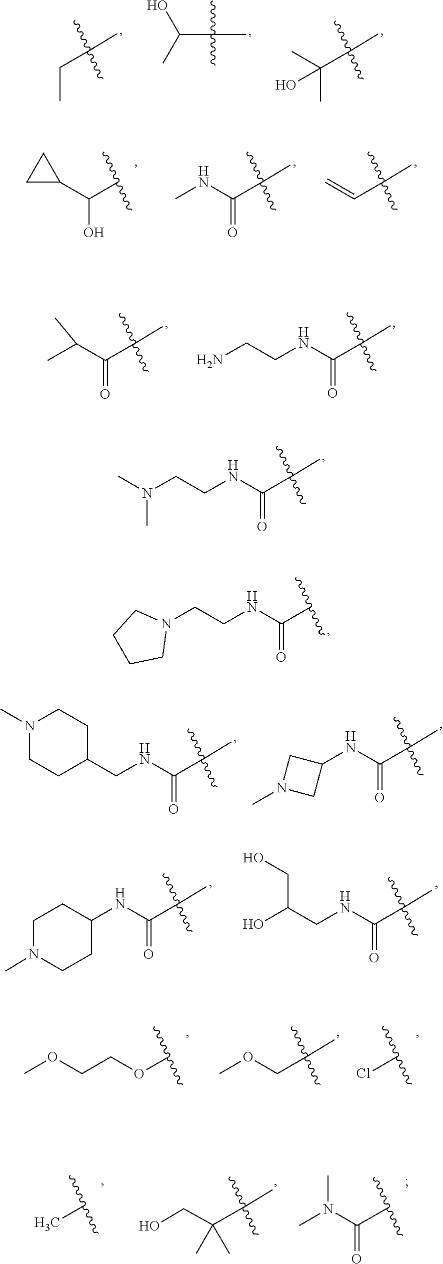

(2) When X₂ and X₄ are N, X ₁ is CH, X ₃ is CR₃, and ring A is

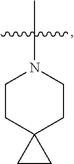 [SMILES: *N1CCC2(CC1)CC2]

When m is 0, R ₃ is not a methylthio group;
(3) When X ₄ is N, X ₁ and X₂ are respectively CH, X ₃ is CR₃, and ring A is

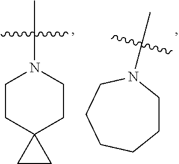

When m is 0, R₃ is not hydrogen;
(4) When X ₁ is N, X ₂ and X₄ are CR′, X ₃ is CR₃, and ring A is pyrrolidinyl,

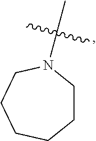 [SMILES: *N1CCCCCC1]

When m is 0, R ₂ is not H or C₁₋₆ alkyl.
Preferably, when X₂ is N, X ₁ is CH, X ₃ is CR₃, and X₄ is CH,

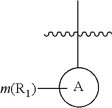

for

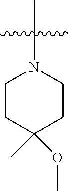 [SMILES: *N1CCC(C)(OC)CC1]

When R_{3 is} selected from isopropyl, cyclopropyl, hydroxymethyl,

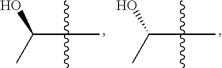

C₁₋₆ alkylcarbonyl, C₁₋₆ alkylcarbonyloxy C₁₋₆ alkyl, is

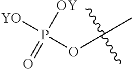

Substituted C₁₋₆ alkyl, C₃₋₆ cycloalkylcarbonyloxy C₁₋₆ alkyl, deuterated C₁₋₆ alkyl; Preferably, when X₂ is N, X ₁ is CH, X ₃ is CR₃, and X₄ is CH, ring A is

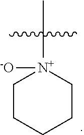

In some cases, as disclosed in WO2020/143626, PDE9 inhibitors are represented by a compound of formula (XXXIX) or a pharmaceutically acceptable salt or isomer thereof:

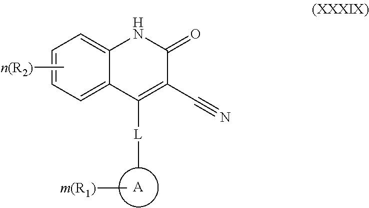

Each R₂ is independently selected from hydrogen, hydroxy, amino, carboxy, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclic group, C₁₋₆ alkylcarbonyl, aminocarbonyl, C₁₋₆ alkylaminocarbonyl, (C₁₋₆ alkyl Group)₂ aminocarbonyl, aryl, 5-6 membered heteroaryl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclic group, C₁₋₆ alkylcarbonyl, aminocarbonyl, C₁₋₆ alkylaminocarbonyl, (C₁₋₆ alkyl)₂ aminocarbonyl, aryl, 5-6 membered heteroaryl, 4-6 membered heterocyclylcarbonyl and 5-6 membered heteroaryl-oxygen The group is unsubstituted or optionally one or more independently selected from hydroxy, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkyl Carbonyloxy, C₃₋₆ cycloalkyl, C₂₋₈ alkynyl, halogenated C₁₋₆ alkyl, C₂₋₈ alkenyl, halogenated C₁₋₆ alkoxy, unsubstituted or any A group substituted by a 4-6 membered heterocyclic group substituted by a substituent, a heteroaryl group which is unsubstituted or optionally substituted by a substituent;
The substituents of the above-mentioned 4-6 membered heterocyclic group optionally substituted by substituents and heteroaryl groups optionally substituted by substituents are selected from the group consisting of hydroxy, amino, carboxy, cyano, nitro, halogen, and C₁₋₆ alkyl Group and C₁₋₆ alkoxy;
L is a bond, —NH—(CH₂)t-, t is 0, 1, 2 or 3;
Ring A is a 3-8 membered monoheterocyclic group, 6-12 membered bridged heterocyclic group, 6-12 membered spiro heterocyclic group, 6-12 membered heterocyclic group, aryl group, 5-10 membered heteroaryl group, 3-12 membered cycloalkyl, 3-12 membered cycloalkenyl, wherein the hetero atom of the heterocyclic group is selected from one of O, S, N or any combination thereof, S atom may be optionally oxidized to S (O) or S(O) 2, the C atom may be optionally oxidized to C(O), and the hetero atom of the 5-10 membered heteroaryl group is selected from one of O, S, N or any combination thereof;
Each R₁ is independently selected from hydrogen, hydroxy, amino, carboxy, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclic group, aryl group and 5-10 membered heteroaryl group are unsubstituted or optionally selected from hydroxy, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ Alkylsulfonylamino group substitution;
m and n are independently 0, 1, 2 or 3;
When ring A is a 3-8 membered monoheterocyclic group, R₂ s not hydrogen;
When ring A is phenyl, L is not a bond;
When ring A is

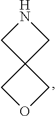

R ₂ is not hydrogen.

In some cases, as disclosed in U.S. patent Ser. No. 10/370,336, PDE9 inhibitors are represented by a compound of formula (XXXX),

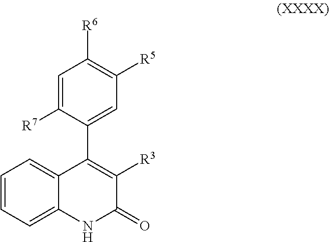

wherein:
   R³ is selected from the group consisting of:
      (1) —CN,
      (2) —CH₃, and
      (3) —CF₃;
   R⁵, R⁶ and R⁷ are independently selected from the group consisting of:
      (1) hydrogen,
      (2) C₁₋₆alkyl, and
      (3) fluoro,
      or R⁵ and R⁶ together with the carbons to which they are attached are joined together to form a fused phenyl ring;
   or a pharmaceutically acceptable salt thereof.

In some cases, as disclosed in U.S. Pat. Nos. 10,934,294 and 11,028,092, PDE9 inhibitors are represented by a compound of formula (XXXXI),

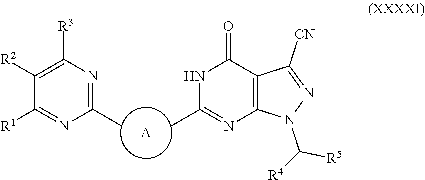

wherein:
   A is a cyclobutyl ring, which is unsubstituted or or substituted with substituents selected from: fluoro and methyl;
   R¹, R² and R³ are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl, and
   (7) —CN;
   or a pharmaceutically acceptable salt thereof.

In some cases, as disclosed in U.S. patent Ser. No. 10/370,337, PDE9 inhibitors are represented by a compound of formula (XXXXII),

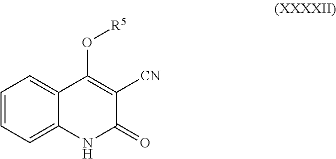

wherein:
R⁵ is selected from the group consisting of
   (1) —C₁₋₆alkyl, which is unsubstituted or substituted with hydroxy, oxetane, or allyl,
   (2) -tetrahydropyranyl, and
   (3) —C₆₋₇cycloalkyl;
   or a pharmaceutically acceptable salt thereof.
      An embodiment herein includes compounds wherein R⁵ is C₁₋₆alkyl, which is unsubstituted or substituted with hydroxy, oxetane, or allyl.

In some cases, as disclosed in US patent publication No. 20220017526, PDE9 inhibitors are represented by a compound of formula (XXXXIII),

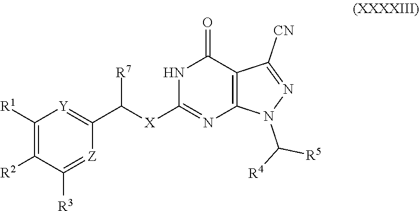

wherein:
   X is —N(R⁶)— or —C(R⁸R⁹)—.
   Y is —N═ or —CH═;
   Z is —N═ or —CH═;
   R¹, R² and R³ are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) azetidine, morpholine, piperidine, or pyrrolidine, and
      (8) —CN;
   R⁶ is selected from:
      (1) hydrogen, and
      (2) C₁₋₆alkyl;
   R⁷ is selected from:
      (1) hydrogen,
      (2) C₁₋₆alkyl, which is unsubstituted or substituted with substituents selected from: hydroxy and fluoro,
      (3) C₂₋₆alkenyl,
      (4) C₃₋₆cycloalkyl, which is unsubstituted or substituted with substituents selected from: hydroxy and fluoro, and
      (5) azetidine, morpholine, piperidine, or pyrrolidine;
      or R⁶ and R⁷ and the —CH—N— to which they are attached are joined to form an azetidine or a pyrrolidine ring;
   R⁸ and R⁹ are independently selected from:
      (1) hydrogen, and
      (2) C₁₋₆alkyl;
      or a pharmaceutically acceptable salt thereof.

In some cases, as disclosed in U.S. patent Ser. No. 10/376,504, PDE9 inhibitors are represented by a compound of formula (XXXXIV),

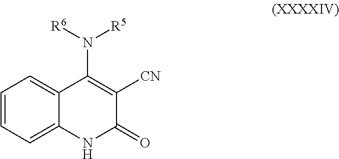

wherein:
R⁵ is selected from the group consisting of:
   (1) hydrogen, and
   (2) —C₁₋₆alkyl;
   R⁶ is —C₁₋₆alkyl, which is unsubstituted or substituted with oxetane;
   or R⁵ and R⁶ are joined together with a —C₃₋₆alkyl through the nitrogen atom to which they are attached to form a azetidine, pyrrolidine, piperidine, or azepan ring, which is unsubstituted or substituted with pyrazole or one or two —C₁₋₆alkyl;
   or a pharmaceutically acceptable salt thereof.

In some cases, as disclosed in US patent publication No. 20220017525, PDE9 inhibitors are represented by a compound of formula (XXXXV),

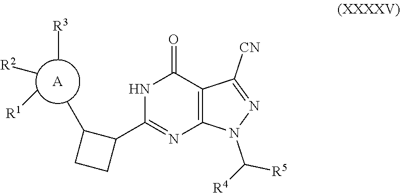

wherein: A is a pyrazinyl, pyrazolyl, pyridyl, pyridyl-N-oxide, 5-pyrimidinyl, pyridazinyl, pyrrolopyrazolyl, dihydropyrrolopyrazolyl, morpholinyl, oxomorpholinyl, or oxadiazolyl ring;
   R¹, R² and R³ as are present are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} as are present are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl, and
      (7) —CN;
      or a pharmaceutically acceptable salt thereof.

In some cases, as disclosed in PCT/EP2015/062140, PDE9 inhibitors are represented by a 1-benzyl-2, 5,6, 8-tetrahydro-3-oxo-2,7-naphthyridine-4-carbonitrile compound of formula (XXXXVI):

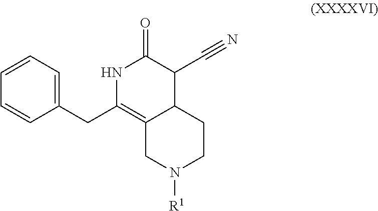

wherein R is —COCH₂R², COO—C₁₋₆-alkyl, CH₂CONR³R⁴, pyridinyl, pyrimidinyl or C₁₋₆-alkyl which is optionally substituted with C₁₋₆-alkoxy, wherein R² is morpholinyl or piperazinyl optionally substituted with C₁₋₆-alkyl,
R³ and R⁴ are independently selected from C₁₋₆-alkyl, and benzyl or R³ and R⁴ together with nitrogen to which they are attached form a morpholinyl or piperazinyl optionally substituted with C₁₋₆-alkyl, and pharmaceutically acceptable acid addition salts thereof.

In some cases, as disclosed in U.S. Pat. No. 8,822,448 and WO2012/033144, PDE9 inhibitors are represented by a compound of formula (XXXXVII), or a pharmaceutically acceptable salt thereof:

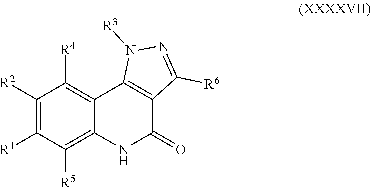

wherein
one of R¹ and R² is hydrogen, halogen, halogeno-lower alkyl, or lower alkyl, —O-lower alkyl or cycloalkyl, each of which may be substituted, and the other is a group of formula (II):

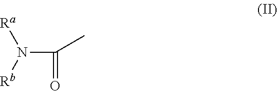

R³ is lower alkyl, cycloalkyl or a saturated hetero ring, each of which may be substituted,
R⁴, R⁵ and R⁶ are the same as or different from each other, and each is hydrogen or lower alkyl, and
R^{a} and R^{b} are the same as or different from each other, and each is hydrogen, or lower alkyl, cycloalkyl, aryl or a hetero ring, each of which may be substituted, or
R^{a} and R^{b} are combined with the adjacent nitrogen atom to form a monocyclic nitrogen-containing hetero ring or a polycyclic nitrogen-containing hetero ring, each of which may be substituted.

In some cases, as disclosed in U.S. Pat. No. 8,822,448 and WO2012/033144, PDE9 inhibitors are represented by a compound of formula (I-1) or a pharmaceutically acceptable salt thereof:

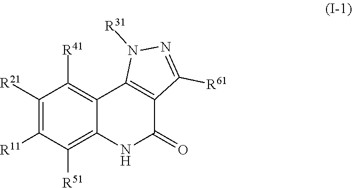

wherein
one of R¹¹ and R²¹ is hydrogen, halogen, or lower alkyl, —O-lower alkyl or cycloalkyl, each of which may be substituted, and the other is a group of formula (II-1):

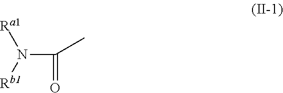

R³¹ is lower alkyl, cycloalkyl or a saturated hetero ring, each of which may be substituted, R⁴¹, R⁵¹ and R⁶¹ are the same as or different from each other, and each is hydrogen or lower alkyl, and
R^{a1} and R^{b1} are the same as or different from each other, and each is hydrogen, or lower alkyl, cycloalkyl, aryl or a hetero ring, each of which may be substituted, or
R^{a1} and R^{b1} are combined with the adjacent nitrogen atom to form a monocyclic nitrogen-containing hetero ring or a polycyclic nitrogen-containing hetero ring, each of which may be substituted.

In some cases, as disclosed in U.S. Pat. No. 9,550,776, PDE9 inhibitors are represented by a compound of formula (XXXXVIII), or a pharmaceutically acceptable salt thereof:

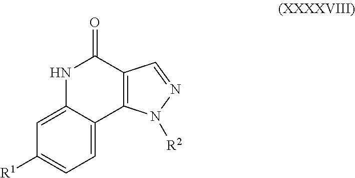

wherein R¹ is a group represented by the formula:

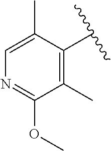 [SMILES: *c1c(C)cnc(OC)c1C]

a group represented by the formula:

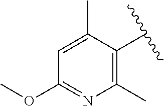

or a group represented by the formula:

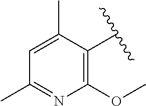

and R² is a 3-methyltetrahydro-2H-pyran-4-yl group or 4-methoxycyclohexyl group.

In some cases, as disclosed in U.S. Pat. No. 9,550,776, PDE9 inhibitors are represented by a compound of formula (XXXXIX):

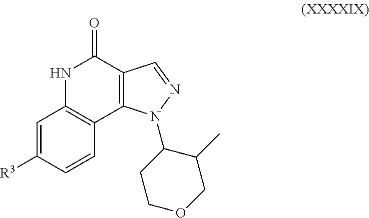

wherein R³ is a group represented by the formula:

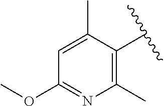

or a group represented by the formula:

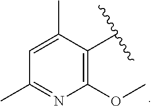

In some cases, as disclosed in U.S. Pat. No. 9,540,380 and WO2012/040230, PDE9 inhibitors are represented by a compound of formula (L),

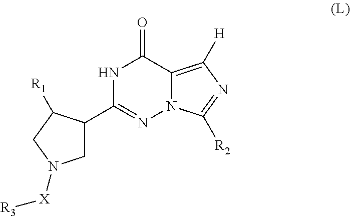

or a pharmaceutically acceptable salt thereof,
wherein:
   X is selected from a bond, C(O) or S(O)₂;
   R¹ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and heterocycloalkyloxy, each of which may be optionally substituted with one or more substituents selected from the group consisting of halogen (e.g., F or CI), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁—C₄)alkyl], (C₁-C₄ alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl];
   R₂ is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen (e.g., F or Cl), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl]; and
   R³ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen (e.g., F or Cl), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl].

In some cases, as disclosed in WO2008/139293, PDE9 inhibitors are represented by a compound of formula (LI),

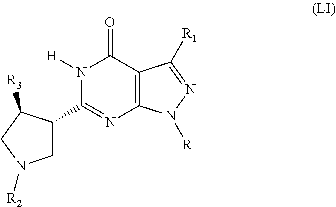

and pharmaceutically acceptable salts thereof,
wherein R is selected from the group consisting of (C₁-C₆)alkyl, (C₂-C₆)alkenyl, (C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl,
R₁ is selected from the group consisting of hydrogen, (C₁-C₄)alkyl, (C₂-C₄)alkenyl, (C₂-C₄)alkynyl, (C₁-C₄)haloalkyl, and cyclopropyl,
R₂ is selected from the group consisting of (C₁-C₆)alkyl, (C₂-C₆)alkenyl, (C₂-C₆)alkynyl, (C₁-C₆)haloalkyl, heteroaryl selected from the group consisting of pyridinyl, pyridazinyl, pyrimidinyl and pyrazinyl, and ER₅, wherein the heteroaryl optionally may be substituted with one to three substituents independently selected from the group consisting of (C₁-C₄)alkyl and (C₁-C₄)haloalkyl,
R₃ is selected from the group consisting of hydrogen, (C₁-C₄)alkyl, (C₂-C₄)alkenyl, (C₂-C₄)alkynyl, (C₃-C₆)cycloalkyl, and (C₁-C₄)haloalkyl,
E is selected from the group consisting of —CH₂—, —CH₂CH₂—, —CH₂CH₂CH₂—, and —C(O)—,
R₅ is selected from the group consisting of (C₃-Cₑ)cycloalkyl, heterocycloalkyl, aryl, aryloxy, and heteroaryl, any of which optionally may be substituted with one to three substituents, such substituents being independently selected from the group consisting of (C₁-C₄)alkyl, (C₂—
C₄)alkenyl, (C₂-C₄)alkynyl, (C₁-C₄)hydroxyalkyl, (C₁-C₄)haloalkyl, (C₁-C₄)alkoxy, (C₁-C₄)haloalkoxy, (C₃-C₈)cycloalkyl, halo, cyano, phenyl, morpholinyl, (C₁-C₄)alkylamino, pyrazolyl, triazolyl, and imidazolyl.
Preferably, R is selected from the group consisting of ethyl, isopropyl, trifluoroethyl, cyclobutyl, cyclopentyl, difluorocyclohexyl, methoxyphenyl, and tetrahydro-2H-pyran-4-yl,
R₁ is hydrogen or methyl, R₂ is methyl, trifluoroethyl, trifluorobutyl, pyrimidinyl, trifluoromethylpyπmidinyl, Or ER₅, R₃ is methyl, ethyl, isopropyl, trifluoromethyl, trifluoroethyl, or cyclopropyl, E is —CH₂— or —C(O)—, and R₅ is selected from the group consisting of substituted or unsubstituted cyclopentyl, morpholinyl, phenyl, naphthyl, benzyloxy, pyrimidinyl, pyndinyl, quinolinyl, quinoxalinyl, pyrazinyl, pyrazolyl, benzimidazolyl, cinnolinyl, naphthydπnyl, pyπdo[2,3-b]pyrazιnyl, ιmιdazo[4,5-c]pyπdinyl, benzothiadiazolyl, tetrahydropyrazolo[1,5-a]pyrιdιnyl, dihydrobenzodioxinyl, imidazolyl, dihydrobenzofuranyl, triazolyl, oxazolyl, isoxazolyl, benzodioxinyl, thiazolyl, ιmιdazo[1,2-a]pyrιdιnyl, tetrahydrobenzothiazolyl, dihydrobenzoxazinyl, tetrahydropyranyl, tetrahydropyrazolo[1,5-a]azepinyl, and dιhydropyrrolo[1,2-b]pyrazolyl.
More preferably, R is selected from the group consisting of isopropyl, cyclobutyl, cyclopentyl, and tetrahydro-2H-pyranyl, R₁ is hydrogen, R₂ is ER₅, R₃ is methyl or ethyl, E is —CH₂—, and R₅ is selected from the group consisting of phenyl, pyιmιdιn-2-yl pyridin-2-yl, pyrazin-2-yl, and 5-methylρyrazιn-2-yl.

In some cases, as disclosed in WO2010/084438, PDE9 inhibitors are represented by a compound of formula (LII),

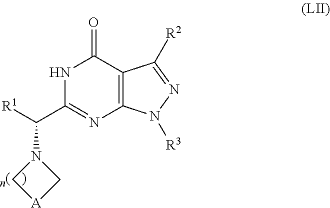

and pharmaceutically acceptable salts thereof, wherein:
R¹ is selected from the group consisting of (i) hydrogen, (ii) (C₁-C₄)alkyl, (iii) (C₂-C₄)alkenyl, (iv) (C₂-C₄)alkynyl, (v) (C₁-C₄)alkoxy, (vi) (C₁-C₄)haloalkyl, (vii) (C₃-C₆)cycloalkyl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl, (viii) 4 to 10 member heterocycloalkyl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl, (ix) aryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl, and (x) heteroaryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl;
R² is selected from the group consisting of hydrogen, (C₁-C₄)alkyl, (C₁-C₄)haloalkyl, cyano, and (C₃-C₆)cycloalkyl; R³ is selected from the group consisting of (C₁-C₆)alkyl, (C₂-C₆)alkenyl,
(C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl; n is 1 or 2;
A is —CR⁴R⁵— or —CHR^{a}—CHR^{b}—;
R⁴ is selected from the group consisting of (i) hydrogen, (ii) (C₁-C₇)alkyl, (iii) (C₃-C₈)cycloalkyl, (iv) 4 to 10 member heterocycloalkyl, (v) aryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl, (vi) heteroaryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl, and (vii) LR⁶
wherein:
L is selected from the group consisting of —CH₂—, —NR⁷—, and —O—; R⁶ is aryl, heteroaryl, (C₁-C₈)alkyl, (C₃-C₈)cycloalkyl, 4 to 10 member heterocycloalkyl, or (C₁-C₈)alkoxy, each of which optionally may be substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl; and
R⁷ is hydrogen, methyl or ethyl;
R⁵ is selected from the group consisting of hydrogen, hydroxyl, (C₁-C₄)alkoxy, halogen, and (C₁-C₆)alkyl; or R⁴ and R⁵, together with the carbon to which they are attached, form a cycloalkyl or heterocycloalkyl ring that optionally incorporates an oxo group and is optionally substituted with (C₁-C₈)alkyl, (C₃-C₈)cycloalkyl, halo, (C₁-C₈)alkoxy, or (C₁-C₃)haloalkyl;
R^{a} is (C₁-C₄)alkoxy or R⁸—O—C(O)—, wherein R⁸ is (C₁-C₄)alkyl; and R^{b} is aryl, heteroaryl, or heterocycloalkyl, optionally substituted with halo, (C₁-C₈)alkyl, (C₃-C₈)cycloalkyl, (C₁-C₈)alkoxy, or (C₁-C₃)haloalkyl; or R^{a} and R^{b}, together with the carbons to which they are attached, form a cycloalkyl or heterocycloalkyl ring that optionally incorporates an oxo group and is optionally substituted with (C₁-C₈)alkyl, (C₃-C₈)cycloalkyl, halo, (C₁-C₈)alkoxy, or (C₁-C₃)haloalkyl.

In some cases, as disclosed in WO2014024125, PDE9 inhibitors are represented by a compound of formula (LIII),

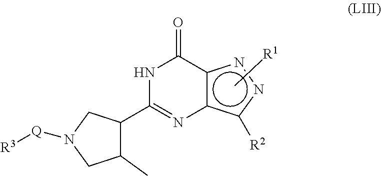

wherein
   R¹ represents hydrogen atom or methyl;
   when R¹ represents hydrogen atom, then R² represents cyclopentyl, tetrahydro-pyranyl, cyclohexyl, or cyclohexyl substituted with 1 or 2 halogen atoms;
   when R¹ represents methyl, then R² represents cyclopentyl;
   R³ is selected from the group consisting of -phenyl unsubstituted or substituted with 1 to 3 substituents selected from F, Cl, Br, I, and OCH₃; and
   -6- to 10-membered heteroaryl with 1 to 3 heteroatoms selected independently from 0, N and S; and Q represents C1-C3-alkylene group, which is unsubstituted or substituted with 1 to 3 C1-C3-alkyl groups; and their salts, including pharmaceutically acceptable salts, and including optically active diastereoisomers and their mixtures.

In some cases, as disclosed in US2004220186, PDE9 inhibitors are represented by a compound of formula (LIV),

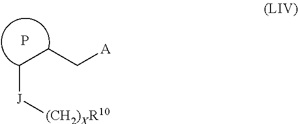

a stereoisomer or prodrug thereof, or a pharmaceutically acceptable salt of said compound, stereoisomer, or prodrug, wherein:
A is:

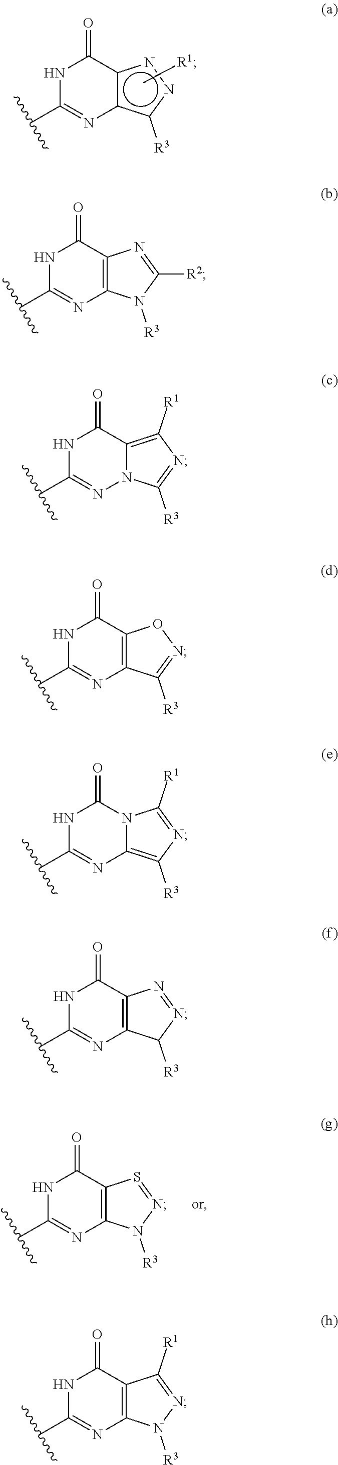

P, including the carbon atoms to which it is attached, is (C₃-C₈)cycloalkyl, (C₃-C₈)heterocycloalkyl, aryl, or heteroaryl; optionally and independently substituted with from 1 to 3 substituents independently selected from halogen, (C₁-C₅)alkyl, (C₁-C₅)alkoxy, and trifluoromethyl;
J is O, S, —N(R¹⁵)—, —N(R¹⁵)CO—, —CON(R¹⁵)—, —SO₂N(R¹⁵)—, or —N(R¹⁵)SO₂—;
x is 0, 1, 2, 3, 4, 5, or 6;
R¹⁰ is —CO₂H, —CON³⁰R³¹, —RN³⁰R³¹, or N(R¹⁵)SO₂R⁴⁰;
R¹ and R² are independently H or (C₁-C₃)alkyl;
R³ is (C₁-C₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C₁-C₅)alkyl, and (C₁-C₅)alkoxy;
R¹⁵ is H or (C₁-C₅)alkyl;
R³⁰ and R³¹ are taken separately and are independently H, (C₁-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰; or
R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C₁-C₅)alkyl, —C₀₂R⁴⁰, —COR⁴⁰, —OR⁴⁰, —CONR⁵⁰R⁵¹, —NR⁵⁰R⁵¹, and —SO₂R⁴⁰;
R⁴⁰ is H, (C₁-C₈)alkyl, (C₃-C₈)cycloalkyl, (C₃-C₈)heterocycloalkyl, aryl, or heteroaryl;
R⁵⁰ and R⁵¹ are taken separately and are independently H, (C₁-C₅)alkyl, (C₃-C₈)cycloalkyl, (C₃-C₈)heterocycloalkyl, aryl, or heteroaryl; or
R⁵⁰ and R⁵¹ are taken together with the nitrogen atom to which they are attached to form a 5- to 8-membered heterocycloalkyl ring, said ring optionally having 1 additional heteroatom independently selected from N, O, and S.

In some cases, as disclosed in WO2003037432, PDE9 inhibitors are represented by a compound of formula (LV)

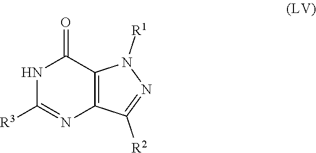

or a pharmaceutically acceptable salt, solvate or prodrug thereof, wherein:
R¹ is H or (C₁-C₆)alkyl;
R² is (C₁-C₆)alkyl, straight chain or branched chain, (C₃-C₇)cycloalkyl or heteroaryl; R³ is (C₁-C₆)alkyl, straight chain or branched chain, optionally substituted by 1-2 groups each independently selected from Ar, (C₃-C₇)cycloalkyl, OAr, SAr, NC(O)(C₁-C₆)alkyl, heteroaryl, xanthene, and naphthalene;
Ar is a group of formula

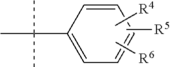

wherein R⁴, R⁵ and R⁶ are each independently selected from H, halo, phenoxy, phenyl, CF₃, OCF₃, S(C₁-C₆)alkyl, (C₁-C₆)alkyl, O(C₁-C₆)alkyl, said alkyl optionally substituted by a heteroaryl group or by a phenyl group, wherein said phenyl group is optionally substituted by 1-3 groups selected from halo, CF₃, OCF₃ and (C₁-C₆)alkyl; or wherein R⁴ and R⁵ may combine to form a (C₂-C₃)alkyl link, wherein said link may optionally incorporate a heteroatom selected from O, S and N; and heteroaryl is aromatic 5-6 membered heterocycle containing 1-3 heteroatoms, each independently selected from O, S and N, said heterocycle optionally substituted by 1-3 substituents, each independently selected from (C₁-C₆)alkyl, halo and phenyl, said phenyl optionally substituted by 1-3 groups selected from halo and (C₁-C₆)alkyl; with the proviso that when R¹ is —CH₃, R² cannot be —CH₂CH₂CH₃.

In some cases, as disclosed in US patent publication No. 20220089593, PDE9 inhibitors are represented by a compound of formula (LVI) or a pharmaceutically acceptable salt, an isomer, a deuterated compound, a metabolite or a prodrug thereof;

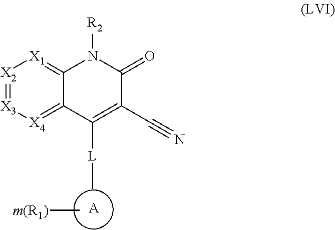

wherein X₁, X₂ and X₄ are each independently selected from CR′ and N; X₃ is selected from CR₃ and N, and at least one of X₁, X₂, X₃ and X₄ is N,
wherein the N heteroatom may be option ally oxidized to

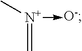

R′ and R₃, at each occurrence, are each independently selected from hydrogen, deuterium, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, 5-6 membered heteroaryl, aryl, C₁₋₆ alkylcarbonyl, aminocarbonyl, C₁₋₆ alkylaminocarbonyl, (C₁₋₆ alkyl)₂aminocarbonyl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂amino, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, 5-6 membered heteroaryl, aryl, C₁₋₆ alkylcarbonyl, aminocarbonyl, C₁₋₆ alkylaminocarbonyl, (C₁₋₆ alkyl)₂aminocarbonyl, 4-6 membered heterocyclylcarbonyl and 5-6 membered heteroaryl-oxy are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino, C₁₋₆ alkylsulfonylamino, C₁₋₆ alkylcarbonyloxy, C₃₋₆ cycloalkyl, C₃-6 cycloalkylcarbonyloxy, C₂₋₈ alkynyl, halogenated C₁₋₆ alkyl, C₂₋₈ alkenyl, halogenated C₁₋₆ alkoxy,

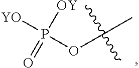

4-6 membered heterocyclyl unsubstituted or optionally substituted with one or more independent substituents, and heteroaryl unsubstituted or optionally substituted with one or more independent substituents;
the substituents for the aforementioned 4-6 membered heterocyclyl optionally substituted with one or more independent substituents and heteroaryl optionally substituted with one or more independent substituents are selected from hydroxy, amino, carboxyl, cyano, nitro, halogen, C₁₋₆ alkyl and C₁₋₆ alkoxy;
Y is selected from metal ions and organic ammonium ions, and is preferably Na⁺, K⁺ or NH₄⁺;
L is a bond or —NH—(CH₂)t-, wherein t is 0, 1, 2 or 3;
ring A is 3-12 membered heterocyclyl, 5-10 membered heteroaryl, 3-12 membered cycloalkyl or 3-12 membered cycloalkenyl, wherein the 3-12 membered heterocyclyl has heteroatoms selected from one of or any combinations of O, S and N, the S atom may be optionally oxidized to S(O) or S(O)₂, the C atom may be optionally oxidized to C(O), the N heteroatom may be optionally oxidized to

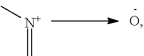

and the 5-10 membered heteroaryl has heteroatoms selected from one of or any combinations of O, S and N;
each R¹ is independently selected from hydrogen, deuterium, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino and C₁₋₆ alkylsulfonylamino;
m is 0, 1, 2 or 3; and
R₂ is selected from hydrogen, C₁₋₆ alkyl, C₂₋₈ alkenyl, C₂₋₈ alkynyl and halogenated C₁₋₆ alkyl;
with the proviso that
(1) when X₂ is N, X₁ is CH, X₃ is CR₃, and X₄ is CH, ring A is

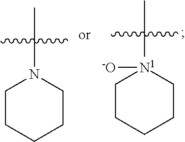

(i) when

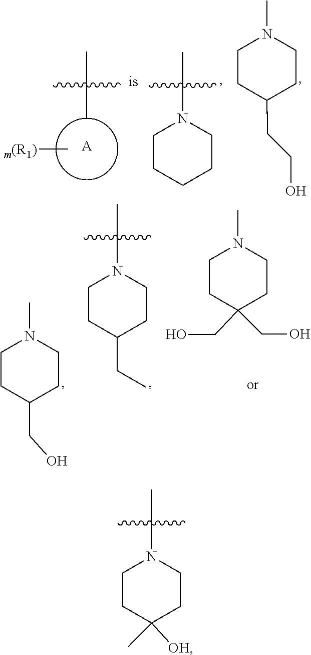

R₃ is not H;

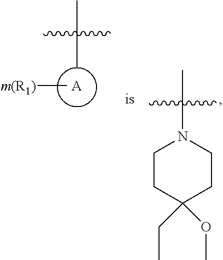

(ii) when

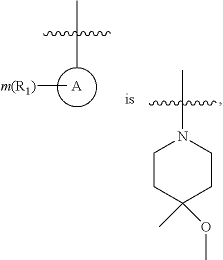

R₃ is not Cl;
(iii) when

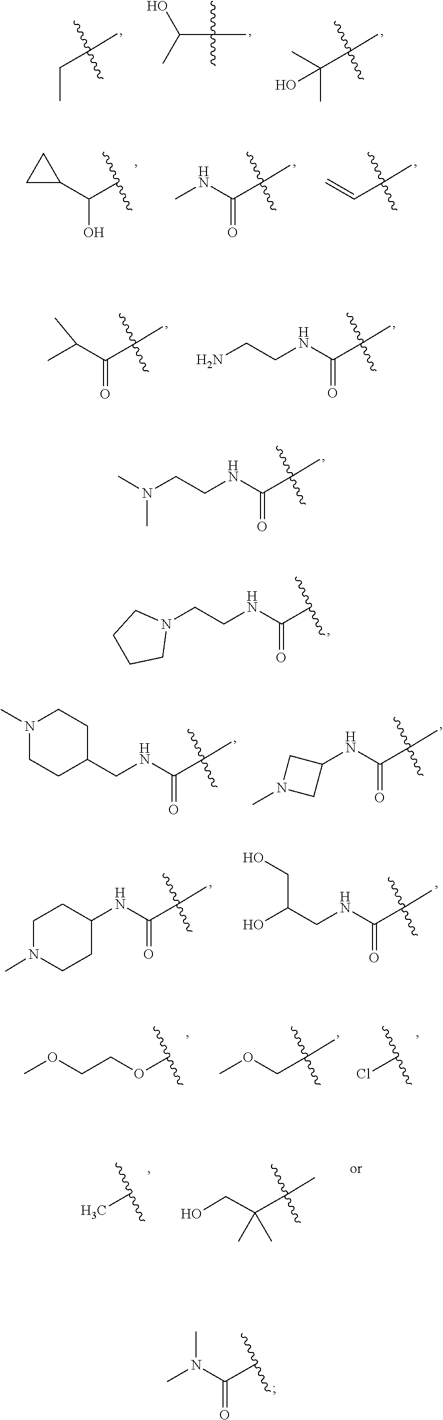

R³ is not H,
(2) when X₂ and X₄ are each N, X₁ is CH, X₃ is CR₃, ring A is

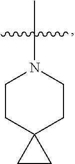 [SMILES: *N1CCC2(CC1)CC2]

and m is 0, R₃ is not methylthio;
(3) when X₄ is N, X₁ and X₂ are each CH, X₃ is CR₃, ring A is

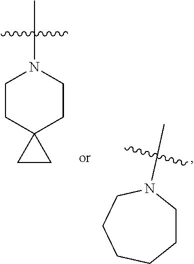 [SMILES: *N1CCC2(CC1)CC2 ; *N1CCCCCC1]

and m is 0, R₃ is not hydrogen; and
(4) when X₁ is N, X₂ and X₄ are CR′, X₃ is CR₃, ring A is pyrrolidinyl or

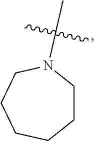 [SMILES: *N1CCCCCC1]

and m is 0, R² is not H or C₁₋₆ alkyl.
Preferably, when X₂ is N, X₁ is CH, X₃ is CR₃, X₄ is CH, and

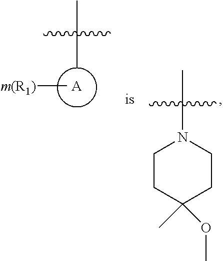

R³ is selected from isopropyl, cyclopropyl, hydroxymethyl,

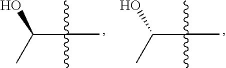

C₁₋₆ alkylcarbonyl, C₁₋₆ alkylcarbonyloxy C₁₋₆ alkyl, C₁₋₆ alkyl substituted with

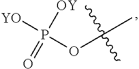

C₃₋₆ cycloalkylcarbonyloxy C₁₋₆ alkyl and deuterated C₁₋₆ alkyl;
preferably, when X₂ is N, X₁ is CH, X₃ is CR₃, and X₄ is CH, ring A is

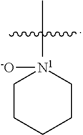

In some cases, as disclosed in U.S. Pat. Nos. 9,643,970 and 9,993,477, PDE9 inhibitors are represented by a compound of formula (LVII) (i.e. a 7H-imidazo[1,5-a]pyrazin-8-one backbone),

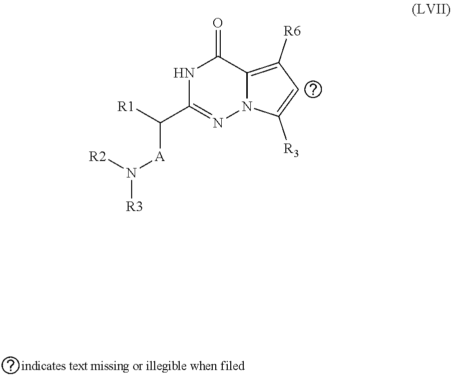

wherein R² is cyclized with either R1 or R3,
wherein R¹, R² and R³ are
R¹, when cyclized with R², is

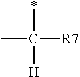

wherein R⁷ is selected from the group consisting of H, —CH₃, —C₂H₅, and —C₃H₇,
wherein * denotes the cyclization point, and
R¹, when not cyclized, is selected from the group consisting of
   H and

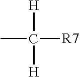

wherein R7 is selected from the group consisting of H, —CH₃, —C₂H₅, and —C₃H₇
R2 is a compound selected from the group consisting of

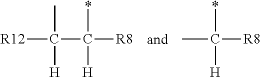

wherein R8 and R12 independently are selected from the group consisting of H,
—CH₃, —C₂H₅, and —C₃H₇
   wherein * denotes the cyclization point, and
R3, when cyclized with R2, is

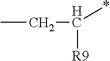

wherein * denotes the cyclization point, and
wherein R9 is selected from the group consisting of H, C₁-C₆ alkyl, branched C₃-C₆ alkyl, C₃-C₆ cycloalkyl, C₆-C₁₀ aryl, substituted C₆-C₁₀ aryl, C₃-C₉heteroaryl, substituted C₃-C₉heteroaryl, C₁-C₆ alkoxy, branched C₃-C₆ alkoxy, C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is

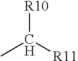

wherein
   R10 is selected from the group consisting of H, —CH₃, and —C₂H₅; and
   R11 is selected from the group consisting of C₆-C₁₀ aryl, substituted C₆-C₁₀ aryl, C₃-C₉heteroaryl, substituted C₃-C₉ heteroaryl
R4 is selected from the group consisting of hydrogen, —CH₃, —C₂H₅, —C₃H₇, —CF₃, —CN, F and Cl;
R5 is selected from the group consisting of C₆-C₁₀ aryl, substituted C₆-C₁₀ aryl, C₃-C₉heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R6 is selected from the group consisting of hydrogen, F, Cl, CN, —CH₃, —C₂H₅, —C₃H₇, and —CF₃;
A is absent or —CH₂—
and tautomers and pharmaceutically acceptable acid addition salts thereof, and polymorphic forms thereof.

In some cases, as disclosed in US patent publication Nos. 20190307754, 20210085684, 20220047589, and 20220023302 and U.S. patent Ser. No. 11/370,795, PDE9 inhibitors are represented by a compound of formula (LVIII) or a pharmaceutically acceptable salt, solvate, or polymorphic thereof:

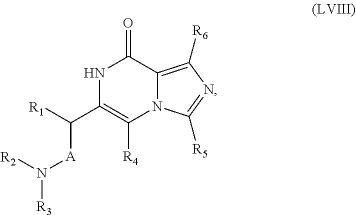

wherein R² is cyclized with either R¹ or R³; wherein R¹, R², and R³ are
R¹, when cyclized with R² is

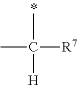

wherein R⁷ is selected from the group consisting of H, —CEE, —C2H5, and C3H7;
wherein * denotes the cyclization point; and
R¹, when not cyclized, is selected from the group consisting of H and

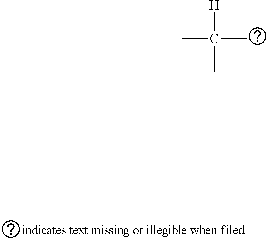

wherein R⁷ is selected from the group consisting of H, —CH₃, —C₂H₅, and C₃H₇;
R² is a compound selected from the group consisting of

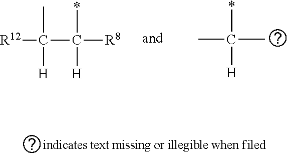

wherein R⁸ and R¹² independently are selected from the group consisting of H, —CH₃, —C₂H₆, and —C₃H₇,
wherein * denotes the cyclization point; and
R³, when cyclized with R² is

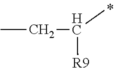

wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryl oxy; and
R³, when not cyclized, is

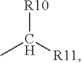

wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C₃-C₉ heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂.

In some embodiments PDE9 inhibitor is selected from the group consisting of:

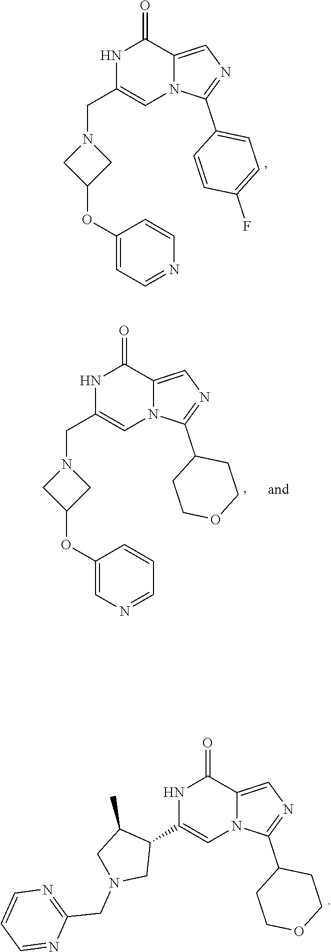 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; C[C@@H]1CN(Cc2ncccn2)C[C@H]1c1cn2c(C3CCOCC3)ncc2c(=O)[nH]1]

In some embodiments, PDE9 inhibitor is

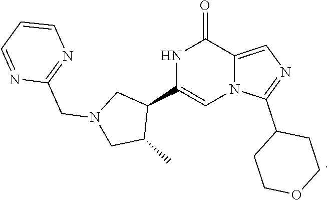 [SMILES: C[C@@H]1CN(Cc2ncccn2)C[C@H]1c1cn2c(C3CCOCC3)ncc2c(=O)[nH]1]

In some cases, as disclosed in U.S. Pat. No. 7,488,733, PDE9 inhibitors are represented by a compound of formula (LIX),

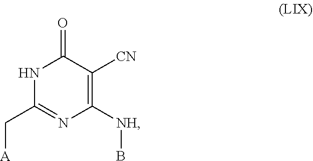

in which
   A is C₁-C₈-alkyl, C₃-C₈-cycloalkyl, tetrahydrofuryl or tetrahydropyranyl, which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
      where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by one or more radicals selected from the group of hydroxy, cyano, halogen, hydroxycarbonyl and a group of the formula —NR³R⁴,
         where
         R³ and R⁴ are independently of one another hydrogen or C₁-C₆-alkyl,
         or
         R³ and R⁴ together with the nitrogen atom to which they are bonded are 5- to 8-membered heterocyclyl,
   B is phenyl or heteroaryl which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, nitro, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
      where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by a radical selected from the group of hydroxy, cyano, halogen, hydroxycarbonyl and a group of the formula —NR³R⁴,
         where
         R³ and R⁴ have the abovementioned meanings,
   and the salts, solvates and/or solvates of the salts thereof.

In some cases, as disclosed in U.S. Pat. Nos. 8,357,688 and 8,674,096, PDE9 inhibitors are represented by a compound of formula (LX),

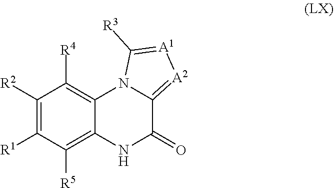

wherein
A¹ is N and A² is CR⁶,
one of R¹ and R² is hydrogen; halogen; or lower alkyl, —O-lower alkyl, or cycloalkyl, each of which may be substituted with one or more substituents selected from the group consisting of —OH, —O-lower alkyl, —NH₂, —NH-lower alkyl, —N(lower alkyl)₂, and a monocyclic nitrogen-containing hetero ring which may be substituted with lower alkyl; and the other is a group of formula (LXI),

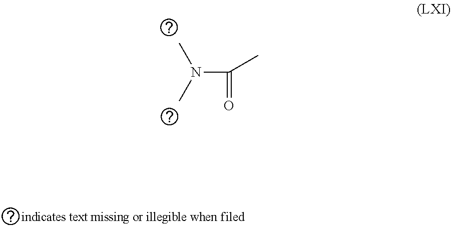

R³ is lower alkyl, cycloalkyl, cycloalkenyl, aryl, heteroaryl, or a saturated hetero ring, each of which may be substituted with one or more substituents selected from the group consisting of halogen, lower alkyl, cycloalkyl, —OH, oxo, —O-lower alkyl, —COOH, —CO—O-lower alkyl, —CO—O-lower alkenyl, —CO—O-lower alkynyl, —CO—O-lower alkylene-O-lower alkyl, —CO—O-lower alkylene-aryl, —CO—O-lower alkylene-O-aryl, —CO—NH₂, —CO—NH-lower alkyl, —CO—N(lower alkyl)₂, —CO—N(lower alkyl)-aryl, —CO—N(lower alkyl)-(lower alkylene-aryl), —CO—NH-lower alkylene-OH, —CO—NH-lower alkylene-CO₂H, and a monocyclic sulfur-containing saturated hetero ring, R⁴ and R⁵ are the same or different, and each is hydrogen or lower alkyl,
R⁶ is hydrogen or lower alkyl, and
R^{a} and R^{b} are combined with the adjacent nitrogen atom to form a polycyclic nitrogen-containing hetero ring, which may be substituted with one or more substituents selected from the group consisting of halogen; —OH; oxo; —O-lower alkyl; cyano; nitro; halogeno-lower alkyl; cycloalkyl; aryl which may be further substituted with a group selected from a group G₁; a hetero ring which may be further substituted with a group selected from a group G₂; lower alkylene-(aryl which may be further substituted with a group selected from a group G₁); lower alkylene-SO₂—NR⁷R⁸; lower alkylene-hetero ring; lower alkyl which may be further substituted with —OH, —O-lower alkyl, cyano, or cycloalkyl; —COOH; —CO—O-lower alkyl; —CO—O-lower alkenyl; —CO—O-lower alkynyl; —CO—O-lower alkylene-O-lower alkyl; —CO—O-lower alkylene-aryl; —CO—O-lower alkylene-O-aryl; —CO—NH₂; —CO—NH-lower alkyl; —CO—N lower alkyl)₂, —CO—N(lower alkyl)-aryl; —CO—N(lower alkyl)-(lower alkylene-aryl); —CO—NH-lower alkylene-OH; —CO—NH-lower alkylene-CO₂H; —NH—SO₂—R⁹; —SO₂—NR⁷R⁸; and a monocyclic nitrogen-containing hetero ring, wherein R⁷ and R⁸ are the same or different, and each is hydrogen or lower alkyl, R⁹ is lower alkyl, or aryl which may be substituted with lower alkyl; group G₁ contains halogen, lower alkyl, halogeno-lower alkyl, —OH, —O-lower alkyl, —O-lower alkylene-aryl, —O-lower alkylene-hetero ring, —O-halogeno-lower alkyl, aryl, and a hetero ring, and group G₂ contains halogen, lower alkyl, halogeno-lower alkyl, —OH, —O-lower alkyl, —O-lower alkylene-aryl, —O-lower alkylene-hetero ring, —O-halogeno-lower alkyl, aryl, and a hetero ring,
or a pharmaceutically acceptable salt thereof.

In some cases, as disclosed in U.S. Pat. Nos. 8,299,080, 8,829,000, & 9040536, PDE9 inhibitors are quinoxaline derivatives or salts thereof. In some cases, quinoxaline derivatives are represented by formula (LXII),

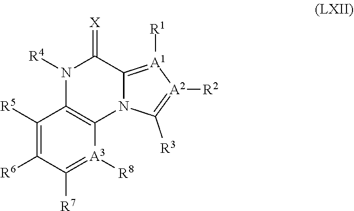

in the formula,
   R¹ and R² each independently stands for hydrogen; halogen; C₁₋₆ alkyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group; acyl which is optionally substituted with hydroxy, C₁₋₆ alkyl, C₂₋₆ alkenyl, C₂₋₆ alkynyl, C₁₋₆ haloalkyl having 1-9 halogen atoms, C₁₋₆ alkoxy, C₁₋₆haloalkoxy having 1-9 halogen atoms, amino, carbocyclic group or heterocyclic group; amino which is optionally substituted with 1-2 substituents selected from C₁₋₆ alkyl, C₂₋₆ alkenyl, C₂₋₆ alkynyl, alkanoyl, carbocyclic group and heterocyclic group; hydroxy; or pyrimidinyl which is optionally substituted with halogen, C₁₋₆ alkyl, C₁₋₆ alkoxy, C₁₋₆ haloalkoxy having 1-9 halogen atoms, nitro or amino,
   R³ stands for C₁₋₉ alkyl, C₂₋₉ alkenyl or C₂₋₉ alkynyl which are optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino group may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); aryl, saturated carbocyclic group or saturated heterocyclic group each of which is optionally substituted with halogen, hydroxy, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with halogen, hydroxy, C₁₋₆ alkoxy, C₁₋₆ haloalkoxy having 1-9 halogen atoms, carboxy, C₁₋₆ alkoxycarbonyl, alkanoyl, amino, amido, carbamoyl, carbocyclic group or heterocyclic group, independently of each other), C₁₋₆ haloalkoxy having 1-9 halogen atoms, carboxy, C₁₋₆ alkoxycarbonyl, alkanoyl, amino, amido, carbamoyl, carbocyclic group or heterocyclic group; carboxy; C₁₋₆ alkoxycarbonyl (here the C₁₋₆ alkoxy moiety in the C₁₋₆ alkoxycarbonyl may further be substituted with hydroxy, halogen, C₁₋₆ alkoxy, C₁₋₆ haloalkoxy having 1-9 halogen atoms, carboxy, C₁₋₆ alkoxycarbonyl, amino, amido, carbamoyl, carbocyclic group or heterocyclic group); amido (here the amino moiety in the amido may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group); or carbamoyl (here the amino moiety in the carbamoyl may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group),
   R⁴ stands for hydrogen; hydroxy; C₁₋₆ alkyl which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; or amino which is optionally substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group,
   R⁵ and R⁸ each independently stands for hydrogen; halogen; C₁₋₆ alkyl, C₂₋₆ alkenyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; cyano; or nitro,
   R⁶ and R⁷ each independently stands for hydrogen; halogen; C₁₋₆ alkyl, C₂₋₆ alkenyl, C₂₋₆ alkynyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); cyano; amino which is optionally substituted with 1-2 substituents selected from C₁₋₆ alkyl, C₂₋₆ alkenyl, C₂₋₆ alkynyl (here the C₁₋₆ alkyl, C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group and heterocyclic group), alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); carbocyclic group or heterocyclic group each of which is optionally substituted with hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, amino, amido or carbamoyl; COR⁹; or SO₂R⁹,
   R⁹ stands for hydrogen; hydroxy; C₁₋₆ alkyl, C₂₋₆ alkenyl, C₂₋₆ alkynyl or C₁₋₆ alkoxy, each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); amino which may be substituted with 1-2 substituents selected from C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl, oxo, carbocyclic group or heterocyclic group), C₁₋₆ haloalkyl having 1-9 halogen atoms, alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); or aziridin-1-yl, azetidin-1-yl, pyrrolidin-1-yl, piperidin-1-yl, piperazin-1-yl, morpholin-1-yl or pyrazol-1-yl, each of which may be substituted with 1-2 substituents selected from hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl), X stands for S or O,
A¹, A² and A³ stand for N or C, independently of each other, with the proviso that R¹, R² and R⁸ are respectively absent where A¹, A² and A³ respectively stand for N,
or salts thereof are provided.

In some cases, as disclosed in U.S. Pat. No. 8,101,624, PDE9 inhibitors are quinazoline derivatives or salts thereof. In some cases, quinazoline derivatives are represented by formula (LXIII),

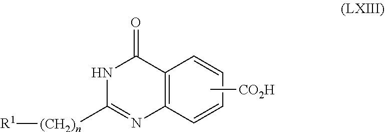

in the formula,
   R¹ stands for phenyl or aromatic heterocyclic group which are optionally substituted with 1-3 substituents selected from halogen, C₁₋₆ alkyl, C₁₋₆ haloalkyl containing 1-6 halogen atoms and C₁₋₆ alkoxy; and
   n is an integer of 1-3
   or salts thereof.

In some cases, as disclosed in CN105669680, PDE9 inhibitors are represented by a compound of formula (LXIV),

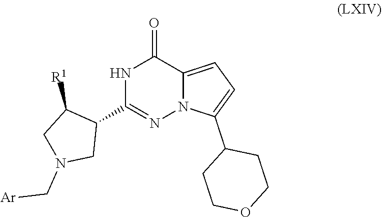

where R′ represents H,

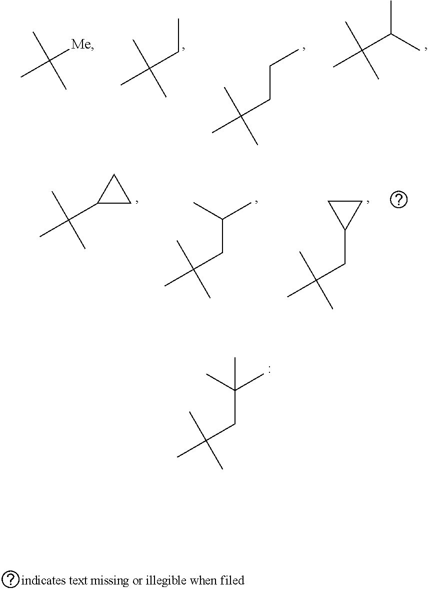

Ar stands for N

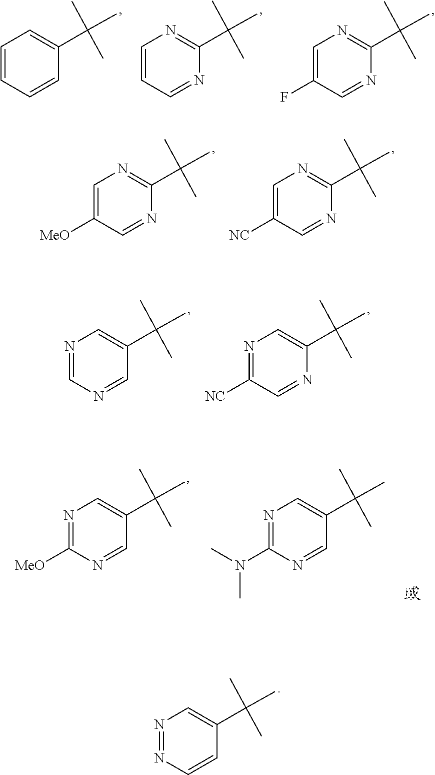

In some cases, as disclosed in WO201424125, PDE9 inhibitors are represented by a compound of formula (LXV),

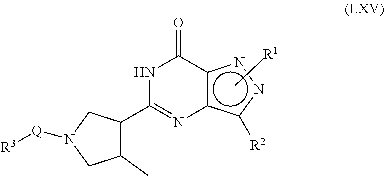

wherein
   R¹ represents hydrogen atom or methyl;
   when R¹ represents hydrogen atom, then R² represents cyclopentyl, tetrahydro-pyranyl, cyclohexyl, or cyclohexyl substituted with 1 or 2 halogen atoms;
   when R¹ represents methyl, then R² represents cyclopentyl;
   R³ is selected from the group consisting of: -phenyl unsubstituted or substituted with 1 to 3 substituents selected from F, Cl, Br, I, and OCH₃; and -6- to 10-membered heteroaryl with 1 to 3 heteroatoms selected independently from O, N and S; and Q represents C1-C3-alkylene group, which is unsubstituted or substituted with 1 to 3 C1-C₃-alkyl groups; and their salts, including pharmaceutically acceptable salts, and including optically active diastereoisomers and their mixtures.

In some embodiments, PDE9 inhibitor is selected from the group consisting of:
5-[(3,4-trans)-1-benzyl-4-methylpyrrolidin-3-yl]-3-cyclopentyl-1-methyl-1H-pyrazolo[4,3-d]pyrimidin-7(6H)-one;
5-((3,4-trans)-1-benzyl-4-methylpyrrolidin-3-yl)-3-cyclopentyl-2-methyl-2H-pyrazolo[4,3-d]pyrimidin-7(6H)-one;
5-[(3,4-trans)-1-benzyl-4-methylpyrrolidin-3-yl]-3-cyclopentyl-1H-pyrazolo[4,3-d]pyrimidin-7(6H)-one;
(−)-5-[(3,4-trans)-1-benzyl-4-methylpyrrolidin-3-yl]-3-cyclopentyl-1H-pyrazolo-[4,3-d]pyrimidin-7(6H)-one;
(+)-5-[(3,4-trans)-1-benzyl-4-methylpyrrolidin-3-yl]-3-cyclopentyl-1H-pyrazolo[4,3-d]pyrimidin-7(6H)-one;
5-[(3,4-trans)-4-methyl-1-(pyrimidin-2-ylmethyl)pyrrolidin-3-yl]-3-(tetrahydro-2H-pyran-4-yl)-1H-pyrazolo[4,3-d]pyrimidin-7(6H)-one;
(−)-5-[(3,4-trans)-4-methyl-1-(pyrimidin-2-ylmethyl)pyrrolidin-3-yl]-3-(tetrahydro-2H-pyran-4-yl)-1H-pyrazolo[4,3-d]pyrimidin-7(6H)-one;
(+)-5-[(3,4-trans)-4-methyl-1-(pyrimidin-2-ylomethyl)pyrrolidin-3-yl]-3-(tetrahydro-2H-pyran-4-yl)-1H-pyrazolo[4,3-d]pyrimidin-7(6H)-one;
3-cyclopentyl-5-[(3,4-trans)-4-methyl-1-(pyrimidin-2-ylmethyl)pyrrolidin-3-yl]-1H-pyrazolo[4,3-d]pyrimidin-7(6H)-one;
5-[1-(3-fluorobenzyl)-4-methylpyrrolidin-3-yl]-3-(tetrahydro-2H-pyran-4-yl)-1H-pyrazolo[4,3-d]pyrimidin-7(6H)-one;
(−)-5-[1-(3-fluorobenzyl)-4-methylpyrrolidin-3-yl]-3-(tetrahydro-2H-pyran-4-yl)-1H-pyrazolo[4,3-d]pyrimidin-7(6H)-one;
(+)-5-[1-(3-fluorobenzyl)-4-methylpyrrolidin-3-yl]-3-(tetrahydro-2H-pyran-4-yl)-1H-pyrazolo[4,3-d]pyrimidin-7(6H)-one; 3-(4,4-DiTluorocyclohexyl)-5-[(3,4-trans)-1-(3-fluorobenzyl)-4-methylpyrrolidin-3-yl]-1H-pyrazolo[4,3-d]pyrimidin-7(6H)-one;
(−)-3-(4,4-Difluorocyclohexyl)-5-[(3,4-trans)-1-(3-fluorobenzyl)-4-methyl-pyrrolidin-3-yl]-1H-pyrazolo[4,3-d]pyrimidin-7(6H)-one;
(+)-3-(4,4-Difluorocyclohexyl)-5-[(3,4-trans)-1-(3-fluorobenzyl)-4-methyl-pyrrolidin-3-yl]-1H-pyrazolo[4,3-d]pyrimidin-7(6H)-one; and
3-(4,4-DiTluorocyclohexyl)-5-[(3,4-trans)-4-methyl-1-(pyrimidin-2-ylmethyl)-pyrrolidin-3-yl]-1H-pyrazolo[4,3-d]pyrimidin-7(6H)-one;
   and their salts, especially pharmaceutically acceptable salts,
   including optically active diastereoisomers thereof and their mixtures, such as racemic mixtures.

In some cases, as disclosed in WO2014016789, PDE9 inhibitors are represented by a compound of formula (LXVI),

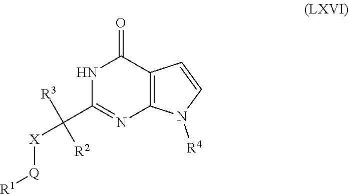

wherein:
   R¹ is selected from the group consisting of:
   -phenyl unsubstituted or substituted with 1 to 3 substituents selected from F, Cl, Br, I, CN, —O—C₁-C₃-alkyl, fluorinated —O—C₁-C₃-alkyl, —(CH₂)ₘOH, and 5-membered heterocyclic group with 1 or 2 heteroatoms selected from N, O and S; and -6- or 10-membered heteroaryl with 1 to 3 heteroatoms selected from O, N and S;
   R² and R³ are independently of each other represent H atom or straight or branched C₁-C₃ alkyl;
   R⁴ is selected from the group consisting of 4- to 6-membered cycloalkyl, wherein one of carbon atoms can be replaced by 0 atom, and which is unsubstituted or substituted with one or two halogen atoms; and straight or branched C₁-C₄ alkyl;
   Q represents a bond or C₁-C₃-alkylene, which can be optionally substituted by one to three C₁-C₃-alkyls;
   X is selected from the group consisting of O, NR⁵, and S(O)ₚ;
   R⁵ represents H atom or C₁-C₃ alkyl;
   m is 1, 2 or 3; p is 0, 1 or 2;
and salts thereof.

In some embodiments, PDE9 inhibitor is selected from the group consisting of:
1-Cyclopentyl-6-(phenoxymethyl)-1H-pyrazolo[3,4-d]pyrimidin-4(5H)-one;
1-Cyclopentyl-6-[(phenylthio)methyl]-1H-pyrazolo[3,4-d]pyrimidin-4(5H)-one;
1-Cyclohexyl-6-(phenoxymethyl)-1H-pyrazolo[3,4-d]pyrimidin-4(5H)-one;
6-(Phenoxymethyl)-1-(tetrahydro-2H-pyran-4-yl)-1H-pyrazolo[3,4-d]pyrimidin-4(5H)-one;
1-isopropyl-6-(phenoxymethyl)-1H-pyrazolo[3,4-d]pyrimidin-4(5H)-one;
1-Tert-butyl-6-(phenoxymethyl)-1H-pyrazolo[3,4-d]pyrimidin-4(5H)-one;
1-Cyclopentyl-6-[(quinolin-8-yloxy)methyl]-1H-pyrazolo[3,4-d]pyrimidin-4(5H)-one;
1-iso-propyl-6-[(quinolin-8-yloxy)methyl]-1H-pyrazolo[3,4-d]pyrimidin-4(5H)-one;
1-Cyclopentyl-6-[(2-methoxyphenoxy)methyl]-1H-pyrazolo[3,4-d]pyrimidin-4(5H)-one;
6-[(2-Bromophenoxy)methyl]-1-cyclopentyl-1H-pyrazolo[3,4-d]pyrimidin-4(5H)-one;
1-Cyclopentyl-6-[(2,4-difluorophenoxy)methyl]-1H-pyrazolo[3,4-d]pyrimidin-4(5H)-one;
1-Cyclopentyl-6-[(2-fluorophenoxy)methyl]-1H-pyrazolo[3,4-d]pyrimidin-4(5H)-one;
6-[(2-Chlorophenoxy)methyl]-1-cyclopentyl-1H-pyrazolo[3,4-d]pyrimidin-4(5H)-one;
3-[(1-Cyclopentyl-4-oxo-4, 5-dihydro-1H-pyrazolo[3,4-d]pyrimidin-6-yl)methoxy]-benzonitrile;
4-[(1-Cyclopentyl-4-oxo-4, 5-dihydro-1H-pyrazolo[3,4-d]pyrimidin-6-yl)methoxy]-benzonitrile;
6-[(3-Chlorophenoxy)methyl]-1-cyclopentyl-1H-pyrazolo[3,4-d]pyrimidin-4(5H)-one;
6-{[(4-Chlorophenyl)thio]methyl}-1-cyclopentyl-1H-pyrazolo[3,4-d]pyrimidin-4(5H)-one;
1-Cyclopentyl-6-[(phenylamino)methyl]-1H-pyrazolo[3,4-d]pyrimidin-4(5H)-one; 1-Cyclopentyl-6-{[methyl(phenyl)amino]methyl}-1H-pyrazolo[3,4-d]pyrimidin-4(5H)-one;
6-[(2-Chlorophenoxy)methyl]-1-(tetrahydro-2H-pyran-4-yl)-1H-pyrazolo[3,4-d]-pyrimidin-4(5H)-one;
1-Cyclopentyl-6-{[2-(trifluoromethoxy)phenoxy]methyl}-1H-pyrazolo[3,4-d]-pyrimidin-4(5H)-one;
1-Cyclopentyl-6-[(2-iodophenoxy)methyl]-1H\hat{ }yrazolo[3,4-d]pyrimidin-4(5H)-one;
6-[(Benzyloxy)methyl]-1-cyclopentyl-1H-pyrazolo[3,4-d]pyn′midin-4(5H)-one;
1-Cyclopentyl-6-{[(2,6-dichlorobenzyl)oxy]methyl}-1H\hat{ }yrazolo[3,4-d]pyrimidin-4(5H)-one;
1-Cyclopentyl-6-{[2-(hydroxymethyl)phenoxy]methyl}-1H-pyrazolo[3,4-d]-pyrimidin-4(5H)-one;
1-Cyclopentyl-6-{[3-(hydroxymethyl)phenoxy]methyl}-1H-pyrazolo[3,4-d]-pyrimidin-4(5H)-one;
6-{[1-(2-Chlorophenyl)ethoxy]methyl}-1-cyclopentyl-1H-pyrazolo[3,4-d]pyrimidin-4(5H)-one;
6-{[(2-Chlorophenyl)amino]methyl}-1-cyclopentyl-1H-pyrazolo[3,4-d]pyrimidin-4(5H)-one;
6-{[2-(1H-imidazol-1-yl)phenoxy]methyl}-1-cyclopentyl-1H-pyrazolo[3,4-d]-pyrimidin-4(5H)-one;
1-Cyclopentyl-6-{[2-(pyrrolidyn-1-yl)phenoxy]methyl}-1H-pyrazolo[3,4-d]-pyrimidin-4(5H)-one;
6-{[2-(1H-pyrrol-1-yl)phenoxy]methyl}-1-cyclopentyl-1H-pyrazolo[3,4-d]pyrimidin-4(5H)-one;
1-Cyclopentyl-6-[(pirydyn-4-yloxy)methyl]-1H-pyrazolo[3,4-d]pyrimidin-4(5H)-one;
1-Cyclopentyl-6-(phenethoxymethyl)-1H-pyrazolo[3,4-d]pyrimidin-4(5H)-one;
1-Cyclopentyl-6-[(phenylsulphinyl)methyl]-1H-pyrazolo[3,4-d]pyrimidin-4(5H)-one;
and 1-Cyclopentyl-6-[(phenylsulphonyl)methyl]-1H-pyrazolo[3,4-d]pyrimidin-4(5H)-one.

In some embodiments, PDE9 inhibitor is 6-[(2-chlorophenoxy)methyl]-1-(4,4-difluorocyclohexyl)-1H-pyrazolo[3,4-d]pyrimidin-4(5H)-one or salts thereof.

In some cases, as disclosed in U.S. Pat. No. 7,488,733, PDE9 inhibitor is represented by a compound of formula (LXVII),

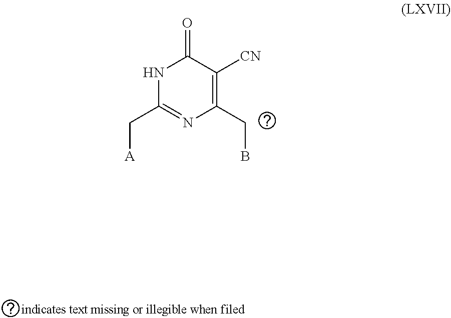

in which
A is C₁-C₈-alkyl, C₃-C₈-cycloalkyl, tetrahydrofuryl or tetrahydropyranyl, which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
   where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by one or more radicals selected from the group of hydroxy, cyano, halogen, hydroxycarbonyl and a group of the formula —NR³R⁴,
      where
      R³ and R⁴ are independently of one another hydrogen or C₁-C₆-alkyl,
      or
      R³ and R⁴ together with the nitrogen atom to which they are bonded are 5- to 8-membered heterocyclyl,
B is phenyl or heteroaryl which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, nitro, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
   where C₁-C₆-alkyl, C₁-C₆-alkoxy, C₁-C₆-alkylamino, C₁-C₆-alkylaminocar-bonyl, C₁-C₆-alkoxycarbonyl, C₁-C₆-alkylcarbonyl, C₁-C₆-alkylsul-fonyl and C₁-C₆-alkylthio are optionally substituted by a radical selected from the group of hydroxy, cyano, halogen, hydroxycarbonyl and a group of the formula —NR³R⁴,
      where
      R³ and R⁴ have the abovementioned meanings,
or salts thereof.

In some cases, PDE9 inhibitor is a compound having a structure of:

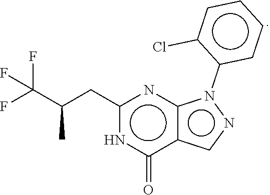

In some cases, PDE9 inhibitor is a compound having a structure of:

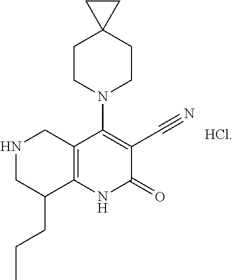 [SMILES: CCCC1CNCc2c1[nH]c(=O)c(C#N)c2N1CCC2(CC1)CC2]

In some cases, as disclosed in U.S. Pat. Nos. 8,088,769 and 8,431,573, PDE9 inhibitor is represented by a compound of formula (LXVIII),

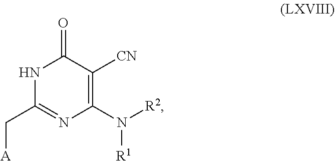

in which
A is phenyl; heteroaryl which is an aromatic, monocyclic radical having 5 to 6 ring atoms and 1 to 3 heteroatoms selected from S, O and N; or a group of the formula

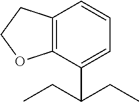

where, when A is phenyl it is substituted by 1 or 2 radicals and when A is heteroaryl it is optionally substituted by up to 2 radicals, where the radicals are independently of one another selected from the group of heteroaryl which is being an aromatic, monocyclic radical having 5 to 6 ring atoms and 1 to 3 heteroatoms selected from S, O and N; halogen; C₁-C₆-alkyl; C₁-C₆-alkoxy; trifluoromethyl; trifluoromethoxy; benzyloxy and benzyl;
   where C₁-C₆-alkyl is optionally substituted by a group of the formula —NR³R⁴ in which R³ is C₁-C₆-alkyl and R⁴ is hydrogen or C₁-C₆-alkoxy(C₁-C₆)alkyl, and heteroaryl is optionally substituted by C₁-C₆-alkoxy,
R¹ is C₃-C₈-cycloalkyl, C₁-C₆-alkyl, C₁-C₆-alkoxy(C—C₆)alkyl, benzyl or a group of the formula

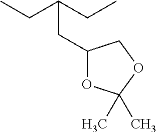

where C₃-C₈-cycloalkyl is optionally substituted by hydroxy, C₁-C₆-alkyl or trifluoromethyl,
C₁-C₆-alkyl is optionally substituted by heteroaryl, C₃-C₈-cycloalkyl or hydroxy, and benzyl is optionally substituted by C₁-C₆-alkoxy or halogen,
R² is hydrogen, or
R¹ and R² together with the nitrogen atom to which they are bonded form a 5- to 6-membered heterocyclyl group which is a monocyclic, saturated or partially unsaturated heterocyclic radical having 5 to 6 ring atoms and 1 to 2 heteroatoms selected from N, O, and S which is optionally substituted by up to 2 substituents independently of one another selected from C₁-C₆-alkyl; hydroxy; cyano; oxo; heteroaryl which is an aromatic, monocyclic radical having 5 to 6 ring atoms and 1 to 3 heteroatoms selected from S, O and N; benzyl; formyl; C₁-C₆-alkylcarbonyl; and the following groups

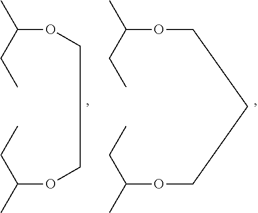

which are linked via the two oxygen atoms to one of the carbon atoms in the heterocycle, where C₁-C₆-alkyl is optionally substituted by hydroxy or heteroaryl which is an aromatic, monocyclic radical having 5 to 6 ring atoms and 1 to 3 heteroatoms selected from S, O and N; or a salt thereof.

In some cases, as disclosed in WO2013142269, PDE9 inhibitor is represented by a compound of formula (AA-I),

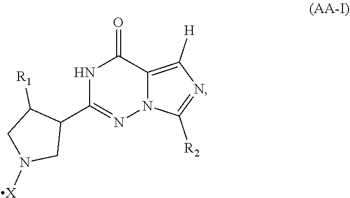

or a pharmaceutically acceptable salt thereof, wherein:
X is selected from the group consisting of a bond, C(O) and S(O)₂;
R¹ is selected from the group consisting of:
   (i) (C₁-C₆) alkyl, which is optionally substituted with one or more R^{a};
   (ii) (C₁-C₆)haloalkyl, which is optionally substituted with one or more R^{a};
   (iii) (C₁-C₆) alkoxy, which is optionally substituted with one or more R^{a};
   (iv) (C₃-C₇) cycloalkyl, which is optionally substituted with one or more R^{b};
   (v) (C₃-C₇) cycloalkyl(C₁-C₄) alkyl, wherein the alkyl portion is optionally substituted with one or more R^{a}, and the cycloalkyl portion is optionally substituted with one or more R^{b};
   (vi) (C₃-C₇) cycloalkyloxy, which is optionally substituted with one or more R;
   (vii) heterocycloalkyl, which is optionally substituted with one or more R^{b};
   (viii) heterocycloalkyl(C₁-C₄)alkyl, wherein the alkyl portion is optionally substituted with one or more R^{a}, and the cycloalkyl portion is optionally substituted with one or more R^{b}; and
   (ix) heterocycloalkyloxy, which is optionally substituted with one or more R^{b};
R² is selected from the group consisting of:
   (i) heterocycloalkyl, which is optionally substituted with one or more R^{c};
   (ii) heterocycloalkyl(C₁-C₄)alkyl, wherein the alkyl portion is optionally substituted with one or more R^{d}, and the cycloalkyl portion is optionally substituted with one or more R^{c};
   (iii) (C₃-C₇) cycloalkyl, which is optionally substituted with one or more R^{c};
   (iv) (C₃-C₇) cycloalkyl(C₁-C₄) alkyl, wherein the alkyl portion is optionally substituted with one or more R^{d}, and the cycloalkyl portion is optionally substituted with one or more R^{c};
   (v) phenyl, which is optionally substituted with one or more R^{e};
   (vi) phenyl(C₁-C₄)alkyl, wherein the alkyl portion is optionally substituted with one or more R^{d}, and the phenyl portion is optionally substituted with one or more R^{e};
   (vii) heteroaryl, which is optionally substituted with one or more R^{e};
   (viii) heteroaryl(C₁-C₄)alkyl, wherein the alkyl portion is optionally substituted with one or more R^{d}, and the heteroaryl portion is optionally substituted with one or more R^{e};
   (ix) (C₁-C₆) alkyl, which is optionally substituted with one or more R^{d}; and
   (x) (C₁-C₆)haloalkyl, which is optionally substituted with one or more R^{d};
R³ is selected from the group consisting of:
   (i) heteroaryl, which is optionally substituted with one or more R;
   (ii) heteroaryl(C₁-C₄)alkyl, wherein the alkyl portion is optionally substituted with one or more R^{g}, and the heteroaryl portion is optionally substituted with one or more R;
   (iii) phenyl, which is optionally substituted with one or more R;
   (iv) phenyl(C₁-C₄)alkyl, wherein the alkyl portion is optionally substituted with one or more R^{g}, and the phenyl portion is optionally substituted with one or more R^{f};
   (v) (C₃-C₇) cycloalkyl, which is optionally substituted with one or more R^{h};
   (vi) (C₃-C₇) cycloalkyl(C₁-C₄) alkyl, wherein the alkyl portion is optionally substituted with one or more R^{g}, and the cycloalkyl portion is optionally substituted with one or more R^{h};
   (vii) heterocycloalkyl, which is optionally substituted with one or more R^{h};
   (viii) heterocycloalkyl(C₁-C₄)alkyl, wherein the alkyl portion is optionally substituted with one or more R^{g}, and the cycloalkyl portion is optionally substituted with one or more R^{h};
   (ix) (C₁-C₆) alkyl, which is optionally substituted with one or more R^{g}; and
   (x) (C₁-C₆)haloalkyl, which is optionally substituted with one or more R^{g}; each occurrence of R^{a}, R^{d}, and R^{g} is independently selected from the group consisting of —S(O)₂(C₁-C₄)alkyl, —OH, —C(O)—(C₁-C₄)alkyl, oxo, —CN, (C₁-C₄)alkoxy, (C₁-C₄)haloalkoxy, (C₃-Cy)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]₂, —NH₂, —NH(C₁-C₄)alkyl, —N[(C₁-C₄)alkyl]₂, —S(O)₂NH(C₁-C₄)alkyl, and —S(O)₂N[(C₁-C₄)alkyl]₂;
each occurrence of R^{a}, R^{d} and R⁹ is independently selected from the group consisting of halo, —S(O)₂(C₁-C₄)alkyl, —OH, —C(O)—(C₁-C₄)alkyl, oxo, —CN, (C₁-C₄)alkoxy, (C₁-C₄)haloalkoxy, (C₃-C₇)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]2, —S(O)₂NH(C₁-C₄)alkyl, and —S(O)₂N[(C₁-C₄)alkyl]2; each occurrence of R^{b}, R^{c}, and R^{h} is independently selected from the group consisting of halo, —S(O)₂(C₁-C₄)alkyl, —OH, —C(O)—(C₁-C₄)alkyl, —C(O)—O—(C₁-C₆)alkyl, —CN, (C₁-C₆)alkyl, (C₁-C₄)haloalkyl, (C₁-C₄)alkoxy, (C₁-C₄)haloalkoxy, (C₃—Cy)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]2, —S(O)₂NH(C₁-C₄)alkyl, and —S(O)₂N[(C₁-C₄)alkyl]2; and
each occurrence of R^{e} and R^{f} is independently selected from the group consisting of halo; —S(O)₂(C₁-C₄)alkyl; —OH; —C(O)—(C₁-C₄)alkyl; —C(O)—O—(C₁-C₆)alkyl; —CN; —C(O)OH; (C₁-C₆)alkyl that is optionally substituted with —OH or —OCH₃; (C₁-C₄)haloalkyl; (C₁-C₄)alkoxy; (C₁-C₄)haloalkoxy; (C₃-Cy)cycloalkyl, that is optionally substituted with from 1-3 substituents independently selected from the group consisting of (C₁-C₄)alkyl; (C₃-C₆)cycloalkyloxy; (C₁-C₄)alkylthio; (C₃-C₆)cycloalkylthio-; —C(O)NH₂; —C(O)NH(C₁-C₄)alkyl; —C(O)N[(C₁-C₄)alkyl]₂; —NH₂; —NH(C₁-C₄)alkyl; —N[(C₁-C₄)alkyl]₂; —NH—C(O)—(C₁-C₄)alkyl; —S(O)₂NH(C₁-C₄)alkyl; —S(O)₂N[(C₁-C₄)alkyl]₂; heterocycloalkyl that is optionally substituted with from 1-3 substituents independently selected from the group consisting of (C₁-C₄)alkyl; phenyl that is optionally substituted with from 1-3 substituents independently selected from the group consisting of —F, —Cl, (C₁-C₄)alkyl, —CF₃, —OCH₃, and —CN; and heteroaryl including from 5 to 6 ring atoms independently selected from the group consisting of N, O, and S, wherein said heteroaryl is optionally substituted with from 1-3 substituents independently selected from the group consisting of (C₁-C₄)alkyl, —CF₃, and —OCH₃.

In some embodiments, PDE9 inhibitor is selected from the group consisting of;
(−)-2-((3,4-trans)-4-methyl-1-(pyrimidin-2-ylmethyl)pyrrolidin-3-yl)-7-(tetrahydrofuran-3-yl)imidazo[5,1-fJ[1,2,4]triazin-4(3H)-one;
(−)-tert-butyl 4-(2-((3,4-trans)-1-benzyl-4-methylpyrrolidin-3-yl)-4-oxo-3,4-dihydroimidazo[1,5-f][1,2,4]triazin-7-yl)piperidine-1-carboxylate;
(+)-tert-butyl 4-(2-((3,4-trans)-1-benzyl-4-methylpyrrolidin-3-yl)-4-oxo-3,4-dihydroimidazo[1,5-f][1,2,4]triazin-7-yl)piperidine-1-carboxylate;
(+)-2-((3,4-trans)-1-benzyl-4-methylpyrrolidin-3-yl)-7-(5-fluoro)-2-methylphenyl)imidazo[1,5-f] [1,2,4]triazin-4(3H)-one;
(−)-2-((3,4-trans)-1-benzyl-4-methylpyrrolidin-3-yl)-7-(5-fluoro-2-methylphenyl)imidazo[1,5-f][1,2,4]triazin-4(3H)-one; (+)-7-(5-fluoro-2-methylphenyl)-2-((3,4-trans)-4-methyl-1-(pyrimidin-2-ylmethyl)pyrrolidin-3-yl)imidazo[1,5-f][1,2,4]triazin-4(3H)-one;
(−)-7-(5-fluoro-2-methylphenyl)-2-((3,4-trans)-4-methyl-1-(pyrimidin-2-ylmethyl)pyrrolidin-3-yl)imidazo[1,5-f][1,2,4]triazin-4(3H)-one;
(−)-2-((3,4-trans)-1-benzyl-4-methylpyrrolidin-3-yl)-7-(piperidin-4-yl)imidazo[1,5-f] [1,2,4]triazin-4(3H)-one;
(+)-2-((3,4-trans)-1-benzyl-4-methylpyrrolidin-3-yl)-7-(piperidin-4-yl)imidazo[1,5-f][1,2,4]triazin-4(3H)-one;
(+)-2-((3,4-trans)-4-methyl-1-(pyrimidin-2-yl)methyl)pyrrolidin-3-yl)-7-(tetrahydrofuran-3-yl)imidazo[5,1-f][1,2,4]triazin-4(3H)-one;
(−)-2-(1-(3,4-trans)-benzyl-4-methylpyrrolidin-3-yl)-7-(tetrahydrofuran-3-yl)imidazo[5,1-f][1,2,4]triazin-4(3H)-one;
(−)-3-(((3,4-trans)-3-methyl-4-(4-oxo-7-(tetrahydro-2H-pyran-4-yl)-3,4-dihydroimidazo[5,1-f][1,2,4]triazin-2-yl)pyrrolidin-1-yl)methyl)benzonitrile;
(+)-4-(((3,4-trans)-3-methyl-4-(4-oxo-7-(tetrahydro-2H-pyran-4-yl)-3,4-dihydroimidazo[5,1-f][1,2,4]triazin-2-yl)pyrrolidin-1-yl)methyl)benzonitrile;
(−)-2-((3,4-trans)-1-((6-methoxypyridin-2-yl)methyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-(2-fluorobenzyl)-4-methylpyrrolidin-3-yl)-7-(tetrahydro-2H-pyran-4-yl)imidazo[5,1-f][1,2,4]triazin-4(3H)-one;
(−)-2-(3,4-trans)-4-methyl-1-(pyridin-2-ylmethyl)pyrrolidin-3-yl)-7-(tetrahydro-2H-pyran-4-yl)imidazo[5,1-f][1,2,4]triazin-4(3H)-one;
(−)-2-((3,4-trans)-1-(3-fluorobenzyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-(4-fluorobenzyl)-4-methylpyrrolidin-3-yl)-7-(tetrahydro-2H-pyran-4-yl)imidazo[5,1-f][1,2,4]triazin-4(3H)-one;
(−)-2-(((3,4-trans)-3-methyl-4-(4-oxo-7-(tetrahydro-2H-pyran-4-yl)-3,4-dihydroimidazo[5,1-f][1,2,4]triazin-2-yl)pyrrolidin-1-yl)methyl)benzonitrile; (−)-2-((3,4-trans)-1-benzyl-4-methylpyrrolidin-3-yl)-7-(furan-3-yl)imidazo[5,1-f][1,2,4]triazin-4(3H)-one;
(+)-2-((3,4-trans)-1-benzyl-4-methylpyrrolidin-3-yl)-7-(furan-3-yl)imidazo[5,1-f][1,2,4]triazin-4(3H)-one;
2-((3,4-trans)-1-benzyl-4-methylpyrrolidin-3-yl)-7-(1-methylpiperidin-4-yl)imidazo[5,1-f][1,2,4]triazin-4(3H)-one;
(+)-2-((3,4-trans)-4-methyl-1-((R)-1-phenylethyl)pyrrolidin-3-yl)-7-(tetrahydro-2H-pyran-4-yl)imidazo[5,1-f][1,2,4]triazin-4(3H)-one;
(−)-2-((3,4-trans)-4-methyl-1-((S)-1-phenylethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(4-(trifluoromethyl)benzyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(3-(trifluoromethyl)benzyl)pyrrolidin-3-yl)-7-(tetrahydro-2H-pyran-4-yl)imidazo[5,1-f][1,2,4]triazin-4(3-H)-one;
(+)-2-((3,4-trans)-1-(3-chlorobenzyl)-4-methylpyrrolidin-3-yl)-7-(tetrahydro-2H-pyran-4-v)imidazo[5,1-f][1,2,4]triazin-4(3H)-one;
(+)-2-((3,4-trans)-1-(2,4-difluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(2-(trifluoromethyl)benzyl)pyrrolidin-3-yl)-7-(tetrahydro-2H-pyran-4-yl)imidazo[5,1-f][1,2,4]triazin-4(3H)-one;
(+)-2-((3,4-trans)-1-(2-fluoro-5-methoxybenzyl)-4-methylpyrrolidin-3-yl)-7-(tetrahydro-2H-pyran-4-yl)imidazo[5,1-f][,2,4]triazin-4(3H)-one;
(+)-2-((3,4-trans)-1-(isoquinolin-7-ylmethyl)-4-methylpyrrolidin-3-yl)-7-(tetrahydro-2H-pyran-4-yl) imidazo[5,1-f][1,2,4]triazin-4(3H)-one;
(+)-2-((3,4-trans)-4-methyl-1-quinolin-6-ylmethyl)pyrrolidin-3-yl)-7-(tetrahydro-2H-pyran-4-yl)imidazo[5,1-f][1,2,4]triazin-4(3H)-one;
(+)-2-((3,4-trans)-1-(2-chlorobenzyl)-4-methylpyrrolidin-3-yl)-7-(tetrahydro-2H-pyran-4-yl)imidazo[5,1-f][1,2,4]triazin-4(3H)-one; (+)-2-((3,4-trans)-1-(4-chlorobenzyl)-4-methylpyrrolidin-3-yl)-7-(tetrahydro-2H-pyran-4-yl)imidazo[5,1-f] [1,2,4]triazin-4(3H)-one;
(+)-2-((3,4-trans)-1-(2,5-difluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(2,3-difluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyrimidin-2-ylmethyl)pyrrolidin-3-yl)-7-(o-tolyl)imidazo[5,1-f][1,2,4]triazin-4(3H)-one;
(+)-2-((3,4-trans)-4-methyl-1-(pyridin-4-ylmethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(quinolin-2-ylmethyl)pyrrolidin-3-yl)-7-(tetrahydro-2H-pyran-4-yl)imidazo[5,1-f][1,2,4]triazin-4(3H)-one;
(+)-2-((3,4-trans)-1-(isoquinolin-6-ylmethyl)-4-methylpyrrolidin-3-yl)-7-(tetrahydro-2H-pyran-4-yl)imidazo[5,1-f][1,2,4]triazin-4(3H)-one;
(−)-2-(3,4-trans)-4-methyl-1-(quinolin-7-ylmethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-((2-oxo-1,2-dihydropyridin-3-yl)methyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyridazin-3-ylmethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyridazin-4-ylmethyl)pyrrolidin-3-yl)-7-(tetrahydro-2H-pyran-4-yl)imidazo[5,1-f][1,2,4]triazin-4(3H)-one; (+)-2-((3,4-trans)-4-methyl-1-(quinazolin-6-ylmethyl)pyrrolidin-3-yl-7-(tetrahydro-2H-pyran-4-yl)imidazo[5,1-f][1,2,4]triazin-4(3H)-one;
(+)-2-((3,4-trans)-4-methyl-1-(quinoxalin-6-ylmethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(quinoxalin-2-ylmethyl)pyrrolidin-3-yl-7-(tetrahydro-2H-pyran-4-yl)imidazo[5,1-f][1,2,4]triazin-4(3H)-one; (+)-2-((3,4-trans)-4-methyl-1-((1-methyl-1H-benzo[d]imidazol-2-yl)methyl)pyrrolidin-3-yl)-7-(tetrahydro-2H-pyran-4-yl)imidazo[5,1-f][1,2,4]triazin-4(3H)-one;
(−)-2-((3,4-trans)-1-((1H-benzo[d]imidazol-2-yl)methyl)-4-methylpyrrolidin-3-1)-7-(tetrahydro-2H-pyran-4-yl)imidazo[5,1-f][1,2,4]triazin-4(3H)-one;
(+)-2-((3,4-trans)-1-(imidazo[1,2-a]pyridin-2-ylmethyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-benzoyl-4-methylpyrrolidin-3-yl)-7-(tetrahydro-2H-pyran-4-yl)imidazo[5,1-f][1,2,4]triazin-4(3H)-one;
(−)-2-((3,4-trans)-1-(2-fluorobenzoyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-(3-fluorobenzoyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-(4-fluorobenzoyl)-4-methylpyrrolidin-3-yl)-7-(tetrahydro-2H-pyran-4-yl)imidazo[5,1-f][1,2,4]triazin-4(3H)-one;
(−)-2-((3,4-trans)-4-methyl-1-nicotinoylpyrrolidin-3-yl)-7-(tetrahydro-2H-pyran-4-yl)imidazo[5,1-f][1,2,4]triazin-4(3H)-one;
(+)-2-((3,4-trans)-1-(4-fluorobenzoyl)-4-methylpyrrolidin-3-yl)-7-(tetrahydro-2H-pyran-4-yl)imidazo[5,1-][1,2,4]triazin-4(3H)-one;
(+)-2-((3,4-trans)-1-benzoyl-4-methylpyrrolidin-3-yl)-7-(tetrahydro-2H-pyran-4-yl)imidazo[5, 1-f][1,2,4]triazin-4(3H)-one;
(+)-2-((3,4-trans)-4-methyl-1-(pyridin-3-ylmethyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(2-methylpyrimidin-4-yl)methyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyrimidin-4-ylmethyl)pyrrolidin-3-yl)-7-(tetrahydro-2H-pyran-4-yl)imidazo[5,1-f][1,2,4]triazin-4(3H)-one;
(−)-2-((3,4-trans)-1-(imidazo [1,2-a]pyrimidin-2-ylmethyl)-4-methylpyrrolidin-3-yl)-7-(tetrahydro-2H-pyran-4-yl)imidazo[5,1-f][1,2,4]triazin-4(3H)-one; (+)-2-((3,4-trans)-1-([1,2,4]triazolo[1,5-a]pyridin-2-ylmethyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(phenylsulfonyl)pyrrolidin-3-yl)-7-(tetrahydro-2H-pyran-4-yl)imidazo[5,1-f][1,2,4]triazin-4(3H)-one;
(+)-2-((3,4-trans)-1-((2-fluorophenyl)sulfonyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-((4-fluorophenyl)sulfonyl)-4-methylpyrrolidin-3-yl)-7-(tetrahydro-2H-pyran-4-yl)imidazo[5,1-f][1,2,4]triazin-4(3H)-one;
(−)-2-(((3,4-trans)-3-methyl-4-(4-oxo-7-(tetrahydro-2H-pyran-4-yl)-3,4-dihydroimidazo [5,1-f][1,2,4]triazin-2-yl)pyrrolidin-1-yl)sulfonyl)benzonitrile;
(−)-2-((3,4-trans)-4-methyl-1-(phenylsulfonyl)pyrrolidin-3-yl)-7-(tetrahydro-2H-pyran-4-yl)imidazo[5,1-f][1,2,4]triazin-4(3H)-one;
(−)-2-((3,4-trans)-1-((4-fluorophenyl)sulfonyl)-4-methylpyrrolidin-3-yl)-7-(tetrahydro-2H-pyran-4-yl)imidazo[5,1-f][1,2,4]triazin-4(3H)-one;
(+)-2-(((3,4-trans)-3-methyl-4-(4-oxo-7-(tetrahydro-2H-pyran-4-yl)-3,4-dihydroimidazo [5,1-f][1,2,4]triazin-2-yl)pyrrolidin-1-yl)sulfonyl)benzonitrile;
(+)-2-((3,4-trans)-4-methyl-1-(pyrimidin-5-ylmethyl)pyrrolidin-3-yl)-7-(tetrahydro-2H-pyran-4-yl)imidazo[5,1-f][1,2,4]triazin-4(3H)-one;
(−)-2-((3,4-trans)-3-methyl-4-(4-oxo-7-(tetrahydro-2H-py ran-4-yl)-3,4-dihydroimidazo [5,1-f][1,2,4]triazin-2-yl)pyrrolidine-1-carbonyl)benzonitrile;
(+)-2-((3,4-trans)-3-methyl-4-(4-oxo-7-(tetrahydro-2H-pyran-4-yl)-3,4-dihydroimidazo [5,1-f][1,2,4]triazin-2-yl)pyrrolidine-1-carbonyl)benzonitrile;
(−)-2-(3,4-trans)-4-methyl-1-(pyrimidine-2-carbonyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyrimidine-2-carbonyl)pyrrolidin-3-yl)-7-(tetrahydro-2H-pyran-4-yl)imidazo[5,1-f][1,2,4]triazin-4(3H)-one;
(−)-2-((3,4-trans)-1-isonicotinoyl-4-methylpyrrolidin-3-yl)-7-(tetrahydro-2H-pyran-4-yl)imidazo[5, 1-f][1,2,4]triazin-4(3H)-one; (−)-4-((3,4-trans)-3-methyl-4-(4-oxo-7-(tetrahydro-2H-pyran-4-yl)-3,4-dihydroimidazo [5,1-f][1,2,4]triazin-2-yl)pyrrolidine-1-carbonyl)benzonitrile;
(−)-2-((3,4-trans)-4-methyl-1-(pyrimidine-5-carbonyl)pyrrolidin-3-yl)-7-(tetrahydro-2H-pyran-4-yl)imidazo[5,1-f][1,2,4]triazin-4(3H)-one;
(−)-2-((3,4-trans)-4-methyl-1-(pyrazine-2-carbonyl)pyrrolidin-3-yl)-7-(tetrahydro-2H-pyran-4-yl)imidazo[5,1-f][1,2,4]triazin-4(3H)-one;
(+)-2-((3,4-trans)-1-((3-fluorophenyl)sulfonyl)-4-methylpyrrolidin-3-yl)-7-(tetrahydro-2H-pyran-4-yl)imidazo[5,1-f][1,2,4]triazin-4(3H)-one;
(+)-4-(((3,4-trans)-3-methyl-4-(4-oxo-7-(tetrahydro-2H-pyran-4-yl)-3,4-dihydroimidazo [5,1-f][1,2,4]triazin-2-yl)pyrrolidin-1-yl)sulfonyl)benzonitrile;
(+)-2-((3,4-trans)-4-methyl-1-(quinolin-7-ylmethyl)pyrrolidin-3-yl)-7-(tetrahydro-2H-pyran-4-yl)imidazo[5,1-f][1,2,4]triazin-4(3H)-one;
(−)-2-((3,4-trans)-4-methyl-1-picolinoylpyrrolidin-3-yl)-7-(tetrahydro-2H-pyran-4-yl)imidazo[5,1-f][1,2,4]triazin-4(3H)-one;
(−)-2-((3,4-trans)-4-methyl-1-(quinoline-2-carbonyl)pyrrolidin-3-yl)-7-(tetrahydro-2H-pyran-4-yl)imidazo[5,1-f][1,2,4]triazin-4(3H)-one;
(−)-2-((3,4-trans)-4-methyl-1-(pyrimidine-4-carbonyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyridin-3-ylsulfonyl)pyrrolidin-3-yl)-7-(tetrahydro-2H-pyran-4-yl)imidazo[5,1-f][1,2,4]triazin-4(3H)-one;
(+)-2-((3,4-trans)-1-(2-methoxybenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3-methoxybenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(4-methoxybenzyl)-4-methylpyrrolidin-3-yl)-7-(tetrahydro-2H-pyran-4-yl)imidazo[5,1-f][1,2,4]triazin-4(3H)-one;
(−)-2-((3,4-trans)-4-methyl-1-(quinazoline-2-carbonyl)pyrrolidin-3-yl)-7-(tetrahydro-2H-pyran-4-yl)imidazo[5,1-f][1,2,4]triazin-4(3H)-one; (+)-2-((3,4-trans)-4-methyl-1-(2-methylbenzyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(3-methylbenzyl)pyrrolidin-3-yl)-7-(tetrahydro-2H-pyran-4-yl)imidazo[5, a -f][1,2,4]triazin-4(3H)-one;
(+)-2-((3,4-trans)-4-methyl-1-(4-methylbenzyl)pyrrolidin-3-yl)-7-(tetrahydro-2H-pyran-4-yl)imidazo[5,1-f][1,2,4]triazin-4(3H)-one;
(−)-2-((3,4-trans)-1-(ethylsulfonyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(ethylsulfonyl)-4-methylpyrrolidin-3-yl)-7-(tetrahydro-2H-pyran-4-yl)imidazo[5,1-f][1,2,4]triazin-4(3H)-one;
(−)-2-((3,4-trans)-1-(cyclohexylmethyl)-4-methylpyrrolidin-3-yl)-7-(tetrahydro-2H-pyran-4-yl)imidazo[5,1-f][1,2,4]triazin-4(3H)-one;
(−)-2-((3,4-trans)-4-methyl-1-(4-(pyrrolidin-1-yl)benzyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(2-(pyrrolidin-1-yl)benzyl)pyrrolidin-3-yl)-7-(tetrahydro-2H-pyran-4-yl)imidazo[5,1-f] [1,2,4]triazin-4(3H)-one;
(+)-2-((3,4-trans)-1-(2-(dimethylamino)benzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3-(dimethylamino)benzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(4-(dimethylamino)benzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-phenethylpyrrolidin-3-yl)-7-(tetrahydro-2H-pyran-4-yl)imidazo[5,1-f][1,2,4]triazin-4(3H)-one;
(+)-2-((3,4-trans)-1-((6-methoxypyridin-3-yl)methyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(pyrimidin-2-yl)pyrrolidin-3-yl)-7-(tetrahydro-2H-pyran-4-yl)imidazo[5,1-f][,2,4]triazin-4(3H)-one; (+)-2-((3,4-trans)-1-(2,6-difluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3-cyclopropylbenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(4-cyclopropylbenzyl)-4-methylpyrrolidin-3-yl)-7-(tetrahydro-2H-pyran-4-yl)imidazo[5, 1-f][1,2,4]triazin-4(3H)-one;
(+)-((3,4-trans)-1,4-dimethylpyrrolidin-3-yl)-7-(tetrahydro-2H-pyran-4-yl)imidazo[5, 1-f][1,2,4]triazin-4(3H)-one;
(−)-2-((3,4-trans)-1-(2-cyclopropylbenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3-(1H-pyrazol-1-yl)benzyl)-4-methylpyrrolidin-3-yl)-7-(tetrahydro-2H-pyran-4-yl)imidazo[5,1-f] [1,2,4]triazin-4(3H)-one;
(+)-2-((3,4-trans)-4-methyl-1-(quinolin-4-ylmethyl)pyrrolidin-3-yl)-7-(tetrahydro-2H-pyran-4-yl)imidazo[5,1-f][1,2,4]triazin-4(3H)-one;
(+)-2-((3,4-trans)-1-((1,3-dimethyl-1H-pyrazol-5-yl)methyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-((2-methylpyrimidin-5-yl)methyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-((2-methylthiazol-5-yl)methyl)pyrrolidin-3-yl)-7-(tetrahydro-2H-pyran-4-yl)imidazo[5, 1-f] [1,2,4]triazin-4(3H)-one;
(+)-2-((3,4-trans)-4-methyl-1-((6-methylpyridin-3-yl)methyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-((5-methylpyrazin-2-yl)methyl)pyrrolidin-3-yl)-7-(tetrahydro-2H-pyran-4-yl)imidazo[5,1-f] [1,2,4]triazin-4(3H)-one;
(+)-2-((3,4-trans)-4-methyl-1-((5-methylpyrazin-2-yl)methyl)pyrrolidin-3-yl)-7-(tetrahydro-2H-pyran-4-yl)imidazo[5,1-f][1,2,4]triazin-4(3H)-one;
(+)-2-((3,4-trans)-4-methyl-1-(3-(pyrrolidin-1-yl)benzyl)pyrrolidin-3-yl)-7-(tetrahydro-2H-pyran-4-yl)imidazo[5,1-f][1,2,4]triazin-4(3H)-one, (+)-2-((3,4-trans)-4-methyl-1-((6-(trifluoromethyl)pyridin-3-yl)methyl)pyrrolidin-3-yl)-7-(tetrahydro-2H-pyran-4-yl)imidazo [5,1-f][1,2,4]triazin-4(3H)-one;
(+)-2-((3,4-trans)-1-((2-ethylpyrimidin-5-yl)methyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3-bromobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(2,4-dichlorobenzyl)-4-methylpyrrolidin-3-yl)-7-(tetrahydro-2H-pyran-4-yl)imidazo[5,1-f][1,2,4]triazin-4(3H)-one;
(+)-2((3,4-trans)-1-(4-bromobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(3,5-dichlorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(3-(trifluoromethoxy)benzyl)pyrrolidin-3-yl)-7-(tetrahydro-2H-pyran-4-yl)imidazo[5,1-f][1,2,4]triazin-4(3H)-one;
(+)-2-((3,4-trans)-1-(2-chloro-4-fluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(2-bromobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4,4-difluorocyclohexyl)imidazo[5,1-f][1,2,4]triazin-4(3H)-one;
(+)-2-((3,4-trans)-1-(4-chloro-3-fluorobenzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-4-methyl-1-(4-(trifluoromethoxy)benzyl)pyrrolidin-3-yl)-7-(tetrahydro-2H-pyran-4-yl)imidazo[5,1-f][1,2,4]triazin-4(3-H)-one;
(+)-2-((3,4-trans)-1-(4-fluoro-3-(trifluoromethyl)benzyl)-4-methylpyrrolidin-3-yl)-7-(tetrahydro-2H-pyran-4-yl)imidazo[5,1-f][1,2,4]triazin-4(3H)-one;
(+)-2-((3,4-trans)-1-(2,5-dichlorobenzyl)-4-methylpyrrolidin-3-yl)-7-(tetrahydro-2H-pyran-4-yl)imidazo[5,1-f][1,2,4]triazin-4(3H)-one; (+)-2-((3,4-trans)-1-(3.5-difluorobenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(3,4-dichlorobenzyl)-4-methylpyrrolidin-3-yl)-7-(tetrahydro-2H-pyran-4-yl)imidazo[1,5-f][1,2,4]triazin-4(3H)-one;
(+)-(4,4-difluorocyclohexyl)-2-((3,4-trans)-1-(4-fluorobenzyl)-4-methylpyrrolidin-3-yl)imidazo[5,1-f][1,2,4]triazin-4(3H)-one;
(+)-2-((3,4-trans)-1-(4-chlorobenzyl)-4-methylpyrrolidin-3-yl)-7-(4,4-difluorocyclohexyl)imidazo[5,1-f][1,2,4]triazin-4(3H)-one;
(+)-7-(4,4-difluorocyclohexyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-2-((3,4-trans)-1-((3aH-benzo[d]imidazol-2-yl)methyl)-4-methylpyrrolidin-3-yl)-7-(4,4-difluorocyclohexyl)imidazo[1,5-f][1,2,4]triazin-4(3H)-one;
(+)-7-(4,4-difluorocyclohexyl)-2-((3,4-trans)-4-methyl-1-(pyridin-2-ylmethyl)pyrrolidin-3-yl)imidazo[1,5-f][1,2,4]triazin-4(3H)-one;
(+)-7-(4,4-difluorocyclohexyl)-2-((3,4-trans)-4-methyl-1-(pyridin-3-ylmethyl)pyrrolidin-3-yl)imidazo[1,5-f][1,2,4]triazin-4(3H)-one;
(+)-7-(4,4-difluorocyclohexyl)-2-((3,4-trans)-4-methyl-1-(pyrazin-2-ylmethyl)pyrrolidin-3-yl)imidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-(5-fluoro-2-methylphenyl)imidazo[1,5-f][1,2,4]triazin-4(3H)-one;
(+)-2-((3,4-trans)-1-(4-chlorobenzyl)-4-methylpyrrolidin-3-yl)-7-(5-fluoro-2-methylphenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(5-fluoro-2-\hat{ }4(3H)-one;
(+)-7-(5-fluoro-2-methylphenyl)-2-((3,4-trans)-1-(4-fluorobenzyl)-4-methylpyrrolidin-3-yl)imidazo[1,5-f][1,2,4]triazin-4(3H)-one;
(+)-7-(5-fluoro-2-methylphenyl)-2-((3,4-trans)-1-(imidazo [1,2-a]pyridin-2-ylmethyl)-4-methylpyrrolidin-3-yl)imidazo[1,5-f][1,2,4]triazin-4(3H)-one (+)-7-(5-fluoro-2-methylphenyl)-2-((3,4-trans)-4-methyl-1-(pyrazin-2-ylmethyl)pyrrolidin-3-yl)imidazo[1,5-f][1,2,4]triazin-4(3H)-one;
(+)-7-(5-fluoro-2-methylphenyl)-2-((3,4-trans)-4-methyl-1-(pyridin-2-ylmethyl)pyrrolidin-3-yl)imidazo[1,5-f][1,2,4]triazin-4(3H)-one;
(+)-(+)-7-(5-fluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-(4-fluorocyclohexyl)imidazo[1,5-f][1,2,4]triazin-4(3H)-one;
(+)-2-((3,4-trans)-1-(4-chlorobenzyl)-4-methylpyrrolidin-3-yl)-7-(4-fluorocyclohexyl)imidazo[1,5-f][1,2,4]triazin-4(3H)-one;
(+)-2-((3,4-trans)-1-(4-fluorobenzyl)-4-methylpyrrolidin-3-yl)-7-(4-fluorocyclohexyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4-fluorocyclohexyl)imidazol[1,5-f][1,2,4]triazin-4(3H) (+)-2-((3,4-trans)-1-benzyl-4-methylpyrrolidin-3-yl)-7-(4-fluorocyclohexyl)imidazo[1,5-f][1,2,4]triazin-4(3H)-one;
(+)-2-((3,4-trans)-1-benzyl-4-methylpyrrolidin-3-yl)-7-(4-fluorocyclohexyl)imidazo[1,5-f][1,2,4]triazin-4(3H)-one;
(+)-7-(4-fluorocyclohexyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-7-(4-fluorocyclohexyl)-2-((3,4-trans)-4-methyl-1-(pyrazin-2-ylmethyl)pyrrolidin-3-yl)imidazo[1,5-f][1,2,4]triazin-4(3H)-one;
(+)-7-(4-fluorocyclohexyl)-2-((3,4-trans)-4-methyl-1-(pyridin-2-ylmethyl)pyrrolidin-3-yl)imidazo[1,5-f][1,2,4]triazin-4(3H)-one;
(+)-7-(4-fluorocyclohexyl)-2-((3,4-trans)-4-methyl-1-(pyridin-3-ylmethyl)pyrrolidin-3-yl)imidazo[1,5-f][1,2,4]triazin-4(3H)-one;
(+)-7-(4-fluorocyclohexyl)-2-((3,4-trans)-4-methyl-1-(pyrimidin-2-ylmethyl)pyrrolidin-3-yl)imidazo[1,5-f][1,2,4]triazin-4(3H)-one; (+)-2-((3,4-trans)-1-((1H-benzo [d]imidazol-2-yl)methyl)-4-methylpyrrolidin-3-yl)-7-(4-fluorophenyl)imidazo[1,5-f][1,2,4]triazin-4(3H)-one;
(+)-2-((3,4-trans)-1-(4-chlorobenzyl)-4-methylpyrrolidin-3-yl)-7-(4-fluorophenyl)imidazo[1,5-f][1,2,4]triazin-4(3H)-one;
(+)-2-((3,4-trans)-1-(4-fluorobenzyl)-4-methylpyrrolidin-3-yl)-7-(4-fluorophenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4-fluorophenyl)imidazo[1,5-f][1,2,4]triazin-4(3H)-one;
(+)-2-((3,4-trans)-1-benzyl-4-methylpyrrolidin-3-yl)-7-(4-fluorophenyl)imidazo[1,5-f][1,2,4]triazin-4(3H)-one;
(+)-7-(4-fluorophenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-7-(4-fluorophenyl)-2-(3,4-trans)-4-methyl-1-(pyrazin-2-ylmethyl)pyrrolidin-3-yl)imidazo[1,5-f][1,2,4]triazin-4(3H)-one;
(+)-7-(4-fluorophenyl)-2-((3,4-trans)-4-methyl-1-(pyridin-2-methyl)pyrrolidin-3-yl)imidazo[1,5-f][1,2,4]triazin-4(3H)-one;
(+)-7-(4-fluorophenyl)-2-((3,4-trans)-4-methyl-1-(pyridin-3-ylmethyl)pyrrolidin-3-yl)imidazo[1,5-f][1,2,4]triazin-4(3H)-one;
(+)-7-(4-fluorophenyl)-2-((3,4-trans)-4-methyl-1-(pyrimidin-2-ylmethyl)pyrrolidin-3-yl)imidazo[1,5-f][1,2,4]triazin-4(3H)-one;
(+)-2-((3,4-trans)-1-(4(1H-benzo [d]imidazol-2-yl)methyl)-4-methylpyrrolidin-3-yl)-7-(3-fluorophenyl)imidazo[1,5-f][1,2,4]triazin-4(3H)-one;
(+)-2-((3,4-trans)-1-(4-chlorobenzyl)-4-methylpyrrolidin-3-yl)-7-(3-f uorophenyl)imidazo[1,5-f][1,2,4]triazin-4(3H)-one;
(+)-2-((3,4-trans)-1-(4-fluorobenzyl)-4-methylpyrrolidin-3-yl)-7-(3-f uorophenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(3-fluorophenyl)imidazo[1,5-f][1,2,4]triazin-4(3H)-one; (+)-2-((3,4-trans)-1-benzyl-4-methylpyrrolidin-3-yl)-7-(3-fluorophenyl)imidazo[1,5-f][1,2,4]triazin-4(3H)-one;
(+)-7-(3-fluorophenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-7-(3-fluorophenyl)-2-((3,4-trans)-4-methyl-1-(pyrazin-2-ylmethyl)pyrrolidin-3-yl)imidazo[1,5-f][1,2,4]triazin-4(3H)-one;
(+)-7-(3-fluorophenyl)-2-((3,4-trans)-4-methyl-1-(pyridin-2-ylmethyl)pyrrolidin-3-yl)imidazo[1,5-f][1,2,4]triazin-4(3H)-one;
(+)-7-(3-fluorophenyl)-2-((3,4-trans)-4-methyl-1-(pyridin-3-ylmethyl)pyrrolidin-3-yl)imidazo[1,5-f][1,2,4]triazin-4(3H)-one;
(+)-7-(3-fluorophenyl)-2-((3,4-trans)-4-methyl-1-(pyrimidin-2-ylmethyl)pyrrolidin-3-yl)imidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-o-tolylimidazo[1,5-f][1,2,4]triazin-4(3H)-one;
(+)-2-((3,4-trans)-1-(4-chlorobenzyl)-4-methylpyrrolidin-3-yl)-7-o-tolylimidazo[1,5-f][1,2,4]triazin-4(3H)-one;
(+)-2-((3,4-trans)-1-(4-fluorobenzyl)-4-methylpyrrolidin-3-yl)-7-o-tolylimidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-o-tolylimidazo[1,5-f][1,2,4]triazin-4(3H)-one;
(+)-2-((3,4-trans)-1-(imidazo[1,2-a]pyridin-2-ylmethyl)-4-methylpyrrolidin-3-yl)-7-o-tolylimidazo[1,5-f][1,2,4]triazin-4(3H)-one;
(+)-2-((3,4-trans)-1-benzyl-4-methylpyrrolidin-3-yl)-7-o-tolylimidazo[1,5-f][1,2,4]triazin-4(3H)-one;
(+)-2-((3,4-trans)-4-methyl-1-(pyrazin-2-ylmethyl)pyrrolidin-3-yl)-7-o-tolylimidazo[1,5-f][1,2,4]triazin-4(3H)-one;
(+)-2-((3,4-trans)-4-methyl-1-(pyridin-2-ylmethyl)pyrrolidin-3-yl)-7-o-tolylimidazo[1,5-f][1,2,4]triazin-4(3H)-one; (+)-2-((3,4-trans)-4-methyl-1-(pyridin-3-ylmethyl)pyrrolidin-3-yl)-7-o-tolylimidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-(4,5-difluoro-2-methylphenyl)imidazo[1,5-f][1,2,4]triazin-4(3H)-one;
(+)-2-((3,4-trans)-1-(4-chlorobenzyl)-4-methylpyrrolidin-3-yl)-7-(4,5-difluoro-2-methylphenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4,5-difluoro-2-methylphenyl)imidazo[1,5-f][1,2,4]triazin-4(3H)-one;
(+)-2-((3,4-trans)-1-benzyl-4-methylpyrrol idin-3-yl)-7-(4,5-difluoro-2-methylphenyl)imidazo[1,5-f][1,2,4]triazin-4(3H)-one;
(+)-7-(4,5-difluoro-2-methylphenyl)-2-((3,4-trans)-1-(4-fluorobenzyl)-4-methylpyrrolidin-3-yl)imidazo[1,5-f][1,2,4]triazin-4(3H)-one;
(+)-7-(4,5-difluoro-2-methylphenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-7-(4,5-difluoro-2-methylphenyl)-2-((3,4-trans)-4-methyl-1-(pyrazin-2-ylmethyl)pyrolidin-3-yl)imidazo[1,5-f][1,2,4]triazin-4(3H)-one;
(+)-7-(4,5-difluoro-2-methylphenyl)-2-((3,4-trans)-4-methyl-1-(pyridin-2-ylmethyl)pyrrolidin-3-yl)imidazo[1,5-f][1,2,4]triazin-4(3H)-one;
(+)-7-(4,5-difluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(+)-7-(4,5-difluoro-2-methylphenyl)-2-((3,4-trans)-4-methyl-1-(pyrimidin-2-ylmethyl)pyrrolidin-3-yl)imidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-phenylimidazo[1,5-f][1,2,4]triazin-4(3H)-one;
(+)-2-((3,4-trans)-1-(4-chlorobenzyl)-4-methylpyrrolidin-3-yl)-7-phenylimidazo[1,5-f]triazin-4(3H)-one;
(+)-2-((3,4-trans)-1-(4-fluorobenzyl)-4-methylpyrrolidin-3-yl)-7-phenylimidazo[1,5-f][1,2,4]triazin-4(3H)-one, (+)-2-((3,4-trans)-1-([1,2,4]triazolo[1,5-a]pyridin-2-ylmethyl)-4-methylpyrrolidin-3-yl)-7-phenylimidazo[1,5-f][1,2,4]triazin-4(3H)-one;
(+)-2-((3,4-trans)-1-(imidazo[1,2-a]pyridin-2-ylmethyl)-4-methylpyrrolidin-3-yl)-7-phenylimidazo[1,5-f][1,2,4]triazin-4(3H)-one;
(+)-2-((3,4-trans)-1-berizyl-4-methylpyrrolidin-3-yl)-7-phenylimidazo[1,5-f][1,2,4]triazin-4(3H)-one;
(+)-2-((3,4-trans)-4-methyl-1-(pyrazin-2-ylmethyl)pyrrolidin-3-yl)-7-phenylimidazo[1,5-f][1,2,4]triazin-4(3H)-one;
(+)-2-((3,4-trans)-4-methyl-1-(pyridin-2-ylmethyl)pyrrolidin-3-yl)-7-phenylimidazo[1,5-f][1,2,4]triazin-4(3H)-one;
(+)-2-((3,4-trans)-4-methyl-1-(pyridin-3-ylmethyl)pyrrolidin-3-yl)-7-phenylimidazo[1,5-f][1,2,4]triazin-4(3H)-one;
(+)-2-((3,4-trans)-4-methyl-1-(pyrimidin-2-ylmethyl)pyrrolidin-3-yl)-7-phenylimidazo[1,5-f][1,2,4]triazin-4(3H)-one;
(+)-2-((3,4-trans)-1-((1H-benzo [d]imidazol-2-yl)methy)-4-methylpyrrolidin-3-yl)-7-(4-(trifluoromethyl)phenyl)imidazo[1,5-f][1,2,4]triazin-4(3H)-one;
(+)-2-((3,4-trans)-1-(4-chlorobenzyl)-4-methylpyrrolidin-3-yl)-7-(4-(trifluoromethyl)phenyl)imidazo[1,5-f][1,2,4]triazin-4(3H)-one;
(+)-2-((3,4-trans)-1-(4-fluorobenzyl)-4-methylpyrrolidin-3-yl)-7-(4-(trifluoromethyl)phenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4-(trifluoromethyl)phenyl)imidazo[1,5-f][1,2,4]triazin-4(3H)-one;
(+)-2-((3,4-trans)-1-(imidazo[1,2-a]pyridin-2-ylmethyl)-4-methylpyrrolidin-3-yl)-7-(4-(trifluoromethyl)phenyl)imidazo[1,5-f][1,2,4]triazin-4(3H)-one;
(+)-2-((3,4-trans)-1-benzyl-4-methylpyrrolidin-3-yl)-7-(4-(trifluoromethyl)phenyl)imidazo[1,5-f][1,2,4]triazin-4(3H)-one;
(+)-2-((3,4-trans)-4-methyl-1-(pyrazin-2-ylmethyl)pyrrolidin-3-yl)-7-(4-(trifluoromethyl)phenyl)imidazo[1,5-f][1,2,4]triazin-4(3H)-one; (+)-2-((3,4-trans)-4-methyl-1-(pyridin-2-ylmethyl)pyrrolidin-3-yl)-7-(4-(trifluoromethyl)phenyl)imidazo[1,5-f][1,2,4]triazin-4(3H)-one;
(+)-2-((3,4-trans)-4-methyl-1-(pyridin-3-ylmethyl)pyrrolidin-3-yl)-7-(4-(trifluoromethyl)phenyl)imidazo[1,5-f][1,2,4]triazin-4(3H)-one;
(+)-2-((3,4-trans)-4-methyl-1-(pyrimidin-2-ylmethyl)pyrrolidin-3-yl)-7-(4-(trifluoromethyl)phenyl)imidazo[1,5-f][1,2,4]triazin-4(3H)-one;
(+)-2-((3,4-trans)-1-((1H-benzo [d]imidazol-2-yl)methyl)-4-methylpyrrolidin-3-yl)-7-(4-fluoro-2-methylphenyl)imidazo[1,5-f][1,2,4]triazin-4(3H)-one;
(+)-2-((3,4-trans)-1-(4-chlorobenzyl)-4-methylpyrrolidin-3-yl)-7-(4-fluoro-2-methylphenyl)imidazo[1,5-f][1,2,4]triazin-4(3H)-one;
(+)-2-((3,4-trans)-1-([1,2,4]triazolo[1,5-a]pyridin-2-ylmethyl)-4-methylpyrrolidin-3-yl)-7-(4-fluoro-2-methylphenyl)imidazo[1,5-f][1,2,4]triazin-4(3H)-one;
(+)-7-(4-fluoro-2-methylphenyl)-2-((3,4-trans)-1-(4-fluorobenzyl)-4-methylpyrrolidin-3-yl)imidazo[1,5-f][1,2,4]triazin-4(3H)-one;
(+)-7-(4-fluoro-2-methylphenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(+)-7-(4-fluoro-2-methylphenyl)-2-((3,4-trans)-4-methyl-1-(pyrazin-2-ylmethyl)pyrrolidin-3-yl)imidazo[1,5-f][1,2,4]triazin-4(3H)-one;
(+)-7-(4-fluoro-2-methylphenyl)-2-((3,4-trans)-4-methyl-1-(pyridin-2-ylmethyl)pyrrolidin-3-yl)imidazo[ 1,5-f][1,2,4]triazin-4(3H-one;
(+)-7-(4-fluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(+)-2-((3,4-trans)-4-methyl-1-(4-(trifluoromethyl)pyrimidin-2-yl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(2,2,2-trifluoroethyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((2-methylpyridin-3-yl)methyl)pyrrolidin-3-yl)-7-(tetrahydro-2H-pyran-4-yl)imidazo[1,5-f][1,2,4]triazin-4(3H)-one; (+)-2-((3,4-trans)-4-methyl-1-(quinolin-8-ylmethyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((3-methylpyridin-2-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((2-methylpyridin-4-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((2-propylpyrimidin-5-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(2-(pyrimidin-2-yl)ethyl)pyrrolidin-3-yl)-7-(tetrahydro-2H-pyran-4-yl)imidazo[1,5-f][1,2,4]triazin-4(3H)-one;
(+)-2-((3,4-trans)-1-((1,5-naphthyridin-4-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((1,8-naphthyridin-4-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((2-(dimethylamino)pyridin-4-yl)methyl)-4-methylpyrrolidin-3-yl)-7-(tetrahydro-2H-pyran-4-yl)imidazo[1,5-i\hat{ }[1,2,4]triazin-4(3H)-one;
(+)-2-((3,4-trans)-1-(cinnolin-3-ylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-4-methyl-1-((4,5,6,7-tetrahydropyrazolo[1,5-a]pyridin-3-yl)methyl)pyrrolidin-3-yl)-7-(teta\hat{ }4(3H)-one;
(+)-2-((3,4-trans)-4-methyl-1-(2-(pyrimidin-5-yl)ethyl)pyrrolidin-3-yl)-7-(tetrahydro-2H-pyran-4-yl)imidazo[1,5-f][1,2,4]triazin-4(3H)-one;
(+)-2-((3,4-trans)-1-((8-fluoroquinolin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(benzo[c][1,2,5]thiadiazol-5-ylmethyl)-4-methylpyrrolidin-3-yl)-7-(tetrahydro-2H-pyran-4-yl)imidazo[1,5-\hat{ }[1,2,4]triazin-4(3H)-one; (+)-2-((3,4-trans)-4-methyl-1-((1-methyl-1H-imidazol-2-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((1-methyl-1H-imidazol-5-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((1-methyl-1H-pyrazol-3-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((1-methyl-1H-pyrazol-4-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((1-methyl-1H-pyrazol-5-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(thiazol-2-ylmethyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(quinuclidin-4-ylmethyl)pyrrolidin-3-yl)-7-(tetrahydro-2H-pyran-4-yl)imidazo[1,5-f][1,2,4]triazin-4(3H)-one;
(+)-2-((3,4-trans)-1-((1,3,5-triazin-2-yl))methyl)-4-methylpyrrolidin-3-yl)-7-(tetrahydro-2H-pyran-4-yl)imidazo[1,5-f][1,2,4]triazin-4(3H)-one;
(+)-2-fluoro-5-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzonitrile;
(+)-2-((3,4-trans)-1-((4,6-dimethylpyrimidin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chloropyridin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(2,3-dichlorobenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(3-chloro-4-fluorobenzyl)-4-methylpyrrolidin-3-yl)-7-(tetrahydro-2H-pyran-4-yl)imidazo[1,5-f][1,2,4]triazin-4(3H)-one; (+)-2-((3,4-trans)-4-methyl-1-((4-methylpyridin-2-yl)methyl)pyrrolidin-3-yl)-7-(tetrahydro-2H-pyran-4-yl)imidazo[1,5-f][1,2,4]triazin-4(3H)-one; (+)-2-((3,4-trans)-4-methyl-1-((6-methylpyridin-2-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(pyrido[2,3-d]pyrimidin-2-ylmethyl)pyrrolidin-3-yl)-7-(tetrahydro-2H-pyran-4-yl)imidazo[1,5-f] [1,2,4]triazin-4(3H)-one;
(+)-2-((3,4-trans)-4-methyl-1-(pyrido [3,2-b]pyrazin-3-ylmethyl)pyrrolidin-3-yl)-7-(tetrahydro-2H-pyran-4-yl)imidazo[1,5-f][1,2,4]triazin-4(3H)-one;
(+)-tert-butyl 4-(((3,4-trans)-3-methyl-4-(4-oxo-7-(tetrahydro-2H-pyran-4-yl)-3,4-dihydroimidazo[1,5-f] [1,2,4]triazin-2-yl)pyrrolidin-1-yl)methyl)piperidine-1-carboxylate,
(+)-2-((3,4-trans)-1-((1,2,4-oxadiazol-5-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((4,4-difluorocyclohexyl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chlorofuran-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chlorofuran-3-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chlorothiophen-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chlorothiophen-3-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(cyclopentylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(cyclopropylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(furan-2-ylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(furan-3-ylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-4-methyl-1-((1-methylpiperidin-4-yl)methyl)pyrrolidin-3-yl)-7-(tetrahydro-2H-pyran-4-yl)imidazo[1, 5-f][1,2,4]triazin-4(3H)-one;
(+)-2-((3,4-trans)-4-methyl-1-((tetrahydro-2H-pyran-4-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(piperidin-4-ylmethyl)pyrrolidin-3-yl)-7-(tetrahydro-2H-pyran-4-yl)imidazo[1,5-f][1,2,4]triazin-4(3H-one;
(+)-2-((3,4-trans)-4-methyl-1-(thiophen-2-ylmethyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(thiophen-3-ylmethyl)pyrrolidin-3-yl)-7-(tetrahydro-2H-pyran-4-yl)imidazo[1,5-f][1,2,4]triazin-4(3H)-one;
(+)-2-((3,4-trans)-1-((2-methoxypyridin-4-yl)methyl)-4-methylpyrrolidin-3-yl)-7-(tetrahydro-2H-pyran-4-yl)imidazo[1,5-f][1,2,4]triazin-4(3H)-one; (+)-2-((3,4-trans)-1-((2-methoxypyrimidin-5-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((4-methoxypyridin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-chloropyridin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-fluoropyridin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-methoxypyridin-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-14(5-methoxypyridin-3-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(2-chloro-5-fluorobenzyl)-4-methylpyrrolidin-3-yl)-7-(tetrahydro-2H-pyran-4-yl)imidazo[1,5-f][1,2,4]triazin-4(3H)-one; (+)-2-((3,4-trans)-1-(4-chloro-3-(trifluoromethyl)benzyl)-4-methylpyrrolidin-3-yl)-7-(tetrahydro-2H-pyran-4-yl)imidazo[1,5-f][1,2,4]triazin-4(3H)-one; (+)-2-((3,4-trans)-1-(4-chloro-3-methylbenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(5-chloro-2-fluorobenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-4-methyl-1-((5-(trifluoromethyl)pyridin-2-yl)methyl)pyrrolidin-3-yl)-7-(teta\hat{ }

### 4(3H)-one;

(+)-2-((3,4-trans)-4-methyl-1-((6-(trifluoromethyl)pyridin-2-yl)methyl)pyrrolidin-3-yl)-7-(teta\hat{ }(3H)-one;
(+)-2-((3,4-trans)-4-methyl-1-(2-(trifluoromethoxy)benzyl)pyrrolidin-3-yl)-7-(tetrahydro-2H-pyran-4-yl)imidazo[1,5-f][1,2,4]triazin-4(3H)-one;
(+)-2-((3,4-trans)-1-((2-methoxypyridin-4-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-((5-methoxypyridin-3-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(2-chloro-3,6-difluorobenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(2-chloro-6-fluorobenzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(3-chloro-5-(trifluoromethyl)benzyl)-4-methylpyrrolidin-3-yl)-7-(tetrahydro-2H-pyran-4-yl)imidazo[1,5-f][1,2,4]triazin-4(3H)-one;
(+)-2-((3,4-trans)-4-methyl-1-((3-(trifluoromethyl)pyridin-2-yl)methyl)pyrrolidin-3-yl)-7-(tetrahydro-2H-pyran-4-yl)imidazo[1,5-f][1,2,4]triazin\hat{ }4(3H)-one;
(+)-2-((3,4-trans)-4-methyl-1-((4-(trifluoromethyl)pyridin-2-yl)methyl)pyrrolidin-3-yl)-7-(tetrahydro-2H-pyran-4-yl)imidazo[1,5-f][1,2,4]triazin-4(3H)-one; (+)-2-(3,4-trans)-4-methyl-1-((5-(trifluoromethyl)pyridin-2-yl)methyl)pyrrolidin-3-yl)-7-(tetrah\hat{ }

### 4(3H)-one;

(+)-2-((3,4-trans)-4-methyl-1-((6-(trifluoromethyl)pyridin-2-yl)methyl)pyrrolidin-3-yl)-7-(tetra\hat{ }(3H)-one;
(+)—N,N-dimethyl-2-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(+)—N,N-dimethyl-3-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(+)—N,N-dimethyl-4-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(+)-2-((3,4-trans)-1-((1,2,4-oxadiazol-3-yl)methyl)-4-methylpyrrolidin-3-yl)-7-(tetrahydro-2H-pyran-4-yl)imidazo[1,5-][1,2,4]triazin-4(3H)-one;
(+)-2-((3,4-trans)-1-((1,3,4-oxadiazol-2-yl)methyl)-4-methylpyrrolidin-3-yl)-7-(tetrahydro-2H-pyran-4-yl)imidazo[1,5-f][1,2,4]triazin-4(3H)-one;
(+)-2-((3,4-trans)-1-(biphenyl-4-ylmethyl)-4-methylpyrrolidin-3-yl)-7-(tetrahydro-2H-pyran-4-yl)imidazo[1,5-f][1,2,4]triazin-4(3H)-one;
(+)-2-((3,4-trans)-4-methyl-1-1-methyl-1H-1,2,4-triazol-3-yl)methyl)pyrrolidin-3-yl)-7-(tetrah\hat{ }4(3H)-one;
(+)-2-((3,4-trans)-4-methyl-1-((1-methyl-1H-imidazol-4-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((2-methylthiazol-4-yl)methyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-((5-(trifluoromethyl)-1,2,4-oxadiazol-3-yl)methyl)pyrrolidin-3-yl)-7-(tetrahydro-2H-pyran-4-yl)imidazo[1,5-f][1,2,4]triazin-4(3H)-one, (+)-2-((3,4-trans)-4-methyl-1-((5-methyl-1,2,4-oxadiazol-3-yl)methyl)pyrrolidin-3-yl)-7-(te\hat{ }\hat{ }(3H)-one;
(+)-2-((3,4-trans)-4-methyl-1-((5-methyl-1H-1,2,4-triazol-3-yl)methyl)pyrrolidin-3-yl)-7-(te\hat{ }\hat{ }(3H)-one;
(+)-2-((3,4-trans)-4-methyl-1-(4-(pyridin-4-yl)benzyl)pyrrolidin-3-yl)-7-(tetrahydro-2H-pyran-4-yl)imidazo[1,5-f][1,2,4]triazin-4(3H)-one;
(+)-2-((3,4-trans)-4-methyl-1-(oxazol-2-ylmethyl)pyrrolidin-3-yl)-7-(tetrahydro-2H-pyran-4-yl)imidazo[1,5-f][1,2,4]triazin-4(3H)-one;
(−)-7-(4,4-difluorocyclohexyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-2-((3,4-trans)-1-((3aH-benzo[d]imidazol-2-yl)methyl-)-4-methylpyrrolidin-3-yl)-7-(4,4-difluorocyclohexyl)imidazo[1,5-f][1,2,4]triazin-4(3H)-one;
(−)-7-(4,4-difluorocyclohexyl)-2-((3,4-trans)-4-methyl-1-(pyridin-2-ylmethyl)pyrrolidin-3-yl)imidazo[1,5-f][1,2,4]triazin-4(3H)-one;
(−)-7-(4,4-difluorocyclohexyl)-2-((3,4-trans)-4-methyl-1-(pyridin-3-ylmethyl)pyrrolidin-3-yl)imidazo[1,5-f][1,2,4]triazin-4(3H)-one;
(−)-7-(4,4-difluorocyclohexyl)-2-((3,4-trans)-4-methyl-1-(pyrazin-2-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(5-fluoro-2-methylphenyl)imidazo[1,5-f][1,2,4]triazin-4(3H)-one;
(−)-2-((3,4-trans)-1-(4-chlorobenzyl)-4-methylpyrrolidin-3-yl)-7-(5-fluoro-2-methylphenyl)imidazo[1,5-f][1,2,4]triazin-4(3-H)-one;
(−)-2-((3,4-trans)-1-([1,2,4]triazolo[1,5-a]pyridin-2-ylmethyl)-4-methylpyrrolidin-3-yl)-7-(5-fluoro-2-methylphenyl)imidazo[1,5-i\hat{ }[1,2,4]M
4(3H)-one;
(−)-7-(5-fluoro-2-methylphenyl)-2-((3,4-trans)-1-(4-fluorobenzyl)-4-methylpyrrolidin-3-yl)imidazo[1,5-f][1,2,4]triazin-4(3H)-one; (−)-7-(5-fluoro-2-methylphenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(5-fluoro-2-methylphenyl)-2-((3,4-trans)-4-methyl-1-(pyrazin-2-ylmethyl)pyrrolidin-3-yl)imidazo[1,5-f][1,2,4]triazin-4(3H)-one;
(−)-7-(5-fluoro-2-methylphenyl)-2-((3,4-trans)-4-methyl-1-(pyridin-2-ylmethyl)pyrrolidin-3-yl)imidazo[1,5-f][1,2,4]triazin-4(3H)-one;
(−)-(−)-7-(5-fluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(4-fluorocyclohexyl)imidazo[1,5-f][1,2,4]triazin-4(3H)-one;
(−)-2-((3,4-trans)-1-(4-chlorobenzyl)-4-methylpyrrolidin-3-yl)-7-(4-fluorocyclohexyl)imidazo[1,5-f][1,2,4]triazin-4(3H)-one;
(−)-2-((3,4-trans)-1-(4-fluorobenzyl)-4-methylpyrrolidin-3-yl)-7-(4-fluorocyclohexyl)imidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-(4-fluorocy\hat{ }\hat{ }(−)-24 (3,4-trans)-1-benzyl-4-methylpyrrolidin-3-yl)-7-(4-fluorocyclohexyl)imidazo[1,5-f][1,2,4]triazin-4(3H)-one;
(−)-2-((3,4-trans)-1-benzyl-4-methylpyrrolidin-3-yl)-7-(4-fluorocyclohexyl)imidazo[1,5-f][1,2,4]triazin-4(3H)-one;
(−)-7-(4-fluorocyclohexyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(4-fluorocyclohexyl)-2-((3,4-trans)-4-methyl-1-(pyrazin-2-ylmethyl)pyrrolidin-3-yl)imidazo[1,5-f][1,2,4]triazin-4(3H)-one;
(−)-7-(4-fluorocyclohexyl)-2-((3,4-trans)-4-methyl-1-(pyridin-2-ylmethyl)pyrrolidin-3-yl)imidazo[1,5-f][1,2,4]triazin-4(3H)-one;
(−)-7-(4-fluorocyclohexyl)-2-((3,4-trans)-4-methyl-1-(pyridin-3-ylmethyl)pyrrolidin-3-yl)imidazo[1,5-f][1,2,4]triazin-4(3H)-one; (−)-7-(4-fluorocyclohexyl)-2-((3,4-trans)-4-methyl-1-(pyrimidin-2-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(4-fluorophenyl)imidazo[1,5-f][1,2,4]triazin-4(3H)-one;
(−)-2-((3,4-trans)-1-(4-chlorobenzyl)-4-methylpyrrolidin-3-yl)-7-(4-fluorophenyl)imidazo[1,5-f][1,2,4]triazin-4(3H)-one;
(−)-2-((3,4-trans)-1-(4-fluorobenzyl)-4-methylpyrrolidin-3-yl)-7-(4-fluorophenyl)imidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-(4-fluoro\hat{ }
(−)-2-((3,4-trans)-1-benzyl-4-methylpyrrolidin-3-yl)-7-(4-fluorophenyl)imidazo[1,5-f][1,2,4]triazin-4(3H)-one;
(−)-7-(4-fluorophenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(4-fluorophenyl)-2-((3,4-trans)-4-methyl-1-(pyrazin-2-ylmethyl)pyrrolidin-3-yl)imidazo[1,5-f][1,2,4]triazin-4(3H)-one;
(−)-7-(4-fluorophenyl)-2-((3,4-trans)-4-methyl-1-(pyridin-2-ylmethyl)pyrrolidin-3-yl)imidazo[1,5-f][1,2,4]triazin-4(3H)-one;
(−)-7-(4-fluorophenyl)-2-((3,4-trans)-4-methyl-1-(pyridin-3-ylmethyl)pyrrolidin-3-yl)imidazo[1,5-f][1,2,4]triazin-4(3H)-one;
(−)-7-(4-fluorophenyl)-2-((3,4-trans)-4-methy, 1-1-(pyrimidin-2-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(3-fluorophenyl)imidazo[1,5-f][1,2,4]triazin-4(3H)-one;
(−)-2-((3,4-trans)-1-(4-chlorobenzyl)-4-methylpyrrolidin-3-yl)-7-(3-fluorophenyl)imidazo[1,5-f][1,2,4]triazin-4(3H)-one;
(−)-2-(3,4-trans)-1-(4-fluorobenzyl)-4-methylpyrrolidin-3-yl)-7-(3-f uorophenyl)imidazo[1,5-f] [1,2,4]triazin-4(3H)-one; (−)-2-((3,4-trans)-1-([1,2,4]
   triazolo[1,5-a]pyri
   methylpyrrolidin-3-yl)-7-(3-fluorophenyl)imidazo[1,5-f][1,2,4]triazin-4(3H)-one;
(−)-2-((3,4-trans)-1-benzyl-4-methylpyrrolidin-3-yl)-7-(3-fluorophenyl)imidazo)[1,5-f][1,2,4]triazin-4(3H)-one;
(−)-7-(3-fluorophenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(3-fluorophenyl)-2-((3,4-trans)-4-methyl-1-(pyrazin-2-ylmethyl)pyrrolidin-3-yl)imidazo[1,5-f][1,2,4]triazin-4(3H)-one;
(−)-7-(3-fluorophenyl)-2-((3,4-trans)-4-methyl-1-(pyridin-2-ylmethyl)pyrrolidin-3-yl)imidazo[1,5-f][1,2,4]triazin-4(3H)-one;
(−)-7-(3-fluorophenyl)-2-((3,4-trans)-4-methyl-1-(pyridin-3-ylmethyl)pyrrolidin-3-yl)imidazo[1,5-f][1,2,4]triazin-4(3H)-one;
(−)-7-(3-fluorophenyl)-2-((3,4-trans)-4-methyl-1-(pyrimidin-2-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-o-tolylimidazo[1,5-f][1,2,4]triazin-4(3H)-one;
(−)-2-((3,4-trans)-1-(4-chlorobenzyl)-4-methylpyrrolidin-3-yl)-7-o-tolylimidazo[1,5-f][1,2,4]triazin-4(3H)-one;
(−)-2-((3,4-trans)-1-(4-fluorobenzyl)-4-methylpyrrolidin-3-yl)-7-o-tolylimidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-o-tolylimidazo[1,5-f][1,2,4]triazin-4(3H)-one;
(−)-2-((3,4-trans)-1-(imidazo[1,2-a]pyridin-2-ylmethyl)-4-methylpyrrolidin-3-yl)-7-o-tolylimidazo[1,5-f][1,2,4]triazin-4(3H)-one;
(−)-2-((3,4-trans)-1-benzyl-4-methylpyrrolidin-3-yl)-7-o-tolylimidazo[1,5-f][1,2,4]triazin-4(3H)-one;
(−)-2-((3,4-trans)-4-methyl-1-(pyrazin-2-ylmethyl)pyrrolidin-3-yl)-7-o-tolylimidazo[1,5-f][1,2,4]triazin-4(3H)-one; (−)-2-((3,4-trans)-4-methyl-1-(pyridin-2-ylmethyl)pyrrolidin-3-yl)-7-o-tolylimidazo[1,5-f][1,2,4]triazin-4(3H)-one;
(−)-2-((3,4-trans)-4-methyl-1-(pyridin-3-ylmethyl)pyrrolidin-3-yl)-7-o-tolylimidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(4,5-difluoro-2-methylphenyl)imidazo[1,5-i\hat{ }[1,2,4]triazin-4(3H)-one;
(−)-2-((3,4-trans)-1-(4-chlorobenzyl)-4-methylpyrrolidin-3-yl)-7-(4,5-difluoro-2-methylphenyl)imidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-(4,5-difluoro-2-methylphenyl)imidazo[1,5-f][1,2,4]triazin-4(3H)-one;
(−)-2-((3,4-trans)-1-benzyl-4-methylpyrrolidin-3-yl)-7-(4,5-difluoro-2-methylphenyl)imidazo[1,5-f][1,2,4]triazin-4(3H)-one;
(−)-7-(4,5-difluoro-2-methylphenyl)-2-((3,4-trans)-1-(4-fluorobenzyl)-4-methylpyrrolidin-3-yl)imidazo[1,5-f][1,2,4]triazin-4(3H)-one;
(−)-7-(4,5-difluoro-2-methylphenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(4,5-difluoro-2-methylphenyl)-2-((3,4-trans)-4-methyl-1-(pyrazin-2-ylmethyl)pyrrolidin-3-yl)imidazo[1,5-f][1,2,4]triazin-4(3H)-one;
(−)-7-(4,5-difluoro-2-methylphenyl)-2-((3,4-trans)-4-methyl-1-(pyridin-2-ylmethyl)pyrrolidin-3-yl)imidazo[1,5-f][1,2,4]triazin-4(3H)-one;
(−)-7-(4,5-difluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(−)-7-(4,5-difluoro-2-methylphenyl)-2-((3,4-trans)-4-methyl-1-(pyrimidin-2-ylmethyl)pyrrolidin-3-yl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-phenylimidazo[1,5-f][1,2,4]triazin-4(3H)-one;
(−)-2-((3,4-trans)-1-(4-chlorobenzyl)-4-methylpyrrolidin-3-yl)-7-phenylimidazo[1,5-f][1,2,4]triazin-4(3H)-one; (−)-2-((3,4-trans)-1-(4-fluorobenzyl)-4-methylpyrrolidin-3-yl)-7-phenylimidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-phenylimidazo[1,5-f][1,2,4]triazin-4(3H)-one;
(−)-2-((3,4-trans)-1-(imidazo[1,2-a]pyridin-2-ylmethyl)-4-methylpyrrolidin-3-yl)-7-phenylimidazo[1,5-f] [1,2,4]triazin-4(3H)-one;
(−)-2-((3,4-trans)-1-benzyl-4-methylpyrrolidin-3-yl)-7-phenylimidazo[1,5-f][1,2,4]triazin-4(3H)-one;
(−)-2-((3,4-trans)-4-methyl-1-(pyrazin-2-ylmethyl)pyrrolidin-3-yl)-7-phenylimidazo[1,5-f][1,2,4]triazin-4(3H)-one;
(−)-2-((3,4-trans)-4-methyl-1-(pyridin-2-ylmethyl)pyrrolidin-3-yl)-7-phenylimidazo[1, 5-f][1,2,4]triazin-4(3H)-one;
(−)-2-((3,4-trans)-4-methyl-1-(pyridin-3-ylmethyl)pyrrolidin-3-yl)-7-phenylimidazo[1,5-f][1,2,4]triazin-4(3H)-one;
(−)-2-((3,4-trans)-4-methyl-1-(pyrimidin-2-ylmethyl)pyrrolidin-3-yl)-7-phenylimidazo[1,5-f][1,2,4]triazin-4(3H)-one;
(−)-2-((3,4-trans)-1-((1H-benzo [d]imidazol-2-yl)methyl)-4-methylpyrrolidin-3-yl)-7-(4-(trifluoromethyl)phenyl)imidazo[1,5-f][1,2,4]triazin-4(3H)-one;
(−)-2-((3,4-trans)-1-(4-chlorobenzyl)-4-methylpyrrolidin-3-yl)-7-(4-(trifluoromethyl)phenyl)imidazo[1,5-f][1,2,4]triazin-4(3H)-one
(−)-2-((3,4-trans)-1-(4-fluorobenzyl)-4-methylpyrrolidin-3-yl)-7-(4-(trifluoromethyl)phenyl)imidazo[1,5-f][1,2,4]triazin-4(3H)-one;
(−)-2-((3,4-trans)-1-([1,2,4]triazolo[1,5-a]pyridin-2-ylmethyl)-4-methylpyrrolidin-3-yl)-7-(4-(trifluoromethyl)phenyl)imidazo[1,5-f][1,2,4]triazin-4(3H)-one;
(−)-2-((3,4-trans)-1-(imidazo[1,2-a]pyridin-2-ylmethyl)-4-methylpyrrolidin-3-yl)-7-(4-(trifluoromethyl)phenyl)imidazo[1,5-f][1,2,4]triazin-4(3H)-one;
(−)-2-((3,4-trans)-1-benzyl-4-methylpyrrolidin-3-yl)-7-(4-(trifluoromethyl)phenyl)imidazo[1,5-f][1,2,4]triazin-4(3H)-one; (−)-2-((3,4-trans)-4-methyl-1-(pyrazin-2-ylmethyl)pyrrolidin-3-yl)-7-(4-(trifluoromethyl)phenyl)imidazo[1,5-f][1,2,4]triazin-4(3H)-one;
(−)-2-((3,4-trans)-4-methyl-1-(pyridin-2-ylmethyl)pyrrolidin-3-yl)-7-(4-(trifluoromethyl)phenyl)imidazo[1,5-f][1,2,4]triazin-4(3H)-one;
(−)-2-((3,4-trans)-4-methyl-1-(pyridin-3-ylmethyl)pyrrolidin-3-yl)-7-(4-(trifluoromethyl)phenyl)imidazo[1,5-f][1,2,4]triazin-4(3H)-one;
(−)-2-((3,4-trans)-4-methyl-1-(pyrimidin-2-ylmethyl)pyrrolidin-3-yl)-7-(4-(trifluoromethyl)phenyl)imidazo[1,5-f][1,2,4]triazin-4(3H)-one;
(−)-2-((3,4-trans)-1-((1H-benzo[d]imidazol-2-yl)methyl)-4-methylpyrrolidin-3-yl)-7-(4-fluoro-2-methylphenyl)imidazo[1,5-f][1,2,4]triazin-4(3H)-one;
(−)-2-((3,4-trans)-1-(4-chlorobenzyl)-4-methylpyrrolidin-3-yl)-7-(4-fluoro-2-methylphenyl)imidazo[1,5-f][1,2,4]triazin-4(3H)-one;
(−)-2-((3,4-trans)-1-([1,2,4]triazolo[1,5-a]pyri
methylpyrrolidin-3-yl)-7-(4-fluoro\hat{ }
4(3H)-one;
(−)-7-(4-fluoro-2-methylphenyl)-2-((3,4-trans)-1-(4-fluorobenzyl)-4-methylpyrrolidin-3-yl)imidazo[1,5-] [1,2,4]triazin-4(3H)-one;
(−)-7-(4-fluoro-2-methylphenyl)-2-((3,4-trans)-1-(imidazo[1,2-a]pyridin-2-ylmethyl)-4-methylpyrrolidin-3-yl)imidazo[1,5-f][1,2,4]triazin-4(3H)-one;
(−)-7-(4-fluoro-2-methylphenyl)-2-((3,4-trans)-4-methyl-1-(pyrazin-2-ylmethyl)pyrrolidin-3-yl)imidazo[1,5-f][1,2,4]triazin-4(3H)-one;
(−)-7-(4-fluoro-2-methylphenyl)-2-((3,4-trans)-4-methyl-1-(pyridin-2-ylmethyl)pyrrolidin-3-yl)imidazo[1,5-f][1,2,4]triazin-4(3H)-one;
(−)-7-(4-fluoro-2-methylphenyl)-2-((3,4-trans)-4-methyl-1-(pyridin-3-ylmethyl)pyrrolidin-3-yl)imidazo[1,5-f][1,2,4]triazin-4(3H)-one;
(−)-2-((3,4-trans)-4-methyl-1-(4-(trifluoromethyl)pyrimidin-2-yl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(2,2,2-trifluoroethyl)pyrrolidin-3-yl)-7-(tetrahydro-2H-pyran-4-yl)imidazo[1,5-f][1,2,4]triazin-4(3H)-one; (−)-2-((3,4-trans)-4-methyl-1-((2-methylpyridin-3-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(quinolin-8-ylmethyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((3-methylpyridin-2-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((2-methylpyridin-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((2-propylpyrimidin-5-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(2-(pyrimidin-2-yl)ethyl)pyrrolidin-3-yl)-7-(tetrahydro-2H-pyran-4-yl)imidazo[1,5-f][1,2,4]triazin-4(3H)-one;
(−)-2-((3,4-trans)-1-((1,5-naphthyridin-4-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((1, 8-naphthyridin-4-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((2-(dimethylamino)pyrimidin-4-yl)methyl)-4-methylpyrrolidin-3-yl)-7-(tetrahydro-2H-pyran-4-yl)imidazo[1,5-i\hat{ }[1,2,4]triazin-4(3H)-one;
(−)-2-((3,4-trans)-1-(cinnolin-3-ylmethyl)-4-methylpyrrolidin-3-yl)-7-(tetrahydro-2H-pyran-4-yl)imidazo[1,5-f][1,2,4]triazin-4(3H)-one;
(−)-2-((3,4-trans)-4-methyl-1-((4,5,6,7-tetrahydropyrazolo[1,5-a]pyridin-3-yl)methyl)pyrrolidin-3-yl)-7-(teta\hat{ }
4(3H)-one;
(−)-2-((3,4-trans)-4-methyl-1-(2-(pyrimidin-5-yl)ethyl)pyrrolidin-3-yl)-7-(tetrahydro-2H-pyran-4-yl)imidazo[1,5-f][1,2,4]triazin-4(3H)-one;
(−)-2-((3,4-trans)-1-((8-fluoroquinolin-2-yl)methyl)-4-methylpyrrolidin-3-yl)-7-(tetrahydro-2H-pyran-4-yl)imidazo[1,5-f][1,2,4]triazin-4(3H)-one; (−)-2-((3,4-trans)-1-(benzo[c][1,2,5]thiadiazol-5-ylmethyl)-4-methylpyrrolidin-3-yl)-7-(tetrahydro-2H-pyran-4-yl)imidazo[1,5-i\hat{ }[1,2,4]triazin-4(3H)-one;
(−)-2-((3,4-trans)-4-methyl-1-((1-methyl-1H-imidazol-2-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((1-methyl-1H-imidazol-5-yl)methyl)pyrrolidin-3-yl)-7-(tetrahydro-2H-1-pyran-4-yl)imidazo[1,5-f][1,2,4]triazin-4(3H)-one;
(−)-2-((3,4-trans)-4-methyl-1-((1-methyl-1H-pyrazol-3-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((1-methyl-1H-pyrazol-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((1-methyl-1H-pyrazol-5-yl)methyl)pyrrolidin-3-yl)-7-(tetrahydro-2H-pyran-4-yl)imidazo[1,5-f][1,2,4]triazin-4(3H)-one;
(−)-2-(3,4-trans)-4-methyl-1-(thiazol-2-ylmethyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(quinuclidin-4-ylmethyl)pyrrolidin-3-yl)-7-(tetrahydro-2H-pyran-4-yl)imidazo[1,5-f][1,2,4]triazin-4(3H)-one;
(−)-2-((3,4-trans)-1-((1,3,5-triazin-2-yl)methyl)-4-methylpyrrolidin-3-yl)-7-(tetrahydro-2H-pyran-4-yl)imidazo[1,5-f][1,2,4]triazin-4(3H)-one;
(−)-2-fluoro-5-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzonitrile;
(−)-2-((3,4-trans)-1-((4,6-dimethylpyrimidin-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chloropyrimidin-2-yl)methyl)-4-methylpyrrolidin-3-yl)-7-(tetrahydro-2H-pyran-4-yl)imidazo[1,5-f][1,2,4]triazin-4(3-H)-one;
(−)-2-((3,4-trans)-1-(2,3-dichlorobenzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(3-chloro-4-fluorobenzyl)-4-methylpyrrolidin-3-yl)-7-(tetrahydro-2H-pyran-4-yl)imidazo[1,5-f][1,2,4]triazin-4(3H)-one; (−)-2-((3,4-trans)-4-methyl-1-((4-methylpyridin-2-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((6-methylpyridin-2-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(pyrido[2,3-d]pyrimidin-2-ylmethyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(pyrido[3,2-b]pyrazin-3-ylmethyl)pyrrolidin-3-yl)-7-(tetrahydro-2H-pyran-4-yl)imidazo[1,5-f][1,2,4]triazin-4(3H)-one;
(−)-tert-butyl 4-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)piperidine-1-carboxylate;
(−)-2-((3,4-trans)-1-((1,2,4-oxadiazol-5-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((4,4-difluorocyclohexyl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chlorofuran-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chlorofuran-3-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chlorothiophen-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chlorothiophen-3-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(cyclopentylmethyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(cyclopropylmethyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(furan-2-ylmethyl)-4-methylpyrrolidin-3-yl)-7-(tetrahydro-2H-pyran-4-yl)imidazo[1,5-f][1,2,4]triazin-4(3-H)-one; (−)-2-((3,4-trans)-1-(furan-3-ylmethyl)-4-methylpyrrolidin-3-yl)-7-(tetrahydro-2H-pyran-4-yl)imidazo[1,5-f][1,2,4]triazin-4(3H)-one;
(−)-2-((3,4-trans)-4-methyl-1-((1-methylpiperidin-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((tetrahydro-2H-pyran-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(piperidin-4-ylmethyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(thiophen-2-ylmethyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-(thiophen-3-ylmethyl)pyrrolidin-3-yl)-7-(tetrahydro-2H-pyran-4-yl)imidazo[1,5-f][1,2,4]triazin-4(3H)-one;
(−)-2-((3,4-trans)-1-((2-methoxypyridin-4-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((2-methoxypyrimidin-5-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((4-methoxypyridin-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-chloropyridin-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-fluoropyridin-2-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-methoxy pyridin-2-yl)methy)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-methoxypyridin-3-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(2-chloro-5-fluorobenzyl)-4-methylpyrrolidin-3-yl)-7-(tetrahydro-2H-pyran-4-yl)imidazo[1,5-f][1,2,4]triazin-4(3H)-one; (−)-2-((3,4-trans)-1-(4-chloro-3-(trifluoromethyl)benzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(4-chloro-3-methylbenzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(5-chloro-2-fluorobenzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-4-methyl-1-((5-(trifluorom yl)methyl)pyrrolidin-3-yl)-7-(teta\hat{ }

### 4(3H)-one;

(−)-2-((3,4-trans)-4-methyl-1-((6-(trifluoromethyl)pyridin-2-yl)methyl)pyrrolidin-3-yl)-7-(teta\hat{ }(3H)-one;
(−)-2-((3,4-trans)-4-methyl-1-(2-(trifluoromethoxy)benzyl)pyrrolidin-3-yl)-7-(tetrahydro-2H-pyran-4-yl)imidazo[1,5-f][1,2,4]triazin-4(3H)-one;
(−)-2-((3,4-trans)-1-((2-methoxypyridin-4-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((5-methoxypyridin-3-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(2-chloro-3,6-difluorobenzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(2-chloro-6-fluorobenzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-(3-chloro-5-(trifluoromethyl)benzyl)-4-methylpyrrolidin-3-yl)-7-(tetrahydro-2H-pyran-4-yl)imidazo[1,5-f][1,2,4]triazin-4(3H)-one;
(−)-2-((3,4-trans)-4-methyl-1-((3-(trifluoromethyl)pyridin-2-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((4-(trifluoromethyl)pyridin-2-yl)methyl)pyrrolidin-3-yl)-7-(tetrahydro-2H-pyran-4-yl)imidazo[1,5-f][1,2,4]triazin-4(3H)-one; (−)-2-((3,4-trans)-4-methyl-1-((5-(trifluoromethyl)pyridin-2-yl)methyl)pyrrolidin-3-yl)-7-(tetrah\hat{ }

### 4(3H)-one;

(−)-2-((3,4-trans)-4-methyl-1-((6-(trifluoromethyl)pyridin-2-yl)methyl)pyrrolidin-3-yl)-7-(tetrah\hat{ }(3H)-one;
(−)-N,N-dimethyl-2-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(−)-N,N-dimethyl-3-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(−)-N,N-dimethyl-4-(((3,4-trans)-3-methyl-4-(4-oxo-7-(tetrahydro-2H-pyran-4-yl)-3,4-dihydroimidazo[1,5-f][1,2,4]triazin-2-yl)pyrrolidin-1-yl)methyl)benzamide;
(−)-2-((3,4-trans)-1-((1,2,4-oxadiazol-3-yl)methyl)-4-methylpyrrolidin-3-yl)-7-(tetrahydro-2H-pyran-4-yl)imidazo[1,5-f][1,2,4]triazin-4(3H)-one;
(−)-2-((3,4-trans)-1-((1,3,4-oxadiazol-2-yl)methyl)-4-methylpyrrolidin-3-yl)-7-(tetrahydro-2H-pyran-4-yl)imidazo[1,5-][1,2,4]triazin-4(3H)-one;
(−)-2-((3,4-trans)-1-(biphenyl-4-ylmethyl)-4-methylpyrrolidin-3-yl)-7-(tetrahydro-2H-pyran-4-yl)imidazo[1,5-f][1,2,4]triazin-4(3H)-one;
(−)-2-((3,4-trans)-4-methyl-1-((1-methyl-1H-1,2,4-triazol-3-yl)methyl)pyrrolidin-3-yl)-7-(tetrah\hat{ }(3H)-one;
(−)-2-((3,4-trans)-4-methyl-1-((1-methyl-1H-imidazol-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((2-methylthiazol-4-yl)methyl)pyrrolidin-3-yl)-7-(tetrahydro-2H-pyran-4-yl)imidazo[1,5-f][1,2,4]triazin-4(3H)-one;
(−)-2-((3,4-trans)-4-methyl-1-((5-(trifluoromethyl)-1,2,4-oxadiazol-3-yl)methyl)pyrrolidin-3-yl)-7-(tetrahydro-2H-pyran-4-yl)imidazo[1,5-f][1,2,4]triazin-4(3H)-one; (−)-2-((3,4-trans)-4-methyl-1-((5-methyl-1,2,4-oxadiazol-3-yl)methyl)pyrrolidin-3-yl)-7-(tetrah\hat{ }
4(3H)-one;
(−)-2-((3,4-trans)-4-methyl-1-((5-methyl-1H-1,2,4-triazol-3-yl)methyl)pyrrolidin-3-yl)-7-(tetrahydro-2H-pyran-4-yl)imidazo[1,5-f][1,2,4]triazin-4(3H)-one;
(−)-2-(3,4-trans)-4-methyl-1-(4-(pyridin-4-yl)benzyl)pyrrolidin-3-yl)-7-(tetrahydro-2H-pyran-4-yl)imidazo[1,5-f][1,2,4]triazin-4(3H)-one; and
(−)-2-((3,4-trans)-4-methyl-1-(oxazol-2-ylmethyl)pyrrolidin-3-yl)-7-(tetrahydro-2H-pyran-4-yl)imidazo[1,5-f][1,2,4]triazin-4(3H)-one; or a pharmaceutically acceptable salt thereof.

In some cases, as disclosed in WO2012040230, PDE9 inhibitor is represented by a compound of formula (AA-II),

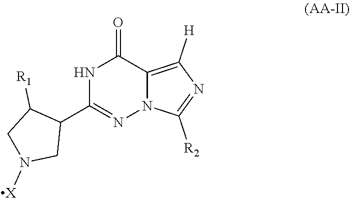

or a pharmaceutically acceptable salt thereof,
wherein:
   X is selected from a bond, C(O) or S(O)₂;
   R₁ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and heterocycloalkyloxy, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl];
   R² is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl]; and R³ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl].

In some embodiments, PDE9 inhibitor is selected from the group consisting of:
(−)-2-((3,4-trans)-1-benzyl-4-methylpyrrolidin-3-yl)-7-(tetrahydro-2H-pyran-4-yl)imidazo[1,5-fJ [1,2,4]triazin-4(3H)-one
(+)-2-((3,4-trans)-1-benzyl-4-methylpyrrolidin-3-yl)-7-(tetrahydro-2H-pyran-4-yl)imidazo[1,5fJ [1,2,4]triazin-4(3H)-one
(+)-2-((3,4-trans)-1-(imidazo[1,2-b]pyridazin-2-ylmethyl)-4-methylpyrrolidin-3-yl)-7-(tetrahydro-2H-pyran-4-yl)imidazo[5, 1-f] [1,2,4]triazin-4(3H)-one
(+)-2-((3,4-trans)-1-(imidazo[1,2-b]pyridazin-6-ylmethyl)-4-methylpyrrolidin-3-yl)-7-(tetrahydro-2H-pyran-4-yl)imidazo[5,1-fj [1,2,4]triazin-4(3H)-one
(−)-2-((3,4-trans)-1-(3-fluorobenzyl)-4-methylpyrrolidin-3-yl)₇-(tetrahydro-2H-pyran-4-yl)imidazo[5, 1-f][1,2,4]triazin-4(3H)-one
(+)-2-((3,4-trans)-1-(4-fluorobenzyl)-4-methylpyrrolidin-3-yl)-7-(tetrahydro-2H-pyran-4-yl)imidazo[5,1-fj [1,2,4]triazin-4(3H)-one
(+)-3-(((3,4-trans)-3-methyl-4-(4-oxo-7-(tetrahydro-2H-pyran-4-yl)-3,4-dihydroimidazo [5, 1-f][1,2,4]triazin-2-yl)pyrrolidin-1-yl)methyl)benzonitrile
(−)-4-(((3,4-trans)-3-methyl-4-(4-oxo-7-(tetrahydro-2H-pyran-4-yl)-3,4-dihydroimidazo [5, 1-f] [1,2,4]triazin-2-yl)pyrrolidin-1-yl)methyl)benzonitrile
(+)-2-((3,4-trans)-1-(2-fluorobenzyl)-4-methylpyrrolidin-3-yl)-7-(tetrahydro-2H-pyran-4-yl)imidazo[5, 1-fj [1,2,4]triazin-4(3H)-one (+)-2-(((3,4-trans)-3-methyl-4-(4-oxo-7-(tetrahydro-2H-pyran-4-yl)-3,4-dihydroimidazo [5,1-f]1,2,4]triazin-2-yl)pyrrolidin-1-yl)methyl)benzonitrile
(+)-2-((3,4-trans)-4-methyl-1-(pyridin-2-ylmethyl)pyrrolidin-3-yl)-7-(tetrahydro-2H-pyran-4-yl)imidazo[5, 1-f][1,2,4]triazin-4(3H)-one
(+)-2-((3,4-trans)-4-methyl-1-(quinolin-2-ylmethyl)pyrrolidin-3-yl)-7-(tetrahydro-2H-pyran-4-yl)imidazo[5, 1-f][1,2,4]triazin-4(3H)-one
(−)-2-((3,4-trans)-4-methyl-1-(quinazolin-2-ylmethyl)pyrrolidin-3-yl)-7-(tetrahydro-2H-pyran-4-yl)imidazo[5,1-f][1,2,4]triazin-4(3H)-one
(−)-2-((3,4-trans)-4-methyl-1-(quinolin-2-ylmethyl)pyrrolidin-3-yl)-7-(tetrahydro-2H-pyran-4-yl)imidazo[5, 1-f][1,2,4]triazin-4(3H)-one
(+)-7-((1 s,3R)-adamantan-1-yl)-2-((3,4-trans)-1-benzyl-4-methylpyrrolidin-3-yl)imidazo[5, 1-f][1,2,4]triazin-4(3H)-one
(−)-7-((1 s,3R)-adamantan-1-yl)-2-((3,4-trans)-1-benzyl-4-methylpyrrolidin-3-yl)imidazo[5,1-f] [1,2,4]triazin-4(3H)-one
(−)-2-((3,4-trans)-1-benzyl-4-methylpyrrolidin-3-yl)-7-(5-chloro-2-methylphenyl)imidazo[5, 1-f] [1,2,4]triazin-4(3H)-one
(+)-2-((3,4-trans)-1-benzyl-4-methylpyrrolidin-3-yl)-7-(5-chloro-2-methylphenyl)imidazo[5, 1-f] [1,2,4]triazin-4(3H)-one
(+)-2-((3,4-trans)-1-benzyl-4-methylpyrrolidin-3-yl)-7-(4-fluoro-2-methylphenyl)imidazo[5, 1-f][1,2,4]triazin-4(3H)-one (−)-2-((3,4-trans)-1-benzyl-4-methylpyrrolidin-3-yl)-7-(4-fluoro-2-methylphenyl)imidazo[5, 1-f][1,2,4]triazin-4(3H)-one
(+)-2-((3,4-trans)-1-benzyl-4-methylpyrrolidin-3-yl)-7-(4,4-difluorocyclohexyl)imidazo[5, 1-f][1,2,4]triazin-4(3H)-one
(−)-2-((3,4-trans)-1-benzyl-4-methylpyrrolidin-3-yl)-7-(4,4-difluorocyclohexyl)imidazo[5, 1-f][1,2,4]triazin-4(3H)-one
(−)-2-((3,4-trans)-4-methyl-1-(quinazolin-2-ylmethyl)pyrrolidin-3-yl)-7-(tetrahydro-2H-pyran-4-yl)imidazo[5,1-f][1,2,4]triazin-4(3H)-one
(−)-2-((3,4-trans)-1-(imidazo[1,2-b]pyridazin-2-ylmethyl)-4-methylpyrrolidin-3-yl)-7-(tetrahydro-2H-pyran-4-yl)imidazo[5, 1-f][1,2,4]triazin-4(3H)-one
(−)-2-((3,4-trans)-1-(imidazo[1,2-b]pyridazin-6-ylmethyl)-4-methylpyrrolidin-3-yl)-7-(tetrahydro-2H-pyran-4-yl)imidazo[5, 1-f][1,2,4]triazin-4(3H)-one
(−)-2-((3,4-trans)-4-methyl-1-(pyrimidin-2-ylmethyl)pyrrolidin-3-yl)-7-(tetrahydro-2H-pyran-4-yl)imidazo[5,1-f][1,2,4]triazin-4(3H)-one
(+)-2-((3,4-trans)-4-methyl-1-(pyrimidin-2-ylmethyl)pyrrolidin-3-yl)-7-(tetrahydro-2H-pyran-4-yl)imidazo[5, 1-f][1,2,4]triazin-4(3H)-one
(−)-2-((3,4-trans)-4-methyl-1-(pyrazin-2-ylmethyl)pyrrolidin-3-yl)-7-(tetrahydro-2H-pyran-4-yl)imidazo[5, 1-f][1,2,4]triazin-4(3H)-one (+)-2-((3,4-trans)-4-methyl-1-(pyrazin-2-ylmethyl)pyrrolidin-3-yl)-7-(tetrahydro-2H-pyran-4-yl)imidazo[5, 1-f] [1,2,4]triazin-4(3H)-one
(+)-7-((1 s,3R)-adamantan-1-yl)-2-((3,4-trans)-4-methyl-1-(pyrimidin-2-ylmethyl)pyrrolidin-3-yl)imidazo[5,1-f][1,2,4]triazin-4(3H)-one
2-((3,4-trans)-1-benzyl-4-methylpyrrolidin-3-yl)-7-(tetrahydro-2H-pyran-3-yl)imidazo[5,1-f][1,2,4]triazin-4(3H)-one fraction 1
2-((3,4-trans)-1-benzyl-4-methylpyrrolidin-3-yl)-7-(tetrahydro-2H-pyran-3-yl)imidazo[5,1-fJ[1,2,4]triazin-4(3H)-one fraction 2
2-((3,4-trans)-1-benzyl-4-methylpyrrolidin-3-yl)-7-(tetrahydro-2H-pyran-3-yl)imidazo[5,1-fJ[1,2,4]triazin-4(3H)-one fraction 3
(+)-2-((3,4-trans)-1-((6-methoxypyridin-2-yl)methyl)-4-methylpyrrolidin-3-yl)-7-(tetrahydro-2H-pyran-4-yl)imidazo[5, 1-tj [1,2,4]triazin-4(3H)-one
(−)-2-((3,4-trans)-4-methyl-1-(pyrimidin-2-ylmethyl)pyrrolidin-3-yl)-7-(o-tolyl)imidazo[5, 1-fj [1,2,4]triazin-4(3H)-one
(−)-7-(4-fluoro-2-methylphenyl)-2-((3,4-trans)-4-methyl-1-(pyrimidin-2-ylmethyl)pyrrolidin-3-yl)imidazo[5, 1-fj [1,2,4]triazin-4(3H)-one
(+)-7-(4,4-difluorocyclohexyl)-2-((3,4-trans)-4-methyl-1-(pyrimidin-2-ylmethyl)pyrrolidin-3-yl)imidazo[5, 1-fj [1,2,4]triazin-4(3H)-one
(−)-2-((3,4-trans)-4-methyl-1-((R)-1-phenylethyl)pyrrolidin-3-yl)-7-(tetrahydro-2H-pyran-4-yl)imidazo[5, 1-fj [1,2,4]triazin-4(3H)-one (+)-2-((3,4-trans)-4-methyl-1-((S)-1-phenylethyl)pyrrolidin-3-yl)-7-(tetrahydro-2H-pyran-4-yl)imidazo[5, 1-f] [1, 2,4]triazin-4(3H)-one
(−)-7-(5-chloro-2-methylphenyl)-2-((3,4-trans)-4-methylpyrrolidin-3-yl)imidazo[5,1-f][1,2,4]triazin-4(3H)-one
(+)-2-((3,4-trans)-1-benzyl-4-methylpyrrolidin-3-yl)-7-(furan-3-yl)imidazo[5,1-f][1,2,4]triazin-4(3H)-one
(−)-2-((3,4-trans)-1-benzyl-4-methylpyrrolidin-3-yl)-7-(furan-3-yl)imidazo[5,1-f][1,2,4]triazin-4(3H)-one
(−)-7-(1-acetylpiperidin-4-yl)-2-((3,4-trans)-1-benzyl-4-methylpyrrolidin-3-yl)imidazo[5, 1-t][1,2,4]triazin-4(3H)-one
(+)-7-(1-acetylpiperidin-4-yl)-2-((3,4-trans)-1-benzyl-4-methylpyrrolidin-3-yl)imidazo[5, 1-f][1,2,4]triazin-4(3H)-one
(−)-2-((3,4-trans)-1-benzyl-4-methylpyrrolidin-3-yl)-7-(2-hydroxypropan-2-yl)imidazo[5, 1-f][1,2,4]triazin-4(3H)-one
(+)-2-((3,4-trans)-1-benzyl-4-methylpyrrolidin-3-yl)-7-(2-hydroxypropan-2-yl)imidazo[5, 1-f][1,2,4]triazin-4(3H)-one
(+)-2-((3,4-trans)-1-benzyl-4-methylpyrrolidin-3-yl)-7-(1-(ethylsulfonyl)piperidin-4-yl)imidazo[5, 1-f] [1,2,4]triazin-4(3H)-one
(−)-2-((3,4-trans)-1-benzyl-4-methylpyrrolidin-3-yl)-7-(1-(ethylsulfonyl)piperidin-4-yl)imidazo[5, 1-f][1,2,4]triazin-4(3H)-one (−)-7-(1-acetylpiperidin-4-yl)-2-((3,4-trans)-4-methyl-1-(pyrimidin-2-ylmethyl)pyrrolidin-3-yl)imidazo[5, 1-fj [1, 2,4]triazin-4(3H)-one
(+)-7-(1-acetylpiperidin-4-yl)-2-((3,4-trans)-4-methyl-1-(pyrimidin-2-ylmethyl)pyrrolidin-3-yl)imidazo[5, 1-fj [1,2,4]triazin-4(3H)-one
(+)-7-(1-(ethylsulfonyl)piperidin-4-yl)-2-((3,4-trans)-4-methyl-1-(pyrimidin-2-ylmethyl)pyrrolidin-3-yl)imidazo[5, 1-fj [1,2,4]triazin-4(3H)-one
(−)-7-(1-(ethylsulfonyl)piperidin-4-yl)-2-((3,4-trans)-4-methyl-1-(pyrimidin-2-ylmethyl)pyrrolidin-3-yl)imidazo[5, 1-fj [1,2,4]triazin-4(3H)-one
or a pharmaceutically acceptable salt thereof.

In some cases, PDE9 inhibitor is a compound having a structure of

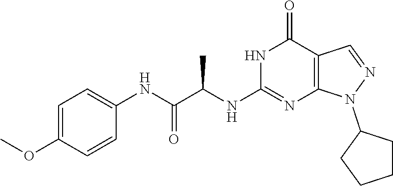 [SMILES: COc1ccc(NC(=O)[C@@H](C)Nc2nc3c(cnn3C3CCCC3)c(=O)[nH]2)cc1]

or a salt thereof.

In some cases, as disclosed in WO2020123271, PDE9 inhibitor is represented by a compound of formula (AA-III),

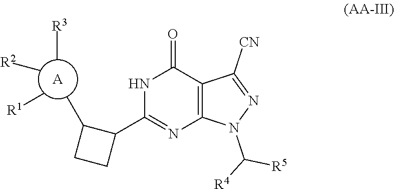

wherein:
A is a pyrazinyl, pyrazolyl, pyridyl, pyridyl-N-oxide, 5-pyrimidinyl, pyridazinyl, pyrrolopyrazolyl, dihydropyrrolopyrazolyl, morpholinyl, oxomorpholinyl, or oxadiazolyl ring;
R¹, R² and R³ as are present are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃,
   (3) —CF₃,
   (4) —CH₂OH,
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} as are present are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl, and
   (7) —CN; or a pharmaceutically acceptable salt thereof.

In some embodiments, PDE9 inhibitor is selected from the group consisting of
6-(2-(5-methyl-1,3,4-oxadiazol-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-methyl-1,3,4-oxadiazol-2-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-((1S,2R)-2-(5-methyl-1,3,4-oxadiazol-2-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-((1R,2S)-2-(5-methyl-1,3,4-oxadiazol-2-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile; 6-((1 S,2S)-2-(5-methyl-1,3,4-oxadiazol-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-methyl-1,3,4-oxadiazol-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1 S,2R)-2-(5-methyl-1,3,4-oxadiazol-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1R,2S)-2-(5-methyl-1,3,4-oxadiazol-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1 S,2S)-2-(5-methyl-1,3,4-oxadiazol-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
4-oxo-6-(2-(pyridin-2-yl)cyclobutyl)-1-(1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(pyridin-2-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(pyridin-2-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(pyridin-2-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(pyridin-2-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
2-(2-(3-cyano-4-oxo-1-(1-(6-(trifluoromethyl)pyridin-3-yl)ethyl)-4,5-dihydro-1H-pyrazolo[3,4-d]pyrimidin-6-yl)cyclobutyl)pyridine 1-oxide;
2-((1R,2R)-2-(3-cyano-4-oxo-1-((S)-1-(6-(trifluoromethyl)pyridin-3-yl)ethyl)-4, 5-dihydro-1H-pyrazolo[3,4-d]pyrimidin-6-yl)cyclobutyl)pyridine 1-oxide;
2-((1 S,2S)-2-(3-cyano-4-oxo-1-((S)-1-(6-(trifluoromethyl)pyridin-3-yl)ethyl)-4,5-dihydro-1H-pyrazolo[3,4-d]pyrimidin-6-yl)cyclobutyl)pyridine 1-oxide;
2-((1R,2R)-2-(3-cyano-4-oxo-1-((R)-1-(6-(trifluoromethyl)pyridin-3-yl)ethyl)-4,5-dihydro-1H-pyrazolo[3,4-d]pyrimidin-6-yl)cyclobutyl)pyridine 1-oxide;
2-((1 S,2S)-2-(3-cyano-4-oxo-1-((R)-1-(6-(trifluoromethyl)pyridin-3-yl)ethyl)-4,5-dihydro-1H-pyrazolo[3,4-d]pyrimidin-6-yl)cyclobutyl)pyridine 1-oxide; 6-(2-(5,6-dihydro-4H-pyrrolo[1,2-b]pyrazol-2-yl)cyclobutyl)-4-oxo-1-(1-(6-(trifluoromethyl)-pyridin-3-yl)ethyl)-4,5-dihydro-1H-pyrazolo[3,4-d]pyrimidine-3-carbonitrile;
6-((1R,2R)-2-(5,6-dihydro-4H-pyrrolo[1,2-b]pyrazol-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(5,6-dihydro-4H-pyrrolo[1,2-b]pyrazol-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5,6-dihydro-4H-pyrrolo[1,2-b]pyrazol-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1 S,2S)-2-(5,6-dihydro-4H-pyrrolo[1,2-b]pyrazol-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-fluoropyridin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-fluoropyridin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(5-fluoropyridin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-fluoropyridin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1 S,2S)-2-(5-fluoropyridin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(3-fluoropyridin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(3-fluoropyridin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(3-fluoropyridin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(3-fluoropyridin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1 S,2S)-2-(3-fluoropyridin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 6-(2-(1-methyl-1H-pyrazol-3-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(1-methyl-1H-pyrazol-3-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1 S,2S)-2-(1-methyl-1H-pyrazol-3-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(1-methyl-1H-pyrazol-3-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-((1 S,2S)-2-(1-methyl-1H-pyrazol-3-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-(2-(1-methyl-1H-pyrazol-5-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(1-methyl-1H-pyrazol-5-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1 S,2S)-2-(1-methyl-1H-pyrazol-5-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(1-methyl-1H-pyrazol-5-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-((1 S,2S)-2-(1-methyl-1H-pyrazol-5-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
4-oxo-6-(2-(pyrimidin-5-yl)cyclobutyl)-1-(1-(6-(trifluoromethyl)pyridin-3-yl)ethyl)-4, 5-dihydro-1H-pyrazolo[3,4-d]pyrimidine-3-carbonitrile;
4-oxo-6-((1R,2R)-2-(pyrimidin-5-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(pyrimidin-5-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(pyrimidin-5-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(pyrimidin-5-yl)cyclobutyl)-1-((R)-1-(6-(trifluoromethyl)pyridin-3-yl)ethyl)-
4,5-dihydro-1H-pyrazolo[3,4-d]pyrimidine-3-carbonitrile; 6-(2-(4-cyano-1H-pyrazol-1-yl)cyclobutyl)-4-oxo-1-(1-(6-(trifluoromethyl)pyridin-3-yl)ethyl)-4, 5-dihydro-1H-pyrazolo[3,4-d]pyrimidine-3-carbonitrile;
6-((1R,2R)-2-(4-cyano-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(4-cyano-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(4-cyano-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
6-((1 S,2S)-2-(4-cyano-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
6-(2-(3-cyano-1H-pyrazol-1-yl)cyclobutyl)-4-oxo-1-(1-(6-(trifluoromethyl)pyridin-3-yl)ethyl)-4, 5-dihydro-1H-pyrazolo[3,4-d]pyrimidine-3-carbonitrile;
6-((1R,2R)-2-(3-cyano-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(3-cyano-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(3-cyano-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
6-((1 S,2S)-2-(3-cyano-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
4-oxo-6-(2-(3-oxomorpholino)cyclobutyl)-1-(1-(6-(trifluoromethyl)pyri din-3-yl)ethyl)-4, 5-dihydro-1H-pyrazolo[3,4-d]pyrimidine-3-carbonitrile;
4-oxo-6-((1R,2R)-2-(3-oxomorpholino)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(3-oxomorpholino)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(3-oxomorpholino)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-((1 S,2S)-2-(3-oxomorpholino)cyclobutyl)-1-((R)-1-(6-(trifluoromethyl)pyridin-3-yl)ethyl)-4,5-dihydro-1H-pyrazolo[3,4-d]pyrimidine-3-carbonitrile; 4-oxo-6-(2-(3-(trifluoromethyl)-1H-pyrazol-1-yl)cyclobutyl)-1-(1-(6-(trifluoromethyl)pyri din-3-yl)ethyl)-4,5-dihydro-1H-pyrazolo[3,4-d]pyrimidine-3-carbonitrile;
4-oxo-6-((1R,2R)-2-(3-(trifluoromethyl)-1H-pyrazol-1-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1 S,2 S)-2-(3-(trifluorom ethyl)-1H-pyrazol-1-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(3-(trifluoromethyl)-1H-pyrazol-1-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-((1 S,2 S)-2-(3-(trifluorom ethyl)-1H-pyrazol-1-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
6-(2-(4-fluoro-1H-pyrazol-1-yl)cyclobutyl)-4-oxo-1-(1-(6-(trifluoromethyl)pyri din-3-yl)ethyl)-4,5-dihydro-1H-pyrazolo[3,4-d]pyrimidine-3-carbonitrile;
6-((1R,2R)-2-(4-fluoro-1H-pyrazol-1-yl)cyclobutyl)-4-oxo-1-((S)-1-(6-(trifluoromethyl)pyri din-3-yl)ethyl)-4,5-dihydro-1H-pyrazolo[3,4-d]pyrimidine-3-carbonitrile;
6-((1 S,2S)-2-(4-fluoro-1H-pyrazol-1-yl)cyclobutyl)-4-oxo-1-((S)-1-(6-(trifluoromethyl)pyri din-3-yl)ethyl)-4,5-dihydro-1H-pyrazolo[3,4-d]pyrimidine-3-carbonitrile;
6-((1R,2R)-2-(4-fluoro-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
6-((1 S,2S)-2-(4-fluoro-1H-pyrazol-1-yl)cyclobutyl)-4-oxo-1-((R)-1-(6-(trifluoromethyl)pyri din-3-yl)ethyl)-4,5-dihydro-1H-pyrazolo[3,4-d]pyrimidine-3-carbonitrile;
6-(2-(1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(1H-pyrazol-1-yl)cyclobutyl)-4-oxo-1-((R)-1-(6-(trifluorom ethyl)pyri din-3-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((R)-1-(6-(trifluorom ethyl)pyri din-3-yl)ethyl)-4,5-dihydro-1H-pyrazolo[3,4-d]pyrimidine-3-carbonitrile; 6-(2-(4-cyclopropyl-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(4-cyclopropyl-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1 S,2S)-2-(4-cyclopropyl-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1R,2R)-2-(4-cyclopropyl-1H-pyrazol-1-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 6-((1 S,2S)-2-(4-cyclopropyl-1H-pyrazol-1-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 6-(2-(4-cyano-1H-pyrazol-1-yl)cyclobutyl)-4-oxo-1-(1-(tetrahydro-2H-pyran-4-yl)ethyl)-4,5-dihydro-1H-pyrazolo[3,4-d]pyrimidine-3-carbonitrile;
6-((1R,2R)-2-(1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1R,2R)-2-(1H-pyrazol-1-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-(2-(4-(difluoromethyl)-1H-pyrazol-1-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R, 2R)-2-(4-(difluorom ethyl)-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1 S, 2S)-2-(4-(difluorom ethyl)-1H-pyrazol-1-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1R, 2R)-2-(4-(difluorom ethyl)-1H-pyrazol-1-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 6-((1 S, 2S)-2-(4-(difluorom ethyl)-1H-pyrazol-1-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 6-(2-(1H-pyrazol-1-yl)cyclobutyl)-4-oxo-1-(1-(tetrahy dro-2H-pyran-4-yl)ethyl)-4, 5-dihydro-1H-pyrazolo[3,4-d]pyrimidine-3-carbonitrile;
6-((1R,2R)-2-(1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1R,2R)-2-(1H-pyrazol-1-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-((1 S,2S)-2-(1H-pyrazol-1-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-(2-(4-cyano-1H-pyrazol-1-yl)cyclobutyl)-4-oxo-1-(1-(tetrahy dro-2H-pyran-4-yl)ethyl)-4, 5-dihydro-1H-pyrazolo[3,4-d]pyrimidine-3-carbonitrile;
6-((1R,2R)-2-(4-cyano-1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1 S,2S)-2-(4-cyano-1H-pyrazol-1-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1R,2R)-2-(4-cyano-1H-pyrazol-1-yl)cyclobutyl)-4-oxo-1-((R)-1-(tetrahy dro-2H-pyran-4-yl)ethyl)-4,5-dihydro-1H-pyrazolo[3,4-d]pyrimidine-3-carbonitrile; and
6-((1 S,2S)-2-(4-cyano-1H-pyrazol-1-yl)cyclobutyl)-4-oxo-1-((R)-1-(tetrahy dro-2H-pyran-4-yl)ethyl)-4,5-dihydro-1H-pyrazolo[3,4-d]pyrimidine-3-carbonitrile;
or a pharmaceutically acceptable salt thereof.

In some cases, as disclosed in WO2020123272, PDE9 inhibitor is represented by a compound of formula (AA-IV),

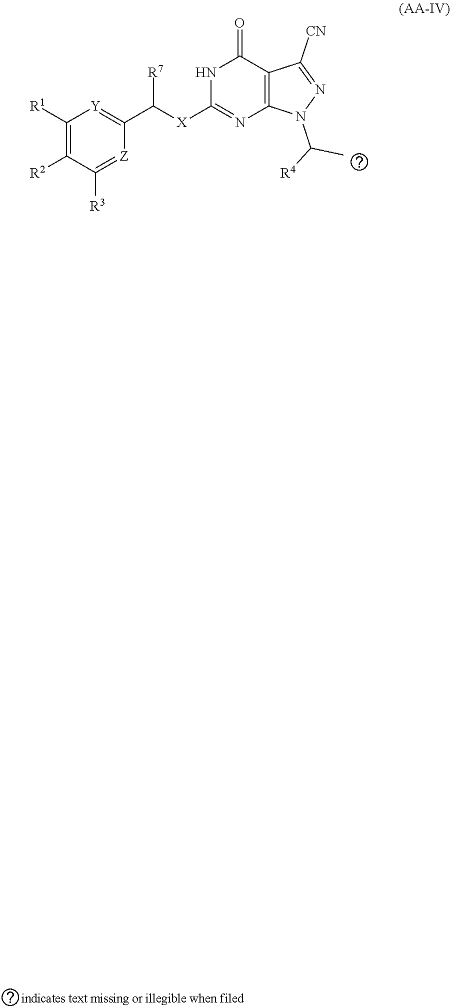

wherein:
X is —N(R⁶)— or —C(R⁸R⁹);
Y is —N═ or —CH═;
Z is —N═ or —CH═;
R¹, R² and R³ are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃;
   (3) —CF₃S
   (4) —CH₂OH,
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) azetidine, morpholine, piperidine, or pyrrolidine, and
   (8) —CN;
R⁶ is selected from:
   (1) hydrogen, and
   (2) C₁₋₆alkyl;
R⁷ is selected from:
   (1) hydrogen,
   (2) C₁₋₆alkyl, which is unsubstituted or substituted with substituents selected from: hydroxy and fluoro,
   (3) C₂₋₆alkenyl,
   (4) C₃₋₆cycloalkyl, which is unsubstituted or substituted with substituents selected from: hydroxy and fluoro, and
   (5) azetidine, morpholine, piperidine, or pyrrolidine;
or R⁶ and R⁷ and the —CH—N— to which they are attached are joined to form an azetidine or a pyrrolidine ring;
R⁸ and R⁹ are independently selected from:
   (1) hydrogen, and
   (2) C₁₋₆alkyl; or a pharmaceutically acceptable salt thereof.

In some embodiments, PDE9 inhibitor is selected from the group consisting of:

1-(1-(6-cyclopropylpyridin-3-yl)ethyl)-6-(methyl(1-(pyrimidin-2-yl)propyl)amino)-4-oxo-4,5-dihydro-1H-pyrazolo[3,4-d]pyrimidine-3-carbonitrile;
6-[methyl(1-pyrimidin-2-ylethyl)amino]-4-oxo-1-[1-[6-(trifluoromethyl)-3-pyridyl]ethyl]-5H-pyrazolo[3,4-d]pyrimidine-3-carbonitrile;
6-[methyl(1-pyrimidin-2-ylpropyl)amino]-4-oxo-1-[1-[6-(trifluoromethyl)-3-pyridyl]ethyl]-5H-pyrazolo[3,4-d]pyrimidine-3-carbonitrile;
6-[methyl(1-pyrimidin-2-ylpropyl)amino]-4-oxo-1-[1-[4-(trifluoromethyl)phenyl]ethyl]-5H-pyrazolo[3,4-d]pyrimidine-3-carbonitrile;
6-[1-[5-(difluoromethyl)pyrimidin-2-yl]propyl-methyl-amino]-4-oxo-1-[1-[6-(trifluoromethyl)-3-pyridyl]ethyl]-5H-pyrazolo[3,4-d]pyrimidine-3-carbonitrile;
6-[methyl-(2-methyl-1-pyrimidin-2-yl-propyl)amino]-4-oxo-1-[1-[6-(trifluoromethyl)-3-pyridyl]ethyl]-5H-pyrazolo[3,4-d]pyrimidine-3-carbonitrile;
6-[[cyclopropyl(pyrimidin-2-yl)methyl]-methyl-amino]-4-oxo-1-[1-[6-(trifluoromethyl)-3-pyridyl]ethyl]-5H-pyrazolo[3,4-d]pyrimidine-3-carbonitrile;
6-[[cyclobutyl(pyrimidin-2-yl)methyl]-methyl-amino]-4-oxo-1-[1-[6-(trifluoromethyl)-3-pyridyl]ethyl]-5H-pyrazolo[3,4-d]pyrimidine-3-carbonitrile;
6-[1-(5-fluoro-2-pyridyl)propyl-methyl-amino]-4-oxo-1-[1-[6-(trifluoromethyl)-3-pyridyl]ethyl]-5H-pyrazolo[3,4-d]pyrimidine-3-carbonitrile;
6-[1-(5-fluoropyrimidin-2-yl)propyl-methyl-amino]-4-oxo-1-(1-tetrahydropyran-4-ylethyl)-5H-pyrazolo[3,4-d]pyrimidine-3-carbonitrile;
6-[methyl(1-pyrimidin-2-ylpent-4-enyl)amino]-4-oxo-1-[1-[6-(trifluoromethyl)-3-pyridyl]ethyl]-5H-pyrazolo[3,4-d]pyrimidine-3-carbonitrile;
4-oxo-6-(2-pyrimidin-2-ylazetidin-1-yl)-1-[1-[6-(trifluoromethyl)-3-pyridyl]ethyl]-5H-pyrazolo[3,4-d]pyrimidine-3-carbonitrile;
6-[2-(4-fluorophenyl)azetidin-1-yl]-4-oxo-1-[1-[6-(trifluoromethyl)-3-pyridyl]ethyl]-5H-pyrazolo[3,4-d]pyrimidine-3-carbonitrile;
4-oxo-6-(2-pyrimidin-2-ylpyrrolidin-1-yl)-1-[1-[6-(trifluoromethyl)-3-pyridyl]ethyl]-5H-pyrazolo[3,4-d]pyrimidine-3-carbonitrile;
6-[2-(4-fluorophenyl)azetidin-1-yl]-4-oxo-1-[1-[6-(trifluoromethyl)-3-pyridyl]ethyl]-5H-pyrazolo[3,4-d]pyrimidine-3-carbonitrile; 6-[methyl-[1-[5-(trifluoromethoxy)pyrimidin-2-yl]propyl]amino]-4-oxo-1-[1-[6-(trifluoromethyl)-3-pyridyl]ethyl]-5H-pyrazolo[3,4-d]pyrimidine-3-carbonitrile;
1-[1-(6-cyclopropyl-3-pyridyl)ethyl]-6-[1-(5-fluoropyrimidin-2-yl)propyl-methyl-amino]-4-oxo-5H-pyrazolo[3,4-d]pyrimidine-3-carbonitrile;
1-[1-(4-cyclopropylphenyl)ethyl]-6-[1-(5-fluoropyrimidin-2-yl)propyl-methyl-amino]-4-oxo-5H-pyrazolo[3,4-d]pyrimidine-3-carbonitrile;
4-oxo-6-((1-(pyrimidin-2-yl)propyl)amino)-1-(1-(6-(trifluoromethyl)pyridin-3-yl)ethyl)-4, 5-dihydro-1H-pyrazolo[3,4-d]pyrimidine-3-carbonitrile;
6-((1-(5-fluoropyrimidin-2-yl)propyl)(methyl)amino)-4-oxo-1-(1-(6-(trifluoromethyl)pyri din-3-yl)ethyl)-4,5-dihydro-1H-pyrazolo[3,4-d]pyrimidine-3-carbonitrile;
4-oxo-6-(2-(pyrimidin-2-yl)ethyl)-1-(1-(6-(trifluoromethyl)pyridin-3-yl)ethyl)-4, 5-dihydro-1H-pyrazolo[3,4-d]pyrimidine-3-carbonitrile;
6-(2-(5-cyclopropylpyrimidin-2-yl)ethyl)-4-oxo-1-(1-(6-(trifluoromethyl)pyridin-3-yl)ethyl)-4,5-dihydro-1H-pyrazolo[3,4-d]pyrimidine-3-carbonitrile;
4-oxo-6-(2-(pyrimidin-2-yl)propyl)-1-(1-(6-(trifluoromethyl)pyridin-3-yl)ethyl)-4,5-dihydro-1H-pyrazolo[3,4-d]pyrimidine-3-carbonitrile; and
4-oxo-6-(2-(pyrimidin-2-yl)butyl)-1-(1-(6-(trifluoromethyl)pyridin-3-yl)ethyl)-4, 5-dihydro-1H-pyrazolo[3,4-d]pyrimidine-3-carbonitrile;
or a pharmaceutically acceptable salt thereof.

In some cases, as disclosed in WO2018226771, PDE9 inhibitor is represented by a compound of formula (AA-V),

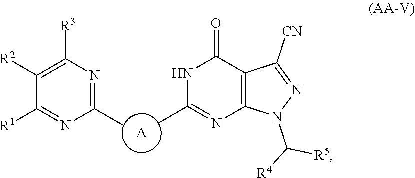

wherein
A is a cyclobutyl ring, which is unsub substituted or or substituted with substituents selected from: fluoro and methyl;
R¹, R² and R³ are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃;
   (3) —CF₃,
   (4) —CH₂OH.
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl. pyridazinyl, thiazolyl, cyclohexyl or tetrahydropyranyl ring is substituted with R^{1a}, R^{1b} and R^{1c}, wherein R^{1a} R^{1b} and R^{1c} are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl, and
   (7) —CN; pharmaceutically acceptable salt thereof.

In some embodiments, PDE9 inhibitor is selected from the group consisting of;
1-(1-(4-fluorophenyl)ethyl)-4-oxo-6-(2-(pyrimidin-2-yl)cyclobutyl)-4,5-dihydro-1H-pyrazolo[3,4-d]pyrimidine-3-carbonitrile;
1-((S)-1-(4-fluorophenyl)ethyl)-4-oxo-6 (1R,2R)-2-(pyrimidin-2-yl)cyclobutyl)-4,5-dihydro-1H-pyrazolo[3,4-d]pyrimidine-3-carbonitrile;
1-((S)-1-(4-fluorophenyl ethyl)-4-oxo-6-((1S,2S)-2-(pyrimidin-2-yl)cyclobutyl)-4,5-dihydro-11H-pyrazolo[3,4-d]pyrimidine-3-carbonitrile;
1-((R)-1-(4-fluorophenyl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(4-fluorophenyl)ethyl)-4-oxo-6-((1S,2S)-2-(pyrimidin-2-yl)cyclobutyl)-4,5-dihydro-1H-pyrazolo[3,4-d]pyrimidine-3-carbonitrile,
4-oxo-6-(2-(pyrimidin-2-yl)cyclobutyl)-1-(1-(6-(trifluormethyl)pyridin-3-yl)ethyl)-4,5-dihydro 1H-pyrazolo[3,4-d]pyrimidine-3-carbonitrile;
4-oxo-6-((1R,2R)-2-(pyrimidin-2-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(6-(trifluoromethyl)pyridin-3-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((R)-1-(6-(trifluoromethyl)pyridin-3-yl)ethyl)-4,5-dihydro-1H-pyrazolo[3,4-d]pyrimidine-3-carbonitrile;
4-oxo-6-(2-(pyrimidin-2-yl)cyclobutyl)-1-((6-(trifluoromethyl)pyridin-3-yl)methyl)-4,5-dihydro 1H-pyrazolo[3,4-d]pyrimidine-3-carbonitrile;
4-oxo-6-((1R,2R)-2-(pyrimidin-2-yl)cyclobutyl)-1-((6-(trifluoromethyl)pyridin-3-yl)methyl)-4,5-dihydro-1H-pyrazolo[3,4-d]pyrimidine-3-carbonitrile;
4-oxo-6-((1S,2S)-2-(pyrimidin-2-yl)cyclobutyl)-1-((6 trifluoromethyl)pyridin-3-yl)methyl)-4,5-dihydro-1H-pyrazolo[3,4-d]pyrimidine-3-carbonitrile;
1-[1-(4-cyanophenyl)ethyl]-4-oxo-6-(2-pyrimidin-2-yl)cyclobutyl)-4,5-dihydro-1H-pyrazolo[3,4-d]pyrimidine-3-carbonitrile;
1-((S)-1-(4-cyanophenyl)ethyl)-4-oxo-6-((1R,2R)-2-(pyrimidin-2-yl)cyclobutyl)-4,5-dihydro-1H-pyrazolo[3,4-d]pyrimidine-3-carbonitrile; 1-((S)-1-(4-cyanophenyl)ethyl)-4-oxo-6-((1S,2S)-2-(pyrimidin-2-yl)cyclobutyl)-4,5-dihydro-1H pyrazolo[3,4-d]pyrimidine-3-carbonitrile;
1-((R)-1-(4-cyanophenyl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(4-cyanophenyl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-(1-(3,4-difluorophenyl)ethyl)-4-oxo-6-(2-(pyrimidin-2-yl)cyclobutyl)-4,5-dihydro-1H-pyrazolo[3,4-d]pyrimidine-3-carbonitrile;
1-((S)-1-(3,4-difluorophenyl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((S)-1-(3,4-difluorophenyl)ethyl)-4-oxo-6-((1S,2S)-2-(pyrimidin-2-yl)cyclobutyl)-4,5-dihydro 1H-pyrazolo[3,4-d]pyrimidine-3-carbonitride;
1-((R)-1-(3,4-difluorophenyl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(3,4-difluorophenyl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
4-oxo-6-[2-pyrimidin-2-ylcyclobutyl]-1-{1-[4-(trifluoromethyl)phenyl]ethyl}-4,5-dihydro-1H-pyrazolo[3,4-d]pyrimidine-3-carbonitrile;
4-oxo-6-((1R,2R)-2-(pyrimidin-2-yl)cyclobutyl)-1-((S)-1-(4-(trifluoromethyl)phenyl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(4-(trifluoromethyl)phenyl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(4-(trifluoromethyl)phenyl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((R)-1-(4-(trifluoromethyl)phenyl)ethyl)-4,5-dihydro-1H-pyrazolo[3,4-d]pyrimidine-3-carbonitrile;
4-oxo-6-[2-pyrimidin-2-cyclobutyl]-1-{1-[3-(trifluoromethyl)phenyl]ethyl}-4,5-dihydro-1H-pyrazolo[3,4-d]pyrimidine-3-carbonitrile;
4-oxo-6-((1R,2-R)-2-(pyrimidin-2-yl)cyclobutyl)-1-((S)-1-(3-(trifluoromethyl)phenyl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(3-(trifluoromethyl)phenyl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(3-(trifluoromethyl)phenyl)ethyl)-4,5-dihydro-Ili-pyrazolo[3,4-d]pyrimidine-3-carbonitrile;
4-oxo-6-((1S,2S)-2-(pyrimidin-2-yl)cyclobutyl)-1-((R)-1-(3-(trifluoromethyl)phenyl)ethyl)-4,5-dihydro-1H-pyrazolo[3,4-d]pyrimidine-3-carbonitrile;
4-oxo-6-(2-(pyrimidin-2-yl)cyclobutyl)-1-(1-(5-(trifluoromethyl)pyrazin-2-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((S)-1-(5-(trifluoromethyl)pyrazin-2-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(5-(trifluoromethyl)pyrazin-2-yl)ethyl)-4,5-dihydro-1H-pyrazolo[3,4-d]pyrimidine-3-carbonitrile;
4-oxo-64(1R,2R)-2-(pyrimidin-2-yl)cyclobutyl)-1-((R)-1-(5-(trifluoromethyl)pyrazin-2-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((R)-1-(5-(trifluoromethyl)pyrazin-2-yl)ethyl)-4,5-dihydro-1H-pyrazolo[3,4-d]pyrimidine-3-carbonitrile;
6-(2-(5-methylpyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-methylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 5-methylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
(6-((1R,2R)-2-(5-methylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2 5-methylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
4-oxo-6-(2-(pyrimidin-2-yl)cyclobutyl)-1-(1-(2-(trifluoromethyl)pyrimidin-5-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((S)-1-(2 trifluoromethyl)pyrimidin-5-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(2-(trifluoromethyl)pyrimidin-5-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(2 trifluoromethyl)pyrimidin-5-yl)ethyl)-4,5-dihydro-1H-pyrazolo[3,4-d]pyrimidine-3-carbonitrile; 4-oxo-6-((1S,2S)-2-(pyrimidin-2-yl)cyclobutyl)-1-((R)-1-(2-(trifluoromethyl)pyrimidin-5-yl)ethyl)-4,5-dihydro-1H-pyrazolo[3,4-d]pyrimidine-3-carbonitrile;
1-(1-(6-(difluoromethyl)pyridin-3-yl)ethyl)-4-o\hat{ }
1H-pyrazolo[3,4-d]pyrimidine-3-carbonitrile;
1-((S)-1-(6 difluoromethyl)pyridin-3-yl)ethyl)-4-oxo-6-((1R,2R)-2 pyrimidin-2-yl)cyclobutyl) 4,5-dihydro-1H-pyrazolo[3,4-d]pyrimidine-3-carbonitrile;
1-(S)-1-(6 difluoromethyl)pyridin-3-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((R)-1-(6-(difluoromethyl)pyridin-3-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(6-(difluoromethyl)pyridin-3-yl)ethyl)-4-oxo-6-((1S,2S)-2 pyrimidin-2-yl)cyclobutyl) 4,5-dihydro-1H-pyrazolo[3,4-d]pyrimidine-3-carbonitrile;
1-((S)-1-(6 difluoromethyl)pyridin-3-yl)ethyl)-4-oxo-6-((1R,2S)-2 pyrimidin-2-yl)cyclobutyl) 4,5-dihydro-1H-pyrazolo[3,4-d]pyrimidine-3-carbonitrile;
1-((S)-1-(6 difluoromethyl)pyridin-3-yl)ethyl)-4-oxo-6-((1S,2R)-2 pyrimidin-2-yl)cyclobutyl) 4,5-dihydro-1H-pyrazolo[3,4-d]pyrimidine-3-carbonitrile;
1-((R)-1-(6-(difluoromethyl)pyridin-3-yl)ethyl)-4-oxo-6-((1R,2S)-2 pyrimidin-2-yl)cyclobutyl) 4,5-dihydro-1H-pyrazolo[3,4-d]pyrimidine-3-carbonitrile;
1-((R)-1(6-(difluoromethyl)pyridin-3-yl)ethyl)-4-oxo-6-((1S,2R)-2 pyrimidin-2-yl)cyclobutyl) 4,5-dihydro-1H-pyrazolo[3,4-d]pyrimidine-3-carbonitrile;
4-oxo-6-(2-(pyrimidin-2-yl)cyclobutyl)-1-(1-(tetrahydro-2H-pyran-4-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((S)-1-(tetrahydro-2H-pyran-4-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(tetrahydro-2H-pyran-4-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(tetrahydro-2H-pyran-4-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((R)-1-(tetrahydro-2H-pyran-4-yl)ethyl)-4,5-dihydro-1H-pyrazolo[3,4-d]pyrimidine-3-carbonitrile;
1-(1-(6-cyclopropylpyridin-3-yl)ethyl)-4-oxo-6-(2-(pyrimidin-2-yl)cyclobutyl)-4,5-dihydro-1H-pyrazolo[3,4-d]pyrimidine-3-carbonitrile; 1-((S)-1-(6-cyclopropylpyridin-3-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((S)-1-(6-cyclopropylpyridin-3-yl)ethyl)-4-oxo-6-(1S,2S)-2-(pyrimidin-2-yl)cyclobutyl)-4,5-dihydro-1H-pyrazolo[3,4-d]pyrimidine-3-carbonitrile;
1-((R)-1-(6-cyclopropylpyridin-3-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(6-cyclopropylpyridin-3-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((S)-1-(6-cyclopropylpyridin-3-yl)ethyl)-4-oxo-6-((1R,2S)-2-(pyrimidin-2-yl)cyclobutyl)-4,5-dihydro-1H-pyrazolo[3,4-d]pyrimidine-3-carbonitrile;
1-((S)-1-(6-cyclopropylpyridin-3-yl)ethyl)-4-oxo-6-((1S,2R)-2-(pyrimidin-2-yl)cyclobutyl)-4,5-dihydro-1H-pyrazolo[3,4-d]pyrimidine-3-carbonitrile;
1-((R)-1-(6-cyclopropylpyridin-3-yl)ethyl)-4-oxo-6-((1R,2S)-2-(pyrimidin-2-yl)cyclobutyl)-4,5-dihydro-1H-pyrazolo[3,4-d]pyrimidine-3-carbonitrile;
1-((R)-1-(6-cyclopropylpyridin-3-yl)ethyl)-4-oxo-6-((1S,2R)-2-(pyrimidin-2-yl)cyclobutyl)-4,5-dihydro-1H-pyrazolol[3,4-d]pyrimidine-3-carbonitrile;
6-(2-(5-bromopyrimidin-2-yl)cyclobutyl)-4-oxo-1-(1-(6-trifluoromethyl)pyridin-3-yl)ethyl)-4,5-dihydro-1H-pyrazolo[3,4-d]pyrimidine-3-carbonitrile;
6-((1R,2R)-2-(5-bromopyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 5-bromopyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-bromopyrimidin-2-yl)cyclobutyl)-4-oxo-1-((R)-1-(6-(trifluoromethyl)pyridin-3 yl)ethyl)-4,5-dihydro-1H-pyrazolo[3,4-d]pyrimidine-3-carbonitrile;
6-((1S,2S)-2-(5-bromopyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-(difluoromethoxy)pyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-(difluoromethoxy)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-(difluoromethoxy)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1R,2R)-2-(5-(difluoromethoxy)pyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-(difluoromethoxy)pyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-ethylpyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-ethylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 5-ethylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-ethylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2 5-ethylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
1-(1-(5-fluoropyridin-3-yl)ethyl)-4-oxo-6-(2-(pyrimidin-2-yl)cyclobutyl)-4,5-dihydro-H-pyrazolo[3,4-d]pyrimidine-3-carbonitrile;
1-((S)-1-(5-fluoropyridin-3-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((S)-1-(5-fluoropyridin-3-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((R)-1-(5-fluoropyridin-3-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(5-fluoropyridin-3-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-(1-(1-methyl-1H-pyrazol-4-yl)ethyl)-4-oxo-6-(2-(pyrimidin-2-yl)cyclobutyl)-4,5-dihydro-1H-pyrazolo[3,4-d]pyrimidine-3-carbonitrile;
1-((S)-1-(1-methyl-1H-pyrazol-4-yl)ethyl)-4-oxo-6 (1R,2R)-2-(pyrimidin-2-yl)cyclobutyl)-4,5-dihydro-1H-pyrazolo[3,4-d]pyrimidine-3-carbonitrile;
1-((S)-1-(1-methyl-1H-pyrazol-4-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((R)-1-(1-methyl-1H-pyrazol-4-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile; 1-((R)-1-(1-methyl-1H-pyrazol-4-yl)ethyl)-4-oxo-6-((1 S,2S)-2-(pyrimidin-2-yl)cyclobutyl)-4,5-dihydro-1H-pyrazolo[3,4-d]pyrimidine-3-carbonitrile;
1-(1-(6-cyclopropylpyridin-3-yl)ethyl)-6-(2-(5-fluoropyrimidin-2-yl)cyclobutyl)-4-oxo-4,5-dihydro-1H-pyrazolo[3,4-d]pyrimidine-3-carbonitrile;
1-((S)-1-(6-cyclopropylpyridin-3-yl)ethyl)-6-((1R,2R)-2-(5-fluoropyrimidin-2-yl)cyclobulyl)-4-oxo-4,5-dihydro-1H-pyrazolo[3,4-d]pyrimidine-3-carbonitrile;
1-((S)-1-(6-cyclopropylpyridin-3-yl)ethyl)-6 (1S,2S)-2-(5-fluoropyrimidin-2-yl)cyclobutyl)-4-oxo-4,5-dihydro-1H-pyrazolo[3,4-d]pyrimidine-3-carbonitrile;
1-((R)-1-(6-cyclopropylpyridin-3-yl)ethyl)-6-((1R,2R)-2 5-fluoropyrimidin-2-yl)cyclobutyl)-4-oxo-4,5-dihydro-1H-pyrazolo[3,4-d]pyrimidine-3-carbonitrile;
1-((R)-1-(6-cyclopropylpyridin-3-yl)ethyl)-6-((1S,2S)-2-(5-fluoropyrimidin-2-yl)cyclobutyl)-4-oxo-4,5-dihydro-1H-pyrazolo[3,4-d]pyrimidine-3-carbonitrile;
4-oxo-6-(2-(pyrimidin-2-yl)cyclobutyl)-1-(1-(2-(trifluoromethyl)thiazol-5-yl)ethyl)-4,5-dihydro 1H-pyrazolo[3,4-d]pyrimidine-3-carbonitrile;
4-oxo-6-((1R,2R)-2-(pyrimidin-2-yl)cyclobutyl)-1-((S)-1-(2-(trifluoromethyl)thiazol-5-yl)ethyl) 4,5-dihydro-1H-pyrazolo[3,4-d]pyrimidine-3-carbonitrile;
4-oxo-6-((1S,2S)-2-(pyrimidin-2-yl)cyclobutyl)-1-((S)-1-(2-(trifluoromethyl)thiazol-5-yl)ethyl)-4,5-dihydro-1H-pyrazolo[3,4-d]pyrimidine-3-carbonitrile;
4-oxo-6-((1R,2R)-2-(pyrimidin-2-yl)cyclobutyl)-1-((R)-1-(2-(trifluoromethyl)thiazol-5-yl)ethyl)-4,5-dihydro-1H-pyrazolo[3,4-d]pyrimidine-3-carbonitrile;
4-oxo-6-((1S,2S)-2-(pyrimidin-2-yl)cyclobutyl)-1-((R)-1-(2-(trifluoromethyl)thiazol-5-yl)ethyl)-4,5-dihydro-1H-pyrazolo[3,4-d]pyrimidine-3-carbonitrile;
1-(1-(2-cyclopropylpyrimidin-5-yl)ethyl)-6-(2-(5-fluoropyrimidin-2-yl)cyclobutyl)-4-oxo-4,5-dihydro-1H-pyrazolo[3,4-d]pyrimidine-3-carbonitrile;
1-((S)-1-(2-cyclopropylpyrimidin-5-yl)ethyl)-6-((1R,2R)-2-(5-fluoropyrimidin-2-yl)cyclobutyl)-4-oxo-4,5-dihydro-1H-pyrazolo[3,4-d]pyrimidine-3-carbonitrile;
1-((S)-1-(2-cyclopropylpyrimidin-5-yl)ethyl)-6-(1S,2S)-2-(5-fluoropyrimidin-2-yl)cyclobutyl)-4-oxo-4,5-dihydro-1H-pyrazolo[3,4-d]pyrimidine-3-carbonitrile;
1-((R)-1-(2-cyclopropylpyrimidin-5-yl)ethyl)-6-((1R,2R)-2-(5-fluoropyrimidin-2-yl)cyclobutyl)-4-oxo-4,5-dihydro-1H-pyrazolo[3,4-d]pyrimidine-3-carbonitrile;
1-((R)-1-(2-cyclopropylpyrimidin-5-yl)ethyl)-6-((1S,2S)-2-(5-fluoropyrimidin-2-yl)cyclobutyl)-4-oxo-4,5-dihydro-1H-pyrazolo[3,4-d]pyrimidine-3-carbonitrile; 6-(2-(5-ethynylpyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-ethynylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 5-ethynylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-ethynylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2 5-ethynylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-fluoropyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-fluoropyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 5-fluoropyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-fluoropyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2 5-fluoropyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-fluoropyrimidin-2-yl)cyclobutyl)-4-oxo-1-(1-(tetrahydro-2H-pyran-4-yl)ethyl)-4,5-dihydro-H-pyrazolo[3,4-d]pyrimidine-3-carbonitrile;
6-((1R,2R)-2-(5-fluoropyrimidin-2-yl)cyclobutyl)-4-oxo-1-((S)-1-(tetrahydro-2H-py-ran-4-yl)ethyl)-4,5-dihydro-1H-pyrazolo[3,4-d]pyrimidine-3-carbonitrile;
6-((1S,2S)-2-(5-fluoropyrimidin-2-yl)cyclobutyl)-4-oxo-1-((S)-1-(tetrahydro-2H-pyran-4-yl)ethyl)-4,5-dihydro-1H-pyrazolo[3,4-d]pyrimidine-3-carbonitrile;
6-((1R,2R)-2-(5-fluoropyrimidin-2-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-((1S,2S)-2-(5-fluoropyrimidin-2-yl)cyclobutyl)-4-oxo-1-((R)-1-(tetrahydro-2H-pyran-4-yl)ethyl)-4,5-dihydro-1H-pyrazolo[3,4-d]pyrimidine-3-carbonitrile;
6-(1-methyl-2-(pyrimidin-2-yl)cyclobutyl)-4-oxo-1-(1-(6-trifluoromethyl)pyridin-3-yl)ethyl)-4,5-dihydro-1H-pyrazolo[3,4-d]pyrimidine-3-carbonitrile; 6-((1R,2R)-1-methyl-2-(pyrimidin-2-ylcyclobutyl)-4-oxo-1-((S)-1-(6 trifluoromethyl)pyridin-3-yl)ethyl)-4,5-dihydro-1H-pyrazolo[3,4-d]pyrimidine-3-carbonitrile;
6-((1S,2S)-1-methyl-2-(pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-1-methyl-2-(pyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-1-methyl-2-(pyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-cyanopyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-cyanopyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-cyanopyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-cyanopyrimidin-2-yl)cyclobutyl)-4-oxo-1-((R)-1-(6-(trifluoromethyl)pyridin-3 yl)ethyl)-4,5-dihydro-1H-pyrazolo[3,4-d]pyrimidine-3-carbonitrile;
6-((1S,2S)-2-(5-cyanopyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-(2-(5-cyclopropylpyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-cyclopropylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-cyclopropylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-cyclopropylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-cyclopropylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile; 1-(1-(2-cyclopropylpyrimidin-5-yl)ethyl)-4-oxo-6-(2-(pyrimidin-2-yl)cyclobutyl)-4,5-dihydro-1H-pyrazolo[3,4-d]pyrimidine-3-carbonitrile;
1-((S)-1-(2-cyclopropylpyrimidin-5-yl)ethyl)-4-oxo-6-((1R,2R)-2-(pyrimidin-2-yl)cyclobutyl)-4,5-dihydro-1H-pyrazolo[3,4-d]pyridine-3-carbonitrile; 1-((S)-1-(2-cyclopropylpyrimidin-5-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((R)-1-(2-cyclopropylpyrimidin-5-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((R)-1-(2-cyclopropylpyrimidin-5-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
6-(2-(5-methoxypyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-methoxypyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-methoxypyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-methoxypyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-methoxypyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
1-(1-(5-fluoropyridin-2-yl)ethyl)-4-oxo-6-(2-(pyrimidin-2-yl)cyclobutyl)-4,5-dihydro-H-pyrazolo[3,4-d]pyrimidine-3-carbonitrile;
1-((S)-1-(5-fluoropyridin-2-yl)ethyl)-4-oxo-6-((1R,2R)-2-(pyrimidin-2-yl)cyclobutyl)-4,5-dihydro-1H-pyrazolo[3,4-d]pyrimidine-3-carbonitrile;
1-((S)-1-(5-fluoropyridin-2-yl)ethyl)-4-oxo-6-((1S,2S)-2-(pyrimidin-2-yl)cyclobutyl)-4,5-dihydro-1H-pyrazolo[3,4-d]pyrimidine-3-carbonitrile;
1-((S)-1-(5-fluoropyridin-2-yl)ethyl)-4-oxo-6-((1R,2S)-2-(pyrimidin-2-yl)cyclobutyl)-4,5-dihydro-1H-pyrazolol[3,4-d]pyrimidine-3-carbonitrile;
1-((S)-1-(5-fluoropyridin-2-yl)ethyl)-4-oxo-6-((1S,2R)-2-(pyrimidin-2-yl)cyclobutyl)-4,5-dihydro-1H-pyrazolo[3,4-d]pyrimidine-3-carbonitrile;
6-(2-(5-(difluoromethyl)pyrimidin-2-ylcyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-(difluoromethyl)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-(difluoromethyl)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile; 6-((1R,2S)-2-(5-(difluoromethyl)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2R)-2-(5-(difluoromethyl)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-(2-(5-chloropyrimidin-2-yl)cyclobutyl)-4-oxo-1-(1-(6-(trifluoromethyl)pyridin-3-yl)ethyl)-4,5-dihydro-1H-pyrazolo[3,4-d]pyrimidine-3-carbonitrile;
6-((1R,2R)-2-(5-chloropyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-chloropyrimidin-2-yl)cyclobutyl)-4-oxo-1-(S)-1-(6-(trifluoromethyl)pyridin-3-yl)ethyl)-4,5-dihydro-1H-pyrazolo[3,4-d]pyrimidine-3-carbonitrile;
6-((1R,2R)-2-(5-chloropyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2-(5-chloropyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1S,2S)-2 4-methylpyrimidin-2-yl)-cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2-(4,6-dimethylpyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1S,2S)-2 4 difluoromethyl)pyrimidin-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1S,2S)-2-(4,6-di-tert-butylpyrimidin-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1S,2S)-2 4-cyclopropylpyrimidin-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
6-((1R,2R)-2-(4-(difluoromethyl)pyrimidin-2-yl)cyclobutyl)-4-oxo-1-((R)-1-(6-(trifluoromethyl)-pyridin-3-yl)ethyl)-4,5-dihydro-1H-pyrazolo[3,4-d]pyrimidine-3-carbonitrile;
6-((1R,2R)-2-(4,6-bis(difluoromethyl)pyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1R,2R)-2-(4-methylpyrimidin-2-yl)cyclobutyl)-4-oxo-1-((R)-1-(6-(trifluoromethyl)pyridin-3-yl)ethyl)-4,5-dihydro-1H-pyrazolo[3,4-d]pyrimidine-3-carbonitrile;
6-((1R,2R)-2-(4,6-di-tert-butylpyrimidin-2-yl)cyclobutyl)-4-oxo-1-((R)-1-(6 trifluoromethyl)-pyridin-3-yl)ethyl)-4,5-dihydro-1H-pyrazolo[3,4-d]pyrimidine-3-carbonitrile; 6-((1R,2R)-2-(4-methylpyrimidin-2-yl)cyclobutyl)-4-oxo-1-((S)-1-(6 trifluoromethyl)pyridin-3-yl)ethyl)-4,5-dihydro-1H-pyrazolo[3,4-d]pyrimidine-3-carbonitrile;
6-((1R,2R)-2-(4,6-dimethylpyrimidin-2-yl)cyclobutyl)-4-oxo-1-((S)-1-((6-(trifluoromethyl)pyridin-3-yl)ethyl)-4,5-dihydro-1H-pyrazolo[3,4-d]pyriniidine-3-carbonitrile; 6-(2-(4-(tert-butyl)pyrimidin-2-yl)cyclobutyl)-4-oxo-1-((S)-1-(6-(trifluoromethyl)pyridin-3-yl)ethyl)-4,5-dihydro-1H-pyrazolo[3,4-d]pyrimidine-3-carbonitrile;
6-((1R,2R)-2-(4,6-di-tert-bufylpyriniidin-2-yl)cyclobufyl)-4-oxo-1-((S)-1-(6 trifluorom pyridin-3-yl)ethyl)-4,5-dihydro-1H-pyrazolo[3,4-d]pyrimidine-3-carbonitrile;
6-((1R,2R)-2-(4-(difluoromethyl)pyrimidin-2-yl)cyclobutyl)-4-oxo-1-((S)-1-(6-(trifluoromethyl)-pyridin-3-yl)ethyl)-4,5-dihydro-1H-pyrazolo[3,4-d]pyriniidine-3-carbonitrile; and
6-((1R,2R)-2-(4-cyclopropylpyriniidin-2-yl)cyclobutyl)-4-oxo-1-((S)-1-(6-(trifluoromethyl)-pyridin-3-yl)ethyl)-4,5-dihydro-1H-pyrazolo[3,4-d]pyrimidine-3-carbonitrile;
or a pharmaceutically acceptable salt thereof.

In some cases, as disclosed in U.S. Ser. No. 11/028,092, PDE9 inhibitor is selected from the group consisting of:

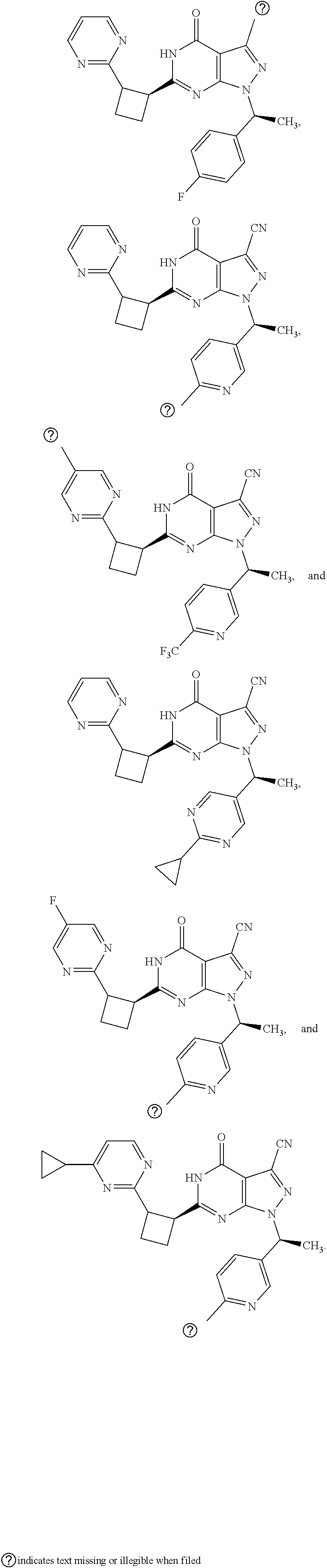

In some cases as disclosed in WO2017019723, PDE9 inhibitor is represented by a compound of formula (AA-VI),

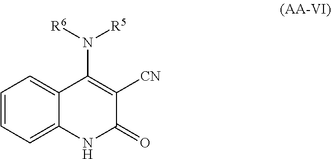

wherein
R⁵ is selected from the group consisting of:
   (1) hydrogen, and
   (2) —C₁₋₆alkyl;
R⁶ is —C₁₋₆alkyl, which is unsubstituted or substituted with oxetane;
or R⁵ and R⁶ are joined together with a —C₃₋₆alkyl through the nitrogen atom to which they are attached to form a azetidine, pyrrolidine, piperidine, or azepan ring, which is unsubstituted or substituted with pyrazole or one or two —C₁₋₆alkyl;
or a pharmaceutically acceptable salt thereof.

In some embodiments, PDE9 inhibitor is selected from the group consisting of:

In some embodiments, PDE9 inhibitor is selected from the group consisting of
(R)-4-(3-(1H-pyrazol-1-yl)pyrrolidin-1-yl)-2-oxo-1,2-dihydroquinoline-3-carbonitrile;
4-(azepan-1-yl)-2-oxo-1,2-dihydroquinoline-3-carbonitrile;
4-(azetidin-1-yl)-2-oxo-1,2-dihydroquinoline-3-carbonitrile;
4-(diethylamino)-2-oxo-1,2-dihydroquinoline-3-carbonitrile;
4-(4,4-dimethylpiperidin-1-yl)-2-oxo-1,2-dihydroquinoline-3-carbonitrile; and
4-((2-(oxetan-3-yl)ethyl)amino)-2-oxo-1,2-dihydroquinoline-3-carbonitrile;
or a pharmaceutically acceptable salt thereof.

In some cases, as disclosed in U.S. Ser. No. 10/376,504, PDE9 inhibitor is selected from the group consisting of:

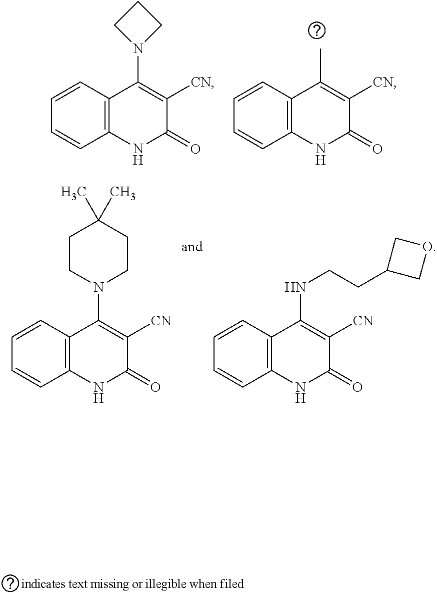

or a pharmaceutically acceptable salt thereof.

In some cases, as disclosed in WO2017019726, PDE9 inhibitor is represented by a compound of formula (AA-VII),

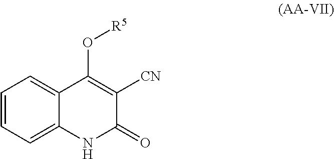

wherein:
R⁵ is selected from the group consisting of:
(1) —C₁₋₆alkyl, which is unsubstituted or substituted with hydroxy, oxetane, or allyl,
(2) -tetrahydropyranyl, and
(3) —C₆₋₇cycloalkyl; or a pharmaceutically acceptable salt thereof.

In some embodiments, PDE9 inhibitor is selected from the group consisting of:
4-(cyclohexyloxy)-2-oxo-1,2-dihydroquinoline-3-carbonitrile;
4-(cycloheptyloxy)-2-oxo-1,2-dihydroquinoline-3-carbonitrile;
2-oxo-4-tetrahydropyran-4-yloxy-1H-quinoline-3-carbonitrile;
2-oxo-4-(2-hydroxymethyl-2-methyl-1-butyloxy)-1H-quinoline-3-carbonitrile; and 4-((3-allyloxetan-3-yl)methoxy)-2-oxo-1,2-dihydroquinoline-3-carbonitrile;
or a pharmaceutically acceptable salt thereof.

In some cases, as disclosed in WO2017019724, PDE9 inhibitor is represented by a compound of formula (AA-VIII),

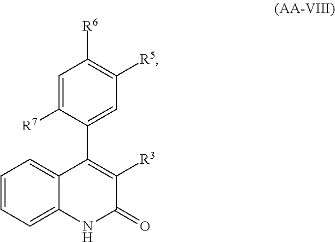

wherein
R³ is selected from the group consisting of:
   (1) —CN,
   (2) —CH₃, and
   (3) —CF₃;
R⁵, R⁶ and R⁷ are independently selected from the group consisting of.
   (1) hydrogen,
   (2) —C₁₋₆alkyl, and
   (3) fluoro,
or R⁵ and R together with the carbons to which they are attached are joined together to form a fused phenyl ring; or a pharmaceutically acceptable salt thereof.

In some embodiments, PDE9 inhibitor is selected from the group consisting of:
4-phenyl-2-oxo-1,2-dihydroquinoline-3-carbonitrile;
4-(4-Ethylphenyl)-3-methylquinolin-2(1H)-one;
2-oxo-4-[3-(pyridin-3-yl)pyrrolidin-1-yl]-1,2-dihydroquinoline-3-carbonitrile;
2-oxo-4-(p-tolyl)-1H-quinoline-3-carbonitrile;
4-(2-naphthyl)-2-oxo-1H-quinoline-3-carbonitrile; and
4-(2-fluoro-4-methyl-phenyl)-2-oxo-1H-quinoline-3-carbonitrile;
or a pharmaceutically acceptable salt thereof.

In some cases, as disclosed in U.S. Ser. No. 10/370,336, PDE9 inhibitor is represented by a compound of formula (AA-IX),

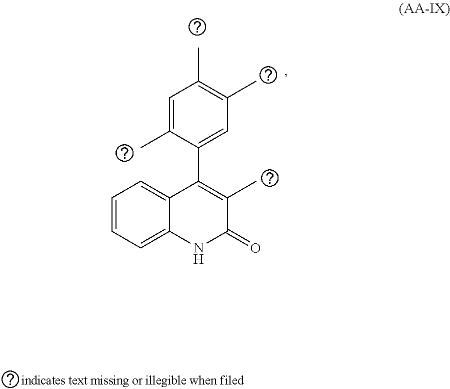

wherein
R⁵ is methyl or ethyl;
R⁶ is hydrogen; and
R⁷ is hydrogen or fluoro.

In some embodiments, PDE9 inhibitor is selected from the group consisting of:

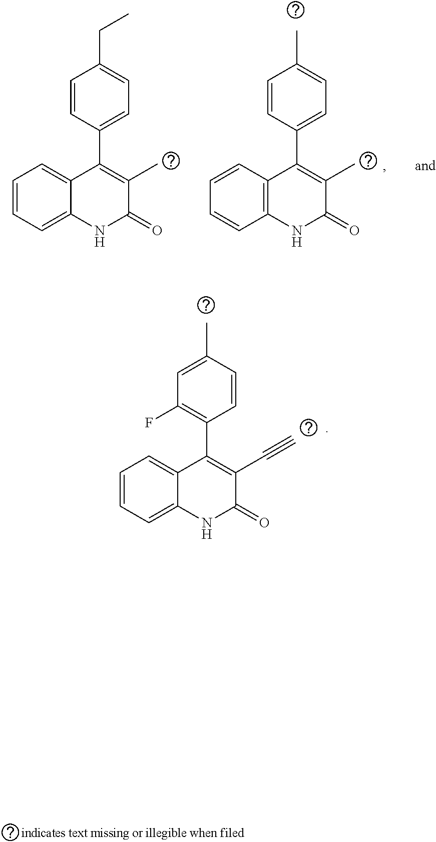 [SMILES: CCc1ccc(-c2c(C)c(=O)[nH]c3ccccc23)cc1 ; Cc1ccc(-c2c(C)c(=O)[nH]c3ccccc23)cc1 ; C#Cc1c(-c2ccc(C)cc2F)c2ccccc2[nH]c1=O]

In some cases, as disclosed in U.S. Pat. No. 9,617,269, PDE9 inhibitor is represented by a compound of formula (AA-X),

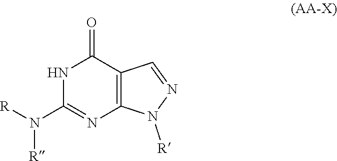

wherein
R′ is selected from the group consisting of isopropyl, cyclopentyl, cyclohexyl, and isobutyl; and
when R″═CH₃, R represents benzyl; and
when R″═H, R is selected from the group consisting of L-configured CHCH₃CONHR″′, D-configured CHCH₃CONHR″′, L-configured CH₂CONHR″′, D-configured CH₂CONHR″′, L-configured CH₂CH₂CONHR″′, D-configured CH₂CH₂CONHR″′, 3-methylpyridine, 1-phenylethyl, 1-(4-chlorophenyl)ethyl,

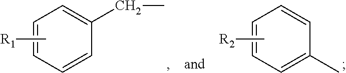

wherein R″′ is p-methoxyphenyl,
R₁ is selected from the group consisting of hydrogen, chlorine, methoxy, methyl, trifluoromethyl, dimethoxy, methylenedioxy, and dichlorine, and
R² is selected from the group consisting of hydrogen, methoxy, ethoxy, isopropoxy, methyl, dimethoxy, and 2-methyl-4-methoxy.

In some embodiments PDE9 inhibitor is selected from the group consisting of:

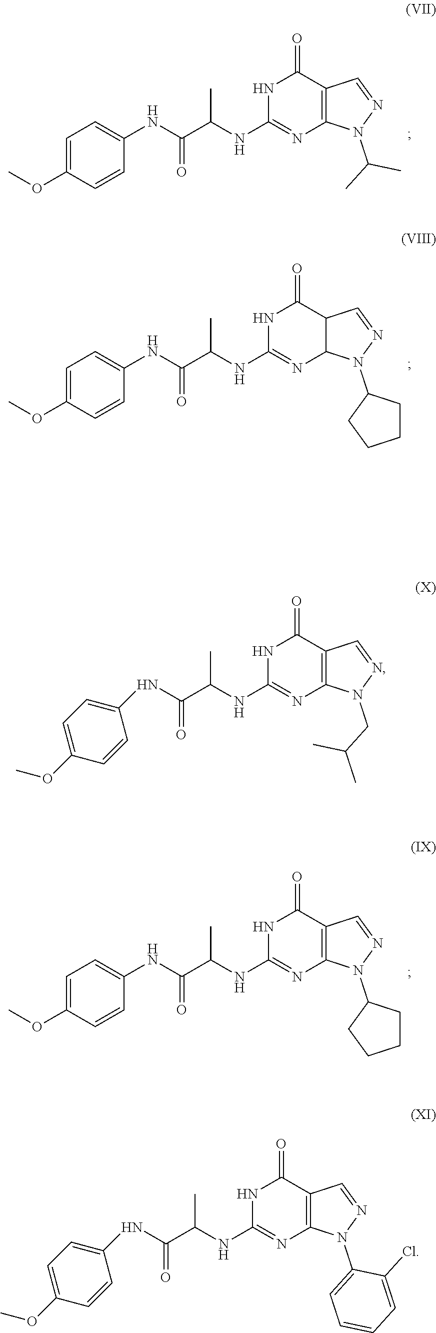

### Methods

The present disclosure provides a method of treating or preventing a disease in a subject, wherein the disease or disorder is treatable or preventable by administering an serotonergic psychedelic drug to the subject. In a certain aspect, the present disclosure provides a method of treating or preventing a disorder in a subject comprising by administering to the subject a therapeutically effective amount of a serotonergic psychedelic drug and a therapeutically effective amount of a PDE9 inhibitor. In some embodiments, the disorder comprises a neuropsychiatric disorder, cluster headache, or migraine headache. In some embodiments, the method disclosed herein is used to treat or prevent a neuropsychiatric disorder. In some embodiments, the method disclosed herein is used to treat or prevent cluster headache or migraine headache. In some embodiments, the PDE9 inhibitor enhances a therapeutic effect of the serotonergic psychedelic drug on the disorder to be treated. In some embodiments, the PDE9 inhibitor enhances efficacy of the serotonergic psychedelic drug on the disorder to be treated.

In some embodiments, the PDE9 inhibitor mitigates at least one undesirable biological activity of the serotonergic psychedelic drug in the subject. In certain embodiments, the PDE9 inhibitor reduces undesirable psychedelic effects of serotonergic psychedelic drug, rendering it more appropriate for widespread use. In some embodiments, the PDE9 inhibitor reduces undesirable psychedelic effects of serotonergic psychedelic drug and produces synergistic beneficial effects on behavior and neurophysiology. In other embodiments, the PDE-9 inhibitor prolongs the therapeutic effect(s) of the serotonergic psychedelic drug. In some embodiments, the PDE9 inhibitor reduces the abuse potential of the serotonergic psychedelic drug in the subject. In some embodiments of the present disclosure, PDE-9 inhibition blocks psychedelic effects of these drugs without attenuating their therapeutic effects.

The present disclosure further provides a method of preventing and/or minimizing abuse of a serotonergic psychedelic drug by a subject.

The present disclosure further provides a method of treating or preventing MDD, PTSD, OCD, and related psychiatric conditions in a subject.

In certain embodiments, the method comprises administering to the subject a therapeutically effective amount of a serotonergic psychedelic drug and a therapeutically effective amount of a PDE9 inhibitor.

In certain embodiments, the serotonergic psychedelic drug and the PDE9 inhibitor are co-administered to the subject. In other embodiments, serotonergic psychedelic drug and the PDE9 inhibitor are co-formulated as a pharmaceutical composition.

In certain embodiments, the PDE9 inhibitor is administered to the subject at least once before the serotonergic psychedelic drug is administered to the subject. In other embodiments, the PDE inhibitor is administered to the subject at least about 0.5 hours, about 1 hour, about 2 hours, or about 3 hours before the RAAD is administered to the subject.

In certain embodiments, the serotonergic psychedelic drug inhibitor is administered to the subject at least once before the PDE9 inhibitor is administered to the subject. In other embodiments, the serotonergic psychedelic drug is administered to the subject at least about 0.5 hours, about 1 hour, about 2 hours, or about 3 hours before the PDE9 inhibitor is administered to the subject.

In certain embodiments, the serotonergic psychedelic drug and the PDE9 inhibitor are independently administered to the subject by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous.

In certain embodiments, a pharmaceutical composition consisting of a serotonergic psychedelic drug together with a PDE9 inhibitor is administered to the subject by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous.

In certain embodiments, the subject is a mammal. In other embodiments, the mammal is human.

In one aspect, the present disclosure provides a method for treating depressive disorders and other neuropsychiatric conditions, such as (but not limited to) a major depressive disorder (MDD, including major depressive episode), a major depressive episode in bipolar disorder (bipolar depression), depressive episodes associated with bipolar I or II disorder (bipolar depression), a persistent depressive disorder (dysthymia), a disruptive mood dysregulation disorder, a premenstrual dysphoric disorder, a substance/medication-induced depressive disorder, a depressive disorder due to another medical condition, other specified depressive disorder, unspecified depressive disorder, an anxiety disorder, an obsessive-compulsive disorder, a posttraumatic stress disorder, an addictive disorder, treatment resistant depression (TRD), a generalized anxiety disorder (GAD), a post-traumatic stress disorder (PTSD), adjunctive treatment of major depression, eating disorders including anorexia and bulimia, depression associated with neurodegenerative disorders such as Parkinson's Disease, Alzheimer's Disease, dementias, and ALS, traumatic brain injury, suicidal thoughts and behaviors, chronic pain, a persistent depressive disorder, seasonal affective disorder (SAD), psychotic depression, peripartum (postpartum) depression, a premenstrual dysphoric disorder (PMDD), situational depression, and any combinations thereof. In certain embodiments, the subject suffering from the depressive disorder further suffers from an addictive disorder (i.e., the subject has comorbid depressive disorder and addictive disorder).

In certain embodiments, the subject in need of such treatment is administered a combination of a serotonergic psychedelic drug and a PDE9 inhibitor. In other embodiments, the serotonergic psychedelic drug and the PDE9 inhibitor are co-administered to the subject. In yet other embodiments, the serotonergic psychedelic drug and the PDE9 inhibitor are coformulated. In yet other embodiments, the methods of the present disclosure allow for reducing abuse potential of serotonergic psychedelic drugs by the subject. In yet other embodiments, the serotonergic psychedelic drug and the PDE9 inhibitor are the only active agents administered to the subject.

Non-limiting examples of serotonergic psychedelic drugs useful within the methods of the present disclosure include:
psilocybin (also known as [3-[2-(dimethylamino)ethyl]-1H-indol-4-yl]dihydrogen phosphate);
mescaline (also known as 2-(3,4,5-trimethoxyphenyl)ethanamine)
lysergic acid diethylamide (also known as LSD or (6aR,9R)—N,N-diethyl-7-methyl-6,6a,8,9-tetrahydro-4H-indolo[4,3-fg]quinoline-9-carboxamide);
ayahuasca or any preparations, fractions, or partially or fully purified fractions thereof;
dimethyltryptamine or DMT (also known as 2-(1H-indol-3-yl)-N,N-dimethylethanamine);
5-methoxy-dimethyltryptamine or 5-MeO-DMT (also known as 2-(5-methoxy-TH-indol-3-yl)-N,N-dimethylethanamine);
3,4-methoxy-diaminodextroamphetamine or MDMA (also known as N-methyl-1-(3,4-methylenedioxyphenyl)propan-2-amine or methylenedioxy-methylamfetamine)
any salt, solvate, enantiomer, tautomer and geometric isomer thereof, or any mixtures thereof.

In some embodiments, the psychedelic drug is selected from the group consisting of Psilocin, Psilocybin, Bufotenin, Baeocystin, Aeruginascin, 5-MeO-DMT, N,N-Dimethyltryptamine (DMT), 5-Bromo-DMT, N-Methyl-N-ethyltryptamine (MET), N-Methyl-N-isopropyltryptamine (MiPT), N-Methyl-N-propyltryptamine (MPT), N,N-Diethyltryptamine (DET), N-Ethyl-N-isopropyltryptamine (EiPT), N-Methyl-N-butyltryptamine (MBT), N-Propyl-N-isopropyltryptamine (PiPT), N,N-Dipropyltryptamine (DPT), N,N-Diisopropyltryptamine (DiPT), N,N-Diallyltryptamine (DALT), N,N-Dibutyltryptamine (DBT), N-Ethyltryptamine (NET), N-Methyltryptamine (NMT), Trimethyltryptamine (TMT), α-Methyltryptamine, α-Ethyltryptamine, α,N-DMT, α,N,N-Trimethyltryptamine, Ethocybin, 4-HO-MET, 4-HO-DET, 4-HO-MPT, 4-HO-MiPT, 4-HO-MALT, 4-HO-DPT, 4-HO-DiPT, 4-HO-DALT, 4-HO-DBT, 4-HO-DSBT, 4-HO-uMT, 4-HO-MPMI, 4-HO-TMT, 4-HO-1,N,N-TMT, 4-HO-5-MeO-DMT, 4-AcO-DMT, 4-AcO-MET, 4-AcO-MALT, 4-AcO-DET, 4-AcO-EiPT, 4-AcO-DPT, 4-AcO-DiPT, 4-AcO-DALT, 4-MeO-DMT, 4-MeO-MiPT, 5-MeO—N MT, 5-MeO-MET, 5-MeO-MPT, 5-MeO-MiPT, 5-MeO-DET, 5-MeO-Ei PT, 5-MeO-EPT, 5-MeO-Pi PT, 5-MeO-DPT, 5-MeO-DiPT, 5-MeO-DALT, 5-MeO-uMT, 5-MeO-2,N,N-TMT, 5-MeO-7,N,N-TMT, 5-MeO-α,N-DMT, 4-F-5-MeO-DMT, 5-Me-MiPT, 5-HO-DiPT, 2-α-DMT, 4-Me-uMT, 4-Me-αET, 7-Me-αET, 4,5-DHP-AMT, 4,5-DH P-DMT, 4,5-MDO-DMT, 4,5-MDO-DiPT, 5,6-MDO-DiPT, 5,6-MDO-MiPT, 5-Fluoro-uMT, 6-Fluoro-uMT, 6-Fluoro-DMT, N,N-Tetramethyl enetryptamine (Pyr-T), 4-HO-pyr-T, 5-MeO-pyr-T, RU-28306 (4,α-Methylene-N,N-DMT), O-4310 (6-Fluoro-1-Isopropyl-4-HO-DMT), CP-132,484 (4,5-DHP-1-Methyltryptamine), Dimemebfe (5-MeO-BFE), 5-MeO-DiBF, Ibogaine, Voacangine, Lysergic acid diethylamide (LSD), Lysergic acid amide (LSA), Lysergic acid diamide, N1-Methyl-lysergic acid diethylamide, 1-Propionyl-lysergic acid diethylamide, 1-cyclopropanoyl-d-lysergic acid diethylamide, 1-valeryl-D-lysergic acid diethylamide, 6-Allyl-6-nor-lysergic acid diethylamide, 6-Butyl-6-nor-lysergic acid diethylamide, 6-Ethyl-6-nor-lysergic acid diethylamide, 1-Propionyl-6-Ethyl-6-nor-lysergic acid diethylamide, 6-Propyl-6-nor-lysergic acid diethylamide, 6-Cyclopropyl-6-nor-lysergic acid diethylamide, 6-nor-Lysergic acid diethylamide, Lysergic acid ethylamide, Lysergic acid α-hydroxyethylamide, Lysergic acid 2-butyl amide, Lysergic acid 3-pentyl amide, Lysergic acid methyl ester, Lysergic acid 2,4-dimethylazetidide, Lysergic acid piperidine, N,N-Dimethyl-lysergamide, Methylisopropyllysergamide, N,N-Diallyllysergamide, N-Pyrrolidyllysergamide, N-Morpholinyllysergamide, 1-methyl-lysergic acid butanolamide, Lysergic acid β-propanolamide, Lysergic acid 1-butanolamide, Mescaline, Lophophine, Isomescaline, Cyclopropylmescaline, Thioisomescaline (2-TIM, 3-TIM, and 4-TIM), 4-Desoxymescaline, Jimscaline, Escaline, Metaescaline, Thiometaescaline (3-TME, 4-TME, and 5-TME), Trisescaline, Thiotrisescaline (3-T-TRIS and 4-T-TRIS), Symbescaline, Asymbescaline, Thiosymbescaline (3-TSB and 4-TSB), Phenescaline, Allylescaline, Methallylescaline, Proscaline, Isoproscaline, Metaproscaline, Thioproscaline, Buscaline, Thiobuscaline, α-ethylmescaline, Ariadne, Macromerine, MEPEA, TOM (2-TOM and 5-TOM), Bis-TOM, TOMSO (2-methoxy-4-methyl-5-methylsulfinylamphetamine), TOET (2-TOET and 5-TOET), BOH, BOM (13-Methoxy-mescaline), beta-D, 4-D, DME, F-2, F-22, FLEA, MDPH, MDMP, Propynyl, 2C family (2,5-dimethoxy, 4-substituted phenethylamines), βk-2C-B, 2C-B, 2CB-2EtO, 2CB-5EtO, 2CB-diEtO, 2C—B-FLY, 2C—B-BUTTERFLY, 2C—C, 2C-D, 2CD-2EtO, 2CD-diEtO, 2CD-5EtO, 2C-E, 2C-EF, 2C—F, 2C-G (2C-G-1, 2C-G-2, 2C-G-3, 2C-G-4, 2C-G-5, 2C-G-6, and 2C-G-N), 2C—H, 2C—I, 2CI-2EtO, 2C-iP, 2C—N, 2C—O, 2C—O-4, 2C—P, 2C-SE, 2C-T, 2CT-5EtO, 2C-T-2, 2CT-2-2EtO, 2CT-2-5EtO, 2CT-2-diEtO, 2C-T-4 (2C-T-4 and Ψ-2C-T-4), 2CT-4-2EtO, 2C-T-7, 2CT-7-2EtO, 2C-T-8, 2C-T-9, 2C-T-13, 2C-T-15, 2C-T-16, 2C-T-17, 2C-T-19, 2C-T-21, 2C-TFM, 2C—YN, BOB (I3-Methoxy-2C-B), BOD (I3-Methoxy-2C-D), BOHD (I3-Hydroxy-2C-D), HOT-2, HOT-7, HOT-17, Indane derivatives comprising 2CB-Ind, Benzocyclobutene derivatives comprising 2C—BCB (TCB-2), NBOMe derivatives comprising NBOMe-mescaline, 2C—H—NBOMe, 2C—C—NBOMe, 2CBCB—NBOMe, 2CBFly-NBOMe, 2C—B—NBOMe, 2C—I—NBOMe, 2C-TFM-NBOMe, 2C-D-NBOMe, 2C-G-NBOMe, 2C-E-NBOMe, 2C—P—NBOMe, 2C-iP-NBOMe, 2C—CN—NBOMe, 2C—N—NBOMe, 2C-T-NBOMe, 2C-T-4-NBOMe, 2C-T-7-NBOMe, and DMBMPP, NBOH derivatives comprising 2C—C—NBOH, 2C—B—NBOH, 2C—I—NBOH, and 2C—CN—NBOH, NBMD derivatives comprising 2C—I-NBMD, NBF derivatives comprising 2C—C—NBF, 2C—B—NBF, and 2C—I—NBF, 3C family (3,5-dimethoxy, 4-substituted amphetamines) comprising 3C-E, 3C—P, 3C-DFE, and 3C—BZ, DOx family (2,5-dimethoxy, 4-substituted amphetamines) comprising DOAM, DOB, Meta-DOB, Methyl-DOB, DOBU, DOC, DOEF, DOET, DOI, DOM, Ψ-DOM, DON, DOPR, SOiPR, DOT, Meta-DOT, Ortho-DOT, and DOTFM, DMCPA, DMMDA, DMMDA-2, 2,5-dimethoxy-3,4-dimethylamphetamine, 4-methyl-2,5-dimethoxymethamphetamine, 2,N-dimethyl-4,5-methylenedioxy amphetamine, Dimethoxyamphetamine (2,4-DMA, 2,5-DMA, and 3,4-DMA), Trimethoxy amphetamine (TMA-2, TMA-6), Tetramethoxyamphetamine, Br-DragonFLY, TFMFly, 2-Bromo-4,5-methylenedioxyamphetamine, 4-Bromo-3,5-dimethoxyamphetamine, EEE, EEM, EME, EMM, EDMA, EIDA, Ethyl-J, Methyl-J, Ethyl-K, Mehtyl-K, IDNNA, Iris, MDAI, MDMAI, MDAT, MDMAT, MDAL, MDBU, MDBZ, MDDM, MDIP, MDMEOET, MDMEO, MDOH, MDHOET, MDPL, MDCPK, MDPR, MEDA, MEM, Mehtyl-DMA, MMDA, MMDA-2, 5-Mehtyl-MDA, MEE, MME, MPM, DiFMDA, 5-APB, 6-APB, 5-APDB, 6-APDB, 5-MAPB, 5-MAPDB, 6-MAPDB, 6-MAPB, 6-EAPB, 5-EAPB, Para-Methoxyamphetamine, Paramethoxymethamphetamine, 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Benzoxazine, Efavirenz, Substituted methylenedioxy-phenethylamines (MDxx) comprising 3,4-methylenedioxymethamphetamine (MDMA), MDA, 2,3-MDA, 5-Methyl-MDA, MMDA, MDEA, MBDB, MDAL, MDBU, MDBZ, MDDM, MDIP, MDMEOET, MDMEO, MDOH, MDHOET, MDPL, MDCPM, MDPR, BDB, MMDA-2, DiFMDA, EIDA, Ethyl-K, Lophophine, Substituted amphetamines, EDMA, Para-Methoxyamphetamine (PMA), Paramethoxymethamphetamine (PMMA), 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Substituted cathinones, Methylone, Ethylone, Eutylone, Butylone, Pentylone, 4-Ethyl methcathinone, 3-Methylmethcathinone, Substituted benzofurans, 5-AP B, 6-AP B, 5-APDB, 6-APDB, 5-MAPB, 5-MAPDB, 6-MAPDB, 6-MAPB, 5-EAPB, 6-EAPB, 5-MBPB, MDAT, MDMAT, 6-CAT, Tetralinylaminopropane, Trifluoromethylaminoindane, Ethyltrifluoromethylaminoindane, 5-Iodo-2-aminoindane, MMAI, MDAI, MDMAI, Indanylaminopropane, Naphthylaminopropane, 4-chlorophenylisobutylamine, 4-Methylphenylisobutylamine, Ariadne, α-methyltryptamine, 5-MeO-αMT, α-ethyltryptamine, 4-Me-αET, 7-Me-αET, 5-MeO-αET, 5-MeO-MiPT, Δ⁹-THC, CBD, CBN, THCV, (C6)-CP 47,497, (C9)-CP 47,497, 1-Butyl-3-(2-methoxybenzoyl)indole, 1-Butyl-3-(4-methoxybenzoyl)indole, 1-Pentyl-3-(2-methoxybenzoyl)indole, 2-Isopropyl-5-methyl-1-(2,6-di hydroxy-4-nonylphenyl)cyclohex-1-ene, 4-HTMPIPO, 4-Nonylphenylboronic acid, 5Br—U R-144, 5Cl-APINACA, 5Cl—U R-144, 5F-3-pyridinoylindole, 5F-AB-FUPPYCA, 5F-ADB-PINACA, 5F-ADBICA, 5F-ADB, 5F-AMB, 5F-APINACA, 5F-CUMYL-PINACA, 5F-EMB-PINACA, 5F-NNE1, 5F—PB-22, 5F—PCN, 5F-PY-PICA, 5F—PY—P INACA, 5F-SDB-006, HHC, A-796,260, A-834,735, A-836,339, A-955,840, A-40174, A-41988, A-42574, AB-001, AB-CH FU PYCA, AB-CHMFUPPYCA, AB-CHMINACA, AB-FUBICA, AB-FUBINACA 2-fluorobenzyl isomer, AB-FUBINACA, AB-PICA, AB-PINACA, Abnormal cannabidiol, ADAMANTYL-THPINACA, ADB-CHMINACA, ADB-FUBICA, ADB-FUBINACA, ADB-PINACA, ADBICA, ADS B—FU B-187, Ajulemic acid, AM-087, AM-411, AM-630, AM-679, AM-694, AM-855, AM-883, AM-905, AM-906, AM-919, AM-926, AM-938, AM-1220, AM-1221, AM-1235, AM-1241, AM-1248, AM-1346, AM-1387, AM-1714, AM-2201, AM-2232, AM-2233, AM-2389, AM-4030, AM-4113, AM-6527, AM-6545, AM-251, AM-281, AM-404, AMB-CHMINACA, AMB-FUBINACA, AMG-1, AMG-3, AMG-36, AMG-41, APICA, APINACA, APP-FUBINACA, Arachidonoyl SEROTONIN, ACEA, ACPA, Arvanil, AZ-11713908, BAY 38-7271, BAY 59-3074, BIM-018, Biochanin A, BML-190, Nabidrox (Canbisol), Cannabicyclohexanol, Cannabipiperidiethanone, CAY-10401, CAY-10429, CAY-10508, CB-13, CB-25, CB-52, CB-86, CB-86, CBS-0550, CP 47,497, CP 55,244, CP 55,940, CUMYL-5F-PICA, CUMYL-BICA, CUMYL-PICA, CUMYL-PINACA, CUMYL-THPINACA, Dexanabinol, Dimethylheptylpyran, Drinabant, Dronabinol, EAM-2201, EMB-FUBINACA, FAB-144, FDU-NNE1, FDU-PB-22, FUB-144, FUB-APINACA, FUB-JWH-018, FUB—PB-22, FUBIMINA, Genistein, GW-405,833, GW-842,166X, Hemopressin, HU-210, HU-243, HU-308, HU-320, HU-331, HU-336, HU-345, HU-910, Ibipinabant, IDFP, JNJ 1661010, JNJ 1661010, JTE-907, JTE 7-31, JWH-007, JWH-015, JWH-018, JWH-019, JWH-030, JWH-051, JWH-073, JWH-081, JWH-098, JWH-116, JWH-122, JWH-133, JWH-139, JWH-147, JWH-149, JWH-161, JWH-164, JWH-167, JWH-175, JWH-176, JWH-182, JWH-184, JWH-185, JWH-192, JWH-193, JWH-194, JWH-195, JWH-196, JWH-197, JWH-198, JWH-199, JWH-200, JWH-203, JWH-210, JWH-229, JWH-249, JWH-250, JWH-251, JWH-302, JWH-307, JWH-359, JWH-369, JWH-370, JWH-398, JWH-424, JZL184, JZL195, Kaempferol, KM-233, L-759,633, L-759,656, LASSBio-881, LBP-1, Leelamine, Levonantradol, LH-21, LY-320,135, LY-2183240, NAM-2201, MDM-2201, MDA-7, MDA-19, MDA-77, MDMB-CHMICA, MDMB-CHMINACA, MDMB-FUBINACA, Menabitan, MEPIRAPIM, Methanandamide, MJ-15, MK-9470, MMB-2201, MN-18, MN-25, Nabazenil, Nabilone, Nabitan, Naboctate, NESS-0327, NESS-040C5, NIDA-41020, NM-2201, NMP-7, NNE1, Nonabine O-224, O-581, O-585, O-606, O-689, O-774, O-806, O-823, O-889, O-1057, O-1125, O-1184, O-1191, O-1238, O-1248, O-1269, O-1270, O-1376, O-1399, O-1422, O-1601, O-1602, O-1624, O-1656, O-1657, O-1660, O-1812, O-1860, O-1861, O-1871, O-1918, O-2048, O-2050, O-2093, O-2113, O-2220, O-2365, O-2372, O-2373, O-2383, O-2426, O-2484, O-2545, O-2654, O-2694, O-2715, O-2716, O-3223, O-3226, Oleoylethanolamide, Olvanil, Org 27569, Org 27759, Org 2831, Org 28611, Org 29647, Otenabant, Palmitoylethanolamide, Parahexyl, PF-03550096, PF-04457845, PF-622, PF-750, PF-3845, PF-514273, PHOP, PipISB, Pimabine, Pravadoline, Pregnenolone, PSB—SB-487, PSB—SB-1202, PTI-1, PTI-2, PX-1, PX-2, PX-3, QUCHIC, QUPIC, RCS-4, RCS-8, Rimonabant, Rosonabant, RTI-371, S-444,823, SDB-006, SER-601, SPA-229, SR-144,528, STS-135, Surinabant, Taranabant, Tedalinab, THC-O-acetate, THC-O-phosphate, THJ-018, THJ-2201, Tinabinol, TM-38837, UR-144, URB-447, URB-447, URB-597, URB-602, URB-754, VCHSR, VDM-11, VSN-16, WIN 54,461, WIN 55,212-2, WIN 56,098, XLR-11, Yangonin, Harmaline, harmala alkaloids, other beta-carbolines, active constituents of ayahuasca, Salvinorin A, Salvinorin B methoxymethyl ether, Salvinorin B ethoxymethyl ether, Piperazines, pFPP, TFMPP, Myristicin, Elemicin, Cryogenine (Vertine), Atropine, Scopolamine, Hyoscyamine, Ibotenic acid, Muscimol, TiHKAL, 2-Me-DET, 4-HO-DBT, 4-HO-DET, 4-HO-DiPT, 4-HO-EPT, 4-HO-McPT, 4-HO-MET, 4-HO-MiPT, 4-HO-MPMI, 4-HO-MPT, 4-HO-uMT, 4-Me-uMT, 4-MeO-MiPT, 4-PrO-DMT, 4,5-MDO-DiPT, 4,5-MDO-DMT, 5-Ethoxy-uMT, 5-Fluoro-AET, 5-Fluoro-DET, 5-Fluoro-DMT, 5-Fluoro-EPT, 5-Fluoro-MET, 5-MeO-AET, 5-MeO-αMT, 5-MeO-DALT, 5-MeO-DET, 5-MeO-DPT, 5-MeO-EiPT, 5-MeO-MALT, 5-MeO-MiPT, 5-MeO-MPMI, 5-MeO-pyr-T, 5-MeS-DMT, 5-MeO-2-TMT, 5-TFM-DMT, 5-TFMO-DMT, 5,6-MDO-DiPT, 5,6-MDO-DMT, 5,6-MDO-MiPT, 5,6-MeO-MiPT, 5,N,N-TMT, 5F-MPMI, 6-Fluoro-DET, 6-Fluoro-DMT, 6-MeO-THH, 7-Chloro-AMT, 7-F-5-MeO-MET, 4-Acetoxy-DET, 4-Acetoxy-DiPT, 4-Acetoxy-MET, 4-Acetoxy-MiPT, O-Acetylbufotenine, O-Acetylpsilocin, Aeruginascin, Alpha,N-DMT, Alpha,N,O-TMS, Baeocystin, 5-Bromo-DMT, Bufotenin, 5-Chloro-αMT, DALT, Dibutyltryptamine, Diethyltryptamine, Diisopropyltryptamine, 5,N-Dimethyl-N-isopropyltryptamine, Dimethyltryptamine, N,N-Dimethyltryptamine, Dipropyltryptamine, 2,α-Dimethyltryptamine, Ethocybin, Ethylisopropyltryptamine, A-Ethyltryptamine, 5-Fluoro-AMT, 4-Fluoro-5-methoxy-DMT, FT-104, 5-MeO-DMT, 5-Methoxy-N,N-diisopropyltryptamine, 4-Methyl-α-ethyltryptamine, N-Methyl-N-ethyltryptamine, Methylbutyltryptamine, Methylisopropyltryptamine, Alpha-Methylserotonin, Alpha-Methyltryptamine, N-Methyltryptamine, MPMI, Norbaeocystin, Propylisopropyltryptamine, 2,N,N-TMT, A,N,N-Trimethyltryptamine, Ketamine, Esketamine, Arketamine, Mitragynine, Yohimbine, and any combinations thereof.

In certain embodiments, the serotonergic psychedelic drug is psilocybin, or a salt, solvate, enantiomer, or diastereomer thereof. In certain embodiments, the serotonergic psychedelic drug is mescaline, or a salt, solvate, enantiomer, or diastereomer thereof. In certain embodiments, the serotonergic psychedelic drug is lysergic acid diethylamide, or a salt, solvate, enantiomer, or diastereomer thereof. In certain embodiments, the serotonergic psychedelic drug is ayahuasca, or a preparation, fraction, or partially purified fraction thereof. In certain embodiments, the serotonergic psychedelic drug is dimethyltriptamine, or a salt, solvate, enantiomer, or diastereomer thereof. In certain embodiments, the serotonergic psychedelic drug is 5-methoxy-dimethyltryptamine, or a salt, solvate, enantiomer, or diastereomer thereof. In certain embodiments, the serotonergic psychedelic drug is MDMA, or a salt, solvate, enantiomer, or diastereomer thereof. In some embodiments, the serotonergic psychedelic drug is a tryptamine or an ergoline. In some embodiments, the serotonergic psychedelic drug is a tryptamine, or a salt, solvate, enantiomer, or diastereomer thereof. In some embodiments, the serotonergic psychedelic drug is an ergoline, or a salt, solvate, enantiomer, or diastereomer thereof.

In certain embodiments, the serotonergic psychedelic drug is not psilocybin, or a salt, solvate, enantiomer, or diastereomer thereof. In certain embodiments, the serotonergic psychedelic drug is not mescaline, or a salt, solvate, enantiomer, or diastereomer thereof. In certain embodiments, the serotonergic psychedelic drug is not lysergic acid diethylamide, or a salt, solvate, enantiomer, or diastereomer thereof. In certain embodiments, the serotonergic psychedelic drug is not ayahuasca, or a preparation, fraction, or partially purified fraction thereof. In certain embodiments, the serotonergic psychedelic drug is not dimethyltriptamine, or a salt, solvate, enantiomer, or diastereomer thereof. In certain embodiments, the serotonergic psychedelic drug is not 5-methoxy-dimethyltryptamine, or a salt, solvate, enantiomer, or diastereomer thereof. In certain embodiments, the serotonergic psychedelic drug is not MDMA, or a salt, solvate, enantiomer, or diastereomer thereof.

In certain embodiments, the PDE9 inhibitor is aminophylline, or a derivative thereof, or a salt, solvate, enantiomer or diastereomer thereof. In certain embodiments, the PDE9 inhibitor is pentoxifylline, or a derivative thereof, or a salt, solvate, enantiomer or diastereomer thereof. In certain embodiments, the PDE9 inhibitor is theobromine, or a derivative thereof, or a salt, solvate, enantiomer or diastereomer thereof. In certain embodiments, the PDE9 inhibitor is paraxanthine, or a derivative thereof, or a salt, solvate, enantiomer or diastereomer thereof. In certain embodiments, the PDE9 inhibitor is PF-4447913, or a derivative thereof, or a salt, solvate, enantiomer or diastereomer thereof. In certain embodiments, the PDE9 inhibitor is PF-4447943, or a derivative thereof, or a salt, solvate, enantiomer or diastereomer thereof. In certain embodiments, the PDE9 inhibitor is PF-4447953, or a derivative thereof, or a salt, solvate, enantiomer or diastereomer thereof. In certain embodiments, the PDE9 inhibitor is BAY 73-6691, or a derivative thereof, or a salt, solvate, enantiomer or diastereomer thereof. In certain embodiments, the PDE inhibitor is WYQ-C36D, or a derivative thereof, or a salt, solvate, enantiomer or diastereomer thereof. In certain embodiments, the PDE9 inhibitor is E 2027, or a derivative thereof, or a salt, solvate, enantiomer or diastereomer thereof. In certain embodiments, the PDE9 inhibitor is CRD 773, or a derivative thereof, or a salt, solvate, enantiomer or diastereomer thereof. In certain embodiments, the PDE9 inhibitor is BI 409306, or a derivative thereof, or a salt, solvate, enantiomer or diastereomer thereof. In certain embodiments, the PDE9 inhibitor is CRD 740, or a derivative thereof, or a salt, solvate, enantiomer or diastereomer thereof. In certain embodiments, the PDE9 inhibitor is TT 00920, or a derivative thereof, or a salt, solvate, enantiomer or diastereomer thereof. In certain embodiments, the PDE9 inhibitor is BAY 7081, or a derivative thereof, or a salt, solvate, enantiomer or diastereomer thereof. In certain embodiments, the PDE9 inhibitor is FRM 16606, or a derivative thereof, or a salt, solvate, enantiomer or diastereomer thereof. In certain embodiments, the PDE9 inhibitor is HUC1-288, or a derivative thereof, or a salt, solvate, enantiomer or diastereomer thereof.

In certain embodiments, the PDE9 inhibitor is not aminophylline, or a derivative thereof, or a salt, solvate, enantiomer or diastereomer thereof. In certain embodiments, the PDE9 inhibitor is not pentoxifylline, or a derivative thereof, or a salt, solvate, enantiomer or diastereomer thereof. In certain embodiments, the PDE9 inhibitor is not theobromine, or a derivative thereof, or a salt, solvate, enantiomer or diastereomer thereof. In certain embodiments, the PDE9 inhibitor is not paraxanthine, or a derivative thereof, or a salt, solvate, enantiomer or diastereomer thereof. In certain embodiments, the PDE9 inhibitor is not PF-4447913, or a derivative thereof, or a salt, solvate, enantiomer or diastereomer thereof. In certain embodiments, the PDE9 inhibitor is not PF-4447943, or a derivative thereof, or a salt, solvate, enantiomer or diastereomer thereof. In certain embodiments, the PDE9 inhibitor is not PF-4447953, or a derivative thereof, or a salt, solvate, enantiomer or diastereomer thereof. In certain embodiments, the PDE9 inhibitor is not BAY 73-6691, or a derivative thereof, or a salt, solvate, enantiomer or diastereomer thereof. In certain embodiments, the PDE9 inhibitor is not WYQ-C₃₆D, or a derivative thereof, or a salt, solvate, enantiomer or diastereomer thereof. In certain embodiments, the PDE9 inhibitor is not E 2027, or a derivative thereof, or a salt, solvate, enantiomer or diastereomer thereof. In certain embodiments, the PDE9 inhibitor is not CRD 773, or a derivative thereof, or a salt, solvate, enantiomer or diastereomer thereof. In certain embodiments, the PDE9 inhibitor is not CRD 740, or a derivative thereof, or a salt, solvate, enantiomer or diastereomer thereof. In certain embodiments, the PDE9 inhibitor is not TT 00920, or a derivative thereof, or a salt, solvate, enantiomer or diastereomer thereof. In certain embodiments, the PDE9 inhibitor is not BAY 7081, or a derivative thereof, or a salt, solvate, enantiomer or diastereomer thereof. In certain embodiments, the PDE9 inhibitor is not FRM 16606, or a derivative thereof, or a salt, solvate, enantiomer or diastereomer thereof. In certain embodiments, the PDE9 inhibitor is not HUC1-288, or a derivative thereof, or a salt, solvate, enantiomer or diastereomer thereof.

In some embodiments, the method comprises administration of a therapeutically effective amount of a tryptamine, or a derivative thereof, or a salt, solvate, enantiomer, or diastereomer thereof to a subject in need. In some embodiments, the method comprises administration of a therapeutically effective amount of an ergoline, or a derivative thereof, or a salt, solvate, enantiomer, or diastereomer thereof to a subject in need. In some embodiments, the method comprises administration of a therapeutically effective amount of psilocybin, or a derivative thereof, or a salt, solvate, enantiomer, or diastereomer thereof to a subject in need. In some embodiments, the method comprises administration of a therapeutically effective amount of PF-04447943, or a derivative thereof, or a salt, solvate, enantiomer or diastereomer thereof to a subject in need. In some embodiments, the method comprises administration of a therapeutically effective amount of psilocybin and PF-04447943 to a subject in need. In some embodiments, the method comprises administration of a therapeutically effective amount of psilocybin, or a derivative thereof, or a salt, solvate, enantiomer or diastereomer thereof and PF-04447943, or a derivative thereof, or a salt, solvate, enantiomer or diastereomer thereof to a subject in need.

The compounds of the present disclosure may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the present disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ¹³C, ¹⁴C, ³⁵Cl, ¹⁶F, ¹²³I, ¹²⁵I, ¹N, ¹⁵N, ¹⁵O, ¹⁷O, ¹⁸O, ³²P, and ³³S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed. In certain embodiments, the compounds described herein are labeled by other means, including, but not limited to, the use of chromophores or fluorescent moieties, bioluminescent labels, or chemiluminescent labels.

### Salts

The compounds described herein can form salts with acids and/or bases, and such salts are included in the present disclosure. In certain embodiments, the salts are pharmaceutically acceptable salts. The term “salts” embraces addition salts of free acids and/or bases that are useful within the methods of the present disclosure. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present disclosure, such as for example utility in the process of synthesis, purification or formulation of compounds useful within the methods of the present disclosure.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the present disclosure include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (also known as N-methylglucamine) and procaine. All of these salts may be prepared from the corresponding compound by reacting, for example, the appropriate acid or base with the compound.

### Compositions

The present disclosure includes pharmaceutical compositions comprising at least one pharmaceutically acceptable carrier and one or more pharmaceutically active agents contemplated herein. The pharmaceutical compositions described herein may be prepared by any method known or hereafter developed in the art of pharmacology. In general, such preparatory methods include bringing the active ingredient into association with a carrier or one or more other accessory ingredients, and then, if necessary or desirable, shaping or packaging the product into a desired single- or multi-dose unit.

In certain embodiments, the serotonergic psychedelic drug and the PDE9 inhibitor are coformulated in a pharmaceutical composition. In other embodiments, the serotonergic psychedelic drug and the PDE9 inhibitor are coformulated in a way whereby physical separation of the serotonergic psychedelic drug and the PDE9 inhibitor and/or immunosuppressant is not possible and/or feasible. In yet other embodiments, separation of the serotonergic psychedelic drug and the PDE9 inhibitor requires chemical purification (using extractive procedures, chromatographic separation and the like). In yet other embodiments, separation of the serotonergic psychedelic drug and the PDE9 inhibitor requires denaturation and/or destruction of the pharmaceutical composition.

In certain embodiments, the pharmaceutical compositions of the present disclosure comprise a serotonergic psychedelic drug and a PDE9 inhibitor, wherein the serotonergic psychedelic drug and the PDE9 inhibitor are present in amounts whereby administration of the pharmaceutical compositions to a subject treats in the subject a disease or disorder that is treatable or preventable with serotonergic psychedelic drug; and the PDE9 inhibitor prolongs at least one biological effect of the serotonergic psychedelic drug in the subject. In certain embodiments, the pharmaceutical compositions of the present disclosure comprise a serotonergic psychedelic drug and a PDE9 inhibitor, wherein the serotonergic psychedelic drug and the PDE9 inhibitor are present in amounts whereby administration of the pharmaceutical compositions to a subject treats in the subject a disease or disorder that is treatable or preventable with serotonergic psychedelic drug; and the PDE9 inhibitor mitigates at least one undesirable biological effect of the serotonergic psychedelic drug in the subject. In certain embodiments, the pharmaceutical compositions of the present disclosure comprise a serotonergic psychedelic drug and a PDE9 inhibitor, wherein the serotonergic psychedelic drug and the PDE9 inhibitor are present in amounts whereby administration of the pharmaceutical compositions to a subject treats in the subject a disease or disorder that is treatable or preventable with serotonergic psychedelic drug; and the PDE9 inhibitor augments at least one therapeutic biological effect of the serotonergic psychedelic drug in the subject.

In certain embodiments, the pharmaceutical compositions of the present disclosure, or any of the active agents contemplated in the present disclosure (separately and/or in combination), are administered to the subject in need thereof using any known and applicable route of administration, such as for example, (intra)nasal, inhalational, topical, oral, buccal, rectal, pleural, peritoneal, vaginal, intramuscular, subcutaneous, transdermal, epidural, intratracheal, otic, intraocular, intrathecal, and intravenous. In certain embodiments, the pharmaceutical compositions of the present disclosure, or any of the active agents contemplated in the present disclosure (separately and/or in combination), are administered to the subject in need thereof (intra)nasally. In yet other embodiments, the pharmaceutical compositions of the present disclosure, or any of the active agents contemplated in the present disclosure (separately and/or in combination), are administered to the subject using a transdermal patch.

Contemplated modes of administration of compounds and/or compositions useful within the present disclosure, including (intra)nasal delivery, are recited in the following applications, which are incorporated herein in their entireties by reference: US20040059003, US20040138298, US20040166067, US20070287753, US20080041367, US20080153879, US20120270903, US20120277267, US20140171514, US20140256821, US20140274981, US20140275276, US20140275277, US20140275278, US20150056308, US20150196501, US20150283123, US20160000733, US20160175266, EP1103256, and DE102007009888.

In certain embodiments, the dose of the serotonergic psychedelic drug administered (intra)nasally, intravenously, intramuscularly and/or orally to the subject ranges from about 0.15 mg/kg to about 10 mg/kg. For example, the dose of the serotonergic psychedelic drug administered (intra)nasally, intravenously, intramuscularly and/or orally to the subject is selected from the group consisting of about 0.15 mg/kg, 0.2 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, and any fraction or multiple thereof.

In certain embodiments, the amount of serotonergic psychedelic drug administered (intra)nasally, intravenously, intramuscularly and/or orally to the subject ranges from about 1 mg to about 200 mg. For example, the amount of serotonergic psychedelic drug administered (intra)nasally, intravenously, intramuscularly and/or orally to the subject is selected from the group consisting of about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, and any fraction or multiple thereof.

In certain embodiments, the dose of the PDE9 inhibitor (such as, but not limited to, PF-4447913) administered (intra)nasally, intravenously, intramuscularly and/or orally to the subject ranges from about 0.5 mg to about 40 mg. For example, the dose of PDE9 inhibitor administered intravenously to the subject is selected from the group consisting of about 0.5 mg, 1 mg, 2 mg, 3 mg, 4 mg, 5 mg, 6 mg, 7 mg, 8 mg, 9 mg, 10 mg, 15 mg, 20 mg, 30 mg, 40 mg, and any fraction or multiple thereof.

In some embodiments, the pharmaceutical composition of the present disclosure comprises a tryptamine, or a salt, solvate, enantiomer, or diastereomer thereof. In some embodiments, the pharmaceutical composition of the present disclosure comprises an ergoline, or a salt, solvate, enantiomer, or diastereomer thereof. In some embodiments, the pharmaceutical composition of the present disclosure comprises psilocybin, or a salt, solvate, enantiomer, or diastereomer thereof. In some embodiments, the pharmaceutical composition of the present disclosure comprises PF-04447943, or a salt, solvate, enantiomer, or diastereomer thereof. In some embodiments, the pharmaceutical composition of the present disclosure comprises psilocybin and PF-04447943. In some embodiments, the pharmaceutical composition of the present disclosure comprises psilocybin, or a salt, solvate, enantiomer, or diastereomer thereof and PF-04447943, or a salt, solvate, enantiomer, or diastereomer thereof.

### Administration/Dosage/Formulations

The therapeutic formulations may be administered to the subject either prior to or after the onset of a disease or disorder contemplated in the present disclosure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated in the present disclosure. An effective amount of the therapeutic compound or composition necessary to achieve a therapeutic effect may vary according to factors such as the state of the disease or disorder in the patient; the age, sex, and weight of the patient; and the ability of the therapeutic compound to treat a disease or disorder contemplated in the present disclosure. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present disclosure is from about 1 and 5,000 mg/kg of body weight/per day. The pharmaceutical compositions useful for practicing the present disclosure may be administered to deliver a dose of from 1 ng/kg/day and 100 mg/kg/day. In certain embodiments, the present disclosure envisions administration of a dose which results in a concentration of the compound of the present disclosure from 1 μM and 10 pM in a mammal. One of ordinary skill in the art would be able to study the relevant factors and make the determination regarding the effective amount of the therapeutic compound without undue experimentation.

Actual dosage levels of the active ingredients in the pharmaceutical compositions of this present disclosure may be varied so as to obtain an amount of the active ingredient that is effective to achieve the desired therapeutic response for a particular patient, composition, and mode of administration, without being toxic to the patient.

The selected dosage level can depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician, having ordinary skill in the art may readily determine and prescribe the effective amount of the pharmaceutical composition required. For example, the physician could start doses of the compounds of the present disclosure employed in the pharmaceutical composition at levels lower than that required in order to achieve the desired therapeutic effect and gradually increase the dosage until the desired effect is achieved.

In embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the present disclosure.

In some embodiments, the composition of the present disclosure comprises a tryptamine, or a salt, solvate, enantiomer, or diastereomer thereof. In some embodiments, the composition of the present disclosure comprises an ergoline, or a salt, solvate, enantiomer, or diastereomer thereof. In some embodiments, the composition of the present disclosure comprises psilocybin, or a salt, solvate, enantiomer, or diastereomer thereof. In some embodiments, the composition of the present disclosure comprises PF-04447943, or a salt, solvate, enantiomer, or diastereomer thereof. In some embodiments, the composition of the present disclosure comprises psilocybin and PF-04447943. In some embodiments, the composition of the present disclosure comprises psilocybin, or a salt, solvate, enantiomer, or diastereomer thereof and PF-04447943, or a salt, solvate, enantiomer, or diastereomer thereof.

In some embodiments, the therapeutic formulation of the present disclosure comprises a tryptamine, or a salt, solvate, enantiomer, or diastereomer thereof. In some embodiments, the therapeutic formulation of the present disclosure comprises an ergoline, or a salt, solvate, enantiomer, or diastereomer thereof. In some embodiments, the therapeutic formulation of the present disclosure comprises psilocybin, or a salt, solvate, enantiomer, or diastereomer thereof. In some embodiments, the therapeutic formulation of the present disclosure comprises PF-04447943, or a salt, solvate, enantiomer, or diastereomer thereof. In some embodiments, the therapeutic formulation of the present disclosure comprises psilocybin and PF-04447943. In some embodiments, the composition of the present disclosure comprises psilocybin, or a salt, solvate, enantiomer, or diastereomer thereof and PF-04447943, or a salt, solvate, enantiomer, or diastereomer thereof.

In one embodiment, the compositions of the present disclosure are formulated using one or more pharmaceutically acceptable excipients or carriers. In one embodiment, the pharmaceutical compositions of the present disclosure comprise a therapeutically effective amount of a compound of the present disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the present disclosure are administered to the patient in dosages that range from one to five times per day or more. In another embodiment, the compositions of the present disclosure are administered to the patient in range of dosages that include, but are not limited to, once every day, every two, days, every three days to once a week, and once every two weeks.

Compounds of the present disclosure for administration may be in the range of from about 1 g to about 10,000 mg, about 20 g to about 9,500 mg, about 40 pg to about 9,000 mg, about 75 pg to about 8,500 mg, about 150 pg to about 7,500 mg, about 200 pg to about 7,000 mg, about 3050 pg to about 6,000 mg, about 500 pg to about 5,000 mg, about 750 pg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the present disclosure is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of a compound of the present disclosure is at most about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of a PDE9 inhibitor of the present disclosure is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of a PDE9 inhibitor of the present disclosure is at most about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of a serotonergic psychedelic drug of the present disclosure is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of a serotonergic psychedelic drug of the present disclosure is at most about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of PF-4447943 of the present disclosure is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of PF-4447943 of the present disclosure is at most about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of psilocybin of the present disclosure is at least about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP. In some embodiments, the dose of psilocybin of the present disclosure is at most about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 120, 140, 160, 180, 200, 300, 400, 500, 600, 700, 800, 900, 1000, 1500, 2000, 2500, or 3000 mg/kg IP.

In some embodiments, the dose of a compound of the present disclosure is from about 1 mg and about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present disclosure is directed to a packaged pharmaceutical composition comprising a container holding a therapeutically effective amount of a compound of the present disclosure, alone or in combination with a second pharmaceutical agent; and instructions for using the compound to treat, prevent, or reduce one or more symptoms of a disease or disorder contemplated in the present disclosure.

Formulations may be employed in admixtures with conventional excipients, e.g., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined, where desired, with other active agents.

Routes of administration of any of the compositions of the present disclosure include oral, nasal, rectal, intravaginal, parenteral, buccal, sublingual or topical. The compounds for use in the present disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration, microencapsulated formulations, and the like. It should be understood that the formulations and compositions that would be useful in the present disclosure are not limited to the particular formulations and compositions that are described herein.

### Oral Administration

For oral application, suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutical excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. Parenteral administration is contemplated to include, but is not limited to, subcutaneous, intravenous, intraperitoneal, intramuscular, intrastemal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a nontoxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this present disclosure include dosage forms as described in U.S. Pat. Nos. 6,340,475; 6,488,962; 6,451,808; 5,972,389; 5,582,837; and 5,007,790. Additional dosage forms of this present disclosure also include dosage forms as described in U.S. Patent Applications Nos. 20030147952; 20030104062; 20030104053; 20030044466; 20030039688; and 20020051820. Additional dosage forms of this present disclosure also include dosage forms as described in U.S. Pat. Nos. 7,976,870; 7,438,927; US20030091630; U.S. Pat. Nos. 6,723,340; 7,413,751; US20030031711; U.S. Pat. Nos. 6,451,808; 6,488,962; 7,736,667; 7,405,238; US20020051820; U.S. Pat. No. 5,972,389; WO 97/47285; WO 93/18755; and U.S. Pat. No. 5,007,790.

### Controlled Release Formulations and Drug Delivery Systems

In one embodiment, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the present disclosure may be administered in the form of microparticles, for example, by injection or in the form of wafers or discs by implantation.

In one embodiment of the present disclosure, the compounds of the present disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of a disease or disorder contemplated in the present disclosure. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the present disclosure is optionally given continuously; alternatively, the dose of drug being administered is temporarily reduced or temporarily suspended for a certain length of time (i.e. a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

Toxicity and therapeutic efficacy of such therapeutic regimens are optionally determined in cell cultures or experimental animals, including, but not limited to, the determination of the LD 50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

### Combination Therapies

In certain embodiments, the compounds of the present disclosure are useful in the methods of the present disclosure in combination with at least one additional agent useful for treating or preventing a disease or disorder contemplated herein in a mammal in need thereof. This additional agent may comprise compounds identified herein or compounds, e.g., commercially available compounds, known to treat, prevent or reduce the symptoms of a disease or disorder contemplated herein.

A synergistic effect may be calculated, for example, using suitable methods such as, for example, the Sigmoid-Emax equation (Holford & Schemer, 1981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Kits

The present disclosure includes a kit comprising at least one composition of the present disclosure, an applicator, and an instructional material for use thereof.

The instructional material included in the kit comprises instructions for preventing or treating a disease or disorder contemplated within the present disclosure. The instructional material recites the amount of, and frequency with which, at least one composition of the present disclosure should be administered to the mammal. In other embodiments, the kit further comprises at least one additional agent that prevents or treats the disease or disorder contemplated within the present disclosure.

Those skilled in the art will recognize, or be able to ascertain using no more than routine experimentation, numerous equivalents to the specific procedures, embodiments, claims, and examples described herein. Such equivalents are considered to be within the scope of this present disclosure and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application.

### EXAMPLES

These examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1. Determine Whether PDE9 Inhibitor PF-4447943 Blocks Psilocybin Induced Head Twitch Behavior

Illustrated herein is a dose-dependent decrease in hallucinogenic response (head-twitch response, HTR) to psilocybin administration by adding a PDE9 inhibitor.

In this experiment, it was determined whether the number of head twitches in 10 minutes in response to psilocybin administration in 7-10 week old C57B6/J mice (equal numbers of male and female) were reduced by PF-4447943, a prototypical PDE9 inhibitor. The experiment began with the mice receiving a 20-minute pretreatment with PF-4447943 or its associated vehicle (5% Cremophor, 5% dimethyl sulfoxide (DMSO), and 90% saline) followed by the test dose of psilocybin. Head twitch was assessed for 10 minutes in an automated system using implanted magnets in the mouse's ears. At least one week elapsed prior to reuse, and mice were used maximally twice. As shown in FIG. 1, HTR was reduced with the administration of psilocybin following pretreatment with PF-4447943 compared to the administration of psilocybin following pretreatment with the PF-4447943 vehicle. For instance, average HTR over 10 min in mice receiving 3 mg/kg psilocybin and no PF-4447943 was about 30, while average HTR over 10 min in mice receiving 3 mg/kg psilocybin plus 3 mg/kg PF-4447943 was about 20.

In another experiment, a psilocybin dose-response curve was generated in the presence and absence of a fixed dose of pre-administered PF-4447943. FIG. 2 depicts the results of HTR in mice receiving administration of a fixed dose of PF-4447943 (3 mg/kg IP) and various doses of psilocybin (0, 0.3, 1, 3, 10 mg/kg IP), in comparison with administration of psilocybin plus the PF-4447943 vehicle alone as a control. As shown in FIG. 2, pretreatment with 3 mg/kg PF-4447943 reduced HTR by about 40% across all doses of psilocybin.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method of treating or preventing a neuropsychiatric disorder in a subject comprising: administering to the subject a therapeutically effective amount of a serotonergic psychedelic drug and a therapeutically effective amount of a PDE9 inhibitor, wherein the administration treats or prevents the neuropsychiatric disorder.
Embodiment 2. The method of embodiment 1, wherein the PDE9 inhibitor prolongs at least one biological activity of the serotonergic psychedelic drug in the subject.
Embodiment 3. The method of embodiment 1, wherein the PDE9 inhibitor mitigates at least one undesirable biological activity of the serotonergic psychedelic drug in the subject.
Embodiment 4. The method of embodiment 1, wherein the PDE9 inhibitor reduces the abuse potential of the serotonergic psychedelic drug in the subject.
Embodiment 5. The method of any one of embodiments 1-4, wherein the neuropsychiatric disorder is selected from the group consisting of a major depressive disorder (MDD, including major depressive episode), a major depressive episode in bipolar disorder (bipolar depression), depressive episodes associated with bipolar I or II disorder (bipolar depression), a persistent depressive disorder (dysthymia), a disruptive mood dysregulation disorder, a premenstrual dysphoric disorder, a substance/medication-induced depressive disorder, a depressive disorder due to another medical condition, other specified depressive disorder, unspecified depressive disorder, an anxiety disorder, an obsessive-compulsive disorder, a posttraumatic stress disorder, an addictive disorder, treatment resistant depression (TRD), a generalized anxiety disorder (GAD), a post-traumatic stress disorder (PTSD), adjunctive treatment of major depression, eating disorders including anorexia and bulimia, depression associated with neurodegenerative disorders such as Parkinson's Disease, Alzheimer's Disease, dementias, and ALS, traumatic brain injury, suicidal thoughts and behaviors, chronic pain, a persistent depressive disorder, seasonal affective disorder (SAD), psychotic depression, peripartum (postpartum) depression, a premenstrual dysphoric disorder (PMDD), situational depression, and any combinations thereof.
Embodiment 6. The method of any one of embodiments 1-5, wherein the serotonergic psychedelic drug and the PDE9 inhibitor are co-administered to the subject.
Embodiment 7. The method of any one of embodiments 1-5, wherein the PDE9 inhibitor is administered to the subject before the serotonergic psychedelic drug is administered to the subject.
Embodiment 8. The method of any one of embodiments 1-5, wherein the serotonergic psychedelic drug is administered to the subject before the PDE9 inhibitor is administered to the subject.
Embodiment 9. The method of any one of embodiments 1-5, wherein the serotonergic psychedelic drug and the PDE9 inhibitor are co-formulated as a pharmaceutical composition.
Embodiment 10. The method of any one of embodiments 1-5, wherein the serotonergic psychedelic drug and the PDE9 inhibitor are independently administered to the subject by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, intravenous and any combinations thereof.
Embodiment 11. The method of any one of embodiments 1-10, wherein the serotonergic psychedelic drug is selected from the group consisting of Psilocin, Psilocybin, Bufotenin, Baeocystin, Aeruginascin, 5-MeO-DMT, N,N-Dimethyltryptamine (DMT), 5-Bromo-DMT, N-Methyl-N-ethyltryptamine (MET), N-Methyl-N-isopropyltryptamine (MiPT), N-Methyl-N-propyltryptamine (MPT), N,N-Diethyltryptamine (DET), N-Ethyl-N-isopropyltryptamine (EiPT), N-Methyl-N-butyltryptamine (MBT), N-Propyl-N-isopropyltryptamine (PiPT), N,N-Dipropyltryptamine (DPT), N,N-Diisopropyltryptamine (DiPT), N,N-Diallyltryptamine (DALT), N,N-Dibutyltryptamine (DBT), N-Ethyltryptamine (NET), N-Methyltryptamine (NMT), Trimethyltryptamine (TMT), α-Methyltryptamine, α-Ethyltryptamine, α,N-DMT, α,N ,N-Trimethyltryptamine, Ethocybin, 4-HO-MET, 4-HO-DET, 4-HO-MPT, 4-HO-MiPT, 4-HO-MALT, 4-HO-DPT, 4-HO-DiPT, 4-HO-DALT, 4-HO-DBT, 4-HO-DSBT, 4-HO-uMT, 4-HO-MPMI, 4-HO-TMT, 4-HO-1,N,N-TMT, 4-HO-5-MeO-DMT, 4-AcO-DMT, 4-AcO-MET, 4-AcO-MALT, 4-AcO-DET, 4-AcO-EiPT, 4-AcO-DPT, 4-AcO-DiPT, 4-AcO-DALT, 4-MeO-DMT, 4-MeO-MiPT, 5-MeO—N MT, 5-MeO-MET, 5-MeO-MPT, 5-MeO-MiPT, 5-MeO-DET, 5-MeO-Ei PT, 5-MeO-EPT, 5-MeO-Pi PT, 5-MeO-DPT, 5-MeO-DiPT, 5-MeO-DALT, 5-MeO-uMT, 5-MeO-2,N,N-TMT, 5-MeO-7,N,N-TMT, 5-MeO-α,N-DMT, 4-F-5-MeO-DMT, 5-Me-MiPT, 5-HO-DiPT, 2-α-DMT, 4-Me-uMT, 4-Me-αET, 7-Me-αET, 4,5-DHP-AMT, 4,5-DH P-DMT, 4,5-MDO-DMT, 4,5-MDO-DiPT, 5,6-MDO-DiPT, 5,6-MDO-MiPT, 5-Fluoro-uMT, 6-Fluoro-αMT, 6-Fluoro-DMT, N,N-Tetramethyl enetryptamine (Pyr-T), 4-HO-pyr-T, 5-MeO-pyr-T, RU-28306 (4,a-Methylene-N,N-DMT), O-4310 (6-Fluoro-1-Isopropyl-4-HO-DMT), CP-132,484 (4,5-DHP-1-Methyltryptamine), Dimemebfe (5-MeO-BFE), 5-MeO-DiBF, Ibogaine, Voacangine, Lysergic acid diethylamide (LSD), Lysergic acid amide (LSA), Lysergic acid diamide, N1-Methyl-lysergic acid diethylamide, 1-Propionyl-lysergic acid diethylamide, 1-cyclopropanoyl-d-lysergic acid diethylamide, 1-valeryl-D-lysergic acid diethylamide, 6-Allyl-6-nor-lysergic acid diethylamide, 6-Butyl-6-nor-lysergic acid diethylamide, 6-Ethyl-6-nor-lysergic acid diethylamide, 1-Propionyl-6-Ethyl-6-nor-lysergic acid diethylamide, 6-Propyl-6-nor-lysergic acid diethylamide, 6-Cyclopropyl-6-nor-lysergic acid diethylamide, 6-nor-Lysergic acid diethylamide, Lysergic acid ethylamide, Lysergic acid α-hydroxyethylamide, Lysergic acid 2-butyl amide, Lysergic acid 3-pentyl amide, Lysergic acid methyl ester, Lysergic acid 2,4-dimethylazetidide, Lysergic acid piperidine, N,N-Dimethyl-lysergamide, Methylisopropyllysergamide, N,N-Diallyllysergamide, N-Pyrrolidyllysergamide, N-Morpholinyllysergamide, 1-methyl-lysergic acid butanolamide, Lysergic acid P-propanolamide, Lysergic acid 1-butanolamide, Mescaline, Lophophine, Isomescaline, Cyclopropylmescaline, Thioisomescaline (2-TIM, 3-TIM, and 4-TIM), 4-Desoxymescaline, Jimscaline, Escaline, Metaescaline, Thiometaescaline (3-TME, 4-TME, and 5-TME), Trisescaline, Thiotrisescaline (3-T-TRIS and 4-T-TRIS), Symbescaline, Asymbescaline, Thiosymbescaline (3-TSB and 4-TSB), Phenescaline, Allylescaline, Methallylescaline, Proscaline, Isoproscaline, Metaproscaline, Thioproscaline, Buscaline, Thiobuscaline, α-ethylmescaline, Ariadne, Macromerine, MEPEA, TOM (2-TOM and 5-TOM), Bis-TOM, TOMSO (2-methoxy-4-methyl-5-methylsulfinylamphetamine), TOET (2-TOET and 5-TOET), BOH, BOM (13-Methoxy-mescaline), beta-D, 4-D, DME, F-2, F-22, FLEA, MDPH, MDMP, Propynyl, 2C family (2,5-dimethoxy, 4-substituted phenethylamines), βk-2C-B, 2C—B, 2CB-2EtO, 2CB-5EtO, 2CB-diEtO, 2C—B-FLY, 2C—B-BUTTERFLY, 2C—C, 2C-D, 2CD-2EtO, 2CD-diEtO, 2CD-5EtO, 2C-E, 2C-EF, 2C—F, 2C-G (2C-G-1, 2C-G-2, 2C-G-3, 2C-G-4, 2C-G-5, 2C-G-6, and 2C-G-N), 2C—H, 2C—I, 2CI-2EtO, 2C-iP, 2C—N, 2C—O, 2C—O-4, 2C—P, 2C-SE, 2C-T, 2CT-5EtO, 2C-T-2, 2CT-2-2EtO, 2CT-2-5EtO, 2CT-2-diEtO, 2C-T-4 (2C-T-4 and Ψ-2C-T-4), 2CT-4-2EtO, 2C-T-7, 2CT-7-2EtO, 2C-T-8, 2C-T-9, 2C-T-13, 2C-T-15, 2C-T-16, 2C-T-17, 2C-T-19, 2C-T-21, 2C-TFM, 2C—YN, BOB (13-Methoxy-2C-B), BOD (13-Methoxy-2C-D), BOHD (13-Hydroxy-2C-D), HOT-2, HOT-7, HOT-17, Indane derivatives comprising 2CB-Ind, Benzocyclobutene derivatives comprising 2C—BCB (TCB-2), NBOMe derivatives comprising NBOMe-mescaline, 2C—H—NBOMe, 2C—C—NBOMe, 2CBCB—NBOMe, 2CBFly-NBOMe, 2C—B—NBOMe, 2C—I—NBOMe, 2C-TFM-NBOMe, 2C-D-NBOMe, 2C-G-NBOMe, 2C-E-NBOMe, 2C—P—NBOMe, 2C-iP-NBOMe, 2C—CN—NBOMe, 2C—N—NBOMe, 2C-T-NBOMe, 2C-T-4-NBOMe, 2C-T-7-NBOMe, and DMBMPP, NBOH derivatives comprising 2C—C—NBOH, 2C—B—NBOH, 2C—I—NBOH, and 2C—CN—NBOH, NBMD derivatives comprising 2C—I-NBMD, NBF derivatives comprising 2C—C—NBF, 2C—B—NBF, and 2C—I—NBF, 3C family (3,5-dimethoxy, 4-substituted amphetamines) comprising 3C-E, 3C—P, 3C-DFE, and 3C—BZ, DOx family (2,5-dimethoxy, 4-substituted amphetamines) comprising DOAM, DOB, Meta-DOB, Methyl-DOB, DOBU, DOC, DOEF, DOET, DOI, DOM, Ψ-DOM, DON, DOPR, SOiPR, DOT, Meta-DOT, Ortho-DOT, and DOTFM, DMCPA, DMMDA, DMMDA-2, 2,5-dimethoxy-3,4-dimethylamphetamine, 4-methyl-2,5-dimethoxymethamphetamine, 2,N-dimethyl-4,5-methylenedioxy amphetamine, Dimethoxyamphetamine (2,4-DMA, 2,5-DMA, and 3,4-DMA), Trimethoxy amphetamine (TMA-2, TMA-6), Tetramethoxyamphetamine, Br-DragonFLY, TFMFly, 2-Bromo-4,5-methylenedioxyamphetamine, 4-Bromo-3,5-dimethoxyamphetamine, EEE, EEM, EME, EMM, EDMA, EIDA, Ethyl-J, Methyl-J, Ethyl-K, Mehtyl-K, IDNNA, Iris, MDAI, MDMAI, MDAT, MDMAT, MDAL, MDBU, MDBZ, MDDM, MDIP, MDMEOET, MDMEO, MDOH, MDHOET, MDPL, MDCPK, MDPR, MEDA, MEM, Mehtyl-DMA, MMDA, MMDA-2, 5-Mehtyl-MDA, MEE, MME, MPM, DiFMDA, 5-APB, 6-APB, 5-APDB, 6-APDB, 5-MAPB, 5-MAPDB, 6-MAPDB, 6-MAPB, 6-EAPB, 5-EAPB, Para-Methoxyamphetamine, Paramethoxymethamphetamine, 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Benzoxazine, Efavirenz, Substituted methylenedioxy-phenethylamines (MDxx) comprising 3,4-methylenedioxymethamphetamine (MDMA), MDA, 2,3-MDA, 5-Methyl-MDA, MMDA, MDEA, MBDB, MDAL, MDBU, MDBZ, MDDM, MDIP, MDMEOET, MDMEO, MDOH, MDHOET, MDPL, MDCPM, MDPR, BDB, MMDA-2, DiFMDA, EIDA, Ethyl-K, Lophophine, Substituted amphetamines, EDMA, Para-Methoxyamphetamine (PMA), Paramethoxymethamphetamine (PMMA), 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Substituted cathinones, Methylone, Ethylone, Eutylone, Butylone, Pentylone, 4-Ethyl methcathinone, 3-Methylmethcathinone, Substituted benzofurans, 5-AP B, 6-AP B, 5-APDB, 6-APDB, 5-MAPB, 5-MAPDB, 6-MAPDB, 6-MAPB, 5-EAPB, 6-EAPB, 5-MBPB, MDAT, MDMAT, 6-CAT, Tetralinylaminopropane, Trifluoromethylaminoindane, Ethyltrifluoromethylaminoindane, 5-Iodo-2-aminoindane, MMAI, MDAI, MDMAI, Indanylaminopropane, Naphthylaminopropane, 4-chlorophenylisobutylamine, 4-Methylphenylisobutylamine, Ariadne, α-methyltryptamine, 5-MeO-uMT, α-ethyltryptamine, 4-Me-αET, 7-Me-αET, 5-MeO-αET, 5-MeO-MiPT, Δ⁹-THC, CBD, CBN, THCV, (C6)-CP 47,497, (C9)-CP 47,497, 1-Butyl-3-(2-methoxybenzoyl)indole, 1-Butyl-3-(4-methoxybenzoyl)indole, 1-Pentyl-3-(2-methoxybenzoyl)indole, 2-Isopropyl-5-methyl-1-(2,6-di hydroxy-4-nonylphenyl)cyclohex-1-ene, 4-HTMPIPO, 4-Nonylphenylboronic acid, 5Br—U R-144, 5Cl-APINACA, 5Cl—U R-144, 5F-3-pyridinoylindole, 5F-AB-FUPPYCA, 5F-ADB-PINACA, 5F-ADBICA, 5F-ADB, 5F-AMB, 5F-APINACA, 5F-CUMYL-PINACA, 5F-EMB-PINACA, 5F-NNE1, 5F—PB-22, 5F—PCN, 5F—PY-PICA, 5F—PY—P INACA, 5F-SDB-006, HHC, A-796,260, A-834,735, A-836,339, A-955,840, A-40174, A-41988, A-42574, AB-001, AB-CH FU PYCA, AB-CHMFUPPYCA, AB-CHMINACA, AB-FUBICA, AB-FUBINACA 2-fluorobenzyl isomer, AB-FUBINACA, AB-PICA, AB-PINACA, Abnormal cannabidiol, ADAMANTYL-THPINACA, ADB-CHMINACA, ADB-FUBICA, ADB-FUBINACA, ADB-PINACA, ADBICA, ADS B—FU B-187, Ajulemic acid, AM-087, AM-411, AM-630, AM-679, AM-694, AM-855, AM-883, AM-905, AM-906, AM-919, AM-926, AM-938, AM-1220, AM-1221, AM-1235, AM-1241, AM-1248, AM-1346, AM-1387, AM-1714, AM-2201, AM-2232, AM-2233, AM-2389, AM-4030, AM-4113, AM-6527, AM-6545, AM-251, AM-281, AM-404, AMB-CHMINACA, AMB-FUBINACA, AMG-1, AMG-3, AMG-36, AMG-41, APICA, APINACA, APP-FUBINACA, Arachidonoyl SEROTONIN, ACEA, ACPA, Arvanil, AZ-11713908, BAY 38-7271, BAY 59-3074, BIM-018, Biochanin A, BML-190, Nabidrox (Canbisol), Cannabicyclohexanol, Cannabipiperidiethanone, CAY-10401, CAY-10429, CAY-10508, CB-13, CB-25, CB-52, CB-86, CB-86, CBS-0550, CP 47,497, CP 55,244, CP 55,940, CUMYL-5F-PICA, CUMYL-BICA, CUMYL-PICA, CUMYL-PINACA, CUMYL-THPINACA, Dexanabinol, Dimethylheptylpyran, Drinabant, Dronabinol, EAM-2201, EMB-FUBINACA, FAB-144, FDU-NNE1, FDU-PB-22, FUB-144, FUB-APINACA, FUB-JWH-018, FUB—PB-22, FUBIMINA, Genistein, GW-405,833, GW-842,166X, Hemopressin, HU-210, HU-243, HU-308, HU-320, HU-331, HU-336, HU-345, HU-910, Ibipinabant, IDFP, JNJ 1661010, JNJ 1661010, JTE-907, JTE 7-31, JWH-007, JWH-015, JWH-018, JWH-019, JWH-030, JWH-051, JWH-073, JWH-081, JWH-098, JWH-116, JWH-122, JWH-133, JWH-139, JWH-147, JWH-149, JWH-161, JWH-164, JWH-167, JWH-175, JWH-176, JWH-182, JWH-184, JWH-185, JWH-192, JWH-193, JWH-194, JWH-195, JWH-196, JWH-197, JWH-198, JWH-199, JWH-200, JWH-203, JWH-210, JWH-229, JWH-249, JWH-250, JWH-251, JWH-302, JWH-307, JWH-359, JWH-369, JWH-370, JWH-398, JWH-424, JZL184, JZL195, Kaempferol, KM-233, L-759,633, L-759,656, LASSBio-881, LBP-1, Leelamine, Levonantradol, LH-21, LY-320,135, LY-2183240, NAM-2201, MDM-2201, MDA-7, MDA-19, MDA-77, MDMB-CHMICA, MDMB-CHMINACA, MDMB-FUBINACA, Menabitan, MEPIRAPIM, Methanandamide, MJ-15, MK-9470, MMB-2201, MN-18, MN-25, Nabazenil, Nabilone, Nabitan, Naboctate, NESS-0327, NESS-040C5, NIDA-41020, NM-2201, NMP-7, NNE1, Nonabine O-224, O-581, O-585, O-606, O-689, O-774, O-806, O-823, O-889, O-1057, O-1125, O-1184, O-1191, O-1238, O-1248, O-1269, O-1270, O-1376, O-1399, O-1422, O-1601, O-1602, O-1624, O-1656, O-1657, O-1660, O-1812, O-1860, O-1861, O-1871, O-1918, O-2048, O-2050, O-2093, O-2113, O-2220, O-2365, O-2372, O-2373, O-2383, O-2426, O-2484, O-2545, O-2654, O-2694, O-2715, O-2716, O-3223, O-3226, Oleoylethanolamide, Olvanil, Org 27569, Org 27759, Org 2831, Org 28611, Org 29647, Otenabant, Palmitoylethanolamide, Parahexyl, PF-03550096, PF-04457845, PF-622, PF-750, PF-3845, PF-514273, PHOP, PipISB, Pimabine, Pravadoline, Pregnenolone, PSB—SB-487, PSB—SB-1202, PTI-1, PTI-2, PX-1, PX-2, PX-3, QUCHIC, QUPIC, RCS-4, RCS-8, Rimonabant, Rosonabant, RTI-371, S-444,823, SDB-006, SER-601, SPA-229, SR-144,528, STS-135, Surinabant, Taranabant, Tedalinab, THC-O-acetate, THC-O-phosphate, THJ-018, THJ-2201, Tinabinol, TM-38837, UR-144, URB-447, URB-447, URB-597, URB-602, URB-754, VCHSR, VDM-11, VSN-16, WIN 54,461, WIN 55,212-2, WIN 56,098, XLR-11, Yangonin, Harmaline, harmala alkaloids, other beta-carbolines, active constituents of ayahuasca, Salvinorin A, Salvinorin B methoxymethyl ether, Salvinorin B ethoxymethyl ether, Piperazines, pFPP, TFMPP, Myristicin, Elemicin, Cryogenine (Vertine), Atropine, Scopolamine, Hyoscyamine, Ibotenic acid, Muscimol, TiHKAL, 2-Me-DET, 4-HO-DBT, 4-HO-DET, 4-HO-DiPT, 4-HO-EPT, 4-HO-McPT, 4-HO-MET, 4-HO-MiPT, 4-HO-MPMI, 4-HO-MPT, 4-HO-uMT, 4-Me-uMT, 4-MeO-MiPT, 4-PrO-DMT, 4,5-MDO-DiPT, 4,5-MDO-DMT, 5-Ethoxy-uMT, 5-Fluoro-AET, 5-Fluoro-DET, 5-Fluoro-DMT, 5-Fluoro-EPT, 5-Fluoro-MET, 5-MeO-AET, 5-MeO-αMT, 5-MeO-DALT, 5-MeO-DET, 5-MeO-DPT, 5-MeO-EiPT, 5-MeO-MALT, 5-MeO-MiPT, 5-MeO-MPMI, 5-MeO-pyr-T, 5-MeS-DMT, 5-MeO-2-TMT, 5-TFM-DMT, 5-TFMO-DMT, 5,6-MDO-DiPT, 5,6-MDO-DMT, 5,6-MDO-MiPT, 5,6-MeO-MiPT, 5,N,N-TMT, 5F-MPMI, 6-Fluoro-DET, 6-Fluoro-DMT, 6-MeO-THH, 7-Chloro-AMT, 7-F-5-MeO-MET, 4-Acetoxy-DET, 4-Acetoxy-DiPT, 4-Acetoxy-MET, 4-Acetoxy-MiPT, O-Acetylbufotenine, O-Acetylpsilocin, Aeruginascin, Alpha,N-DMT, Alpha,N,O-TMS, Baeocystin, 5-Bromo-DMT, Bufotenin, 5-Chloro-uMT, DALT, Dibutyltryptamine, Diethyltryptamine, Diisopropyltryptamine, 5,N-Dimethyl-N-isopropyltryptamine, Dimethyltryptamine, N,N-Dimethyltryptamine, Dipropyltryptamine, 2,α-Dimethyltryptamine, Ethocybin, Ethylisopropyltryptamine, A-Ethyltryptamine, 5-Fluoro-AMT, 4-Fluoro-5-methoxy-DMT, FT-104, 5-MeO-DMT, 5-Methoxy-N,N-diisopropyltryptamine, 4-Methyl-α-ethyltryptamine, N-Methyl-N-ethyltryptamine, Methylbutyltryptamine, Methylisopropyltryptamine, Alpha-Methylserotonin, Alpha-Methyltryptamine, N-Methyltryptamine, MPMI, Norbaeocystin, Propylisopropyltryptamine, 2,N,N-TMT, A,N,N-Trimethyltryptamine, Ketamine, Esketamine, Arketamine, Mitragynine, Yohimbine, and any combinations thereof.
Embodiment 12. The method of any one of embodiments 1-10, wherein the serotonergic psychedelic drug is a tryptamine or an ergoline.
Embodiment 13. The method of any one of embodiments 1-12, wherein the PDE9 inhibitor is selected from the group consisting of aminophylline, pentoxifylline, theobromine, paraxanthine, PF-04447913, PF-04447943, PF-04447953, BAY 73-6691, E 2027, CRD 773, BI 409306, CRD 740, TT 00920, BAY 7081, FRM 16606, HUC1-288, WYQ-C36D, and any combinations thereof.
Embodiment 14. The method of any one of embodiments 1-12, wherein the PDE9 inhibitor is selected from the group consisting of 1-{[2-(3-isopropyl-7-oxo-6,7-dihydro-1H-pyrazolo[4,3-d]pyrimidin-5-ylmethyl)-phenoxy]-acetyl}-pyrrolidine-2-carbo-xylic acid; 1-{[2-(1-cyclopentyl-4-oxo-4,5-dihydro-1H-pyrazolo[3,4-d]pyrim-idin-6-ylmethyl)-phenoxy]-acetyl}-pynOlidine-2(S)-carboxylic acid 3-isopropyl-5-[2-(2-oxo-2-piperazin-1-yl-ethoxy)-benzyl]-1,6-dihydro-pyrazolo[4,3-d]pyrimidin-7-one; 1-cyclopentyl-6-[2-(2-oxo-2-piperazin-1-yl-eth-oxy)-benzyl]-1,5-dihydro-pyrazolo[3,4-d]pyrimidin-4-one 3-isopropyl-5-[2-(2-morpholin-4-yl-2-oxo-ethoxy)-benzyl]-1,6-dihydro-pyra-zolo[4,3-d]pyrimidin-7-one; 3-isopropyl-5-[2-(2-oxo-2-pyrrolidin-1-yl-etho-xy)-benzyl]-1,6-dihydro-pyrazolo[4,3-d]pyrimidin-7-one; 5-{2-[2-(4-ethyl-piperazin-1-yl)-2-oxo-ethoxy]-benzyl}-3-isopropyl-1,6-di-hydro-pyrazolo[4,3-d]pyrimidin-7-one; N,N-diethyl-2-[2-(3-isopropyl-7-oxo-6,7-dihydro-1H-pyrazolo[4,3-d]pyrimidin-5-ylmethyl)-phenoxy]-acetamide; 1-{[2-(3-isopropyl-7-oxo-6,7-dihydro-1H-pyrazolo[4,3-d]pyrimidin-5-ylmeth-yl)-phenoxy]-acetyl}-pyrrolidine-2-carboxy lie acid methyl ester; 4-{[2-(3-isopropyl-7-oxo-6,7-dihydro-1H-pyrazolo[4,3-d]pyrimidin-5-ylmeth-yl)-phenoxy]-acetyl}-piperazine-1-carboxylic acid tert-butyl ester; N-(2-dimethylamino-ethyl)-2-[2-(3-isopropyl-7-oxo-6,7-dihydro-1H-pyrazolo-[4, 3-d]pyrimidin-5-ylmethyl)-phenoxy]-acetamide; 1-{[2-(1-cyclopentyl-4-ox-o-4,5-dihydro-1H-pyrazolo[3,4-d]pyrimidin-6-ylmethyl)-phenoxy]-acetyl}-pyr-rolidine-2-carboxylic acid methyl ester; 4-{[2-(1-cyclopentyl-4-oxo-4,5-di-hydro-1H-pyrazolo[3,4-d]pyrimidin-6-ylmethyl)-phenoxy]-acetyl}-piperazine-1-carboxylic acid tert-butyl ester; l-cyclopentyl-6-[2-(2-oxo-2-pyrrolidin-1-yl-ethoxy)-benzyl]-1,5-dihydro-pyrazolo[3,4-d]pyrimidin-4-one; 1-cyclopentyl-6-[2-(2-morpholin-4-yl-2-oxo-ethoxy)-benzyl]-1,5-dihydro-pyrazolo[3,4-d]pyrimidin-4-one; 2-[2-(1-cyclopentyl-4-oxo-4,5-dihydro-1H-pyr-azolo[3,4-d]pyrimidin-6-ylmethyl)-phenoxy]-N-(2-dimethylamino-ethyl)-aceta-mide; l-cyclopentyl-6-{2-[2-(4-ethyl-piperazin-1-yl)-2-oxo-ethoxy]-benzyl}-1,5-dihydro-pyrazolo[3,4-d]pyrimidin-4-one; 2-[2-(1-cyclopentyl-4-oxo-4,5-dihydro-1H-pyrazolo[3,4-d]pyrimidin-6-ylmethyl)-phenoxy]-N,N-diethyl-acet-amide; [2-(3-isopropyl-7-oxo-6, 7-dihydro-1H-pyrazolo[4,3-d]pyrimidin-5-ylm-ethyl)-phenoxy]-acetic acid; [2-(1-cyclopentyl-4-oxo-4,5-dihydro-1H-pyrazo-lo[3,4-d]pyrimidin-6-ylmethyl)-phenoxy]-acetic acid; 3-isopropyl-5-[2-(5-chloro-2-morpholin-4-yl-ethoxy)-benzyl]-1,6-dihydro-p-yrazolo[4,3-d]pyrimidin-7-one; 3-isopropyl-5-[2-(2-pyrrolidin-1-yl-ethoxy)-benzyl]-1,6-dihydro-pyrazolo[4,3-d]pyrimidin-7-one; 3-isopropyl-5-[2-(2-morpholin-4-yl-ethoxy)-cyclohexylmethyl]-1, 6-dihydro-pyrazolo[4,3-d]pyrimidin-7-one; 5-[5-fluoro-2-(2-morpholin-4-yl-ethoxy)-be-nzyl]-3-isopropyl-1,6-dihydro-pyrazolo[4,3-d]pyrimidin-7-one; 3-cyclopentyl-5-[5-fluoro-2-(2-morpholin-4-yl-ethoxy)-benzyl]-1, 6-dihydro-1-pyrazolo[4,3-d]pyrimidin-7-one; 3-isopropyl-5-[2-(2-morpholin-4-yl-ethoxy-)-benzyl]-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 2-[2-(2-morpholin-4-yl-ethoxy)-benzyl]-9-(tetrahydro-furan-3-yl)-1,9-dihydro-purin-6-one; 5-[2-(2-diethylamino-ethoxy)-benzyl]-3-isop-ropyl-1,6-dihydro-pyrazolo[4,3-d]pyrimidin-7-one; 3-cyclopentyl-5-[2-(2-mo-rpholin-4-yl-ethoxy)-benzyl]-1,6-dihydro-pyrazolo[4,3-d]pyrimidin-7-one; 3-cyclobutyl-5-[2-(2-morpholin-4-yl-ethoxy)-benzyl]-1,6-dihydro-pyrazolo[-4,3-d]pyrimidin-7-one; 9-(1(R),2-dimethyl propyl)-[2-(2-morpholin-4-yl-eth-oxy)-benzyl]-1,9-dihydro-purin-6-one; 9-(2-methyl-butyl)-2-[2-(2-morpholin-4-yl-ethoxy)-benzyl]-1,9-dihydro-purin-6-one; 9-cyclopentyl-2-[2-(2-morph-olin-4-yl-ethoxy)-benzyl]-1,9-dihydro-purin-6-one; 5-[2-(2-morpholin-4-yl-ethoxy)-benzyl]-3-pyridin-3-yl-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 9-isopropyl-2-[2-(2-morpholin-4-yl-ethoxy)-benzyl]-1,9-dihy-dro-purin-6-one; 2-[2-(2-morpholin-4-yl-ethoxy)-benzyl]-9-(tetrahy dro-fura-n-2-ylmethyl)-1,9-dihydro-purin-6-one; 9-(1-isopropyl-2-methyl-propyl)-2-[2-(2-morpholin-4-yl-ethoxy)-benzyl]-1,9-dihydro-purin-6-one; 9-(1-ethyl-propyl)-2-[2-(2-morphobn-4-yl-ethoxy)-benzyl]-1, 9-dihydro-pur-in-6-one; 9-cyclopentyl-8-methyl-2-[2-(2-morphobn-4-yl-ethoxy)-benzyl]-1,-9-dihydro-purin-6-one; 3-cyclopentyl-5-[2-(2-morphobn-4-yl-ethoxy)-benzyl-]-3,6-dihydro-[1,2,3]triazolo[4,5-d]pyrimidin-7-one; 1-cyclopentyl-6-[2-(2-morpholin-4-yl-ethoxy)-benzyl]-1,5-dihydro-pyrazolo-[3,4-d]pyrimidin-4-one; 9-cyclopentyl-2-[2-(3-morpholin-4-yl-propoxy)-benz-yl]-1,9-dihydro-purin-6-one; N-[(1R,2S)2-(3-isopropyl-7-oxo-6,7-dihydro-1H-pyrazolo[4,3-d]pyrimidin-5-ylmethyl)-cyclohex-1-yl]-2-pyrrobdin-1-yl-acetamide; N-[(1R,2S)2-(3-isopropyl-7-oxo-6,7-dihydro-1H-pyrazolo[4,3-d]pyrim-idin-5-ylmethyl)-cyclohex-1-yl]-2-morphobn-4-yl-acetamide; 2-diethylamino-N-[(1R,2S)2-(3-isopropyl-7-oxo-6,7-dihydro-1H-pyrazolo[4,3-d]pyrimidin-5-ylmethyl)-cyclohex-1-yl]-acetamide; l-{[(1R,2S)2-(3-isoprop-yl-7-oxo-6,7-dihydro-1H-pyrazolo[4,3-d]pyrimidin-5-ylmethyl)-cyclohex-1-yl-carbamoyl]-methyl}-pyrrobdine-2(S)-carboxylic acid methyl ester; 2-cyclobutylamino-N-[(1R,2S)2-(3-isopropyl-7-oxo-6,7-dihydro-1H-pyrazolo[-4,3-d]pyrimidin-5-ylmethyl)-cyclohex-1-yl]-acetamide; or 2-cyclopropylamino-N-[(1R,2S)2-(3-isopropyl-7-oxo-6,7-dihydro-1H-pyrazolo-[4,3-d]pyrimidin-5-ylmethyl)-cyclohex-1-yl]-acetamide; IMR-687, (a potent inhibitor of PDE9A), BAY73-6691, and PF-04447943, PF—4181366, and stereoisomers, pharmaceutically acceptable salts, solvates and prodrugs thereof, and any combinations thereof.
Embodiment 15. The method of any one of embodiments 1-12, wherein the PDE9 inhibitor is selected from the group consisting of:
a salt of (S)-7-(2-methoxy-3,5-dimethylpyridin-4-yl)-1-(tetrahydrofuran-3-yl)-1H-pyrazolo[4,3-c]quinolin-4(5H)-one and an acid selected from the group consisting of hydrochloric acid, hydrobromic acid, sulfuric acid, nitric acid, phosphoric acid, malonic acid, maleic acid, tartaric acid, methanesulfonic acid, benzenesulfonic acid and toluenesulfonic acid;
(S)-7-(2-methoxy-3,5-dimethylpyridin-4-yl)-1-(tetrahydrofuran-3-yl)-1H-pyrazolo[4,3-c]quinolin-4(5H)-one monomaleate salt;
(S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt;
a crystal of the salt;
a crystal of (S)-7-(2-methoxy-3,5-dimethylpyridin-4-yl)-1-(tetrahydrofuran-3-yl)-1H-pyrazolo[4,3-c]quinolin-4(5H)-one monomaleate salt, having a diffraction peak at a diffraction angle (2θ±0.2°) of 10.1° in powder X-ray diffraction; and
a crystal of (S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt, having a diffraction peak at a diffraction angle (2θ±0.2°) of 9.9° in powder X-ray diffraction.
   Embodiment 16. The method of any one of embodiments 1-12, wherein the PDE9 inhibitor is selected from the group consisting of:

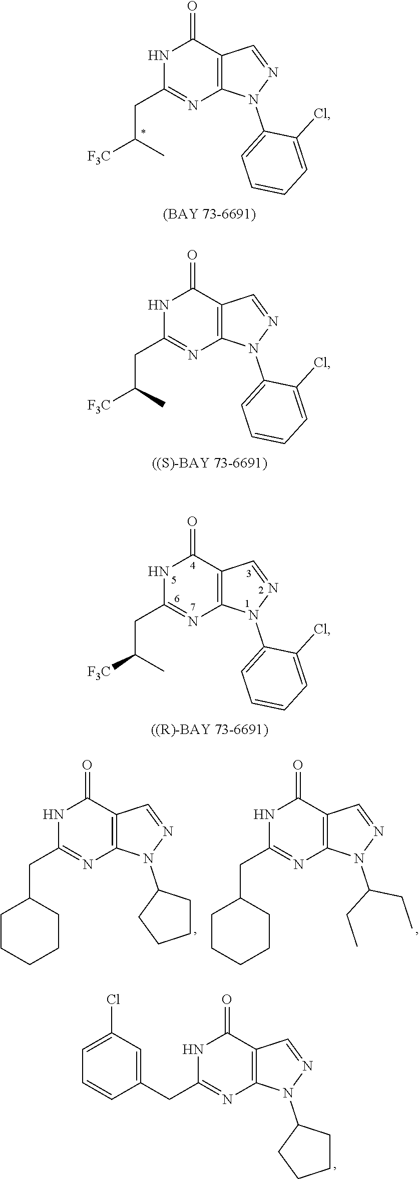

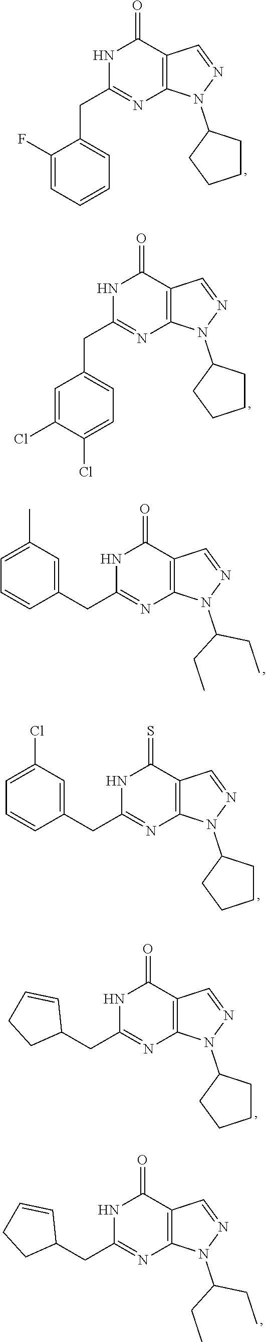 [SMILES: O=c1[nH]c(Cc2ccccc2F)nc2c1cnn2C1CCCC1 ; O=c1[nH]c(Cc2ccc(Cl)c(Cl)c2)nc2c1cnn2C1CCCC1 ; CCC(CC)n1ncc2c(=O)[nH]c(Cc3cccc(C)c3)nc21 ; S=c1[nH]c(Cc2cccc(Cl)c2)nc2c1cnn2C1CCCC1 ; O=c1[nH]c(CC2C=CCC2)nc2c1cnn2C1CCCC1 ; CCC(CC)n1ncc2c(=O)[nH]c(CC3C=CCC3)nc21]

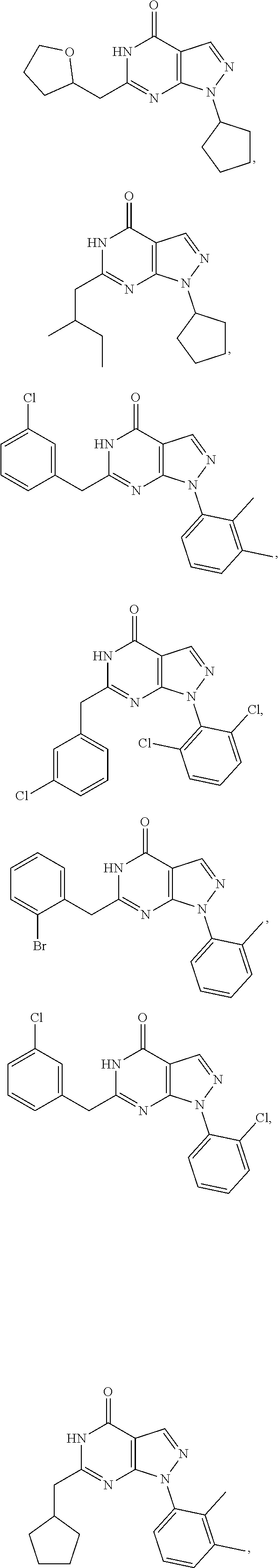

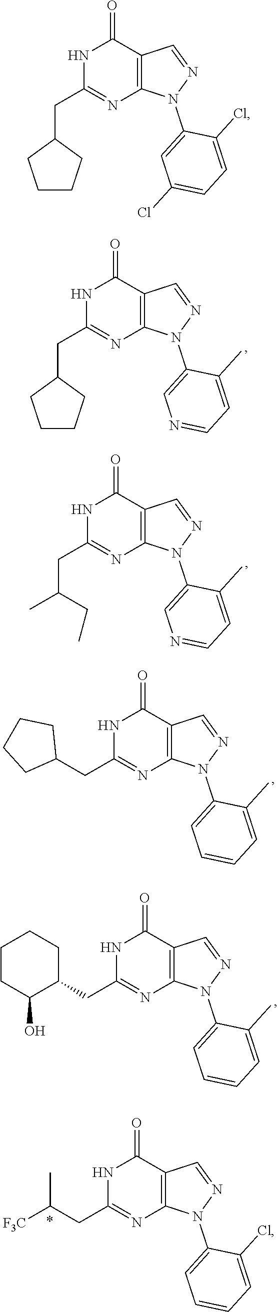

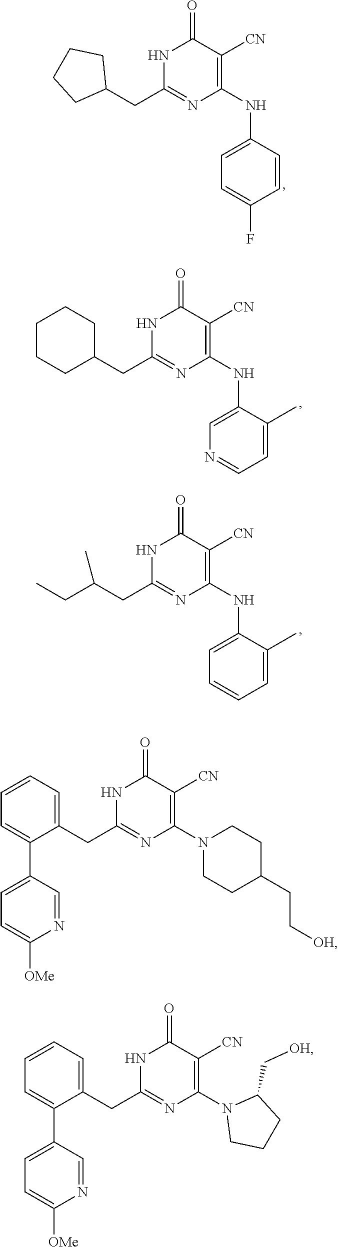

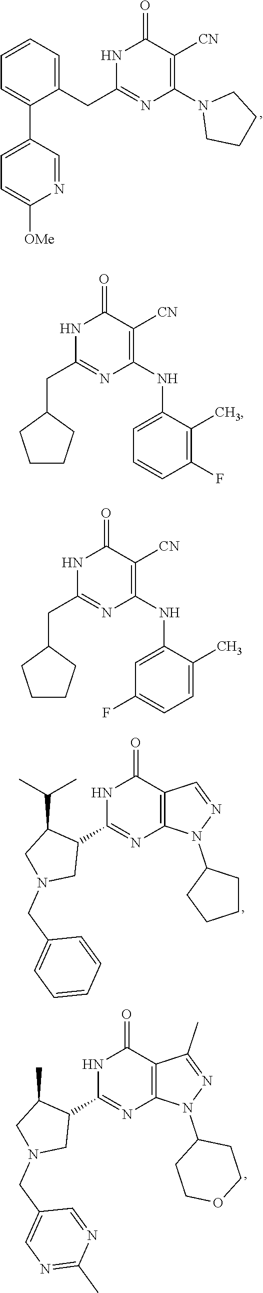

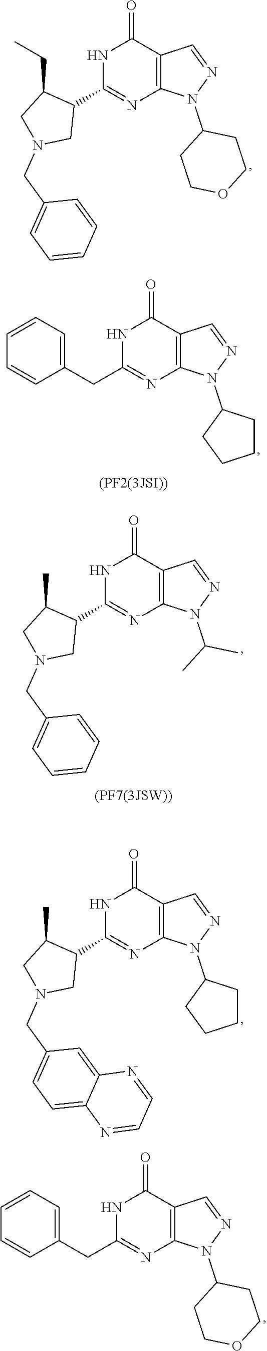

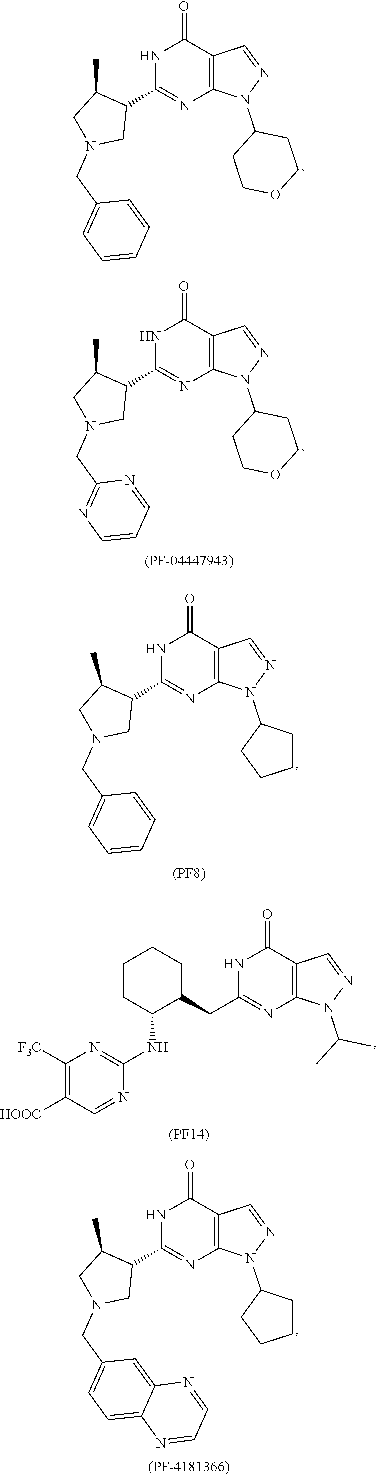

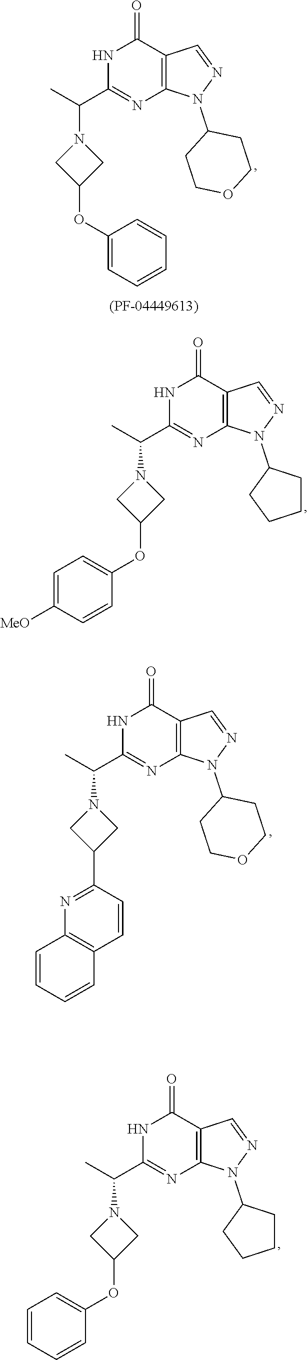 [SMILES: CC(c1nc2c(cnn2C2CCOCC2)c(=O)[nH]1)N1CC(Oc2ccccc2)C1 ; COc1ccc(OC2CN([C@H](C)c3nc4c(cnn4C4CCCC4)c(=O)[nH]3)C2)cc1 ; C[C@H](c1nc2c(cnn2C2CCOCC2)c(=O)[nH]1)N1CC(c2ccc3ccccc3n2)C1 ; C[C@H](c1nc2c(cnn2C2CCCC2)c(=O)[nH]1)N1CC(Oc2ccccc2)C1]

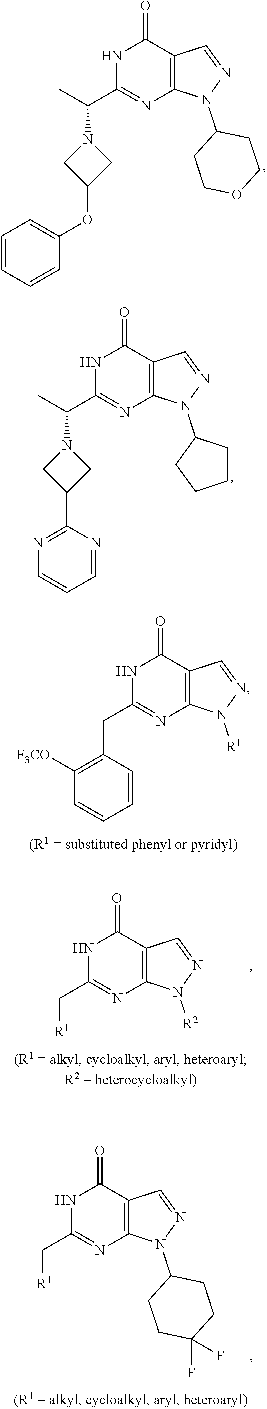

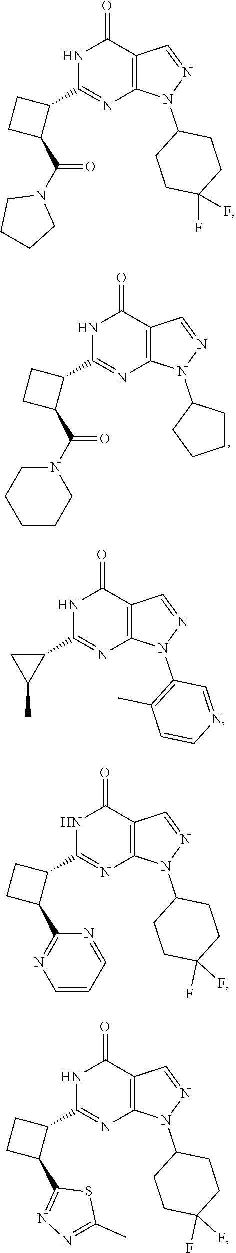

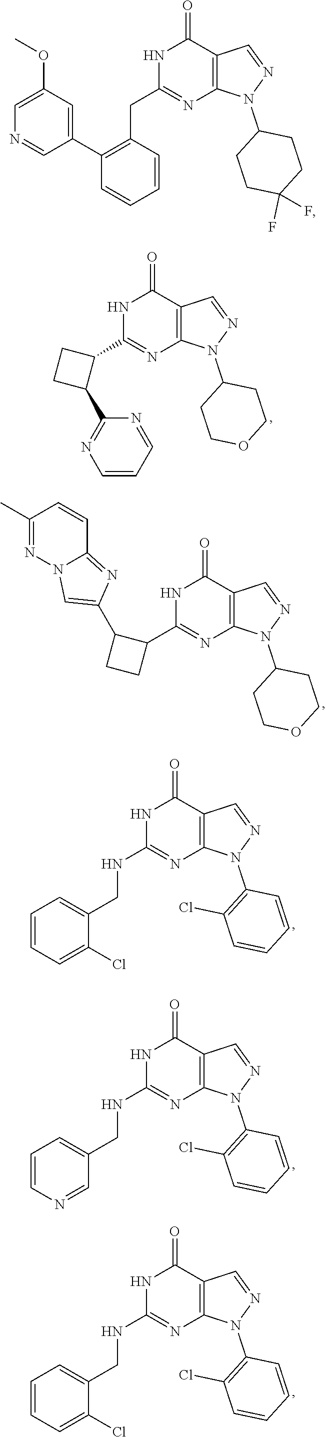

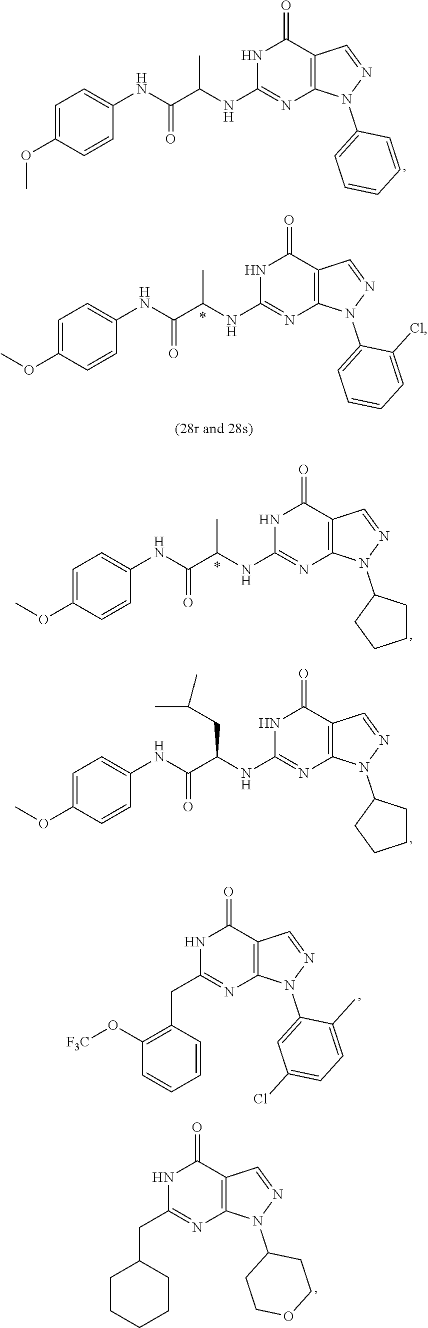

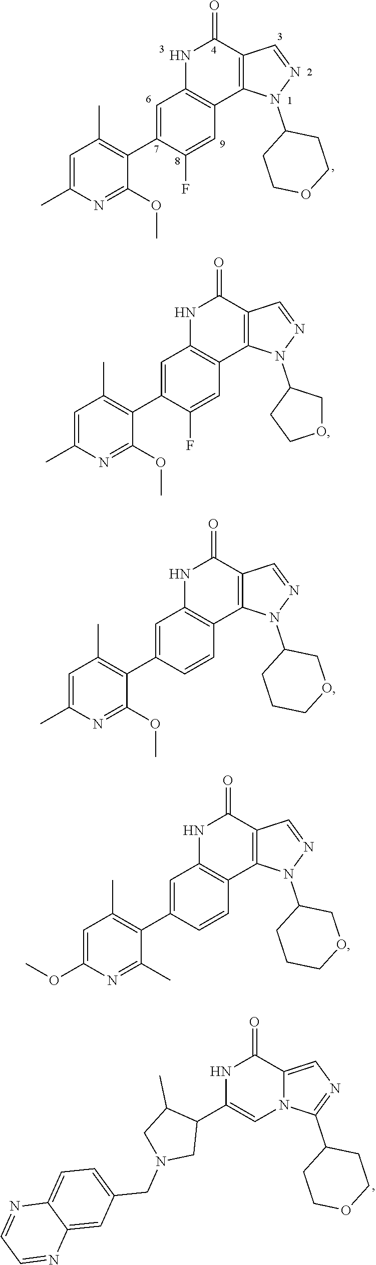 [SMILES: COc1nc(C)cc(C)c1-c1cc2[nH]c(=O)c3cnn(C4CCOCC4)c3c2cc1F ; COc1nc(C)cc(C)c1-c1cc2[nH]c(=O)c3cnn(C4CCOC4)c3c2cc1F ; COc1nc(C)cc(C)c1-c1ccc2c(c1)[nH]c(=O)c1cnn(C3CCCOC3)c12 ; COc1cc(C)c(-c2ccc3c(c2)[nH]c(=O)c2cnn(C4CCCOC4)c23)c(C)n1 ; CC1CN(Cc2ccc3nccnc3c2)CC1c1cn2c(C3CCOCC3)ncc2c(=O)[nH]1]

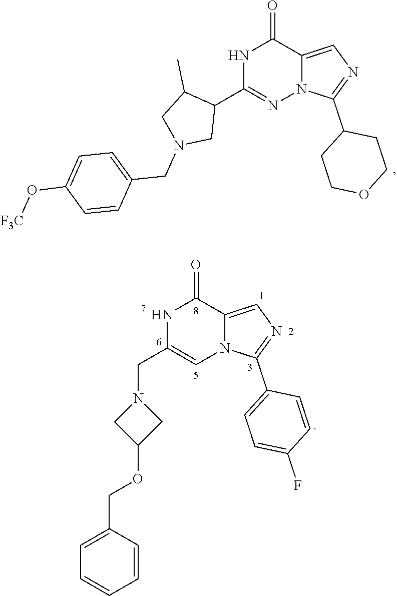 [SMILES: CC1CN(Cc2ccc(OC(F)(F)F)cc2)CC1c1nn2c(C3CCOCC3)ncc2c(=O)[nH]1 ; O=c1[nH]c(CN2CC(OCc3ccccc3)C2)cn2c(-c3ccc(F)cc3)ncc12]

Embodiment 17. The method of any one of embodiments 1-12, wherein the PDE9 inhibitor is represented by a compound of formula (I),

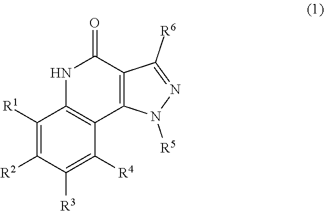

wherein
R¹ is a hydrogen atom;
R² is an aromatic ring group selected from the group consisting of a phenyl group, a pyridinyl group, and a pyrimidinyl group, where the two atoms on the aromatic ring which are adjacent to the carbon atom attached to the pyrazolo[4,3-c]quinoline ring each independently has a substituent selected from Group A1, and the other atoms on the aromatic ring independently optionally have a substituent selected from Group B1;
R³ is a hydrogen atom or a fluorine atom;
R⁴ is a hydrogen atom;
R⁵ is an oxepanyl group, a dioxepanyl group, a tetrahydropyranyl group, or a tetrahydrofuranyl group optionally having a methoxy group;
R⁶ is a hydrogen atom;
Group A1 consists of a halogen atom, a C₁₋₆ alkyl group optionally having 1 to 3 halogen atoms, and a C₁₋₆ alkoxy group; and
Group B1 consists of a halogen atom, a cyano group, a C₁₋₆ alkyl group optionally having 1 to 3 halogen atoms, a C₁₋₆ alkoxy-C₁₋₆ alkyl group, a C₁₋₆ alkoxy group optionally having 1 to 3 halogen atoms, and a tetrahydropyranyl group,
with the proviso that when R² is a 3-pyridinyl group, the substituent at the 4-position is a halogen atom, or a C₁₋₆ alkyl group optionally having 1 to 3 halogen atoms.
   Embodiment 18. The method of any one of embodiments 1-13, wherein the PDE9 inhibitor is a compound having a structure of:

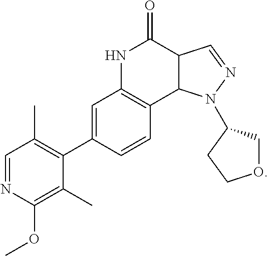 [SMILES: COc1ncc(C)c(-c2ccc3c(c2)NC(=O)C2C=NN([C@H]4CCOC4)C32)c1C]

Embodiment 19. The method of any one of embodiments 1-12, wherein the PDE9 inhibitor is an N-substituted pyrazolo[3,4-d]pyrimidinone compound, with structure of formula (II),

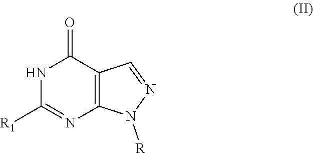

wherein, R is a cyclic or acyclic aliphatic alkyl group, a heterocyclic group, an acyl group, a hydroxyl group, or a mercapto group; R₁ is methoxy, halogen, trifluoromethyl, ethoxy acetyl, cyano, nitro, N, N-dimethyl, chloromethyl, benzyloxy, substituted or unsubstituted amino, substituted guanidino, substituted or unsubstituted phosphoric acid, substituted or unsubstituted sulfonic acid Base, long-chain fatty alkyl, long-chain fatty amino; when R is cyclopentyl, R₁ is not substituted or unsubstituted amino.
   Embodiment 20. The method of any one of embodiments 1-12, wherein the PDE9 inhibitor is represented by a crystal form of compound of formula (III),

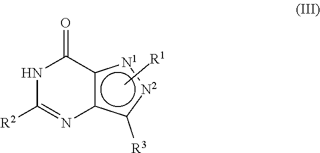

wherein
R¹ is H or C₁₋₆ alkyl, wherein R¹ is attached to either N¹ or N²;
R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹;
R³ is C₁₋₆ alkyl optionally substituted by 1 or 2 groups independently selected from: Ar²; C₃₋₇cycloalkyl optionally substituted by C₁₋₆ alkyl; OAr²; SAr²; NHC(O) C₁₋₆ alkyl; het²; xanthene; and naphthalene; wherein Ar¹ and Ar² are independently groups of formula

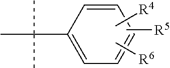

wherein R⁴, R⁵ and R⁶ are independently selected from: hydrogen, halo, phenoxy, phenyl, CF₃, OCF₃, R⁷, SR⁷ and OR⁷,
   wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆ alkoxy, halo and phenyl optionally substituted by 1, 2 or 3 groups independently selected from halo and C₁₋₆ alkyl; with the provisos that when a) R¹ is attached to N¹, R¹ is C₁₋₃ alkyl and R² is propyl then R³ is not methyl substituted by Ar¹, and b) R¹ is attached to N¹, R¹ is C₁₋₆ alkyl and R² is methyl then R³ is not C₁₋₄ alkyl substituted by Ar¹.
      Embodiment 21. The method of any one of embodiments 1-12, wherein the PDE9 inhibitor is represented by a crystal form of compound of formula (IV), 6-ethyl-4-(4-methoxy-4-methylpiperidin-1-yl)-2-oxo-1,2-dihydro-1,7-two,

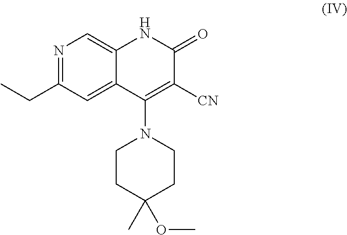 [SMILES: CCc1cc2c(N3CCC(C)(OC)CC3)c(C#N)c(=O)[nH]c2cn1]

wherein the crystalline form I of naphthalene-3-carbonitrile is characterized in that it uses Cu-Kα radiation and X-ray powder diffraction expressed in 2θ angles at 7.3±0.2°, 13.6±0.2°, 14.5±0.2°, There are characteristic peaks at 18.0±0.2°, 19.1±0.2°, 22.0±0.2°, 23.4±0.2°.
   Embodiment 22. The method of any one of embodiments 1-12, wherein the PDE9 inhibitor is represented by a compound of formula (V),

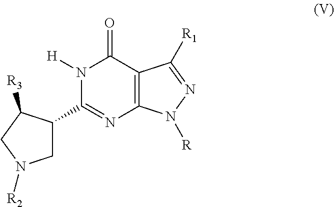

and pharmaceutically acceptable salts thereof, wherein:
   R is selected from the group consisting of (C₁-C₆)alkyl, (C₂-C₆)alkenyl, (C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl.
   R₁ is selected from the group consisting of hydrogen, (C₁-C₄)alkyl, (C₂-C₄)alkenyl, (C₂-C₄)alkynyl, (C₁-C₄)haloalkyl, and cyclopropyl;
   R² is selected from the group consisting of (C₁-C₆)alkyl, (C₂-C₆)alkenyl, (C₂-C₆)alkynyl, (C₁-C₆)haloalkyl, heteroaryl selected from the group consisting of pyridinyl, pyridazinyl, pyrimidinyl, and pyrazinyl, and ER₅, wherein the heteroaryl optionally may be substituted with one to three substituents independently selected from the group consisting of (C₁-C₄)alkyl and (C₁-C₄)haloalkyl;
   R³ is selected from the group consisting of hydrogen, (C₁-C₄)alkyl, (C₁-C₄)alkenyl, (C₂-C₄)alkynyl, (C₃-C₆)cycloalkyl, and (C₁-C₄)haloalkyl;
   E is selected from the group consisting of —CH₂—, —CH₂CH₂—, —CH₂CH₂CH₂—, and —C(O)—;
   R₅ is selected from the group consisting of (C₃-C₈)cycloalkyl, heterocycloalkyl, aryl, aryloxy, and heteroaryl, any of which optionally may be substituted with one to three substituents, such substituents being independently selected from the group consisting of (C₁-C₄)alkyl, (C₂-C₄)alkenyl, (C₂-C₄)alkynyl, (C₁-C₄)hydroxyalkyl, (C₁-C₄)haloalkyl, (C₁-C₄)alkoxy, (C₁-C₄)haloalkoxy, (C₃-C₈)cycloalkyl, halo, cyano, phenyl, morpholinyl, (C₁-C₄)alkylamino, pyrazolyl, triazolyl, and imidazolyl.
      Embodiment 23. The method of embodiment 22, wherein the PDE9 inhibitor is 6-[(3S,4S)-4-methyl-1-(pyrimidin-2-ylmethyl)pyrrolidin-3-yl]-1-(tetrahydro-2H-pyran-4-yl)-1,5-dihydro-4H-pyrazolo[3,4-d]pyrimidin-4-one,

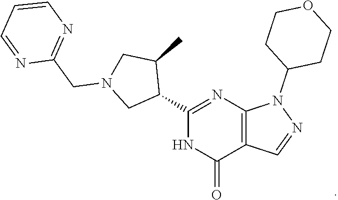 [SMILES: C[C@@H]1CN(Cc2ncccn2)C[C@H]1c1nc2c(cnn2C2CCOCC2)c(=O)[nH]1]

Embodiment 24. The method of any one of embodiments 1-12, wherein the PDE9 inhibitor is represented by a compound of formula (VI), or its pharmaceutically acceptable salt, solvate, polymorph and isomer,

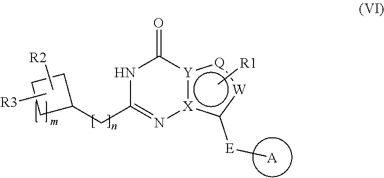

wherein,
m is arbitrarily selected from 0, 1, 2, 3 and 4;
n is arbitrarily selected from 0, 1 and 2;
X and Y are independently selected from C, CH, and N;
Q and W are independently selected from C, CH, N, NH, S and 0;
E is arbitrarily selected from —(CH₂)_{z}—, —O—, —S—, —NH—, and —N(CH₃)—, wherein, z is independently selected from 0, 1 and 2;
R₁ is independently selected from hydrogen, C₁₋₄ alkyl, C₂₋₄ alkenyl, C₂₋₄ alkynyl, C₃₋₆ cycloalkyl, halogenated C₁₋₄ alkyl, and —(CH₂)m′-C₃₋₆ cycloalkyl, m′=1 or 2;
R² and R³ are independently selected from hydrogen, hydroxyl, amino, carboxyl, cyano, nitro, halogen atom, C₁₋₆ alkyl, C₃₋₆ cycloalkyl, C₁₋₆ alkoxy, C₃₋₆ cycloalkyloxy, halogenated C₁₋₆ alkoxy, halogenated C₁₋₆ alkyl, halogenated C₃₋₆ cycloalkyl, C₂₋₈ alkenyl, C₂₋₈ alkyne group, amino C₁₋₆ alkyl, C₃₋₆ cycloalkylamino, C₁₋₆ alkylsulfonyl, 3-8 membered cycloalkylsulfonyl, C₁₋₆ alkylcarbonyl, C₃₋₆ Cycloalkylcarbonyl, C₁₋₆ alkylthio, —(CH₂)n′-5-14 membered cycloalkyl, —(CH₂)n′-5-14 membered aromatic ring, —(CH₂) n′-5-10 membered heterocyclic group containing 1 to 3 O, S and/or N atoms, —(CH₂)n′-5-10 membered heterocyclic group containing 1 to 3 O, S and/or N atoms, wherein, n′=0, 1 or 2, cycloalkyl, aromatic ring, heteroaryl ring, heterocyclyl is optionally substituted by 1-3 R⁴;
R⁴ is independently selected from hydrogen, hydroxyl, amino, carboxyl, cyano, nitro, halogen atom, C₁₋₄ alkyl, trifluoromethyl, methoxy, cyclopropyl, —(CH₂)m′-C₃₋₆ cycloalkyl, m′=1 or 2;
Ring A is selected from optional 5-10 membered cycloalkyl, 5-10 membered aromatic ring, 5-10 membered heterocycloalkyl containing 1-3 O, S and/or N atoms, wherein, ring A is optionally substituted by 1-3 R5, any ring atom S is optionally oxidized to S(O) or S(O)₂, any ring atom carbon can be optionally oxidized to C(O);
R⁵ is independently selected from hydrogen, hydroxyl, amino, carboxyl, cyano, nitro, halogen atom, C₁₋₆ alkyl, C₁₋₆ alkoxy, methoxy, trifluoromethyl, cyclopropyl, —(CH₂)m′—C₃₋₆ cycloalkyl, m′=1 or 2.
   Embodiment 25. The method of any one of embodiments 1-12, wherein the PDE9 inhibitor is represented by a compound of formula (VII),

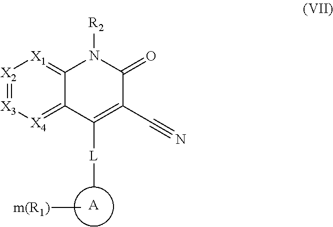

wherein, X₁, X₂, X₃, and X₄ are independently selected from CR₃ or N, and N heteroatoms can be optionally oxidized to

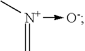

R³ at each occurrence is independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halo C₁₋₆ alkyl, halo C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂ amino, halo C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy are not substituted or optionally substituted with one or more groups independently selected from hydroxyl, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₂₋₈ alkynyl, halo C₁₋₆ alkyl, C₂₋₈ alkenyl, halo C₁₋₆ alkoxy, 4-6 membered heterocyclyl unsubstituted or optionally substituted with a substituent, and heteroaryl unsubstituted or optionally substituted with a substituent;
the substituents of the above-mentioned 4-6 membered heterocyclyl optionally substituted with a substituent and heteroaryl optionally substituted with a substituent are selected from hydroxyl, amino, carboxyl, cyano, nitro, halogen, C₁₋₆ alkyl and C₁₋₆ alkoxy;
L is a bond or —NH—(CH₂)ₜ—, and t is 0, 1, 2 or 3;
ring A is 3-12 membered heterocyclyl, aryl, 5-10 membered heteroaryl, 3-12 membered cycloalkyl, and 3-12 membered cycloalkenyl, wherein the heteroatom of the 3-12 membered heterocyclyl is selected from one of O, S, and N or any combination thereof, the S atom may be optionally oxidized to S(O) or S(O)₂, the C atom may be optionally oxidized to C(O), and the N heteroatom may be optionally oxidized to

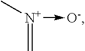

and the heteroatom of the 5-10 membered heteroaryl is selected from one of O, S and N or any combination thereof;
each R₁ is independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halo C₁₋₆ alkyl, halo C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halo
C₁₋₆ alkyl, halo C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are not substituted or optionally substituted with groups selected from hydroxyl, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ alkylsulfonylamino;
m is 0, 1, 2 or 3; and
R₂ is selected from hydrogen, C₁₋₆ alkyl, C₂₋₈ alkenyl, C₂₋₈ alkynyl, and halo C₁₋₆ alkyl.
   Embodiment 26. The method of any one of embodiments 1-12, wherein the PDE9 inhibitor is represented by a compound of formula (VIII) or its is pharmaceutically acceptable salt, solvated compounds, polymorph and isomers;

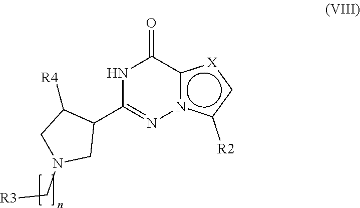

wherein,
n is selected from 0, 1 or 2;
X is selected from N or CR₁;
R₁ is independently selected from hydrogen, C₁₋₆Alkyl, C₂₋₈Alkenyl, C₂₋₈Alkynyl, C₃₋₆ naphthenic base, halogenated C₁₋₆Alkyl, —(CH₂)_{m′}—C₃₋₆naphthenic base, m′=1 or 2;
R² is independently selected from hydrogen, C₁₋₆Alkyl, C₁₋₆Alkoxy, halogenated C₁₋₆Alkyl, halogenated C₁₋₆Alkoxy, C₃₋₆Naphthenic base Oxygroup, C₃₋₆Cycloalkyl amino, halogenated C₃₋₆Naphthenic base, C₂₋₈Alkenyl, C₂₋₈Alkynyl, C₁₋₆Alkyl sulphonyl, C₃₋₈First naphthenic base sulphur Acyl group, C₁₋₆Alkyl sulfenyl, C₂₋₈Alkenyl (C₃₋₆) naphthenic base, C₂₋₈Alkynyl (C₃₋₆) naphthenic base, C₁₋₆Alkyl-carbonyl, C₃₋₆Naphthenic base Carbonyl, —(CH₂)_{n′}—3-14 members naphthenic base, —(CH₂)_{n′}—5-14 members aromatic ring, —(CH₂)_{n′}—5-10 circle heterocyclic rings base, —(CH₂)_{n′}-5-10 unit's heteroaryls, the heterocycle, heteroaryl contain 1-3 O, S and/or N atom, wherein the sulfur of any ring atom of the heterocyclyl group or heteroaryl group is optionally oxidized to S(O) or S(O)₂, any ring atom carbon is optionally oxidized to C(O), n′=0, 1 or 2, cycloalkyl, heterocyclyl aromatic ring, heteroaromatic ring is optionally replaced by 1-3 R₅;
wherein, when X is selected from CR1, and R¹ is hydrogen, R² cannot be

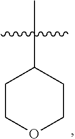 [SMILES: *C1CCOCC1]

R₃ is independently selected from
hydrogen, hydroxyl, amino, carboxyl, cyano, nitro, halogen atom, C₁₋₆ alkyl, C₃₋₆ cycloalkyl, C₁₋₆ alkoxy, C₃₋₆ Cycloalkyloxy, halogenated C₁₋₆ alkoxy, halogenated C₁₋₆ alkyl, halogenated C₃₋₆ cycloalkyl, C₂₋₈ alkenyl, C₂₋₈ alkynyl, amino C₁₋₆ alkyl, C₃₋₆ cycloalkylamino, C₁₋₆ alkylsulfonyl, 3-8 membered cycloalkylsulfonyl, C₁₋₆ alkylcarbonyl, C₃₋₆ cycloalkyl Carbonyl, C₁₋₆ alkylthio, 3-14 membered cycloalkyl, 5-14 membered aromatic ring, -5-10 membered heterocyclic group, -5-10 membered heteroaryl group, said heterocyclic group, the heteroaryl group contains 1-3 O, S and/or N atoms, wherein, any ring atom sulfur of the heterocyclic group and the heteroaryl group is optionally oxidized to S(O) or S(O)₂, any ring atomic carbon is optionally oxidized to C(O), cycloalkyl, aromatic ring, heteroaryl ring, heterocyclyl is optionally substituted by 1-3 R₆;
R⁴ is independently selected from hydrogen, hydroxyl, amino, carboxyl, cyano, nitro, halogen atom, C₁₋₆alkyl, C₁₋₆alkoxy, C₃₋₆cycloalkyl oxy, halogenated C₁₋₆alkoxy, halogenated C₁₋₆alkyl, halogenated C₃₋₆naphthenic base, C₂₋₆alkenyl, C₂₋₆alkynyl, amino C₁₋₆ alkyl, C₃₋₆cycloalkyl amino, C₁₋₆alkyl sulphonyl, 3-8 membered rings alkyl sulphonyl, C₁₋₆alkyl-carbonyl or
C₃₋₆Naphthenic base carbonyl Base, C₁₋₆alkyl sulfenyl or —(CH₂)_{n′}—3-7 members naphthenic base, —(CH₂)_{n′}—5-6 members aromatic ring, —(CH₂)_{n′}-5-7 circle heterocyclic rings base, —(CH₂)_{n′}-5-6 unit's heteroaryls, the heterocycle, heteroaryl contain 1-3 O, S and/or N atom, wherein any ring S of the heterocyclyl or heteroaryl is optionally oxidized to S(O) or S(O)₂, arbitrary ring carbon is optionally oxidized to C(O), n′=0, 1 or 2, cycloalkyl ring, aromatic ring, hetero-aromatic ring, heterocycle is optionally replaced by 1-3 R₇;
R⁵ is independently selected from hydrogen, hydroxyl, amino, carboxyl, cyano, nitro, halogen atom, trifluoromethyl, methoxyl group, —(CH₂)_{n′}—3-14 members naphthenic base, —(CH₂)_{n′}-5-14 members aromatic ring, —(CH₂)_{n′}—5-10 circle heterocyclic rings base, —(CH₂)_{n′}-5-10 members Heteroaryl, the heterocycle, heteroaryl contain 0-3 O, S and/or N atom, wherein n′=0, 1 or 2;
R⁶ and R⁷ are independently selected from hydrogen, hydroxyl, amino, carboxyl, cyano, nitro, halogen atom, trifluoromethyl, C₁₋₆Alkyl, C₁₋₄Alkoxy, C₁₋₆Alkyl sulphonyl, C₁₋₆Alkyl sulphonyl C₁₋₆Alkyl, C₁₋₆Alkyl sulphonyl C₁₋₆Alkoxy, aminosulfonyl Amino C₁₋₆Alkyl, methoxyl group, cyclopropyl, —(CH₂)m′—C₃₋₆Naphthenic base, m′=1 or 2.
   Embodiment 27. The method of any one of embodiments 1-12, wherein the PDE9 inhibitor is represented by a compound of formula (X),

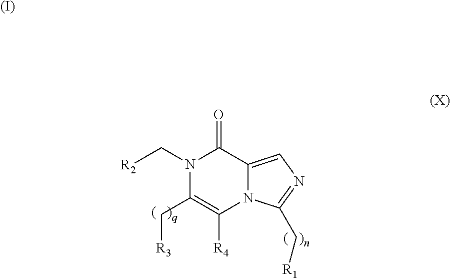

wherein:
   n is 0 or 1;
   q is 0 or 1;
   R₁ is selected from the group consisting of benzyl, indanyl, indoline and 5-membered heteroaryls; all of which can be substituted with a substituent selected from the group consisting of halogen and C₁-C₃ alkyl; or
   R₁ is selected from the group consisting of saturated monocyclic rings containing 4-6 carbon atoms and 1-2 nitrogen atoms; all of which can be substituted one or more times with one or more substituents selected from the group consisting of methyl, fluorine and sulfonamide; or
   R₁ is selected from the group consisting of lactams containing 4-6 carbon atoms; all of which can be substituted one or more times with one or more substituents selected from the group consisting of methyl and fluorine; or
   R₁ is selected from the group consisting of bicyclic ethers such as,
   7-oxabicyclo[2.2.1]heptane; all of which can be substituted one or more times with one or more substituents selected from the group consisting of methyl and fluorine; or
   R₁ is selected from the group consisting of linear or branched C₁-C₈ alkyl, saturated monocyclic C₃-C₅ cycloalkyl, oxetanyl, tetrahydrofuranyl and tetrathydropyranyl; all of which can be substituted one or more times with one or more substituents selected from the group consisting of methyl, fluorine, hydroxy, cyano or methoxy; or
   R₁ is a linear or branched C₁-C₃ alkyl, which is substituted with a substituent selected from phenyl and 5-membered heteroaryl, wherein said 5-membered heteroaryl can be substituted with one or more C₁-C₃ alkyls; or
R₁ is selected from the group consisting of morpholine, tetrahydrofuran-3-amine, hexahydro-2H-furo[3,2-b]pyrrole and homomorpholine; all of which can be subsituted with one or more substituents selected from the group consisting of C₁-C₃ alkyl;
   R₂ is selected from the group consisting of hydrogen, linear or branched C₁-C₈ alkyl, phenyl, saturated monocyclic C₃-C₈ cycloalkyl, oxetanyl, benzo[d][1,3]dioxolyl, tetrahydrofuranyl and tetrahydropyranyl; or
   R₂ is phenyl or pyridyl substituted with one or more substituents selected from the group consisting of hydroxyl, amino, cyano, halogen, C₁-C₃ alkyl, C₁-C₃ alkoxy, C₃-C₅ cycloalkoxy, C₃-C₅ cycloalkyl-methoxy, C₁-C₃ fluoroalkoxy, and —NC(O)CH₃; or
   R₂ is a 5-membered heteroaryl which can be substituted one or more times with C₁-C₃ alkyl;
   R³ is selected from the group consisting of hydrogen, halogen, C₁—C alkyl, C₃-C₅ cycloalkyl and phenyl; or
   R₃ is selected from the group consisting of phenyl substituted one or more times with C₁-C₃ alkyl; methyl substituted one, two or three times with fluorine; ethyl substituted one, two or three times with fluorine;
   R₄ is hydrogen;
and tautomers and pharmaceutically acceptable addition salts thereof;
   with the proviso that R² and R³ cannot be hydrogen at the same time;
   with the proviso, that the compound of formula (I) is not one of the three following compounds:
3-methyl-7-(4-(trifluoromethoxy)benzyl)imidazo[1,5-a]pyrazin-8(7H)-one;
7-butyl-3-methylimidazo[1,5-a]pyrazin-8(7H)-one; and
7-(4-methoxybenzyl)-3-methylimidazo[1,5-a]pyrazin-8(7H)-one.
   Embodiment 28. The method of any one of embodiments 1-12, wherein the PDE9 inhibitor is represented by a compound of formula (XI),

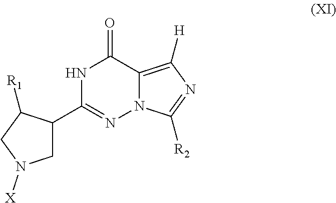

wherein:
X is selected from the group consisting of a bond, C(O) and S(O)₂;
R₁ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and
heterocycloalkyloxy, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl];
R₂ is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl]; and
R₃ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl, —S(O)₂NH(C₁-C₄)alkyl, and —S(O)₂N[(C₁-C₄)alkyl(C₁-C₄)alkyl].
   Embodiment 29. The method of any one of embodiments 1-12, wherein the PDE9 inhibitor is represented by a compound of formula (XII),

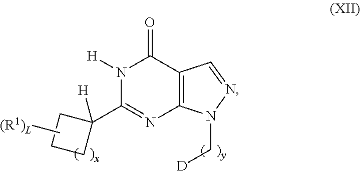

wherein
   R¹ is selected independently for each R¹ from the group R^{1a} consisting of hydrogen, fluorine, chlorine, bromine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, heterocyclyl-CO—, R¹⁰—O—, R¹⁰—O—C₁₋₃-alkyl-, (R¹⁰)₂N—, R¹⁰—CO—, (R⁹)₂N—CO—, R¹⁰—CO—(R¹⁰)N—, R¹⁰—CO—, (R⁹)₂N—CO—(R¹⁰)N—, (R⁹)₂N—CO—O—, R¹⁰—O—CO—(R¹⁰)N—, R¹⁰—SO₂—(R¹⁰)N—, and C₁₋₆-alkyl-SO₂—,
      where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, heterocyclyl-CO—, and C₁₋₆-alkyl-SO₂— may optionally be substituted independently of one another by one or more substituents selected independently of one another from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, C₃₋₆-cycloalkyl-C₁₋₄-alkyl-, and C₁₋₆-alkyl-, preferably from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, and C₃₋₆-cycloalkyl-C₁₋₄-alkyl-,
   L is selected from the integers 0, 1, 2 and 3,
   x is selected from the integers 0, 1, 2, 3 and 4,
   y is selected from the integers 0, 1 and 2,
   D is selected from the group D^{1a} consisting of heterocyclyl,
      wherein the above-mentioned members of the group D^{1a} may optionally be substituted by one or more substituents selected independently of one another from the group R² and/or optionally substituted by one group R³
      or
   D is selected from the group D^{2a} consisting of cyclopropyl, cyclobutyl, cyclopentyl, cyclohexyl, cycloheptyl cyclooctyl, cyclobutenyl, cyclopentenyl, cyclohexenyl, cycloheptenyl, cyclooctenyl, cyclopentadienyl, cyclohexadienyl, cycloheptadienyl, cyclooctadienyl, cycloheptatrienyl, cyclooctatrienyl and cyclooctatetraenyl, wherein the above-mentioned members of the group D^{2a} may optionally be substituted by one or more substituents selected independently of one another from the group R⁴,
      or
   D is selected from the group D^{3a} consisting of C₁₋₈-alkyl
      wherein the above-mentioned C₁₋₈-alkyl-group D^{3a} may optionally be substituted by one or more substituents selected independently of one another from the group R⁵.
      or
   D is selected from the group D^{4a} consisting of aryl
      wherein the above-mentioned aryl group D^{4a} may optionally be substituted by one or more substituents selected independently of one another from the group consisting of R⁶. Preferred are such compounds wherein D^{4a} is substituted by not more than one R⁶.
      or
   D is selected from the group D^{5a} consisting of heteroaryl
      wherein the above-mentioned members of the group D^{5a} may optionally be substituted by one or more substituents selected independently of one another from the group R⁶. Preferred are such compounds wherein D^{5a} is substituted by not more than one R⁶.
   R² is selected from the group R^{2a} consisting of
      H—, fluorine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, C₁₋₆-alkyl-SO₂—, and oxo,
      where the above-mentioned members HF₂C—, FH₂C—, F₃C—CH₂—, C₁₋₆-alkyl-(Preferably C₂₋₆-alkyl), C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—C₁₋₃-alkyl-, and C₁₋₆-alkyl-SO₂— may optionally be substituted independently of one another by one or more substituents selected independently of one another from the group consisting of
      fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—, and in cases in that D¹ is a heterocyclyl group with NR² as ring member, R² shall be
         independently of any other R²: H—, F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, R¹⁰O—CO—, (R¹⁰)₂N—CO—, R¹⁰—CO—, R¹⁰—SO₂—, and C₁₋₆-alkyl-SO₂—, where the above-mentioned members F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, and C₁₋₆-alkyl-SO₂— may optionally be substituted independently of one another by one or more substituents selected independently of one another from the group consisting of
         fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and (R¹⁰)₂N—CO—,
   R³ is selected from the group R^{3a} consisting of
      H—, HO— and R¹⁰—O—,
   R⁴ is selected from the group R^{4a} consisting of
      H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—, where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃-7-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
      or
      two substituents R^{4a} together form a C₂₋₆-alkylene bridge, wherein one or two CH₂ groups of the C₂₋₆-alkylene bridge may be replaced independently of one another by O, S, SO, SO₂, N(R¹⁰) or N—C(O)—R¹⁰ in such a way that in each case two O or S atoms or an 0 and an S atom are not joined together directly.
      R⁵ is selected from the group R^{5a} consisting of
      H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
      where the above-mentioned members HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂—, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
   R⁶ is selected from the group R^{6a} consisting of
      H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰—CO—, (R¹⁰)₂N—CO—, R¹⁰—CO—(R¹⁰)N—, R¹⁰—CO—, (R¹⁰)₂N—CO—(R¹⁰)N—, R¹⁰—CO—(R¹⁰)N—, R¹⁰—SO₂—(R¹⁰)N—, and C₁₋₆-alkyl-SO₂—,
      where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl- and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
   R⁹ is selected independently for each R⁹ from the group R^{9a} consisting of H—F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, and
      where the above-mentioned members F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, may optionally be substituted independently of one another by one or more substituents selected independently of one another from the group consisting of
      fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl-O— and C₁₋₆-alkyl-,
   R¹⁰ is selected independently for each R¹⁰ from the group R^{10a} consisting of H— (but not in case is part of a group being selected from R¹⁰—CO—, R¹⁰—SO₂— or R¹⁰—CO—), F₃C—CH₂—, C₁₋₆-alkyl-, C₂₋₆-alkenyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, and heteroaryl-C₁₋₃-alkyl-,
      and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —S—, —NH—, —N(C₃₋₆-cycloalkyl)-, —N(C₃₋₆-cycloalkyl-C₁₋₄-alkyl)- or —N(C₁₋₄-alkyl)-
      and
      where the above-mentioned members may optionally be substituted independently of one another by one or more substituents selected from the group consisting of
      fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl- and C₁₋₆-alkyl-O—
      and pharmaceutically acceptable salt forms or solvates thereof.
         Embodiment 30. The method of any one of embodiments 1-12, wherein the PDE9 inhibitor is represented by a compound of formula (XIII),

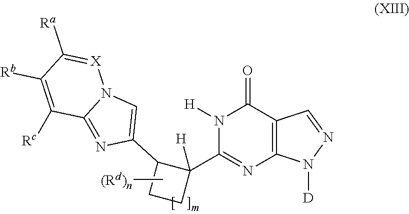

wherein
X is N or CR^{c},
R^{a}, R^{b}, R^{c}, R^{e} are selected independently of each other from the group consisting of H, C₁₋₆alkyl-, C₁₋₆-alkyl-O—, CF₃—, CHF₂—, CH₂F—, NC— and halogen; whereby C₁₋₆alkyl- and C₁₋₆-alkyl-O—
optionally may be substituted with halogen, preferably with fluoro
R^{d} is selected from the group consisting of fluorine, NC—, —CF₃, —CHF₂, —CH₂F, and methyl;
D is selected from the group consisting of cyclopentyl, cyclohexyl, tetrahydrofuranyl, tetrahydropyranyl, 2-, 3- and 4-pyridyl,
whereby cyclopentyl and cyclohexyl optionally may be substituted by 1 or 2 substituents, whereby said substituents may be selected independently of one another from the group consisting of fluorine, NC—, F₃C—, HF₂C— and FH₂C—;
whereby tetrahydrofuranyl and tetrahydropyranyl optionally may be substituted by 1 or 2 substituents, whereby said substituents may be selected independently of one another from the group consisting of fluorine, NC—, F₃C—, FIF₂C— and FH₂C—;
whereby pyridyl optionally may be substituted by 1, 2, 3 or 4 substituents, whereby said substituents may be selected independently of one another from the group consisting of fluorine, chlorine, bromine, NC—, F₃C—, HF₂C—, FH₂C—; F₃C—CH₂—, C₁₋₆-alkyl- and C₃₋₇-cycloalkyl;
m is selected from 1 or 2, preferably 1;
n is selected from 0, 1 or 2, preferably, 0 or 1, more preferably 0,
whereby if n=2, the two groups R^{d} are selected independently of one another; and salts, preferably pharmaceutically acceptable salts thereof, solvates thereof and the solvates of the aforementioned salts thereof.
   Embodiment 31. The method of any one of embodiments 1-12, wherein the PDE9 inhibitor is represented by a compound of formula (XIV),

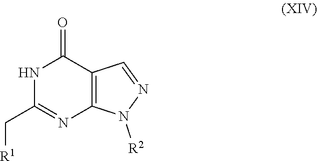

in which
R¹ is phenyl, pyridyl or thiophenyl, which are optionally selected with up to 3 substituents independently of one another from the group of C₁-C₆-alkyl, C₁-C₆-alkoxy, hydroxycarbonyl, cyano, trifluoromethyl, amino, nitro, hydroxy, C₁-C₆Alkylamino, halogen,
C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-Akylsulfonylamino, C₁-C₆ Alkylsulfonyl, C₁-C₆ alkylthio, wherein C₁-C₆-alkyl, C₁-C₆-alkoxy, C₆-C₁₀ alkylamino, C₆-C₁₀-Axylcarbonylamino, C₁-C₆-Alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulfonylamino, C₁-C₆-alkylsulfonyl and C₁-C₆-alkylthio optionally with a residue Group hydroxy, cyano, halogen, hydroxy, carbonyl and a group of the formula —NR³R⁴,
in which
R³ and R⁴ independently of one another are hydrogen or C₁-C₆-alkyl,
or
R³ and R⁴ together with the nitrogen atom to which they are attached, 5- to 8-membered heterocyclyl,
R² is phenyl or heteroaryl, phenyl having 1 to 3 residues and heteroaryl optionally having 1 to 3 residues, each independently selected from the group C₁-C₆ alkyl, C₁-C₆ alkoxy, hydroxycarbonyl, cyano, trifluoromethyl, amino, nitro, hydroxy, C₁-C₆ alkylamino, halogen, C₆-C₁₀ arylcarbonylamino, C₁-C₆ alkylcarbonylamino, C₁-C₆ alkylaminocarbonyl, C₁-C₆ alkoxycarbonyl, C₆-C₁₀ arylaminocarbonyl, heteroarylaminocarbonyl, heteroarylcarbonylamino, C₁-C₆ alkylsulfonylamino, and C₁-C₆-alkylthio, wherein C₁-C₆-alkyl, C₁-C₆-alkoxy, C₁-C₆ alkylamino, C₆-C₁₀-arylcarbonylamino, C₁-C₆-alkylcarbonylamino, C₁-C₆ alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulfonylamino, C₁-C₆ alkylsulfonyl and C₁-C₆ alkylthio optionally with a radical selected from the group consisting of hydroxy, cyano, halogen, hydroxycarbonyl and a group of the formula —NR³R⁴,
where R³ and R⁴ have the meanings given above,
or their salts, solvates and/or solvates of the salts.
   Embodiment 32. The method of any one of embodiments 1-12, wherein the PDE9 inhibitor is represented by a compound of formula (XV),

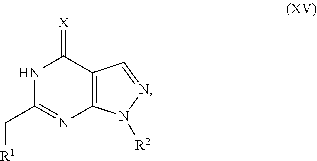

in which
   R₁ is phenyl which is substituted by 1 to 5 substituents independently of one another selected from the group of halogen, C₁-C₆-alkyl, trifluoromethyl, trifluoromethoxy, cyano, hydroxy, nitro and C₁-C₆-alkoxy,
   R² is pentan-3-yl, C₄-C₆-cycloalkyl,
   X is oxygen or sulfur,
   and the salts, solvates and/or solvates of the salts thereof.
      Embodiment 33. The method of any one of embodiments 1-12, wherein the PDE9 inhibitor is represented by a compound of formula (XVI),

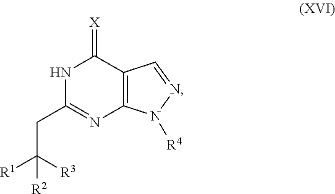

in which
   R¹ is C₁-C₆-alkyl, trifluoromethyl, hydroxy, C₁-C₆-alkoxy, —C(═O)OR⁵ or —C(═O)NR⁶R⁷, where C₁-C₆-Alkyl is optionally substituted by 1 to 3 radicals independently of one another selected from the group of hydroxy, C₁-C₆-alkoxy, halogen, trifluoromethyl, trifluoromethoxy, —C(═O)OR⁵ or —C(═O)NR⁶R⁷, and
      R⁵ is C₁-C₆-alkyl,
      R⁶ and R⁷ are independently of one another hydrogen, C₆-C₁₀-aryl, C₁-C₆-alkyl, or
         together with the nitrogen atom to which they are bonded form a 4- to 10-membered heterocyclyl,
   R² is hydrogen, C₁-C₆-alkyl, trifluoromethyl, C₁-C₆-alkoxy,
   or
   R¹ and R² together with the carbon atom to which they are bonded form C₃-C₈-cycloalkyl, C₃-C₈-cycloalkenyl or 4- to 10-membered heterocyclyl, which are optionally substituted by up to 2 substituents from the group of C₁-C₆-alkyl, C₁-C₆-alkoxy, hydroxy, oxo, —C(═O)OR⁸, and R⁸ is C₁-C₆-alkyl or benzyl,
   R³ is hydrogen or C₁-C₆-alkyl,
   R⁴ is pentan-3-yl, C₃-C₆-cycloalkyl,
   X is oxygen or sulfur,
   and the salts, solvates and/or solvates of the salts thereof.
      Embodiment 34. The method of any one of embodiments 1-12, wherein the PDE9 inhibitor is represented by a compound of formula (XVII),

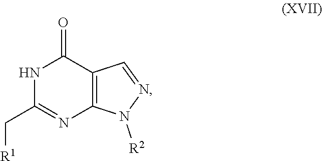

with:
   R¹
   being phenyl or pyridyl, any of which is substituted with 1 to 4 substituents X; and with the option that each of phenyl or pyridyl in addition may be substituted by up to 3 radicals independently of one another selected from the group of C₁-C₆-alkyl, C₁-C₆-alkoxy, hydroxycarbonyl, cyano, trifluoromethyl, amino, nitro, hydroxy, C₁-C₆-alkylamino, halogen, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylamino-carbonyl, heteroarylcarbonylamino, C₁-C₆-alkylsulphonylamino, C₁-C₆-alkylsulphonyl, or C₁-C₆-alkylthio,
   where each of C₁-C₆-alkyl, C₁-C₆-alkoxy, C₁-C₆-alkylamino, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio are optionally substituted by 1 to 3 radicals independently of one another selected from the group of hydroxy, cyano, halogen, hydroxycarbonyl and a group of the formula —NR³R⁴,
   X
   independently of each other being selected from C₁-C₆-alkoxy, where the C₁-C₆-alkoxy is substituted with 2 to 6 halogen substituents, and the halogen substituents are selected from the group of fluoro, chloro and bromo, whereby the C-atom directly attached to the 0-atom, which constitutes the beta position with respect to the link to the phenyl or pyridyl, is substituted with at least one halogen atom;
   R²
   being phenyl or heteroaryl, where phenyl is substituted by 1 to 3 radicals and heteroaryl is optionally substituted by 1 to 3 radicals in each case independently of one another selected from the group of C₁-C₆-alkyl, C₁-C₆-alkoxy, hydroxycarbonyl, cyano, trifluoromethyl, amino, nitro, hydroxy, C₁-C₆-alkylamino, halogen, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio,
   where each of C₁-C₆-alkyl, C₁-C₆-alkoxy, C₁-C₆-alkylamino, C₆-C₁₀-arylcarbonylamino, C₁-C₆-alkylcarbonylamino, C₁-C₆-alkylaminocarbonyl, C₁-C₆-alkoxycarbonyl, C₆-C₁₀-arylaminocarbonyl, heteroarylaminocarbonyl, heteroarylcarbonylamino, C₁-C₆-alkylsulphonylamino, C₁-C₆-alkylsulphonyl and C₁-C₆-alkylthio are optionally substituted by one to three radicals independently of one another selected from the group of hydroxy, cyano, halogen, hydroxycarbonyl and a group of the formula —NR³R⁴,
   R³
   being hydrogen or C₁-C₆-alkyl,
   and R⁴
   being hydrogen or C₁-C₆-alkyl,
   or R³ and R⁴ together with the nitrogen atom to which they are bonded are 5- to 8-membered heterocyclyl.
      Embodiment 35. The method of any one of embodiments 1-12, wherein the PDE9 inhibitor is represented by a compound of formula (XVIII),

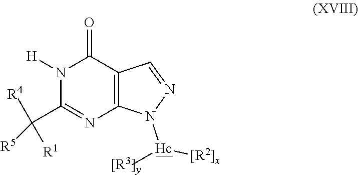

He is selected from the group of tetrahydropyranyl, tetrahydrofuranyl, piperidinyl and pyrrolidinyl;
R¹ is selected from the group of phenyl, 2-, 3- and 4-pyridyl-, pyrimidinyl, pyrazolyl, thiazolyl, cyclopropyl, cyclobutyl, cyclopentyl, cycloheptyl, cyclopentylmethyl, cyclohexyl, ethyl, 1- and 2-propyl, 1- and 2-butyl-, 1-, 2- and 3-pentyl-, tetrahydrofuranyl and tetrahydropyranyl,
   where these R¹ groups may optionally be substituted by one or more substituents independently of one another selected from the group consisting of fluorine, chlorine, bromine, iodine, HO—, oxo, NC—, C₁₋₆-alkyl-O—, C₁₋₆-alkyl-, C₃₋₇-cycloalkyl-O—, C₃₋₇ cycloalkyl-C₁₋₃alkyl-O—, CF₃O— and CF₃;
R² independently of any other R² is selected from the group of H—, C₁₋₆-alkyl- and oxo, and in cases wherein R² is attached to a nitrogen which is a ring member of Hc, this R² shall be independently of any other R² selected from: H—, C₁₋₆-alkyl-CO—, C₁₋₆-alkyl-O—CO—, C₁₋₆-alkyl-, phenyl-CO—, phenyl-O—CO— and (C₁₋₆-alkyl)₂N—CO—,
   where the above-mentioned R² members may optionally be substituted independently of one another by one or more fluorine substituents;
R³ is selected from the group of H—, hydroxy and C₁₋₆-alkyl-O—, whereby C₁₋₆-alkyl-O— may optionally be substituted by one or more fluorine, chlorine, bromine and HO—;
R⁴ and R⁵ independently of one another are selected from the group of H—, fluorine, and methyl;
x is 0, 1, 2, 3 or 4;
y is 0 or 1;
   or a pharmaceutically acceptable salt, a tautomer or a stereoisomer thereof.
   Embodiment 36. The method of embodiment 35 wherein the PDE9 inhibitor is a compound having a structure of:

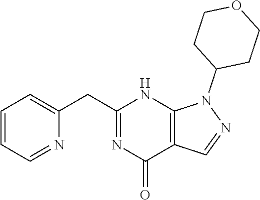 [SMILES: O=c1nc(Cc2ccccn2)[nH]c2c1cnn2C1CCOCC1]

Embodiment 37. The method of any one of embodiments 1-12, wherein the PDE9 inhibitor is represented by a compound of formula (XIX),

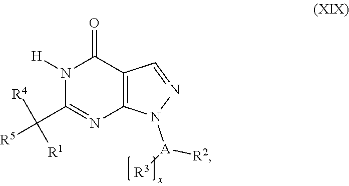

wherein
A is cyclohexyl;
R¹ is selected from the group consisting of phenyl, 2-, 3- and 4-pyridyl, cyclopropyl, cyclobutyl, cyclopentyl, cyclohexyl, ethyl, 1- and 2-propyl, 1- and 2-butyl, 1-, 2- and 3-pentyl, tetrahydrofuranyl and tetrahydropyranyl,
wherein these R¹ groups may optionally be substituted by one or more substituents selected from the group R^{1.1} which consists of fluorine, chlorine, bromine, iodine, NC—, C₁₋₆-alkyl-O—, C₁₋₆-alkyl-, CF₃₀—, F₃C—, pyridyl, (R¹⁰)₂N—CO—CH₂—, N-morpholinyl-C₁₋₆-alkyl-, pyrazolyl and phenyl,
and wherein if R¹ is tetrahydrofuranyl or tetrahydropyranyl, it may also be substituted with oxo,
and wherein the pyridyl, pyrazolyl and phenyl group of the aforementioned group R¹ may optionally be substituted with a group selected from fluorine, chlorine, H₃C—, F₃C—CH₃O—, H₂NCO— and NC—;
R² is fluorine;
R³ is fluorine;
R⁴ and R⁵ are independently selected from H and fluorine;
R¹⁰ independently of any other R¹⁰ is selected from H—, C₁₋₆-alkyl-, phenyl and pyridyl; and
x is 1;
and salts thereof.
   Embodiment 38. The method of any one of embodiments 1-12, wherein the PDE9 inhibitor is represented by a compound of formula (XX),

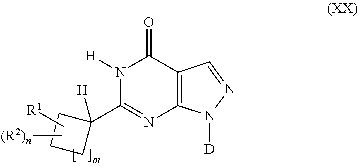

wherein
R¹: is a 5 or 6 membered heteroaryl-group whereby 1, 2, 3 or 4, preferably 1, 2 or 3, of the ring atoms are heteroatoms that are selected independently of each other from N, O or S, whereby said 5 or 6 membered aromatic heteroaryl-group optionally may be substituted by 1, 2, 3 or 4, preferably 1 or 2 substituents, whereby said substituents may be selected independently of one another from the group consisting of fluorine, chlorine, bromine, HO—, NC—, F₃C—, HF₂C—, FH₂C—, methyl, H₂N- and (CH₃)₂N—;
R²: is selected from the group consisting of fluorine, NC—, F₃C—, HF₂C—, FH₂C— and methyl, preferably fluorine, NC—, F₃C— and methyl;
D: is selected from the group consisting of cyclopentyl, cyclohexyl, tetrahydrofuranyl, tetrahydropyranyl, 2-, 3- and 4-pyridyl, whereby cyclopentyl and cyclohexyl optionally may be substituted by 1 or 2 substituents, whereby said substituents may be selected independently of one another from the group consisting of fluorine, NC—, F₃C—, HF₂C— and FH₂C—;
whereby tetrahydrofuranyl and tetrahydropyranyl optionally may be substituted by 1 or 2 substituents, whereby said substituents may be selected independently of one another from the group consisting of fluorine, NC—, F₃C—, FIF₂C— and FH₂C—;
whereby pyridyl optionally may be substituted by 1, 2, 3 or 4 substituents, whereby said substituents may be selected independently of one another from the group consisting of fluorine, chlorine, bromine, NC—, F₃C—, HF₂C—, FH₂C—; F₃C—CH₂—, C₁₋₆-alkyl- and C₃₋₇-cycloalkyl;
m: is selected from 1 or 2, preferably 1;
n: is selected from 0, 1 or 2, preferably, 0 or 1, more preferably 0,
whereby if n=2, these two groups R² are selected independently of one another; and salts, preferably pharmaceutically acceptable salts thereof, solvates thereof and the solvates of the aforementioned salts thereof; with the roviso that the compound is not the following oxadiazolyl-derivative

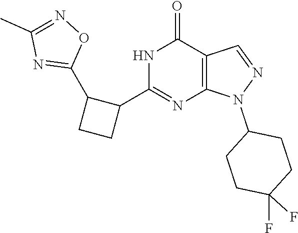 [SMILES: Cc1noc(C2CCC2c2nc3c(cnn3C3CCC(F)(F)CC3)c(=O)[nH]2)n1]

be it in the form of any possible stereoisomer or a mixture of all or some thereof or salt thereof or solvate thereof or a solvate of a salt thereof.
   Embodiment 39. The method of any one of embodiments 1-12, wherein the PDE9 inhibitor is represented by a compound of formula (XXI),

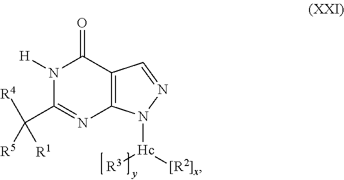

wherein
He is selected from the group of tetrahydropyranyl, tetrahydrofuranyl, piperidinyl and pyrrolidinyl;
R¹ is selected from the group of
phenyl, 2-, 3- and 4-pyridyl-, pyrimidinyl, pyrazolyl, thiazolyl, cyclopropyl, cyclobutyl, cyclopentyl, cycloheptyl, cyclopentylmethyl, cyclohexyl, ethyl, 1- and 2-propyl, 1- and 2-butyl-, 1-, 2- and 3-pentyl-, tetrahydrofuranyl and tetrahydropyranyl,
where these R¹ groups may optionally be substituted by one or more substituents independently of one another selected from the group consisting of fluorine, chlorine, bromine, iodine, HO—, oxo, NC—, C₁₋₆-alkyl-O—, C₁₋₆-alkyl-, C₃₋₇-cycloalkyl-O—, C₃₋₇cycloalkyl-C₁₋₃alkyl-O—, CF₃O— and CF₃;
R² independently of any other R² is selected from the group of H—, C₁₋₆-alkyl and oxo, and in cases wherein R² is attached to a nitrogen which is a ring member of Hc, this R² shall be independently of any other R² selected from: H—, C₁₋₆-alkyl-CO—, C₁₋₆-alkyl-O—CO—, C₁₋₆-alkyl-, phenyl-CO—, phenyl-O—CO— and (C₁₋₆-alkyl)₂N—CO—,
where the above-mentioned R² members may optionally be substituted independently of one another by one or more fluorine substituents;
R³ is selected from the group of
H—, hydroxy and C₁₋₆-alkyl-O—, whereby C₁₋₆-alkyl-O— may optionally be substituted by one or more fluorine, chlorine, bromine and HO—;
R⁴ and R⁵ independently of one another are selected from the group of H—, fluorine, and methyl;
x is 0, 1, 2, 3 or 4;
y is 0 or 1;
or a pharmaceutically acceptable salt, a tautomer or a stereoisomer thereof.
   Embodiment 40. The method of any one of embodiments 1-12, wherein the PDE9 inhibitor is represented by a compound of formula (XXII),

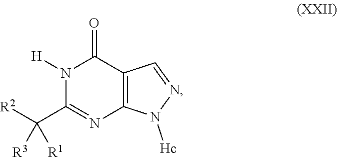

wherein
He is tetrahydropyranyl,
wherein one or more carbon ring atom(s) thereof optionally may be substituted by one or by two substituents independently selected from the group of fluorine, NC—, F₃C—, HF₂C—, FH₂C—, F₃C—CH₂—, C₁₋₆-alkyl-, C₁₋₆-alkyl-O— and up to one carbon ring atom may be substituted with oxo;
R¹ is the group, V-W-*
wherein
   W is phenyl;
   V is selected from the group of phenyl or heteroaryl, said heteroaryl being selected from the group of oxadiazolyl, triazolyl, pyrazolyl, pyrrolyl, furanyl, pyridyl, pyrimidyl and pyridazinyl;
   -* is the binding point by which W is attached to the CR²R³ group in formula (I); wherein W and V independently of each other optionally may be substituted by one or more substituents selected from the group of fluorine, chlorine, bromine, C₁₋₆-alkyl-, F₃C—, HF₂C—, FH₂C—, F₃C—CH₂—, F₃C—O—, HF₂C—O—, C₃₋₇-heterocycloalkyl-, H—O—C₁₋₆-alkyl-, C₁₋₆-alkyl-O—C₁₋₆-alkyl-, C₃₋₇-cycloalkyl-O—C₁₋₆-alkyl-, C₃₋₇-cycloalkyl-C₁₋₃-alkyl-O—C₁₋₆-alkyl-, phenyl-O—C₁₋₆-alkyl-, benzyl-O—C₁₋₆-alkyl-, H—O—, C₁₋₆-alkyl-O—, C₃₋₇-cycloalkyl-O—, C₃₋₇-cycloalkyl-C₁₋₃-alkyl-O—, phenyl-O—, benzyl-O—, N-morpholinyl, and NC—;
R² is selected from the group of H—, fluorine, F₃C—, HF₂C—, FH₂C—, and C₁₋₃-alkyl;
R³ is selected from the group of H—, fluorine, F₃C—, HF₂C—, FH₂C—, and C₁₋₃-alkyl.
   Embodiment 41. The method of any one of embodiments 1-12, wherein the PDE9 inhibitor is represented by a compound of formula (XXIII),

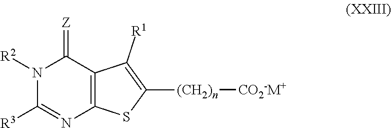

or stereoisomers, tautomers or hydrates thereof,
wherein:
R¹ is hydrogen, (C₁-C₆)alkyl, (C₁-C₆)alkoxy or (C₁-C₆) haloalkyl containing 1-6 halogen atoms;
R² is hydrogen, (C₁-C₆)alkyl, [(C₁-C₆)alkylene]aryl or amino;
R³ is hydrogen, (C₁-C₆)alkyl, (C₁-C₆)alkenyl, [(C₁-C₆)alkylene]N(R⁴)(R⁵), [(C₁-C₆)alkylene]S(R⁴) or —X—Y, or R² and R³ together with the atoms to which they are attached form a heterocyclyl;
   R⁴ and R⁵ are independently H or (C₁-C₆)alkyl;
X is a chemical bond, —CH₂— —CH(OH)—, —CH(C₆H₅)—, —CO— —CH₂CH₂— CH₂CO—, —COCH2-,
S, O or NH;
Y is cycloalkyl, heterocyclyl, aryl or heteroaryl;
Z is S or O;
n is 0, 1, 2, 3 or 4;
M⁺ is

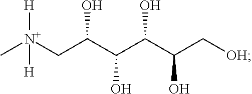

and
wherein any alkyl, alkylene, cycloalkyl, heterocyclyl, heteroaryl or aryl is optionally substituted with 1, 2 or 3 groups selected from OH, CN, halogen, (C₁-C₆)alkyl, O(C₁-C₆)alkyl, (C₂-C₆)alkenyl, haloalkyl, amino, oxo and nitro.
   Embodiment 42. The method of any one of embodiments 1-12, wherein the PDE9 inhibitor is represented by a compound of formula (XXIV),

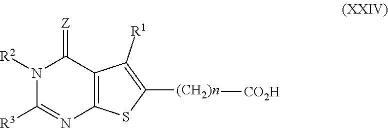

wherein:
R¹ is hydrogen, C₁₋₆ alkyl, C₁₋₆ alkoxyC₁₋₆ alkyl or C₁₋₆ haloalkyl containing 1-6 halogen atoms,
R² is hydrogen, C₁₋₆ alkyl, phenylC₁₋₆ alkyl or amino,
R³ is C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X group, or R² and R³ may together form tetramethylene,
X is a chemical bond, or CH₂, CH(OH), CH(C₆H₅), CO, CH₂CH₂, CH₂CO, COCH₂, S, O or NH and
Y is an aromatic carbocyclic group, benzyl, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from halogen atom, C₁₋₆ alkyl, C₁₋₆ haloalkyl containing 1-6 halogen atoms, C₁₋₆ haloalkyloxy containing 1-6 halogen atoms, C₁₋₆ haloalkylthio containing 1-6 halogen atoms, C₁₋₆ alkoxy, C₁₋₆ alkylthio, C₁₋₄ alkylenedioxy, carboxyl, C₁₋₆ alkoxycarbonyl, oxo, amino, nitro and phenyl,
Z is S or O, and n is 0 or an integer of 1-4; and pharmaceutically acceptable salts thereof.
   Embodiment 43. The method of any one of embodiments 1-12, wherein the PDE9 inhibitor is represented by a compound of formula (XXV),

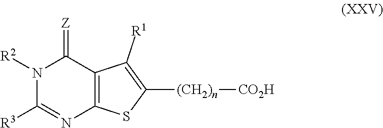

in which
   R¹ is hydrogen, C₁₋₆ alkyl, C₁₋₆ alkoxyC₁₋₆ alkyl or C₁₋₆ haloalkyl containing 1-6 halogen atoms,
   R² is hydrogen, C₁₋₆ alkyl, phenylC₁₋₆ alkyl or amino,
   R³ is C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X— group, or
   R² and R³ may together form tetramethylene,
   X is a direct bond, or CH₂, CH(OH), CH(C₆H₅), CO, CH₂CH₂, CH₂CO, COCH₂, S, O or NH and
   Y is an aromatic carbocyclic group, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from the group consisting of halogen atom, C₁₋₆ alkyl, C₁₋₆ haloalkyl containing 1-6 halogen atoms, C₁₋₆ haloalkyloxy containing 1-6 halogen atoms, C₁₋₆haloalkylthio containing 1-6 halogen atoms, C₁₋₆ alkoxy, C₁₋₆ alkylthio, C₁₋₄ alkylenedioxy, carboxyl, C₁₋₆ alkoxycarbonyl, oxo, amino, nitro and phenyl,
   Z is S or O, and
   n is 0 or an integer of 1-4, with the proviso that a case wherein R¹ is methyl, R² is hydrogen, R³ is benzyl, Z is O and n is 0 is excluded,
or a salt thereof.
   Embodiment 44. The method of embodiment 43, wherein the PDE9 inhibitor is a compound having a structure of

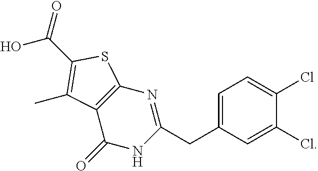 [SMILES: Cc1c(C(=O)O)sc2nc(Cc3ccc(Cl)c(Cl)c3)[nH]c(=O)c12]

Embodiment 45. The method of any one of embodiments 1-12, wherein the PDE9 inhibitor is represented by a compound of formula (XXVI),

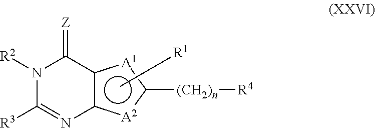

in which
R¹ stands for hydrogen, C₁₋₆ alkyl, C₁₋₆ alkoxy C₁₋₆ alkyl or C₁₋₆ haloalkyl containing 1-6 halogen atoms,
R² stands for hydrogen, C₁₋₆ alkyl, phenyl C₁₋₆ alkyl or amino,
R³ stands for C₁₋₆ alkyl, C₂₋₆ alkenyl, carbamoyl C₁₋₆ alkyl, amino C₁₋₆ alkyl, C₁₋₆ alkylamino C₁₋₆ alkyl, di-(C₁₋₆ alkyl)aminoC₁₋₆ alkyl, C₁₋₆ alkylthio or Y—X— group, or
R² and R³ may together form tetramethylene,
R⁴ stands for carboxylic acid or its equivalent,
X standing for a direct bond, or CH₂, CH(OH), CH(C₆H₅), CO, CH₂CH₂, CH₂CO, COCH₂, S, O or NH,
Y standing for aromatic carbocyclic group, aromatic heterocyclic group, 4-7-membered cycloalkyl group, 4-7-membered cycloalkenyl group, 5-7-membered saturated heterocyclic group containing 1 or 2 nitrogen atoms, or 5-7-membered saturated heterocyclic group forming a condensed ring with 5 or 6-membered saturated cyclic group and containing 1 or 2 nitrogen atoms, all of these groups optionally containing 1-3 substituents selected from halogen atom,
C₁₋₆ alkyl, C₁₋₆ haloalkyl containing 1-6 halogen atoms, C₁₋₆ haloalkyloxy containing 1-6 halogen atoms, C₁₋₆ haloalkylthio containing 1-6 halogen atoms, C₁₋₆ alkoxy, C₁₋₆ alkylthio, C₁₋₄alkylenedioxy, carboxyl, C₁₋₆ alkoxycarbonyl, oxo, amino, nitro and phenyl,
Z stands for S or O,
n is 0 or an integer of 1-4, and
one of A¹ and A² stands for carbon atom and the other stands for sulfur atom,
R¹ is attached to the carbon atom represented by either A¹ or A²,
with the proviso that there is excluded a case wherein A¹ is carbon atom, A² is sulfur atom and R⁴ is hydroxy 1,
or salts of the compounds are provided.
   Embodiment 46. The method of any one of embodiments 1-12, wherein the PDE9 inhibitor is represented by a compound of the following formulae (XXVII)—(XXXII):
thienopyrimidine derivatives represented by Formula (XXVII),

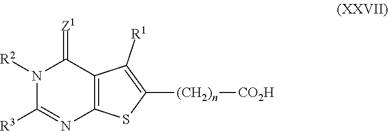

in the formula,
   R¹ stands for hydrogen, C₁₋₆ alkyl, C₁₋₆ alkoxyC₁₋₆ alkyl, or C₁₋₆ haloalkyl having 1-9 halogen atoms,
   R² stands for hydrogen, C₁₋₆ alkyl, phenylC₁₋₆ alkyl or amino,
   R³ either stands for C₂₋₆ alkyl, C₂₋₆ alkenyl, carbamoylC₁₋₆ alkyl, aminoC₁₋₆ alkyl, C₁₋₆ alkylaminoC₁₋₆ alkyl, di-(C₁₋₆ alkyl)aminoC₁₋₆ alkyl, C₁₋₆ alkylthio or Y—X—, or R² and R³ may together form tetramethylene,
   X stands for a direct bond or CH₂, CH(OH), CH(C₆H₅), CO, CH₂CH₂, CH₂CO, COCH₂, S, O, or NH,
   Y stands for an aromatic carbocyclic group, aromatic heterocyclic group, C₄₋₇ cycloalkyl, C₄₋₇ cycloalkenyl, 5- to 7-membered saturated heterocyclic group containing 1 or 2 nitrogen atoms, or 5- to 7-membered saturated heterocyclic group forming a condensed ring with a 5- to 6-membered saturated cyclic group and containing 1 or 2 nitrogen atoms, each of which may be optionally substituted with 1-3 substituents selected from halogen, C₁₋₆ alkyl, C₁₋₆ haloalkyl having 1-9 halogen atoms, C₁₋₆ alkoxy, C₁₋₆ haloalkoxy having 1-9 halogen atoms, C₁₋₆ alkylthio, C₁₋₆haloalkylthio having 1-9 halogen atoms,
   C₁₋₄ alkylenedioxy, carboxy, C₁₋₆ alkoxycarbonyl, oxo, amino, nitro and phenyl,
   Z¹ stands for S or 0, and
   n is 0 or an integer of 1-4,
or salts thereof;
   quinazoline derivatives represented by Formula (XXVIII),

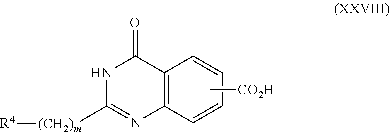

in the formula,
   R⁴ stands for phenyl or aromatic heterocyclic group which may be optionally substituted with 1-3 substituents selected from halogen, C₁₋₆ alkyl, C₁₋₆ haloalkyl having 1-9 halogen atoms and C₁₋₆ alkoxy, and
   m is an integer of 1-3,
or salts thereof;
   quinoxaline derivatives represented by Formula (XXIX),

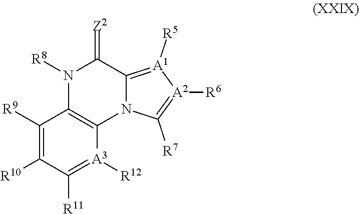

in the formula,
   R⁵ and R⁶ each independently stands for hydrogen; halogen; C₁₋₆ alkyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group; acyl which is optionally substituted with hydroxy, C₁₋₆ alkyl, C₂₋₆ alkenyl, C₂₋₆ alkynyl, C₁₋₆ haloalkyl having 1-9 halogen atoms, C₁₋₆ alkoxy, C₁₋₆ haloalkoxy having 1-9 halogen atoms, amino, carbocyclic group or heterocyclic group; amino which is optionally substituted with 1-2 substituents selected from C₁₋₆ alkyl, C₂₋₆ alkenyl, C₂₋₆ alkynyl, alkanoyl, carbocyclic group and heterocyclic group; hydroxy; or pyrimidinyl which is optionally substituted with halogen, C₁₋₆ alkyl, C₁₋₆ alkoxy, C₁₋₆ haloalkoxy having 1-9 halogen atoms, nitro or amino,
   R⁷ stands for C₁₋₉ alkyl, C₂₋₉ alkenyl or C₂₋₉ alkynyl which are optionally substituted with hydroxy, halogen, C₁₋₆ alkoxy, C₁₋₆ haloalkoxy having 1-9 halogen atoms, carboxy,
   C₁₋₆ alkoxycarbonyl, alkanoyl, amino (here the amino group may further be substituted with 1-2 substituents selected from C₁₋₆ alkyl, C₂₋₆ alkenyl, C₂₋₆ alkynyl, C₁₋₆haloalkyl having 1-9 halogen atoms, alkanoyl, carbocyclic group and heterocyclic group), amido, carbamoyl, oxo, carbocyclic or heterocyclic group (here the carbocyclic group and heterocyclic group each may further be substituted with hydroxy, halogen, C₁₋₆ alkyl,
   C₂₋₆ alkenyl, C₂₋₆ alkynyl, C₁₋₆ alkoxy, carboxy, C₁₋₆ alkoxycarbonyl, amino, amido or carbamoyl); aryl, saturated carbocyclic group or saturated heterocyclic group, each of which is optionally substituted with halogen, hydroxy, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with halogen, hydroxy, C₁₋₆ alkoxy, C₁₋₆ haloalkoxy having 1-9 halogen atoms, carboxy, C₁₋₆ alkoxycarbonyl, alkanoyl, amino, amido, carbamoyl, carbocyclic group or heterocyclic group, independently of each other), C₁₋₆haloalkoxy having 1-9 halogen atoms, carboxy, C₁₋₆ alkoxycarbonyl, alkanoyl, amino, amido, carbamoyl, carbocyclic group or heterocyclic group; carboxy; C₁₋₆ alkoxycarbonyl (here the C₁₋₆ alkoxy moiety in the C₁₋₆ alkoxycarbonyl may further be substituted with hydroxy, halogen, C₁₋₆ alkoxy, C₁₋₆haloalkoxy having 1-9 halogen atoms, carboxy,
   C₁₋₆ alkoxycarbonyl, amino, amido, carbamoyl, carbocyclic group or heterocyclic group); amido (here the amino moiety in the amido may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group); or carbamoyl (here the amino moiety in the carbamoyl may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), R⁸ stands for hydrogen; hydroxy; C₁₋₆ alkyl which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; or amino which is optionally substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group,
   R⁹ and R¹² each independently stands for hydrogen; halogen; C₁₋₆ alkyl, C₂₋₆ alkenyl or
   C₁₋₆ alkoxy each of which is optionally substituted with hydroxy, halogen, C₁₋₆ alkoxy,
   C₁₋₆haloalkoxy having 1-9 halogen atoms, carboxy, C₁₋₆ alkoxycarbonyl, alkanoyl, amino, amido, carbamoyl or oxo; cyano; or nitro,
   R¹⁰ and R¹¹ each independently stands for hydrogen; halogen; C₁₋₆ alkyl, C₂₋₆ alkenyl, C₂₋₆ alkynyl or C₁₋₆ alkoxy each of which is optionally substituted with hydroxy, halogen,
   C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); cyano; amino which is optionally substituted with 1-2 substituents selected from C₁₋₆ alkyl, C₂₋₆ alkenyl,
   C₂₋₆ alkynyl (here the C₁₋₆ alkyl, C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group and heterocyclic group), alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); carbocyclic group or heterocyclic group each of which is optionally substituted with hydroxy, halogen, C₁₋₆ alkyl, C₂₋₆ alkenyl, C₂₋₆ alkynyl, C₁₋₆ alkoxy (here the C₁₋₆ alkyl, C₂-6 alkenyl, C₂₋₆ alkynyl and C₁₋₆ alkoxy may further be substituted with, independently of each other, hydroxy, halogen, C₁₋₆ alkoxy, C₁₋₆haloalkoxy having 1-9 halogen atoms, carboxy,
   C₁₋₆ alkoxycarbonyl, C₁₋₆ alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, amino, amido or carbamoyl; COR¹³; or SO₂R¹³,
   R¹³ stands for hydrogen; hydroxy; C₁₋₆ alkyl, C₂₋₆ alkenyl, C₂₋₆ alkynyl or C₁₋₆ alkoxy, each of which is optionally substituted with hydroxy, halogen, C₁₋₆ alkoxy, C₁₋₆haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy,
   C₁₋₆ alkoxycarbonyl, amino, amido or carbamoyl); amino which may be substituted with 1-2 substituents selected from C₁₋₆ alkyl, C₂₋₆ alkenyl, C₂₋₆ alkynyl, (here the C₁₋₆ alkyl,
   C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy,
   C₁₋₆ alkoxycarbonyl, alkanoyl, amino, amido, carbamoyl, oxo, carbocyclic group or heterocyclic group), C₁₋₆haloalkyl having 1-9 halogen atoms, alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl,
   C₁₋₆ alkoxy, carboxy, C₁₋₆ alkoxycarbonyl, amino, amido or carbamoyl); or aziridin-1-yl, azetidin-1-yl, pyrrolidin-1-yl, piperidin-1-yl, piperazin-1-yl, morpholin-1-yl or pyrazol-1-yl, each of which may be substituted with 1-2 substituents selected from hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆haloalkoxy having 1-9 halogen atoms, carboxy,
   C₁₋₆ alkoxycarbonyl, alkanoyl, amino, amido, carbamoyl, oxo, carbocyclic group or heterocyclic group), C₁₋₆ haloalkoxy having 1-9 halogen atoms, carboxy,
   C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group and heterocyclic group (here the carbocyclic group and heterocyclic group each may further be substituted with hydroxy, halogen, C₁₋₆ alkyl,
   C₂₋₆ alkenyl, C₂₋₆ alkynyl, C₁₋₆ alkoxy, carboxy, C₁₋₆ alkoxycarbonyl, amino, amido or carbamoyl),
   Z² stands for S or O,
   A¹, A² and A³ stand for N or C, independently of each other, with the proviso that R⁵,
   R⁶ and R¹² are respectively absent where A¹, A² and A³ respectively stand for N,
or salts thereof;
   pyrazolopyrimidine derivatives represented by Formula (XXX),

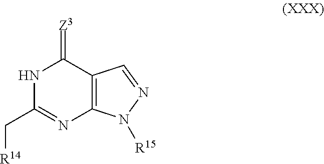

in the formula,
   R¹⁴ stands for phenyl which is substituted with 1-5 substituents selected from halogen, C₁₋₆ alkyl, trifluoromethyl, trifluoromethoxy, cyano, hydroxy, nitro and C₁₋₆ alkoxy,
   R¹⁵ stands for pentan-3-yl or C₄₋₆ cycloalkyl, and
   Z³ stands for S or O,
or salts thereof; and
   pyrazolopyrimidine derivatives represented by Formula (XXXI)

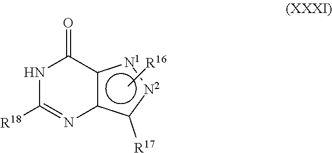

in the formula,
   R¹⁶ stands for hydrogen or C₁₋₆ alkyl, here the R¹⁶ binding to N¹ or N²,
   R¹⁷ stands for C₁₋₆ alkyl which is optionally substituted with hydroxy or alkoxy; C₃₋₇ cycloalkyl which is optionally substituted with alkyl, hydroxy or alkoxy; saturated 5- to 6-membered heterocyclic ring which is optionally substituted with alkyl, hydroxy or alkoxy; het¹; or Ar¹,
   R¹⁸ stands for C₁₋₆ alkyl which is optionally substituted with 1-2 substituents selected from optionally Ar²-substituted or C₁₋₆ alkyl-substituted C₃₋₇ cycloalkyl, OAr², SAr², NHC(O)C₁₋₆ alkyl, het², xanthine and naphthalene,
   here Ar¹ and Ar² standing for the group represented by the following formula (XXXII), independently of each other,

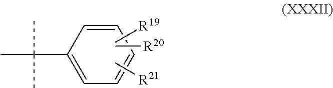

wherein R¹⁹, R²⁰ and R²¹ are either selected from hydrogen, halogen, phenoxy, phenyl, CF₃, OCF₃, R²², SR²² and OR²² (here R²² standing for het³ or C₁₋₆ alkyl which is optionally substituted with phenyl, which phenyl being optionally further substituted with 1-3 substituents selected from halogen, CF₃, OCF₃, C₁₋₆ alkyl and C₁₋₆ alkoxy), or R¹⁹ and R²⁰ together forming 3- or 4-atomic linker optionally containing 1-2 hetero atoms selected from O, S and N,
here het¹, het² and het³ may be the same or different, standing for aromatic 5- to 6-membered heterocyclic ring containing 1-3 hetero atoms selected from O, S and N, the heterocyclic ring being optionally substituted with 1-3 substituents selected from C₁₋₆ alkyl, C₁₋₆ alkoxy, halogen, and phenyl which may further be substituted with 1-3 substituents selected from halogen and C₁₋₆ alkyl, or salts thereof.
   Embodiment 47. The method of any one of embodiments 1-12, wherein the PDE9 inhibitor is represented by a compound of formula (XXXIII),

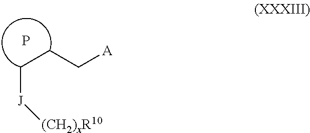

a stereoisomer or prodrug thereof, or a pharmaceutically acceptable salt of said compound, stereoisomer, or prodrug, wherein: A is

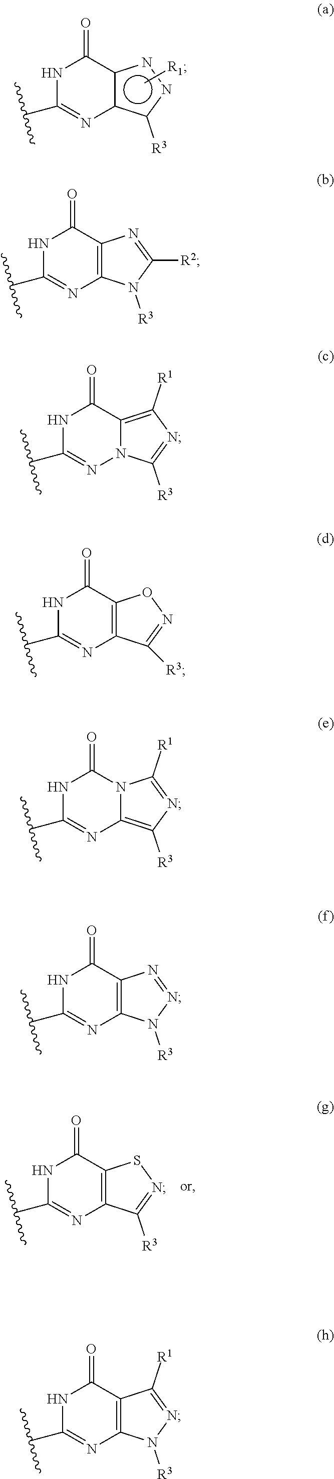

P, including the carbon atoms to which it is attached, is (C₃-C₈)cycloalkyl, (C₃-C₅)heterocycloalkyl, aryl, or heteroaryl; optionally and independently substituted with from 1 to 3 substituents independently selected from halogen, (C₁-C₅)alkyl, (C C₅)alkoxy, and trifluoromethyl;
J is O, S, —N(R¹⁵)—, —N(R¹⁵)CO—, —CON(R¹⁵)—, —SO₂ N(R¹⁵)—, or —N(R¹⁵) SO₂—; x is 0, 1, 2, 3, 4, 5, or 6;
R¹⁰ is —CO₂H, —CONR³OR³¹, —NR³⁰R³¹, or —N(R¹⁵)SO₂R⁴⁰;
R and R² are independently H or (CrC₃)alkyl; R³ is (CrC₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C C₅)alkyl, and (C C₅)alkoxy;
R⁵ is H or (C₁-C₅)alkyl;
R³⁰ and R³¹ are taken separately and are independently H, (C_{ι}-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C C₅)alkyl, —CO₂R⁴⁰, —COR⁴⁰, —OR⁴⁰, —CONR⁵⁰R⁵¹, —NR⁵⁰R⁵¹, and —SO₂R⁴⁰;
or
R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C C₅)alkyl,—CO₂R⁴⁰, —COR⁴⁰, —OR⁴⁰, —CONR⁵⁰R⁵¹, —NR⁵⁰R⁵¹, and —SO₂R⁴⁰;
R⁴⁰ is H, (C C₅)alkyl, (C₃-C₈)cycloalkyl, (C₃-C₈)heterocycloalkyl, aryl, or heteroaryl;
R⁵⁰ and R⁵¹ are taken separately and are independently H, (C_{ι}-C₅)alkyl, (C₃-C₈)cycloalkyl, (C₃-C₈)heterocycloalkyl, aryl, or heteroaryl; or
R⁵⁰ and R⁵¹ are taken together with the nitrogen atom to which they are attached to form a 5- to 8-membered heterocycloalkyl ring, said ring optionally having 1 additional heteroatom independently selected from N, O, and S.
   Embodiment 48. The method of any one of embodiments 1-12, wherein the PDE9 inhibitor is represented by a compound of formula (XXXIV) or a salt thereof,

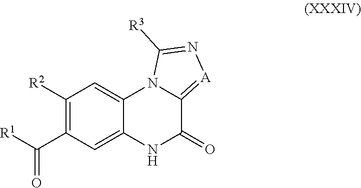

wherein
A is N or CH,
R¹ is 2,3-dihydroindolyl, 1,3-dihydroisoindolyl or dihydropyrrolopyridyl, each of which may be substituted,
R² is halogen, or lower alkyl, —O-lower alkyl or cycloalkyl, each of which may be substituted,
R³ is lower alkyl, cycloalkyl or a saturated heteroring, each of which may be substituted.
   Embodiment 49. The method of any one of embodiments 1-12, wherein the PDE9 inhibitor is represented by a compound of formula (XXXV) or a pharmaceutically acceptable salt, solvate or prodrug thereof,

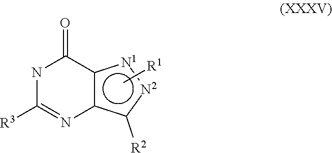

wherein
R¹ is H or C₁₋₆ alkyl, wherein R¹ is attached to either N¹ or N²;
R² is C₁₋₆ alkyl optionally substituted by hydroxy or alkoxy; C₃₋₇ cycloalkyl optionally substituted by alkyl, hydroxy or alkoxy; a saturated 5-6-membered heterocycle (preferably tetrahydrofuran, tetrahydrothiophene, pyrrolidine or piperidine) optionally substituted by alkyl, hydroxy or alkoxy; het¹ or Ar¹;
R³ is C₁₋₆ alkyl optionally substituted by 1 or 2 groups independently selected from: Ar²; C₃₋₇cycloalkyl optionally substituted by C₁₋₆alkyl; OAr²; SAr²; NHC(O)C₁₋₆ alkyl; het²; xanthene; and naphthalene;
wherein Ar¹ and Ar² are independently groups of formula

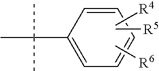

wherein R₄, R₅ and R₆ are independently selected from: hydrogen, halo, phenoxy, phenyl, CF₃, OCF₃, R⁷, SR⁷ and OR⁷, wherein R⁷ is C₁₋₆ alkyl optionally substituted by het³ or by a phenyl group optionally substituted by 1, 2 or 3 groups independently selected from halo, CF₃, OCF₃, C₁₋₆ alkyl and C₁₋₆ alkoxy; or wherein R⁴ and R⁵ combine to form a 3 or 4 atom link, wherein said link may incorporate one or two heteroatoms independently selected from O, S and N; and
wherein het¹, het² and het³, which may be the same or different, are aromatic 5-6 membered heterocycles containing 1, 2 or 3 heteroatoms, independently selected from O, S and N, said heterocycle optionally substituted by 1, 2 or 3 substituents, independently selected from C₁₋₆ alkyl, C₁₋₆alkoxy, halo and phenyl optionally substituted by 1, 2 or 3 groups independently selected from halo and C₁₋₆ alkyl;
with the provisos that when
   a) R¹ is attached to N¹, R¹ is C₁₋₃ alkyl and R² is propyl then R³ is not methyl substituted by Ar¹, and
   b) R¹ is attached to N¹, R¹ is C₁₋₆ alkyl and R² is methyl then R³ is not C₁₋₄alkyl substituted by Ar¹.
      Embodiment 50. The method of any one of embodiments 1-12, wherein the PDE9 inhibitor is represented by a compound of formula (XXXVI)

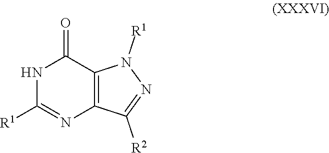

or a pharmaceutically acceptable salt, solvate or prodrug thereof, wherein:
R¹ is H or (C₁-C₆)alkyl;
R² is (C₁-C₆)alkyl, straight chain or branched chain, (C₃-C₇)cycloalkyl or heteroaryl; R³ is (C₁-C₆)alkyl, straight chain or branched chain, optionally substituted by 1-2 groups each independently selected from Ar, (C₃-C₇)cycloalkyl, OAr, SAr, NC(O)(C₁-C₆)alkyl, heteroaryl, xanthene, and naphthalene;
Ar is a group of formula

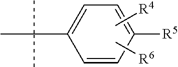

wherein R⁴, R⁵ and R⁶ are each independently selected from H, halo, phenoxy, phenyl, CF₃, OCF₃, S(C₁-C₆)alkyl, (C₁-C₆)alkyl, O(C₁-C₆)alkyl, said alkyl optionally substituted by a heteroaryl group or by a phenyl group, wherein said phenyl group is optionally substituted by 1-3 groups selected from halo, CF₃, OCF₃ and (C₁-C₆)alkyl; or wherein R⁴ and R⁵ may combine to form a (C₂-C₃)alkyl link, wherein said link may optionally incorporate a heteroatom selected from O, S and N; and heteroaryl is aromatic 5-6 membered heterocycle containing 1-3 heteroatoms, each independently selected from O, S and N, said heterocycle optionally substituted by 1-3 substituents, each independently selected from (C₁-C₆)alkyl, halo and phenyl, said phenyl optionally substituted by 1-3 groups selected from halo and (C₁-C₆)alkyl; with the proviso that when R¹ is —CH₃, R² cannot be —CH₂CH₂CH₃.
   Embodiment 51. The method of any one of embodiments 1-12, wherein the PDE9 inhibitor is represented by a compound of formula (XXXVII),

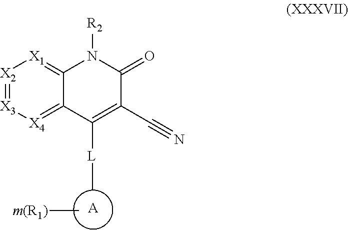

wherein, X₁, X₂, X₃, and X₄ are each independently CR₃ or N, and X₁, X₂, X₃, and X₄ are not simultaneously CR₃;
R³, at each occurrence, is independently selected from the group consisting of hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryloxy, wherein C₁₋₆ alkyl, C₁₋₆ alkoxy, C₁₋₆ alkylamino, (C₁₋₆ alkyl)₂ amino, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, C₁₋₆ alkylcarbonyl, aminocarbonyl, C₁₋₆ alkylaminocarbonyl, (C₁₋₆ alkyl)₂ aminocarbonyl, 4-6 membered heterocyclylcarbonyl and 5-6 membered heteroaryl-oxy are unsubstituted or optionally substituted with one or more groups independently selected from the group consisting of hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyloxy, C₃₋₆ cycloalkyl, C₂₋₈ alkynyl, halogenated C₁₋₆ alkyl, C₂₋₈ alkenyl, halogenated C₁₋₆ alkoxy, 4-6 membered heterocyclyl which is unsubstituted or optionally substituted with a substituent, and heteroaryl which is unsubstituted or optionally substituted with a substituent;
the substituent in the above 4-6 membered heterocyclyl optionally substituted with a substituent and heteroaryl optionally substituted with a substituent is selected from the group consisting of hydroxy, amino, carboxyl, cyano, nitro, halogen, C₁₋₆ alkyl and C₁₋₆ alkoxy;
L is a bond, —NH—(CH₂)ₜ—, and t is 0, 1, 2 or 3;
ring A is selected from the group consisting of 3-12 membered heterocyclyl, aryl, 5-10 membered heteroaryl, 3-12 membered cycloalkyl, and 3-12 membered cycloalkenyl, wherein 3-12 membered heterocyclyl has an hetero atom selected from one of O, S, N or any combination thereof, and the S atom may be optionally oxidized to S(O) or S(O)₂, and the 5-10 membered heteroaryl has an hetero atom selected from one of O, S, N or any combination thereof;
each R₁ is independently selected from the group consisting of hydrogen, hydroxy, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected form the group consisting of hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, and C₁₋₆ alkylsulfonylamino;
m is 0, 1, 2 or 3; and
R₂ is selected from the group consisting of hydrogen, C₁₋₆ alkyl, C₂₋₈ alkenyl, C₂₋₈ alkynyl, and halogenated C₁₋₆ alkyl.
   Embodiment 52. The method of any one of embodiments 1-12, wherein the PDE9 inhibitor is represented by a compound of formula (XXXVIII) or its pharmaceutically acceptable salts, isomers, deuterated compounds, metabolites and prodrugs:

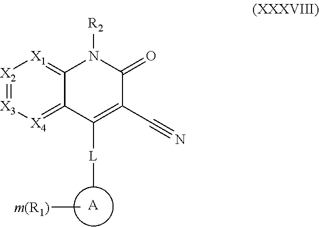

wherein, X₁, X₂, X₄ are independently selected from CR′ or N, X_{3 is} selected from CR₃ or N, and X₁, X₂, X₃, X₄ are at least one of the N heteroatoms can be optionally oxidized to

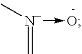

R′ and R₃ are independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁-6 alkylthio, C₃₋₆ cycloalkyl, 4-6 membered heterocyclic group, 5-6 membered heteroaryl, aryl, C₁₋₆ alkane Carbonyl, aminocarbonyl, C₁₋₆ alkylaminocarbonyl, (C₁₋₆ alkyl)₂ aminocarbonyl, 4-6 membered heterocyclic carbonyl and 5-6 membered heteroaryl-oxy, wherein said C₁₋₆ alkyl, C₁₋₆ alkoxy, C₁₋₆ alkylamino, (C₁₋₆ alkyl)₂ amino, halogenated C₁₋₆ alkoxy, C₂₋₈ alkenyl, C₂₋₈ alkynyl, C₁₋₆ alkylsulfonyl, C₁₋₆ alkylthio, C₃-6 cycloalkyl, 4-6 membered heterocyclic group, 5-6 membered heteroaryl, aryl, C₁₋₆ alkylcarbonyl, aminocarbonyl, C₁-6 alkylaminocarbonyl, (C₁₋₆ alkyl)2 aminocarbonyl, 4-6 membered heterocyclic carbonyl and 5-6 membered heteroaryl-oxy Unsubstituted or optionally by one or more independently selected from hydroxyl, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkylcarbonyl Oxy, C₃₋₆ cycloalkyl, C₃₋₆ cycloalkylcarbonyloxy, C₂₋₈ alkynyl, halogenated C₁₋₆ alkyl, C₂₋₈ alkenyl, halogenated C₁₋₆ alkoxy,

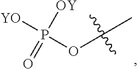

a 4-6 membered heterocyclic group that is unsubstituted or optionally substituted by one or more independent substituents, or a heteroaryl group that is unsubstituted or optionally substituted by one or more independent substituents;
the 4-6 membered heterocyclic group optionally substituted by one or more independent substituents, and the substituent of the heteroaryl group optionally substituted by one or more independent substituents are selected from hydroxyl, amino, carboxyl, cyano, nitro, halogen, C₁₋₆ alkyl and C₁₋₆ alkoxy;
Y is selected from metal ions or organic ammonium ions; preferably Na⁺, K⁺, NH₄⁺;
L is a bond, —NH—(CH₂)t-, t is 0, 1, 2 or 3;
Ring A is a 3-12 membered heterocyclic group, a 5-10 membered heteroaryl group, a 3-12 membered cycloalkyl group, a 3-12 membered cycloalkenyl group, wherein the heteroatom of the 3-12 membered heterocyclic group is selected from One of O, S, and N or any combination thereof, S atoms can be optionally oxidized to S(O) or S(O) 2, C atoms can be optionally oxidized to C(O), and N heteroatoms can be Is optionally oxidized to

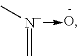

the heteroatom of the 5-10 membered heteroaryl group is selected from one of O, S, and N or any combination thereof;
each R₁ is independently selected from hydrogen, deuterium, hydroxyl, amino, carboxyl, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkyl Sulfonyl, C₁₋₆ alkylthio, 3-12 membered cycloalkyl, 3-12 membered cycloalkenyl, 3-12 membered heterocyclic group, aryl and 5-10 membered heteroaryl, wherein C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkane Oxy, C₂-8 alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally selected from hydroxyl, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C Substitution of 1-6 alkylsulfonylamino groups;
m is 0, 1, 2 or 3;
R₂ is selected from hydrogen, C₁₋₆ alkyl, C₂₋₈ alkenyl, C₂₋₈ alkynyl, halogenated C₁₋₆ alkyl; requirement is,
(1) when X₂ is N, X₁ is CH, X₃ is CR₃, and X₄ is CH, ring A is

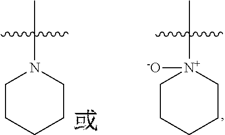

(i) when

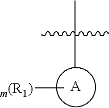

for

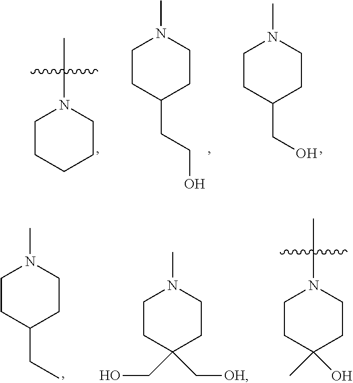

when, R₃ is not H;
(ii) when

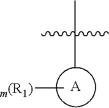

for

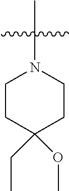 [SMILES: *N1CCC(CC)(OC)CC1]

when, R₃ is not Cl;
(iii) when

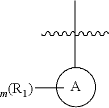

for

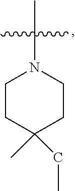 [SMILES: *N1CCC(C)(CC)CC1]

R₃ is not H,

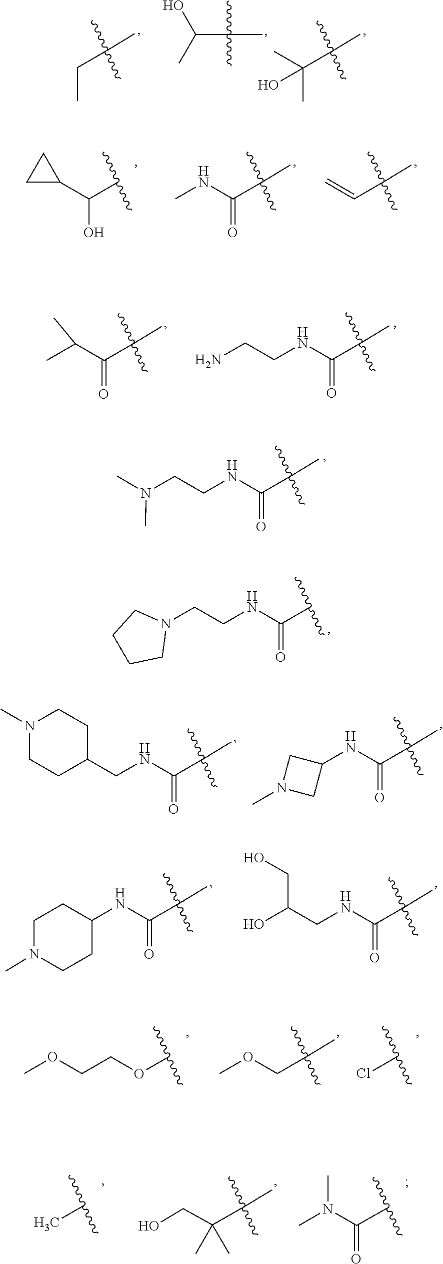

(2) when X₂ and X₄ are N, X₁ is CH, X₃ is CR₃, and ring A is

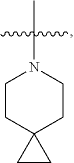 [SMILES: *N1CCC2(CC1)CC2]

when m is 0, R₃ is not a methylthio group;
(3) when X₄ is N, X₁ and X₂ are respectively CH, X₃ is CR₃, and ring A is

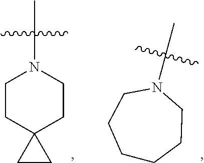

when m is 0, R₃ is not hydrogen;
(4) when X₁ is N, X₂ and X₄ are CR′, X₃ is CR₃, and ring A is pyrrolidinyl,

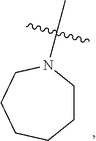 [SMILES: *N1CCCCCC1]

when m is 0, R₂ is not H or C₁₋₆ alkyl.
preferably, when X₂ is N, X₁ is CH, X₃ is CR₃, and X₄ is CH,

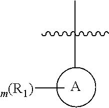

for

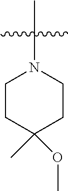 [SMILES: *N1CCC(C)(OC)CC1]

when R₃ is selected from isopropyl, cyclopropyl, hydroxymethyl,

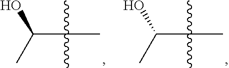

C₁₋₆ alkylcarbonyl, C₁₋₆ alkylcarbonyloxy C₁₋₆ alkyl, is

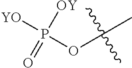

substituted C₁₋₆ alkyl, C₃₋₆ cycloalkylcarbonyloxy C₁₋₆ alkyl, deuterated C₁₋₆ alkyl; preferably, when X₂ is N, X₁ is CH, X₃ is CR₃, and X₄ is CH, ring A is

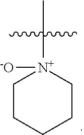

Embodiment 53. The method of any one of embodiments 1-12, wherein the PDE9 inhibitor is represented by a compound of formula (XXXIX) or a pharmaceutically acceptable salt or isomer thereof:

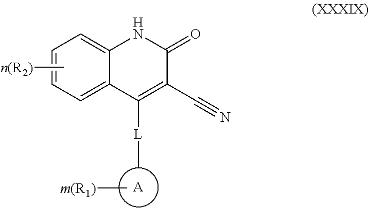

each R₂ is independently selected from hydrogen, hydroxy, amino, carboxy, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclic group, C₁₋₆ alkylcarbonyl, aminocarbonyl, C₁₋₆ alkylaminocarbonyl, (C₁₋₆ alkyl Group) 2 aminocarbonyl, aryl, 5-6 membered heteroaryl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclic group, C₁₋₆ alkylcarbonyl, aminocarbonyl, C₁₋₆ alkylaminocarbonyl, (C₁₋₆ alkyl)₂ aminocarbonyl, aryl, 5-6 membered heteroaryl, 4-6 membered heterocyclylcarbonyl and 5-6 membered heteroaryl-oxygen The group is unsubstituted or optionally one or more independently selected from hydroxy, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino, C₁₋₆ alkylsulfonylamino, C₁₋₆ alkyl Carbonyloxy, C₃₋₆ cycloalkyl, C₂₋₈ alkynyl, halogenated C₁₋₆ alkyl, C₂₋₈ alkenyl, halogenated C₁₋₆ alkoxy, unsubstituted or any A group substituted by a 4-6 membered heterocyclic group substituted by a substituent, a heteroaryl group which is unsubstituted or optionally substituted by a substituent;
The substituents of the above-mentioned 4-6 membered heterocyclic group optionally substituted by substituents and heteroaryl groups optionally substituted by substituents are selected from the group consisting of hydroxy, amino, carboxy, cyano, nitro, halogen, and C₁₋₆ alkyl group and C₁₋₆ alkoxy;
L is a bond, —NH—(CH₂)ₜ—, t is 0, 1, 2 or 3;
Ring A is a 3-8 membered monoheterocyclic group, 6-12 membered bridged heterocyclic group, 6-12 membered spiro heterocyclic group, 6-12 membered heterocyclic group, aryl group, 5-10 membered heteroaryl group, 3-12 membered cycloalkyl, 3-12 membered cycloalkenyl, wherein the hetero atom of the heterocyclic group is selected from one of O, S, N or any combination thereof, S atom may be optionally oxidized to S (O) or S(O)₂, the C atom may be optionally oxidized to C(O), and the hetero atom of the 5-10 membered heteroaryl group is selected from one of O, S, N or any combination thereof; each R₁ is independently selected from hydrogen, hydroxy, amino, carboxy, cyano, nitro, halogen, C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂ amino, halogenated C₁₋₆ alkyl, halogenated C₁₋₆ alkoxy, C₂-8 alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclic group, aryl group and 5-10 membered heteroaryl group are unsubstituted or optionally selected from hydroxy, amino, carboxy, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂ amino, C₁₋₆ alkylcarbonylamino and C₁₋₆ Alkylsulfonylamino group substitution;
m and n are independently 0, 1, 2 or 3;
when ring A is a 3-8 membered monoheterocyclic group, R_{2 is} not hydrogen;
when ring A is phenyl, L is not a bond;
when ring A is

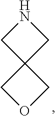

R₂ is not hydrogen.
   Embodiment 54. The method of any one of embodiments 1-12, wherein the PDE9 inhibitor is represented by a compound of formula (XXXX),

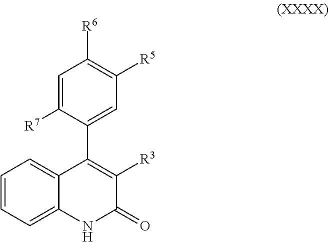

wherein:
   R³ is selected from the group consisting of:
      (1) —CN,
      (2) —CH₃, and
      (3) —CF₃;
   R⁵, R⁶ and R⁷ are independently selected from the group consisting of:
      (1) hydrogen,
      (2) C₁₋₆alkyl, and
      (3) fluoro,
      or R⁵ and R⁶ together with the carbons to which they are attached are joined together to form a fused phenyl ring;
   or a pharmaceutically acceptable salt thereof.
      Embodiment 55. The method of any one of embodiments 1-12, wherein the PDE9 inhibitor is represented by a compound of formula (XXXXI),

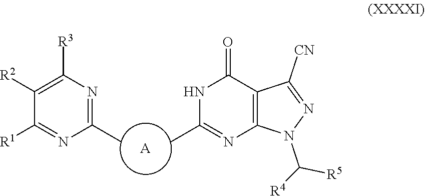

wherein:
   A is a cyclobutyl ring, which is unsubstituted or or substituted with substituents selected from: fluoro and methyl;
   R¹, R² and R³ are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl, and
      (7) —CN;
      or a pharmaceutically acceptable salt thereof.
         Embodiment 56. The method of any one of embodiments 1-12, wherein the PDE9 inhibitor is represented by a compound of formula (XXXXII),

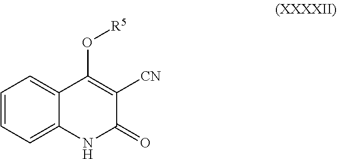

wherein:
R⁵ is selected from the group consisting of
   (1) —C₁₋₆alkyl, which is unsubstituted or substituted with hydroxy, oxetane, or allyl,
   (2) -tetrahydropyranyl, and
   (3) —C₆₋₇cycloalkyl;
   or a pharmaceutically acceptable salt thereof.
      Embodiment 57. The method of any one of embodiments 1-12, wherein the PDE9 inhibitor is represented by a compound of formula (XXXXIII),

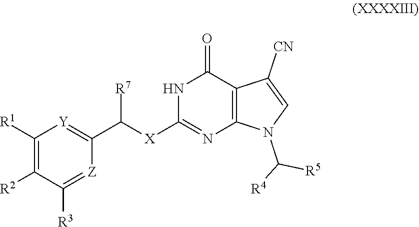

wherein:
   X is —N(R⁶)— or —C(R⁸R⁹)—.
   Y is —N═ or —CH═;
   Z is —N═ or —CH═;
   R¹, R² and R³ are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) azetidine, morpholine, piperidine, or pyrrolidine, and
      (8) —CN;
   R⁶ is selected from:
      (1) hydrogen, and
      (2) C₁₋₆alkyl;
   R⁷ is selected from:
      (1) hydrogen,
      (2) C₁₋₆alkyl, which is unsubstituted or substituted with substituents selected from: hydroxy and fluoro,
      (3) C₂₋₆alkenyl,
      (4) C₃₋₆cycloalkyl, which is unsubstituted or substituted with substituents selected from: hydroxy and fluoro, and
      (5) azetidine, morpholine, piperidine, or pyrrolidine;
      or R⁶ and R⁷ and the —CH—N— to which they are attached are joined to form an azetidine or a pyrrolidine ring;
   R⁸ and R⁹ are independently selected from:
      (1) hydrogen, and
      (2) C₁₋₆alkyl;
      or a pharmaceutically acceptable salt thereof.
         Embodiment 58. The method of any one of embodiments 1-12, wherein the PDE9 inhibitor is represented by a compound of formula (XXXXIV),

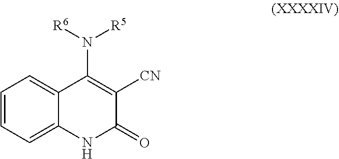

wherein:
R⁵ is selected from the group consisting of
   (1) hydrogen, and
   (2) —C₁₋₆alkyl;
   R⁶ is —C₁₋₆alkyl, which is unsubstituted or substituted with oxetane;
   or R⁵ and R⁶ are joined together with a —C₃₋₆alkyl through the nitrogen atom to which they are attached to form a azetidine, pyrrolidine, piperidine, or azepan ring, which is unsubstituted or substituted with pyrazole or one or two —C₁₋₆alkyl;
   or a pharmaceutically acceptable salt thereof.
      Embodiment 59. The method of any one of embodiments 1-12, wherein the PDE9 inhibitor is represented by a compound of formula (XXXXV),

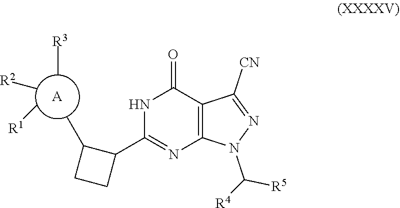

wherein: A is a pyrazinyl, pyrazolyl, pyridyl, pyridyl-N-oxide, 5-pyrimidinyl, pyridazinyl, pyrrolopyrazolyl, dihydropyrrolopyrazolyl, morpholinyl, oxomorpholinyl, or oxadiazolyl ring;
   R¹, R² and R³ as are present are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl,
      (7) C₂₋₆alkynyl, and
      (8) —CN;
   R⁴ is selected from:
      (1) hydrogen,
      (2) —CH₃;
      (3) —CF₃,
      (4) —CH₂OH,
      (5) —CO₂H, and
      (6) —CH₂CH₃;
   R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} as are present are independently selected from:
      (1) hydrogen,
      (2) halogen,
      (3) hydroxyl,
      (4) C₁₋₆alkyl, which is unsubstituted or substituted with substituents selected from: hydroxy and fluoro,
      (5) —O—C₁₋₆alkyl, which is unsubstituted or substituted with substituents selected from fluoro,
      (6) C₃₋₆cycloalkyl, and
      (7) —CN;
      or a pharmaceutically acceptable salt thereof.
         Embodiment 60. The method of any one of embodiments 1-12, wherein the PDE9 inhibitor is represented by a 1-benzyl-2, 5,6, 8-tetrahydro-3-oxo-2,7-naphthyridine-4-carbonitrile compound of formula (XXXXVI):

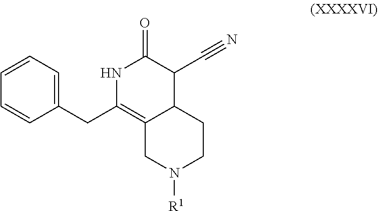

wherein R is —COCH₂R², COO—C₁₋₆-alkyl, CH₂CONR³R⁴, pyridinyl, pyrimidinyl or C₁₋₆-alkyl which is optionally substituted with C₁₋₆-alkoxy, wherein R² is morpholinyl or piperazinyl optionally substituted with C₁₋₆-alkyl,
R³ and R⁴ are independently selected from C₁₋₆-alkyl, and benzyl or R³ and R⁴ together with nitrogen to which they are attached form a morpholinyl or piperazinyl optionally substituted with C₁₋₆-alkyl, and pharmaceutically acceptable acid addition salts thereof.
   Embodiment 61. The method of any one of embodiments 1-12, wherein the PDE9 inhibitor is represented by a compound of formula (XXXXVII), or a pharmaceutically acceptable salt thereof:

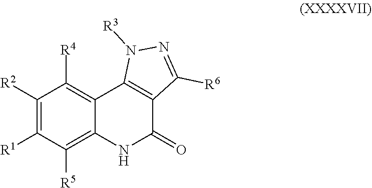

wherein
one of R¹ and R² is hydrogen, halogen, halogeno-lower alkyl, or lower alkyl, —O-lower alkyl or cycloalkyl, each of which may be substituted, and the other is a group of formula (II):

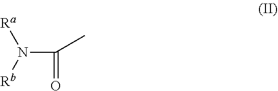

R³ is lower alkyl, cycloalkyl or a saturated hetero ring, each of which may be substituted,
R⁴, R⁵ and R⁶ are the same as or different from each other, and each is hydrogen or lower alkyl, and
R^{a} and R^{b} are the same as or different from each other, and each is hydrogen, or lower alkyl, cycloalkyl, aryl or a hetero ring, each of which may be substituted, or
R^{a} and R^{b} are combined with the adjacent nitrogen atom to form a monocyclic nitrogen-containing hetero ring or a polycyclic nitrogen-containing hetero ring, each of which may be substituted.
   Embodiment 62. The method of any one of embodiments 1-12, wherein the PDE9 inhibitor is represented by a compound of formula (XXXXVIII), or a pharmaceutically acceptable salt thereof:

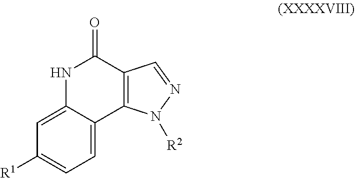

wherein R¹ is a group represented by the formula:

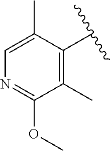 [SMILES: *c1c(C)cnc(OC)c1C]

a group represented by the formula:

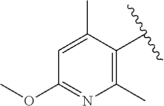

or a group represented by the formula:

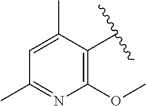

and R² is a 3-methyltetrahydro-2H-pyran-4-yl group or 4-methoxycyclohexyl group.
   Embodiment 63. The method of any one of embodiments 1-12, wherein PDE9 inhibitors are represented by a compound of formula (XXXXIX):

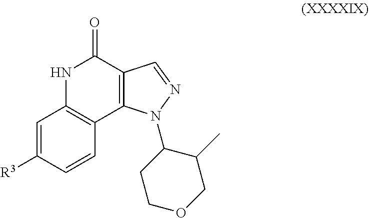

wherein R³ is a group represented by the formula:

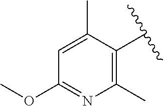

or a group represented by the formula:

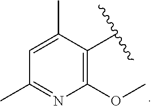

Embodiment 64. The method of any one of embodiments 1-12, wherein the PDE9 inhibitor is represented by a compound of formula (L),

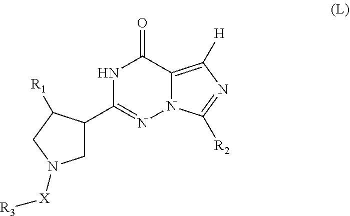

or a pharmaceutically acceptable salt thereof,
wherein:
   X is selected from a bond, C(O) or S(O)₂;
   R¹ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and heterocycloalkyloxy, each of which may be optionally substituted with one or more substituents selected from the group consisting of halogen (e.g., F or CI), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, and —S(O)₂N [(C₁-C₄)alkyl(C₁-C₄)alkyl];
   R² is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen (e.g., F or C₁), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl]; and
   R³ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen (e.g., F or C₁), —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl].
      Embodiment 65. The method of any one of embodiments 1-12, wherein the PDE9 inhibitor is represented by a compound of formula (LI),

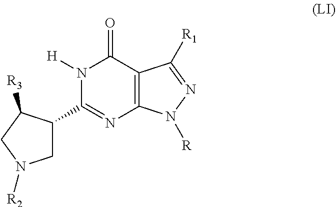

and pharmaceutically acceptable salts thereof,
wherein R is selected from the group consisting of (C₁-C₆)alkyl, (C₂-C₆)alkenyl, (C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl,
R₁ is selected from the group consisting of hydrogen, (C₁-C₄)alkyl, (C₂-C₄)alkenyl, (C₂-C₄)alkynyl, (C₁-C₄)haloalkyl, and cyclopropyl,
R² is selected from the group consisting of (C₁-C₆)alkyl, (C₂-C₆)alkenyl, (C₂-C₆)alkynyl, (C₁-C₆)haloalkyl, heteroaryl selected from the group consisting of pyridinyl, pyridazinyl, pyrimidinyl and pyrazinyl, and ER₅, wherein the heteroaryl optionally may be substituted with one to three substituents independently selected from the group consisting of (C₁-C₄)alkyl and (C₁-C₄)haloalkyl,
R³ is selected from the group consisting of hydrogen, (C₁-C₄)alkyl, (C₂-C₄)alkenyl, (C₂-C₄)alkynyl, (C₃-C₆)cycloalkyl, and (C₁-C₄)haloalkyl,
E is selected from the group consisting of —CH₂—, —CH₂CH₂—, —CH₂CH₂CH₂—, and —C(O)—, R₅ is selected from the group consisting of (C₃-Cₑ)cycloalkyl, heterocycloalkyl, aryl, aryloxy, and heteroaryl, any of which optionally may be substituted with one to three substituents, such substituents being independently selected from the group consisting of (C₁-C₄)alkyl, (C₂—C₄)alkenyl, (C₂-C₄)alkynyl, (C₁-C₄)hydroxyalkyl, (C₁-C₄)haloalkyl, (C₁-C₄)alkoxy, (C₁-C₄)haloalkoxy, (C₃-C₈)cycloalkyl, halo, cyano, phenyl, morpholinyl, (C₁-C₄)alkylamino, pyrazolyl, triazolyl, and imidazolyl.
Preferably, R is selected from the group consisting of ethyl, isopropyl, trifluoroethyl, cyclobutyl, cyclopentyl, difluorocyclohexyl, methoxyphenyl, and tetrahydro-2H-pyran-4-yl, R₁ is hydrogen or methyl, R₂ is methyl, trifluoroethyl, trifluorobutyl, pyrimidinyl, trifluoromethylpyπrmidinyl, Or ER₅, R³ is methyl, ethyl, isopropyl, trifluoromethyl, trifluoroethyl, or cyclopropyl, E is —CH₂— or —C(O)—, and R⁵ is selected from the group consisting of substituted or unsubstituted cyclopentyl, morpholinyl, phenyl, naphthyl, benzyloxy, pyrimidinyl, pyndinyl, quinolinyl, quinoxalinyl, pyrazinyl, pyrazolyl, benzimidazolyl, cinnolinyl, naphthydπnyl, pyπrdo[2,3-b]pyrazinyl, ιmιdazo[4,5-c]pyπdinyl, benzothiadiazolyl, tetrahydropyrazolo[1,5-a]pyrιdιnyl, dihydrobenzodioxinyl, imidazolyl, dihydrobenzofuranyl, triazolyl, oxazolyl, isoxazolyl, benzodioxinyl, thiazolyl, ιmιdazo[1,2-a]pyrιnyl, tetrahydrobenzothiazolyl, dihydrobenzoxazinyl, tetrahydropyranyl, tetrahydropyrazolo[1,5-a]azepinyl, and dιhydropyrrolo[1,2-b]pyrazolyl.
More preferably, R is selected from the group consisting of isopropyl, cyclobutyl, cyclopentyl, and tetrahydro-2H-pyranyl, R₁ is hydrogen, R₂ is ER₅, R₃ is methyl or ethyl, E is —CH₂—, and R₅ is selected from the group consisting of phenyl, pyrιmιdιn-2-yl pyridin-2-yl, pyrazιn-2-yl, and 5-methylρyrazιn-2-yl.
   Embodiment 66. The method of any one of embodiments 1-12, wherein the PDE9 inhibitor is represented by a compound of formula (LII),

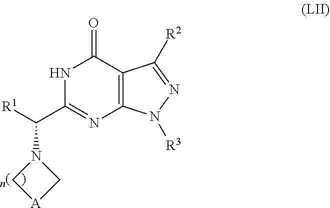

and pharmaceutically acceptable salts thereof, wherein:
R₁ is selected from the group consisting of (i) hydrogen, (ii) (C₁-C₄)alkyl, (iii) (C₂-C₄)alkenyl, (iv) (C₂-C₄)alkynyl, (v) (C₁-C₄)alkoxy, (vi) (C₁-C₄)haloalkyl, (vii) (C₃-C₆)cycloalkyl, optionally substituted with one to three substituents, the substituents being independently selected from the group consisting of (C₁-C₄)alkyl, (C₁-C₄)alkoxy, halo, (C₁—C₄)haloalkyl, (C₁-C₄)haloalkoxy, cyano, carboxy, and carbamoyl, (viii) 4 to 10 member heterocycloalkyl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl, (ix) aryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl, and (x) heteroaryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, cyano, carboxy, and carbamoyl;
R² is selected from the group consisting of hydrogen, (C₁-C₄)alkyl, (C₁-C₄)haloalkyl, cyano, and (C₃-C₆)cycloalkyl; R³ is selected from the group consisting of (C₁-C₆)alkyl, (C₂-C₆)alkenyl,
(C₂-C₆)alkynyl, (C₃-C₈)cycloalkyl, heterocycloalkyl, aryl, and heteroaryl, each of which optionally may be substituted with one to three substituents, the substituents being independently selected from the group consisting of (C₁-C₄)alkyl, (C₁-C₄)alkoxy, halo, and (C₁-C₄)haloalkyl; n is 1 or 2;
A is —CR⁴R⁵— or —CHR^{a}—CHR^{b}—;
   R⁴ is selected from the group consisting of (i) hydrogen, (ii) (C₁-C₇)alkyl, (iii) (C₃-C₈)cycloalkyl, (iv) 4 to 10 member heterocycloalkyl, (v) aryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl, (vi) heteroaryl, optionally substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl, and (vii) LR⁶
   wherein:
   L is selected from the group consisting of —CH₂—, —NR⁷—, and —O—; R⁶ is aryl, heteroaryl, (C₁-C₈)alkyl, (C₃-C₈)cycloalkyl, 4 to 10 member heterocycloalkyl, or (C₁-C₈)alkoxy, each of which optionally may be substituted with one to three substituents, the substituents being independently selected from the group consisting of (C₁-C₄)alkyl, (C₁-C₄)alkoxy, halo, (C₁-C₄)haloalkyl, (C₁-C₄)haloalkoxy, (C₃-C₆)cycloalkyl, cyano, carboxy, and carbamoyl; and
   R⁷ is hydrogen, methyl or ethyl;
   R⁵ is selected from the group consisting of hydrogen, hydroxyl, (C₁-C₄)alkoxy, halogen, and (C₁-C₆)alkyl; or R⁴ and R⁵, together with the carbon to which they are attached, form a cycloalkyl or heterocycloalkyl ring that optionally incorporates an oxo group and is optionally substituted with (C₁-C₈)alkyl, (C₃-C₈)cycloalkyl, halo, (C₁-C₈)alkoxy, or (C₁-C₃)haloalkyl;
R^{a} is (C₁-C₄)alkoxy or R⁸—O—C(O)—, wherein R⁸ is (C₁-C₄)alkyl; and R^{b} is aryl, heteroaryl, or heterocycloalkyl, optionally substituted with halo, (C₁-C₈)alkyl, (C₃-C₈)cycloalkyl, (C₁-C₈)alkoxy, or (C₁-C₃)haloalkyl; or R^{a} and R^{b}, together with the carbons to which they are attached, form a cycloalkyl or heterocycloalkyl ring that optionally incorporates an oxo group and is optionally substituted with (C₁-C₈)alkyl, (C₃-C₈)cycloalkyl, halo, (C₁-C₈)alkoxy, or (C₁-C₃)haloalkyl.
   Embodiment 67. The method of any one of embodiments 1-12, wherein the PDE9 inhibitor is represented by a compound of formula (LIII),

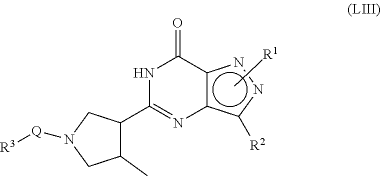

wherein
   R¹ represents hydrogen atom or methyl;
   when R¹ represents hydrogen atom, then R² represents cyclopentyl, tetrahydro-pyranyl, cyclohexyl, or cyclohexyl substituted with 1 or 2 halogen atoms;
   when R¹ represents methyl, then R² represents cyclopentyl;
   R³ is selected from the group consisting of: -phenyl unsubstituted or substituted with 1 to 3 substituents selected from F, Cl, Br, I, and OCH₃; and
   6- to 10-membered heteroaryl with 1 to 3 heteroatoms selected independently from 0, N and S; and Q represents C₁-C₃-alkylene group, which is unsubstituted or substituted with 1 to 3 C₁-C₃-alkyl groups; and their salts, including pharmaceutically acceptable salts, and including optically active diastereoisomers and their mixtures.
      Embodiment 68. The method of any one of embodiments 1-12, wherein the PDE9 inhibitor is represented by a compound of formula (LIV),

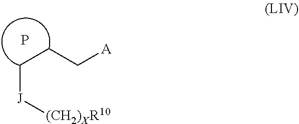

a stereoisomer or prodrug thereof, or a pharmaceutically acceptable salt of said compound, stereoisomer, or prodrug, wherein:
A is:

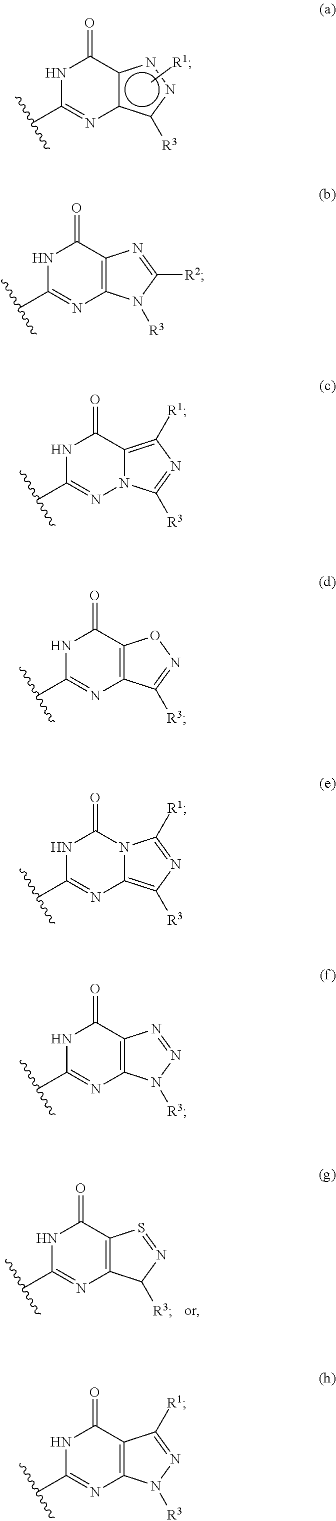

P, including the carbon atoms to which it is attached, is (C₃-C₈)cycloalkyl, (C₃-C₈)heterocycloalkyl, aryl, or heteroaryl; optionally and independently substituted with from 1 to 3 substituents independently selected from halogen, (C₁-C₅)alkyl, (C₁-C₅)alkoxy, and trifluoromethyl;
J is O, S, —N(R¹⁵)—, —N(R¹⁵)CO—, —CON(R¹⁵)—, —SO₂N(R¹⁵)—, or —N(R¹⁵)SO₂—;
x is 0, 1, 2, 3, 5, or 6;
R¹⁰ is —CO₂H, —CONR³⁰R³¹, —NR³⁰R³¹, or —N(R¹⁵)SO₂R⁴⁰;
   R¹ and R² are independently H or (C₁-C₃)alkyl;
   R³ is (C₁-C₈)alkyl, (C₃-C₈)cycloalkyl, (C₃-C₈)cycloalkyl-methyl, (C₃-C₈)heterocycloalkyl, (C₃-C₈)heterocycloalkyl-methyl, aryl, or heteroaryl; optionally and independently substituted with from 1 to 3 substituents independently selected from halogen, hydroxy, oxo, (C₁-C₅)alkyl, and (C₁-C₅)alkoxy;
   R¹⁵ is H or (C₁-C₅)alkyl;
   R³⁰ and R³¹ are taken separately and are independently H, (C₁-C₅)alkyl, (C₃-C₈)cycloalkyl, (C₃-C₈)heterocycloalkyl, aryl, or heteroaryl, wherein said R³⁰ and R³¹ are optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰; or
   R³⁰ and R³¹ are taken together with the nitrogen atom to which they are attached to form a 5- to 8-membered heterocycloalkyl ring, said ring optionally having 1 additional heteroatom independently selected from N, O, and S, wherein said 5- to 8-membered heterocycloalkyl ring is optionally and independently substituted with from 1 to 3 substituents independently selected from halogen, oxo, (C₁-C₅)alkyl, —CO₂R⁴⁰, —COR⁴⁰, —OR⁴⁰, —CONR⁵⁰R⁵¹, —NR⁵⁰R⁵¹, and —SO₂R⁴⁰;
   R⁴⁰ is H, (C₁-C₅)alkyl, (C₃-C₈)cycloalkyl, (C₃-C₈)heterocycloalkyl, aryl, or heteroaryl;
   R⁵⁰ and R⁵¹ are taken separately and are independently H, (C₁-C₅)alkyl, (C₃-C₈)cycloalkyl, (C₃-C₈)heterocycloalkyl, aryl, or heteroaryl; or
   R⁵⁰ and R⁵¹ are taken together with the nitrogen atom to which they are attached to form a 5- to 8-membered heterocycloalkyl ring, said ring optionally having 1 additional heteroatom independently selected from N, O, and S.
      Embodiment 69. The method of any one of embodiments 1-12, wherein the PDE9 inhibitor is represented by a compound of formula (LV),

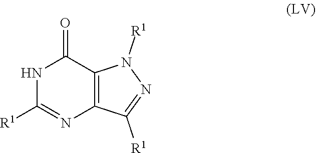

or a pharmaceutically acceptable salt, solvate or prodrug thereof, wherein:
R¹ is H or (C₁-C₆)alkyl;
R² is (C₁-C₆)alkyl, straight chain or branched chain, (C₃-C₇)cycloalkyl or heteroaryl; R³ is (C₁-C₆)alkyl, straight chain or branched chain, optionally substituted by 1-2 groups each independently selected from Ar, (C₃-C₇)cycloalkyl, OAr, SAr, NC(O)(C₁-C₆)alkyl, heteroaryl, xanthene, and naphthalene;
Ar is a group of formula

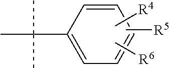

wherein R⁴, R⁵ and R⁶ are each independently selected from H, halo, phenoxy, phenyl, CF₃, OCF₃, S(C₁-C₆)alkyl, (C₁-C₆)alkyl, O(C₁-C₆)alkyl, said alkyl optionally substituted by a heteroaryl group or by a phenyl group, wherein said phenyl group is optionally substituted by 1-3 groups selected from halo, CF₃, OCF₃ and (C₁-C₆)alkyl; or wherein R⁴ and R⁵ may combine to form a (C₂-C₃)alkyl link, wherein said link may optionally incorporate a heteroatom selected from O, S and N; and heteroaryl is aromatic 5-6 membered heterocycle containing 1-3 heteroatoms, each independently selected from O, S and N, said heterocycle optionally substituted by 1-3 substituents, each independently selected from (C₁-C₆)alkyl, halo and phenyl, said phenyl optionally substituted by 1-3 groups selected from halo and (C₁-C₆)alkyl; with the proviso that when R¹ is —CH₃, R² cannot be —CH₂CH₂CH₃.
   Embodiment 70. The method of any one of embodiments 1-12, wherein the PDE9 inhibitor is represented by a compound of formula (LVI) or a pharmaceutically acceptable salt, an isomer, a deuterated compound, a metabolite or a prodrug thereof;

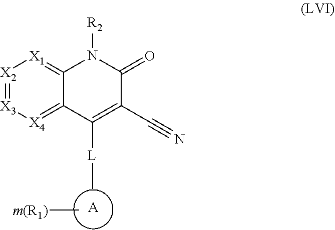

wherein X₁, X₂ and X₄ are each independently selected from CR′ and N; X₃ is selected from CR₃ and N, and at least one of X₁, X₂, X₃ and X₄ is N,
wherein the N heteroatom may be optionally oxidized to

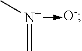

R′ and R₃, at each occurrence, are each independently selected from hydrogen, deuterium, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, 5-6 membered heteroaryl, aryl, C₁₋₆ alkylcarbonyl, aminocarbonyl, C₁₋₆ alkylaminocarbonyl, (C₁₋₆ alkyl)₂aminocarbonyl, 4-6 membered heterocyclylcarbonyl and 5-6 membered heteroaryl-oxy, wherein the C₁₋₆ alkyl, C₁₋₆ alkoxy, C₁₋₆ alkylamino, (C₁₋₆ alkyl)₂amino, halogenated C₁₋₆ alkoxy, C₂₋₈ alkenyl, C₂₋₈ alkynyl, C₁₋₆ alkylsulfonyl, C₁₋₆ alkylthio, C₃₋₆ cycloalkyl, 4-6 membered heterocyclyl, 5-6 membered heteroaryl, aryl, C₁₋₆ alkylcarbonyl, aminocarbonyl, C₁₋₆ alkylaminocarbonyl, (C₁₋₆ alkyl)₂aminocarbonyl, 4-6 membered heterocyclylcarbonyl and 5-6 membered heteroaryl-oxy are unsubstituted or optionally substituted with one or more groups independently selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino, C₁₋₆ alkylsulfonylamino, C₁₋₆ alkylcarbonyloxy, C₃₋₆ cycloalkyl, C₃₋₆ cycloalkylcarbonyloxy, C₂₋₈ alkynyl, halogenated C₁₋₆ alkyl, C₂₋₈ alkenyl, halogenated C₁₋₆ alkoxy,

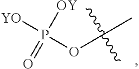

4-6 membered heterocyclyl unsubstituted or optionally substituted with one or more independent substituents, and heteroaryl unsubstituted or optionally substituted with one or more independent substituents;
the substituents for the aforementioned 4-6 membered heterocyclyl optionally substituted with one or more independent substituents and heteroaryl optionally substituted with one or more independent substituents are selected from hydroxy, amino, carboxyl, cyano, nitro, halogen, C₁₋₆ alkyl and C₁₋₆ alkoxy;
Y is selected from metal ions and organic ammonium ions, and is preferably Na⁺, K⁺ or NH₄⁺;
L is a bond or —NH—(CH₂)t-, wherein t is 0, 1, 2 or 3;
ring A is 3-12 membered heterocyclyl, 5-10 membered heteroaryl, 3-12 membered cycloalkyl or 3-12 membered cycloalkenyl, wherein the 3-12 membered heterocyclyl has heteroatoms selected from one of or any combinations of O, S and N, the S atom may be optionally oxidized to S(O) or S(O)₂, the C atom may be optionally oxidized to C(O), the N heteroatom may be optionally oxidized to

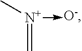

and the 5-10 membered heteroaryl has heteroatoms selected from one of or any combinations of O, S and N;
each R₁ is independently selected from hydrogen, deuterium, hydroxy, amino, carboxyl, cyano, nitro, halogen, C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂₋₈ alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl, wherein the C₁₋₆ alkyl, C₁₋₆ alkoxy, C₁₋₆ alkylamino, (C₁₋₆ alkyl)₂amino, halogenated C₁₋₆ alkyl, halogenated C₁₋₆ alkoxy, C₂-8 alkenyl, C₂₋₈ alkynyl, C₁₋₆ alkylsulfonyl, C₁₋₆ alkylthio, 3-12 membered cycloalkyl, 3-12 membered cycloalkenyl, 3-12 membered heterocyclyl, aryl and 5-10 membered heteroaryl are unsubstituted or optionally substituted with a group selected from hydroxy, amino, carboxyl, cyano, nitro, halogen, C₁₋₆ alkyl, C₁₋₆ alkoxy, C₁₋₆ alkoxy C₁₋₆ alkoxy, C₁₋₆ alkylamino, (C₁₋₆ alkyl)₂amino, C₁₋₆ alkylcarbonylamino and C₁₋₆ alkylsulfonylamino;
m is 0, 1, 2 or 3; and
R² is selected from hydrogen, C₁₋₆ alkyl, C₂₋₈ alkenyl, C₂₋₈ alkynyl and halogenated C₁₋₆ alkyl;
with the proviso that
(1) when X₂ is N, X₁ is CH, X₃ is CR₃, and X₄ is CH, ring A is

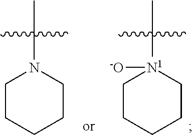

(i) when

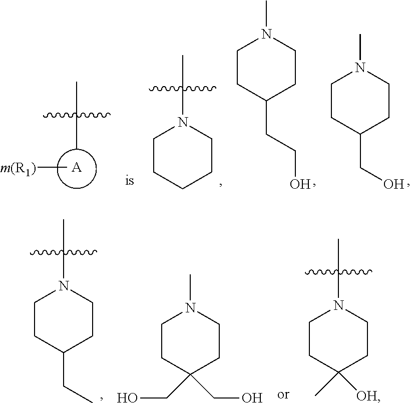

R³ is not H;

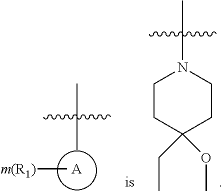

(ii) when

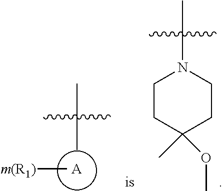

R³ is not Cl;
(iii) when

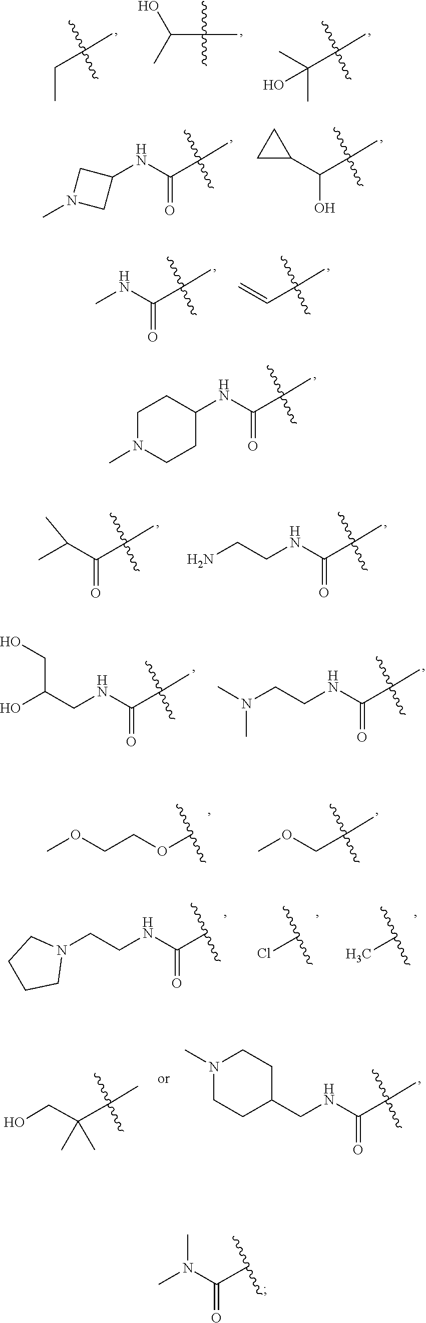

R³ is not H,
(2) when X₂ and X₄ are each N, X is CH X₃ is CR₃ ring A is

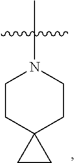 [SMILES: *N1CCC2(CC1)CC2]

and m is 0, R₃ is not methylthio;
(3) when X₄ is N, X₁ and X₂ are each CH, X₃ is CR₃, ring A is

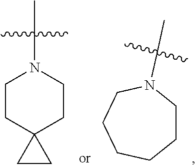

and m is 0, R₃ is not hydrogen; and
(4) when X₁ is N, X₂ and X₄ are CR′, X₃ is CR₃, ring A is pyrrolidinyl or

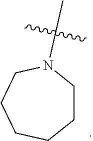 [SMILES: *N1CCCCCC1]

and m is 0, R² is not H or C₁₋₆ alkyl.
Preferably, when X₂ is N, X₁ is CH, X₃ is CR₃, X₄ is CH, and

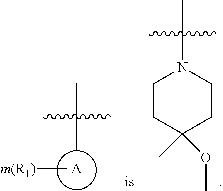

R₃ is selected from isopropyl, cyclopropyl, hydroxymethyl,

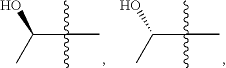

C₁₋₆ alkylcarbonyl, C₁₋₆ alkylcarbonyloxy C₁₋₆ alkyl, C₁₋₆ alkyl substituted with

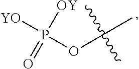

C₃₋₆ cycloalkylcarbonyloxy C₁₋₆ alkyl and deuterated C₁₋₆ alkyl;
preferably, when X₂ is N, X₁ is CH, X₃ is CR₃ and X₄ is CH, ring A is

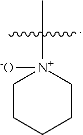

Embodiment 71. The method of any one of embodiments 1-12, wherein the PDE9 inhibitor is represented by a compound of formula (LVII) (i.e. a 7H-imidazo[1,5-a]pyrazin-8-one backbone),

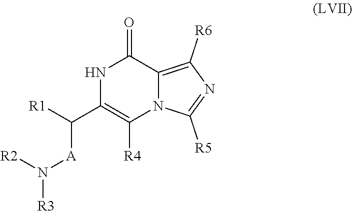

wherein R2 is cyclized with either R1 or R3,
wherein R1, R2 and R3 are
R1, when cyclized with R2, is

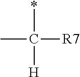

wherein R7 is selected from the group consisting of H, —CH₃, —C₂H₅, and —C₃H₇,
wherein * denotes the cyclization point, and
R1, when not cyclized, is selected from the group consisting of
   H and

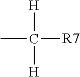

wherein R7 is selected from the group consisting of H, —CH₃, —C₂H₅, and —C₃H₇
R2 is a compound selected from the group consisting of

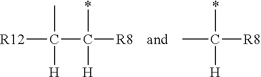

wherein R8 and R12 independently are selected from the group consisting of H, —CH₃, —C₂H₅, and —C₃H₇
   wherein * denotes the cyclization point, and
R3, when cyclized with R2, is

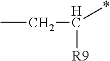

wherein * denotes the cyclization point, and
wherein R9 is selected from the group consisting of H, C₁-C₆ alkyl, branched C₃-C₆ alkyl, C₃-C₆ cycloalkyl, C₆-C₁₀ aryl, substituted C₆-C₁₀ aryl, C₃-C₉heteroaryl, substituted C₃-C₉heteroaryl, C₁-C₆ alkoxy, branched C₃-C₆ alkoxy, C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R3, when not cyclized, is

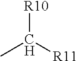

wherein
   R10 is selected from the group consisting of H, —CH₃, and —C₂H₅; and
   R11 is selected from the group consisting of C₆-C₁₀ aryl, substituted C₆-C₁₀ aryl, C₃-C₉heteroaryl, substituted C₃-C₉ heteroaryl
R4 is selected from the group consisting of hydrogen, —CH₃, —C₂H₅, —C₃H₇, —CF₃, —CN, F and Cl;
R5 is selected from the group consisting of C₆-C₁₀ aryl, substituted C₆-C₁₀ aryl, C₃-C₉heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R6 is selected from the group consisting of hydrogen, F, Cl, CN, —CH₃, —C₂H₅, —C₃H₇, and —CF₃;
A is absent or —CH₂—
and tautomers and pharmaceutically acceptable acid addition salts thereof, and polymorphic forms thereof.
   Embodiment 72. The method of any one of embodiments 1-12, wherein the PDE9 inhibitor is represented by a compound of formula (LVIII) or a pharmaceutically acceptable salt, solvate, or polymorphic thereof:

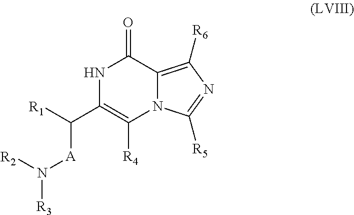

wherein R² is cyclized with either R¹ or R³; wherein R¹, R², and R³ are R¹, when cyclized with R² is

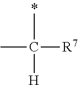

wherein R⁷ is selected from the group consisting of H, —CEE, —C2H5, and C3H7;
wherein * denotes the cyclization point; and
R¹, when not cyclized, is selected from the group consisting of H and

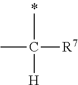

wherein R⁷ is selected from the group consisting of H, —CH₃, —C₂H₅, and C₃H₇;
R² is a compound selected from the group consisting of

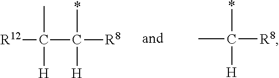

wherein R⁸ and R¹² independently are selected from the group consisting of H, —CH₃, —C₂H₆, and —C₃H₇,
wherein * denotes the cyclization point; and
R³, when cyclized with R² is

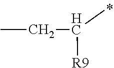

wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryl oxy; and
R³, when not cyclized, is

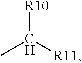

wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C₃-C₉ heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R⁶ is selected from the group consisting of hydrogen, F, C₁, CN, —CH₃, —C₂H₅, —C₃H₇, and —CF₃; and
A is absent or —CH₂.
   Embodiment 73. The method of embodiment 72, wherein the PDE9 inhibitor is

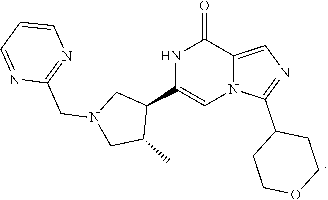 [SMILES: C[C@@H]1CN(Cc2ncccn2)C[C@H]1c1cn2c(C3CCOCC3)ncc2c(=O)[nH]1]

Embodiment 74. The method of any one of embodiments 1-12, wherein the PDE9 inhibitor is represented by a compound of formula (LIX),

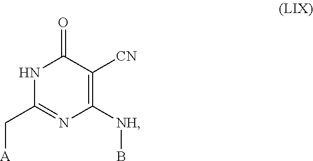

in which
   A is C₁-C₈-alkyl, C₃-C₈-cycloalkyl, tetrahydrofuryl or tetrahydropyranyl, which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
      where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by one or more radicals selected from the group of hydroxy, cyano, halogen, hydroxycarbonyl and a group of the formula —NR³R⁴,
         where
         R³ and R⁴ are independently of one another hydrogen or C₁-C₆-alkyl,
         or
         R³ and R⁴ together with the nitrogen atom to which they are bonded are 5- to 8-membered heterocyclyl,
   B is phenyl or heteroaryl which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, nitro, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
      where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by a radical selected from the group of hydroxy, cyano, halogen, hydroxycarbonyl and a group of the formula —NR³R⁴,
         where
         R³ and R⁴ have the abovementioned meanings,
and the salts, solvates and/or solvates of the salts thereof.
   Embodiment 75. The method of any one of embodiments 1-12, wherein the PDE9 inhibitor is represented by a compound of formula (LX),

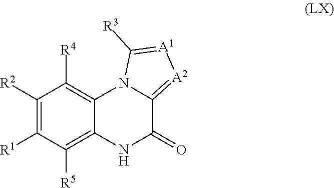

wherein
A¹ is N and A² is CR⁶,
one of R¹ and R² is hydrogen; halogen; or lower alkyl, —O-lower alkyl, or cycloalkyl, each of which may be substituted with one or more substituents selected from the group consisting of —OH, —O-lower alkyl, —NH₂, —NH-lower alkyl, —N(lower alkyl)₂, and a monocyclic nitrogen-containing hetero ring which may be substituted with lower alkyl; and the other is a group of formula (LXI),

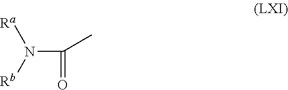

R³ is lower alkyl, cycloalkyl, cycloalkenyl, aryl, heteroaryl, or a saturated hetero ring, each of which may be substituted with one or more substituents selected from the group consisting of halogen, lower alkyl, cycloalkyl, —OH, oxo, —O-lower alkyl, —COOH, —CO—O-lower alkyl, —CO—O-lower alkenyl, —CO—O-lower alkynyl, —CO—O-lower alkylene-O-lower alkyl, —CO—O-lower alkylene-aryl, —CO—O-lower alkylene-O-aryl, —CO—NH₂, —CO—NH-lower alkyl, —CO—N(lower alkyl)₂, —CO—N(lower alkyl)-aryl, —CO—N(lower alkyl)-(lower alkylene-aryl), —CO—NH-lower alkylene-OH, —CO—NH-lower alkylene-CO₂H, and a monocyclic sulfur-containing saturated hetero ring,
R⁴ and R⁵ are the same or different, and each is hydrogen or lower alkyl,
R⁶ is hydrogen or lower alkyl, and
R^{a} and R^{b} are combined with the adjacent nitrogen atom to form a polycyclic nitrogen-containing hetero ring, which may be substituted with one or more substituents selected from the group consisting of halogen; —OH; oxo; —O-lower alkyl; cyano; nitro; halogeno-lower alkyl; cycloalkyl; aryl which may be further substituted with a group selected from a group G₁; a hetero ring which may be further substituted with a group selected from a group G₂; lower alkylene-(aryl which may be further substituted with a group selected from a group G₁); lower alkylene-SO₂—NR⁷R⁸; lower alkylene-hetero ring; lower alkyl which may be further substituted with —OH, —O-lower alkyl, cyano, or cycloalkyl; —COOH; —CO—O-lower alkyl; —CO—O-lower alkenyl; —CO—O-lower alkynyl; —CO—O-lower alkylene-O-lower alkyl; —CO—O-lower alkylene-aryl; —CO—O-lower alkylene-O-aryl; —CO—NH₂; —CO—NH-lower alkyl; —CO—N lower alkyl)₂, —CO—N(lower alkyl)-aryl; —CO—N(lower alkyl)-(lower alkylene-aryl); —CO—NH-lower alkylene-OH; —CO—NH-lower alkylene-CO₂H; —NH—SO₂—R⁹; —SO₂—NR⁷R⁸; and a monocyclic nitrogen-containing hetero ring, wherein R⁷ and R⁸ are the same or different, and each is hydrogen or lower alkyl, R⁹ is lower alkyl, or aryl which may be substituted with lower alkyl; group G₁ contains halogen, lower alkyl, halogeno-lower alkyl, —OH, —O-lower alkyl, —O-lower alkylene-aryl, —O-lower alkylene-hetero ring, —O-halogeno-lower alkyl, aryl, and a hetero ring, and group G₂ contains halogen, lower alkyl, halogeno-lower alkyl, —OH, —O-lower alkyl, —O-lower alkylene-aryl, —O-lower alkylene-hetero ring, —O-halogeno-lower alkyl, aryl, and a hetero ring,
or a pharmaceutically acceptable salt thereof.
   Embodiment 76. The method of any one of embodiments 1-12, wherein the PDE9 inhibitor is represented by a compound of formula (LXII),

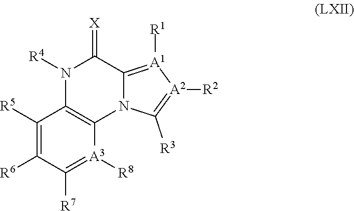

in the formula,
   R¹ and R² each independently stands for hydrogen; halogen; C₁₋₆ alkyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group; acyl which is optionally substituted with hydroxy, C₁₋₆ alkyl, C₂₋₆ alkenyl, C₂₋₆ alkynyl, C₁₋₆ haloalkyl having 1-9 halogen atoms, C₁₋₆ alkoxy, C₁₋₆haloalkoxy having 1-9 halogen atoms, amino, carbocyclic group or heterocyclic group; amino which is optionally substituted with 1-2 substituents selected from C₁₋₆ alkyl, C₂₋₆ alkenyl, C₂-6 alkynyl, alkanoyl, carbocyclic group and heterocyclic group; hydroxy; or pyrimidinyl which is optionally substituted with halogen, C₁₋₆ alkyl, C₁₋₆ alkoxy, C₁₋₆ haloalkoxy having 1-9 halogen atoms, nitro or amino,
   R³ stands for C₁₋₉ alkyl, C₂₋₉ alkenyl or C₂₋₉ alkynyl which are optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino group may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); aryl, saturated carbocyclic group or saturated heterocyclic group each of which is optionally substituted with halogen, hydroxy, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with halogen, hydroxy, C₁₋₆ alkoxy, C₁₋₆ haloalkoxy having 1-9 halogen atoms, carboxy, C₁₋₆ alkoxycarbonyl, alkanoyl, amino, amido, carbamoyl, carbocyclic group or heterocyclic group, independently of each other), C₁₋₆ haloalkoxy having 1-9 halogen atoms, carboxy, C₁₋₆ alkoxycarbonyl, alkanoyl, amino, amido, carbamoyl, carbocyclic group or heterocyclic group; carboxy; C₁₋₆ alkoxycarbonyl (here the C₁₋₆ alkoxy moiety in the C₁₋₆ alkoxycarbonyl may further be substituted with hydroxy, halogen, C₁₋₆ alkoxy, C₁₋₆ haloalkoxy having 1-9 halogen atoms, carboxy, C₁₋₆ alkoxycarbonyl, amino, amido, carbamoyl, carbocyclic group or heterocyclic group); amido (here the amino moiety in the amido may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group); or carbamoyl (here the amino moiety in the carbamoyl may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group),
   R⁴ stands for hydrogen; hydroxy; C₁₋₆ alkyl which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; or amino which is optionally substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group,
   R⁵ and R⁸ each independently stands for hydrogen; halogen; C₁₋₆ alkyl, C₂₋₆ alkenyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl or oxo; cyano; or nitro,
   R⁶ and R⁷ each independently stands for hydrogen; halogen; C₁₋₆ alkyl, C₂₋₆ alkenyl, C₂₋₆ alkynyl or C₁₋₆ alkoxy each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group or heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); cyano; amino which is optionally substituted with 1-2 substituents selected from C₁₋₆ alkyl, C₂₋₆ alkenyl, C₂₋₆ alkynyl (here the C₁₋₆ alkyl, C₂₋₆ alkenyl and C₂₋₆ alkynyl may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group and heterocyclic group), alkanoyl, carbocyclic group and heterocyclic group (here the carbocyclic group and heterocyclic group each may further be substituted with hydroxy, halogen, C₁₋₆ alkyl, C₂₋₆ alkenyl, C₂₋₆ alkynyl, C₁₋₆ alkoxy, carboxy, C₁₋₆ alkoxycarbonyl, amino, amido or carbamoyl); carbocyclic group or heterocyclic group each of which is optionally substituted with hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, C₁₋₆ alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, amino, amido or carbamoyl; COR⁹; or SO₂R⁹,
   R⁹ stands for hydrogen; hydroxy; C₁₋₆ alkyl, C₂₋₆ alkenyl, C₂₋₆ alkynyl or C₁₋₆ alkoxy, each of which is optionally substituted with hydroxy, halogen, C₁₋₆ alkoxy, C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, oxo, carbocyclic group or heterocyclic group (here the carbocyclic group and heterocyclic group each may further be substituted with hydroxy, halogen, C₁₋₆ alkyl, C₂-6 alkenyl, C₂₋₆ alkynyl, C₁₋₆ alkoxy, carboxy, C₁₋₆ alkoxycarbonyl, amino, amido or carbamoyl); amino which may be substituted with 1-2 substituents selected from C₁₋₆ alkyl, C₂₋₆ alkenyl, C₂-6 alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl, oxo, carbocyclic group or heterocyclic group), C₁₋₆ haloalkyl having 1-9 halogen atoms, alkanoyl, carbocyclic group and heterocyclic group (here the carbocyclic group and heterocyclic group each may further be substituted with hydroxy, halogen, C₁₋₆ alkyl, C₂₋₆ alkenyl, C₂-6 alkynyl, C₁₋₆ alkoxy, carboxy, C₁₋₆ alkoxycarbonyl, amino, amido or carbamoyl); or aziridin-1-yl, azetidin-1-yl, pyrrolidin-1-yl, piperidin-1-yl, piperazin-1-yl, morpholin-1-yl or pyrazol-1-yl, each of which may be substituted with 1-2 substituents selected from hydroxy, halogen, C₁₋₆ alkyl, C₂₋₆ alkenyl, C₂₋₆ alkynyl, C₁₋₆ alkoxy (here the C₁₋₆ alkyl, C₂₋₆ alkenyl, C₂₋₆ alkynyl and C₁₋₆ alkoxy may further be substituted with, independently of each other, hydroxy, halogen, C₁₋₆ alkoxy, C₁₋₆ haloalkoxy having 1-9 halogen atoms, carboxy, C₁₋₆ alkoxycarbonyl, alkanoyl, amino, amido, carbamoyl, oxo, carbocyclic group or heterocyclic group), C₁₋₆ haloalkoxy having 1-9 halogen atoms, carboxy, C₁₋₆ alkoxycarbonyl, alkanoyl, amino (here the amino may further be substituted with 1-2 substituents selected from C₁₋₆ alkyl, C₂₋₆ alkenyl, C₂₋₆ alkynyl, C₁₋₆ haloalkyl having 1-9 halogen atoms, alkanoyl, carbocyclic group and heterocyclic group), amido, carbamoyl, oxo, carbocyclic group and heterocyclic group (here the carbocyclic group and heterocyclic group each may further be substituted with hydroxy, halogen, C₁₋₆ alkyl, C₂-6 alkenyl, C₂₋₆ alkynyl, C₁₋₆ alkoxy, carboxy, C₁₋₆ alkoxycarbonyl, amino, amido or carbamoyl), X stands for S or O,
A¹, A² and A³ stand for N or C, independently of each other, with the proviso that R¹, R² and R⁸ are respectively absent where A¹, A² and A³ respectively stand for N, or salts thereof are provided.
   Embodiment 77. The method of any one of embodiments 1-12, wherein the PDE9 inhibitor is represented by a compound of formula (LXIII),

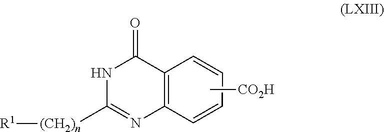

in the formula,
   R¹ stands for phenyl or aromatic heterocyclic group which are optionally substituted with 1-3 substituents selected from halogen, C₁₋₆ alkyl, C₁₋₆ haloalkyl containing 1-6 halogen atoms and C₁₋₆ alkoxy; and
   n is an integer of 1-3
   or salts thereof.
      Embodiment 78. The method of any one of embodiments 1-12, wherein the PDE9 inhibitor is represented by a compound of formula (LXIV),

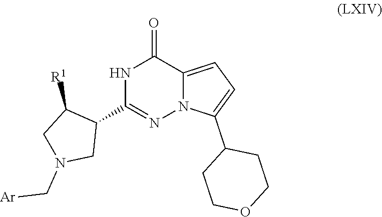

where Rⁱ represents H,

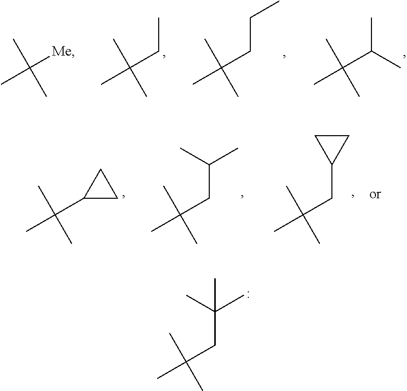

Ar stands for

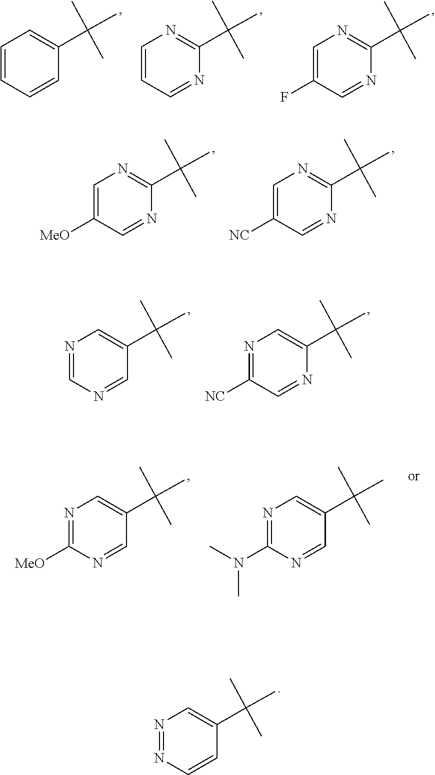

Embodiment 79. The method of any one of embodiments 1-12, wherein PDE9 inhibitor is represented by a compound of formula (LXV),

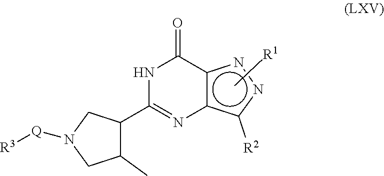

wherein
R¹ represents hydrogen atom or methyl;
when R¹ represents hydrogen atom, then R² represents cyclopentyl, tetrahydro-pyranyl, cyclohexyl, or cyclohexyl substituted with 1 or 2 halogen atoms;
when R¹ represents methyl, then R² represents cyclopentyl;
R³ is selected from the group consisting of -phenyl unsubstituted or substituted with 1 to 3 substituents selected from F, Cl, Br, I, and OCH₃; and -6- to 10-membered heteroaryl with 1 to 3 heteroatoms selected independently from O, N and S; and Q represents C1-C3-alkylene group, which is unsubstituted or substituted with 1 to 3 C1-C3-alkyl groups; and their salts, including pharmaceutically acceptable salts, and including optically active diastereoisomers and their mixtures.
   Embodiment 80. The method of any one of embodiments 1-12, wherein PDE9 inhibitor is represented by a compound of formula (LXVI),

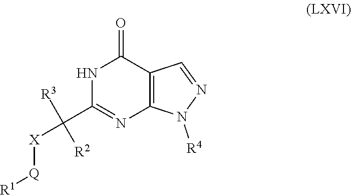

wherein:
   R¹ is selected from the group consisting of
   phenyl unsubstituted or substituted with 1 to 3 substituents selected from F, C₁, Br, I, CN, —O—C₁-C₃-alkyl, fluorinated —O—C₁-C₃-alkyl, —(CH₂)ₘOH, and 5-membered heterocyclic group with 1 or 2 heteroatoms selected from N, O and S; and -6- or 10-membered heteroaryl with 1 to 3 heteroatoms selected from O, N and S;
   R² and R³ are independently of each other represent H atom or straight or branched C₁-C₃ alkyl;
   R⁴ is selected from the group consisting of 4- to 6-membered cycloalkyl, wherein one of carbon atoms can be replaced by 0 atom, and which is unsubstituted or substituted with one or two halogen atoms; and straight or branched C₁-C₄ alkyl;
   Q represents a bond or C₁-C₃-alkylene, which can be optionally substituted by one to three C₁-C₃-alkyls;
   X is selected from the group consisting of O, NR⁵, and S(O)ₚ;
   R⁵ represents H atom or C₁-C₃ alkyl;
   m is 1, 2 or 3; p is 0, 1 or 2;
   and salts thereof.
      Embodiment 81. The method of any one of embodiments 1-12, wherein PDE9 inhibitor is represented by a compound of formula (LXVII)

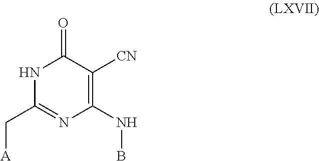

in which
A is C₁-C₈-alkyl, C₃-C₈-cycloalkyl, tetrahydrofuryl or tetrahydropyranyl, which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio,
where C₁-C₆-alkyl, C₁-C₆-alkoxy, C₁-C₆-alkylamino, C₁-C₆-alkylaminocarbonyl, C₁-C₆-alkoxycarbonyl, C₁-C₆-alkylcarbonyl, C₁-C₆-alkylsulfonyl and C₁-C₆-alkylthio are optionally substituted by one or more radicals selected from the group of hydroxy, cyano, halogen, hydroxycarbonyl and a group of the formula —NR³R⁴,
   where
   R³ and R⁴ are independently of one another hydrogen or C₁-C₆-alkyl, or
   R³ and R⁴ together with the nitrogen atom to which they are bonded are 5- to 8-membered heterocyclyl,
B is phenyl or heteroaryl which are optionally substituted by up to 3 radicals independently of one another selected from the group of C₁-C₆-alkyl, C₁-C₆-alkoxy, hydroxycarbonyl, cyano, trifluoromethyl, trifluoromethoxy, amino, nitro, hydroxy, C₁-C₆-alkylamino, halogen, C₁-C₆-alkylaminocarbonyl, C₁-C₆-alkoxycarbonyl, C₁-C₆-alkylcarbonyl, C₁-C₆-alkylsulfonyl and C₁-C₆-alkylthio, where C₁-C₆-alkyl, C₁-C₆-alkoxy, C₁-C₆-alkylamino, C₁-C₆-alkylaminocar-bonyl, C₁-C₆-alkoxycarbonyl, C₁-C₆-alkylcarbonyl, C₁-C₆-alkylsul-fonyl and C₁-C₆-alkylthio are optionally substituted by a radical selected from the group of hydroxy, cyano, halogen, hydroxycarbonyl and a group of the formula —NR³R⁴,
   where
   R³ and R⁴ have the abovementioned meanings,
or salts thereof.
   Embodiment 82. The method of any one of embodiments 1-12, wherein PDE9 inhibitor is a compound having a structure of:

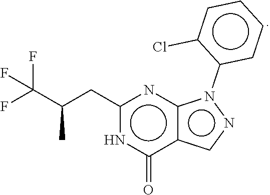

Embodiment 83. The method of any one of embodiments 1-12, wherein PDE9 inhibitor is a compound having a structure of:

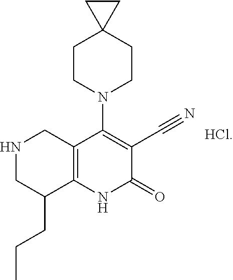 [SMILES: CCCC1CNCc2c1[nH]c(=O)c(C#N)c2N1CCC2(CC1)CC2]

Embodiment 84. The method of any one of embodiments 1-12, wherein PDE9 inhibitor is represented by a compound of formula (LXVIII),
in which

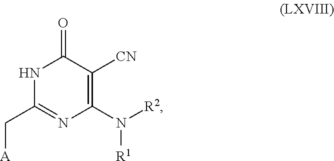

A is phenyl; heteroaryl which is an aromatic monocyclic radical having 5 to 6 ring atoms and 1 to 3 heteroatoms selected from S, O and N; or a group of the formula

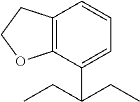

where, when A is phenyl it is substituted by 1 or 2 radicals and when A is heteroaryl it is optionally substituted by up to 2 radicals, where the radicals are independently of one another selected from the group of heteroaryl which is being an aromatic, monocyclic radical having 5 to 6 ring atoms and 1 to 3 heteroatoms selected from S, O and N; halogen; C₁-C₆-alkyl; C₁-C₆-alkoxy; trifluoromethyl; trifluoromethoxy; benzyloxy and benzyl;
   where C₁-C₆-alkyl is optionally substituted by a group of the formula —NR³R⁴ in which R³ is C₁-C₆-alkyl and R⁴ is hydrogen or C₁-C₆-alkoxy(C₁-C₆)alkyl, and heteroaryl is optionally substituted by C₁-C₆-alkoxy,
R¹ is C₃-C₈-cycloalkyl, C₁-C₆-alkyl, C₁-C₆-alkoxy(C₁-C₆)alkyl, benzyl or a group of the formula

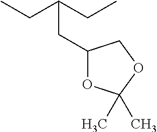

where C₃-C₈-cycloalkyl is optionally substituted by hydroxy, C₁-C₆-alkyl or trifluoromethyl,
C₁-C₆-alkyl is optionally substituted by heteroaryl, C₃-C₈-cycloalkyl or hydroxy, and benzyl is optionally substituted by C₁-C₆-alkoxy or halogen,
R² is hydrogen, or
R¹ and R² together with the nitrogen atom to which they are bonded form a 5- to 6-membered heterocyclyl group which is a monocyclic, saturated or partially unsaturated heterocyclic radical having 5 to 6 ring atoms and 1 to 2 heteroatoms selected from N, O, and S which is optionally substituted by up to 2 substituents independently of one another selected from C₁-C₆-alkyl; hydroxy; cyano; oxo; heteroaryl which is an aromatic, monocyclic radical having 5 to 6 ring atoms and 1 to 3 heteroatoms selected from S, O and N; benzyl; formyl; C₁-C₆-alkylcarbonyl; and the following groups

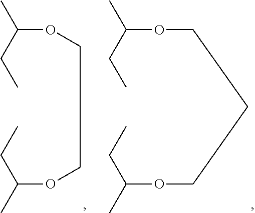

which are linked via the two oxygen atoms to one of the carbon atoms in the heterocycle, where C₁-C₆-alkyl is optionally substituted by hydroxy or heteroaryl which is an aromatic, monocyclic radical having 5 to 6 ring atoms and 1 to 3 heteroatoms selected from S, O and N; or a salt thereof.
   Embodiment 85. The method of any one of embodiments 1-12, wherein PDE9 inhibitor is represented by a compound of formula (AA-I),

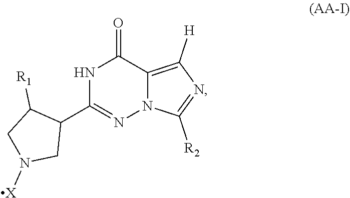

or a pharmaceutically acceptable salt thereof, wherein:
X is selected from the group consisting of a bond, C(O) and S(O)₂;
R¹ is selected from the group consisting of:
   (i) (C₁-C₆) alkyl, which is optionally substituted with one or more R^{a};
   (ii) (C₁-C₆)haloalkyl, which is optionally substituted with one or more R^{a};
   (iii) (C₁-C₆) alkoxy, which is optionally substituted with one or more R^{a};
   (iv) (C₃-C₇) cycloalkyl, which is optionally substituted with one or more R^{b};
   (v) (C₃-C₇) cycloalkyl(C₁-C₄) alkyl, wherein the alkyl portion is optionally substituted with one or more R^{a}, and the cycloalkyl portion is optionally substituted with one or more R^{b};
   (vi) (C₃-C₇) cycloalkyloxy, which is optionally substituted with one or more R;
   (vii) heterocycloalkyl, which is optionally substituted with one or more R^{b};
   (viii) heterocycloalkyl(C₁-C₄)alkyl, wherein the alkyl portion is optionally substituted with one or more R^{a}, and the cycloalkyl portion is optionally substituted with one or more R^{b}; and
   (ix) heterocycloalkyloxy, which is optionally substituted with one or more R^{b};
R² is selected from the group consisting of:
   (i) heterocycloalkyl, which is optionally substituted with one or more R^{c};
   (ii) heterocycloalkyl(C₁-C₄)alkyl, wherein the alkyl portion is optionally substituted with one or more R^{a}, and the cycloalkyl portion is optionally substituted with one or more R^{c};
   (iii) (C₃-C₇) cycloalkyl, which is optionally substituted with one or more R^{c};
   (iv) (C₃-C₇) cycloalkyl(C₁-C₄) alkyl, wherein the alkyl portion is optionally substituted with one or more R^{d}, and the cycloalkyl portion is optionally substituted with one or more R^{c};
   (v) phenyl, which is optionally substituted with one or more R^{c};
   (vi) phenyl(C₁-C₄)alkyl, wherein the alkyl portion is optionally substituted with one or more R^{d}, and the phenyl portion is optionally substituted with one or more R^{c};
   (vii) heteroaryl, which is optionally substituted with one or more R^{c};
   (viii) heteroaryl(C₁-C₄)alkyl, wherein the alkyl portion is optionally substituted with one or more R^{d}, and the heteroaryl portion is optionally substituted with one or more R^{c};
   (ix) (C₁-C₆) alkyl, which is optionally substituted with one or more R^{d}; and
   (x) (C₁-C₆)haloalkyl, which is optionally substituted with one or more R^{d};
R³ is selected from the group consisting of:
   (i) heteroaryl, which is optionally substituted with one or more R;
   (ii) heteroaryl(C₁-C₄)alkyl, wherein the alkyl portion is optionally substituted with one or more R^{g}, and the heteroaryl portion is optionally substituted with one or more R;
   (iii) phenyl, which is optionally substituted with one or more R;
   (iv) phenyl(C₁-C₄)alkyl, wherein the alkyl portion is optionally substituted with one or more R^{g}, and the phenyl portion is optionally substituted with one or more R^{f};
   (v) (C₃-C₇) cycloalkyl, which is optionally substituted with one or more R^{h};
   (vi) (C₃-C₇) cycloalkyl(C₁-C₄) alkyl, wherein the alkyl portion is optionally substituted with one or more R^{g}, and the cycloalkyl portion is optionally substituted with one or more R^{h};
   (vii) heterocycloalkyl, which is optionally substituted with one or more R^{h};
   (viii) heterocycloalkyl(C₁-C₄)alkyl, wherein the alkyl portion is optionally substituted with one or more R^{g}, and the cycloalkyl portion is optionally substituted with one or more R^{h};
   (ix) (C₁-C₆) alkyl, which is optionally substituted with one or more R^{g}; and
   (x) (C₁-C₆)haloalkyl, which is optionally substituted with one or more R^{g}; each occurrence of R^{a}, R^{d}, and R^{g} is independently selected from the group consisting of —S(O)₂(C₁-C₄)alkyl, —OH, —C(O)—(C₁-C₄)alkyl, oxo, —CN, (C₁-C₄)alkoxy, (C₁-C₄)haloalkoxy, (C₃-Cy)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]₂, —NH₂, —NH(C₁-C₄)alkyl, —N[(C₁-C₄)alkyl]₂, —S(O)₂NH(C₁-C₄)alkyl, and —S(O)₂N[(C₁-C₄)alkyl]₂;
each occurrence of R^{a}, R^{d} and R^{g} is independently selected from the group consisting of halo, —S(O)₂(C₁-C₄)alkyl, —OH, —C(O)—(C₁-C₄)alkyl, oxo, —CN, (C₁-C₄)alkoxy, (C₁-C₄)haloalkoxy, (C₃-C₇)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]₂, —S(O)₂NH(C₁-C₄)alkyl, and —S(O)₂N[(C₁-C₄)alkyl]₂; each occurrence of R^{b}, R^{c}, and R^{h} is independently selected from the group consisting of halo, —S(O)₂(C₁-C₄)alkyl, —OH, —C(O)—(C₁-C₄)alkyl, —C(O)— O —(C₁-C₆)alkyl, —CN, (C₁-C₆)alkyl, (C₁-C₄)haloalkyl, (C₁-C₄)alkoxy, (C₁-C₄)haloalkoxy, (C₃—Cy)cycloalkyl, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl]₂, —S(O)₂NH(C₁-C₄)alkyl, and —S(O)₂N[(C₁-C₄)alkyl]₂; and
   each occurrence of R^{c} and R^{f} is independently selected from the group consisting of halo; —S(O)₂(C₁-C₄)alkyl; —OH; —C(O)—(C₁-C₄)alkyl; —C(O)— O —(C₁-C₆)alkyl; —CN; —C(O)OH; (C₁-C₆)alkyl that is optionally substituted with —OH or —OCH₃; (C₁-C₄)haloalkyl; (C₁-C₄)alkoxy; (C₁-C₄)haloalkoxy; (C₃-Cy)cycloalkyl, that is optionally substituted with from 1-3 substituents independently selected from the group consisting of (C₁-C₄)alkyl; (C₃-C₆)cycloalkyloxy; (C₁-C₄)alkylthio; (C₃-C₆)cycloalkylthio-; —C(O)NH₂; —C(O)NH(C₁-C₄)alkyl; —C(O)N[(C₁-C₄)alkyl]₂; —NH₂; —NH(C₁-C₄)alkyl; —N[(C₁-C₄)alkyl]₂; —NH—C(O)—(C₁-C₄)alkyl; —S(O)₂NH(C₁-C₄)alkyl; —S(O)₂N[(C₁-C₄)alkyl]2; heterocycloalkyl that is optionally substituted with from 1-3 substituents independently selected from the group consisting of (C₁-C₄)alkyl; phenyl that is optionally substituted with from 1-3 substituents independently selected from the group consisting of —F, —CI, (C₁-C₄)alkyl, —CF₃, —OCH₃, and —CN; and heteroaryl including from 5 to 6 ring atoms independently selected from the group consisting of N, O, and S, wherein said heteroaryl is optionally substituted with from 1-3 substituents independently selected from the group consisting of (C₁-C₄)alkyl, —CF₃, and —OCH₃.
      Embodiment 86. The method of any embodiments 1-12, wherein PDE9 inhibitor is represented by a compound of formula (AA-II),

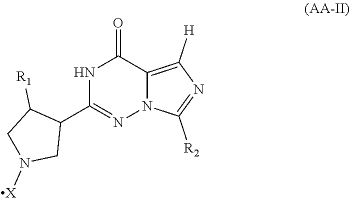

or a pharmaceutically acceptable salt thereof,
wherein:
   X is selected from a bond, C(O) or S(O)₂;
   R₁ is independently selected from the group consisting of H, (C₁-C₆) alkyl, (C₁-C₆) alkoxy, (C₃-C₇) cycloalkyl, (C₃-C₇) cycloalkyl(C₁-C₄) alkyl, (C₃-C₇) cycloalkyloxy, heterocycloalkyl, heterocycloalkyl(C₁-C₄)alkyl and heterocycloalkyloxy, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl];
   R² is independently selected from the group consisting of (C₁-C₆) alkyl, (C₃-C₁₀)cycloalkyl, (C₃-C₇)cycloalkyl(C₁-C₄) alkyl, heterocycloalkyl, heterocycloalkyl(C₁-C₄)alkyl, heteroaryl, heteroaryl(C₁-C₄)alkyl, restricted phenyl and restricted phenyl(C₁—C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl]; and R³ is independently selected from the group consisting of (C₁-C₆) alkyl, (C₃-C₇) cycloalkyl, (C₃-C₇)cycloalkyl(C₁-C₄)alkyl, heterocycloalkyl, heterocycloalkyl(C₁-C₄)alkyl, heteroaryl, heteroaryl(C₁-C₄)alkyl, restricted phenyl and restricted phenyl(C₁-C₄)alkyl, each of which may be optionally substituted with one or more substituents selected from the group consisting of halogen, —S(O)₂(C₁-C₄)alkyl, OH, —C(O)—(C₁-C₄)alkyl, oxo, CN, (C₁-C₆)alkyl, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄alkyl)-C(O)—, (C₁-C₄)alkylsulfonyl-, —S(O)₂NH(C₁-C₄)alkyl, and —S(O)₂N[(C₁-C₄)alkyl(C₁-C₄)alkyl].
      Embodiment 87. The method of any one of embodiments 1-12, wherein PDE9 inhibitor is represented by a compound of formula (AA-III),

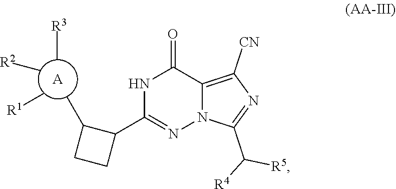

wherein:
A is a pyrazinyl, pyrazolyl, pyridyl, pyridyl-N-oxide, 5-pyrimidinyl, pyridazinyl, pyrrolopyrazolyl, dihydropyrrolopyrazolyl, morpholinyl, oxomorpholinyl, or oxadiazolyl ring;
R¹, R² and R³ as are present are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃,
   (3) —CF₃,
   (4) —CH₂OH,
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} as are present are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl, and
   (7) —CN; or a pharmaceutically acceptable salt thereof.
      Embodiment 88. The method of any one of embodiments 1-12, wherein PDE9 inhibitor is represented by a compound of formula (AA-IV),

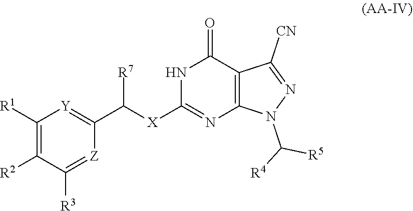

wherein:
X is —N(R⁶)— or —C(R⁸R⁹).
Y is —N═ or —CH═;
Z is —N═ or —CH═;
R¹, R² and R³ are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃;
   (3) —CF₃S
   (4) —CH₂OH,
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl, pyridazinyl, thiazolyl, cyclohexyl or tetrahydropyranyl ring is substituted with R^{1a}, R^{1b} and R^{1c}, wherein R^{1a}, R^{1b} and R^{1c} are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) azetidine, morpholine, piperidine, or pyrrolidine, and
   (8) —CN;
R⁶ is selected from:
   (1) hydrogen, and
   (2) C₁₋₆alkyl;
R⁷ is selected from:
   (1) hydrogen,
   (2) C₁₋₆alkyl, which is unsubstituted or substituted with substituents selected from: hydroxy and fluoro,
   (3) C₂₋₆alkenyl,
   (4) C₃₋₆cycloalkyl, which is unsubstituted or substituted with substituents selected from: hydroxy and fluoro, and
   (5) azetidine, morpholine, piperidine, or pyrrolidine;
or R⁶ and R⁷ and the —CH—N— to which they are attached are joined to form an azetidine or a pyrrolidine ring;
R⁸ and R⁹ are independently selected from:
   (1) hydrogen, and
   (2) C₁₋₆alkyl; or a pharmaceutically acceptable salt thereof.
      Embodiment 89. The method of any one of embodiments 1-12, wherein PDE9 inhibitor is represented by a compound of formula (AA-V),

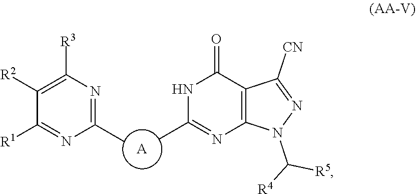

wherein
A is a cyclobutyl ring, which is unsubstituted or or substituted with substituents selected from: fluoro and methyl;
R¹, R² and R³ are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl,
   (7) C₂₋₆alkynyl, and
   (8) —CN;
R⁴ is selected from:
   (1) hydrogen,
   (2) —CH₃;
   (3) —CF₃,
   (4) —CH₂OH.
   (5) —CO₂H, and
   (6) —CH₂CH₃;
R⁵ is a phenyl, pyridyl, pyrazinyl, pyrazolyl, pyrimidinyl, pyridazinyl, thiazolyl, cyclohexyl or tetrahydropyranyl ring, wherein the phenyl, pyridyl, pyrazinyl, pyrazolyl, pyrimidinyl. pyridazinyl, thiazolyl, cyclohexyl or tetrahydropyranyl ring is substituted with R^{1a}, R^{1b} and R^{1c}, wherein R^{1a} R^{1b} and R^{1c} are independently selected from:
   (1) hydrogen,
   (2) halogen,
   (3) hydroxyl,
   (4) C₁₋₆alkyl, which is unsubstituted or substituted with substituents selected from: hydroxy and fluoro,
   (5) —O—C₁₋₆alkyl, which is unsubstituted or substituted with substituents selected from fluoro,
   (6) C₃₋₆cycloalkyl, and
   (7) —CN; pharmaceutically acceptable salt thereof.
      Embodiment 90. The method of any one of embodiments 1-12, wherein PDE9 inhibitor is selected from the group consisting of

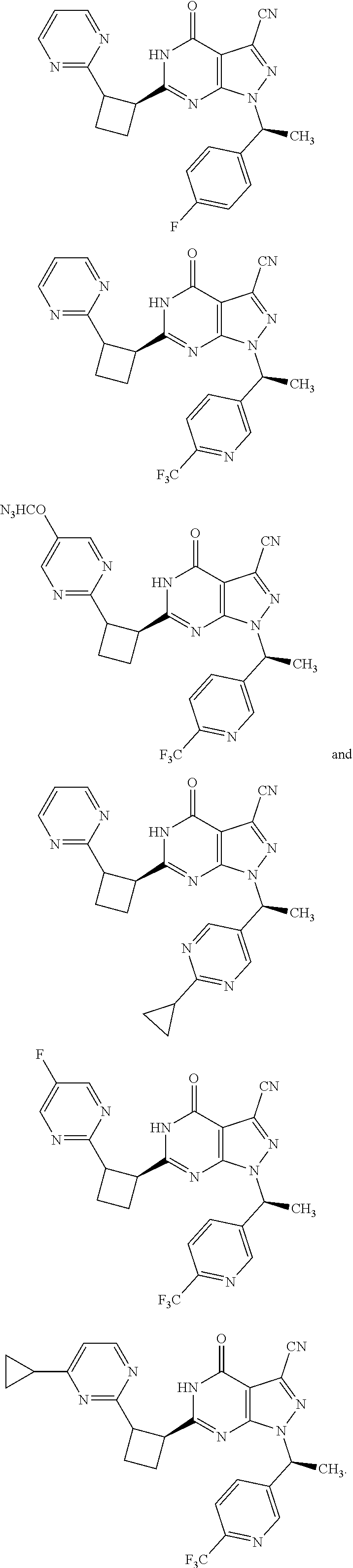

Embodiment 91. The method of any one of embodiments 1-12, wherein PDE9 inhibitor is represented by a compound of formula (AA-VI),

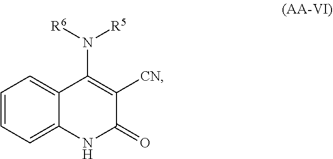

wherein
R⁵ is selected from the group consisting of:
   (1) hydrogen, and
   (2) —C₁₋₆alkyl;
R⁶ is —C₁₋₆alkyl, which is unsubstituted or substituted with oxetane;
or R⁵ and R⁶ are joined together with a —C₃₋₆alkyl through the nitrogen atom to which they are attached to form a azetidine, pyrrolidine, piperidine, or azepan ring, which is unsubstituted or substituted with pyrazole or one or two —C₁₋₆alkyl;
   or a pharmaceutically acceptable salt thereof.
      Embodiment 92. The method of any one of embodiments 1-12 wherein PDE9 inhibitor is selected from the group consisting of:

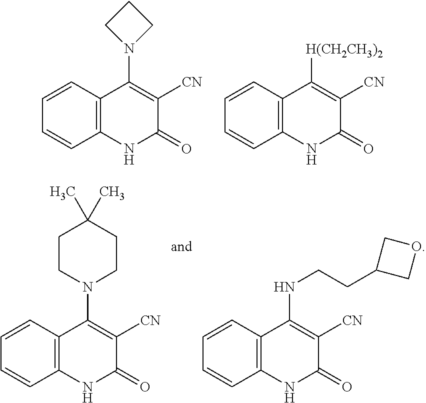

or a pharmaceutically acceptable salt thereof.
   Embodiment 93. The method of any one of embodiments 1-12, wherein PDE9 inhibitor is represented by a compound of formula (AA-VII),

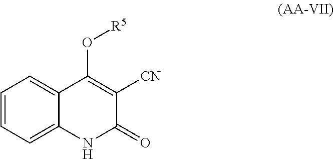

wherein:
R⁵ is selected from the group consisting of:
   (1) —C₁₋₆alkyl, which is unsubstituted or substituted with hydroxy, oxetane, or allyl,
   (2) -tetrahydropyranyl, and
   (3) —C₆₋₇cycloalkyl; or a pharmaceutically acceptable salt thereof.
      Embodiment 94. The method of any one of embodiments 1-12, wherein PDE9 inhibitor is represented by a compound of formula (AA-VIII),

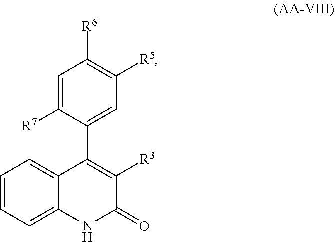

wherein
R³ is selected from the group consisting of:
   (1) —CN,
   (2) —CH₃, and
   (3) —CF₃:
R⁵, R⁶ and R⁷ are independently selected from the group consisting of:
   (1) hydrogen,
   (2) —C₁₋₆alkyl, and
   (3) fluoro,
   or R⁴ and R⁶ together with the carbons to which they are attached are joined together to form a fused phenyl ring; or a pharmaceutically acceptable salt thereof.
      Embodiment 95. The method of any one of embodiments 1-12, wherein PDE9 inhibitor is represented by a compound of formula (AA-IX),

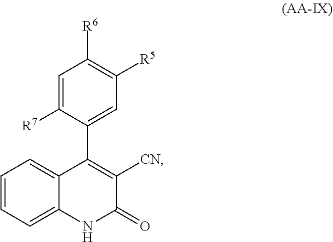

wherein
R⁶ is methyl or ethyl;
R⁵ is hydrogen; and
   R⁷ is hydrogen or fluoro
      Embodiment 96. The method of any one of embodiments 1-12, wherein PDE9 inhibitor is represented by a compound of formula (AA-X),

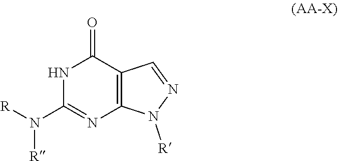

wherein
R′ is selected from the group consisting of isopropyl cyclopentyl, cyclohexyl, and isobutyl; and when R″═CH₃, R represents benzyl; and
when R″═H, R is selected from the group consisting of L-configured CHCH₃CONHR″′, D-configured CHCH₃CONHR″′, L-configured CH₂CONHR″′, D-configured CH₂CONHR″′, L-configured CH₂CH₂CONHR″′, D-configured CH₂CH₂CONHR″′, 3-methylpyridine, 1-phenylethyl, 1-(4-chlorophenyl)ethyl,

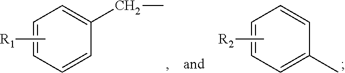

wherein R″′ is p-methoxyphenyl,
R₁ is selected from the group consisting of hydrogen, chlorine, methoxy, methyl, trifluoromethyl, dimethoxy, methylenedioxy, and dichlorine, and
R₂ is selected from the group consisting of hydrogen, methoxy, ethoxy, isopropoxy, methyl, dimethoxy, and 2-methyl-4-methoxy.
   Embodiment 97. The method of any one of embodiments 1-12, wherein PDE9 inhibitor is a compound having a structure of

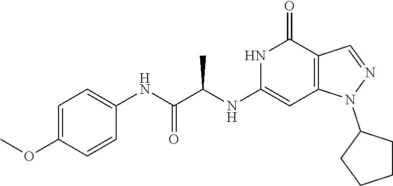 [SMILES: COc1ccc(NC(=O)[C@@H](C)Nc2cc3c(cnn3C3CCCC3)c(=O)[nH]2)cc1]

or a salt thereof.

1. A method of treating or preventing a disorder in a subject comprising: administering to the subject a therapeutically effective amount of a serotonergic psychedelic drug and a therapeutically effective amount of a PDE9 inhibitor, wherein the disorder comprises a neuropsychiatric disorder, cluster headache, or migraine headache,
wherein the PDE9 inhibitor mitigates at least one undesirable biological activity of the serotonergic psychedelic drug in the subject or reduces the abuse potential of the serotonergic psychedelic drug in the subject.

2. (canceled)

3. (canceled)

4. (canceled)

5. The method of claim 1, wherein the neuropsychiatric disorder is selected from the group consisting of a major depressive disorder (MIDD, including major depressive episode), a major depressive episode in bipolar disorder (bipolar depression), depressive episodes associated with bipolar I or II disorder (bipolar depression), a persistent depressive disorder (dysthymia), a disruptive mood dysregulation disorder, a premenstrual dysphoric disorder, a substance/medication-induced depressive disorder, a depressive disorder due to another medical condition, other specified depressive disorder, unspecified depressive disorder, an anxiety disorder, an obsessive-compulsive disorder, a posttraumatic stress disorder, an addictive disorder, treatment resistant depression (TRD), a generalized anxiety disorder (GAD), a post-traumatic stress disorder (PTSD), adjunctive treatment of major depression, eating disorders including anorexia and bulimia, depression associated with neurodegenerative disorders such as Parkinson's Disease, Alzheimer's Disease, dementias, and ALS, traumatic brain injury, suicidal thoughts and behaviors, chronic pain, a persistent depressive disorder, seasonal affective disorder (SAD), psychotic depression, peripartum (postpartum) depression, a premenstrual dysphoric disorder (PMDD), situational depression, and any combinations thereof.

6. The method of claim 1, wherein the serotonergic psychedelic drug and the PDE9 inhibitor are co-administered to the subject.

7. The method of claim 1, wherein the PDE9 inhibitor is administered to the subject before the serotonergic psychedelic drug is administered to the subject.

8. The method of claim 1, wherein the serotonergic psychedelic drug is administered to the subject before the PDE9 inhibitor is administered to the subject.

9. The method of claim 1, wherein the serotonergic psychedelic drug and the PDE9 inhibitor are co-formulated as a pharmaceutical composition.

10. The method of claim 1, wherein the serotonergic psychedelic drug and the PDE9 inhibitor are independently administered to the subject by a route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, intravenous and any combinations thereof.

11. The method of claim 1, wherein the serotonergic psychedelic drug is selected from the group consisting of Psilocin, Psilocybin, Bufotenin, Baeocystin, Aeruginascin, 5-MeO-DMT, N,N-Dimethyltryptamine (DMT), 5-Bromo-DMT, N-Methyl-N-ethyltryptamine (MET), N-Methyl-N-isopropyltryptamine (MiPT), N-Methyl-N-propyltryptamine (MPT), N,N-Diethyltryptamine (DET), N-Ethyl-N-isopropyltryptamine (EiPT), N-Methyl-N-butyltryptamine (MBT), N-Propyl-N-isopropyltryptamine (PiPT), N,N-Dipropyltryptamine (DPT), N,N-Diisopropyltryptamine (DiPT), N,N-Diallyltryptamine (DALT), N,N-Dibutyltryptamine (DBT), N-Ethyltryptamine (NET), N-Methyltryptamine (NMT), Trimethyltryptamine (TMT), α-Methyltryptamine, α-Ethyltryptamine, α,N-DMT, α,N,N-Trimethyltryptamine, Ethocybin, 4-HO-MET, 4-HO-DET, 4-HO-MPT, 4-HO-MiPT, 4-HO-MALT, 4-HO-DPT, 4-HO-DiPT, 4-HO-DALT, 4-HO-DBT, 4-HO-DSBT, 4-HO-αMT, 4-HO-MPMI, 4-HO-TMT, 4-HO-1,N,N-TMT, 4-HO-5-MeO-DMT, 4-AcO-DMT, 4-AcO-MET, 4-AcO-MALT, 4-AcO-DET, 4-AcO-EiPT, 4-AcO-DPT, 4-AcO-DiPT, 4-AcO-DALT, 4-MeO-DMT, 4-MeO-MiPT, 5-MeO—N MT, 5-MeO-MET, 5-MeO-MPT, 5-MeO-MiPT, 5-MeO-DET, 5-MeO-Ei PT, 5-MeO-EPT, 5-MeO-Pi PT, 5-MeO-DPT, 5-MeO-DiPT, 5-MeO-DALT, 5-MeO-αMT, 5-MeO-2,N,N-TMT, 5-MeO-7,N,N-TMT, 5-MeO-α,N-DMT, 4-F-5-MeO-DMT, 5-Me-MiPT, 5-HO-DiPT, 2-α-DMT, 4-Me-αMT, 4-Me-αET, 7-Me-αET, 4,5-DHP-AMT, 4,5-DH P-DMT, 4,5-MDO-DMT, 4,5-MDO-DiPT, 5,6-MDO-DiPT, 5,6-MDO-MiPT, 5-Fluoro-αMT, 6-Fluoro-αMT, 6-Fluoro-DMT, N,N-Tetramethyl enetryptamine (Pyr-T), 4-HO-pyr-T, 5-MeO-pyr-T, RU-28306 (4,α-Methylene-N,N-DMT), O-4310 (6-Fluoro-1-Isopropyl-4-HO-DMT), CP-132,484 (4,5-DHP-1-Methyltryptamine), Dimemebfe (5-MeO-BFE), 5-MeO-DiBF, Ibogaine, Voacangine, Lysergic acid diethylamide (LSD), Lysergic acid amide (LSA), Lysergic acid diamide, N1-Methyl-lysergic acid diethylamide, 1-Propionyl-lysergic acid diethylamide, 1-cyclopropanoyl-d-lysergic acid diethylamide, 1-valeryl-D-lysergic acid diethylamide, 6-Allyl-6-nor-lysergic acid diethylamide, 6-Butyl-6-nor-lysergic acid diethylamide, 6-Ethyl-6-nor-lysergic acid diethylamide, 1-Propionyl-6-Ethyl-6-nor-lysergic acid diethylamide, 6-Propyl-6-nor-lysergic acid diethylamide, 6-Cyclopropyl-6-nor-lysergic acid diethylamide, 6-nor-Lysergic acid diethylamide, Lysergic acid ethylamide, Lysergic acid α-hydroxyethylamide, Lysergic acid 2-butyl amide, Lysergic acid 3-pentyl amide, Lysergic acid methyl ester, Lysergic acid 2,4-dimethylazetidide, Lysergic acid piperidine, N,N-Dimethyl-lysergamide, Methylisopropyllysergamide, N,N-Diallyllysergamide, N-Pyrrolidyllysergamide, N-Morpholinyllysergamide, 1-methyl-lysergic acid butanolamide, Lysergic acid β-propanolamide, Lysergic acid 1-butanolamide, Mescaline, Lophophine, Isomescaline, Cyclopropylmescaline, Thioisomescaline (2-TIM, 3-TIM, and 4-TIM), 4-Desoxymescaline, Jimscaline, Escaline, Metaescaline, Thiometaescaline (3-TME, 4-TME, and 5-TME), Trisescaline, Thiotrisescaline (3-T-TRIS and 4-T-TRIS), Symbescaline, Asymbescaline, Thiosymbescaline (3-TSB and 4-TSB), Phenescaline, Allylescaline, Methallylescaline, Proscaline, Isoproscaline, Metaproscaline, Thioproscaline, Buscaline, Thiobuscaline, α-ethylmescaline, Ariadne, Macromerine, MEPEA, TOM (2-TOM and 5-TOM), Bis-TOM, TOMSO (2-methoxy-4-methyl-5-methylsulfinylamphetamine), TOET (2-TOET and 5-TOET), BOH, BOM (13-Methoxy-mescaline), beta-D, 4-D, DME, F-2, F-22, FLEA, MDPH, MDMP, Propynyl, 2C family (2,5-dimethoxy, 4-substituted phenethylamines), βk-2C-B, 2C—B, 2CB-2EtO, 2CB-5EtO, 2CB-diEtO, 2C—B-FLY, 2C—B-BUTTERFLY, 2C—C, 2C-D, 2CD-2EtO, 2CD-diEtO, 2CD-5EtO, 2C-E, 2C-EF, 2C—F, 2C-G (2C-G-1, 2C-G-2, 2C-G-3, 2C-G-4, 2C-G-5, 2C-G-6, and 2C-G-N), 2C—H, 2C—I, 2CI-2EtO, 2C-iP, 2C—N, 2C—O, 2C—O-4, 2C—P, 2C-SE, 2C-T, 2CT-5EtO, 2C-T-2, 2CT-2-2EtO, 2CT-2-5EtO, 2CT-2-diEtO, 2C-T-4 (2C-T-4 and Ψ-2C-T-4), 2CT-4-2EtO, 2C-T-7, 2CT-7-2EtO, 2C-T-8, 2C-T-9, 2C-T-13, 2C-T-15, 2C-T-16, 2C-T-17, 2C-T-19, 2C-T-21, 2C-TFM, 2C—YN, BOB (I3-Methoxy-2C-B), BOD (I3-Methoxy-2C-D), BOHD (I3-Hydroxy-2C-D), HOT-2, HOT-7, HOT-17, Indane derivatives comprising 2CB-Ind, Benzocyclobutene derivatives comprising 2C—BCB (TCB-2), NBOMe derivatives comprising NBOMe-mescaline, 2C—H—NBOMe, 2C—C—NBOMe, 2CBCB—NBOMe, 2CBFly-NBOMe, 2C—B—NBOMe, 2C—I—NBOMe, 2C-TFM-NBOMe, 2C-D-NBOMe, 2C-G-NBOMe, 2C-E-NBOMe, 2C—P—NBOMe, 2C-iP-NBOMe, 2C—CN—NBOMe, 2C—N—NBOMe, 2C-T-NBOMe, 2C-T-4-NBOMe, 2C-T-7-NBOMe, and DMBMPP, NBOH derivatives comprising 2C—C—NBOH, 2C—B—NBOH, 2C—I—NBOH, and 2C—CN—NBOH, NBMD derivatives comprising 2C—I-NBMD, NBF derivatives comprising 2C—C—NBF, 2C—B—NBF, and 2C—I—NBF, 3C family (3,5-dimethoxy, 4-substituted amphetamines) comprising 3C-E, 3C—P, 3C-DFE, and 3C—BZ, DOx family (2,5-dimethoxy, 4-substituted amphetamines) comprising DOAM, DOB, Meta-DOB, Methyl-DOB, DOBU, DOC, DOEF, DOET, DOI, DOM, Ψ-DOM, DON, DOPR, SOiPR, DOT, Meta-DOT, Ortho-DOT, and DOTFM, DMCPA, DMMDA, DMMDA-2, 2,5-dimethoxy-3,4-dimethylamphetamine, 4-methyl-2,5-dimethoxymethamphetamine, 2,N-dimethyl-4,5-methylenedioxyamphetamine, Dimethoxyamphetamine (2,4-DMA, 2,5-DMA, and 3,4-DMA), Trimethoxyamphetamine (TMA-2, TMA-6), Tetramethoxyamphetamine, Br-DragonFLY, TFMFly, 2-Bromo-4,5-methylenedioxyamphetamine, 4-Bromo-3,5-dimethoxyamphetamine, EEE, EEM, EME, EMM, EDMA, EIDA, Ethyl-J, Methyl-J, Ethyl-K, Mehtyl-K, IDNNA, Iris, MDAI, MDMAI, MDAT, MDMAT, MDAL, MDBU, MDBZ, MDDM, MDIP, MDMEOET, MDMEO, MDOH, MDHOET, MDPL, MDCPK, MDPR, MEDA, MEM, Mehtyl-DMA, MMDA, MMDA-2, 5-Mehtyl-MDA, MEE, MME, MPM, DiFMDA, 5-APB, 6-APB, 5-APDB, 6-APDB, 5-MAPB, 5-MAPDB, 6-MAPDB, 6-MAPB, 6-EAPB, 5-EAPB, Para-Methoxyamphetamine, Paramethoxymethamphetamine, 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Benzoxazine, Efavirenz, Substituted methylenedioxy-phenethylamines (MDxx) comprising 3,4-methylenedioxymethamphetamine (MDMA), MDA, 2,3-MDA, 5-Methyl-MDA, MMDA, MDEA, MBDB, MDAL, MDBU, MDBZ, MDDM, MDIP, MDMEOET, MDMEO, MDOH, MDHOET, MDPL, MDCPM, MDPR, BDB, MMDA-2, DiFMDA, EIDA, Ethyl-K, Lophophine, Substituted amphetamines, EDMA, Para-Methoxyamphetamine (PMA), Paramethoxymethamphetamine (PMMA), 4-Ethylamphetamine, 3-Methoxy-4-methylamphetamine, 4-Methylmethamphetamine, 4-Methylthioamphetamine, 4-Fluoroamphetamine, Norfenfluramine, Para-Iodoamphetamine, Para-Chloroamphetamine, Substituted cathinones, Methylone, Ethylone, Eutylone, Butylone, Pentylone, 4-Ethyl methcathinone, 3-Methylmetheathinone, Substituted benzofurans, 5-AP B, 6-AP B, 5-APDB, 6-APDB, 5-MAPB, 5-MAPDB, 6-MAPDB, 6-MAPB, 5-EAPB, 6-EAPB, 5-MBPB, MDAT, MDMAT, 6-CAT, Tetralinylaminopropane, Trifluoromethylaminoindane, Ethyltrifluoromethylaminoindane, 5-Iodo-2-aminoindane, MMAI, MDAI, MDMAI, Indanylaminopropane, Naphthylaminopropane, 4-chlorophenylisobutylamine,
4-Methylphenylisobutylamine, Ariadne, α-methyltryptamine, 5-MeO-αMT, α-ethyltryptamine, 4-Me-αET, 7-Me-αET, 5-MeO-αET, 5-MeO-MiPT, Δ⁹-THC, CBD, CBN, THCV, (C₆)—CP 47,497, (C₉)—CP 47,497, 1-Butyl-3-(2-methoxybenzoyl)indole, 1-Butyl-3-(4-methoxybenzoyl)indole, 1-Pentyl-3-(2-methoxybenzoyl)indole, 2-Isopropyl-5-methyl-1-(2,6-di hydroxy-4-nonylphenyl)cyclohex-1-ene, 4-HTMPIPO, 4-Nonylphenylboronic acid, 5Br—U R-144, 5Cl-APINACA, 5Cl—U R-144, 5F-3-pyridinoylindole, 5F-AB-FUPPYCA, 5F-ADB-PINACA, 5F-ADBICA, 5F-ADB, 5F-AMB, 5F-APINACA, 5F-CUMYL-PINACA, 5F-EMB-PINACA, 5F-NNE1, 5F—PB-22, 5F—PCN, 5F—PY-PICA, 5F—PY—P INACA, 5F-SDB-006, HHC, A-796,260, A-834,735, A-836,339, A-955,840, A-40174, A-41988, A-42574, AB-001, AB-CH FU PYCA, AB-CHMFUPPYCA, AB-CHMINACA, AB-FUBICA, AB-FUBINACA 2-fluorobenzyl isomer, AB-FUBINACA, AB-PICA, AB-PINACA, Abnormal cannabidiol, ADAMANTYL-THPINACA, ADB-CHMINACA, ADB-FUBICA, ADB-FUBINACA, ADB-PINACA, ADBICA, ADS B—FU B-187, Ajulemic acid, AM-087, AM-411, AM-630, AM-679, AM-694, AM-855, AM-883, AM-905, AM-906, AM-919, AM-926, AM-938, AM-1220, AM-1221, AM-1235, AM-1241, AM-1248, AM-1346, AM-1387, AM-1714, AM-2201, AM-2232, AM-2233, AM-2389, AM-4030, AM-4113, AM-6527, AM-6545, AM-251, AM-281, AM-404, AMB-CHMINACA, AMB-FUBINACA, AMG-1, AMG-3, AMG-36, AMG-41, APICA, APINACA, APP-FUBINACA, Arachidonoyl SEROTONIN, ACEA, ACPA, Arvanil, AZ-11713908, BAY 38-7271, BAY 59-3074, BIM-018, Biochanin A, BML-190, Nabidrox (Canbisol), Cannabicyclohexanol, Cannabipiperidiethanone, CAY-10401, CAY-10429, CAY-10508, CB-13, CB-25, CB-52, CB-86, CB-86, CBS-0550, CP 47,497, CP 55,244, CP 55,940, CUMYL-5F-PICA, CUMYL-BICA, CUMYL-PICA, CUMYL-PINACA, CUMYL-THPINACA, Dexanabinol, Dimethylheptylpyran, Drinabant, Dronabinol, EAM-2201, EMB-FUBINACA, FAB-144, FDU-NNE1, FDU-PB-22, FUB-144, FUB-APINACA, FUB-JWH-018, FUB—PB-22, FUBIMINA, Genistein, GW-405,833, GW-842,166X, Hemopressin, HU-210, HU-243, HU-308, HU-320, HU-331, HU-336, HU-345, HU-910, Ibipinabant, IDFP, JNJ 1661010, JNJ 1661010, JTE-907, JTE 7-31, JWH-007, JWH-015, JWH-018, JWH-019, JWH-030, JWH-051, JWH-073, JWH-081, JWH-098, JWH-116, JWH-122, JWH-133, JWH-139, JWH-147, JWH-149, JWH-161, JWH-164, JWH-167, JWH-175, JWH-176, JWH-182, JWH-184, JWH-185, JWH-192, JWH-193, JWH-194, JWH-195, JWH-196, JWH-197, JWH-198, JWH-199, JWH-200, JWH-203, JWH-210, JWH-229, JWH-249, JWH-250, JWH-251, JWH-302, JWH-307, JWH-359, JWH-369, JWH-370, JWH-398, JWH-424, JZL184, JZL195, Kaempferol, KM-233, L-759,633, L-759,656, LASSBio-881, LBP-1, Leelamine, Levonantradol, LH-21, LY-320,135, LY-2183240, NAM-2201, MDM-2201, MDA-7, MDA-19, MDA-77, MDMB-CHMICA, MDMB-CHMINACA, MDMB-FUBINACA, Menabitan, MEPIRAPIM, Methanandamide, MJ-15, MK-9470, MMB-2201, MN-18, MN-25, Nabazenil, Nabilone, Nabitan, Naboctate, NESS-0327, NESS-040C₅, NIDA-41020, NM-2201, NMP-7, NNE1, Nonabine O-224, O-581, O-585, O-606, O-689, O-774, O-806, O-823, O-889, O-1057, O-1125, O-1184, O-1191, O-1238, O-1248, O-1269, O-1270, O-1376, O-1399, O-1422, O-1601, O-1602, O-1624, O-1656, O-1657, O-1660, O-1812, O-1860, O-1861, O-1871, O-1918, O-2048, O-2050, O-2093, O-2113, O-2220, O-2365, O-2372, O-2373, O-2383, O-2426, O-2484, O-2545, O-2654, O-2694, O-2715, O-2716, O-3223, O-3226, Oleoylethanolamide, Olvanil, Org 27569, Org 27759, Org 2831, Org 28611, Org 29647, Otenabant, Palmitoylethanolamide, Parahexyl, PF-03550096, PF-04457845, PF-622, PF-750, PF-3845, PF-514273, PHOP, PipISB, Pirnabine, Pravadoline, Pregnenolone, PSB—SB-487, PSB—SB-1202, PTI-1, PTI-2, PX-1, PX-2, PX-3, QUCHIC, QUPIC, RCS-4, RCS-8, Rimonabant, Rosonabant, RTI-371, S-444,823, SDB-006, SER-601, SPA-229, SR-144,528, STS-135, Surinabant, Taranabant, Tedalinab, THC-O-acetate, THC-O-phosphate, THJ-018, THJ-2201, Tinabinol, TM-38837, UR-144, URB-447, URB-447, URB-597, URB-602, URB-754, VCHSR, VDM-11, VSN-16, WIN 54,461, WIN 55,212-2, WIN 56,098, XLR-11, Yangonin, Harmaline, harmala alkaloids, other beta-carbolines, active constituents of ayahuasca, Salvinorin A, Salvinorin B methoxymethyl ether, Salvinorin B ethoxymethyl ether, Piperazines, pFPP, TFMPP, Myristicin, Elemicin, Cryogenine (Vertine), Atropine, Scopolamine, Hyoscyamine, Ibotenic acid, Muscimol, TiHKAL, 2-Me-DET, 4-HO-DBT, 4-HO-DET, 4-HO-DiPT, 4-HO-EPT, 4-HO-McPT, 4-HO-MET, 4-HO-MiPT, 4-HO-MPMI, 4-HO-MPT, 4-HO-αMT, 4-Me-αMT, 4-MeO-MiPT, 4-PrO-DMT, 4,5-MDO-DiPT, 4,5-MDO-DMT, 5-Ethoxy-αMT, 5-Fluoro-AET, 5-Fluoro-DET, 5-Fluoro-DMT, 5-Fluoro-EPT, 5-Fluoro-MET, 5-MeO-AET, 5-MeO-αMT, 5-MeO-DALT, 5-MeO-DET, 5-MeO-DPT, 5-MeO-EiPT, 5-MeO-MALT, 5-MeO-MiPT, 5-MeO-MPMI, 5-MeO-pyr-T, 5-MeS-DMT, 5-MeO-2-TMT, 5-TFM-DMT, 5-TFMO-DMT, 5,6-MDO-DiPT, 5,6-MDO-DMT, 5,6-MDO-MiPT, 5,6-MeO-MiPT, 5,N,N-TMT, 5F-MPMI, 6-Fluoro-DET, 6-Fluoro-DMT, 6-MeO-THH, 7-Chloro-AMT, 7-F-5-MeO-MET, 4-Acetoxy-DET, 4-Acetoxy-DiPT, 4-Acetoxy-MET, 4-Acetoxy-MiPT, O-Acetylbufotenine, O-Acetylpsilocin, Aeruginascin, Alpha,N-DMT, Alpha,N,O-TMS, Baeocystin, 5-Bromo-DMT, Bufotenin, 5-Chloro-αMT, DALT, Dibutyltryptamine, Diethyltryptamine, Diisopropyltryptamine, 5,N-Dimethyl-N-isopropyltryptamine, Dimethyltryptamine, N,N-Dimethyltryptamine, Dipropyltryptamine, 2,α-Dimethyltryptamine, Ethocybin, Ethylisopropyltryptamine, A-Ethyltryptamine, 5-Fluoro-AMT, 4-Fluoro-5-methoxy-DMT, FT-104, 5-MeO-DMT, 5-Methoxy-N,N-diisopropyltryptamine, 4-Methyl-α-ethyltryptamine, N-Methyl-N-ethyltryptamine, Methylbutyltryptamine, Methylisopropyltryptamine, Alpha-Methylserotonin, Alpha-Methyltryptamine, N-Methyltryptamine, MPMI, Norbaeocystin, Propylisopropyltryptamine, 2,N,N-TMT, A,N,N-Trimethyltryptamine, Ketamine, Esketamine, Arketamine, Mitragynine, Yohimbine, and any combinations thereof.

12. The method of claim 1, wherein the serotonergic psychedelic drug is a tryptamine or an ergoline, or a derivative thereof, or a salt, solvate, enantiomer, or diastereomer thereof.

13. The method of claim 12, wherein the serotonergic psychedelic drug comprises a tryptamine, or a derivative thereof, or a salt, solvate, enantiomer, or diastereomer thereof.

14. The method of claim 12, wherein the serotonergic psychedelic drug comprises an ergoline, or a derivative thereof, or a salt, solvate, enantiomer, or diastereomer thereof.

15. The method of claim 11, wherein the serotonergic psychedelic drug is psilocybin, or a derivative thereof, or a salt, solvate, enantiomer, or diastereomer thereof.

16. The method of claim 1, wherein the PDE9 inhibitor is selected from the group consisting of aminophylline, pentoxifylline, theobromine, paraxanthine, PF-04447913, PF-04447943, PF-04447953, BAY 73-6691, E 2027, CRD 773, BI 409306, CRD 740, TT 00920, BAY 7081, FRM 16606, HUC1-288, WYQ-C₃₆D, and any combinations thereof.

17. The method of claim 1, wherein the PDE9 inhibitor is PF-04447943, or a derivative thereof, or a salt, solvate, enantiomer or diastereomer thereof.

18. The method of claim 1, wherein the subject is administered with a therapeutically effective amount of psilocybin and PF-04447943.

19. The method of claim 1, wherein the subject is administered with a therapeutically effective amount of psilocybin, or a derivative thereof, or a salt, solvate, enantiomer or diastereomer thereof and PF-04447943, or a derivative thereof, or a salt, solvate, enantiomer or diastereomer thereof.

20. The method of claim 1, wherein the PDE9 inhibitor is selected from the group consisting of 1-{[2-(3-isopropyl-7-oxo-6,7-dihydro-1H-pyrazolo[4,3-d]pyrimidin-5-ylmethyl)-phenoxy]-acetyl}-pyrrolidine-2-carbo-xylic acid; 1-{[2-(1-cyclopentyl-4-oxo-4,5-dihydro-1H-pyrazolo[3,4-d]pyrim-idin-6-ylmethyl)-phenoxy]-acetyl}-pynOlidine-2(S)-carboxylic acid 3-isopropyl-5-[2-(2-oxo-2-piperazin-1-yl-ethoxy)-benzyl]-1,6-dihydro-pyra-zolo[4,3-d]pyrimidin-7-one; 1-cyclopentyl-6-[2-(2-oxo-2-piperazin-1-yl-eth-oxy)-benzyl]-1,5-dihydro-pyrazolo[3,4-d]pyrimidin-4-one 3-isopropyl-5-[2-(2-morpholin-4-yl-2-oxo-ethoxy)-benzyl]-1,6-dihydro-pyra-zolo[4,3-d]pyrimidin-7-one; 3-isopropyl-5-[2-(2-oxo-2-pyrrolidin-1-yl-etho-xy)-benzyl]-1,6-dihydro-pyrazolo[4,3-d]pyrimidin-7-one; 5-{2-[2-(4-ethyl-piperazin-1-yl)-2-oxo-ethoxy]-benzyl}-3-isopropyl-1,6-di-hydro-pyrazolo[4,3-d]pyrimidin-7-one; N,N-diethyl-2-[2-(3-isopropyl-7-oxo-6,7-dihydro-1H-pyrazolo[4,3-d]pyrimidin-5-ylmethyl)-phenoxy]-acetamide; 1-{[2-(3-isopropyl-7-oxo-6,7-dihydro-1H-pyrazolo[4,3-d]pyrimidin-5-ylmeth-yl)-phenoxy]-acetyl}-pyrrolidine-2-carboxy lie acid methyl ester; 4-{[2-(3-isopropyl-7-oxo-6,7-dihydro-1H-pyrazolo[4,3-d]pyrimidin-5-ylmeth-yl)-phenoxy]-acetyl}-piperazine-1-carboxylic acid tert-butyl ester; N-(2-dimethylamino-ethyl)-2-[2-(3-isopropyl-7-oxo-6,7-dihydro-1H-pyrazolo-[4, 3-d]pyrimidin-5-ylmethyl)-phenoxy]-acetamide; 1-{[2-(1-cyclopentyl-4-ox-o-4,5-dihydro-1H-pyrazolo[3,4-d]pyrimidin-6-ylmethyl)-phenoxy]-acetyl}-pyr-rolidine-2-carboxylic acid methyl ester; 4-{[2-(1-cyclopentyl-4-oxo-4,5-di-hydro-1H-pyrazolo[3,4-d]pyrimidin-6-ylmethyl)-phenoxy]-acetyl}-piperazine-1-carboxylic acid tert-butyl ester; 1-cyclopentyl-6-[2-(2-oxo-2-pyrrolidin-1-yl-ethoxy)-benzyl]-1,5-dihydro-pyrazolo[3,4-d]pyrimidin-4-one; 1-cyclopentyl-6-[2-(2-morpholin-4-yl-2-oxo-ethoxy)-benzyl]-1,5-dihydro-py-razolo[3,4-d]pyrimidin-4-one; 2-[2-(1-cyclopentyl-4-oxo-4,5-dihydro-1H-pyr-azolo[3,4-d]pyrimidin-6-ylmethyl)-phenoxy]-N-(2-dimethylamino-ethyl)-aceta-mide; 1-cyclopentyl-6-{2-[2-(4-ethyl-piperazin-1-yl)-2-oxo-ethoxy]-benzyl}-1,5-dihydro-pyrazolo[3,4-d]pyrimidin-4-one; 2-[2-(1-cyclopentyl-4-oxo-4,5-dihydro-1H-pyrazolo[3,4-d]pyrimidin-6-ylmethyl)-phenoxy]-N,N-diethyl-acetamide; [2-(3-isopropyl-7-oxo-6, 7-dihydro-1H-pyrazolo[4,3-d]pyrimidin-5-ylm-ethyl)-phenoxy]-acetic acid; [2-(1-cyclopentyl-4-oxo-4,5-dihydro-1H-pyrazo-lo[3,4-d]pyrimidin-6-ylmethyl)-phenoxy]-acetic acid; 3-isopropyl-5-[2-(5-chloro-2-morpholin-4-yl-ethoxy)-benzyl]-1,6-dihydro-p-yrazolo[4,3-d]pyrimidin-7-one; 3-isopropyl-5-[2-(2-pyrrolidin-1-yl-ethoxy)-benzyl]-1,6-dihydro-pyrazolo[4,3-d]pyrimidin-7-one; 3-isopropyl-5-[2-(2-morpholin-4-yl-ethoxy)-cy cl ohexylmethyl]-1, 6-dihydro-pyrazolo[4,3-d]pyrimidin-7-one; 5-[5-fluoro-2-(2-morpholin-4-yl-ethoxy)-benzyl]-3-isopropyl-1,6-dihydro-pyrazolo[4,3-d]pyrimidin-7-one; 3-cyclopentyl-5-[5-fluoro-2-(2-morpholin-4-yl-ethoxy)-benzyl]-1, 6-dihydro-1-pyrazolo[4,3-d]pyrimidin-7-one; 3-isopropyl-5-[2-(2-morpholin-4-yl-ethoxy-benzyl]-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 2-[2-(2-morpholin-4-yl-ethoxy)-benzyl]-9-(tetrahydro-furan-3-yl)-1,9-dihydro-purin-6-one; 5-[2-(2-diethylamino-ethoxy)-benzyl]-3-isop-ropyl-1,6-dihydro-pyrazolo[4,3-d]pyrimidin-7-one; 3-cyclopentyl-5-[2-(2-mo-rpholin-4-yl-ethoxy)-benzyl]-1,6-dihydro-pyrazolo[4,3-d]pyrimidin-7-one; 3-cyclobutyl-5-[2-(2-morpholin-4-yl-ethoxy)-benzyl]-1,6-dihydro-pyrazolo[4,3-d]pyrimidin-7-one; 9-(1(R),2-dimethyl propyl)-[2-(2-morpholin-4-yl-eth-oxy)-benzyl]-1,9-dihydro-purin-6-one; 9-(2-methyl-butyl)-2-[2-(2-morpholin-4-yl-ethoxy)-benzyl]-1,9-dihydro-purin-6-one; 9-cyclopentyl-2-[2-(2-morph-olin-4-yl-ethoxy)-benzyl]-1,9-dihydro-purin-6-one; 5-[2-(2-morpholin-4-yl ethoxy)-benzyl]-3-pyridin-3-yl-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 9-isopropyl-2-[2-(2-morpholin-4-yl-ethoxy)-benzyl]-1,9-dihy-dro-purin-6-one; 2-[2-(2-morpholin-4-yl-ethoxy)-benzyl]-9-(tetrahy dro-fura-n-2-ylmethyl)-1,9-dihydro-purin-6-one; 9-(1-isopropyl-2-methyl-propyl)-2-[-2-(2-morpholin-4-yl-ethoxy)-benzyl]-1,9-dihydro-purin-6-one; 9-(1-ethyl-propyl)-2-[2-(2-morphobn-4-yl-ethoxy)-benzyl]-1, 9-dihydro-pur-in-6-one; 9-cyclopentyl-8-methyl-2-[2-(2-morphobn-4-yl-ethoxy)-benzyl]-1,-9-dihydro-purin-6-one; 3-cyclopentyl-5-[2-(2-morphobn-4-yl-ethoxy)-benzyl-]-3,6-dihydro-[1,2,3]triazolo[4,5-d]pyrimidin-7-one; 1-cyclopentyl-6-[2-(2-morpholin-4-yl-ethoxy)-benzyl]-1,5-dihydro-pyrazolo-[3,4-d]pyrimidin-4-one; 9-cyclopentyl-2-[2-(3-morpholin-4-yl-propoxy)-benz-yl]-1,9-dihydro-purin-6-one; N-[(1R,2S)₂-(3-isopropyl-7-oxo-6,7-dihydro-1H-pyrazolo[4,3-d]pyrimidin-5-ylmethyl)-cyclohex-1-yl]-2-pyrrobdin-1-yl-ace-tamide; N-[(1R,2S)2-(3-isopropyl-7-oxo-6,7-dihydro-1H-pyrazolo[4,3-d]pyrim-idin-5-ylmethyl)-cyclohex-1-yl]-2-morphobn-4-yl-acetamide; 2-diethylamino-N-[(1R,2S)2-(3-isopropyl-7-oxo-6,7-dihydro-1H-pyrazolo[4,3-d]pyrimidin-5-ylmethyl)-cyclohex-1-yl]-acetamide; 1-{[(1R,2S)2-(3-isoprop-yl-7-oxo-6,7-dihydro-1H-pyrazolo[4,3-d]pyrimidin-5-ylmethyl)-cyclohex-1-yl-carbamoyl]-methyl}-pyrrobdine-2(S)-carboxy lie acid methyl ester; 2-cyclobutylamino-N-[(1R,2S)2-(3-isopropyl-7-oxo-6,7-dihydro-1H-pyrazolo[-4,3-d]pyrimidin-5-ylmethyl)-cyclohex-1-yl]-acetamide; or 2-cyclopropylamino-N-[(1R,2S)2-(3-isopropyl-7-oxo-6,7-dihydro-1H-pyrazolo-[4,3-d]pyrimidin-5-ylmethyl)-cyclohex-1-yl]-acetamide; IMR-687, (a potent inhibitor of PDE9A), BAY73-6691, and PF-04447943, PF-4181366, and stereoisomers, pharmaceutically acceptable salts, solvates and prodrugs thereof, and any combinations thereof.

21. The method of claim 1, wherein the PDE9 inhibitor is selected from the group consisting of:
a salt of (S)-7-(2-methoxy-3,5-dimethylpyridin-4-yl)-1-(tetrahydrofuran-3-yl)-1H-pyrazolo[4,3-c]quinolin-4(5H)-one and an acid selected from the group consisting of hydrochloric acid, hydrobromic acid, sulfuric acid, nitric acid, phosphoric acid, malonic acid, maleic acid, tartaric acid, methanesulfonic acid, benzenesulfonic acid and toluenesulfonic acid;
(S)-7-(2-methoxy-3,5-dimethylpyridin-4-yl)-1-(tetrahydrofuran-3-yl)-1H-pyrazolo[4,3-c]quinolin-4(5H)-one monomaleate salt;
(S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt;
a crystal of the salt;
a crystal of (S)-7-(2-methoxy-3,5-dimethylpyridin-4-yl)-1-(tetrahydrofuran-3-yl)-1H-pyrazolo[4,3-c]quinolin-4(5H)-one monomaleate salt, having a diffraction peak at a diffraction angle (2θ±0.2°) of 10.1° in powder X-ray diffraction; and
a crystal of (S)-7-(2-methoxy-3,5-dimethylpyridin-4-yl)-1-(tetrahydrofuran-3-yl)-1H-pyrazolo[4,3-c]quinolin-4(5H)-one monobenzenesulfonate salt, having a diffraction peak at a diffraction angle (2θ±0.2°) of 9.9° in powder X-ray diffraction.

22-34. (canceled)

35. The method of claim 1, wherein the serotonergic psychedelic drug is not masculine.

36-62. (canceled)

63. The method of claim 11, wherein the serotonergic psychedelic drug is 5-MeO-DMT.
